SEQUENCE LISTING (241 sequences)

SEQ ID NO: 1 - protein, 82 aa, synthetic construct
VAMRSCPEEQYWDPLLGTCMSCKTICNHQSQRTCAAFCRSLSCRKEQGKFYDHLLRDCISCASICGQHPKQCAYFCENKLRS

SEQ ID NO: 2 - protein, 82 aa, synthetic construct
VAMRSCPEEQYWDPLLGTCMSCKTICNHQSQRTCAAFCRSLSCRKEQGKFYDHPLRDCISCASICGQHPKQCAYFCENKLRS

SEQ ID NO: 3 - protein, 82 aa, synthetic construct
VAMRSCPEEQYWDPLLGTCMSCKTICNHQSQRTCAAFCRSLSCRKEQGKFYDHLLRECISCASICGQHPTQCAYFCENKLRS

SEQ ID NO: 4 - protein, 82 aa, synthetic construct
VAMRSCPEEQYWDPLLGTCMSCKTICNHQSQRTCAAFCRSLSCRKEQGKFYDHLLRDCLSCASICGQHPTQCAYFCENKLRS

SEQ ID NO: 5 - protein, 82 aa, synthetic construct
VAMRSCPEEQYWDPLLGTCMSCKTICNHQSQGTCAAFCRSLSCRKEEGKFYDHPLRDCISCASICGQHPKQCAYFCENKLRS

SEQ ID NO: 6 - protein, 82 aa, synthetic construct
VAMRSCPEEQYWDPLLGTCMSCKTICNHQSQGTCAAFCRSLSCRKEQGKFYDHLLRDYISCASICGQHPKQCAYFCENKLRS

SEQ ID NO: 7 - protein, 82 aa, synthetic construct
VAMRSCPEEQYWDPLLGTCMSCKTICNHQSQRTCAAFCRSLSCRKEQGKFYDHLLRDCISCASICGQHPKQCDYFCENKLRS

SEQ ID NO: 8 - protein, 82 aa, synthetic construct
VAMRSCPEEQYWDPLLGTCMSCKTICNHQSQRTCAAFCRSLSCRKEQGKFYDHLLRDYISCASICGQHPKQCAYFCENKLRS

SEQ ID NO: 9 - protein, 82 aa, synthetic construct
VAMRSCPEEQYRDPLLGTCMSCKTICNHQSQRTCAAFCRSLSCRKEQGKFYDHPLRDCISCASICGQHPKQCAYYCENKLRS

SEQ ID NO: 10 - protein, 82 aa, synthetic construct
VAMRSCPEEQYRDPLLGTCMSCKTICNHQSRRPCAAFCRSLSCRKEQGKFYDHLLRDCISCASICGQHPTQCAYFCENKLRS

SEQ ID NO: 11 - protein, 82 aa, synthetic construct
VAMRSCPEEQYWDPLLGTCMSCKTICNHQSQRTCAAFCRSLSCRKEQGKFYDHPLRDCLSCASICGQHPKQCAYFCENKLRS

SEQ ID NO: 12 - protein, 82 aa, synthetic construct
VAMRSCPEEQYWDPLLGTCMSCKTICNHQSQRTCAAFCRSLSCRKEQSKFYDHLLRDCISCASICGQHSKQCAYFCENKLRS

SEQ ID NO: 13 - protein, 43 aa, synthetic construct
SLSCRKEQGKFYDHLLRDCISCASICGQHPKQCAYFCENKLRS

SEQ ID NO: 14 - protein, 43 aa, synthetic construct
SLSCRKEQGKFYDHLLRVCISCASICGQHPKQCAYFCENKLRS

SEQ ID NO: 15 - protein, 43 aa, synthetic construct
SLSCRKVQGKFYDHLLRDCISCASICGQHPKQCAYFCENKLRS

SEQ ID NO: 16 - protein, 43 aa, synthetic construct
SLSCRKEQGKFYDHLLLDCISCASICGQHPKQCAYFCENKLRS

SEQ ID NO: 17 - protein, 43 aa, synthetic construct
SLSCRKEQGEFYDHLLLDCISCASICGQHPKQCAYYCENKLRS

SEQ ID NO: 18 - protein, 43 aa, synthetic construct
SLSCRKEQGKFFDHLLRDCISCASICGQHPKECAYFCENKLRS

SEQ ID NO: 19 - protein, 43 aa, synthetic construct
SLSCRKEQGKFFDHLLRDCISCASICGQHPKQCAYFCENKLRS

SEQ ID NO: 20 - protein, 43 aa, synthetic construct
SLSCRKEQGKFYDHLLGDCISCASICGQHPKQCAYFCENKLRS

SEQ ID NO: 21 - protein, 82 aa, synthetic construct
VAMRSCPEEQYWDPLLGTCMSCKTICNHQSQRTCAAFCRSLSCRKEQGKFYDHLSRDCISCASICGQHPKQCAYSCENKLRS

SEQ ID NO: 22 - protein, 82 aa, synthetic construct
VAMRSCPEEQYWDPLLGTCMSCKTICNHQSQRTCAAFCRSLSCRKEQGKFYDHHLRDCISCASICGQHPKQCAYFCENKLRS

SEQ ID NO: 23 - protein, 43 aa, synthetic construct
SLSCRKEQGKFYDHLLRDCISCASICGQHPKQCDYFCENKLRS

SEQ ID NO: 24 - protein, 43 aa, synthetic construct
SLSCRKEQGEFYDHLLQDCISCASICGQHPKQCAYFCENKLRS

SEQ ID NO: 25 - protein, 43 aa, synthetic construct
SLSCRKEQGEFYDHLLRDCISCASICGQHPKQCDYFCENKLRS

SEQ ID NO: 26 - protein, 43 aa, synthetic construct
SLSCRKEQGEYYDHLLRDCISCASICGQHPKQCADFCENKLRS

SEQ ID NO: 27 - protein, 43 aa, synthetic construct
SLSCRKEEGKFYDHLLQDCISCASICGQHPKQCAYFCENKLRS

SEQ ID NO: 28 - protein, 43 aa, synthetic construct
SLSCRKERGKFYDHLLGDCISCASVCGQHPKQCAYFCENKLRS

SEQ ID NO: 29 - protein, 43 aa, synthetic construct
SLSCRKEQGEFYDHLLRDCISCASICGQHPKQCDDFCENKLRS

SEQ ID NO: 30 - protein, 43 aa, synthetic construct
SLSCRKEQGKFYDHLLQDCISCASICGQHPKQCAYFCENKLRS

SEQ ID NO: 31 - protein, 43 aa, synthetic construct
SLSCRKEQGKFYDHLLQDCINCASICGQHPKQCDYFCENKLRS

SEQ ID NO: 32 - protein, 43 aa, synthetic construct
SLSCRKEQGEFYDHLLRDCISCASICGQHPKQCAYFCENKLRS

SEQ ID NO: 33 - protein, 43 aa, synthetic construct
SLSCRKEQGKFYDHLLQDCISCASICGQHPKQCAYVCENKLRS

SEQ ID NO: 34 - protein, 43 aa, synthetic construct
SLSCRKEQGEFYDHLLRDCISCASICGRHPKQCDYFCENKLRS

SEQ ID NO: 35 - protein, 43 aa, synthetic construct
SLSCRKEQGKFYDHLLRDCMSCASICGQHPKQCDYFCENKLRS

SEQ ID NO: 36 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatct

SEQ ID NO: 37 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcctttgcgagactgtataagctgtgcgtctatttgtggacaacaccctaaacaatgtgcttatttctgtgaaaataagcttcgatct

SEQ ID NO: 38 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagaatgtataagctgtgcgtctatttgtggacaacaccctacacaatgtgcttatttctgtgaaaataagcttcgatct

SEQ ID NO: 39 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtttaagctgtgcgtctatttgtggacaacaccctacacaatgtgcttatttctgtgaaaataagcttcgatct

SEQ ID NO: 40 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaagggacatgcgcagctttctgcaggagtctctcttgcaggaaagaggaaggcaaattttacgaccatcctttgcgagactgtataagctgtgcgtctatttgtggacaacaccctaaacaatgtgcttatttctgtgaaaataagcttcgatct

SEQ ID NO: 41 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaagggacatgcgcagctttctgcaggagtctctcttgcaggaaagagcaaggcaaattttacgaccatcttttgcgagactacataagctgtgcgtctatttgtggacaacaccctaaacaatgtgcttatttctgtgaaaataagcttcgatct

SEQ ID NO: 42 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgattatttctgtgaaaataagcttcgatct

SEQ ID NO: 43 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactatataagctgtgcgtctatttgtggacaacaccctaaacaatgtgcttatttctgtgaaaataagcttcgatct

SEQ ID NO: 44 - DNA, 246 bp, synthetic construct
  source 1..246: mol_type = other DNA
gtggcaatgcgctcatgcccagaggaacaatatcgggatccgcttcttgggacgtgtatgagctgcaagaccatctgtaatcatcaatcccaaaggacatgcgcagctttctgcaggagtctctcttgcaggaaagagcaaggcaaattttacgaccatcctttgcgagactgtataagctgtgcgtctatttgtggtcaacaccctaaacaatgtgcttattactgtgaaaataagcttcgatct

SEQ ID NO: 45 - DNA, 246 bp, synthetic construct
  source 1..246: mol_type = other DNA
gtggcaatgcgctcatgcccagaggaacaatatcgggatccgcttcttgggacgtgtatgagctgcaagaccatctgtaatcatcaatcccgaaggccatgcgcagcattctgcaggagtctctcttgcaggaaagagcaaggcaaattttacgaccatcttttgcgagactgtataagctgtgcgtctatttgtggacaacaccctacacaatgtgcttatttctgtgaaaataagcttcgatct

SEQ ID NO: 46 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcctttgcgagactgtttaagctgtgcgtctatttgtggacaacaccctaaacaatgtgcttatttctgtgaaaataagcttcgatct

SEQ ID NO: 47 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaagcaaattttacgaccatcttttgcgagactgtataagctgtgcgtctatttgtggtcaacactctaaacaatgtgcttatttctgtgaaaataagcttcgatct

SEQ ID NO: 48 - DNA, 129 bp, synthetic construct
  source 1..129: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatct

SEQ ID NO: 49 - DNA, 129 bp, synthetic construct
  source 1..129: mol_type = other DNA
agtctctcttgcaggaaagagcaaggcaaattttacgaccatcttttgcgagtctgtataagctgtgcgtctatttgtggacaacaccctaaacaatgtgcttatttctgtgaaaataagcttcgatct

SEQ ID NO: 50 - DNA, 129 bp, synthetic construct
  source 1..129: mol_type = other DNA
agtctctcttgcaggaaagtgcaaggcaaattttacgaccatcttttgcgagactgtataagctgtgcgtctatttgtggacaacaccctaaacaatgtgcttatttctgtgaaaataagcttcgatct

SEQ ID NO: 51 - DNA, 129 bp, synthetic construct
  source 1..129: mol_type = other DNA
agtctctcttgcaggaaagagcaaggcaaattttacgaccatcttttgctagactgtataagctgtgcgtctatttgtggacaacaccctaaacaatgtgcttatttctgtgaaaataagcttcgatct

SEQ ID NO: 52 - DNA, 129 bp, synthetic construct
  source 1..129: mol_type = other DNA
agtctctcttgcaggaaagagcaaggcgaattttacgaccatcttttgctagactgtataagctgtgcgtctatttgtggacaacaccctaaacaatgtgcttattactgtgaaaataagcttcgatct

SEQ ID NO: 53 - DNA, 129 bp, synthetic construct
  source 1..129: mol_type = other DNA
agtctctcttgcaggaaagagcaaggcaaatttttcgaccatcttttgcgagactgtataagctgtgcgtctatttgtggacaacaccctaaagaatgtgcttatttctgtgaaaataagcttcgatct

SEQ ID NO: 54 - DNA, 129 bp, synthetic construct
  source 1..129: mol_type = other DNA
agtctctcttgcaggaaagagcaaggcaaatttttcgaccatcttttgcgagactgtataagctgtgcgtctatttgtggacaacaccctaaacaatgtgcttatttctgtgaaaataagcttcgatct

SEQ ID NO: 55 - DNA, 129 bp, synthetic construct
  source 1..129: mol_type = other DNA
agtctctcttgcaggaaagagcaaggcaaattttacgaccatcttttgggagactgtataagctgtgcgtctatttgtggacaacaccctaaacaatgtgcttatttctgtgaaaataagcttcgatct

SEQ ID NO: 56 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatctttcgcgagactgtataagctgtgcgtctatttgtggacaacaccctaaacaatgtgcttattcctgtgaaaataagcttcgatct

SEQ ID NO: 57 - DNA, 246 bp, synthetic construct
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcatttgcgagactgtataagctgtgcgtctatctgtggacaacaccctaaacaatgtgcttatttctgtgaaaataagcttcgatct

SEQ ID NO: 58 - DNA, 129 bp, synthetic construct
  source 1..129: mol_type = other DNA
agtctctcttgcaggaaagagcaaggcaaattttacgaccatcttttgcgagactgtataagctgtgcgtctatttgtggacaacaccctaaacaatgtgattatttctgtgaaaataagcttcgatct

SEQ ID NO: 59 - DNA, 129 bp, synthetic construct
  source 1..129: mol_type = other DNA
agtctctcttgcaggaaagagcaaggcgaattttacgaccatcttttgcaagactgtataagctgtgcgtctatttgtggacaacaccctaaacaatgtgcttatttctgtgaaaataagcttcgatct

SEQ ID NO: 60 - DNA, 129 bp, synthetic construct
  source 1..129: mol_type = other DNA
agtctctcttgcaggaaagagcaaggcgaattttacgaccatcttttgcgagactgtataagctgtgcgtctatttgtggacaacaccctaaacaatgtgattatttctgtgaaaataagcttcgatct

SEQ ID NO: 61 - DNA, 129 bp, synthetic construct
  source 1..129: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatct

SEQ ID NO: 62 - DNA, 129 bp, synthetic construct
  source 1..129: mol_type = other DNA
agtctctcttgcaggaaagaggaaggcaaattttacgaccatcttttgcaagactgtataagctgtgcgtctatttgtggacaacaccctaaacaatgtgcttatttctgtgaaaataagcttcgatct

SEQ ID NO: 63 - DNA, 129 bp, synthetic construct
  source 1..129: mol_type = other DNA
agtctctcttgcaggaaagagcgaggcaaattttacgaccatcttttgggagactgtataagctgtgcgtctgtttgtggacaacaccctaaacaatgtgcttatttctgtgaaaataagcttcgatct

SEQ ID NO: 64 - DNA, 129 bp, synthetic construct
  source 1..129: mol_type = other DNA
agtctctcttgcaggaaagagcaaggcgaattttacgaccatcttttgcgagactgtataagctgtgcgtctatttgtggacaacaccctaaacaatgtgatgatttctgtgaaaataagcttcgatct

SEQ ID NO: 65 - DNA, 129 bp, synthetic construct
  source 1..129: mol_type = other DNA
agtctctcttgcaggaaagagcaaggcaaattttatgaccatcttttgcaagactgtataagctgtgcgtctatttgtggacaacaccctaaacaatgtgcttatttctgtgaaaataagcttcgatct

SEQ ID NO: 66 - DNA, 129 bp, synthetic construct
  source 1..129: mol_type = other DNA
agtctctcttgcaggaaagagcaaggcaaattttacgaccatcttttgcaagactgtataaactgtgcgtctatatgtggacaacaccctaaacaatgtgattatttctgtgaaaataagcttcgatct

SEQ ID NO: 67 - DNA, 129 bp, synthetic construct
  source 1..129: mol_type = other DNA
agtctctcttgcaggaaagagcaaggcgaattttacgaccatcttttgcgagactgtataagctgtgcgtctatttgtggacaacaccctaaacaatgtgcttatttctgtgaaaataagcttcgatct

SEQ ID NO: 68 - DNA, 129 bp, synthetic construct
  source 1..129: mol_type = other DNA
agtctctcttgcaggaaagagcaaggcaaattttacgaccatcttttgcaagactgtataagctgtgcgtctatttgtggacaacaccctaaacaatgtgcttatgtctgtgaaaataagcttcgatct

SEQ ID NO: 69 - DNA, 129 bp, synthetic construct
  source 1..129: mol_type = other DNA
agtctctcttgcaggaaagagcaaggcgaattttacgaccatcttttgcgagactgtataagctgtgcgtctatttgtggacgacaccctaaacaatgtgattatttctgtgaaaataagcttcgatct

SEQ ID NO: 70 - DNA, 129 bp, synthetic construct
  source 1..129: mol_type = other DNA
agtctctcttgcaggaaagagcaaggcaaattttacgaccatcttttgcgagactgtatgagctgtgcgtcaatttgtggacaacaccctaaacaatgtgattatttctgtgaaaataagcttcgatct

SEQ ID NO: 71 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 8 aa, synthetic construct
DYKDDDDK

SEQ ID NO: 73 - protein, 231 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 7 aa, synthetic construct
GSGGGGS

SEQ ID NO: 75 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 5 aa, synthetic construct
GSGGS

SEQ ID NO: 77 - protein, 5 aa, synthetic construct
GGGGS

SEQ ID NO: 78 - protein, 10 aa, synthetic construct
GGGGSGGGGS

SEQ ID NO: 79 - protein, 15 aa, synthetic construct
GGGGSGGGGSGGGGS

SEQ ID NO: 80 - protein, 7 aa, synthetic construct
GGGGSSA

SEQ ID NO: 81 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 20 aa, synthetic construct
GGGGSGGGGSGGGGSGGGGS

SEQ ID NO: 85 - protein, 323 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSVAMRSCPEEQYWDPLLGTCMSCKTICNHQSQRTCAAFCRSLSCRKEQGKFYDHLLRDCISCASICGQHPKQCAYFCENKLRS

SEQ ID NO: 86 - protein, 294 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSLSCRKEQGEYYDHLLRDCISCASICGQHPKQCADFCENKLRS

SEQ ID NO: 87 - protein, 30 aa, synthetic construct
EAAAKEAAAKEAAAKEAAAKEAAAKEAAAK

SEQ ID NO: 88 - protein, 293 aa, synthetic construct
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 89 - protein, 231 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 230 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSG

SEQ ID NO: 91 - protein, 25 aa, synthetic construct
GGGGSGGGGSGGGGSGGGGSGGGGS

SEQ ID NO: 92 - protein, 43 aa, synthetic construct
SLSCRKEQGKFYDHPLRDCISCASICGQHPKQCAYFCENKLRS

SEQ ID NO: 93 - protein, 43 aa, synthetic construct
SLSCRKEQGKFYDHLLRECISCASICGQHPTQCAYFCENKLRS

SEQ ID NO: 94 - protein, 43 aa, synthetic construct
SLSCRKEQGKFYDHLLRDCLSCASICGQHPTQCAYFCENKLRS

SEQ ID NO: 95 - protein, 43 aa, synthetic construct; identical to SEQ ID NO: 23

SEQ ID NO: 96 - protein, 43 aa, synthetic construct
SLSCRKEQGKFYDHLLRDYISCASICGQHPKQCAYFCENKLRS

SEQ ID NO: 97 - protein, 43 aa, synthetic construct
SLSCRKEQGKFYDHPLRDCLSCASICGQHPKQCAYFCENKLRS

SEQ ID NO: 98 - protein, 43 aa, synthetic construct
SLSCRKEQSKFYDHLLRDCISCASICGQHSKQCAYFCENKLRS

SEQ ID NO: 99 - protein, 43 aa, synthetic construct
SLSCRKEQGKFYDHLSRDCISCASICGQHPKQCAYSCENKLRS

SEQ ID NO: 100 - protein, 43 aa, synthetic construct
SLSCRKEQGKFYDHHLRDCISCASICGQHPKQCAYFCENKLRS

SEQ ID NO: 101 - protein, 82 aa, synthetic construct
VAMRSCPEEQYWDPLLGTCMSCKTICNHQSQRTCAAFCRSLSCRKEQGKFYDHLLRVCISCASICGQHPKQCAYFCENKLRS

SEQ ID NO: 102 - protein, 82 aa, synthetic construct
VAMRSCPEEQYWDPLLGTCMSCKTICNHQSQRTCAAFCRSLSCRKVQGKFYDHLLRDCISCASICGQHPKQCAYFCENKLRS

SEQ ID NO: 103 - protein, 82 aa, synthetic construct
VAMRSCPEEQYWDPLLGTCMSCKTICNHQSQRTCAAFCRSLSCRKEQGKFYDHLLLDCISCASICGQHPKQCAYFCENKLRS

SEQ ID NO: 104 - protein, 82 aa, synthetic construct
VAMRSCPEEQYWDPLLGTCMSCKTICNHQSQRTCAAFCRSLSCRKEQGEFYDHLLLDCISCASICGQHPKQCAYYCENKLRS

SEQ ID NO: 105 - protein, 82 aa, synthetic construct
VAMRSCPEEQYWDPLLGTCMSCKTICNHQSQRTCAAFCRSLSCRKEQGKFFDHLLRDCISCASICGQHPKECAYFCENKLRS

SEQ ID NO: 106 - protein, 82 aa, synthetic construct
VAMRSCPEEQYWDPLLGTCMSCKTICNHQSQRTCAAFCRSLSCRKEQGKFFDHLLRDCISCASICGQHPKQCAYFCENKLRS

SEQ ID NO: 107 - protein, 82 aa, synthetic construct
VAMRSCPEEQYWDPLLGTCMSCKTICNHQSQRTCAAFCRSLSCRKEQGKFYDHLLGDCISCASICGQHPKQCAYFCENKLRS

SEQ ID NO: 108 - protein, 82 aa, synthetic construct; identical to SEQ ID NO: 7

SEQ ID NO: 109 - protein, 82 aa, synthetic construct
VAMRSCPEEQYWDPLLGTCMSCKTICNHQSQRTCAAFCRSLSCRKEQGEFYDHLLQDCISCASICGQHPKQCAYFCENKLRS

SEQ ID NO: 110 - protein, 82 aa, synthetic construct
VAMRSCPEEQYWDPLLGTCMSCKTICNHQSQRTCAAFCRSLSCRKEQGEFYDHLLRDCISCASICGQHPKQCDYFCENKLRS

SEQ ID NO: 111 - protein, 82 aa, synthetic construct
VAMRSCPEEQYWDPLLGTCMSCKTICNHQSQRTCAAFCRSLSCRKEQGEYYDHLLRDCISCASICGQHPKQCADFCENKLRS

SEQ ID NO: 112 - protein, 82 aa, synthetic construct
VAMRSCPEEQYWDPLLGTCMSCKTICNHQSQRTCAAFCRSLSCRKEEGKFYDHLLQDCISCASICGQHPKQCAYFCENKLRS

SEQ ID NO: 113 - protein, 82 aa, synthetic construct
VAMRSCPEEQYWDPLLGTCMSCKTICNHQSQRTCAAFCRSLSCRKERGKFYDHLLGDCISCASVCGQHPKQCAYFCENKLRS

SEQ ID NO: 114 - protein, 82 aa, synthetic construct
VAMRSCPEEQYWDPLLGTCMSCKTICNHQSQRTCAAFCRSLSCRKEQGEFYDHLLRDCISCASICGQHPKQCDDFCENKLRS

SEQ ID NO: 115 - protein, 82 aa, synthetic construct
VAMRSCPEEQYWDPLLGTCMSCKTICNHQSQRTCAAFCRSLSCRKEQGKFYDHLLQDCISCASICGQHPKQCAYFCENKLRS

SEQ ID NO: 116 - protein, 82 aa, synthetic construct
VAMRSCPEEQYWDPLLGTCMSCKTICNHQSQRTCAAFCRSLSCRKEQGKFYDHLLQDCINCASICGQHPKQCDYFCENKLRS

SEQ ID NO: 117 - protein, 82 aa, synthetic construct
VAMRSCPEEQYWDPLLGTCMSCKTICNHQSQRTCAAFCRSLSCRKEQGEFYDHLLRDCISCASICGQHPKQCAYFCENKLRS

SEQ ID NO: 118 - protein, 82 aa, synthetic construct
VAMRSCPEEQYWDPLLGTCMSCKTICNHQSQRTCAAFCRSLSCRKEQGKFYDHLLQDCISCASICGQHPKQCAYVCENKLRS

SEQ ID NO: 119 - protein, 82 aa, synthetic construct
VAMRSCPEEQYWDPLLGTCMSCKTICNHQSQRTCAAFCRSLSCRKEQGEFYDHLLRDCISCASICGRHPKQCDYFCENKLRS

SEQ ID NO: 120 - protein, 82 aa, synthetic construct
VAMRSCPEEQYWDPLLGTCMSCKTICNHQSQRTCAAFCRSLSCRKEQGKFYDHLLRDCMSCASICGQHPKQCDYFCENKLRS

SEQ ID NO: 121 - protein, 6 aa, synthetic construct
HHHHHH

SEQ ID NO: 122 - protein, 165 aa, synthetic construct
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGKLSADQVALVYST

SEQ ID NO: 123 - protein, 5 aa, synthetic construct
EAAAK

SEQ ID NO: 124 - protein, 10 aa, synthetic construct
EAAAKEAAAK

SEQ ID NO: 125 - protein, 15 aa, synthetic construct
EAAAKEAAAKEAAAK

SEQ ID NO: 126 - protein, 20 aa, synthetic construct
EAAAKEAAAKEAAAKEAAAK

SEQ ID NO: 127 - protein, 25 aa, synthetic construct
EAAAKEAAAKEAAAKEAAAKEAAAK

SEQ ID NO: 128 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 130 - protein, 81 aa, synthetic construct
AMRSCPEEQYWDPLLGTCMSCKTICNHQSQRTCAAFCRSLSCRKEQGKFYDHLLRDCISCASICGQHPKQCAYFCENKLRS

SEQ ID NO: 131 - protein, 106 aa, synthetic construct
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPEL

SEQ ID NO: 132 - protein, 313 aa, synthetic construct
AMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 13 aa, synthetic construct
GGGGSGGGGSAAA

SEQ ID NO: 134 - protein, 231 aa, synthetic construct
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 231 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 230 aa, synthetic construct
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 235 aa, synthetic construct
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 139 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 231 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 142 - protein, 231 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVDGFYPSDIAVEWESNGQPENNYDTTPPVLDSDGSFFLYSDLTVDKSRWQQGNVFSCSVMHEALHNHYTQKSLSLSPG

SEQ ID NO: 143 - protein, 237 aa, synthetic construct
SKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 144 - protein, 237 aa, synthetic construct
SKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 145 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 146 - protein, 45 aa, synthetic construct
DLGPQMLRELQETNAALQDVRELLRQQVREITFLKNTVMECDACG

SEQ ID NO: 147 - protein, 33 aa, synthetic construct
DLQRVKQELLEEVKKELQKVKEEIIEAFVQELR

SEQ ID NO: 148 - protein, 40 aa, synthetic construct
DVQAISSTIQDLQDQVDSLAEVVLQNRRGLDLLTRSGGGS

SEQ ID NO: 149 - protein, 18 aa, synthetic construct
MKWVTFISLLFLFSSAYS

SEQ ID NO: 150 - protein, 22 aa, synthetic construct
MDMRAPAGIFGFLLVLFPGYRS

SEQ ID NO: 151 - protein, 19 aa, synthetic construct
MTRLTVLALLAGLLASSRA

SEQ ID NO: 152 - protein, 19 aa, synthetic construct
MELGLSWIFLLAILKGVQC

SEQ ID NO: 153 - protein, 19 aa, synthetic construct
MELGLRWVFLVAILEGVQC

SEQ ID NO: 154 - protein, 19 aa, synthetic construct
MKHLWFFLLLVAAPRWVLS

SEQ ID NO: 155 - protein, 19 aa, synthetic construct
MDWTWRILFLVAAATGAHS

SEQ ID NO: 156 - protein, 19 aa, synthetic construct
MDWTWRFLFVVAAATGVQS

SEQ ID NO: 157 - protein, 19 aa, synthetic construct
MEFGLSWLFLVAILKGVQC

SEQ ID NO: 158 - protein, 19 aa, synthetic construct
MEFGLSWVFLVALFRGVQC

SEQ ID NO: 159 - protein, 26 aa, synthetic construct
MDLLHKNMKHLWFFLLLVAAPRWVLS

SEQ ID NO: 160 - protein, 22 aa, synthetic construct
MDMRVPAQLLGLLLLWLSGARC

SEQ ID NO: 161 - protein, 22 aa, synthetic construct
MKYLLPTAAAGLLLLAAQPAMA

SEQ ID NO: 162 - protein, 17 aa, synthetic construct
MGVKVLFALICIAVAEA

SEQ ID NO: 163 - protein, 18 aa, synthetic construct
MKWVTFISLLFLFSSAYS

SEQ ID NO: 164 - protein, 18 aa, synthetic construct
MAFLWLLSCWALLGTTFG

SEQ ID NO: 165 - protein, 14 aa, synthetic construct
MQLLSCIALILALV

SEQ ID NO: 166 - protein, 15 aa, synthetic construct
MNLLLILTFVAAAVA

SEQ ID NO: 167 - protein, 281 aa, synthetic construct
SLSCRKEQGEYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 281 aa, synthetic construct
SLSCRKEQGEYYDHLLRDCISCASICGQHPKQCADFCENKLRSGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 281 aa, synthetic construct
SLSCRKEEGKFYDHLLQDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 281 aa, synthetic construct
SLSCRKEEGKFYDHLLQDCISCASICGQHPKQCAYFCENKLRSGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 281 aa, synthetic construct
SLSCRKEQGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 281 aa, synthetic construct
SLSCRKEQGKFYDHLLRDCISCASICGQHPKQCAYFCENKLRSGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 227 aa, synthetic construct
DKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 174 - protein, 232 aa, synthetic construct
DPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 175 - protein, 227 aa, synthetic construct
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 43 aa, synthetic construct
SLSCRKEEGKFYDHLLRDCISCASICGQHPKQCAYFCENKLRS

SEQ ID NO: 178 - protein, 43 aa, synthetic construct
SLSCRKEEGEFYDHLLRDCISCASICGQHPKQCAYFCENKLRS

SEQ ID NO: 179 - protein, 43 aa, synthetic construct
SLSCRKEEGKYYDHLLRDCISCASICGQHPKQCAYFCENKLRS

SEQ ID NO: 180 - protein, 43 aa, synthetic construct
SLSCRKEEGKFYDHLLRDCISCASICGQHPKQCDYFCENKLRS

SEQ ID NO: 181 - protein, 43 aa, synthetic construct
SLSCRKEEGKFYDHLLRDCISCASICGQHPKQCADFCENKLRS

SEQ ID NO: 182 - protein, 43 aa, synthetic construct
SLSCRKEQGEYYDHLLQDCISCASICGQHPKQCAYFCENKLRS

SEQ ID NO: 183 - protein, 43 aa, synthetic construct
SLSCRKEQGKYYDHLLRDCISCASICGQHPKQCAYFCENKLRS

SEQ ID NO: 184 - protein, 43 aa, synthetic construct
SLSCRKEQGKYYDHLLQDCISCASICGQHPKQCAYFCENKLRS

SEQ ID NO: 185 - protein, 43 aa, synthetic construct
SLSCRKEQGKYYDHLLRDCISCASICGQHPKQCDYFCENKLRS

SEQ ID NO: 186 - protein, 43 aa, synthetic construct
SLSCRKEQGKYYDHLLRDCISCASICGQHPKQCADFCENKLRS

SEQ ID NO: 187 - protein, 43 aa, synthetic construct
SLSCRKEQGKFYDHLLQDCISCASICGQHPKQCDYFCENKLRS

SEQ ID NO: 188 - protein, 43 aa, synthetic construct
SLSCRKEQGKFYDHLLQDCISCASICGQHPKQCADFCENKLRS

SEQ ID NO: 189 - protein, 43 aa, synthetic construct
SLSCRKEQGKFYDHLLRDCISCASICGQHPKQCDDFCENKLRS

SEQ ID NO: 190 - protein, 43 aa, synthetic construct
SLSCRKEQGKFYDHLLRDCISCASICGQHPKQCADFCENKLRS

SEQ ID NO: 191 - protein, 43 aa, synthetic construct
SLSCRKEQGEYYDHLLRDCISCASICGQHPKQCAYFCENKLRS

SEQ ID NO: 192 - protein, 43 aa, synthetic construct
SLSCRKEQGEFYDHLLRDCISCASICGQHPKQCADFCENKLRS

SEQ ID NO: 193 - protein, 227 aa, synthetic construct; identical to SEQ ID NO: 173

SEQ ID NO: 194 - protein, 12 aa, synthetic construct
GSGGGGSGGGGS

SEQ ID NO: 195 - protein, 65 aa, synthetic construct
GGGGSGGGGSGGGGSGGGGSGGGGSGGGGSGGGGSGGGGSGGGGSGGGGSGGGGSGGGGSGGGGS

SEQ ID NO: 196 - protein, 577 aa, synthetic construct
SLSCRKEQGKFYDHLLRDCISCASICGQHPKQCAYFCENKLRSGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 281 aa, synthetic construct; identical to SEQ ID NO: 171

SEQ ID NO: 198 - protein, 344 aa, synthetic construct
SLSCRKEQGKFYDHLLRDCISCASICGQHPKQCAYFCENKLRSGGGGSGGGGSGGGGSGGGGSSLSCRKEQGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 577 aa, synthetic construct
SLSCRKEQGEYYDHLLRDCISCASICGQHPKQCADFCENKLRSGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 281 aa, synthetic construct; identical to SEQ ID NO: 167

SEQ ID NO: 201 - protein, 344 aa, synthetic construct
SLSCRKEQGEYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSLSCRKEQGEYYDHLLRDCISCASICGQHPKQCADFCENKLRS

SEQ ID NO: 202 - protein, 344 aa, synthetic construct
SLSCRKEQGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSLSCRKEQGKFYDHLLRDCISCASICGQHPKQCAYFCENKLRS

SEQ ID NO: 203 - protein, 616 aa, synthetic construct
VAMRSCPEEQYWDPLLGTCMSCKTICNHQSQRTCAAFCRSLSCRKEQGKFYDHLLRDCISCASICGQHPKQCAYFCENKLRSGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 313 aa, synthetic construct; identical to SEQ ID NO: 132

SEQ ID NO: 205 - protein, 640 aa, synthetic construct
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 206 - protein, 333 aa, synthetic construct
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - DNA, 1731 bp, synthetic construct
  source 1..1731: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 208 - DNA, 843 bp, synthetic construct
  source 1..843: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 209 - DNA, 1032 bp, synthetic construct
  source 1..1032: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctgggggtggcggatcaggaggtggcggttcaggcggaggaggttctgggggtgggggttcttcattgtcctgcagaaaggaacaggggaaattttacgatcacttgcttagagattgtataagctgcgcgagcatttgcgggcaacaccctaaacagtgtgcgtatttctgcgagaataaactccggtctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 210 - DNA, 1731 bp, synthetic construct
  source 1..1731: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatctggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 211 - DNA, 843 bp, synthetic construct
  source 1..843: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 212 - DNA, 1032 bp, synthetic construct
  source 1..1032: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggggggggtggcggatcaggaggtggcggttcaggcggaggaggttctgggggtgggggttcttcattgtcctgcagaaaggaacagggggagtattacgatcacttgcttagagattgtataagctgcgcgagcatttgcgggcaacaccctaaacagtgtgcggatttctgcgagaataaactccggtct

SEQ ID NO: 213 - DNA, 1032 bp, synthetic construct
  source 1..1032: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggggggggtggcggatcaggaggtggcggttcaggcggaggaggttctgggggtgggggttcttcattgtcctgcagaaaggaacaggggaaattttacgatcacttgcttagagattgtataagctgcgcgagcatttgcgggcaacaccctaaacagtgtgcgtatttctgcgagaataaactccggtct

SEQ ID NO: 214 - DNA, 1848 bp, synthetic construct
  source 1..1848: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 215 - DNA, 939 bp, synthetic construct
  source 1..939: mol_type = other DNA
gcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctgagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccgagctccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccggggaag

SEQ ID NO: 216 - DNA, 1920 bp, synthetic construct
  source 1..1920: mol_type = other DNA
agccgtgtggaccaggaggagcgctttccacagggcctgtggacgggggtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctcccgtgaacctgccacccgagctgggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 217 - DNA, 999 bp, synthetic construct
  source 1..999: mol_type = other DNA
agccgtgtggaccaggaggagcgctttccacagggcctgtggacgggggtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctcccgtgaacctgccacccgagctggacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccgagctccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccggggaag

SEQ ID NO: 218 - protein, 522 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 222 aa, synthetic construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 220 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 221 - protein, 226 aa, synthetic construct
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 222 - protein, 281 aa, synthetic construct
SLSCRKEEGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 281 aa, synthetic construct
SLSCRKEEGEFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 281 aa, synthetic construct
SLSCRKEEGKYYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 281 aa, synthetic construct
SLSCRKEEGKFYDHLLRDCISCASICGQHPKQCDYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 281 aa, synthetic construct
SLSCRKEEGKFYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 281 aa, synthetic construct
SLSCRKEQGEYYDHLLQDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 281 aa, synthetic construct
SLSCRKEQGKYYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 281 aa, synthetic construct
SLSCRKEQGKYYDHLLQDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 230 - protein, 281 aa, synthetic construct
SLSCRKEQGKYYDHLLRDCISCASICGQHPKQCDYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 231 - protein, 281 aa, synthetic construct
SLSCRKEQGKYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 281 aa, synthetic construct
SLSCRKEQGKFYDHLLQDCISCASICGQHPKQCDYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 233 - protein, 281 aa, synthetic construct
SLSCRKEQGKFYDHLLQDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 234 - protein, 281 aa, synthetic construct
SLSCRKEQGKFYDHLLRDCISCASICGQHPKQCDDFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 235 - protein, 281 aa, synthetic construct
SLSCRKEQGKFYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 236 - protein, 281 aa, synthetic construct
SLSCRKEQGEYYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 237 - protein, 281 aa, synthetic construct
SLSCRKEQGEFYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 238 - protein, 5 aa, synthetic construct
EPKSS

SEQ ID NO: 239 - protein, 5 aa, synthetic construct
EPKSC

SEQ ID NO: 240 - protein, 333 aa, synthetic construct
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 241 - protein, 333 aa, synthetic construct; identical to SEQ ID NO: 206

(19) United States
(12) Patent Application Publication
(10) Pub. No.: US 2026/0297163 A1
(43) Pub. Date: Oct. 1, 2026

(54) Formulations of TACI-Fc Fusion Immunomodulatory Protein and Uses Thereof

(71) Applicant: Alpine Immune Sciences, Inc., Seattle, WA (US)

(72) Inventors: Julee Alaina Floyd, Seattle, WA (US); James Barnard, Orange, CA (US); Yan Brodsky, Redmond, WA (US); Wayne Richard Gombotz, Kenmore, WA (US); Bruce Arthur Kerwin, Bainbridge Island, WA (US); Amanda Sarah Ann Rohrbach, Duvall, WA (US); Michael Daryl Salema, Lakewood, WA (US)

(73) Assignee: Alpine Immune Sciences, Inc., Seattle, WA (US)

(21) Appl. No.: 19/631,876

(22) Filed: Mar. 27, 2026

Related U.S. Application Data
(60) Provisional application No. 63/779,927, filed on Mar. 28, 2025.

(51) Int. Cl. C07K 14/705; A61K 9/08; A61K 38/00; A61K 47/02; A61K 47/10; A61K 47/12; A61K 47/18; A61K 47/22; A61K 47/26

(52) U.S. Cl. CPC C07K 14/70578; A61K 9/08; A61K 47/02; A61K 47/10; A61K 47/12; A61K 47/183; A61K 47/22; A61K 47/26; A61K 38/00; C07K 2319/30

(57) ABSTRACT

Provided herein are formulations of immunomodulatory TACI-Fc fusion proteins, and methods and uses of the provided formulations for treating a variety of immunological diseases, disorders or conditions, such as B cell-mediated diseases, disorder or conditions. The formulations provided herein provide improved stability of the TACI-Fc fusion protein for long-term storage.

### CROSS-REFERENCE TO RELATED APPLICATIONS

This application claims the benefit of priority to U.S. Provisional Application No. 63/779,927, entitled “FORMULATIONS OF TACI-FC FUSION IMMUNOMODULATORY PROTEIN AND USES THEREOF,” filed Mar. 28, 2025, the contents of which are incorporated herein by reference in their entirety for all purposes.

### INCORPORATION BY REFERENCE OF SEQUENCE LISTING

The application contains a Sequence Listing which has been submitted electronically in .XML format and is hereby incorporated by reference in its entirety. Said .XML copy, created on Mar. 26, 2026, is named “01245-0082-00US_xml” and is 254,105 bytes in size. The sequence listing contained in this .XML file is part of the specification and is hereby incorporated by reference herein in its entirety

### FIELD

The present disclosure provides formulations of immunomodulatory TACI-Fc fusion proteins. The formulations containing an immunomodulatory TACI-Fc fusion protein are for use in methods and uses to provide therapeutic utility for a variety of immunological diseases, disorders or conditions, such as B cell-mediated diseases, disorder or conditions. The formulations provided herein provide improved stability of the TACI-Fc fusion protein for long-term storage.

### BACKGROUND

Modulation of the immune response by intervening in processes involving interactions between soluble ligands and their receptors is of increasing medical interest. B cell activating factor (BAFF) and a proliferation inducing ligand (APRIL) are TNF superfamily members that bind TACI (transmembrane activator and CAML interactor), BCMA (B cell maturation antigen), and/or BAFF-R on B cells and together support B cell development, differentiation, and survival. The neutralization of BAFF, APRIL and/or BAFF-R by TACI-Fc fusion proteins reduces B cell survival and function including antibody production, and thereby can be used to modulate the immune response and particularly B cell responses, such as for treatment for various autoimmune and inflammatory conditions. Improved strategies are needed to improve the therapeutic effectiveness of TACI-Fc fusion proteins. Provided are embodiments that meet such needs.

### SUMMARY

Embodiments herein include but are not limited to the following enumerated embodiments. Additional embodiments herein are also described throughout the application.
1. A formulation comprising a TACI-Fc fusion protein, comprising:
a. at or about 50 mM to about 200 mM of a tonicifying agent, wherein the tonicifying agent comprises arginine;
b. an acetate buffer; and
c. at or about 0.002% to about 1.0% (w/v) of a non-ionic surfactant;
wherein the formulation comprises:
a TACI-Fc fusion protein concentration of at or about 140 mg/mL to about 200 mg/mL; and
a formulation pH of at or about 4.4 to about 5.1.
2. A formulation comprising a TACI-Fc fusion protein, comprising:
a. at or about 1% to about 4% (w/v) proline;
b. an acetate buffer; and
c. at or about 0.002% to about 1.0% (w/v) of a non-ionic surfactant;
a TACI-Fc fusion protein concentration of at or about 140 mg/mL to about 200 mg/mL; and
a formulation pH of at or about 4.7 to about 5.7.
3 A formulation comprising a TACI-Fc fusion protein, comprising:
a. at or about 1% to about 4% (w/v) proline;
b. an acetate buffer;
c. at or about 0.002% to about 1.0% (w/v) of a non-ionic surfactant; and
d. a salt; wherein
a TACI-Fc fusion protein concentration of at or about 140 mg/mL to about 200 mg/mL; and
a formulation pH of at or about 4.4 to about 5.1.
4. A formulation comprising a TACI-Fc fusion protein, comprising:
a. water and a buffering agent that provides a formulation pH of at or about 4.3 to about 5.5;
b. a tonicifying agent having a positive charge at the formulation pH;
c. a non-ionic surfactant; and
d. a TACI-Fc fusion protein at a concentration of at or about 100 mg/mL to about 200 mg/mL.
5. The formulation of embodiments 1 or 4, wherein the tonicifying agent is present in the formulation at a concentration of at or about 50 mM to about 200 mM, 70 mM to about 190 mM; at or about 135 mM to about 165 mM; at or about 145 M to about 150 mM; at or about 140 mM, at or about 145 mM, at or about 150 mM, or at or about 155 mM.
6. The formulation of any one of embodiments 1-5 having a tonicity of an isotonic tonicity, of at or about 250 to about 350 mOsm/Kg, or of at or about 250 mOsm/Kg to about 600 mOsm/Kg.
7. The formulation of any one of embodiments 1-6, comprising at or about 40 mM to about 150 mM sodium chloride.
8. The formulation of any one of embodiments 1 and 3-7, wherein the formulation pH is at or about 4.5 to about 5.0; at or about 4.5 to about 4.8; at or about 4.6 at or about 4.7, or at or about 4.8.
9. The formulation of any one of embodiments 1-8, wherein TACI-Fc fusion protein concentration is at or about 150 mg/mL, at or about 160 mg/mL, at or about 170 mg/mL, at or about 175 mg/mL, at or about 180 mg/mL, at or about 190 mg/mL, or at or about 200 mg/mL.
10. The formulation of any one of embodiments 1-9, comprising from at or about 1 mM to about 30 mM, from about 3 mM to about 15 mM, from at or about 4 mM to about 20 mM, from at or about 5 mM to about 10 mM, from at or about 8 mM to about 25 mM, from at or about 10 mM to about 20 mM, from at or about 10 mM to about 30 mM, from at or about 7 mM to about 25 mM acetate, or from at or about 15 mM to about 25 mM acetate as a component of the acetate buffer.
11. The formulation of any one of embodiments 1-10, wherein the non-ionic surfactant is a polysorbate (polysorbate 80), or is a poloxamer polyether (poloxamer 188).
12. The formulation of any one of embodiments 1-11, comprising at or about 0.002% to about 0.2% (w/v); at or about 0.01% to about 0.04% (w/v); or at or about 0.02% (w/v) of the non-ionic surfactant, wherein the non-ionic surfactant is polysorbate 80.
13. The formulation of any one of embodiments 1-11, comprising 0.01% to about 1.0% (w/v); at or about 0.05% to about 0.2% (w/v); or at or about 0.1% (w/v) of the non-ionic surfactant, wherein the non-ionic surfactant is poloxamer 188.
14. A formulation comprising a TACI-Fc fusion protein, comprising:
a. at or about 135 mM to about 165 mM arginine;
b. an acetate buffer; and
c. at or about 0.05% to about 0.2% (w/v) poloxamer 188 (P188); wherein the TACI-Fc fusion protein is at a concentration of at or about 160 mg/mL to about 190 mg/mL; and the formulation has a formulation pH of at or about 4.6 to about 4.8.
15. A formulation comprising a TACI-Fc fusion protein, comprising:
a. at or about 140 mM to about 150 mM arginine;
b. an acetate buffer; and
c. at or about 0.1% (w/v) poloxamer 188 (P188); wherein
the TACI-Fc fusion protein is at a concentration of at or about 175 mg/mL; and the formulation has a formulation pH of at or about 4.7.
16. A formulation comprising a TACI-Fc fusion protein, comprising:
a. at or about 140 mM to about 150 mM arginine;
b. an acetate buffer; and
c. at or about 0.01% to about 0.04% (w/v) polysorbate 80 (PS80); wherein
the TACI-Fc fusion protein is at a concentration of at or about 175 mg/mL; and the formulation has a formulation pH of at or about 4.7.
17. A formulation comprising a TACI-Fc fusion protein, comprising:
a. at or about 3.0% (w/v) proline;
b. an acetate buffer;
c. at or about 0.05% (w/v) to about 0.2% (w/v) poloxamer 188; and
d. at or about 50 mM sodium chloride; wherein
the TACI-Fc fusion protein is at a concentration of at or about 175 mg/mL; and the formulation has a formulation pH of at or about 4.7.
18. A formulation comprising a TACI-Fc fusion protein, comprising:
a. at or about 3.0% (w/v) proline;
b. an acetate buffer; and
c. at or about 0.01% to about 0.04% (w/v) polysorbate 80 (PS80); wherein
the TACI-Fc fusion protein is at a concentration of at or about 175 mg/mL; and the formulation has a formulation pH of at or about 5.2.
19. The formulation of any one of embodiments 1, 2, 4-11, 13-15 and 17, wherein the formulation does not comprise a polysorbate surfactant.
20. The formulation of any one of embodiments 1-19, wherein the TACI-Fc fusion protein comprises (i) a TACI polypeptide sequence comprising the amino acid sequence of SEQ ID NO: 26; and (ii) an immunoglobulin Fc region.
21. The formulation of embodiment 20, wherein the immunoglobulin Fc region comprises the amino acid sequence of any one of SEQ ID NOS: 73, 75, 83, 136, 175, 176, or 221.
22. The formulation of any one of embodiments 1-19, wherein the TACI-Fc fusion protein comprises the amino acid sequence of SEQ ID NO: 168.
23. The formulation of any one of embodiments 1-19, wherein the TACI-Fc fusion protein comprises the amino acid sequence of SEQ ID NO: 167.
24. The formulation of any one of embodiments 1-23, wherein the TACI-Fc fusion protein is a homodimer comprising two identical copies of the TACI-Fc fusion protein.
25. The formulation of any one of embodiments 1-19 wherein the TACI-Fc fusion protein is a homodimer comprising two identical copies of the TACI-Fc fusion protein set forth in SEQ ID NO:168 linked by a covalent disulfide bond.
26. The formulation of any one of embodiments 1-19 wherein the TACI-Fc fusion protein is a homodimer comprising two identical copies of the TACI-Fc fusion protein set forth in SEQ ID NO:167 linked by a covalent disulfide bond.
27. The formulation of any one of embodiments 1-26, wherein the TACI-Fc fusion protein comprises a variant TACI polypeptide, and/or the TACI-Fc fusion protein has an isoelectric point (pI) of at or about 7.0 to about 7.8, at or about 7.0 to about 7.7, at or about 7.0 to about 7.6, at or about 7.0 to about 7.5, at or about 7.0 to about 7.3, at or about 7.1 to about 7.5, at or about 7.1 to about 7.3, or about 7.2.
28. The formulation of any one of embodiments 1-27, that is a liquid formulation that has not been lyophilized and is not lyophilized prior to administration to a subject.
29. The formulation of any one of embodiments 1-28, wherein, after storage for 12 months in liquid form at a storage temperature of at or about 2° C. to about 8° C., optionally at or about 5° C., and as measured by size exclusion chromatography (SEC) and as expressed in terms of area % under a curve that includes the TACI-Fc fusion protein in a form of a homodimer comprising two identical copies of the TACI-Fc fusion protein linked by a covalent disulfide bond as a main species, wherein:
the main species is at least 95%, at least 98%, or at least 99% of all observed species under the curve;
less than 5%, less than 2%, or less than 1% of species under the curve have a size greater than a size of the main species;
less than 5%, less than 2%, or less than 1% of species under the curve have a size less than a size of the main species;
the loss of the main species is less than 1%;
the increase in species under the curve having a size greater than a size of the main species is less than 1%; and/or
the increase in species under the curve having a size less than a size of the main species is less than 1%.
30. The formulation of any one of embodiments 1-29, wherein the formulation is a liquid formulation having a viscosity of at or about 3 cP to about 15 cP at about 25° C. as assessed using a rheometer or a cone and plate viscometer.
31. The formulation of any one of embodiments 1-30, wherein the formulation is a liquid formulation comprising less than about 6,000 particles/mL having a particle size diameter of ≥10 μm; and/or comprising less than about 600 particles/mL having a particle size diameter of ≥25 μm, after storage for 12 months in liquid form at a storage temperature of at or about 2° C. to about 8° C., optionally at or about 5° C.
32. An article of manufacture comprising the formulation of any one of embodiments 1-31 in a container, for example in a vial or a syringe barrel.
33. A kit comprising the formulation of any one of embodiments 1-31 or the article of manufacture of embodiment 32, and instructions for use.
34. A method of manufacturing the formulation of any one of embodiments 1-31, the method comprising combining the TACI-Fc fusion protein and the components (a), (b), and (c).
35. A method for reducing degradation and/or aggregation of a TACI-Fc fusion protein homodimer in a liquid pharmaceutical formulation comprising a TACI-Fc fusion protein at a concentration of at least 160 mg/mL, comprising combining the TACI-Fc fusion protein and the components (a), (b), and (c) of any one of the formulations of embodiments 1-31.
36. A method of reducing an immune response in a subject, comprising administering the formulation of any one of embodiments 1-31 to a subject in need thereof.
37. The formulation of any one of embodiments 1-31 for use in reducing an immune response in a subject.
38. Use of formulation of any one of embodiments 1-31 in the manufacture of a medicament for reducing an immune response in a subject.
39. The method of embodiment 36, the formulation for use of embodiment 37, or the use of embodiment 38, wherein the immune response is a B cell immune response, wherein reducing the immune response reduces or inhibits B cell maturation, differentiation and/or proliferation; and/or wherein reducing the immune response reduces circulating levels of APRIL, BAFF or an APRIL/BAFF heterotrimer in the subject.
40. The method of embodiment 36 or 39, the formulation for use of embodiment 37 or 39, or the use of embodiment 38 or 39, wherein reducing the immune response treats a disease, disorder or condition in the subject.
41. A method of treating a disease, disorder or condition in a subject, comprising administering a formulation of any one of embodiments 1-31 to a subject in need thereof.
42. The formulation of any one of embodiments 1-31 for use in treating a disease, disorder or condition in a subject.
43. Use of the formulation of any one of embodiments 1-31 in the manufacture of a medicament for treating a disease, disorder or condition in a subject.
44. The method of embodiment 40 or 41, the formulation for use of embodiment 40 or 42, or the use of embodiment 40 or 43, wherein the disease, disorder or condition is an autoimmune disease or an inflammatory condition.
45. The method of embodiment 40 or 41, the formulation for use of embodiment 40 or 42, or the use of embodiment 40 or 43, wherein the disease, disorder or condition is a B cell cancer, an antibody-mediated pathology, a renal disease, a graft rejection, graft versus host disease, or a viral infection.
46. The method of embodiment 40 or 41, the formulation for use of embodiment 40 or 42, or the use of embodiment 40 or 43, wherein the disease, disorder or condition is selected from the group consisting of systemic lupus erythematosus (SLE), lupus nephritis, cutaneous lupus erythematosus, Sjögren's syndrome, scleroderma (systemic sclerosis), multiple sclerosis, diabetes (e.g. Type I diabetes), polymyositis, primary biliary cirrhosis, IgG4-related disease, IgA nephropathy, IgA vasculitis, ANCA vasculitis (microscopic polyangiitis, granulomatosis with polyangiitis [Wegener's granulomatosis], eosinophilic granulomatosis with polyangiitis [Churg-Strauss]) cryoglobulinemia, cold agglutinin or warm agglutinin disease, immune thrombocytopenic purpura, optic neuritis, amyloidosis, antiphospholipid antibody syndrome (APS), autoimmune polyglandular syndrome type II (APS II), autoimmune thyroid disease (AITD), Graves' disease, autoimmune adrenalitis, pemphigus vulgaris, bullous pemphigoid, myasthenia gravis, graft versus host disease (GVHD), transplantation, rheumatoid arthritis, acute lupus nephritis, amyotrophic lateral sclerosis, neuromyelitis optica, transverse myelitis, Rasmussen's encephalitis, CNS autoimmunity, Guillain-Barre syndrome, chronic inflammatory demyelinating polyneuropathy, neurocystercercosis, sarcoidosis, antiphospholipid antibody syndrome, IgG4-related disease, Hashimoto's thyroiditis, immune thrombocytopenia, Addison's Disease, and dermatomyositis.
47. The method of embodiment 40 or 41, the formulation for use of embodiment 40 or 42, or the use of embodiment 40 or 43, wherein the disease or disorder is autoantibody-associated glomerular disease.
48. The method, the formulation for use, or the use of embodiment 46 or 47, wherein the disease, disorder, or condition is immunoglobulin (Ig) A nephropathy (IgAN), lupus nephritis (LN), primary membranous nephropathy (pMN), myasthenia gravis, or renal anti-neutrophil cytoplasmic antibody (ANCA)-associated vasculitis (AAV).
49. The method of embodiment 40 or 41, the formulation for use of embodiment 40 or 42, or the use of embodiment 40 or 43, wherein the disease, disorder or condition is immune thrombocytopenia, autoimmune hemolytic anemia, or cold agglutinin disease.
50. The method of embodiment 40 or 41, the formulation for use of embodiment 40 or 42, or the use of embodiment 40 or 43, wherein the disease, disorder or condition is a B cell cancer, optionally wherein the cancer is myeloma.

Provided herein are also, among other embodiments, a formulation containing a TACI-Fc fusion protein, about 50 mM to about 200 mM of a tonicifying agent comprising arginine, or about 1.5% to about 4% (w/v) arginine hydrochloride (Arg-HCl); about 5 mM to about 30 mM of an acetate; and about 0.01% to about 0.2% (w/v) of a surfactant, optionally a non-ionic surfactant; wherein the concentration of the TACI-Fc fusion protein in the formulation is between about 150 mg/mL and about 200 mg/mL; and the formulation pH is at or about 4.4 to about 5.1.

In some of any embodiments, the formulation contains at or about 2.7% to 3.5% (w/v) arginine hydrochloride; or wherein the formulation contains arginine hydrochloride at a concentration of at or about 3.1% (w/v); or wherein the formulation contains about 135 mM to about 165 mM of a tonicifying agent comprising arginine.

In some of any embodiments, the formulation containing a TACI-Fc fusion protein, contains about 1.5% to about 4% (w/v) proline; about 5 mM to about 30 mM of an acetate; about 0.01% to about 0.2% (w/v) of surfactant, optionally a non-ionic surfactant; and a salt; wherein the concentration of the TACI-Fc fusion protein in the composition is between about 150 mg/mL and about 200 mg/mL; and the formulation pH is at or about 4.4 to about 5.1.

In some of any embodiments, the formulation containing a TACI-Fc fusion protein, contains about 1.5% to about 4% (w/v) proline; about 5 mM to about 30 mM of an acetate; and a salt; wherein the concentration of the TACI-Fc fusion protein in the composition is between about 150 mg/mL and about 200 mg/mL; and the formulation pH is at or about 4.4 to about 5.1. In some of any embodiments, the proline in the formulation is L-proline. In some of any embodiments, the proline is at a concentration of at or about 2% or at or about 3% (w/v). In some of any embodiments, the formulation contains a salt, wherein the salt is sodium chloride (NaCl). In some of any embodiments, the formulation contains between about 40 mM and 100 mM NaCl. In some of any embodiments, the NaCl is at a concentration of 50 mM.

In some of any embodiments, the formulation pH is at or about 4.5 to about 5.0. In some of any embodiments, the formulation pH is at or about 4.5 to about 4.8. In some of any embodiments, the formulation pH is at or about 4.7. In some of any embodiments, the formulation pH is at or about 4.6.

In some of any embodiments, the formulation contains at or about 10 mM acetate. In some of any embodiments, the formulation contains at or about 20 mM acetate. In some of any embodiments, the acetate is sodium acetate.

In some of any embodiments, the concentration of the TACI-Fc fusion protein in the formulation is at or about 140 mg/mL, at or about 160 mg/mL, at or about 165 mg/mL, at or about 170 mg/mL, at or about 175 mg/mL, at or about 180 mg/mL, or at or about 190 mg/mL, at or about 200 mg/mL, at or about 210 mg/mL, at or about 220 mg/mL, or at or about 230 mg/mL. In some of any embodiments, the concentration of the TACI-Fc fusion protein in the formulation is at or about 160 mg/mL. In some of any embodiments, the concentration of the TACI-Fc fusion protein in the formulation is at or about 175 mg/mL.

In some of any embodiments, the formulation contains at or about 0.015% of surfactant. In some of any embodiments, the formulation contains at or about 0.02% of surfactant. In some of any embodiments, the formulation contains at or about 0.04% to 0.16% of surfactant. In some of any embodiments, the formulation contains at or about 0.1% of surfactant. In some of any embodiments, the formulation contains a surfactant that is a non-ionic surfactant. In some of any embodiments, the formulation contains a surfactant that is a poloxamer. In some of any embodiments, the formulation contains a surfactant that is a poloxamer 188. In some of any embodiments, the formulation contains a surfactant that is a polysorbate 80. In some of any embodiments, the surfactant is not a polysorbate. In some of any embodiments, the formulation does not comprise a polysorbate surfactant.

In some of any embodiments, the formulation containing a TACI-Fc fusion protein, contains about at or about 3.1% (w/v) arginine hydrochloride (Arg-HCl) or contains about 135 mM to about 165 mM or about 140 mM to about 150 mM of a tonicifying agent comprising arginine; at or about 10 mM sodium acetate; and at or about 0.1% (w/v) poloxamer 188 (P188); wherein the concentration of the TACI-Fc fusion protein in the composition is about 160 mg/mL; and the formulation pH is at or about 4.7.

In some of any embodiments, the formulation containing a TACI-Fc fusion protein, contains about at or about 3.1% (w/v) arginine hydrochloride (Arg-HCl) or contains about 135 mM to about 165 mM or about 140 mM to about 150 mM of a tonicifying agent comprising arginine; at or about 10 mM sodium acetate; and at or about 0.1% (w/v) poloxamer 188 (P188); wherein the concentration of the TACI-Fc fusion protein in the composition is about 175 mg/mL; and the formulation pH is at or about 4.7.

In some of any embodiments, the formulation containing a TACI-Fc fusion protein, contains at or about 3.1% (w/v) arginine hydrochloride (Arg-HCl) or contains about 135 mM to about 165 mM or about 140 mM to about 150 mM of a tonicifying agent comprising arginine; at or about 10 mM sodium acetate; and at or about 0.02% (w/v) polysorbate 80 (PS80); wherein the concentration of the TACI-Fc fusion protein in the composition is about 160 mg/mL; and the formulation pH is at or about 4.7.

In some of any embodiments, the formulation containing a TACI-Fc fusion protein, contains at or about 3.1% (w/v) arginine hydrochloride (Arg-HCl) or contains about 135 mM to about 165 mM or about 140 mM to about 150 mM of a tonicifying agent comprising arginine; at or about 10 mM sodium acetate; and at or about 0.02% (w/v) polysorbate 80 (PS80); wherein the concentration of the TACI-Fc fusion protein in the composition is about 175 mg/mL; and the formulation pH is at or about 4.7.

In some of any embodiments, the formulation containing a TACI-Fc fusion protein, contains at or about 2.0% or at or about 3.0% (w/v) proline; at or about 10 mM sodium acetate; at or about 0.1% (w/v) poloxamer 188; and at or about 50 mM sodium chloride; wherein the concentration of the TACI-Fc fusion protein in the composition is about 160 mg/mL; and the formulation pH is at or about 4.7.

In some of any embodiments, the formulation containing a TACI-Fc fusion protein, contains at or about 2.0% or at or about 3.0% (w/v) proline; at or about 10 mM sodium acetate; at or about 0.1% (w/v) poloxamer 188; and at or about 50 mM sodium chloride; wherein the concentration of the TACI-Fc fusion protein in the composition is about 175 mg/mL; and the formulation pH is at or about 4.7.

In some of any embodiments, the formulation containing a TACI-Fc fusion protein, contains at least 90% of the TACI-Fc fusion protein in the main (homodimeric) species. In some of any embodiments, at least 95% of the TACI-Fc fusion protein is in the main species. In some of any embodiments, at least 98% of the TACI-Fc fusion protein is in the main species. In some of any embodiments, at least 99% of the TACI-Fc fusion protein is in the main species.

In some of any embodiments, at least 90% of the TACI-Fc protein in the formulation is the main TACI-Fc protein species containing two polypeptide chains of SEQ ID NO. 167 linked by a covalent bond. In some of any embodiments, at least 95% of the TACI-Fc in protein the formulation is the main TACI-Fc protein species containing two polypeptide chains of SEQ ID NO. 167 linked by a covalent bond. In some of any embodiments, at least 98% of the TACI-Fc protein in the formulation is the main TACI-Fc protein species containing two polypeptide chains of SEQ ID NO. 167 linked by a covalent bond.

In some of any embodiments, the molecular weight of the main species of a TACI-Fc fusion protein is at or about 62.6 kDa to at or about 116.4 kDa. In some of any embodiments, the molecular weight of the main species of a TACI-Fc fusion protein is at or about 62.6 kDa.

In some of any embodiments, less than 5% of the TACI-Fc fusion protein in the formulation is a high molecular weight (HMW) species. In some of any embodiments, the molecular weight of the HMW species of a TACI-Fc fusion protein is greater than the molecular weight of the corresponding main species of the TACI-Fc fusion protein. In some of any embodiments, the molecular weight of the HMW species of a TACI-Fc fusion protein is at least 93.9 kDa.

In some of any embodiments, less than 5% of the TACI-Fc fusion protein in the formulation is a low molecular weight (LMW) species. In some of any embodiments, the molecular weight of the LMW species of a TACI-Fc fusion protein is less than 62.6 kDa.

In some of any embodiments, the combination of the low molecular weight species and high molecular weight species is less than 5% of the TACI-Fc fusion protein in the formulation.

In some of any embodiments, the TACI-Fc protein species is assessed at about 2° C. to about 8° C., optionally at or about 5° C. In some of any embodiments, the TACI-Fc protein species is assessed at about 12° C. to about 18° C., optionally at or about 15° C. In some of any embodiments, the TACI-Fc species is assessed at about 18° C. to about 22° C., optionally at or about 20° C. In some of any embodiments, the TACI-Fc species is assessed about 22° C. to about 28° C., optionally at or about 25° C. In some of any embodiments, the TACI-Fc species is assessed at about 28° C. to about 32° C., such as at or about 30° C.

In some of any embodiments, the main species, high molecular weight species, and/or low molecular weight species of the TACI-Fc fusion protein are assessed by size exclusion chromatography (SEC), Reduced Capillary electrophoresis sodium dodecyl sulfate (rCE-SDS), or Imaged capillary isoelectric focusing (icIEF). In some of any embodiments, the main species, high molecular weight species, and/or low molecular weight species of the TACI-Fc fusion protein are assessed by size exclusion chromatography (SEC).

In some of any embodiments, upon storage the loss of the main species of the TACI-Fc fusion protein in the formulation is less than 1% per week; and/or the increase in the amount of low molecular weight (LMW) species of the TACI-Fc fusion protein in the formulation is less than about 0.03% per week; and/or the increase in the amount of high molecular weight (HMW) species of the TACI-Fc fusion protein in the formulation is less than 0.9% per week.

In some of any embodiments, the loss of the main species of the TACI-Fc fusion protein in the formulation is less than 0.1% per week. In some of any embodiments, the increase in the amount of low molecular weight (LMW) species of the TACI-Fc fusion protein in the formulation is less than 0.1% per week. In some of any embodiments, the increase in the amount of high molecular weight (HMW) species of the TACI-Fc fusion protein in the formulation is less than 0.1% per week.

In some of any embodiments, one or more of the viscosity of the formulation is less than about 15 cP; the concentration of particles with average particle size of less than about 2 μm is less than about 10,000 particles/mL; and the concentration of particles with average particle size of less than about 10 μm is less than about 500 particles/mL.

In some of any embodiments, the formulation containing the TACI-Fc fusion protein is a low viscosity liquid that can flow.

In some of any embodiments, the viscosity of the TACI-Fc formulation is assessed at about 2° C. to about 8° C., optionally at or about 5° C. In some of any embodiments, the viscosity of the TACI-Fc formulation is assessed at about 12° C. to about 18° C., optionally at or about 15° C. In some of any embodiments, viscosity of the TACI-Fc formulation is assessed at about 18° C. to about 22° C., optionally at or about 20° C. In some of any embodiments, the viscosity of the TACI-Fc formulation is assessed at about 22° C. to about 28° C., optionally at or about 25° C.

In some of any embodiments, the viscosity of the TACI-Fc formulation is assessed at about 28° C. to about 32° C., such as at or about 30° C. In some of any embodiments, the TACI-Fc formulation has a viscosity of at or from about 3 cP to about 15 cP. In some of any embodiments, the TACI-Fc formulation has a viscosity of at or about 15 cP at 5° C. In some of any embodiments, the TACI-Fc formulation has a viscosity of at about 5 cP to about 10 cP, optionally about 6 cP to about 8 cP. In some of any embodiments, the TACI-Fc formulation has a viscosity of a viscosity of at or about 10 cP at 15° C. In some of any embodiments, the TACI-Fc formulation has a viscosity of at or about 7 cP at 25° C. In some of any embodiments, the viscosity of the TACI-Fc formulation is assessed using a rheometer or a cone and plate viscometer.

In some of any embodiments, the formulation containing a TACI-Fc fusion protein contains less than 6,000 ≥10 μm particles/mL. In some of any embodiments, the formulation containing a TACI-Fc fusion protein contains less than about 600 ≥25 μm particles/mL. In some of any embodiments, subvisible particles in the TACI-Fc formulation are assessed by Background Membrane Imaging (BMI) or a Liquid Particle Counter.

In some of any embodiments, the amount of the main species, high molecular weight species, and/or low molecular weight species of the TACI-Fc fusion protein in the formulation is assessed after storage. In some of any embodiments, the amount of the main species, high molecular weight species, and/or low molecular weight species of the TACI-Fc fusion protein in the formulation is assessed after storage at a storage temperature of about 2° C. to about 8° C. In some of any embodiments, the amount of the main species, high molecular weight species, and/or low molecular weight species of the TACI-Fc fusion protein in the formulation is assessed after storage at about 5° C. In some of any embodiments, the amount of the main species, high molecular weight species, and/or low molecular weight species of the TACI-Fc fusion protein in the formulation is assessed after storage at a storage temperature of about 12° C. to about 18° C. In some of any embodiments, the amount of the main species, high molecular weight species, and/or low molecular weight species of the TACI-Fc fusion protein in the formulation is assessed after storage at a storage temperature of about 15° C. In some of any embodiments, the amount of the main species, high molecular weight species, and/or low molecular weight species of the TACI-Fc fusion protein in the formulation is assessed after storage at a storage temperature of about 18° C. to about 22° C. In some of any embodiments, the amount of the main species, high molecular weight species, and/or low molecular weight species of the TACI-Fc fusion protein in the formulation is assessed after storage at a storage temperature of about 20° C. In some of any embodiments, the amount of the main species, high molecular weight species, and/or low molecular weight species of the TACI-Fc fusion protein in the formulation is assessed after storage at a storage temperature of about 22° C. to about 28° C. In some of any embodiments, the amount of the main species, high molecular weight species, and/or low molecular weight species of the TACI-Fc fusion protein in the formulation is assessed after storage at a storage temperature of about 25° C. In some of any embodiments, the amount of the main species, high molecular weight species, and/or low molecular weight species of the TACI-Fc fusion protein in the formulation is assessed after storage at for at least at or about 2 weeks. In some of any embodiments, the amount of the main species, high molecular weight species, and/or low molecular weight species of the TACI-Fc fusion protein in the formulation is assessed after storage at for at least at or about 1 year. In some of any embodiments, the amount of the main species, high molecular weight species, and/or low molecular weight species of the TACI-Fc fusion protein in the formulation is assessed after storage at for at least at or about 2 years.

In some of any embodiments, after storage for 12 months in liquid form at a storage temperature of at or about 2° C. to about 8° C., optionally at or about 5° C., and as measured by size exclusion chromatography (SEC) and as expressed in terms of area % under a curve that includes the TACI-Fc fusion protein in a form of a homodimer comprising two identical copies of the TACI-Fe fusion protein linked by a covalent disulfide bond as a main species, wherein:
the main species is at least 95%, at least 98%, or at least 99% of all observed species under the curve;
less than 5%, less than 2%, or less than 1% of species under the curve have a size greater than a size of the main species;
less than 5%, less than 2%, or less than 1% of species under the curve have a size less than a size of the main species;
the loss of the main species is less than 1%;
the increase in species under the curve having a size greater than a size of the main species is less than 1%; and/or
the increase in species under the curve having a size less than a size of the main species is less than 1%.

In some of any embodiments, the TACI-Fc fusion protein comprises a variant TACI polypeptide, and/or the TACI-Fc fusion protein has an isoelectric point (pI) of at or about 7.0 to about 7.8, at or about 7.0 to about 7.7, at or about 7.0 to about 7.6, at or about 7.0 to about 7.5, at or about 7.0 to about 7.3, at or about 7.1 to about 7.5, at or about 7.1 to about 7.3, or about 7.2.

In some of any embodiments, the formulation has a tonicity of an isotonic tonicity, of at or about 250 to about 350 mOsm/Kg, or of at or about 250 mOsm/Kg to about 600 mOsm/Kg.

In some of any embodiments, the formulation containing the TACI-Fc fusion protein is formulated for direct administration without dilution. In some of any embodiments, the formulation containing the TACI-Fc fusion protein is formulated for intravenous administration. In some of any embodiments, the formulation containing the TACI-Fc fusion protein is formulated for subcutaneous administration. In some of any embodiments, the formulation is a liquid formulation that has not been lyophilized and is not lyophilized prior to administration to a subject. In some of any embodiments, the formulation is a liquid formulation having a viscosity of at or about 3 cP to about 15 cP at 25° C. as assessed using a rheometer or a cone and plate viscometer. In some of any embodiments, the formulation is a liquid formulation comprising less than about 6,000 particles/mL having a particle size diameter of ≥10 μm; and/or comprising less than about 600 particles/mL having a particle size diameter of ≥25 μm, after storage for 12 months in liquid form at a storage temperature of at or about 2° C. to about 8° C., optionally at or about 5° C.

In some of any embodiments, the TACI-Fc fusion protein in the formulation contains (i) a TACI polypeptide sequence that is an extracellular domain (ECD) or portion of the ECD of a TACI polypeptide or is a variant TACI polypeptide thereof, and (ii) an Fc region.

In some of any embodiments, the TACI-Fc fusion protein in the formulation contains a variant TACI polypeptide containing one or more amino acid substitutions in the extracellular domain (ECD) of a reference TACI polypeptide, wherein the one or more amino acid substitutions comprise substitution(s) at positions selected from among 40, 59, 60, 61, 74, 75, 76, 77, 78, 79, 82, 83, 84, 85, 86, 87, 88, 92, 95, 97, 98, 99, 101, 102 and 103, corresponding to numbering of positions set forth in SEQ ID NO:122.

In some of any embodiments, the reference TACI polypeptide is a truncated polypeptide consisting of the extracellular domain of TACI or a specific binding portion thereof that binds to APRIL, BAFF, or a BAFF/APRIL heterotrimer.

In some of any embodiments, the reference TACI polypeptide comprises the sequence of amino acids set forth in SEQ ID NO:122, or a portion thereof containing one or both of a CRD1 domain and a CRD2 domain that binds to APRIL, BAFF, or a BAFF/APRIL heterotrimer.

In some of any embodiments, the reference TACI polypeptide lacks an N-terminal methionine.

In some of any embodiments, the reference TACI polypeptide comprises the CRD1 domain and the CRD2 domain.

In some of any embodiments, the reference TACI polypeptide the sequence set forth in SEQ ID NO:1.

In some of any embodiments, the reference TACI polypeptide consists of the sequence set forth in SEQ ID NO:1.

In some of any embodiments, the reference TACI polypeptide is a truncated wild-type TACI extracellular domain that contains the cysteine rich domain 2 (CRD2) but lacks the entirety of the cysteine rich domain 1 (CRD1), wherein the variant TACI polypeptide comprises one or more amino acid substitutions in the truncated wild-type TACI extracellular domain.

In some of any embodiments, the reference TACI polypeptide is a truncated wild-type TACI extracellular domain that consists of a contiguous sequence contained within amino acid residues 67-118 that includes amino acid residues 71-104, with reference to positions set forth in SEQ ID NO:122, wherein the variant TACI polypeptide comprises one or more amino acid substitutions in the truncated wild-type TACI extracellular domain.

In some of any embodiments, the reference TACI polypeptide is a truncated wild-type TACI extracellular domain that is 35, 36, 37, 38, 39, 40, 41, 42, 43, 44, 45, 46, 47, 48, 59, 50 or 51 amino acids in length.

In some of any embodiments, the reference TACI polypeptide consists essentially of the CRD2 domain.

In some of any embodiments, the reference TACI polypeptide is a truncated wild-type TACI extracellular domain that consists of amino acid residues 68-110 set forth in SEQ ID NO: 122.

In some of any embodiments, the reference TACI polypeptide comprises the sequence set forth in SEQ ID NO:13.

In some of any embodiments, the reference TACI polypeptide consists of the sequence set forth in SEQ ID NO:13.

In some of any embodiments, the reference TACI polypeptide the variant TACI polypeptide comprises 1, 2, 3, 4, 5, 6, 7, 8, 9, 10, 11, 12, 13, 14, or 15 amino acid substitutions.

In some of any embodiments, the TACI-Fc fusion protein in the formulation contains a variant TACI polypeptide that comprises two or more amino acid substitutions.

In some of any embodiments, the TACI-Fc fusion protein in the formulation contains a variant TACI polypeptide that comprises one or more amino acid substitutions are selected from W40R, Q59R, R60G, T61P E74V, Q75E, Q75R, G76S, K77E, F78Y, Y79F, L82H, L82P, L83S, R84G, R84L, R84Q, D85E, D85V, C86Y, I87L, I87M, S88N, I92V, Q95R, P97S, K98T, Q99E, A101D, Y102D, F103S, F103V, and F103Y, or a conservative amino acid substitution thereof. In some of any embodiments, the one or more amino acid substitutions comprises at least one of E74V, K77E, Y79F, L82H, L82P, R84G, R84L, R84Q, D85V, or C86Y.

In some of any embodiments, the one or more amino acid substitutions comprise an amino acid substitution selected from the group consisting of Q75E, K77E, F78Y, R84G, R84Q, A101D, and Y102D, or any combination thereof.

In some of any embodiments, the one or more amino acid substitutions comprise Q75E/R84Q, Q75E/K77E, Q75E/F78Y, Q75E/A101D, Q75E/Y102D, F77E/F78Y, K77E/R84Q, K77E/A101D, K77E/Y102D, F78Y/R84Q, F78Y/A101D, F78Y/Y102D, R84Q/A101D, R84Q/Y102D, or A101D/Y102D.

In some of any embodiments, the one or more amino acid substitutions are K77E/F78Y.

In some of any embodiments, the one or more amino acid substitutions are K77E/F78Y/Y102D.

In some of any embodiments, the variant TACI polypeptide has increased binding affinity to one or both of APRIL and BAFF compared to the reference TACI polypeptide.

In some of any embodiments, the variant TACI polypeptide has increased binding affinity to APRIL.

In some of any embodiments, the variant TACI polypeptide has increased binding affinity to BAFF.

In some of any embodiments, the variant TACI polypeptide has increased binding affinity to APRIL and BAFF. In some of any embodiments, the increased binding affinity for BAFF or APRIL is independently increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold or 60-fold.

In some of any embodiments, the variant TACI polypeptide has up to 10 amino acid modifications compared to the reference TACI polypeptide.

In some of any embodiments, the variant TACI polypeptide has up to 5 amino acid modifications compared to the reference TACI polypeptide.

In some of any embodiments, the variant TACI polypeptide has the at least 90% sequence identity to the amino acid sequence set forth in SEQ ID NO:122 or a specific binding fragment thereof containing the CRD1 domain and/or CRD2 domain. In some of any embodiments, the variant TACI polypeptide has at least 95% sequence identity to the amino acid sequence set forth in SEQ ID NO:122 or a specific binding fragment thereof containing the CRD1 domain and/or CRD2 domain.

In some of any embodiments, the specific binding fragment of the TACI polypeptide is set forth in SEQ ID NO:1, SEQ ID NO:13, SEQ ID NO:130, or SEQ ID NO:131.

In some of any embodiments, the variant TACI polypeptide has at least 90% sequence identity to the amino acid sequence set forth in SEQ ID NO:13. In some of any embodiments, the variant TACI polypeptide has at least 95% sequence identity to the amino acid sequence set forth in SEQ ID NO:13.

In some of any embodiments, the variant TACI polypeptide comprises the sequence of amino acids set forth in any one of SEQ ID NOS: 2-12, 21, 22 and 101-120; or the variant TACI polypeptide comprises the sequence of amino acids set forth in any one of SEQ ID NOS: 14-20, 23-35, 92-100 and 177-192.

In some of any embodiments, the variant TACI polypeptide consists or consists essentially of the sequence of amino acids set forth in any one of SEQ ID NOS: 2-12, 21, 22 and 101-120; or the variant TACI polypeptide consists or consists essentially of the sequence of amino acids set forth in any one of SEQ ID NOS: 14-20, 23-35, 92-100 and 177-192.

In some of any embodiments, the variant TACI polypeptide comprises the sequence of amino acids set forth in SEQ ID NO: 26.

In some of any embodiments, the sequence of the variant TACI polypeptide is set forth in SEQ ID NO: 26.

In some of any embodiments, the TACI is a TACI polypeptide that is a truncated wild-type TACI extracellular domain, wherein the truncated wild-type TACI extracellular domain contains the cysteine rich domain 2 (CRD2) but lacks the entirety of the cysteine rich domain 1 (CRD1).

In some of any embodiments, the TACI in the TACI-Fc fusion protein is a TACI polypeptide that is a truncated wild-type TACI extracellular domain, wherein the truncated wild-type TACI extracellular domain consists of a contiguous sequence contained within amino acid residues 67-118 that consists of amino acid residues 71-104, with reference to positions set forth in SEQ ID NO:122.

In some of any embodiments, the TACI in the TACI-Fc fusion protein is a TACI polypeptide that is a truncated wild-type TACI extracellular domain that is 35, 36, 37, 38, 39, 40, 41, 42, 43, 44, 45, 46, 47, 48, 59, 50 or 51 amino acids in length.

In some of any embodiments, the TACI in the TACI-Fc fusion protein is a TACI polypeptide that is a truncated wild-type TACI extracellular domain that consists of amino acid residues 68-110 set forth in SEQ ID NO: 122.

In some of any embodiments, the TACI in the TACI-Fc fusion protein is a TACI polypeptide that is a truncated wild-type TACI extracellular domain that consists of the sequence of amino acid set forth in SEQ ID NO:13.

In some of any embodiments, the TACI polypeptide in the TACI-Fc fusion protein is linked to an Fc region of an immunoglobulin via a linker, wherein the TACI-Fc fusion protein comprises the structure: TACI polypeptide (TACI)-Linker-Fc region.

In some of any embodiments, the TACI polypeptide in the TACI-Fc fusion protein is linked to an Fc region of an immunoglobulin via a linker that comprises a peptide linker and the peptide linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194), or combinations thereof.

In some of any embodiments, the Fc is an immunoglobulin Fc that is an IgG1 Fc domain, or is a variant Fc that exhibits reduced binding affinity to an Fc receptor and/or reduced effector function, optionally as compared to a wild-type IgG1 Fc domain.

In some of any embodiments, the Fc is an immunoglobulin Fc that is an IgG1 Fc domain and the Fc comprises the amino acid sequence set forth in SEQ ID NO:81.

In some of any embodiments, the Fc is an immunoglobulin Fc that is a variant IgG1 Fc domain containing one or more amino acid substitutions selected from L234A, L234V, L235A, L235E, G237A, S267K, R292C, N297G, and V302C, by EU numbering. In some of any embodiments, the immunoglobulin Fc region comprises the amino acid substitutions L234A, L235E and G237A by EU numbering. In some of any embodiments, the immunoglobulin Fc region is set forth in any of SEQ ID NOS: 73, 75, 83, 136, or 221. In some of any embodiments, the immunoglobulin Fc region further comprises the amino acid substitutions A330S and P331S. In some of any embodiments, the immunoglobulin Fc region is set forth in SEQ ID NO: 175 or SEQ ID NO:176.

In some of any embodiments, the Fe is a variant Fc containing the amino acid sequence set forth in SEQ ID NO:73.

In some of any embodiments, the TACI-Fc fusion protein is set forth in SEQ ID NO: 168.

In some of any embodiments, the TACI-Fc fusion protein is set forth in SEQ ID NO:170.

In some of any embodiments, the TACI-Fc fusion protein is set forth in SEQ ID NO: 167.

In some of any embodiments, the TACI-Fc fusion protein is set forth in SEQ ID NO:169.

In some of any embodiments, the TACI-Fc fusion protein is a homodimer containing two identical copies of the TACI-Fc fusion protein.

Also provided herein is a formulation containing a TACI-Fc fusion protein, containing: 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:167 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:167 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:167 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:167 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:167 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:167 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:168 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:168 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:168 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:168 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:168 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:168 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:169 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:169 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:169 linked by a covalent disulfide bond.

Also provided herein is a formulation containing a TACI-Fc fusion protein, containing 3.1% (w/v) arginine hydrochloride (Arg-HCl); 10 mM sodium acetate; and 0.02% (w/v) polysorbate 80 (PS80); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:169 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:169 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO:169 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:170 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.1% (w/v) poloxamer 188 (P188); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO: 170 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO:170 linked by a covalent disulfide bond.

Also provided herein is a formulation containing a TACI-Fc fusion protein, containing 3.1% (w/v) arginine hydrochloride (Arg-HCl) or at or about 135 mM to about 165 mM arginine or at or about 140 mM to about 150 mM arginine; 10 mM sodium acetate; and 0.02% (w/v) polysorbate 80 (PS80); wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO: 170 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 160 mg/mL; the formulation pH is at or about 4.7; and the TACI-Fc fusion protein is a homodimer containing two identical copies of the TACI-Fc fusion protein set forth in SEQ ID NO: 170 linked by a covalent disulfide bond.

Also provided herein is a formulation containing a TACI-Fc fusion protein, containing 2.0% or 3.0% (w/v) proline; 10 mM sodium acetate; 0.1% (w/v) poloxamer 188; and 50 mM sodium chloride; wherein the concentration of the TACI-Fc fusion protein in the composition is about 175 mg/mL; the formulation pH is at or about 4.7; and the TACI-Fc fusion protein is a homodimer containing two identical copies of the TACI-Fc fusion protein set forth in SEQ ID NO: 170 linked by a covalent disulfide bond.

In some of any embodiments, the TACI-Fc fusion protein neutralizes APRIL and BAFF. In some of any embodiments, the TACI-Fc fusion protein neutralizes APRIL and BAFF where the IC50 of the TACI-Fc fusion protein for neutralizing APRIL is less than 100 pM, less than 50 pM, less than 40 pM, less than 30 pM, less than 20 pM, less than 10 pM, less than 5 pM or less than 1 pM, or is any value between any of the foregoing; and/or the IC50 of the TACI-Fc for neutralizing BAFF is less than 400 pM, less than 300 pM, less than 200 pM, less than 100 pM, less than 75 pM, less than 50 pM, less than 25 pm, or less than 10 pM, or is any value between any of the foregoing.

In some of any embodiments, the TACI-Fc fusion protein blocks binding of APRIL, BAFF, or an APRIL/BAFF heterotrimer to BCMA or TACI; and/or the TACI-Fc fusion protein reduces the levels of circulating APRIL, BAFF, or an APRIL/BAFF in the blood following administration to a subject.

In some of any embodiments, the TACI-Fc fusion protein reduces or inhibits B cell maturation, differentiation and/or proliferation.

In some of any embodiments, the formulation containing the TACI-Fc fusion protein further contains an additional pharmaceutically acceptable excipient.

Also provided herein is an article of manufacture containing a TACI-Fc formulation provided herein in a vial or container. In some of any embodiments, the container is a syringe. In some of any embodiments, the container is an autoinjector.

Also provided herein is an article of manufacture containing the TACI-Fc formulation provided herein in a prefilled syringe.

Also provided herein is a kit containing a TACI-Fc formulation provided herein or an article of manufacture containing a TACI-Fc formulation provided herein, and instructions for use.

Also provided herein is a method of manufacturing or producing a TACI-Fc formulation provided herein.

Provided herein are methods of reducing an immune response in a subject, containing administering the formulation of any of the embodiments described herein to a subject in need thereof. In some of any embodiments, a B cell immune response is reduced in the subject, whereby B cell maturation, differentiation and/or proliferation is reduced or inhibited. In some of any embodiments, circulating levels of APRIL, BAFF or an APRIL/BAFF heterotrimer are reduced in the subject.

Also provided herein is a method of reducing circulating levels of APRIL, BAFF or an APRIL/BAFF heterotrimer in a subject containing administering the formulation of any of the embodiments described herein.

In some of any embodiments, reducing the immune response treats a disease, disorder or condition in the subject.

Also provided herein is a method of treating a disease, disorder or condition in a subject, containing administering a formulation the formulation of any of the embodiments described herein to a subject in need thereof.

In some of any embodiments, the disease, disorder or condition is an autoimmune disease or an inflammatory condition.

In some of any embodiments, the disease, disorder or condition is a B cell cancer, an antibody-mediated pathology, a renal disease, a graft rejection, graft versus host disease, or a viral infection.

In some of any embodiments, the disease, disorder or condition is selected from the group consisting of systemic lupus erythematosus (SLE), lupus nephritis, cutaneous lupus erythematosus, Sjögren's syndrome, scleroderma (systemic sclerosis), multiple sclerosis, diabetes (e.g. Type I diabetes), polymyositis, primary biliary cirrhosis, IgG4-related disease, IgA nephropathy, IgA vasculitis, ANCA vasculitis (microscopic polyangiitis, granulomatosis with polyangiitis [Wegener's granulomatosis], eosinophilic granulomatosis with polyangiitis [Churg-Strauss]) cryoglobulinemia, cold agglutinin or warm agglutinin disease, immune thrombocytopenic purpura, optic neuritis, amyloidosis, antiphospholipid antibody syndrome (APS), autoimmune polyglandular syndrome type II (APS II), autoimmune thyroid disease (AITD), Graves' disease, autoimmune adrenalitis, pemphigus vulgaris, bullous pemphigoid, myasthenia gravis, graft versus host disease (GVHD), transplantation, rheumatoid arthritis, acute lupus nephritis, amyotrophic lateral sclerosis, neuromyelitis optica, transverse myelitis, Rasmussen's encephalitis, CNS autoimmunity, Guillain-Barre syndrome, chronic inflammatory demyelinating polyneuropathy, neurocystercercosis, sarcoidosis, antiphospholipid antibody syndrome, IgG4-related disease, Hashimoto's thyroiditis, immune thrombocytopenia, Addison's Disease, dermatomyositis.

In some of any embodiments, the disease or disorder is autoantibody-associated glomerular disease.

In some of any embodiments, wherein the autoantibody-associated glomerular disease is immunoglobulin (Ig) A nephropathy (IgAN), lupus nephritis (LN), primary membranous nephropathy (pMN), myasthenia gravis, or renal anti-neutrophil cytoplasmic antibody (ANCA)-associated vasculitis (AAV).

In some of any embodiments, the disease, disorder or condition is immune thrombocytopenia, autoimmune hemolytic anemia, or cold agglutinin disease.

In some of any embodiments, the disease, disorder or condition immunoglobulin A (IgA) nephropathy.

In some of any embodiments, the disease, disorder or condition is a B cell cancer and the cancer is myeloma.

Also provided herein is a formulation of any of the embodiments described herein for use in reducing an immune response in a subject.

Also provided herein is use of formulation of any of the embodiments described herein in the manufacture of a medicament for reducing an immune response in a subject.

In some of any embodiments of the formulation for use or the uses provided herein, the immune response is a B cell immune response, wherein reducing the immune response reduces or inhibits B cell maturation, differentiation and/or proliferation. In some of any embodiments of the formulation for use or the uses provided herein, reducing the immune response reduces circulating levels of APRIL, BAFF or an APRIL/BAFF heterotrimer in the subject. In some of any embodiments of the formulation for use or the uses provided herein, reducing the immune response treats a disease, disorder or condition in the subject.

Also provided herein is a formulation of any of the embodiments described herein for use in treating a disease, disorder or condition in a subject.

Also provided herein is use of formulation of any of the embodiments described herein in the manufacture of a medicament for treating a disease, disorder or condition in a subject.

In some of any embodiments of the formulation for use or the uses provided herein, the disease, disorder or condition is an autoimmune disease or an inflammatory condition.

In some of any embodiments of the formulation for use or the uses provided herein, disease, disorder or condition is a B cell cancer, an antibody-mediated pathology, a renal disease, a graft rejection, graft versus host disease, or a viral infection.

In some of any embodiments of the formulation for use or the uses provided herein, disease, disorder or condition is selected from the group consisting of systemic lupus erythematosus (SLE), lupus nephritis, cutaneous lupus erythematosus, Sjögren's syndrome, scleroderma (systemic sclerosis), multiple sclerosis, diabetes (e.g. Type I diabetes), polymyositis, primary biliary cirrhosis, IgG4-related disease, IgA nephropathy, IgA vasculitis, ANCA vasculitis (microscopic polyangiitis, granulomatosis with polyangiitis [Wegener's granulomatosis], eosinophilic granulomatosis with polyangiitis [Churg-Strauss]) cryoglobulinemia, cold agglutinin or warm agglutinin disease, immune thrombocytopenic purpura, optic neuritis, amyloidosis, antiphospholipid antibody syndrome (APS), autoimmune polyglandular syndrome type II (APS II), autoimmune thyroid disease (AITD), Graves' disease, autoimmune adrenalitis, pemphigus vulgaris, bullous pemphigoid, myasthenia gravis, graft versus host disease (GVHD), transplantation, rheumatoid arthritis, acute lupus nephritis, amyotrophic lateral sclerosis, neuromyelitis optica, transverse myelitis, Rasmussen's encephalitis, CNS autoimmunity, Guillain-Barre syndrome, chronic inflammatory demyelinating polyneuropathy, neurocystercercosis, sarcoidosis, antiphospholipid antibody syndrome, IgG4-related disease, Hashimoto's thyroiditis, immune thrombocytopenia, Addison's Disease, dermatomyositis.

In some of any embodiments of the formulation for use or the uses provided herein, disease or disorder is autoantibody-associated glomerular disease.

In some of any embodiments of the formulation for use or the uses provided herein, autoantibody-associated glomerular disease is immunoglobulin (Ig) A nephropathy (IgAN), lupus nephritis (LN), primary membranous nephropathy (pMN), myasthenia gravis, or renal anti-neutrophil cytoplasmic antibody (ANCA)-associated vasculitis (AAV).

In some of any embodiments of the formulation for use or the uses provided herein, disease, disorder or condition is immune thrombocytopenia, autoimmune hemolytic anemia, or cold agglutinin disease.

In some of any embodiments of the formulation for use or the uses provided herein, disease, disorder or condition is a B cell cancer, optionally wherein the cancer is myeloma.

Also provided herein is a method for reducing the loss of a TACI-Fc fusion protein homodimer in a liquid pharmaceutical formulation containing a TACI-Fc fusion protein at a concentration of at least 160 mg/mL, containing the step of combining the TACI-Fc fusion protein with a viscosity-reducing concentration of a tonicifying agent comprising arginine.

### BRIEF DESCRIPTION OF THE DRAWINGS

FIG. 1 depicts the viscosity measurements for formulations 1-20 at 160 mg/ml time=0.

FIG. 2 depicts the viscosity measurements for formulations #1-4 at time=0, 8 weeks at 4° C., and 8 weeks at 30° C.

FIGS. 3A-3C depict the results of a long-term stability study for formulations 1-20 at 2-8° C. as measured by HP-SEC. FIG. 3A depicts the rate of change per week of % high molecular weight (HMW) species. FIG. 3B depicts the rate of change per week of the % main species. FIG. 3C depicts the rate of change per week of the % low molecular weight (LMW) species.

FIGS. 4A-4E depict the results of a long-term stability study for formulations 1-20 at 30° C. as measured by HP-SEC. FIG. 4A depicts the rate of change per week of % high molecular weight (HMW) species. FIG. 4B depicts the rate of change per week of % main species. FIG. 4C depicts the rate of change per week of the % low molecular weight (LMW) species. FIG. 4D depicts a subset of the conditions from FIGS. 4A, while FIG. 4E depicts a subset of the conditions from FIG. 4B.

FIGS. 5A-5C depict particle counts by background membrane imaging (BMI) measured at the final timepoint of 8 weeks of storage for formulations 1-20 at 2-8° C. FIG. 5A depicts particles greater than or equal to 2 μm. FIG. 5B depicts particles greater than or equal to 10 μm. FIG. 5C depicts particles greater than or equal to 25 μm.

FIGS. 6A-6C depict particle counts by background membrane imaging (BMI) measured at the final timepoint of 8 weeks of storage for formulations 1-20 at 30° C. FIG. 6A depicts particles greater than or equal to 2 μm. FIG. 6B depicts particles greater than or equal to 10 μm. FIG. 6C depicts particles greater than or equal to 25 μm.

FIG. 7 depicts the viscosity measurements of formulations 21-40 with ranges from approximately 7.3-5.2 cP at time=0.

FIGS. 8A-8C depict the results of a long-term stability study for formulations 21-40 at 2-8° C. as measured by HP-SEC. FIG. 8A depicts the rate of change per week of % total high molecular weight (HMW) species. FIG. 8B depicts the rate of change per week of % main species. FIG. 8C depicts the rate of change per week of % low molecular weight (LMW) species.

FIGS. 9A-9E depict the results of a long-term stability study for formulations 21-40 at 30° C. as measured by HP-SEC. FIG. 9A depicts the rate of change per week of % high molecular weight (HMW) species. FIG. 9B depicts the rate of change per week of % main species. FIG. 9C depicts the rate of change per week of % low molecular weight (LMW) species. FIG. 9D depicts a subset of the conditions from FIGS. 9A, while FIG. 9E depicts a subset of the conditions from FIG. 9B.

FIGS. 10A-10C depict particle counts by background membrane imaging (BMI) measured at the final timepoint after 8 weeks of storage for formulations 21-40 at 2-8° C. FIG. 10A depicts the number of particles greater than or equal to 2 μm. FIG. 10B depicts number of particles greater than or equal to 10 μm. FIG. 10C depicts number of particles greater than or equal to 25 μm.

FIGS. 11A-11C depict particle counts by background membrane imaging (BMI) measured at 2, 4, and 8 weeks of storage for formulations 21-40 at 30° C. FIG. 11A depicts particles greater than or equal to 2 μm. FIG. 11B depicts particles greater than or equal to 10 μm. FIG. 11C depicts particles greater than or equal to 25 μm.

FIGS. 12A-12C depict particle counts by background membrane imaging (BMI) measured at the final timepoint of 8 weeks of storage for formulations 21-40 at 30° C. FIG. 12A depicts particles greater than or equal to 2 μm. FIG. 12B depicts particles greater than or equal to 10 μm. FIG. 12C depicts particles greater than or equal to 25 μm.

FIG. 13 depicts the comparison of viscosity curves for formulations 41-46.

FIGS. 14A-14I: FIGS. 14A-14F depict the results from size exclusion chromatography (SEC) and reduced capillary electrophoresis sodium dodecyl sulfate (rCE-SDS) assays to assess stability. FIG. 14A depicts the rate of change in % main species by SEC at−70° C., −20° C., and 5° C. FIG. 14B depicts the rate of change in % main species by SEC at 25° C. and 40° C. FIG. 14C depicts the % main species by SEC during storage at 5° C. for 24 months. FIG. 14D depicts % main rate by rCE-SDS at −70° C., −20° C., and 5° C. FIG. 14E depicts % main rate by rCE-SDS at 25° C. and 40° C. 14F depicts % main rate by rCE-SDS after storage at 5° C. for 24 months. FIGS. 14G-14I depict the result from an imaged capillary isoelectric focusing (icIEF) assay. FIG. 14G depicts the % main species by icIEF during storage at 5° C. for 24 months. FIG. 14H depicts the % acidic species by icIEF during storage at 5° C. for 24 months. FIG. 14I depicts % basic species by icIEF during storage at 5° C. for 24 months.

FIGS. 15A-D depict particle counts by light obscuration. FIG. 15A and FIG. 15B depict counts for particles greater than or equal to 2 μm at 5° C. and 25° C. respectively. FIG. 15C and FIG. 15D depict counts for particles greater than or equal to 10 μm at 5° C. and 25° C. respectively.

FIGS. 16A-F depict the potency by ELISA of TACI-Fc protein in different formulation buffers over a 104-week period. FIG. 16A and FIG. 16C depict potency in a 0.015% PS80 w/v formulation for BAFF and APRIL respectively. FIG. 16B and FIG. 16D depict potency in a 0.1% P188 w/v formulation for BAFF and APRIL respectively. FIG. 16E and FIG. 16F depict potency for formulations in PS80 and P188 after storage at 5° C. for APRIL and BAFF, respectively.

FIG. 17 depicts recorded temperatures in Celsius (° C.) during transport of formulation buffers 48-62.

FIGS. 18A-18C depicts results from a size exclusion chromatography (SEC) assay after a simulated transport study at varying poloxamer 188 concentrations for formulations 48-62. In addition to the two replicates transported, there is a control condition where samples were stored quiescent at 2-8° C. and not subjected to transport. FIG. 18A depicts 175 mg/mL TACI-Fc protein in 10 mM sodium acetate, 3.1% arginine HCl (w/v) at pH 4.6. FIG. 18B depicts 175 mg/mL TACI-Fc protein in 10 mM sodium acetate, 2% proline (w/v), 50 mM NaCl at pH 4.6. FIG. 18C depicts 160 mg/mL TACI-Fc protein in 10 mM sodium acetate, 2% proline (w/v), 50 mM NaCl at pH 4.6.

FIGS. 19A-19F depict particle counts by light obscuration after simulated transport compared to controls that were not subject to simulated transport at varying poloxamer 188 concentrations. FIG. 19A and FIG. 19B depict particle counts in formulation buffer 175 mg/mL TACI-Fc protein in 10 mM sodium acetate, 3.1% arginine HCl (w/v) at pH 4.6 for particles greater than or equal to 2 μm and 10 μm, respectively. FIG. 19C and FIG. 19D depict particle counts in formulation buffer 175 mg/mL TACI-Fc protein in 10 mM sodium acetate, 2% proline (w/v), 50 mM NaCl at pH 4.6 for particles greater than or equal to 2 μm and 10 μm, respectively. FIG. 19E and FIG. 19F depict particle counts in formulation buffer 160 mg/mL TACI-Fc protein in 10 mM sodium acetate, 2% proline (w/v), 50 mM NaCl at pH 4.6 for particles greater than or equal to 2 μm and 10 μm, respectively.

FIGS. 20A-20F depict potency by ELISA of BAFF and APRIL in various formulation buffers for a simulated transport condition at varying poloxamer 188 concentrations compared to a control. FIG. 20A and FIG. 20B depict potency of 175 mg/mL TACI-Fc protein in 10 mM sodium acetate, 3.1% arginine HCl (w/v) at pH 4.6 for BAFF and APRIL, respectively. FIG. 20C and FIG. 20D depict potency of 175 mg/mL TACI-Fc protein in 10 mM sodium acetate, 2% proline (w/v), 50 mM NaCl at pH 4.6 for BAFF and APRIL, respectively. FIG. 20E and FIG. 20F depict potency of 160 mg/mL TACI-Fc protein in 10 mM sodium acetate, 2% proline (w/v), 50 mM NaCl at pH 4.6 for BAFF and APRIL respectively.

FIGS. 21A-21D depict results from a size exclusion chromatography (SEC) assay on formulations 51, 56, and 61. FIG. 21A depicts change in percent main per week at −70° C., −20° C., and 5° C. FIG. 21B depicts change in percent main per week at 25° C. and 40° C. FIG. 21C depicts change in percent total high molecular weight (HMW) per week at −70° C., −20° C., and 5° C. FIG. 21D depicts change in percent total HMW per week at 25° C. and 40° C.

FIGS. 22A-22C depict assessments of percent main (FIG. 22A), high molecular weight (HMW) (FIG. 22B), and low molecular weight (LMW) (FIG. 22C) for formulations 51, 56, and 61 over time at 5° C.

FIGS. 23A and 23B depict results from a reduced capillary electrophoresis sodium dodecyl sulfate (rCE-SDS) on formulations 51, 56, and 61 to assess rate of change by percent total purity per week. FIG. 23A depicts results from incubation temperatures of −70° C., −20° C., and 5° C. FIG. 23B depicts results from incubation temperatures of 25° C. and 40° C.

FIGS. 24A and 24B depict results from a reduced capillary electrophoresis sodium dodecyl sulfate (rCE-SDS) on formulations 51, 56, and 61 to assess percent total purity during storage. FIG. 24A depicts results at 5° C. incubation and FIG. 24B depicts results after 25° C. incubation.

FIGS. 25A and 25B depict results from an imaged capillary isoelectric focusing (icIEF) assay to assess rate of change as percent main per week for formulation 51, 56, and 61. FIG. 25A depicts results from incubation temperatures of −70° C., −20° C., and 5° C. FIG. 25B depicts results from incubation temperatures of 25° C. and 40° C.

FIGS. 26A-C depict results from an imaged capillary isoelectric focusing (icIEF) assay to assess percent main, percent acidic, and percent basic for formulation 51, 56, and 61 after 5° C. incubation. FIG. 26A depicts results for percent main species, FIG. 26B depicts results for percent acidic species, and FIG. 26C depicts results for percent basic species.

FIGS. 27A-D depict results from an assessment of subvisible particles by light obscuration formulated in formulations 51, 56, and 61 during long term storage. FIGS. 27A and 27C depict particle counts out to 52 weeks at 5° C. for particles greater than or equal to 2 μm or 10 μm, respectively. FIGS. 27B and 27D depict particle counts out to 26 weeks at 25° C. for particles greater than or equal to 2 μm and 10 μm, respectively.

FIGS. 28A-28C depict results from a bioactivity assessment by ELISA (percent relative potency of BAFF) conducted on formulation buffers 51, 56, and 61 at −70° C., −20° C., 2-8° C., 25° C., and 40° C. over time. FIG. 28A depicts formulation 51, FIG. 28B depicts formulation 56, and FIG. 28C depicts formulations 61.

FIGS. 29A-29C depict results from a bioactivity assessment by ELISA (percent relative potency of APRIL) conducted on formulation buffers 51, 56, and 61 at −70° C., −20° C., 2-8° C., 25° C., and 40° C. over time. FIG. 29A depicts formulation 51, FIG. 29B depicts formulation 56, and FIG. 29C depicts formulations 61.

FIGS. 30A-C depict assessments of change in percent main per week of various formulation buffers at one of four pHs (pH 4.3, 4.4, 4.6, and 5.0) by size exclusion chromatography (SEC). FIG. 30A depicts results for formulations after 12 weeks at 5° C., FIG. 30B depicts results for formulations after 12 weeks at 25° C., and FIG. 30C depicts results from formulations after 12 weeks at 40° C.

FIGS. 31A-C depicts results from a reduced capillary electrophoresis sodium dodecyl sulfate (rCE-SDS) to assess change in percent main/week for various buffers. FIG. 31A depicts results after 12 weeks at 5° C., FIG. 31B depicts results after 12 weeks at 25° C., and FIG. 31C depicts results after 12 weeks at 40° C.

FIGS. 32A-C depicts results from an imaged capillary isoelectric focusing (icIEF) assay to assess change in percent main/week for various buffers. FIG. 32A depicts results after 12 weeks at 5° C., FIG. 32B depicts results after 12 weeks at 25° C., and FIG. 32C depicts results after 12 weeks at 40° C.

FIGS. 33A-F depicts results from an imaged capillary isoelectric focusing (icIEF) assay to assess the change in basic and acidic species of the TACI-Fc by change in percent species per week. FIGS. 33A-C depict the percent basic per week after 12 weeks at 5° C., 25° C., and 40° C., respectively. FIGS. 33D-F depict the percent acid per week after 12 weeks at 5° C., 25° C., and 40° C., respectively.

FIGS. 34A-I depict particle counts by flow imaging for formulation 65 and 66 at 0, 0.5, 1, 2, and 3 months of storage. FIGS. 34A, 34B, and 34C depict counts for particles greater than or equal to 2 μm at 5° C., 25° C., and 40° C., respectively. FIGS. 34D, 34E, and 34F depict counts for particles greater than or equal to 10 μm at 5° C., 25° C., and 40° C., respectively. FIGS. 34G, 34H, and 34I depict counts for particles greater than or equal to 25 μm at 5° C., 25° C., and 40° C., respectively.

FIGS. 35A-H depict the potency by ELISA and cell-based assay (CBA) of TACI-Fc for BAFF and APRIL in different formulations. FIGS. 35A and 35B depict percent relative potency for Formulation 66 at −70° C., 4° C., 25° C., and 40° C. for ELISA APRIL and ELISA BAFF, respectively. FIGS. 35C and 35D depict percent relative potency for Formulation 67 at 4° C., 25° C., and 40° C. for ELISA APRIL and ELISA BAFF, respectively. FIGS. 35E and 35G depict percent relative potency for Formulation 66 at −70° C., 4° C., 25° C., and 40° C. for CBA APRIL and CBA BAFF, respectively. FIGS. 35F and 35H depict percent relative potency for Formulation 67 at 4° C., 25° C., and 40° C. for CBA APRIL and CBA BAFF, respectively.

### DETAILED DESCRIPTION

Provided herein are formulations comprising a TACI-Fc fusion protein that are stable. The TACI-Fc formulations provided herein contain components that permit formulation at high concentrations of TACI-Fc protein in the formulation where the TACI-Fc maintains stability after storage. The TACI-Fc formulation provided herein can be dispensed precisely and is stable with respect to TACI-Fc decomposition products and aggregates over the duration of storage, including storage at refrigerated and increased temperatures, and after transport. As described herein, formulations provided herein are highly stable upon storage, such as prolonged storage at altered temperature, maintaining substantially the same viscosity, and/or showing little or no protein aggregation upon storage. In some embodiments, at least about 95% of the TACI-Fc protein in the formulation is in the homodimer form (or “main species” or “main” form) as measured by size exclusion chromatography (SEC) before and/or after storage. In some embodiments, the formulation comprises no more than about 5% of formation of low and high molecular weight species as measured by size exclusion chromatography before and/or after storage. Moreover, the low viscosity of the presently described formulations in spite of high protein concentrations permits convenient processing, including but not limited to ultrafiltration and sterile filtration, and injection of the drug product solution at relatively low total volumes through a syringe needle, for example. These properties make the TACI-Fc formulation particularly suitable as a pharmaceutical product that can be administered to human patients, for example by subcutaneous administration.

The provided formulations comprise a TACI-Fc fusion protein, such as variant TACI-fusion proteins, that bind to BAFF or APRIL ligands to neutralize their activity and block or antagonize the activity of B cell stimulatory receptors. Among the provided formulations are those comprising variant TACI polypeptides, such as those described herein and in International Patent Publication Nos. WO2021226551 and WO 2022236335. In embodiments herein, the provided formulations comprise immunomodulatory proteins that are fusion proteins of a TACI extracellular domain or binding portion thereof (hereinafter TACI ECD) and a multimerization domain that is immunoglobulin Fc (e.g., a TACI-Fc fusion protein). In embodiments, the formulations provided herein comprise a TACI-Fc fusion protein comprising a variant TACI polypeptide, such as those described herein and in International Patent Publication Nos. WO2021226551 and WO 2022236335.

In embodiments herein, the provided formulations contain a TACI-Fc fusion protein that has significantly improved ligand affinity; is superior to WT TACI-Ig, BAFF and/or APRIL-only inhibitors; and is well-tolerated in healthy adults via IV or SC administration with dose-dependent PK/PD. In embodiments, herein the provided formulations contain TACI-Fc fusion proteins and immunomodulatory proteins comprising the TACI-Fc fusion proteins that are suitable for multiple autoantibody-related inflammatory diseases.

In embodiments herein, formulations containing variant TACI polypeptides (e.g., variant TACI-Fc fusion proteins) such as those described herein and set forth in International Patent Publication Nos. WO2021226551 and WO 202223633, can be administered at a reduced dosing frequency compared to wild-type TACI. This reduced dose frequency may provide a treated subject with better symptom control, improve adherence to the dosing regimen, increase patient quality of life or patient satisfaction and/or overall reduce the costs of receiving the treatment. Moreover, reducing the dose, even at a more regular frequency such as once weekly, may also mitigate against certain adverse effects, such as those associated with administration.

In some embodiments, the formulations containing the TACI-Fc fusion proteins provided herein can be used for the treatment of diseases, disorders or conditions that are associated with a dysregulated immune response, such as associated with inflammatory or autoimmune symptoms including an inflammatory disease or an autoimmune disease.

The formulations provided herein contain a TACI-Fc fusion protein at high concentrations of greater than 100 mg/mL. High concentrations of protein therapeutics can lead to protein instability that can lead to protein aggregation or degradation, which can decrease or eliminate the functionality of the protein (e.g., TACI-Fc fusion protein). Protein aggregation and/or degradation also can lead to undesirable side effects upon administration, such as immune reactions at the injection site. Aggregation of the protein in a pharmaceutical formulation also can decrease the amount of the protein in the active state. For example, aggregation of a TACI-Fc fusion protein into high molecular weight (HMW) species (i.e., greater than two TACI-Fc polypeptide chains e.g., greater than about 62.6 kDa) can titrate away the TACI-Fc polypeptide(s) from the active homodimer form (i.e., the “main species”). Protein aggregation and/or degradation in a pharmaceutical formulation containing a highly concentrated protein therapeutic also can result in viscous formulations that are difficult to administer. Highly viscous pharmaceutical formulations can have decreased syringeability and/or injectability. Provided herein are TACI-Fc formulations that contain a high concentration of TACI-Fc fusion protein and other excipients as described herein and maintain the majority of the TACI-Fc fusion protein in the main, homodimer state, and are more easily administered.

In embodiments herein the provided formulations contain TACI-Fc fusion proteins at a concentration of greater than 100 mg/mL, such as at or about 140 mg/mL to about 230 mg/mL, at or about 140 mg/mL to about 200 mg/mL, at or about 160 mg/mL, or at or about 175 mg/mL. Even at these high concentrations, the majority of the TACI-Fc is the main species (homodimeric form). Also, as shown herein, even at these high concentrations the majority of the TACI-Fc protein is in the main species (homodimeric form) after prolonged storage, such as prolonged storage at low (e.g., 2-8° C., or 5° C.) storage temperatures for up to about 1, 2, or 3 years or high (e.g., 30° C.) for up to about 6 months.

In embodiments herein the provided formulations contain TACI-Fc fusion proteins that are well tolerated for administration at low doses (e.g., 80 mg) to high doses (e.g., 960 mg) without adverse effects. In embodiments herein, the provided formulations contain TACI-Fc fusion proteins that are also well tolerated when administered once every four weeks (Q4W). Furthermore, in embodiments herein the provided formulations contain TACI-Fc fusion proteins that are effective whether injected SC or IV at low doses (e.g., 80 mg).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and the appended claims, the singular forms “a,” “an,” and “the” include plural referents unless the context clearly indicates otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about” X or “at or about” X includes description of “X”. In some embodiments, a numerical range or numerical value such as a concentration or pH range may be presented as: “at or about” value X to “about” value Y, for example (or similar language “at or about X to Y” or “at or about X to at or about Y”), or “between about” value X to “between about” value Y (or similar language “between about X to Y”), for example, or “at or about” value X. Such ranges herein in each case include each of the endpoint X and Y values, as well as the exact range from X to Y or the exact value X, as well as a range from “about X” to “about Y.”

The term “affinity-modified” as used in the context of a domain of a protein means a mammalian protein having an altered amino acid sequence in an extracellular domain or a specific binding portion thereof (relative to the corresponding wild-type parental or unmodified domain) such that it has an increased or decreased binding activity, such as binding affinity, to at least one of its binding partners (alternatively counter-structures) compared to the parental wild-type or unmodified (i.e., non-affinity modified domain) protein. In some embodiments, the affinity-modified domain can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions, in a wild-type or unmodified domain. An increase or decrease in binding activity, e.g. binding affinity, can be determined using well known binding assays, including flow cytometry. Larsen et al., American Journal of Transplantation, Vol 5: 443-453 (2005). See also, Linsley et al., Immunity, 1: 7930801 (1994). An increase in a protein's binding activity, e.g. affinity, to its binding partner(s) is to a value at least 10% greater than that of the wild-type control and in some embodiments, at least 20%, 30%, 40%, 50%, 100%, 200%, 300%, 500%, 1000%, 5000%, or 10000% greater than that of the wild-type control value. A decrease in a protein's binding activity, e.g. affinity, to at least one of its binding partners is to a value no greater than 90% of the control but no less than 10% of the wild-type control value, and in some embodiments no greater than 80%, 70% 60%, 50%, 40%, 30%, or 20% but no less than 10% of the wild-type control value. An affinity-modified protein is altered in primary amino acid sequence of the extracellular domain or a specific binding portion thereof by substitution, addition, or deletion of amino acid residues. The term affinity-modified is not to be construed as imposing any condition for any particular starting composition or method by which the affinity-modified protein was created. Thus, an affinity-modified protein is not limited to wild-type protein domains that are then transformed to an affinity-modified domain by any particular process of affinity modification. An affinity-modified domain polypeptide can, for example, be generated starting from wild-type mammalian domain sequence information, then modeled in silico for binding to its binding partner, and finally recombinantly or chemically synthesized to yield the affinity-modified domain composition of matter. In but one alternative example, an affinity-modified domain can be created by site-directed mutagenesis of a wild-type domain. Thus, “affinity modified TD domain” denotes a product and not necessarily a product produced by any given process. A variety of techniques including recombinant methods, chemical synthesis, or combinations thereof, may be employed.

The term “affinity-modified TD domain” refers to an affinity-modified domain of a member of the tumor necrosis factor receptor superfamily (TNFRSF) protein or a TNF ligand thereof having an altered amino acid sequence of a TNFR domain or of a TNF domain therein, respectively. For example, an affinity-modified TD domain of a TNFRSF protein has an altered amino acid sequence of a TNFR domain composed of at least one cysteine rich domain (CRD) within the extracellular domain of the TNFRSF protein or a specific binding portion thereof (relative to the corresponding wild-type parental or unmodified domain) such that it has an increased or decreased binding activity, such as binding affinity, to at least one of its binding partners (alternatively counter-structures) compared to the parental wild-type or unmodified protein containing the non-affinity modified or unmodified TD domain.

An “affinity-modified TACI” (also referred to as a variant TACI) refers to a TACI protein molecule that antagonizes or blocks the activity of a B cell stimulatory receptor. For example, TACI binds to APRIL and/or BAFF, which are ligands of the B cell stimulatory receptors B cell maturation antigen (BCMA), B cell activation factor receptor (BAFF-R), and transmembrane activator and calcium modulator and cyclophilin ligand interactor (TACI). In particular embodiments, a BIM includes the extracellular domain of TACI, or a portion of the extracellular domain of TACI containing a TNF receptor family domain (e.g. TD, e.g. CRD) that binds to cognate ligands APRIL and/or BAFF, and heterotrimers of APRIL and BAFF. An affinity-modified variant of the extracellular domain or portion thereof of TACI can include one more amino acid modifications (e.g. amino acid substitutions) in the TD that increase binding affinity for the cognate ligand (e.g. APRIL and/or BAFF, and heterotrimers of APRIL and BAFF).

As used herein, a “main species” of TACI-Fc protein is used interchangeably with “main” and refers to a disulfide-linked homodimer consisting of two identical TACI-Fc fusion polypeptide chains, each with a TACI domain-Fc fusion polypeptide, where the identical chains in the pair are linked by covalent disulfide bonds. In a formulation of purified TACI-Fc protein, the majority of TACI-Fc in the formulation is a homodimer of the two TACI-Fc fusion polypeptide chains. The molecular weight of the main TACI-Fc fusion protein is less than the molecular weight of a high molecular weight species of a TACI-Fc fusion protein. The molecular weight of the main TACI-Fc fusion protein is more than the molecular weight of a low molecular weight species of a TACI-Fc fusion protein. The main species of a TACI-Fc fusion protein, comprising two chains, each comprising the CRD2 of TACI (e.g., set forth in SEQ ID NO. 13 or containing amino acid modifications in the sequence of amino acids set forth in SEQ ID NO. 13) fused to an Fc domain, has a molecular weight of approximately 62.6 kDa. The molecular weight of the main species of a TACI-Fc fusion protein of SEQ ID NO. 167 is approximately 62.6 kDa. The molecular weight of the main species of the TACI-Fc fusion protein can depend on the length of the TACI polypeptide in the fusion protein. In embodiments herein, the molecular weight of the main species of a TACI-Fc fusion protein comprising the full ECD of TACI can be at or about 116.4 kDa. In embodiments herein, the molecular weight of the main species of a TACI-Fc protein is at or about 62.6 kDa to at or about 116.4 kDa. In some embodiments the TACI polypeptide in the TACI-Fc fusion protein in the main species does not contain the entire CRD2 and the molecular weight of the TACI-Fc main is less than about 62.6 kDa. In embodiments herein, the approximate molecular weight of the main TACI-Fc species is calculated by taking the molecular weight of the TACI-Fc fusion protein and multiplying by two.

As used herein, a “high molecular weight” (HMW) species of TACI-Fc protein refers a species of TACI-Fc protein that comprises more TACI-Fc polypeptide chains than a dimer of two identical TACI-Fc fusion polypeptide chains. The HMW species of a TACI-Fc fusion protein contains more TACI domain-Fc fusion polypeptide chains than the main species of a TACI-Fc fusion protein. In some examples, a HMW species of a TACI-Fc protein contains 3, 4, 5, 6, or more TACI-Fc fusion polypeptide chains. The molecular weight of the HMW species of a TACI-Fc fusion protein is higher than the molecular weight of the main species of a corresponding TACI-Fc fusion protein. In embodiments herein, the molecular weight of the HMW species of a TACI-Fc fusion protein comprising the full ECD of TACI is greater than about 116.4 kDa. The molecular weight of a HMW species of a TACI-Fc fusion protein of SEQ ID NO. 167 is greater than approximately 62.6 kDa. In embodiments herein, the molecular weight of the HMW species of a TACI-Fc protein is greater than 62.6 kDa. In embodiments herein, the molecular weight of the HMW species of a TACI-Fc protein is at least 93.9 kDa. In embodiments herein, the approximate molecular weight of the high molecular weight species of the TACI-Fc protein can be calculated by taking the molecular weight of the TACI-Fc fusion protein and multiplying by the number of TACI-Fc fusion proteins in the HMW species (e.g., >2).

As used herein, a “low molecular weight” (LMW) species of TACI-Fc protein refers a species of TACI-Fc protein that comprises fewer TACI-Fc polypeptide chains than a dimer of two identical TACI-Fc fusion polypeptide chains. The LMW species of a TACI-Fc fusion protein contains fewer TACI domain-Fc fusion polypeptide chains than the main species of a TACI-Fc fusion protein. In some examples, a LMW species of a TACI-Fc protein contains 1 or fewer TACI-Fc fusion polypeptide chains. The molecular weight of the LMW species of a TACI-Fc fusion protein is less than the molecular weight of the main species of a corresponding TACI-Fc fusion protein. The molecular weight of a LMW species of a TACI-Fc fusion protein of SEQ ID NO. 167 is less than approximately 62.6 kDa, such as at or about 31.3 kDa. In embodiments herein, the molecular weight of the LMW species of a TACI-Fc fusion protein comprising the full ECD of TACI is less than about 116.4 kDa, such as at or about 58.3 kDa. In embodiments herein, the molecular weight of the low molecular weight species of the TACI-Fc protein is calculated by taking the molecular weight of a single TACI-Fc fusion polypeptide chain and multiplying by the number of TACI-Fc fusion polypeptide chains in the LMW species (e.g., 1).

As used herein, a “B cell stimulatory receptor” refers to one or more of B cell maturation antigen (BCMA), B cell activation factor receptor (BAFF-R), and transmembrane activator and calcium modulatory and cyclophilin ligand interactor (TACI), which are related tumor necrosis factor (TNF) superfamily receptors expressed on B cells. Engagement or ligation of these related receptors by their cognate ligands, BAFF and/or APRIL, or heterotrimers of APRIL and BAFF, regulate B cell homeostasis, including B cell survival, B cell maturation and differentiation and immunoglobulin class switching. A B cell stimulatory receptor generally contains an extracellular portion, a transmembrane domain and cytoplasmic region, in which the cytoplasmic region contains one or more TNF receptor associated factor (TRAF) binding sites. Recruitment of various TRAF molecules to the cytoplasmic domain can activate various transcription factors, such as NF-κB (e.g. NF-κB1 or NF-κB2), to mediate B cell signaling pathways regulating B cell homeostasis.

As used herein, “bind,” “bound,” or grammatical variations thereof refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs.

As used herein, “binding activity” refers to characteristics of a molecule, e.g. a polypeptide, relating to whether or not, and how, it binds one or more binding partners. A binding activity can include any measure of binding of one molecule for a binding partner. Binding activities include the ability to bind the binding partner(s), the affinity with which it binds to the binding partner (e.g. high affinity), the avidity with which it binds to the binding partner, the strength of the bond with the binding partner and/or specificity or selectivity for binding with the binding partner.

The term “binding affinity” as used herein means the specific binding affinity of a protein for its binding partner (i.e., its counter-structure) under specific binding conditions. The binding affinity refers to the strength of the interaction between two or more molecules, such as binding partners, typically the strength of the noncovalent interactions between two binding partners. An increase or attenuation in binding affinity of an affinity-modified domain, or an immunomodulatory protein containing an affinity-modified domain, to a binding partner is determined relative to the binding affinity of the unmodified domain (e.g., the native or wild-type TD domain). Methods for determining binding affinity, or relative binding affinity, are known in art, and include solid-phase ELISA immunoassays, ForteBio® Octet™, Biacore® measurements or flow cytometry. See, for example, Larsen et al., American Journal of Transplantation, vol. 5: 443-453 (2005); Linsley et al., Immunity, Vol 1 (9): 793-801 (1994). In some embodiments, binding affinity can be measured by flow cytometry, such as based on a Mean Fluorescence Intensity (MFI) in a flow binding assay.

The term “binding avidity” as used herein means the specific binding avidity of a protein for its binding partner (i.e., its counter-structure) under specific binding conditions. In biochemical kinetics, “avidity” refers to the accumulated strength of multiple affinities of individual non-covalent binding interactions, such as between a protein for its binding partner (i.e., its counter-structure). As such, avidity is distinct from affinity, which describes the strength of a single interaction.

The term “biological half-life” refers to the amount of time it takes for a substance, such as an immunomodulatory protein, to lose half of its pharmacologic or physiologic activity or concentration. Biological half-life can be affected by elimination, excretion, degradation (e.g., enzymatic degradation/digestion) of the substance, or absorption and concentration in certain organs or tissues of the body. In some embodiments, biological half-life can be assessed by determining the time it takes for the blood plasma concentration of the substance to reach half its steady state level (plasma half-life). Conjugates that can be used to derivatize and increase the biological half-life of a protein are known in the art and include, but are not limited to, multimerization domains (e.g. Fc immunoglobulin domain), polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), polyglutamic acid (glutamylation).

The term “cell surface counter-structure” (alternatively “cell surface binding partner”) as used herein is a counter-structure (alternatively is a binding partner) expressed on a mammalian cell. Typically, the cell surface binding partner is a transmembrane protein. In some embodiments, the cell surface binding partner is a receptor.

The terms “binding partner” or “counter-structure” in reference to a protein, such as a receptor, soluble ligand, or to an extracellular domain or portion thereof or affinity-modified variant thereof, refers to at least one molecule (typically a native mammalian protein) to which the referenced protein specifically binds under specific binding conditions. In some aspects, an affinity-modified domain, or an immunomodulatory protein containing an affinity-modified domain, specifically binds to the binding partner of the corresponding domain of the native or wild-type protein but with increased or attenuated affinity. A cell surface binding partner is a binding partner expressed on a mammalian cell. Typically, the cell surface binding partner is a transmembrane protein. In some embodiments, the cell surface binding partner is a receptor, or a ligand of a receptor expressed on and by cells, such as mammalian cells, forming the immunological synapse, for example immune cells.

The term “cis” with reference to binding to cell surface molecules refers to binding to two or more different cell surface molecules, each of which is present on the surface of the same cell. In some embodiments, cis means that the two or more cell surface molecules are exclusively on one or exclusively the other (but not both) of the two mammalian cells forming the IS.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acid substitution groups are: valine-leucine-isoleucine, phenylalanine-tyrosine, lysine-arginine, alanine-valine, glutamate-aspartate, and asparagine-glutamine.

The term “corresponding to” with reference to positions of a protein, such as recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence based on structural sequence alignment or using a standard alignment algorithm, such as the GAP algorithm. By aligning the sequences, one skilled in the art can identify corresponding residues, for example, using conserved and identical amino acid residues as guides.

As used herein, “domain” (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule, such as a cognate binding partner. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed, appropriate software can be employed to identify domains. It is understood that reference to amino acids, including to a specific sequence set forth as a SEQ ID NO used to describe domain organization (e.g. of a TD domain) are for illustrative purposes and are not meant to limit the scope of the embodiments provided. It is understood that polypeptides and the description of domains thereof are theoretically derived based on homology analysis and alignments with similar molecules. Also, in some cases, adjacent N- and/or C-terminal amino acids of a given domain (e.g. TD) also can be included in a sequence, such as to ensure proper folding of the domain when expressed. Thus, the exact locus can vary, and is not necessarily the same for each protein. For example, a specific TD domain, such as specific CRD domain, can be several amino acids (1-10, such as 1, 2, 3, 4, 5, 6, 7, 8, 9 or 10 amino acids) longer or shorter.

The terms “ectodomain,” “extracellular domain,” or “ECD,” which are used interchangeably herein, refer to a region of a membrane protein, such as a transmembrane protein, which lies outside the vesicular membrane (e.g., the space outside of a cell), when a full-length form of the membrane protein is expressed from a cell. For purposes herein, it is understood that reference to the ECD refers to sequences and domains that make up this region and do not require that a protein that contains an ECD is a membrane protein or that the domain is present outside a cell. For example, a soluble immunomodulatory protein can contain ECD sequences of a membrane protein fused to another moiety, such as a multimerization domain, for example an Fc region. Ectodomains often interact with specific ligands or specific cell surface receptors, such as via a binding domain that specifically binds to the ligand or cell surface receptor. Examples of binding domains include cysteine rich domains (CRDs). Ectodomains of members of the TNFR superfamily contain a TD domain (e.g. a CRD domain). Thus, reference to an ECD herein includes a full-length sequence of an ECD of a membrane protein as well as specific-binding fragments thereof containing a CRD that bind to a ligand or cognate binding partner.

The terms “effective amount” or “therapeutically effective amount” refer to a quantity and/or concentration of a therapeutic pharmaceutical formulation or pharmaceutical composition, such as containing an immunomodulatory protein or Fc fusion protein, that when administered ex vivo (by contact with a cell from a patient) or in vivo (by administration into a patient) either alone (i.e., as a monotherapy) or in combination with additional therapeutic agents, yields a statistically significant inhibition of disease progression as, for example, by ameliorating or eliminating symptoms and/or the cause of the disease. An effective amount for treating a disease, condition or disorder, such as an immune system disease, condition or disorder, may be an amount that relieves, lessens, or alleviates at least one symptom or biological response or effect associated with the disease, condition or disorder, prevents progression of the disease, condition or disorder, or improves physical functioning of the patient. In the case of cell therapy, the effective amount is an effective dose or number of cells administered to a patient. In some embodiments the patient is a human patient.

As used herein, “fusion protein” refers to a polypeptide encoded by a nucleic acid sequence containing a coding sequence for two or more proteins, in some cases 2, 3, 4, 5 or more proteins, in which the coding sequences are in the same reading frame such that when the fusion construct is transcribed and translated in a host cell, the protein is produced containing the two or more proteins. Each of the two or more proteins can be adjacent to another protein in the construct or separated by a linker polypeptide that contains, 1, 2, 3, or more, but typically fewer than 20, 15, 10, 9, 8, 7, or 6 amino acids. The protein product encoded by a fusion construct is referred to as a fusion polypeptide. An example of a fusion protein in accord with the provided embodiments is an Fc fusion protein containing an affinity-modified domain (e.g. a variant of a TACI extracellular domain or portion thereof containing a CRD) that is linked to an immunoglobulin Fc domain.

The term “half-life extending moiety” refers to a moiety of a polypeptide fusion or chemical conjugate that extends the half-life of a protein circulating in mammalian blood serum compared to the half-life of the protein that is not so conjugated to the moiety. In some embodiments, half-life is extended by greater than or about 1.2-fold, about 1.5-fold, about 2.0-fold, about 3.0-fold, about 4.0-fold, about 5.0-fold, or about 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety. The half-life refers to the amount of time it takes for the protein to lose half of its concentration, amount, or activity. Half-life can be determined for example, by using an ELISA assay or an activity assay. Exemplary half-life extending moieties include an Fc domain, a multimerization domain, polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), and polyglutamic acid (glutamylation).

An Fc (fragment crystallizable) region or domain of an immunoglobulin molecule (also termed an Fc polypeptide) corresponds largely to the constant region of the immunoglobulin heavy chain, and which, in some cases, is responsible for various functions, including the antibody's effector function(s). The Fc domain contains part or all of a hinge domain of an immunoglobulin molecule plus a CH2 and a CH3 domain. In some cases, for inclusion in a provided fusion protein, all or a portion of the Fc hinge sequence may be deleted. The Fc domain can form a dimer of two polypeptide chains joined by one or more disulfide bonds. In some embodiments, the Fc is a variant Fc that exhibits reduced (e.g. reduced greater than about 30%, 40%, 50%, 60%, 70%, 80%, 90% or more) activity to facilitate an effector function. In some embodiments, reference to amino acid substitutions in an Fc region is by EU numbering system unless described with reference to a specific SEQ ID NO. EU numbering is known and is according to the most recently updated IMGT Scientific Chart (IMGT®, the international ImMunoGeneTics® information system. http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

An immunoglobulin Fc fusion (Fc-fusion), such as an immunomodulatory Fc fusion protein, is a molecule comprising one or more polypeptides operably linked to an Fc region of an immunoglobulin. An Fc-fusion may comprise, for example, an Fc region operably linked to a TACI extracellular domain or portion thereof containing a CRD, including any of the provided affinity-modified variants thereof. An immunoglobulin Fc region may be linked indirectly or directly to the one or more polypeptides. Various linkers are known in the art and can optionally be used to link an Fc to a fusion partner to generate an Fc-fusion. Fc-fusions of identical species can be dimerized to form Fc-fusion homodimers. Fc fusion of non-identical species (e.g. knob into hole engineering) may be used to form Fc-fusion heterodimers. In some embodiments, the Fc is a mammalian Fc such as a murine or human Fc.

The term “host cell” refers to any cell that can be used to express a protein encoded by a recombinant expression vector. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO and related cell lines which grow in serum-free media or CHO strain DX-B 11, which is deficient in DHFR.

The term “immunological synapse” or “immune synapse” (abbreviated IS) as used herein means the interface between a mammalian cell that expresses MHC I (major histocompatibility complex) or MHC II, such as an antigen-presenting cell or tumor cell, and a mammalian lymphocyte such as an effector T cell or Natural Killer (NK) cell.

The term “immunoglobulin” (abbreviated Ig) as used herein is synonymous with the term “antibody” (abbreviated Ab) and refers to a mammalian immunoglobulin protein including any of the five human classes: IgA (which includes subclasses IgA1 and IgA2), IgD, IgE, IgG (which includes subclasses IgG1, IgG2, IgG3, and IgG4), and IgM. The term is also inclusive of immunoglobulins that are less than full-length, whether wholly or partially synthetic (e.g., recombinant or chemical synthesis) or naturally produced, including any fragment thereof containing at least a portion of the variable heavy (VH) chain and/or variable light (VL) chain region of the immunoglobulin molecule that is sufficient to form an antigen binding site and, when assembled, to specifically bind antigen. The antibody also can include all or a portion of the constant region. Such fragments include antigen binding fragment (Fab), variable fragment (Fv) containing VH and VL, the single chain variable fragment (scFv) containing VH and VL linked together in one chain, as well as other antibody V region fragments, such as Fab′, F(ab)2, F(ab′)2, dsFv diabody, Fc, and Fd polypeptide fragments. Hence, it is understood that reference to an antibody herein includes full-length antibody and antigen-binding fragments. The term “antibody” also includes antibody compositions with polyepitopic specificity, multispecific antibodies (e.g., bispecific antibodies), diabodies, and single-chain molecules. Bispecific antibodies, homobispecific and heterobispecific, are included within the meaning of the term. Antibodies include polyclonal antibodies or monoclonal antibodies. “Antibody” also includes synthetic antibodies or recombinantly produced antibodies. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6.

The terms “full-length antibody,” “intact antibody” or “whole antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions, such as antibodies produced from mammalian species (e.g. human, mouse, rat, rabbit, non-human primate, etc.) by antibody secreting B cells and antibodies with the same domains that are produced synthetically. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An antibody fragment comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; diabodies; linear antibodies (see U.S. Pat. No. 5,641,870, Example 2; Zapata et al., Protein Eng. 8(10): 1057-1062 [1995]); single-chain antibody molecules, including single-chain Fvs (scFv) or single-chain Fabs (scFab); antigen-binding fragments of any of the above and multispecific antibodies from antibody fragments.

Fv is composed of one heavy- and one light-chain variable region domain linked by non-covalent association. From the folding of these two domains emanate six complementarity determining regions (CDR) (3 in each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although, in some cases, at a lower affinity than the entire binding site.

dsFv refers to an Fv with an engineered intermolecular disulfide bond, which stabilizes the V_{H}-V_{L} pair.

An Fd fragment is a fragment of an antibody containing a variable domain (V_{H}) and one constant region domain (C_{H}1) of an antibody heavy chain.

A Fab fragment is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

A F(ab′)₂ fragment is an antibody fragment that results from digestion of an immunoglobulin with pepsin at pH 4.0-4.5, or a fragment having the same structure that is produced synthetically, e.g., by recombinant methods. The F(ab′)₂ fragment essentially contains two Fab fragments where each heavy chain portion contains an additional few amino acids including cysteine residues that form disulfide linkages joining the two fragments.

A Fab′ fragment is a fragment containing one half (one heavy chain and one light chain) of the F(ab′)₂ fragment.

An Fd′ fragment is a fragment of an antibody containing one heavy chain portion of a F(ab′)₂ fragment.

An Fv′ fragment is a fragment containing only the V_{H} and V_{L} domains of an antibody molecule.

An scFv fragment refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

Diabodies are dimeric scFv; diabodies typically have shorter peptide linkers than scFvs, and preferentially dimerize.

The term immunological activity as used herein refers to one or more activities of immune cells, such as T cells or B cells, including, for example, activation, cell survival, cell proliferation, cytokine production (e.g. interferon-gamma), cytotoxicity activity, or ability to activate NF-κB pathway or other signaling cascade leading to activation of a transcription factor in the immune cell. Assays to assess immunological activity of immunomodulatory proteins can be compared to control proteins with a known activity.

An immunomodulatory protein or immunomodulatory polypeptide is a protein that modulates immunological activity. By modulation or modulating an immune response is meant that immunological activity is either enhanced or suppressed. Such modulation includes any induction, or alteration in degree or extent, or suppression of immunological activity of an immune cell, such as a B cell or a T cell. For example, soluble Fc fusion proteins herein may suppress immunological activity of B cells. An immunomodulatory protein can be a single polypeptide chain or a multimer (dimers or higher order multimers) of at least two polypeptide chains covalently bonded to each other by, for example, interchain disulfide bonds. Thus, monomeric, dimeric, and higher order multimeric proteins are within the scope of the defined term. Multimeric proteins can be homomultimeric (of identical polypeptide chains) or heteromultimeric (of different polypeptide chains).

As used herein, modification is in reference to modification of a sequence of amino acids of a polypeptide or a sequence of nucleotides in a nucleic acid molecule and includes a change in amino acids or nucleotides, respectively, of the sequence. The amino acid modification or change may be a deletion, insertion, or replacement (substitution) of amino acids or nucleotides, respectively. Methods of modifying a polypeptide are routine to those of skill in the art, such as by using recombinant DNA methodologies.

The term, a multimerization domain refers to a sequence of amino acids that promotes the formation of a multimer of two or more polypeptides. A multimerization domain includes sequences that promote stable interaction of a polypeptide molecule with one or more additional polypeptide molecules, each containing a complementary multimerization domain (e.g. a first multimerization domain and a second multimerization domain), which can be the same or a different multimerization domain. The interactions between complementary multimerization domains, e.g. interaction between a first multimerization domain and a second multimerization domain, form a stable protein-protein interaction to produce a multimer of the polypeptide molecule with the additional polypeptide molecule. In some cases, the multimerization domain is the same and interacts with itself to form a stable protein-protein interaction between two polypeptide chains. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof.

The terms nucleic acid and polynucleotide are used interchangeably to refer to a polymer of nucleic acid residues (e.g., deoxyribonucleotides or ribonucleotides) in either single- or double-stranded form. Unless specifically limited, the terms encompass nucleic acids containing known analogues of natural nucleotides and that have similar binding properties to it and are metabolized in a manner similar to naturally occurring nucleotides. Unless otherwise indicated, a particular nucleic acid sequence also implicitly encompasses conservatively modified variants thereof (e.g., degenerate codon substitutions) and complementary nucleotide sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid or polynucleotide encompasses cDNA or mRNA encoded by a gene.

The terms in operable combination, in operable order and operably linked as used herein refer to the linkage of nucleic acid sequences in such a manner or orientation that the segments are arranged so that they function in concert for their intended purposes. In some embodiments, the term refers to linkage of nucleic acids to produce a nucleic acid molecule capable of directing the transcription of a given gene and/or to produce a desired protein molecule that is functional. For example, segments of a DNA sequence, e.g. a coding sequence and a regulatory sequence(s), are linked in such a way as to permit gene expression when the appropriate molecules (e.g. transcriptional activator proteins) are bound to the regulatory sequence.

The term pharmaceutical composition or pharmaceutical formulation or formulation or TACI-Fc formulation are used interchangeably and refers to a composition or formulation suitable for pharmaceutical use in a mammalian subject, often a human. A pharmaceutical composition or pharmaceutical formulation herein comprises an effective amount of an active agent (e.g., a TACI-Fc fusion protein) and other components as provided herein.

As used herein, the term salt refers to inorganic salts, which include but are not limited to sodium chloride (NaCl), sodium sulfate (Na₂SO₄), sodium thiocyanate (NaSCN), magnesium chloride (MgCl), magnesium sulfate (MgSO₄), ammonium thiocyanate (NH₄SCN), ammonium sulfate ((NH₄)₂SO₄), ammonium chloride (NH₄Cl), calcium chloride (CaCl₂)), calcium sulfate (CaSO₄), zinc chloride (ZnCl₂) and the like, or combinations thereof. Unless otherwise indicated, the protein formulations provided herein are characterized by a substantial absence of added salt. It will be understood by those of skill in the art that the presence of inorganic salts within the presently disclosed formulations that are introduced by pH adjustment are not considered to be added salts and such inorganic salts, if present in a formulation according to the present embodiments. In some embodiments herein, a salt can be added to the pharmaceutical formulations provided herein. In embodiments, a salt or salts is added to pharmaceutical formulations provided herein in the presence of an amino acid in the formulation, such as, for example, proline.

As used herein, the term “surfactant” refers to a pharmaceutically acceptable surface-active agent. Surfactants herein include “non-ionic surfactants” including, without limitation, polysorbates and poloxamers. In some embodiments the poloxamer is poloxamer 188. In some embodiments the polysorbate is polysorbate 20 and/or 80. The use of non-ionic surfactants permits the formulations to be exposed to shear and surface stresses without causing denaturation of the protein in the formulation (e.g., TACI-Fc), and also can reduce protein adsorption on the surfaces during processing and storage. The formulations provided herein include, without limitation, formulations having one or more non-ionic surfactant(s) including, for example, one or more polysorbate(s), such as polysorbate 80 or 20; one or more poloxamers, such as poloxamer 188. Exemplified herein are formulations having a poloxamer, such as a poloxamer 188 (e.g., Kolliphor® P 188 Bio, BASF). Also exemplified herein are formulations containing a polysorbate, such as polysorbate 80 (e.g., Tween® 80). Amounts of surfactants effective to provide stable high concentration protein formulations herein generally are in the range of 0.005% to 0.3% (w/v) of the formulation.

The surfactant in the formulations may be or comprise a poloxamer. “Poloxamer” refers to ABA-type copolymers of poly (ethylene oxide (EO)) (PEO=A) and poly (propylene oxide (PO)) (PPO=B). One exemplary poloxamer is “poloxamer 188,” which is also known as P188 or as poly(ethylene glycol)-block-poly(propylene glycol)-block-poly(ethylene glycol), or as CAS-9003-11-6. The approximate relative amount of PEO and the average molecular weight of the PPO may be indicated in the name of the poloxamer. The generic term “poloxamer” is commonly followed by a numerical value of 3 digits: the first 2 digits, multiplied×100, indicates the MW of the hydrophobic core of propylene glycol, and the last digit, multiplied×10, gives the percentage of the hydrophilic PEG content. Poloxamer 188 contains about 80% ethylene oxide repeats by weight, with two about 75-repeat polyethylene chains (about 6600 g/mol out of an average 8400 g/mol), or in other words it contains about 80% m/m PEO (P 188; 8×10=80%) and an approximate average molecular weight of PPO of 1800 (P 188; 18×100=1800). The poloxamer may have an average molecular weight of PPO of about 1800, or from about 1750 to about 1850, or from about 1700 to about 1900, or from about 1500 to about 2000, as examples. The poloxamer may contain about 80% m/m PEO, or from about 78% to about 82% m/m PEO, or from about 80% to about 85% m/m PEO, as examples. The poloxamer is soluble in water and may be characterized by its hydrophilic lipophilic balance (HLB), which may range, for example, from about 14 to about 18, or from about 15 to about 17, or may be about 16 as in the case of poloxamer 188. The poloxamer may be in admixture with byproduct present in a minor amount, where one such byproduct is A-B diblock copolymer. The poloxamer may be of various grades suitable for pharmaceutical uses, e.g., BASF offers poloxamer 188 as Kolliphor® P 188 Bio and Kolliphor® P 188 Cell Culture, which differ slightly, e.g., by peak molecular weight.

The poloxamer may be characterized in terms of the number of repeating EO and PO units in the copolymer, where a poloxamer of formula (PEO)a-(PPO)b-(PEO)a may have “a” equal to about 65-95 EO units in each PEO and “b” equal to about 20-35 PO units in PPO. The poloxamer may be poloxamer 188, wherein “a” is equal to 75-85 and “b” is equal to 25-30.

A “tonicifying agent” or “tonicity agent” as used herein, refers to a substance that is capable of modifying the osmotic pressure of a formulation or solution. In some embodiments, a tonicifying agent may be added to a formulation to make it “isotonic” with a reference solution or fluid such as a bodily fluid, or alternatively, to make it hypertonic or hypotonic with respect to such a reference solution or fluid.

As used herein, the term “viscosity” refers to the resistance of a liquid formulation to flow, such as when injected through a syringe needle during administration to a patient. Viscosity measurements can be assessed by techniques know in the art. For example, viscosity can be assessed by a cone and plate technique with a Peltier element set at a defined temperature, such as 5° C. to 25° C. as described herein. Typically, a well-defined shear stress gradient is applied to the liquid formulation and the resulting shear rate is measured. The viscosity is the ratio of the shear stress to the shear rate. As used herein, viscosity is expressed in units of cP.

As used herein, a “stable” formulation, such as a stable liquid pharmaceutical formulation is a liquid TACI-Fc formulation with no significant changes observed after storage at a refrigerated temperature (2-8° C.) for at least 12 weeks, such as at least 26 weeks, at least about 26 weeks, at least 36 weeks, at least 52 weeks (1 year), at least 18 months, or at least 24 months, or is one with no significant changes observed after storage at room temperature (25° C.) for at least or about 8 weeks or 12 weeks. In some embodiments a stable TACI-Fc formulation is one where at least about 95% of the TACI-Fc protein in the formulation is in the main species as measured by size exclusion chromatography (SEC) after storage, and/or wherein the formulation comprises no more than about 5% of low and high molecular weight species as measured by size exclusion chromatography after storage at a refrigerated temperature (2-8° C.) for at least 12 weeks, such as at least 26 weeks, at least about 26 weeks, at least 36 weeks, at least 52 weeks (1 year), at least 18 months, or at least 24 months, or is one with no significant changes observed after storage at room temperature (25° C.) for at least or about 8 weeks or 12 weeks. no more than 5% of TACI-Fc homodimer is degraded as measured by size exclusion chromatography (SEC-HPLC) after storage for 52 weeks at refrigerated temperature (2-8° C.) or after storage for eight-weeks at room temperature (25° C.). A stable TACI-Fc formulation, in some embodiments, is one where the protein concentration of the formulation changes by no more than +/−10% after such storage, as measured by methods known in the art such as size exclusion chromatography (SEC-HPLC), imaged capillary isoelectric focusing (icIEF), non-reduced and reduced capillary electrophoresis, subvisible particle assessment, visual inspection, and/or anti-APRIL and anti-BAFF activity.

The terms “polypeptide” and “protein” are used interchangeably herein and refer to a molecular chain of two or more amino acids linked through peptide bonds. The terms do not refer to a specific length of the product. Thus, peptides and oligopeptides, are included within the definition of polypeptide. The terms include post-translational modifications of the polypeptide, for example, glycosylations, acetylations, phosphorylations, and the like. The terms also include molecules in which one or more amino acid analogs or non-canonical or unnatural amino acids are included as can be synthesized or expressed recombinantly using known protein engineering techniques. In addition, proteins can be derivatized as described herein by well-known organic chemistry techniques.

The term “purified” as applied to polypeptides generally denotes a polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is purified. A purified nucleic acid or protein is at least about 35% pure, such as usually at least about 50%, 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a recombinant nucleic acid is one that is made by recombining nucleic acids, e.g., during cloning, affinity modification, DNA shuffling or other well-known molecular biological procedures. A recombinant DNA molecule, is comprised of segments of DNA joined together by means of such molecular biological techniques. The term recombinant protein or recombinant polypeptide as used herein refers to a protein molecule (e.g., an immunomodulatory protein) which is expressed using a recombinant DNA molecule. A recombinant host cell is a cell that contains and/or expresses a recombinant nucleic acid or that is otherwise altered by genetic engineering, such as by introducing into the cell a nucleic acid molecule encoding a recombinant protein, such as an immunomodulatory protein provided herein. Transcriptional control signals in eukaryotes comprise promoter and enhancer elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest.

The term “recombinant expression vector” as used herein refers to a DNA molecule containing a desired coding sequence (e.g., encoding an immunomodulatory protein) and appropriate nucleic acid sequences necessary for the expression of an operably linked coding sequence in a particular cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the recombinant expression vector, operably linked to the coding sequence so that the expressed protein can be secreted by the recombinant host cell, such as for its expression as a secretable protein or for more facile isolation or purification of the immunomodulatory protein from the cell, if desired. The term includes the vector as a self-replicating nucleic acid structure as well as the vector incorporated into the genome of a host cell into which it has been introduced. Among the vectors are viral vectors, such as lentiviral vectors.

The term “sequence identity” as used herein refers to the sequence identity between genes or proteins at the nucleotide or amino acid level, respectively. Sequence identity is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. Methods for the alignment of sequences for comparison are well known in the art, such methods include GAP, BESTFIT, BLAST, BLAST-2, ALIGN or Megalign (DNASTAR) software, FASTA and TFASTA. The BLAST algorithm calculates percent sequence identity and performs a statistical analysis of the similarity between the two sequences. The software for performing BLAST analysis is publicly available through the National Center for Biotechnology Information (NCBI) website. In some cases, a percent sequence identity can be determined as the percentage of amino acid residues (or nucleotide residues) in a candidate sequence that are identical with the amino acid residues (or nucleotide residues) in a reference sequence, after aligning the sequences and introducing gaps, if necessary, to achieve the maximum percent sequence identity. Reference to sequence identity includes sequence identity across the full length of each of the sequences being compared. Those skilled in the art can determine appropriate parameters for aligning sequences, including any algorithms needed to achieve maximal alignment over the full length of the sequences being compared.

The term “soluble” as used herein in reference to proteins means that the protein is not a membrane protein or is not anchored in a cell membrane. A protein can be constructed as a soluble protein by inclusion of only an extracellular domain or a portion thereof and without a transmembrane domain. In some cases, solubility of a protein can be improved by linkage or attachment, directly or indirectly via a linker, to an Fc domain or other half-life extending molecule, which, in some cases, also can improve the stability and/or half-life of the protein. In some aspects, a soluble protein is an Fc fusion protein.

The term “specifically binds” as used herein means the ability of a protein, under specific binding conditions, to bind to a target protein such that its affinity or avidity is at least 10 times as great, but optionally 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to more than one target molecule. In some cases, a specifically binding protein may bind to a protein that has similarity in structural conformation with the target protein (e.g., paralogs or orthologs). Those of skill will recognize that specific binding to a molecule having the same function in a different species of animal (i.e., ortholog) or to a molecule having a substantially similar epitope as the target molecule (e.g., paralog) is possible and does not detract from the specificity of binding which is determined relative to a statistically valid collection of unique non-targets (e.g., random polypeptides). Thus, an immunomodulatory protein of the invention may specifically bind to more than one distinct species of target molecule due to cross-reactivity. Solid-phase ELISA immunoassays, ForteBio Octet or Biacore measurements can be used to determine specific binding between two proteins. Generally, interactions between two binding proteins have dissociation constants (Kd) less than about 1×10⁻⁵ M, and often as low as about 1×10⁻¹² M. In certain aspects of the present disclosure, interactions between two binding proteins have dissociation constants of less than about 1×10⁻⁶ M, 1×10⁻⁷ M, 1×10⁻⁸ M, 1×10⁻⁹ M, 1×10⁻¹⁰ M, or 1×10⁻¹¹ M or less.

The term “specific binding fragment” or “fragment” as used herein in reference to a protein means a polypeptide that is shorter than a full-length protein or a specific domain or region thereof and that specifically binds in vitro and/or in vivo to a binding partner of the full-length protein or of the specific domain or region. A specific finding fragment is in reference to a fragment of a full-length extracellular domain of a polypeptide or a binding domain of a polypeptide, but that still binds to a binding partner of the binding domain. For example, a specific binding fragment is in reference to a fragment of an extracellular domain of a full-length TNFR family member or a full-length TNFR domain (TD) thereof (e.g. CRD), but that still binds to a binding partner of the TNFR family member or of a CRD of an TNFR family member. In some embodiments, the specific binding fragment is at least about 20%, 30%, 40%, 50%, 60%, 70%, 75%, 80%, 85%, 90%, 95%, 96%, 97%, 98%, or 99% the sequence length of the full-length sequence of the extracellular domain or of a domain or region of the extracellular domain. In some embodiments, the specific binding fragment can have an amino acid length of at least 50 amino acids, such as at least 60, 70, 80, 90, 100, or 110 amino acids. In some embodiments, the specific binding fragment includes the CRD1 and/or CRD2 domain. In some embodiments, the specific binding fragment includes the CRD2 domain.

As used herein, an “injection device” is an apparatus for administration of a formulation herein. In embodiments herein, an injection device contains a needle for administration. In other embodiments here, an injection device is a needle free injection device, such as, for example, a needle free autoinjector. An injection device can be used for administration of a formulation to a subject, such as, for example, for subcutaneous or intravenous administration. Examples of an injection device are a syringe or a syringe/autoinjector combination. In some embodiments herein, an autoinjector contains a syringe loaded into the autoinjector.

As used herein, a “subject” is a mammal, such as a human or other animal, and typically is human. The subject can be male or female and can be any suitable age, including infant, juvenile, adolescent, adult, and geriatric subjects.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

The term “TNF receptor” superfamily or TNFRSF as used herein means the group of cell surface cytokine receptors that are all type I (N-terminus extracellular) transmembrane glycoproteins that contain one to six cysteine rich domains (CRD) in their extracellular domain. Molecules are categorized as members of this superfamily based on the shared structural features that include the one or more cysteine rich domain (CRD) present in their N-terminal extracellular region, which often play a role in protein binding of their cognate binding partner or ligand. A TNFRSF protein may have only one or several CRDs (e.g. CRD1, CRD2, etc.). Typically, ECD or ectodomain of TNFRSF members contain between 1 and 6 pseudorepeats of CRDs. For example, BAFF-receptor and BCMA each contain one CRD while TACI contains two CRDs (CRD1 and CRD2). TNFRSF members are usually trimeric or multimeric complexes that are stabilized by their intracysteine disulfide bonds. Binding of TNFRSF proteins to their ligands facilitates various biological activities in cells, such as the induction of apoptotic cell death or cell survival and proliferation.

The term “TD” refers to a structural domain or domains of TNFRSF proteins or of TNF family ligands. For example, a TD of a TNFRSF protein is a cysteine-rich domain (CRD) module of about 40 amino acids containing six (6) conserved cysteines. Hence, reference to CRD also can be used interchangeably with the term TD in reference to a TD of a TNFRSF protein. The six cysteines are involved in formation of intrachain disulphide bonds. The extracellular domain (ECD) of TNFRSF members contains one or more CRD domains; hence, the term TD is also used with reference to the ECD of such protein molecules. Reference to a variant TD (vTD) refers to a variant or modified sequence of a TD.

The term “trans” with reference to binding to cell surface molecules refers to binding to two different cell surface molecules, each of which is present on the surface of a different cell. In some embodiments, trans means that with respect to two different cell surface molecules, the first is exclusively present on one of the two mammalian cells forming the IS and the second is present exclusively on the second of the two mammalian cells forming the IS.

The term “transmembrane protein” as used herein means a membrane protein that substantially or completely spans a lipid bilayer such as those lipid bilayers found in a biological membrane such as a mammalian cell, or in an artificial construct such as a liposome. The transmembrane protein comprises a transmembrane domain (transmembrane domain) by which it is integrated into the lipid bilayer and by which the integration is thermodynamically stable under physiological conditions. Transmembrane domains are generally predictable from their amino acid sequence via any number of commercially available bioinformatics software applications on the basis of their elevated hydrophobicity relative to regions of the protein that interact with aqueous environments (e.g., cytosol, extracellular fluid). A transmembrane domain is often a hydrophobic alpha helix that spans the membrane. A transmembrane protein can pass through both layers of the lipid bilayer once or multiple times.

The terms “treating,” “treatment,” or “therapy” of a disease, condition or disorder as used herein mean slowing, stopping or reversing the disease or disorders progression, as evidenced by decreasing, cessation or elimination of either clinical or diagnostic symptoms, by administration of a formulation or protein or engineered cells of the present invention either alone or in combination with another compound as described herein. Treating, treatment, or therapy also means a decrease in the severity of symptoms in an acute or chronic disease, condition or disorder or a decrease in the relapse rate as for example in the case of a relapsing or remitting autoimmune disease course or inflammatory condition or a decrease in inflammation in the case of an inflammatory aspect of an autoimmune disease or inflammatory condition. The terms “preventing,” “prophylaxis,” or “prevention” of a disease, condition or disorder as used in the context of this invention refers to the administration of a formulation or protein or engineered cells of the present invention, either alone or in combination with another compound, to prevent the occurrence or onset of a disease, condition or disorder or of some or all of the symptoms of a disease, condition or disorder or to lessen the likelihood of the onset of a disease, condition or disorder.

The term “variant” (also modified or mutant, which can be used interchangeably) as used in reference to a variant protein or polypeptide means a protein, such as a mammalian (e.g., human or murine) protein created by human intervention. The variant is a polypeptide having an altered or modified amino acid sequence, such as by one or more amino acid substitutions, deletions, additions or combinations thereof, relative to an unmodified or wild-type protein or to a domain thereof. A variant polypeptide can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions. A variant polypeptide generally exhibits at least about 50%, 60%, 70%, 80%, 85%, 86%, 87%, 88%, 89%, 90%, 91%, 92%, 93%, 94%, 95%, 96%, 97%, 98%, 99% or more sequence identity to a corresponding form of a wild-type or unmodified protein, such as a mature sequence thereof (lacking the signal sequence) or a portion thereof containing the extracellular domain or an binding domain thereof. Non-naturally occurring amino acids as well as naturally occurring amino acids are included within the scope of permissible substitutions or additions. A variant protein is not limited to any particular method of making and includes, for example, chemical synthesis, recombinant DNA techniques, or combinations thereof. A variant protein of the invention specifically binds to at least one or more binding partners. In some embodiments, the altered amino acid sequence results in an altered (i.e., increased or decreased) binding activity, such as binding affinity or avidity, to the one or more binding partners. A variant protein may thus be an affinity-modified protein as described herein.

The term “wild-type” or “natural” or “native,” which are used interchangeably, as used herein is used in connection with biological materials such as nucleic acid molecules, proteins, host cells, and the like, which are found in nature and not modified by human intervention.

### II. PHARMACEUTICAL FORMULATIONS

Provided herein are formulations containing a fusion protein comprising a TACI polypeptide and an Fc region (TACI-Fc fusion protein), such as any TACI-Fc fusion protein described herein. In provided embodiments, the TACI polypeptide is an extracellular domain or portion containing a TNF receptor domain (TD, e.g, CRD2) of TACI or is a variant TACI polypeptide thereof containing one or more amino acid substitutions. Provided embodiments include methods and uses of a formulation comprising a variant TACI-Fc fusion protein that is an Fc fusion protein of a variant TACI extracellular domain or portion containing a TD (e.g., CRD2) that simultaneously inhibits the BAFF and APRIL cytokines. Provided embodiments also include methods and uses of a formulation comprising a wild-type TACI-Fc fusion protein that is an Fc fusion protein of a wild-type TACI extracellular domain or portion that contains a TD (e.g. CRD2) that inhibits BAFF or APRIL or simultaneously inhibits the BAFF and APRIL cytokines. The formulations contain a TACI-Fc fusion biologic for the treatment of serious autoimmune and/or inflammatory diseases, including B cell-related diseases such as any described in Section V.

In some embodiments, pharmaceutical formulations comprise a therapeutically effective amount of a TACI-Fc fusion protein, such as any amount described herein. Also provided are any of the provided pharmaceutical formulations for use in treating an autoimmune or inflammatory disease in a patient in need thereof, such as any uses for treating such diseases or conditions as described in Section V. Also provided are methods of treating an autoimmune or inflammatory disease in a patient in need thereof by administering any of such pharmaceutical formulations, such as for treating any disease or conditions as described in Section V.

As described herein, among the provided formulations are TACI-Fc formulations that contain a high concentration of TACI-Fc protein. The formulations contain a combination of excipients such that the TACI-Fc in the formulation is stable after prolonged storage.

### A. TACI-Fc Protein

The provided formulations are prepared with any TACI-Fc fusion protein. The TACI-Fc fusion protein may contain an extracellular domain (ECD) or portion of the ECD of the TACI receptor, or a variant thereof, that bind to at least one TACI cognate binding partner. Also provided herein are formulations containing variant TACI polypeptides that exhibit altered (e.g. increased) binding activity or affinity for one or more of a TACI cognate binding partner. In some embodiments, the TACI cognate binding partner is one or more of BAFF or APRIL or is a BAFF/APRIL heterotrimer. The provided TACI-Fc fusion proteins are soluble fusion proteins in which the TACI extracellular domain or portion thereof, or variant thereof, is linked to an immunoglobulin Fc. In some embodiments, provided is a formulation containing a TACI-Fc fusion protein containing (1) a TACI polypeptide composed of the extracellular domain of the TACI receptor or a portion thereof, or a variant TACI polypeptide thereof, that binds to at least one TACI cognate binding partner, and (2) an Fc domain. The TACI polypeptide or variant TACI polypeptide can be linked directly or indirectly (e.g. via a peptide linker) to the Fc domain.

TACI is a tumor necrosis factor receptor family member characterized by having an extracellular domain (ECD) containing cysteine-rich pseudo-repeat domains (CRDs). TACI is a membrane bound receptor, which has an extracellular domain containing two cysteine-rich pseudo-repeats (CRD1 and CRD2), a transmembrane domain and a cytoplasmic domain that interacts with CAML (calcium-modulator and cyclophilin ligand), an integral membrane protein located at intracellular vesicles which is a co-inducer of NF-AT activation when overexpressed in Jurkat cells. TACI is associated with B cells and a subset of T cells. The TACI receptor binds two members of the tumor necrosis factor (TNF) ligand family. One ligand is designated BAFF (B cell Activating Factor of the TNF Family), and also is variously designated as ZTNF4, “neutrokine-α,” “BLyS,” “TALL-1,” and “THANK” (Yu et al., international publication No. WO98/18921 (1998), Moore et al., Science 285:269 (1999); Mukhopadhyay et al., J. Biol. Chem. 274:15978 (1999); Schneider et al., J. Exp. Med. 189:1747 (1999); Shu et al., J. Leukoc. Biol. 65:680 (1999)). The other ligand has been designated as APRIL, and also is variously designated as “ZTNF2” and “TNRF death ligand-1” (Hahne et al., J. Exp. Med. 188:1185 (1998); Kelly et al., Cancer Res. 60:1021 (2000)). Both ligands are also bound by the B-cell maturation receptor (BCMA) (Gross et al., Nature 404:995 (2000)). Binding of TACI receptor to its ligands BAFF or APRIL stimulates B cell responses, including T cell-independent B cell antibody responses, isotype switching, and B cell homeostasis.

The amino acid sequence of full-length TACI is set forth in SEQ ID NO:88. The protein is a type III membrane protein and lacks a signal peptide; following expression in eukaryotic cells the N-terminal methionine is removed. In some embodiments, a mature TACI protein does not contain the N-terminal methionine as set forth in SEQ ID NO:88. The extracellular domain of TACI (amino acid residues 1-166 of SEQ ID NO:88; ECD set forth in SEQ ID NO:122) contains two cysteine rich domain (CRDs, hereinafter also called a tumor necrosis family receptor domain or TD), each of which exhibit affinity for binding to BAFF and APRIL. The first cysteine rich domain (CRD1) contains amino acid residues 34-66 of the sequence set forth in SEQ ID NO:122. The second cysteine rich domain (CRD2) corresponds to amino acids 71-104 of the sequence set forth in SEQ ID NO:122. TACI also contains a stalk region of about 60 amino acids following the second cysteine repeat in the extracellular domain, corresponding to amino acid residues 105-165 of the sequence set forth in SEQ ID NO:122.

The provided formulations contain a TACI-Fc fusion protein containing a wild-type or variant TACI polypeptide. In some embodiments, for production of the TACI-Fc fusion protein, a nucleic acid molecule encoding a single polypeptide chain of the TACI-Fc fusion protein is inserted into an appropriate expression vector. The resulting TACI-Fc fusion protein can be expressed in host cells transformed with the expression vector, where assembly between Fc domains occurs by interchain disulfide bonds formed between the Fc moieties to yield dimeric, such as divalent, TACI-Fc fusion proteins. The resulting Fc fusion proteins can be easily purified by affinity chromatography over Protein A or Protein G columns. In embodiments, when produced and expressed from a cell, the provided TACI-Fc fusion proteins (e.g. variant TACI-Fc fusion protein) is a homodimer containing two identical polypeptide chains.

Among the provided formulations are those that provide a TACI-Fc fusion protein for neutralizing activity and suppression or reduction of B cell responses. In some embodiments, the TACI-Fc fusion protein provides improved neutralizing activity and suppression or reduction of B cell responses. In some embodiments, the improved activity is mediated by increased or improved binding or interaction of the provided TACI-Fc fusion protein with BAFF and/or APRIL. The provided TACI-Fc fusion proteins block or antagonize interactions of BAFF or APRIL, such as homotrimers of BAFF or APRIL, heterotrimers of BAFF/APRIL or BAFF 60mers, with a cognate B cell stimulatory receptor, and thereby neutralize activity of BAFF and/or APRIL ligands. In some embodiments, the provided TACI-Fc fusion proteins reduce one or more B cell response or activity, including the ability of B cells to produce immunoglobulins. In some embodiments, the provided TACI-Fc fusion proteins (e.g. TACI-Fc fusion protein), when administered to a subject, reduce circulating serum immunoglobulins. In some embodiments, the provided TACI-Fc fusion proteins reduce one or more of B cell maturation, differentiation and proliferation.

In some embodiments, provided TACI-Fc (e.g. variant TACI-Fc) fusion proteins, such as homodimers thereof, exhibit an IC₅₀ for neutralizing BAFF of less than 400 pM. In some embodiments, the IC50 for neutralizing BAFF is between 1 pM and 400 pM, such as between 10 pM and 300 pM, between 10 pM and 200 pM, between 10 pM and 100 pM, between 10 pM and 50 pM, between 10 pM and 20 pM, between 20 pM and 400 pM, between 20 pM and 300 pM, between 20 pM and 200 pM, between 20 pM and 100 pM, between 20 pM and 50 pM, between 50 pM and 400 pM, between 50 pM and 300 pM, between 50 pM and 200 pM, between 50 pM and 100 pM, between 100 pM and 400 pM, between 100 pM and 300 pM, between 100 pM and 200 pM, between 200 pM and 400 pM, between 200 pM and 300 pM, or between 300 pM and 400 pM. In some embodiments, the IC₅₀ for neutralizing BAFF is at or about 10 pM, 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM or 100 pM or any value between any of the foregoing.

In some embodiments, provided TACI-Fc (e.g. variant TACI-Fc) fusion proteins, such as homodimers thereof, exhibits an IC₅₀ for neutralizing APRIL of less than 400 pM. In some embodiments, the IC50 for neutralizing APRIL is between 0.5 pM and 100 pM, such as between 0.5 pM and 50 pM, between 0.5 pM and 25 pM, between 0.5 pM and 10 pM, between 0.5 pM and 5 pM, between 0.5 pM and 1 pM, between 1 pM and 100 pM, between 1 pM and 50 pM, between 1 pM and 25 pM, between 1 pM and 10 pM, between 1 pM and 5 pM, between 5 pM and 100 pM, between 5 pM and 50 pM, between 5 pM and 25 pM, between 5 pM and 10 pM, between 10 pM and 100 pM, between 10 pM and 50 pM, between 10 pM and 25 pM, or between 25 pM and 100 pM, between 25 pM and 50 pM, or between 50 pM and 100 pM. In some embodiments, the IC₅₀ for neutralizing APRIL is at or about 0.5 pM, 0.75 pM, 1 pM, 2 pM, 3 pM, 4 pM, 5 pM, 6 pM, 7 pM, 8 pM, 9 pM, 10 pM, 11 pM, 12 pM, 13 pM, 14 pM, 15 pM, 20 pM or 25 pM or any value between any of the foregoing.

In some embodiments herein, the TACI-Fc (e.g. variant TACI-Fc) fusion proteins, such as homodimers thereof, provided in the formulations herein exhibit neutralizing activity of BAFF and APRIL after storage. In embodiments, the TACI-Fc (e.g. variant TACI-Fc) fusion proteins, such as homodimers thereof, provided in the formulations herein exhibit neutralizing activity of BAFF and APRIL after storage of up to 36 months, such as up to 24 months at refrigerated temperatures (e.g, 2-8° C.) and/or up to 6 months, such as up to 3 months or up to 6 months, at elevated temperatures (e.g., 30° C.). In embodiments herein, the TACI-Fc (e.g. variant TACI-Fc) fusion proteins, such as homodimers thereof, provided in the formulations herein exhibit neutralizing activity of BAFF and APRIL after storage of up to 36 months, such as up to 24 months at refrigerated temperatures (e.g, 2-8° C.) and/or up to 6 months, such as up to 3 months or up to 6 months, at elevated temperatures (e.g., 30° C.) that is at least or at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more compared to the activity prior to storage. In embodiments herein, the TACI-Fc (e.g. variant TACI-Fc) fusion proteins, such as homodimers thereof, provided in the formulations herein exhibit neutralizing activity of BAFF and APRIL after storage for up to 52 weeks at 2-8° C., such as, for example, 5° C., that is at least or at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more compared to the activity prior to storage. In embodiments herein, the TACI-Fc (e.g. variant TACI-Fc) fusion proteins, such as homodimers thereof, provided in the formulations herein exhibit neutralizing activity of BAFF and APRIL after storage for up to 104 weeks at 2-8° C., such as, for example, 5° C., that is at least or at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more compared to the activity prior to storage. In embodiments herein, the TACI-Fc (e.g. variant TACI-Fc) fusion proteins, such as homodimers thereof, provided in the formulations herein exhibit neutralizing activity of BAFF and APRIL after storage for up to 3 years at 2-8° C., such as, for example, 5° C., that is at least or at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more compared to the activity prior to storage. In embodiments herein, the TACI-Fc (e.g. variant TACI-Fc) fusion proteins, such as homodimers thereof, provided in the formulations herein exhibit neutralizing activity of BAFF and APRIL after storage for up to 12 weeks at about 25° C. that is at least or at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more compared to the activity prior to storage. In some embodiments, the TACI-Fc (e.g. variant TACI-Fc) fusion proteins, such as homodimers thereof, provided in the formulations herein exhibit neutralizing activity of BAFF and APRIL after storage for up to 52 weeks at 2-8° C., such as, for example, 5° C. that is at least 95% compared to the activity prior to storage. In some embodiments, the TACI-Fc (e.g. variant TACI-Fc) fusion proteins, such as homodimers thereof, provided in the formulations herein exhibit neutralizing activity of BAFF and APRIL after storage for up to 3 years at 2-8° C., such as, for example, 5° C. that is at least 95% compared to the activity prior to storage.

### 1. TACI Polypeptides

In some embodiments, the provided formulations comprise a TACI-Fc fusion protein comprising a variant TACI polypeptide. In some embodiments, the provided formulations comprise a TACI-Fc fusion protein comprising a wild-type TACI polypeptide. Exemplary TACI polypeptides in the TACI-Fc fusion proteins are described below.

In some embodiments, the variant TACI polypeptide is a wild-type TACI sequence or is a portion thereof that contains one or both CRDs. In some embodiments, the TACI polypeptide is or comprises the extracellular domain (ECD) of TACI or a portion thereof containing one or both CRD domains. In some embodiments, the extracellular domain of a reference (e.g., unmodified) TACI polypeptide comprises a CRD1 and CRD2. In some embodiments, the TACI polypeptide is a soluble polypeptide and lacks a transmembrane domain.

In some embodiments, the TACI polypeptide sequence is a mammalian TACI sequence. In some embodiments, the TACI polypeptide sequence can be a mammalian TACI that includes, but is not limited to, human, mouse, cynomolgus monkey, or rat. In some embodiments, the TACI polypeptide sequence is human. The extracellular domain of an exemplary human TACI sequence is set forth in SEQ ID NO:122.

In some embodiments, the TACI polypeptide sequence has (i) the sequence of amino acids set forth in SEQ ID NO:122 or a sequence thereof that lacks the N-terminal methionine, (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:122 and that binds to APRIL, BAFF or an APRIL/BAFF heterotrimer, or (iii) is a fragment or portion of (i) or (ii) containing a CRD1 and/or CRD2, in which the portion binds to APRIL, BAFF or an APRIL/BAFF heterotrimer. In some embodiments, the TACI polypeptide sequence lacks the N-terminal methionine as set forth in SEQ ID NO: 122.

TACI Extracellular Domain (ECD):
SEQ ID NO: 122
MSGLGRSRRGGRSRVDQEERFPQGLWTGVAMRSCPEEQYWDPLLGTCMSC
KTICNHQSQRTCAAFCRSLSCRKEQGKFYDHLLRDCISCASICGQHPKQC
AYFCENKLRSPVNLPPELRRQRSGEVENNSDNSGRYQGLEHRGSEASPAL
PGLKLSADQVALVYST

In some embodiments, the TACI polypeptide sequence is an extracellular domain sequence of TACI that is a portion of the ECD that contains an N-terminal deletion relative to the sequence of amino acids set forth in SEQ ID NO:122. In some embodiments, the N-terminal deletion is deletion of N-terminal amino acid residues 1-28 corresponding to residues set forth in SEQ ID NO:122. In some embodiments, the N-terminal deletion is deletion of N-terminal amino acid residues 1-29 corresponding to residues set forth in SEQ ID NO:122. In some embodiments, the N-terminal deletion is deletion of N-terminal amino acid residues 1-30 corresponding to residues set forth in SEQ ID NO:122. In some embodiments, the N-terminal deletion is deletion of N-terminal amino acid residues 1-31 corresponding to residues set forth in SEQ ID NO:122. In some embodiments, the N-terminal deletion is deletion of N-terminal amino acid residues 1-32 corresponding to residues set forth in SEQ ID NO:122. In some embodiments, the N-terminal deletion is deletion of N-terminal amino acid residues 1-33 corresponding to residues set forth in SEQ ID NO:122.

Provided herein is an immunomodulatory protein (e.g. TACI-Fc fusion protein) containing a TACI polypeptide that is a portion of the TACI extracellular domain (ECD) region that contains the CRD2, with a deletion of the N-terminal region and CRD1 and deletion of one or more residues of the stalk portion of the TACI extracellular domain, e.g. relative to the sequence of amino acids set forth in SEQ ID NO:122. In some embodiments, the portion of the TACI extracellular domain that contains the CRD2 includes amino acid residues 71-104 corresponding to residues set forth in SEQ ID NO:122. In provided embodiments, the TACI polypeptide of the immunomodulatory protein contains deletion of N-terminal amino acid residues 1-66 corresponding to residues set forth in SEQ ID NO:122. In provided embodiments, the TACI polypeptide of the immunomodulatory protein contains deletion of N-terminal amino acid residues 1-67 corresponding to residues set forth in SEQ ID NO:122. In provided embodiments, the TACI polypeptide of the immunomodulatory protein contains deletion of N-terminal amino acid residues 1-68 corresponding to residues set forth in SEQ ID NO:122. In provided embodiments, the TACI polypeptide of the immunomodulatory protein contains deletion of N-terminal amino acid residues 1-69 corresponding to residues set forth in SEQ ID NO:122. In provided embodiments, the TACI polypeptide of the immunomodulatory protein contains deletion of N-terminal amino acid residues 1-70 corresponding to residues set forth in SEQ ID NO:122. In embodiments of any such embodiments, the TACI polypeptide of the immunomodulatory protein lacks one or more contiguous C-terminal amino acid residues beginning at residue 105 and up to or including amino acid residue 166 corresponding to residues of the ECD sequence set forth in SEQ ID NO:122. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61 or 62 residues of the ECD sequence are deleted.

In some embodiments, the TACI polypeptide sequence contains an ECD portion having a contiguous sequence of amino acids that includes the CRD1 and/or CRD2 (e.g. CRD1 and CRD2 or CRD2 only) and only a segment or portion of the stalk sequence. Suitable stalk segments include one or more amino acids of amino acid residues 105 to 154 of SEQ ID NO:122. For example, the stalk segment can consist of the following with reference to SEQ ID NO:122: amino acid residue 105, amino acid residues 105 to 106, amino acid residues 105 to 107, amino acid residues 105 to 108, amino acid residues 105 to 109, amino acid residues 105 to 110, amino acid residues 105 to 111, amino acid residues 105 to 112, amino acid residues 105 to 113, amino acid residues 105 to 114, amino acid residues 105 to 115, amino acid residues 105 to 116, amino acid residues 105 to 117, amino acid residues 105 to 118, amino acid residues 105 to 119, amino acid residues 105 to 120, amino acid residues 105 to 121, amino acid residues 105 to 122, amino acid residues 105 to 123, amino acid residues 105 to 124, amino acid residues 105 to 125, amino acid residues 105 to 126, amino acid residues 105 to 127, amino acid residues 105 to 128, amino acid residues 105 to 129, amino acid residues 105 to 130, amino acid residues 105 to 131, amino acid residues 105 to 132, amino acid residues 105 to 133, amino acid residues 105 to 134, amino acid residues 105 to 135, amino acid residues 105 to 136, amino acid residues 105 to 137, amino acid residues 105 to 138, amino acid residues 105 to 139, amino acid residues 105 to 140, amino acid residues 105 to 141, amino acid residues 105 to 142, amino acid residues 105 to 143, amino acid residues 105 to 144, amino acid residues 105 to 145, amino acid residues 105 to 146, amino acid residues 105 to 147, amino acid residues 105 to 148, amino acid residues 105 to 149, amino acid residues 105 to 150, amino acid residues 105 to 151, amino acid residues 105 to 152, amino acid residues 105 to 153, and amino acid residues 105 to 154.

In some embodiments, the TACI polypeptide sequence lacks or is mutated in one or more potential furin cleavage sites. In some cases, the TACI polypeptide sequence is an ECD or portion that in which the arginine residue at position 119 is mutated, e.g. R119G. In some cases, the reference (e.g. unmodified) TACI sequence is an ECD or portion that in which the glutamine residue at position 121 is mutated, e.g. Q121P. In some cases, the reference (e.g. unmodified) TACI sequence is an ECD or portion that in which the arginine residue at position 122 is mutated, e.g. R122Q.

In some embodiments, the reference TACI sequence is a TACI ECD sequence as set forth in international PCT publication No. WO2000/067034, WO2002/094852 or WO2008/154814.

In some embodiments, the TACI polypeptide sequence is a TACI ECD sequence that has or consists of the sequence set forth in SEQ ID NO:131.

TACI ECD (CRD1/CRD2):
SEQ ID NO: 131
SRVDQEER FPQGLWTGVA MRSCPEEQYW DPLLGTCMSCKTICNHQSQ
RTCAAFCRSLS CRKEQGKFYD HLLRDCISCA SICGQHPKQCAYFCEN
KLRSPVNLPPEL

In some embodiments, the TACI polypeptide sequence is a TACI ECD sequence that has or consists of the sequence set forth in SEQ ID NO:130.

TACI ECD (CRD1/CRD2):
SEQ ID NO: 130
AMRSCPEEQYWDPLLGTCMSCKTICNHQSQRTCAAFCRSLSCRKEQGKFY
DHLLRDCISCASICGQHPKQCAYFCENKLRS

In some embodiments, the TACI polypeptide sequence is a TACI ECD sequence that has or consists of the sequence set forth in SEQ ID NO:1 (encoded by the sequence of nucleotides set forth in SEQ ID NO:36).

TACI ECD (CRD1/CRD2):
SEQ ID NO: 1
VAMRSCPEEQYWDPLLGTCMSCKTICNHQSQRTCAAFCRSLSCRKEQGK
FYDHLLRDCISCASICGQHPKQCAYFCENKLRS

In some embodiments, an immunomodulatory protein (e.g. TACI-Fc fusion protein) provided herein has a TACI polypeptide with a sequence that contains an ECD portion having a contiguous sequence of amino acids of a TACI ECD that includes the CRD2 (e.g. residues 71-104 with reference to SEQ ID NO:122), but with a deletion of the N-terminal region and CRD1 and deletion of one or more residues of the stalk portion of the TACI extracellular domain, e.g. relative to the sequence of amino acids set forth in SEQ ID NO:122. For example, the TACI ECD portion can consist of the following with reference to amino acid residues set forth in SEQ ID NO:122: amino acid residues 67 to 118, amino acid residues 67 to 117, amino acid residues 67 to 116, amino acid residues 67 to 115, amino acid residues 67 to 114, amino acid residues 67 to 113, amino acid residues 67 to 112, amino acid residues 67 to 111, amino acid residues 67 to 110, amino acid residues 67 to 109, amino acid residues 67 to 108, amino acid residues 67 to 107, amino acid residues 67 to 106, amino acid residues 67 to 105, or amino acid residues 67 to 104. In some examples, the TACI ECD portion can consist of the following with reference to residues set forth in SEQ ID NO: 122: amino acid residues 68 to 118, amino acid residues 68 to 117, amino acid residues 68 to 116, amino acid residues 68 to 115, amino acid residues 68 to 114, amino acid residues 68 to 113, amino acid residues 68 to 112, amino acid residues 68 to 111, amino acid residues 68 to 110, amino acid residues 68 to 109, amino acid residues 68 to 108, amino acid residues 68 to 107, amino acid residues 68 to 106, amino acid residues 68 to 105, or amino acid residues 68 to 104. In some examples, the TACI ECD portion can consist of the following with reference to residues set forth in SEQ ID NO: 122: amino acid residues 69 to 118, amino acid residues 69 to 117, amino acid residues 69 to 116, amino acid residues 69 to 115, amino acid residues 69 to 114, amino acid residues 69 to 113, amino acid residues 69 to 112, amino acid residues 69 to 111, amino acid residues 69 to 110, amino acid residues 69 to 109, amino acid residues 69 to 108, amino acid residues 69 to 107, amino acid residues 69 to 106, amino acid residues 69 to 105, or amino acid residues 69 to 104. In some examples, the TACI ECD portion can consist of the following with reference to residues set forth in SEQ ID NO: 122: amino acid residues 70 to 118, amino acid residues 70 to 117, amino acid residues 70 to 116, amino acid residues 70 to 115, amino acid residues 70 to 114, amino acid residues 70 to 113, amino acid residues 70 to 112, amino acid residues 70 to 111, amino acid residues 70 to 110, amino acid residues 70 to 109, amino acid residues 70 to 108, amino acid residues 70 to 107, amino acid residues 70 to 106, amino acid residues 70 to 105, or amino acid residues 70 to 104. In some examples, the TACI ECD portion can consist of the following with reference to residues set forth in SEQ ID NO: 122: amino acid residues 71 to 118, amino acid residues 71 to 117, amino acid residues 71 to 116, amino acid residues 71 to 115, amino acid residues 71 to 114, amino acid residues 71 to 113, amino acid residues 71 to 112, amino acid residues 71 to 111, amino acid residues 71 to 110, amino acid residues 71 to 109, amino acid residues 71 to 108, amino acid residues 71 to 107, amino acid residues 71 to 106, amino acid residues 71 to 105, or amino acid residues 71 to 104. Any of the above TACI ECD sequences also can be a TACI reference sequence in accord with the immunomodulatory proteins provided herein, in which such immunomodulatory proteins contain a variant TACI polypeptide that is modified by one or more amino acid modification (e.g. substitution) as described herein compared to such TACI reference sequence.

In some embodiments, the TACI polypeptide sequence is an extracellular domain region of TACI that consists essentially of only the CRD2 sequence and that is deleted in or lacks the entirety of the sequence of the CRD1 and substantially all of the stalk region. In embodiments, a TACI-Fc fusion protein with a TACI extracellular region that consists essentially only of the CRD2 with a small portion of the stalk region exhibits substantially improved cognate binding activity compared to a longer TACI molecule containing both the CRD1 and CRD2.

In particular, among TACI polypeptides provided herein is a TACI ECD sequence that has or consists of the sequence set forth in SEQ ID NO:13 (encoded by the sequence of nucleotides set forth in SEQ ID NO:48).

TACI ECD sequence (CRD2):
SEQ ID NO: 13
SLSCRKEQGKFYDHLLRDCISCASICGQHPKQCAYFCENKLRS

In some embodiments, the TACI polypeptide sequence comprises the amino acid sequence set forth in SEQ ID NO:204. In some embodiments, the TACI polypeptide sequence consists of the amino acid sequence set forth in SEQ ID NO:204. In some embodiments, the TACI polypeptide sequence comprises the amino acid sequence set forth in SEQ ID NO:206. In some embodiments, the TACI polypeptide sequence consists of the amino acid sequence set forth in SEQ ID NO:206. In some embodiments, the TACI polypeptide sequence comprises the amino acid sequence set forth in SEQ ID NO:215. In some embodiments, the TACI polypeptide sequence consists of the amino acid sequence set forth in SEQ ID NO:215. In some embodiments, the TACI polypeptide sequence comprises the amino acid sequence set forth in SEQ ID NO:217. In some embodiments, the TACI polypeptide sequence consists of the amino acid sequence set forth in SEQ ID NO:217. In some embodiments, the TACI polypeptide sequence comprises the amino acid sequence set forth in SEQ ID NO:240. In some embodiments, the TACI polypeptide sequence consists of the amino acid sequence set forth in SEQ ID NO:240. In some embodiments, the TACI polypeptide sequence comprises the amino acid sequence set forth in SEQ ID NO:241. In some embodiments, the TACI polypeptide sequence consists of the amino acid sequence set forth in SEQ ID NO: 241.

In some embodiments, the TACI polypeptide of the TACI-Fc fusion protein is a variant of any of the above TACI polypeptides in which is contained one or more amino acid substitutions (mutations) compared to such TACI polypeptide. Exemplary variant TACI polypeptides are described in Patent Publication Nos. WO2021226551 and WO 2022236335. Non-limiting examples of variant TACI polypeptides are described in the following subsection.

### a. Variant TACI Polypeptide

Provided embodiments include formulations comprising variant TACI polypeptides engineered to have improved affinity towards APRIL and/or BAFF. Among the formulations provided herein are formulations containing variant TACI polypeptides that confer improved binding affinity of the protein for BAFF and/or APRIL. In particular embodiments the provided formulations contain variant TACI polypeptides that provide for improved, combined BAFF and APRIL inhibition. In some embodiments, the improved affinity is by introduction of one or more amino acid substitutions following random mutagenesis and directed evolution of TACI extracellular domain, such as mutagenesis of residues of the second cysteine rich domain (CRD2) of TACI (e.g., spanning residues 68-110). The variant TACI polypeptides may include any TACI polypeptides described in International Patent Publication Nos. WO2021226551 and WO 2022236335, each of which are incorporated by reference in their entirety.

In some embodiments, the variant TACI polypeptides provided herein contain one or more amino acid modifications, such as one or more substitutions (alternatively, referred to herein as mutations or replacements), deletions or additions in the extracellular domain of a reference TACI polypeptide, such as a wild-type or unmodified TACI polypeptide containing a CRD(s) (hereinafter also called TDs). Thus, a provided variant TACI polypeptide is or comprises a variant TD (vTD) in which the one or more amino acid modifications (e.g. substitutions) is in a CRD. In some embodiments, the one or more amino acid modifications, such as one or more substitutions (referred to herein as mutations or replacements), deletions or additions, is in the CRD1 region. In some embodiments, the one or more amino acid modifications, such as one or more substitutions (alternatively, mutations or replacements), deletions or additions, is in the CRD2 region. In some embodiments, the one or more amino acid modifications, such as one or more substitutions (alternatively, mutations or replacements), deletions or additions, is in amino acids within both the CRD1 and CRD2 regions.

In some embodiments, the reference (e.g. unmodified) TACI sequence is a wild-type TACI sequence or is a portion thereof that contains one or both CRDs. In some embodiments, the reference (e.g., unmodified) TACI is or comprises the extracellular domain (ECD) of TACI or a portion thereof containing one or both CRD domains. In some embodiments, the extracellular domain of a reference (e.g., unmodified) TACI polypeptide comprises a CRD1 and CRD2. However, the variant TACI polypeptide need not comprise both the CRD1 and the CRD2. In some embodiments, the variant TACI polypeptide comprises or consists essentially of the CRD1 or a specific binding fragment thereof. In some embodiments, the variant TACI polypeptide comprises or consists essentially of the CRD2 or specific binding fragments thereof. In some embodiments, the variant TACI is a soluble polypeptide and lacks a transmembrane domain.

In some embodiments, the reference (e.g., unmodified) TACI sequence is a mammalian TACI sequence. In some embodiments, the reference (e.g., unmodified) TACI sequence can be a mammalian TACI that includes, but is not limited to, human, mouse, cynomolgus monkey, or rat. In some embodiments, the reference (e.g., unmodified) TACI sequence is human. The extracellular domain of an exemplary human TACI sequence is set forth in SEQ ID NO:122.

In some embodiments, the reference (e.g., unmodified) TACI sequence has (i) the sequence of amino acids set forth in SEQ ID NO:122 or a sequence thereof that lacks the N-terminal methionine, (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:122 and that binds to APRIL, BAFF or an APRIL/BAFF heterotrimer, or (iii) is a fragment or portion of (i) or (ii) containing a CRD1 and/or CRD2, in which the portion binds to APRIL, BAFF or an APRIL/BAFF heterotrimer. In some embodiments, the reference (e.g., unmodified) TACI sequence lacks the N-terminal methionine as set forth in SEQ ID NO: 122.

In some embodiments, the reference (e.g. unmodified) TACI sequence is an extracellular domain sequence of TACI that is a portion of the ECD that contains an N-terminal deletion relative to the sequence of amino acids set forth in SEQ ID NO:122. In some embodiments, the N-terminal deletion is deletion of N-terminal amino acid residues 1-28 corresponding to residues set forth in SEQ ID NO:122. In some embodiments, the N-terminal deletion is deletion of N-terminal amino acid residues 1-29 corresponding to residues set forth in SEQ ID NO:122. In some embodiments, the N-terminal deletion is deletion of N-terminal amino acid residues 1-30 corresponding to residues set forth in SEQ ID NO:122. In some embodiments, the N-terminal deletion is deletion of N-terminal amino acid residues 1-31 corresponding to residues set forth in SEQ ID NO:122. In some embodiments, the N-terminal deletion is deletion of N-terminal amino acid residues 1-32 corresponding to residues set forth in SEQ ID NO:122. In some embodiments, the N-terminal deletion is deletion of N-terminal amino acid residues 1-33 corresponding to residues set forth in SEQ ID NO:122.

In embodiments of any of the provided embodiments, the reference (e.g. unmodified) TACI sequence is an ECD portion that contains deletion of one or more residues of the stalk portion of the TACI extracellular domain. In some embodiments, the reference (e.g. unmodified) TACI sequence is an ECD portion that lacks one or more contiguous C-terminal amino acid residues beginning at residue 105 and up to or including amino acid residue 166 corresponding to residues of the ECD sequence set forth in SEQ ID NO:122. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61 or 62 residues of the ECD sequence are deleted.

In some embodiments, the reference (e.g. unmodified) TACI sequence contains an ECD portion having a contiguous sequence of amino acids that includes the CRD1 and/or CRD2 (e.g. CRD1 and CRD2 or CRD2 only) and only a segment or portion of the stalk sequence. Suitable stalk segments include one or more amino acids of amino acid residues 105 to 154 of SEQ ID NO:122. For example, the stalk segment can consist of the following with reference to SEQ ID NO:122: amino acid residue 105, amino acid residues 105 to 106, amino acid residues 105 to 107, amino acid residues 105 to 108, amino acid residues 105 to 109, amino acid residues 105 to 110, amino acid residues 105 to 111, amino acid residues 105 to 112, amino acid residues 105 to 113, amino acid residues 105 to 114, amino acid residues 105 to 115, amino acid residues 105 to 116, amino acid residues 105 to 117, amino acid residues 105 to 118, amino acid residues 105 to 119, amino acid residues 105 to 120, amino acid residues 105 to 121, amino acid residues 105 to 122, amino acid residues 105 to 123, amino acid residues 105 to 124, amino acid residues 105 to 125, amino acid residues 105 to 126, amino acid residues 105 to 127, amino acid residues 105 to 128, amino acid residues 105 to 129, amino acid residues 105 to 130, amino acid residues 105 to 131, amino acid residues 105 to 132, amino acid residues 105 to 133, amino acid residues 105 to 134, amino acid residues 105 to 135, amino acid residues 105 to 136, amino acid residues 105 to 137, amino acid residues 105 to 138, amino acid residues 105 to 139, amino acid residues 105 to 140, amino acid residues 105 to 141, amino acid residues 105 to 142, amino acid residues 105 to 143, amino acid residues 105 to 144, amino acid residues 105 to 145, amino acid residues 105 to 146, amino acid residues 105 to 147, amino acid residues 105 to 148, amino acid residues 105 to 149, amino acid residues 105 to 150, amino acid residues 105 to 151, amino acid residues 105 to 152, amino acid residues 105 to 153, and amino acid residues 105 to 154.

In some embodiments, the reference (e.g. unmodified) TACI sequence lacks or is mutated in one or more potential furin cleavage sites. In some cases, the reference (e.g. unmodified) TACI sequence is an ECD or portion that in which the arginine residue at position 119 is mutated, e.g. R119G. In some cases, the reference (e.g. unmodified) TACI sequence is an ECD or portion that in which the glutamine residue at position 121 is mutated, e.g. Q121P. In some cases, the reference (e.g. unmodified) TACI sequence is an ECD or portion that in which the arginine residue at position 122 is mutated, e.g. R122Q.

In some embodiments, the reference TACI sequence is a TACI ECD sequence as set forth in international PCT publication No. WO2000/067034, WO2002/094852 or WO2008/154814.

In some embodiments, the reference TACI sequence is a TACI ECD sequence that has or consists of the sequence set forth in SEQ ID NO:131.

In some embodiments, the reference TACI sequence is a TACI ECD sequence that has or consists of the sequence set forth in SEQ ID NO:130.

In some embodiments, the reference TACI sequence is a TACI ECD sequence that has or consists of the sequence set forth in SEQ ID NO:1 (encoded by the sequence of nucleotides set forth in SEQ ID NO:36).

In some embodiments, the reference TACI sequence is an extracellular domain region of TACI that consists essentially of only the CRD2 sequence and that is deleted in or lacks the entirety of the sequence of the CRD1 and substantially all of the stalk region. In some embodiments, the reference TACI sequence has or consists of the sequence set forth in SEQ ID NO:13, in which a provided variant TACI polypeptide is modified by one or more amino acid modification (e.g. substitution) as described herein compared to such reference TACI sequence.

In some embodiments, the reference TACI sequence comprises the amino acid sequence set forth in SEQ ID NO:204. In some embodiments, the reference TACI sequence consists of the amino acid sequence set forth in SEQ ID NO:204. In some embodiments, the reference TACI sequence comprises the amino acid sequence set forth in SEQ ID NO:206. In some embodiments, the reference TACI sequence consists of the amino acid sequence set forth in SEQ ID NO:206. In some embodiments, the reference TACI sequence comprises the amino acid sequence encoded by the nucleic acid sequence set forth in SEQ ID NO:215. In some embodiments, the reference TACI sequence consists of the amino acid sequence encoded by the nucleic acid sequence set forth in SEQ ID NO:215. In some embodiments, the reference TACI sequence comprises the amino acid sequence encoded by the nucleic acid sequence set forth in SEQ ID NO:217. In some embodiments, the reference TACI sequence consists of the amino acid sequence encoded by the nucleic acid sequence set forth in SEQ ID NO:217. In some embodiments, the reference TACI sequence comprises the amino acid sequence set forth in SEQ ID NO:240. In some embodiments, the reference TACI sequence consists of the amino acid sequence set forth in SEQ ID NO:240. In some embodiments, the reference TACI sequence comprises the amino acid sequence set forth in SEQ ID NO:241. In some embodiments, the reference TACI sequence consists of the amino acid sequence set forth in SEQ ID NO: 241.

In any of the provided embodiments, the variant TACI sequence has the sequence of the reference (e.g. unmodified) TACI sequence, such as any described above, but additionally contains one more amino acid modifications, such as one or more amino acid substitutions. In particular, provided herein are variant TACI polypeptides containing at least one affinity-modified TD domain (e.g., CRD1 and/or CRD2) or a specific binding fragment thereof that contains one or more amino acid substitutions in a TD domain of a reference (e.g., unmodified or wild-type) TACI polypeptide, such that the variant TACI polypeptide exhibits altered (e.g. increased) binding activity or affinity for one or both of APRIL or BAFF compared to the reference (e.g., unmodified or wild-type) TACI polypeptide. In some embodiments, a variant TACI polypeptide has a binding affinity for APRIL and/or BAFF that differs from that of a reference (e.g., unmodified or wild-type) TACI polypeptide control sequence as determined by, for example, solid-phase ELISA immunoassays, flow cytometry or Biacore assays. Binding affinities for each of the cognate binding partners are independent; that is, in some embodiments, a variant TACI polypeptide has an increased binding affinity for one or both APRIL and BAFF, and a decreased or unchanged binding affinity for the other of APRIL or BAFF, relative to a reference (e.g., unmodified or wild-type) TACI polypeptide.

In some embodiments, the variant TACI polypeptide has an increased binding affinity for BAFF, relative to the reference (unmodified or wild-type) TACI polypeptide. In some embodiments, the variant TACI polypeptide has an increased binding affinity for APRIL relative to the reference (unmodified or wild-type) TACI polypeptide. In some embodiments, the variant TACI polypeptide has an increased binding affinity for APRIL and BAFF relative to the reference (unmodified or wild-type) TACI polypeptide. The cognate ligands BAFF and/or APRIL can be a mammalian protein, such as a human protein or a murine protein. In particular embodiments, the cognate ligands BAFF and/or APRIL are human. In some embodiments, a variant TACI polypeptide with increased or greater binding affinity to APRIL and/or BAFF will have an increase in binding affinity relative to the reference (e.g., unmodified or wild-type) TACI polypeptide control of at least about 5%, such as at least about 10%, 15%, 20%, 25%, 35%, or 50%. In some embodiments, the increase in binding affinity relative to the reference (e.g., unmodified or wild-type) TACI polypeptide is more than about 1.2-fold, about 1.5-fold, about 2-fold, about 3-fold, about 4-fold, about 5-fold, about 6-fold, about 7-fold, about 8-fold, about 9-fold, about 10-fold, about 20-fold, about 30-fold, about 40-fold or about 50-fold. In any of the examples, the reference (e.g., unmodified or wild-type) TACI polypeptide has the same sequence as the variant TACI polypeptide except that it does not contain the one or more amino acid modifications (e.g., substitutions).

In some embodiments, the equilibrium dissociation constant (K_{d}) of any of the foregoing embodiments to BAFF can be less than 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹M, or 1×10⁻¹² M. In some embodiments, the K_{d} of any of the foregoing embodiments to BAFF is less than at or about 1×10⁻⁹ M, 1×10⁻¹⁰ M or 1×10⁻¹¹M, or 1×10⁻¹² M. In some embodiments, the K_{d} of any of the foregoing embodiments to BAFF is between 1×10⁻⁹ M and at or about 1×10⁻¹² M. In some embodiments, the K_{d} of any of the foregoing embodiments to BAFF is at or about 1×10⁻⁹ M, at or about 2×10⁻⁹ M, at or about 4×10⁻⁹ M, at or about 6×10⁻⁹ M, at or about 8×10⁻⁹ M, at or about 1×10⁻¹⁰ M, at or about 2×10⁻¹⁰ M, at or about 4×10⁻¹⁰ M, at or about 6×10⁻¹⁰ M, at or about 8×10⁻¹⁰ M, at or about 1×10⁻¹¹ M, at or about 2×10⁻¹¹ M, at or about 4×10⁻¹¹ M, at or about 6×10⁻¹¹ M, at or about 8×10⁻¹¹ M, or at or about 1×10⁻¹²M, or any value between any of the foregoing. In some embodiments, a provided embodiment includes a variant TACI polypeptide as described above and the K_{d} to BAFF is decreased (higher binding affinity) by greater than or greater than about 1.5-fold, such as greater than or about 2-fold, 3-fold, 4-fold, 5-fold, 6-fold, 7-fold, 8-fold, 9-fold, 10-fold or more.

In some embodiments, the equilibrium dissociation constant (K_{d}) of any of the foregoing embodiments to APRIL can be less than 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹M, or 1×10⁻² M. In some embodiments, the K_{d} of any of the foregoing embodiments to APRIL is less than at or about 1×10⁻⁹ M, 1×10⁻¹⁰ M or 1×10⁻¹¹M, or 1×10⁻¹² M. In some embodiments, the K_{d} of any of the foregoing embodiments to APRIL is between 1×10⁻⁹ M and at or about 1×10⁻¹² M. In some embodiments, the K_{d} of any of the foregoing embodiments to APRIL is at or about 1×10⁻⁹ M, at or about 2×10⁻⁹ M, at or about 4×10⁻⁹ M, at or about 6×10⁻⁹ M, at or about 8×10⁻⁹ M, at or about 1×10⁻¹⁰ M, at or about 2×10⁻¹⁰ M, at or about 4×10⁻¹⁰ M, at or about 6×10⁻¹⁰ M, at or about 8×10⁻¹⁰ M, at or about 1×10⁻¹¹ M, at or about 2×10⁻¹¹ M, at or about 4×10⁻¹¹ M, at or about 6×10⁻¹¹ M, at or about 8×10⁻¹¹ M, or at or about 1×10⁻¹²M, or any value between any of the foregoing. In some embodiments, a provided embodiment includes a variant TACI polypeptide as described above and the K_{d} to APRIL is decreased (higher binding affinity) by greater than or greater than about 1.5-fold, such as greater than or about 2-fold, 3-fold, 4-fold, 5-fold, 6-fold, 7-fold, 8-fold, 9-fold, 10-fold or more.

The reference (e.g., unmodified or wild-type) TACI sequence does not necessarily have to be used as a starting composition to generate variant TACI polypeptides described herein. Therefore, use of the term “modification”, such as “substitution” does not imply that the present embodiments are limited to a particular method of making variant TACI polypeptides or immunomodulatory proteins containing the same. Variant TACI polypeptides can be made, for example, by de novo peptide synthesis and thus does not necessarily require a modification, such as a “substitution”, in the sense of altering a codon to encode for the modification, e.g. substitution. This principle also extends to the terms “addition” and “deletion” of an amino acid residue which likewise do not imply a particular method of making. The means by which the variant TACI polypeptides are designed or created is not limited to any particular method. In some embodiments, however, a reference (e.g., unmodified or wild-type) TACI encoding nucleic acid is mutagenized from reference (e.g., unmodified or wild-type) TACI genetic material and screened for desired specific binding affinity or other functional activity. In some embodiments, a variant TACI polypeptide is synthesized de novo utilizing protein or nucleic acid sequences available at any number of publicly available databases and then subsequently screened. The National Center for Biotechnology Information provides such information, and its website is publicly accessible via the internet as is the UniProtKB database as discussed previously.

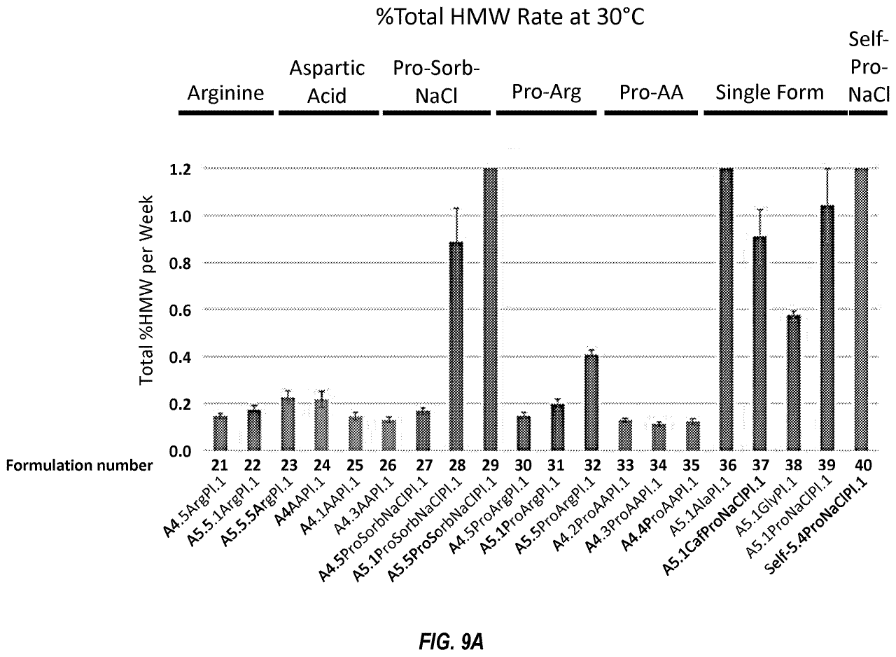

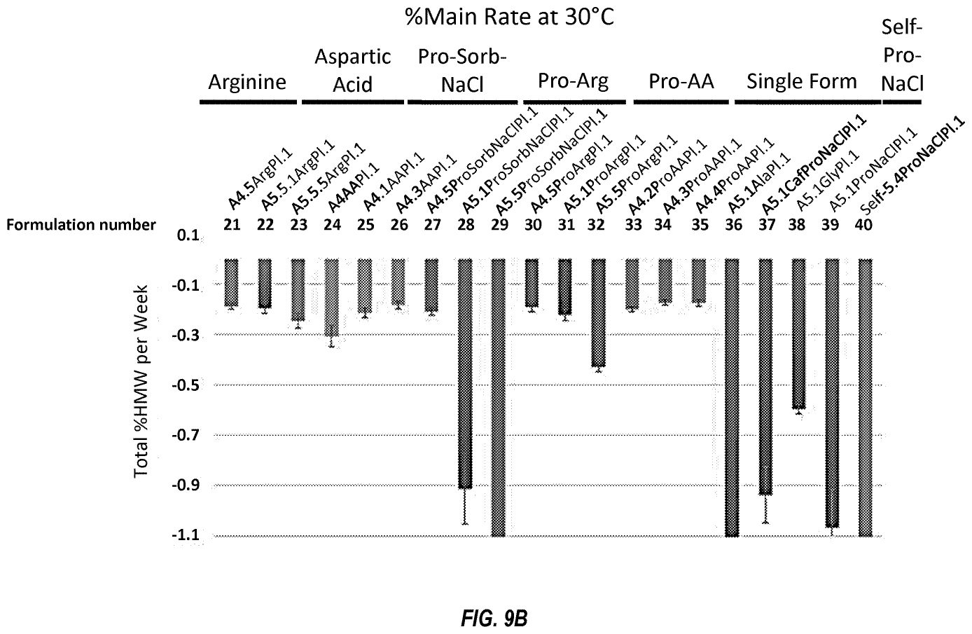

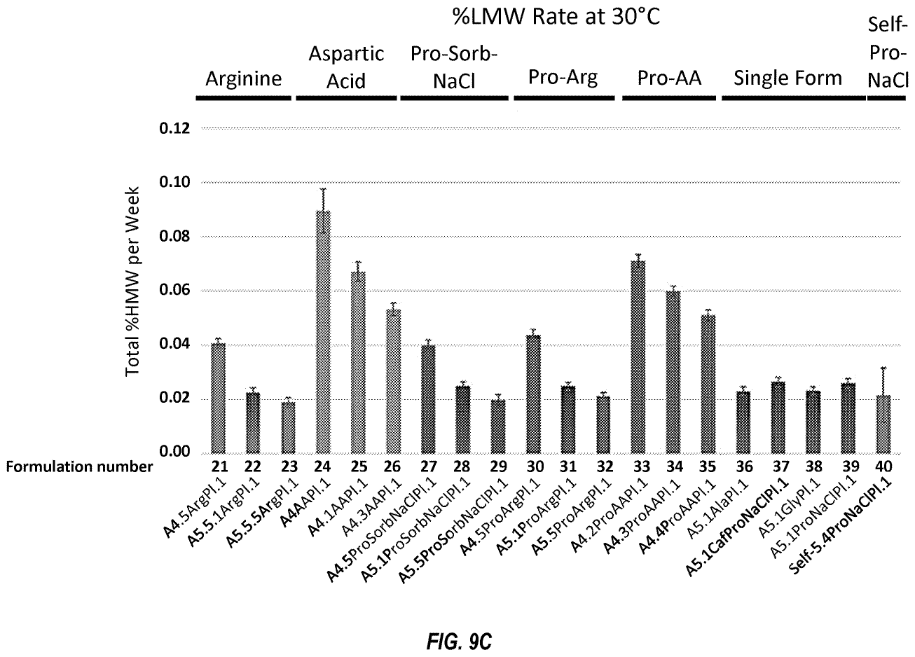

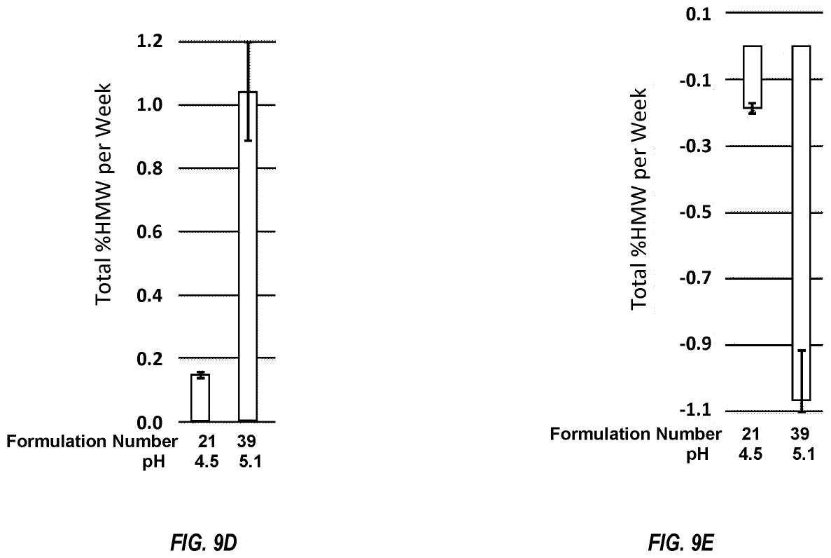

Unless stated otherwise, as indicated throughout the present disclosure, the amino acid modification(s) in a variant TACI polypeptide are designated by amino acid position number corresponding to the numbering of positions of the reference ECD sequence set forth in SEQ ID NO:122. It is within the level of a skilled artisan to identify the corresponding position of a modification, e.g. amino acid substitution, in a TACI polypeptide, including portion thereof containing TD (e.g. CRD1 and/or CRD2) thereof, such as by alignment of a reference sequence (e.g. SEQ ID NO:1 or 13) with SEQ ID NO:122. An alignment identifying corresponding residues is exemplified in FIG. 9. In the listing of modifications throughout this disclosure, the amino acid position is indicated in the middle, with the corresponding reference (e.g. unmodified or wild-type) amino acid listed before the number and the identified variant amino acid substitution listed after the number. If the modification is a deletion of the position a “del” is indicated and if the modification is an insertion at the position an “ins” is indicated. In some cases, an insertion is listed with the amino acid position indicated in the middle, with the corresponding reference amino acid listed before and after the number and the identified variant amino acid insertion listed after the unmodified (e.g. wild-type) amino acid.

In some embodiments, the variant TACI polypeptide has one or more amino acid modification, e.g. substitution relative to a reference (e.g., unmodified or wild-type) TACI sequence, such as any as described. The one or more amino acid modification, e.g. substitution, can be in the ectodomain (extracellular domain) of the reference (e.g., unmodified or wild-type) TACI sequence. In some embodiments, the one or more amino acid modification, e.g. substitution is in the CRD1 domain or specific binding fragment thereof. In some embodiments, the one or more amino acid modification, e.g. substitution is in the CRD2 domain or specific binding fragment thereof. In some embodiments of the variant TACI polypeptide, some of the one or more amino acid modification, e.g. substitution is in the CRD1 domain or a specific binding fragment thereof, and some of the one or more amino acid modification, e.g. substitution is in the CRD2 domain or a specific binding fragment thereof.

In some embodiments, the variant TACI polypeptide has up to 1, 2, 3, 4, 5, 6, 7, 8, 9, 10, 11, 12, 13, 14, 15, 16, 17, 18, 19, or 20 amino acid modification(s), e.g. substitution, in the reference TACI sequence. The modification, e.g. substitution can be in the CRD1 domain or the CRD2 domain. In some embodiments, the variant TACI polypeptide has up to 1, 2, 3, 4, 5, 6, 7, 8, 9, 10, 11, 12, 13, 14, 15, 16, 17, 18, 19, or 20 amino acid substitutions in the CRD1 domain or specific binding fragment thereof of the reference TACI sequence. In some embodiments, the variant TACI polypeptide has up to 1, 2, 3, 4, 5, 6, 7, 8, 9, 10, 11, 12, 13, 14, 15, 16, 17, 18, 19, or 20 amino acid substitutions in the CRD2 domain or specific binding fragment thereof of the reference TACI sequence.

In some embodiments, the variant TACI polypeptide containing the one or more amino acid modifications (e.g. amino acid substations) as described has at least about 85%, 86%, 86%, 88%, 89%, 90%, 91%, 92%, 93%, 94%, 95%, 96%, 97%, 98%, or 99% sequence identity with the reference (e.g., unmodified or wild-type) TACI polypeptide set forth in SEQ ID NO: 122 or specific binding fragment thereof containing the CRD1 and/or CRD2 domain. In some embodiments, the specific binding fragment contains the CRD1 domain, e.g. the specific binding fragment contains the sequence set forth as amino acids 34-66 of SEQ ID NO:122. In some cases, the CRD1 domain is the only full CRD domain in the specific binding fragment. In some embodiments, the specific binding fragment is or contains the CRD2 domain, e.g. the specific binding fragment contains the sequence set forth as amino acids 71-104 of SEQ ID NO:122. In some cases, the CRD2 domain is the only full CRD domain in the specific binding fragment. In some embodiments, the specific binding fragment is or contains the CRD1 domain and the CRD2 domain, e.g. the specific binding fragment contains amino acids 34-104 of SEQ ID NO:122. In some embodiments, the specific binding fragment contains a contiguous portion of the stalk domain, e.g. the specific binding fragment contains a contiguous portion of amino acids 105-165 of SEQ ID NO:122. In embodiments of any embodiments, the specific binding fragment of SEQ ID NO:122 is less than the full-length ECD set forth in SEQ ID NO:122. In some embodiments, the specific binding fragment is set forth in SEQ ID NO: 1. In some embodiments, the specific binding fragment is set forth in SEQ ID NO:13. In some embodiments, the specific binding fragment is set forth in SEQ ID NO: 130. In some embodiments, the specific binding fragment is set forth in SEQ ID NO:131.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the reference (e.g., unmodified or wild-type) TACI polypeptide or specific binding fragment thereof, such as with the amino acid sequence of SEQ ID NO: 1, 13 or 122.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 122.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 1.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 13.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 130.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 131.

In some embodiments, the variant TACI polypeptide has one or more amino acid modification, e.g. substitution relative to a reference TACI polypeptide or specific binding fragment there of corresponding to position(s) 40, 59, 60, 61, 74, 75, 76, 77, 78, 79, 82, 83, 84, 85, 86, 87, 88, 92, 95, 97, 98, 99, 101, 102 and 103 with reference to numbering of SEQ ID NO:122. In some embodiments, the variant TACI polypeptide has one or more amino acid modification, e.g. substitution selected from W40R, Q59R, R60G, T61P, E74V, Q75E, Q75R, G76S, K77E, F78Y, Y79F, L82H, L82P, L83S, R84G, R84L, R84Q, D85E, D85V, C86Y, I87L, I87M, S88N, I92V, Q95R, P97S, K98T, Q99E, A101D, Y102D, F103S, F103V, F103Y, or a conservative amino acid substitution thereof. In some embodiments, the reference TACI polypeptide includes the CRD1 domain or CRD2 domain, for example the reference TACI polypeptide is set forth in SEQ ID NO: 1 or SEQ ID NO:122.

In some embodiments, the amino acid substitutions are in the CRD2 domain only. In some embodiments, the variant TACI polypeptide has one or more amino acid modification, e.g. substitution relative to a reference TACI polypeptide or specific binding fragment thereof corresponding to position(s) 74, 75, 76, 77, 78, 79, 82, 83, 84, 85, 86, 87, 88, 92, 95, 97, 98, 99, 101, 102 and 103 with reference to numbering of SEQ ID NO:122. In some embodiments, the variant TACI polypeptide has one or more amino acid modification, e.g. substitution selected from E74V, Q75E, Q75R, G76S, K77E, F78Y, Y79F, L82H, L82P, L83S, R84G, R84L, R84Q, D85E, D85V, C86Y, I87L, I87M, S88N, I92V, Q95R, P97S, K98T, Q99E, A101D, Y102D, F103S, F103V, F103Y, or a conservative amino acid substitution thereof. In some embodiments, among the CRD domains, the reference TACI polypeptide includes only the CRD2 domain but lacks the CRD1 domain, for example the reference TACI polypeptide is set forth in SEQ ID NO: 13. Accordingly, in some embodiments, the variant TACI polypeptide includes a portion of the ECD sequence of a TACI polypeptide that includes the CRD2 domain but lacks the CRD1 domain.

A conservative amino acid modification, e.g. substitution is any amino acid that falls in the same class of amino acids as the substituted amino acids, other than the reference (e.g., unmodified) or wild-type amino acid. The classes of amino acids are aliphatic (glycine, alanine, valine, leucine, and isoleucine), hydroxyl or sulfur-containing (serine, cysteine, threonine, and methionine), cyclic (proline), aromatic (phenylalanine, tyrosine, tryptophan), basic (histidine, lysine, and arginine), and acidic/amide (aspartate, glutamate, asparagine, and glutamine).

In some embodiments, the variant TACI polypeptide includes at least one amino acid substitution at position 75 with reference to numbering of SEQ ID NO:122. In some embodiments, the amino acid substitution at position 75 confers increased binding to BAFF or APRIL compared to the reference (e.g. wildtype or unmodified) TACI polypeptide not containing the amino acid substitution. In some embodiments, the substituted amino acid is an acidic amino acid or amide, such as to a different acidic amino acid or amide compared to the reference (e.g. wildtype or unmodified) TACI polypeptide. In some embodiments, the substituted amino acid at position 75 is a glutamic acid (Glu, E). In some embodiments, the substituted amino acid at position 75 is an aspartic acid (Asp, D). In some embodiments, the substituted amino acid at position 75 is an asparagine (Asn, N). In some embodiments, the substituted amino acid at position 75 is a glutamine (Gln, Q).

In some embodiments, the variant TACI polypeptide includes at least one amino acid substitution at position 77 with reference to numbering of SEQ ID NO:122. In some embodiments, the amino acid substitution at position 77 confers increased binding to BAFF or APRIL compared to the reference (e.g. wildtype or unmodified) TACI polypeptide not containing the amino acid substitution. In some embodiments, the substituted amino acid at position 77 is an acidic amino acid or amide. In some embodiments, the substituted amino acid at position 77 is a glutamic acid (Glu, E). In some embodiments, the substituted amino acid at position 77 is an aspartic acid (Asp, D). In some embodiments, the substituted amino acid at position 77 is an asparagine (Asn, N). In some embodiments, the substituted amino acid at position 77 is a glutamine (Gln, Q).

In some embodiments, the variant TACI polypeptide includes at least one amino acid substitution at position 78 with reference to numbering of SEQ ID NO:122. In some embodiments, the amino acid substitution at position 78 confers increased binding to BAFF or APRIL compared to the reference (e.g. wildtype or unmodified) TACI polypeptide not containing the amino acid substitution. In some embodiments, the substituted amino acid at position 78 is an aromatic amino acid, such as to a different aromatic amino acid compared to the reference (e.g. wildtype or unmodified) TACI polypeptide. In some embodiments, the substituted amino acid at position 78 is a phenylalanine (Phe, F). In some embodiments, the substituted amino acid at position 78 is a tyrosine (Tyr, Y). In some embodiments, the substituted amino acid at position 78 is a tryptophan (Trp, W).

In some embodiments, the variant TACI polypeptide includes at least one amino acid substitution at position 84 with reference to numbering of SEQ ID NO:122. In some embodiments, the amino acid substitution at position 84 confers increased binding to BAFF or APRIL compared to the reference (e.g. wildtype or unmodified) TACI polypeptide not containing the amino acid substitution. In some embodiments, the substituted amino acid at position 84 is an acidic amino acid or amide. In some embodiments, the substituted amino acid at position 84 is a glutamic acid (Glu, E). In some embodiments, the substituted amino acid at position 84 is an aspartic acid (Asp, D). In some embodiments, the substituted amino acid at position 84 is an asparagine (Asn, N). In some embodiments, the substituted amino acid at position 84 is a glutamine (Gln, Q).

In some embodiments, the variant TACI polypeptide includes at least one amino acid substitution at position 101 with reference to numbering of SEQ ID NO:122. In some embodiments, the amino acid substitution at position 101 confers increased binding to BAFF or APRIL compared to the reference (e.g. wildtype or unmodified) TACI polypeptide not containing the amino acid substitution. In some embodiments, the substituted amino acid at position 101 is an acidic amino acid or amide. In some embodiments, the substituted amino acid at position 101 is a glutamic acid (Glu, E). In some embodiments, the substituted amino acid at position 101 is an aspartic acid (Asp, D). In some embodiments, the substituted amino acid at position 101 is an asparagine (Asn, N). In some embodiments, the substituted amino acid at position 101 is a glutamine (Gln, Q).

In some embodiments, the variant TACI polypeptide includes at least one amino acid substitution at position 102 with reference to numbering of SEQ ID NO:122. In some embodiments, the amino acid substitution at position 102 confers increased binding to BAFF or APRIL compared to the reference (e.g. wildtype or unmodified) TACI polypeptide not containing the amino acid substitution. In some embodiments, the substituted amino acid at position 102 is an acidic amino acid or amide. In some embodiments, the substituted amino acid at position 102 is a glutamic acid (Glu, E). In some embodiments, the substituted amino acid at position 102 is an aspartic acid (Asp, D). In some embodiments, the substituted amino acid at position 102 is an asparagine (Asn, N). In some embodiments, the substituted amino acid at position 102 is a glutamine (Gln, Q).

In some embodiments, the variant TACI polypeptide includes at least one amino acid substitution E74V. In some embodiments, the variant TACI polypeptide includes at least one amino acid substitution Q75E. In some embodiments, the variant TACI polypeptide includes at least one amino acid substitution K77E. In some embodiments, the variant TACI polypeptide includes at least one amino acid substitution F78Y. In some embodiments, the variant TACI polypeptide includes at least one amino acid substitution Y79F. In some embodiments, the variant TACI polypeptide includes at least one amino acid substitution L82H. In some embodiments, the variant TACI polypeptide includes at least one amino acid substitution L82P. In some embodiments, the variant TACI polypeptide includes at least one amino acid substitution R84G. In some embodiments, the variant TACI polypeptide includes at least one amino acid substitution R84L. In some embodiments, the variant TACI polypeptide includes at least one amino acid substitution R84Q. In some embodiments, the variant TACI polypeptide includes at least one amino acid substitution D85V. In some embodiments, the variant TACI polypeptide includes at least one amino acid substitution C86Y. In some embodiments, the variant TACI polypeptide includes at least one amino acid substitution A101D. In some embodiments, the variant TACI polypeptide includes at least one amino acid substitution Y102D. In some embodiments, the variant TACI polypeptide contains two or more amino acid substitutions of any two or more of the foregoing. In some embodiments, the variant TACI polypeptide includes one or more amino acid substitution that is a conservative amino acid substitution of any of the foregoing. In provided embodiments, the variant TACI polypeptide includes the at least one amino acid substitution in any reference TACI polypeptide sequence as described. In some embodiments, the at least one amino acid substitution is in the reference TACI sequence set forth in SEQ ID NO: 1. In some embodiments, the at least one amino acid substitution is in the reference TACI sequence set forth in SEQ ID NO: 13. In some embodiments, the at least one amino acid substitution is in the reference TACI sequence set forth in SEQ ID NO: 130. In some embodiments, the at least one amino acid substitution is in the reference TACI sequence set forth in SEQ ID NO: 131.

In some embodiments, the variant TACI polypeptide includes the amino acid substitution E74V. In some embodiments, the variant TACI polypeptide includes the amino acid substitution Q75E. In some embodiments, the variant TACI polypeptide includes the amino acid substitution K77E. In some embodiments, the variant TACI polypeptide includes the amino acid substitution F78Y. In some embodiments, the variant TACI polypeptide includes the amino acid substitution Y79F. In some embodiments, the variant TACI polypeptide includes the amino acid substitution L82H. In some embodiments, the variant TACI polypeptide includes the amino acid substitution L82P. In some embodiments, the variant TACI polypeptide includes the amino acid substitution R84G. In some embodiments, the variant TACI polypeptide includes the amino acid substitution R84L. In some embodiments, the variant TACI polypeptide includes the amino acid substitution R84Q. In some embodiments, the variant TACI polypeptide includes the amino acid substitution D85V. In some embodiments, the variant TACI polypeptide includes the amino acid substitution C86Y. In some embodiments, the variant TACI polypeptide includes the amino acid substitution A102D. In some embodiments, the variant TACI polypeptide includes the amino acid substitution Y102D. In some embodiments, the variant TACI polypeptide contains two or more amino acid substitutions of any two or more of the foregoing. In some embodiments, the variant TACI polypeptide includes one or more of amino acid substitution that is a conservative amino acid substitution of any of the foregoing. In provided embodiments, the variant TACI polypeptide includes the amino acid substitution in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the amino acid substitutions are D85E/K98T. In some embodiments, the amino acid substitutions are I87L/K98T. In some embodiments, the amino acid substitutions are R60G/Q75E/L82P. In some embodiments, the amino acid substitutions are R60G/C86Y. In some embodiments, the amino acid substitutions are W40R/L82P/F103Y. In some embodiments, the amino acid substitutions are W40R/Q59R/T61P/K98T. In some embodiments, the amino acid substitutions are L82P/I87L. In some embodiments, the amino acid substitutions are G76S/P97S. In some embodiments, the amino acid substitutions are K77E/R84L/F103Y. In some embodiments, the amino acid substitutions are Y79F/Q99E. In some embodiments, the amino acid substitutions are L83S/F103S. In some embodiments, the amino acid substitutions are K77E/R84Q. In some embodiments, the amino acid substitutions are K77E/A101D. In some embodiments, the amino acid substitutions are K77E/F78Y/Y102D. In some embodiments, the amino acid substitutions are Q75E/R84Q. In some embodiments, the amino acid substitutions are Q75R/R84G/I92V. In some embodiments, the amino acid substitutions are K77E/A101D/Y102D. In some embodiments, the amino acid substitutions are R84Q/S88N/A101D. In some embodiments, the amino acid substitutions are R84Q/F103V. In some embodiments, the amino acid substitutions are K77E/Q95R/A101D. In some embodiments, the amino acid substitutions are I87M/A101D. In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In embodiments of any embodiments, the variant TACI polypeptide includes one or more amino acid substitutions from Q75E, K77E, F78Y, R84G, R84Q, A101D or Y102D, or any combination thereof. In some embodiments, the variant TACI polypeptide includes any 1, 2, 3, 4, 5 or 6 of the above amino acid substitutions. In some embodiments, the variant TACI polypeptide contains one of the above amino acid substitutions. In some embodiments, the variant TACI polypeptide contains two of the above amino acid substitutions. In some embodiments, the variant TACI polypeptide contains three of the above amino acid substitutions. In some embodiments, the variant TACI polypeptide contains four of the above amino acid substitutions. In some embodiments, the variant TACI polypeptide contains five of the above amino acid substitutions. In some embodiments, the variant TACI polypeptide contains six of the above amino acid substitutions.

In embodiments of any embodiments, the one or more amino acid substitutions comprise Q75E/R84Q. In embodiments of any embodiments, the one or more amino acid substitutions comprise Q75E/K77E. In embodiments of any embodiments, the one or more amino acid substitutions comprise Q75E/F78Y. In embodiments of any embodiments, the one or more amino acid substitutions comprise Q75E/A101D. In embodiments of any embodiments, the one or more amino acid substitutions comprise Q75E/Y102D. In embodiments of any embodiments, the one or more amino acid substitutions comprise K77E/F78Y. In embodiments of any embodiments, the one or more amino acid substitutions comprise K77E/R84Q. In embodiments of any embodiments, the one or more amino acid substitutions comprise K77E/A101D. In embodiments of any embodiments, the one more amino acid substitutions comprise K77E/Y102D. In embodiments of any embodiments, the one or more amino acid substitutions comprise F78Y/R84Q. In embodiments of any embodiments, the one or more amino acid substitutions comprise F78Y/A101D. In embodiments of any embodiments, the one or more amino acid substitutions comprise F78Y/Y102D. In embodiments of any embodiments, the one or more amino acid substitutions comprise R84Q/A101D. In embodiments of any embodiments, the one or more amino acid substitutions comprise R84Q/Y102D. In embodiments of any embodiments, the one or more amino acid substitutions comprise A101D/Y102D. In provided embodiments, the variant TACI polypeptide includes the amino acid substitutions in any reference TACI polypeptide sequence as described, such as in the sequence set forth in SEQ ID NO:1, SEQ ID NO:13, SEQ ID NO:130 or SEQ ID NO: 131.

In some embodiments, the variant TACI polypeptides include the amino acid substitution(s) R84G, A101D, K77E/R84Q, K77E/A101D, K77E/F78Y, K77E/F78Y/Y102D, Q75E/R84Q, K77E/A101D/Y102D, R84Q, K77E, A101D, Q75E, K77E/F78Y/R84Q, F78Y, F78Y/R84Q, F78Y/A101D, F78Y/Y102D, or K77E/Y102D. In provided embodiments, the variant TACI polypeptide includes the amino acid substitutions in any reference TACI polypeptide sequence as described, such as in the sequence set forth in SEQ ID NO:1, SEQ ID NO:13, SEQ ID NO:130 or SEQ ID NO: 131.

In some embodiments, the variant TACI polypeptide includes the amino acid substitutions K77E and F78Y (K77E/F78Y). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide includes the amino acid substitutions K77E and Y102D (K77E/Y102D). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide contains the amino acid substitutions F78Y and Y102D (F78Y/Y012D). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments the variant TACI polypeptide contains the amino acid substitutions K77E, F78Y and Y102D (K77E/F78Y/Y102D). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide contains the amino acid substitutions Q75E/R84Q. In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide comprises any of the mutations listed in Table 1. Table 1 also provides exemplary sequences by reference to the SEQ ID NO of the reference (e.g., unmodified) TACI polypeptide, and exemplary variant TACI polypeptides. As indicated, the exact locus or residues corresponding to a given domain can vary, such as depending on the methods used to identify or classify the domain. Also, in some cases, adjacent N- and/or C-terminal amino acids of a given domain (e.g. CRD) also can be included in a sequence of a variant TACI polypeptide, such as to ensure proper folding of the domain when expressed. Thus, it is understood that the exemplification of the SEQ ID NOs in Table 1 is not to be construed as limiting. For example, the particular domain, such as the ECD domain or a portion thereof containing the CRD1/CRD2 or CRD2 only, of a variant TACI polypeptide can be several amino acids longer or shorter, such as 1-10, e.g., 1, 2, 3, 4, 5, 6 or 7 amino acids longer or shorter, than the sequence of amino acids set forth in the respective SEQ ID NO.

In some embodiments, the variant TACI polypeptide comprises any of the mutations (amino acid substitutions) listed in Table 1. In some examples, the mutations (amino acid substitutions) are made in a reference TACI containing the sequence of amino acids set forth in SEQ ID NO: 122. In some examples, the mutations (amino acid substitutions) are made a reference TACI that contains the CRD1 and CRD2 domain of TACI, for example as set forth in SEQ ID NO: 1. In some examples, the mutations (amino acid substitutions) are made in a reference TACI that is further truncated by deletion of N-terminal and C-terminal amino acid residues to retain the CRD2, for example as set forth in SEQ ID NO: 13.

The use of the term “modification”, such as “substitution” or “mutation,” does not imply that the present embodiments are limited to a particular method of making the immunomodulatory proteins. A variant TACI polypeptide can be made, for example, by de novo peptide synthesis and thus does not necessarily require a modification, such as a “substitution” in the sense of altering a codon to encode for the modification, e.g. substitution. This principle also extends to the terms “addition” and “deletion” of an amino acid residue which likewise do not imply a particular method of making. The means by which the vTDs are designed or created is not limited to any particular method. In some embodiments, however, a wild-type or unmodified TD encoding nucleic acid is mutagenized from wild-type or unmodified TD genetic material and screened for desired specific binding activity, e.g. binding affinity, and/or alteration of NF-κB modulation or other functional activity. In some embodiments, a vTD is synthesized de novo utilizing protein or nucleic acid sequences available at any number of publicly available databases and then subsequently screened. The National Center for Biotechnology Information provides such information and its website is publicly accessible via the internet as is the UniProtKB database.

In some embodiments, the variant TACI polypeptide comprises an extracellular domain (ECD) sequence containing a CRD1 and CRD2, such as a variant TACI polypeptide set forth in any one of SEQ ID NOS: 2-12, 21, 22, 101-120. In some embodiments, the variant TACI polypeptide comprises a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 2-12, 21, 22, 101-120, and retains the amino acid modification(s), e.g. substitution(s) therein not present in the reference (e.g., unmodified or wild-type) TACI. In some embodiments, the variant TACI polypeptide comprises a specific binding fragment of any one of SEQ ID NOS: 2-12, 21, 22, 101-120, in which the specific binding fragment binds BAFF, APRIL or a BAFF/APRIL heterotrimer, and contains a contiguous sequence therein that contains the amino acid modification(s), e.g. substitution (s) therein not present in the reference (e.g., unmodified or wild-type) TACI.

In some embodiments, the variant TACI polypeptide consists or consists essentially of a variant TACI extracellular domain (ECD) sequence set forth in any one of SEQ ID NOS: 2-12, 21, 22, 101-120. In some embodiments, the variant TACI polypeptide consists or consists essentially of a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 2-12, 21, 22, 101-120, and retains the amino acid modification(s), e.g. substitution(s) therein not present in the reference (e.g., unmodified or wild-type) TACI. In some embodiments, the variant TACI polypeptide consists or consists essentially of a specific binding fragment of any one of SEQ ID NOS: 2-12, 21, 22, 101-120, in which the specific binding fragment binds BAFF, APRIL or an APRIL/BAFF heterotrimer and contains a contiguous sequence therein that contains the amino acid modification(s), e.g. substitution (s) therein not present in the reference (e.g., unmodified or wild-type) TACI.

In some embodiments, the variant TACI polypeptide comprises an extracellular domain (ECD) sequence containing a CRD2 but lacking the CRD1 of a reference TACI polypeptide, such as a variant TACI polypeptide set forth in any one of SEQ ID NOS: 14-20, 23-35, 92-100, 177-192. In some embodiments, the variant TACI polypeptide comprises a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 14-20, 23-35, 92-100, 177-192, and retains the amino acid modification(s), e.g. substitution(s) therein not present in the reference (e.g., unmodified or wild-type) TACI. In some embodiments, the variant TACI polypeptide comprises a specific binding fragment of any one of SEQ ID NOS: 14-20, 23-35, 92-100, 177-192 in which the specific binding fragment binds BAFF, APRIL or a BAFF/APRIL heterotrimer, and contains a contiguous sequence therein that contains the amino acid modification(s), e.g. substitution (s) therein not present in the reference (e.g., unmodified or wild-type) TACI.

In some embodiments, the variant TACI polypeptide consists or consists essentially of the sequence set forth in any one of SEQ ID NOS: 14-20, 23-35, 92-100, 177-192. In some embodiments, the variant TACI polypeptide consists or consists essentially of a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 14-20, 23-35, 92-100, 177-192, and retains the amino acid modification(s), e.g. substitution(s) therein not present in the reference (e.g., unmodified or wild-type) TACI. In some embodiments, the variant TACI polypeptide consists or consists essentially of a specific binding fragment of any one of SEQ ID NOS: 14-20, 23-35, 92-100, 177-192, in which the specific binding fragment binds BAFF, APRIL or a BAFF/APRIL heterotrimer, and contains a contiguous sequence therein that contains the amino acid modification(s), e.g. substitution (s) therein not present in the reference (e.g., unmodified or wild-type) TACI.

In some embodiments, the variant TACI polypeptide comprises the sequence set forth in SEQ ID NO:20. In some embodiments, the variant TACI polypeptide consists essentially of the sequence set forth in SEQ ID NO:20. In some embodiments, the variant TACI polypeptide consists of the sequence set forth in SEQ ID NO:20.

In some embodiments, the variant TACI polypeptide comprises the sequence set forth in SEQ ID NO:26. In some embodiments, the variant TACI polypeptide consists essentially of the sequence set forth in SEQ ID NO:26. In some embodiments, the variant TACI polypeptide consists of the sequence set forth in SEQ ID NO:26.

In some embodiments, the variant TACI polypeptide comprises the sequence set forth in SEQ ID NO:27. In some embodiments, the variant TACI polypeptide consists essentially of the sequence set forth in SEQ ID NO:27. In some embodiments, the variant TACI polypeptide consists of the sequence set forth in SEQ ID NO:27.

In some embodiments, the variant TACI polypeptide comprises the sequence set forth in SEQ ID NO:107. In some embodiments, the variant TACI polypeptide consists essentially of the sequence set forth in SEQ ID NO:107. In some embodiments, the variant TACI polypeptide consists of the sequence set forth in SEQ ID NO:107.

In some embodiments, the variant TACI polypeptide is encoded by a sequence of nucleotides set forth in any of SEQ ID NOS: 37-47, 56 or 57. In some embodiments, the variant TACI polypeptide is encoded by a sequence of nucleotides that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 37-47, 56 or 57, and retains the amino acid modification(s), e.g. substitution(s) therein not present in the reference (e.g., unmodified or wild-type) TACI. Also provided herein is a nucleic acid containing the sequence set forth in any of SEQ ID NOS: 37-47, 56 or 57 or a sequence that exhibits at least 90% identity, at least 91% identity, at least 92% identity, at least 93% identity, at least 94% identity, at least 95% identity, such as at least 96% identity, 97% identity, 98% identity, or 99% identity to any one of SEQ ID NOS: 37-47, 56 or 57.

In some embodiments, the variant TACI polypeptide is encoded by a sequence of nucleotides set forth in any of SEQ ID NOS: 49-55 or 58-70. In some embodiments, the variant TACI polypeptide is encoded by a sequence of nucleotides that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 49-55 or 58-70, and retains the amino acid modification(s), e.g. substitution(s) therein not present in the reference (e.g., unmodified or wild-type) TACI. Also provided herein is a nucleic acid containing the sequence set forth in any of SEQ ID NOS: 49-55 or 58-70 or a sequence that exhibits at least 90% identity, at least 91% identity, at least 92% identity, at least 93% identity, at least 94% identity, at least 95% identity, such as at least 96% identity, 97% identity, 98% identity, or 99% identity to any one of SEQ ID NOS: 49-55 or 58-70.

TABLE 1
Exemplary variant TACI

| | | ECD | | | |
|---|---|---|---|---|---|
| | | (CRD1/CRD2) | | ECD (CRD2) | |
| | | AA SEQ | NT SEQ | AA SEQ | NT SEQ |
| Name | Mutation(s) | ID NO | ID NO | ID NO | ID NO |
| 1 (WT) TACI CRD1/CRD2 | Wild-type | 1 | 36 | 13 | 48 |
| 13 (WT) TACI CRD2 | | | | | |
| 2 TACI CRD1/CRD2 | L82P | 2 | 37 | 92 | |
| 92 TACI CRD2 | | | | | |
| 3 TACI CRD1/CRD2 | D85E, K98T | 3 | 38 | 93 | |
| 93 TACI CRD2 | | | | | |
| 4 TACI CRD1/CRD2 | I87L, K98T | 4 | 39 | 94 | |
| 94 TACI CRD2 | | | | | |
| 5 TACI CRD1/CRD2 | R60G, Q75E, L82P | 5 | 40 | | |
| 6 TACI CRD1/CRD2 | R60G, C86Y | 6 | 41 | | |
| 7 TACI CRD1/CRD2 | A101D | 7 | 42 | 95 | |
| 95 TACI CRD2 | | | | | |
| 8 TACI CRD1/CRD2 | C86Y | 8 | 43 | 96 | |
| 96 TACI CRD2 | | | | | |
| 9 TACI CRD1/CRD2 | W40R, L82P, F103Y | 9 | 44 | | |
| 10 TACI CRD1/CRD2 | W40R, Q59R, T61P, K98T | 10 | 45 | | |
| 11 TACI CRD1/CRD2 | L82P, I87L | 11 | 46 | 97 | |
| 97 TACI CRD2 | | | | | |
| 12 TACI CRD1/CRD2 | G76S, P97S | 12 | 47 | 98 | |
| 98 TACI CRD2 | | | | | |
| 101 TACI CRD1/CRD2 | D85V | 101 | | 14 | 49 |
| 14 TACI CRD2 | | | | | |
| 102 TACI CRD1/CRD2 | E74V | 102 | | 15 | 50 |
| 15 TACI CRD2 | | | | | |
| 103 TACI CRD1/CRD2 | R84L | 103 | | 16 | 51 |
| 16 TACI CRD2 | | | | | |
| 104 TACI CRD1/CRD2 | K77E, R84L, F103Y | 104 | | 17 | 52 |
| 17 TACI CRD2 | | | | | |
| 105 TACI CRD1/CRD2 | Y79F, Q99E | 105 | | 18 | 53 |
| 18 TACI CRD2 | | | | | |
| 106 TACI CRD1/CRD2 | Y79F | 106 | | 19 | 54 |
| 19TACI CRD2 | | | | | |
| 107 TACI CRD1/CRD2 | R84G | 107 | | 20 | 55 |
| 20 TACI CRD2 | | | | | |
| 21 TACI CRD1/CRD2 | L83S, F103S | 21 | 56 | 99 | |
| 99 TACI CRD2 | | | | | |
| 22 TACI CRD1/CRD2 | L82H | 22 | 57 | 100 | |
| 100 TACI CRD2 | | | | | |
| 108 TACI CRD1/CRD2 | A101D | 108 | | 23 | 58 |
| 23 TACI CRD2 | | | | | |
| 109 TACI CRD1/CRD2 | K77E, R84Q | 109 | | 24 | 59 |
| 24 TACI CRD2 | | | | | |
| 110 TACI CRD1/CRD2 | K77E, A101D | 110 | | 25 | 60 |
| 25 TACI CRD2 | | | | | |
| 111 TACI CRD1/CRD2 | K77E, F78Y, Y102D | 111 | | 26 | 61 |
| 26 TACI CRD2 | | | | | |
| 112 TACI CRD1/CRD2 | Q75E, R84Q | 112 | | 27 | 62 |
| 27 TACI CRD2 | | | | | |
| 113 TACI CRD1/CRD2 | Q75R, R84G, I92V | 113 | | 28 | 63 |
| 28 TACI CRD2 | | | | | |
| 114 TACI CRD1/CRD2 | K77E, A101D, Y102D | 114 | | 29 | 64 |
| 29 TACI CRD2 | | | | | |
| 115 TACI CRD1/CRD2 | R84Q | 115 | | 30 | 65 |
| 30 TACI CRD2 | | | | | |
| 116 TACI CRD1/CRD2 | R84Q, S88N, A101D | 116 | | 31 | 66 |
| 31 TACI CRD2 | | | | | |
| 117 TACI CRD1/CRD2 | K77E | 117 | | 32 | 67 |
| 32 TACI CRD2 | | | | | |
| 118 TACI CRD1/CRD2 | R84Q, F103V | 118 | | 33 | 68 |
| 33 TACI CRD2 | | | | | |
| 119 TACI CRD1/CRD2 | K77E, Q95R, A101D | 119 | | 34 | 69 |
| 34 TACI CRD2 | | | | | |
| 120 TACI CRD1/CRD2 | I87M, A101D | 120 | | 35 | 70 |
| 35 TACI CRD2 | | | | | |
| 177 TACI CRD2 | Q75E | | | 177 | |
| 178 TACI CRD2 | Q75E, K77E | | | 178 | |
| 179 TACI CRD2 | Q75E, F78Y | | | 179 | |
| 180 TACI CRD2 | Q75E, A101D | | | 180 | |
| 181 TACI CRD2 | Q75E, Y102D | | | 181 | |
| 182 TACI CRD2 | K77E, F78Y, R84Q | | | 182 | |
| 183 TACI CRD2 | F78Y | | | 183 | |
| 184 TACI CRD2 | F78Y, R84Q | | | 184 | |
| 185 TACI CRD2 | F78Y, A101D | | | 185 | |
| 186 TACI CRD2 | F78Y, Y102D | | | 186 | |
| 187 TACI CRD2 | R84Q, A101D | | | 187 | |
| 188 TACI CRD2 | R84Q, Y102D | | | 188 | |
| 189 TACI CRD2 | A101D, Y102D | | | 189 | |
| 190 TACI CRD2 | Y102D | | | 190 | |
| 191 TACI CRD2 | K77E, F78Y | | | 191 | |
| 192 TACI CRD2 | K77E, Y102D | | | 192 | |

Among the provided formulation herein are those comprising a variant TACI-Fc polypeptide with improved binding affinity of the protein for BAFF and/or APRIL, such as any described herein and set forth in International Patent Publication Nos. WO2021226551 and WO 202223633. Such variant TACI polypeptides contain a variant TNFR domain (vTD, e.g., CRD2 domain) generated by directed evolution by affinity modification of the ectodomain of TACI to and have improved affinity for APRIL and/or BAFF. TACI-Fc fusion proteins of such variant TACI polypeptides linked with an immunoglobulin Fc provide for immunomodulatory proteins that suppress B cell activity and response. For instance, reformatted as a soluble Fc fusion protein, the affinity-matured TACI variant outputs exhibited inhibition of APRIL and BAFF, including with lower IC50 values than wild-type TACI-Fc and other therapeutic comparators (e.g., belimumab). Further, among such tested variant TACI-Fc fusion proteins are those that provide for rapid and significantly reduced key lymphocyte subsets including plasma cells, germinal center B cells, and follicular T helper cells. Further, tested variant molecules exhibited improved activities in mouse models, including significantly reduced autoantibodies and sialadenitis in the spontaneous SjS model, inhibited glomerular IgG deposition in the bm12-induced model of lupus, and potently suppressed anti-dsDNA autoantibodies, blood urea nitrogen levels, proteinuria, sialadenitis, kidney lesions and renal immune complex deposition in the NZB/W lupus model. Further, as compared to wild-type TACI-Fc, tested variant TACI-Fc fusions exhibited significantly and persistently decreased titers of serum IgM, IgG, and IgA antibodies in mice.

### 2. Fc Region

In some embodiments, a TACI polypeptide, such as any described above, is linked or fused to an immunoglobulin Fc region, such as any described herein. In some embodiments, the individual TACI-Fc fusion polypeptides of the TACI-Fc fusion protein form a multimer, e.g., a dimer. In some embodiments, the dimer is a homodimer in which the two polypeptides of the TACI-Fc fusion protein are the same. In some embodiments, the dimer is a heterodimer in which the two TACI-Fc fusion polypeptides of the TACI-Fc fusion protein are different. Exemplary Fc regions are described below.

The TACI polypeptide sequence (e.g. variant TACI polypeptide sequence) can be joined anywhere, but typically via its N- or C-terminus, to the N- or C-terminus of a Fc region to form a fusion polypeptide. The linkage can be direct or indirect via a linker. For example, when preparing a chimeric polypeptide containing an Fc region, nucleic acid encoding all or part of a TACI polypeptide sequence such as any described TACI ECD, including a variant TACI polypeptide sequence, can be operably linked to nucleic acid encoding the Fc region sequence, directly or indirectly or optionally via a linker domain. In some cases, the construct encodes a fusion polypeptide where the C-terminus of the TACI polypeptide sequence is joined to the N-terminus of the Fc region. In some instances, a construct can encode a fusion polypeptide where the N-terminus of the TACI polypeptide sequence is joined to the N- or C-terminus of the Fc region.

A polypeptide multimer contains two fusion polypeptides created by linking, directly or indirectly, two of the same or different TACI polypeptide sequences (e.g. two of the same or different variant TACI polypeptide sequences) directly or indirectly to an Fc region. In some examples, a gene fusion encoding the TACI polypeptide sequence (e.g. variant TACI polypeptide sequence) and Fc region is inserted into an appropriate expression vector. The resulting fusion protein can be expressed in host cells transformed with the recombinant expression vector, and allowed to assemble into multimers, where the Fc region domains interact to form multivalent polypeptides.

The resulting fusion proteins, such as dimers formed therefrom from individual polypeptide chains, can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation. For the generation of heterodimers, additional steps for purification can be necessary. For example, where two nucleic acids encoding different immunomodulatory proteins are transformed into cells, the formation of heterodimers must be biochemically achieved since immunomodulatory protein carrying the Fc-domain will be expressed as disulfide-linked homodimers as well. Thus, homodimers can be reduced under conditions that favor the disruption of interchain disulfides but do not affect intra-chain disulfides. In some cases, different TACI-Fc fusion protein monomers are mixed in equimolar amounts and oxidized to form a mixture of homo- and heterodimers. The components of this mixture are separated by chromatographic techniques. Alternatively, the formation of this type of heterodimer can be biased by genetically engineering and expressing immunomodulatory proteins containing Fc fusion molecules that contain one or more TACI variants using knob-into-hole methods as described.

In provided embodiments of a TACI-Fc, the immunoglobulin Fc region can be a wild-type Fc of an immunoglobulin, such as an IgG1 Fc. In some cases, the Fc region can be a variant Fc that lacks effector function (also called effectorless Fc). Exemplary Fc regions and variants thereof in provided TACI-Fc fusion proteins are described below.

In some embodiments, the Fc is murine or human Fc. In some embodiments, the Fc is a mammalian or human IgG1, IgG2, IgG3, or IgG4 Fc regions.

In some embodiments, the Fc region is or comprises the sequence set forth in any one of SEQ ID NOs: 71, 73, 75, 81, 82, 83, 134, 135, 136, 137, 138, 139, 140, 173, 174, 175, 176, 193, 218, 219, 220, or 221. In some embodiments, the Fc region is or is derived from an IgG1, such as set forth in any one of SEQ ID NOS: 71, 73, 75, 81, 82, 83, 134, 135, 136, 137, 139, 140, 173, 174, 175, 176, 193, 218, 220, or 221. In some embodiments, the Fc region is or is derived from an IgG2, such as any set forth in SEQ ID NO: 138 or 219. In some embodiments, the Fc region is or is derived from an IgG4, such as any set forth in SEQ ID NO: 139, 140 or 220. In some embodiments, an Fc region in Fc fusion proteins provided herein also can include an Fc region that exhibits at least about 85%, 86%, 87%, 88%, 89%, 90%, 91%, 92%, 93%, 94%, 95%, 96%, 97%, 98%, 99% to any of the above Fc regions.

In some embodiments, the Fc is derived from IgG1, such as human IgG1. In some embodiments, the Fc is an IgG1 Fc set forth in SEQ ID NO: 71 having an allotype containing residues Glu (E) and Met (M) at positions 356 and 358 by EU numbering. In some embodiments, the Fc comprises the amino acid sequence set forth in SEQ ID NO: 71 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 71. In other embodiments, the Fc is an IgG1 Fc that contains amino acids of the human G1 ml allotype, such as residues containing Asp (D) and Leu (L) at positions 356 and 358, e.g. as set forth in SEQ ID NO:81. Thus, in some cases, an Fc provided herein can contain amino acid substitutions E356D and M358L to reconstitute residues of allotype G1 ml. In some embodiments, the Fc comprises the amino acid sequence set forth in SEQ ID NO: 81 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 81.

In some embodiments, the Fc region has the amino acid sequence set forth in SEQ ID NO:81.

(SEQ ID NO: 81)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the Fc region comprises the amino acid sequence set forth in SEQ ID NO:81. In some embodiments, the Fc region consists of the amino acid sequence set forth in SEQ ID NO:81.

In some embodiments, the variant Fc comprises the sequence set forth in SEQ ID NO: 173. In some embodiments, the variant Fc comprises the sequence set forth in SEQ ID NO:174. In some embodiments, an Fc region used in a construct provided herein can further lack a C-terminal lysine residue.

In some embodiments, the Fe is derived from IgG2, such as human IgG2. In some embodiments, the Fc comprises the amino acid sequence set forth in SEQ ID NO: 138 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 138. In some embodiments, the Fc region is an IgG2 Fc region that has the sequence set forth in SEQ ID NO: 138. In some embodiments, the Fc region is an IgG2 Fc region that has the sequence set forth in SEQ ID NO: 219.

In some embodiments, the Fc is derived from IgG4, such as human IgG4. In some embodiments, the Fc comprises the amino acid sequence set forth in SEQ ID NO: 139 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 139. In some embodiments, the IgG4 Fc is a stabilized Fc in which the CH3 domain of human IgG4 is substituted with the CH3 domain of human IgG1 and which exhibits inhibited aggregate formation, an antibody in which the CH3 and CH2 domains of human IgG4 are substituted with the CH3 and CH2 domains of human IgG1, respectively, or an antibody in which arginine at position 409 indicated in the EU index proposed by Kabat et al. of human IgG4 is substituted with lysine and which exhibits inhibited aggregate formation (see e.g. U.S. Pat. No. 8,911,726. In some embodiments, the Fc is an IgG4 containing the S228P mutation, which has been shown to prevent recombination between a therapeutic antibody and an endogenous IgG4 by Fab-arm exchange (see e.g. Labrijin et al. (2009) Nat. Biotechnol., 27(8): 767-71.) In some embodiments, the Fc comprises the amino acid sequence set forth in SEQ ID NO: 140 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 140. In some embodiments, the Fc region is an IgG4 Fc region set forth in SEQ ID NO:140. In some embodiments, the Fc region is an IgG4 Fc region set forth in SEQ ID NO:220.

In some embodiments, the Fc region is a variant Fc region in which a wild-type Fc is modified by one or more amino acid substitutions to reduce effector activity or to render the Fc inert for Fc effector function. Exemplary effectorless or inert mutations include those described herein.

In some embodiments, the Fc region contains one more modifications that alter (e.g. reduce) one or more of its normal functions. In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be reduced or altered in an Fc for use with the provided Fc fusion proteins.

In some embodiments, one or more amino acid modifications may be introduced into the Fc region, thereby generating an Fc region variant. In some embodiments, the Fc region variant has decreased effector function. There are many examples of changes or mutations to Fc sequences that can alter effector function. For example, WO 00/42072, WO2006019447, WO2012125850, WO2015/107026, US2016/0017041 and Shields et al. J Biol. Chem. 9(2): 6591-6604 (2001) describe exemplary Fc variants with improved or diminished binding to FcRs. The contents of those publications are specifically incorporated herein by reference.

In some embodiments, the provided immunomodulatory proteins comprise an Fc region that exhibits reduced effector functions, which makes it a desirable candidate for applications in which the half-life of the immunomodulatory protein in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the immunomodulatory protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the immunomodulatory protein is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Immunomodulatory proteins with reduced effector function include those with substitution of one or more of Fc region residues 238, 265, 269, 270, 297, 327 and 329 by EU numbering (U.S. Pat. No. 6,737,056). Such Fc mutants include Fc mutants with substitutions at two or more of amino acid positions 265, 269, 270, 297 and 327 by EU numbering, including the so-called “DANA” Fc mutant with substitution of residues 265 and 297 to alanine (U.S. Pat. No. 7,332,581).

In some embodiments, the Fc region of immunomodulatory proteins has an Fc region in which any one or more of amino acids at positions 234, 235, 236, 237, 238, 239, 270, 297, 298, 325, and 329 (indicated by EU numbering) are substituted with different amino acids compared to the native Fc region. Such alterations of Fc region include, for example, alterations such as deglycosylated chains (N297A and N297Q), IgG1-N297G, IgG1-L234A/L235A, IgG1-L234A/L235E/G237A, IgG1-A325A/A330S/P331S, IgG1-C226S/C229S, IgG1-C226S/C229S/E233P/L234V/L235A, IgG1-E233P/L234V/L235A/G236del/S267K, IgG1-L234F/L235E/P331S, IgG1-S267E/L328F, IgG2-V234A/G237A, IgG2-H268Q/V309L/A330S/A331S, IgG4-L235A/G237A/E318A, and IgG4-L236E described in Current Opinion in Biotechnology (2009) 20 (6), 685-691; alterations such as G236R/L328R, L235G/G236R, N325A/L328R, and N325LL328R described in WO 2008/092117; amino acid insertions at positions 233, 234, 235, and 237 (indicated by EU numbering); and alterations at the sites described in WO 2000/042072.

Certain Fc variants with improved or diminished binding to FcRs are described. (See, e.g., U.S. Pat. No. 6,737,056; WO 2004/056312, WO2006019447 and Shields et al., J. Biol. Chem. 9(2): 6591-6604 (2001).)

In some embodiments, there is provided an immunomodulatory protein comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.) or WO2015107026. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434 by EU numbering, e.g., substitution of Fc region residue 434 (U.S. Pat. No. 7,371,826).

In some embodiments, the Fc region of the immunomodulatory protein comprises one or more amino acid substitutions C220S, C226S and/or C229S by EU numbering. In some embodiments, the Fc region of the immunomodulatory protein comprises one or more amino acid substitutions R292C and V302C. See also Duncan & Winter, Nature 322:738-40 (1988); U.S. Pat. Nos. 5,648,260; 5,624,821; and WO 94/29351 concerning other examples of Fc region variants.

In some embodiments, alterations are made in the Fc region that result in diminished C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, the variant Fc region comprising the one or more amino acid modifications (e.g. amino acid substitutions) is derived from a wild-type IgG1, such as a wild-type human IgG1. In some embodiments, the wild-type IgG1 Fc can be the Fc set forth in SEQ ID NO: 71 having an allotype containing residues Glu (E) and Met (M) at positions 356 and 358 by EU numbering. In some embodiments, the variant Fc region is derived from the amino acid sequence set forth in SEQ ID NO: 71. In other embodiments, the wild-type IgG1 Fc contains amino acids of the human G1 ml allotype, such as residues containing Asp (D) and Leu (L) at positions 356 and 358, e.g. as set forth in SEQ ID NO:81. Thus, in some cases, the variant Fc is derived from the amino acid sequence set forth in SEQ ID NO:81.

In some embodiments, the Fc region lacks the C-terminal lysine corresponding to position 232 of the wild-type or unmodified Fc set forth in SEQ ID NO: 71 or 81 (corresponding to K447del by EU numbering).

In some embodiments, the variant Fc region comprises a C5S amino acid modification of the wild-type or unmodified Fc region by numbering of SEQ ID NO: 71 (corresponding to C220S by EU numbering).

In some embodiments, the Fc region is a variant Fc that contains at least one amino acid substitution that is N82G by numbering of SEQ ID NO: 71 (corresponding to N297G by EU numbering). In some embodiments, the Fc further contains at least one amino acid substitution that is R77C or V87C by numbering of SEQ ID NO: 71 (corresponding to R292C or V302C by EU numbering). In some embodiments, the variant Fc region further comprises a C5S amino acid modification by numbering of SEQ ID NO: 71 (corresponding to C220S by EU numbering). For example, in some embodiments, the variant Fc region comprises the following amino acid modifications: N297G and one or more of the following amino acid modifications C220S, R292C or V302C by EU numbering (corresponding to N82G and one or more of the following amino acid modifications CSS, R77C or V87C with reference to SEQ ID NO:71), e.g., the Fc region comprises the sequence set forth in SEQ ID NO:82.

In some embodiments, the variant Fc contains the amino acid substitutions L234A/L235E/G237A, by EU numbering. In some embodiments, the variant Fc contains the amino acid substitutions A330S/P331S, by EU numbering. In some embodiments, the variant Fc contains the amino acid substitutions L234A/L235E/G237A/A330S/P331S (Gross et al. (2001) Immunity 15:289). In some embodiments, the variant Fc comprises the sequence set forth in SEQ ID NO: 175. In some embodiments, the variant Fc comprises the sequence set forth in SEQ ID NO:176. In some embodiments, an Fc region used in a construct provided herein can further lack a C-terminal lysine residue.

In some embodiments, the Fc region is a variant Fc that includes mutations L234A, L235E and G237A by EU numbering. In some embodiments, a wild-type Fc is further modified by the removal of one or more cysteine residue, such as by replacement of the cysteine residues to a serine residue at position 220 (C220S) by EU numbering. Exemplary inert Fc regions having reduced effector function are set forth in SEQ ID NO: 83 and SEQ ID NO:75, which are based on allotypes set forth in SEQ ID NO:71 or SEQ ID NO: 81, respectively. In some embodiments, an Fc region can further lack a C-terminal lysine residue. In some embodiments, the variant Fc region comprises one or more of the amino acid modifications C220S, L234A, L235E or G237A, e.g. the Fc region comprises the sequence set forth in SEQ ID NO:73, 75, 83 or 136. In some embodiments, the variant Fc comprises has the sequence set forth in SEQ ID NO: 73. In some embodiments, the variant Fc comprises has the sequence set forth in SEQ ID NO: 75. In some embodiments, the variant Fc comprises has the sequence set forth in SEQ ID NO: 83. In some embodiments, the variant Fc comprises has the sequence set forth in SEQ ID NO: 136.

In some embodiments, the Fc region is a variant Fc that has the sequence set forth in SEQ ID NO:73.

(SEQ ID NO: 73)
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the Fc region is an IgG1 Fc but does not contain a hinge sequence. In some embodiments, the IgG1 Fc region does not contain the hinge sequence EPKSC (SEQ ID NO:239). In some embodiments, the IgG1 Fc region does not contain a hinge sequence EPKSS (SEQ ID NO: 238).

In some embodiments, the Fc region is a variant Fc that has the sequence set forth in SEQ ID NO: 221.

(SEQ ID NO: 221)
DKTHTCPPCPAPEAEGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG

In some embodiments, the Fc region is a variant Fc region that comprises one or more of the amino acid modifications C220S, E233P, L234V, L235A, G236del or S267K, e.g. the Fc region comprises the sequence set forth in SEQ ID NO:134. In some embodiments, the Fc region lacks the C-terminal lysine corresponding to position 232 of the wild-type or unmodified Fc set forth in SEQ ID NO: 71 (corresponding to K447del by EU numbering). In some embodiments, the Fc region comprises the sequence set forth in SEQ ID NO:137.

In some embodiments, the Fc region is a variant Fc region that comprises one or more of the amino acid modifications C220S, R292C, N297G, V302C. In some embodiments, the Fc region lacks the C-terminal lysine corresponding to position 232 of the wild-type or unmodified Fc set forth in SEQ ID NO: 71 (corresponding to K447del by EU numbering). An exemplary variant Fc region is set forth in SEQ ID NO: 135.

In some embodiments, the variant Fc region comprises one or more of the amino acid modifications C220S/E233P/L234V/L235A/G236del/S267K. In some embodiments, the Fc region lacks the C-terminal lysine corresponding to position 232 of the wild-type or unmodified Fc set forth in SEQ ID NO: 71 (corresponding to K447del by EU numbering). An exemplary variant Fc region is set forth in SEQ ID NO: 137.

Examples of such Fc regions for inclusion in an immunomodulatory polypeptide are set forth in Table 2.

TABLE 2
Exemplary IgG1 Fc Regions, wild-type or variant (effectorless)

| | 356E/358M | 356D/358L |
|---|---|---|
| | allotype | allotype |
| Fc mutations (EU numbering) | SEQ ID NO | SEQ ID NO |
| (wild-type) | 71 | 81 (with |
| | | C220S, |
| | | K447del) |
| C220S, R292C, N297G, V302C | 82 | |
| C220S, R292C, N297G, V302C, K447del | 135 | |
| C220S, L234A, L235E, G237A | 83 | 75 |
| C220S, L234A, L235E, G237A, K447del | 136 | 73 |
| L234A, L235E, G237A, K447del, with deletion of | | 221 |
| hinge | | |
| C220S, E233P, L234V, L235A, G236del, S267K | 134 | |
| C220S/E233P/L234V/L235A/G236del/S267K/K447del | 137 | |
| L234A, L235E, G237A, A330S, P331S | | 176 |
| L234A, L235E, G237A, A330S, P331S, with deletion | | 175 |
| of hinge | | |

In some embodiments, the Fc region is a variant Fc region containing any combination of the Fc mutations in Table 2. In some embodiments, the Fc region is a variant Fc region having the sequence set forth in any one of the SEQ ID NOs in Table 2.

For example, a variant Fc region may be an effectorless Fc that exhibits reduced effector activity compared to a wild-type IgG1 set forth in SEQ ID NO:71 or SEQ ID NO:81. In some embodiments, the variant Fc comprises the sequence of amino acids set forth in any of SEQ ID NOS:75, 82, 83, 134, 73, 135, 136, or 137 or a sequence of amino acids that exhibits at least about 85%, 86%, 87%, 88%, 89%, 90%, 91%, 92%, 93%, 94%, 95%, 96%, 97%, 98%, 99% or more sequence identity to any of SEQ ID NOS: 75, 82, 83, 134, 73, 135, 136, or 137. In some embodiments, the variant Fc has the sequence set forth in SEQ ID NO: 73. In embodiments, when produced and expressed from cells, the provided immunomodulatory protein (e.g. TACI-Fc fusion) is a homodimer containing two identical polypeptide chains.

In some embodiments, the immunomodulatory protein contains a first immunomodulatory Fc fusion polypeptide and a second immunomodulatory Fc fusion polypeptide in which the first and second polypeptide are different. In some embodiments, a first Fc polypeptide fusion contains an Fc region and one or more variant TACI polypeptide sequence and a second polypeptide fusion contains an Fc region and one or more TACI polypeptide sequence. In such embodiments, the Fc region can be a region that promotes or facilitates formation of heterodimers.

In some embodiments, the Fc domain of one or both of the first and second immunomodulatory Fc fusion polypeptides comprise a modification (e.g. substitution) such that the interface of the Fc molecule is modified to facilitate and/or promote heterodimerization. Methods to promote heterodimerization of Fc chains include mutagenesis of the Fc region, such as by including a set of knob-into-hole mutations or including mutations to effect electrostatic steering of the Fc to favor attractive interactions among different polypeptide chains. In some embodiments, the Fc region of the heterodimeric molecule additionally can contain one or more other Fc mutation, such as any described above. In some embodiments, the heterodimer molecule contains an Fc region with a mutation that reduces effector function. In some embodiments, such Fc regions contain mutations C220S, L234A, L235E and/or G237A by EU numbering. In some embodiments, any of the above mutations in an Fc backbone can be made in an allotype containing residues Glu (E) and Met (M) at positions 356 and 358 by EU numbering. In other embodiments, any of the above mutations in an Fc backbone can be made in an allotype containing residue Asp (D) and Leu (L) at positions 356 and 358 by EU numbering.

In addition, an Fc region can include an amino acid sequence comprising a protuberance complementary to an amino acid sequence comprising a hole, such as is described, for example, in U.S. Pat. No. 5,731,168; International Patent Pub. Nos. WO 98/50431 and WO 2005/063816; Ridgway et al. (1996) Protein Engineering, 9:617-621. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or tryptophan). Compensatory cavities of identical or similar size to the protuberances are optionally created on the interface of the second polypeptide by replacing large amino acid side chains with smaller ones (e.g., alanine or threonine).

In some embodiments, modifications include introduction of a protuberance (knob) into a first Fc polypeptide and a cavity (hole) into a second Fc polypeptide such that the protuberance is positionable in the cavity to promote complexing of the first and second Fc-containing polypeptides. Amino acids targeted for replacement and/or modification to create protuberances or cavities in a polypeptide are typically interface amino acids that interact or contact with one or more amino acids in the interface of a second polypeptide.

In some embodiments, a first polypeptide that is modified to contain protuberance (knob) amino acids include replacement of a native or original amino acid with an amino acid that has at least one side chain which projects from the interface of the first polypeptide and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, a second polypeptide that is modified to contain a cavity (hole) is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 A2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

In some embodiments, the heterodimeric molecule contains a T366W mutation in the CH3 domain of the “knobs chain” and T366S, L368A, Y407V mutations in the CH3 domain of the “hole chain.” In some cases, an additional interchain disulfide bridge between the CH3 domains can also be used (Merchant, A. M., et al., Nature Biotech. 16 (1998) 677-681) e.g. by introducing a Y349C mutation into the CH3 domain of the “knobs” or “hole” chain and a E356C mutation or a S354C mutation into the CH3 domain of the other chain. In some embodiments, the heterodimeric molecule contains S354C, T366W mutations in one of the two CH3 domains and Y349C, T366S, L368A, Y407V mutations in the other of the two CH3 domains. For example, the knob Fc may contain the sequence set forth in SEQ ID NO: 89, containing S354C and T366W, and a hole Fc set forth in SEQ ID NO: 90, containing mutations Y349C, T366S, L368A and Y407V). In some embodiments, the heterodimeric molecule comprises E356C, T366W mutations in one of the two CH3 domains and Y349C, T366S, L368A, Y407V mutations in the other of the two CH3 domains. In some embodiments, the heterodimeric molecule comprises Y349C, T366W mutations in one of the two CH3 domains and E356C, T366S, L368A, Y407V mutations in the other of the two CH3 domains. In some embodiments, the heterodimeric molecule comprises Y349C, T366W mutations in one of the two CH3 domains and S354C, T366S, L368A, Y407V mutations in the other of the two CH3 domains. Examples of other knobs-in-holes technologies are known in the art, e.g. as described by EP 1 870 459 A1.

In some embodiments, an Fc variant containing CH3 protuberance (knob) or cavity (hole) modifications can be joined to a multi-domain immunomodulatory polypeptide anywhere, but typically via its N- or C-terminus, to the N- or C-terminus of the one or more TACI polypeptide sequence (e.g. variant TACI polypeptide sequence), such as to form a fusion polypeptide. The linkage can be direct or indirect via a linker. Typically, a knob and hole molecule is generated by co-expression of a first immunomodulatory polypeptide linked to an Fc variant containing CH3 protuberance modification(s) with a second immunomodulatory polypeptide linked to an Fc variant containing CH3 cavity modification(s).

Exemplary sequences for knob and hole Fc polypeptides are set forth in SEQ ID NOs: 128, and 129, respectively. In some embodiments, the knob or hold Fc region lacks the C-terminal lysine corresponding to position 232 of the wild-type or unmodified Fc set forth in SEQ ID NO: 71 (corresponding to K447del by EU numbering). Exemplary sequences for knob and hole Fc polypeptides are set forth in SEQ ID NOs: 89 and 90, respectively.

In some embodiment, individual polypeptide of a multi-domain polypeptide or individual polypeptides of a single-domain polypeptide are linked to a multimerization domain that forms an immunomodulatory protein is a trimer, tetramer or pentamer. In some embodiments, the individual polypeptides of such a molecule are the same. In some embodiments, such a multimerization domain is a cartilage oligomeric matrix protein (COMP) assembly domain, a vasodilator-stimulated phosphoprotein (VASP) tetramerization domain or a ZymoZipper (ZZ) 12.6 domain.

In some embodiments, the multimerization domain is a portion of the cartilage oligomeric matrix protein (COMP) assembly domain (Voulgaraki et al., Immunology (2005) 115(3):337-346. In some examples, the COMP is or contains an amino acid sequence as set forth in SEQ ID NO: 146 (e.g. amino acids 29-72 of the full length COMP, Uniprot accession number P49747) or a sequence that has about 85%, 85%, 87%, 88%, 89%, 90%, 91%, 92%, 93%, 94%, 95%, 96%, 97%, 98%, 99% or more sequence identity to SEQ ID NO: 146.

In some embodiments, the multimerization domain is a vasodilator-stimulated phosphoprotein (VASP) tetramerization domain (Bachmann et al., J Biol Chem (1999) 274(33):23549-23557). In some embodiments, the VASP is or contains an amino acid sequence as set forth in SEQ ID NO: 147 (e.g. amino acids 343-375 of the full length VASP; Uniprot accession number P50552) or a sequence that has about 85%, 85%, 87%, 88%, 89%, 90%, 91%, 92%, 93%, 94%, 95%, 96%, 97%, 98%, 99% or more sequence identity to SEQ ID NO: 147.

In some embodiments, a TACI polypeptide sequence (e.g. variant TACI polypeptide sequence) is joined to the multimerization domain (e.g. Fc region) via a linker, such as a peptide linker. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length.

In some embodiments, the linker is (in one-letter amino acid code): GGGGS (“4GS”; SEQ ID NO: 77) or multimers of the 4GS linker, such as repeats of 2, 3, 4, or 5 4GS linkers. In some embodiments, the peptide linker is the peptide linker is (GGGGS)₂ (SEQ ID NO: 78), (GGGGS)₃ (SEQ ID NO: 79), (GGGGS)₄ (SEQ ID NO: 84) or (GGGGS)₅ (SEQ ID NO: 91). In some embodiments, the linker also can include a series of alanine residues alone or in addition to another peptide linker (such as a 4GS linker or multimer thereof). In some embodiments, the linker (in one-letter amino acid code) is GSGGGGS (SEQ ID NO: 74) or GGGGSSA (SEQ ID NO: 80). In some examples, the linker is a 2×GGGGS followed by three alanines (GGGGSGGGGSAAA; SEQ ID NO:133). In some examples, the linker is set forth in SEQ ID NO: 194 or 195.

In some embodiments, the TACI polypeptide, such as the variant TACI polypeptide, is directly linked to the Fc sequence. In some embodiments, the TACI polypeptide, such as the variant TACI polypeptide, is indirectly linked to the Fc sequence, such as via a linker. In some embodiments, one or more “peptide linkers” link the TACI polypeptide (e.g. variant TACI polypeptide) and the Fc region. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. Exemplary linkers include any linker as described herein.

### 3. Exemplary TACI-Fc Fusion Proteins

In some embodiments, the TACI-Fc fusion protein has the structure TACI polypeptide (TACI)-Linker-Fc region. In some embodiments, the immunomodulatory protein is a homodimer of two identical copies of the TACI-Fc fusion protein. For instance, interactions between Fc regions of the two identical polypeptide fusions form covalent disulfide bonds to result in a dimeric molecule containing two TACI polypeptides (e.g. two variant TACI polypeptides).

In some embodiments, there is provided a TACI-Fc fusion protein containing in order a TACI polypeptide, e.g. any as described above, a linker and an Fc region. In some embodiments, each TACI polypeptide of the TACI Fc fusion is a truncated wild-type TACI polypeptide, such as any as described. In some embodiments, the TACI polypeptide of the TACI Fc fusion is set forth in SEQ ID NO: 13. In some embodiments, provided herein is a TACI-Fc fusion sequence that contains (1) a TACI ECD sequence that comprises the sequence set forth in SEQ ID NO:13, and (2) an immunoglobulin Fc region. In some embodiments, provided herein is a TACI-Fc fusion sequence that contains (1) a TACI ECD sequence that consists or consists essentially of the sequence set forth in SEQ ID NO:13, and (2) an immunoglobulin Fc region. The linker may be any as described. In some embodiments, the linker is GSGGGGS (SEQ ID NO: 74). In some embodiments, the linker is GS(G4S)₂ (SEQ ID NO: 194). The Fc region may be any Fc region as described. In some embodiments, the Fc region is a wild-type IgG1 Fc set forth in SEQ ID NO:81. In some embodiments, the Fc region is a variant Fc set forth in SEQ ID NO: 73.

In some embodiments, the TACI-Fc fusion protein has the sequence set forth in SEQ ID NO:171. In some embodiments, the TACI-Fc fusion protein has the sequence set forth in SEQ ID NO:197. In some embodiments, the TACI-Fc fusion is encoded by the sequence set forth in SEQ ID NO:208.

(SEQ ID NO: 171)
SLSCRKEQGKFYDHLLRDCISCASICGQHPKQCAYFCENKLRSGSGGGGS
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO:172.

(SEQ ID NO: 172)
SLSCRKEQGKFYDHLLRDCISCASICGQHPKQCAYFCENKLRSGSGGGGS
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO: 196, and encoded the sequence set forth in SEQ ID NO:207.

In other embodiments, the formulations provided herein can contain TACI-Fc immunomodulators that contain a wild-type TACI polypeptide portion of the ECD such as, for example, atacicept or telitacicept. In some embodiments, the formulations provided herein can contain a TACI-Fc set forth in SEQ ID NO:131; corresponding to the TACI ECD portion in telitacicept, or a TACI-Fc set forth in SEQ ID NO:130; corresponding to the TACI ECD portion in atacicept, SEQ ID NO:132.

In some embodiments, the TACI-Fc fusion is a variant TACI-Fc fusion containing or consisting of any of the above-described variant TACI polypeptides and an immunoglobulin Fc region.

In some embodiments, provided herein is a variant TACI-Fc fusion sequence that contains (1) a TACI ECD sequence containing a CRD1 and a CRD2, for example a TACI sequence that contains the sequence set forth in any one of SEQ ID NOS: 2-12, 21, 22, 101-120, and (2) an immunoglobulin Fc region. In some embodiments, provided herein is a variant TACI-Fc fusion sequence that contains (1) a TACI ECD sequence containing a CRD1 and a CRD2, for example a TACI sequence that consist or consists essentially of the sequence set forth in any one of SEQ ID NOS: 2-12, 21, 22, 101-120, and (2) an immunoglobulin Fc region.

In some embodiments, provided herein is a variant TACI-Fc fusion sequence that contains (1) a TACI ECD sequence containing the CRD2 but lacking the CRD1 domain, for example a TACI sequence that contains the sequence set forth in any one of SEQ ID NOS: 14-20, 23-35, 92-100, 177-192 and (2) an immunoglobulin Fc region. In some embodiments, provided herein is a variant TACI-Fc fusion sequence that contains (1) a TACI ECD sequence containing the CRD2 domain but lacking the CRD1 domain, for example a TACI sequence that consists or consists essentially of the sequence set forth in any one of SEQ ID NOS: 14-20, 23-35, 92-100, 177-192 and (2) an immunoglobulin Fc region.

In some embodiments, the TACI polypeptide is a variant TACI polypeptide. In some embodiments, there is provided a variant TACI-Fc fusion protein containing in order a variant TACI polypeptide, e.g. any as described above, a linker and an Fc region. In some embodiments, the TACI polypeptide of the TACI Fc fusion is a variant TACI polypeptide, such as any as described. In some embodiments, the variant TACI of the variant TACI Fc fusion is set forth in any one of SEQ ID NOS: 2-12, 21, 22, or 101-120. In some embodiments, the variant TACI of the variant TACI Fc fusion is set forth in any one of SEQ ID NOS: 14-20, 23-35, 92-100 or 177-192. In some embodiments, the linker is GSGGGGS (SEQ ID NO: 74). In some embodiments, the linker is GS(G4S)₂ (SEQ ID NO: 194). In some embodiments, the Fc region is a wild-type IgG1 Fe set forth in SEQ ID NO:81. In some embodiments, the Fc region is a variant Fe set forth in SEQ ID NO: 73.

In some embodiments, the TACI-Fc fusion protein has the sequence of amino acids set forth in any one of SEQ ID NOS: 167-170, 200, or 222-237.

In some embodiments, the TACI-Fc fusion protein has the sequence set forth in SEQ ID NO:167.

(SEQ ID NO: 167)
SLSCRKEQGEYYDHLLRDCISCASICGQHPKQCADFCENKLRSGSGGGGS
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments of the provided formulations, the TACI-Fc fusion protein is a homodimer of two polypeptide of the formula TACI-linker-Fc in which the TACI is a variant TACI that is a portion of the extracellular domain composed of the CRD2 TNF receptor domain set forth in SEQ ID NO:13 in which is present amino acid substitutions K77E, F78Y and Y102D. In some embodiments, the variant TACI is set forth in SEQ ID NO:26. In embodiments of any of the described TACI-Fc fusion proteins, the variant TACI is linked to the Fc domain via a linker. In some of any of the provided methods or uses, the TACI-Fc fusion protein has the sequence set forth in SEQ ID NO: 167. In some of any embodiments, the TACI-Fc fusion protein has the sequence set forth in SEQ ID NO: 168.

In some embodiments, the TACI-Fc fusion is encoded by the sequence set forth in SEQ ID NO:211.

In some embodiments, the TACI-Fc fusion protein has the sequence set forth in SEQ ID NO:168.

(SEQ ID NO: 168)
SLSCRKEQGEYYDHLLRDCISCASICGQHPKQCADFCENKLRSGSGGGGS
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO: 169.

(SEQ ID NO: 169)
SLSCRKEEGKFYDHLLQDCISCASICGQHPKQCAYFCENKLRSGSGGGGS
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO:170

(SEQ ID NO: 170)
SLSCRKEEGKFYDHLLQDCISCASICGQHPKQCAYFCENKLRSGSGGGGS
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

Provided herein is a TACI (26)-Fc_73 homodimer of two identical variant TACI-Fc fusion proteins containing a variant of the TACI Cysteine Rich Domain 2 (CRD2) set forth in SEQ ID NO:26 designed to neutralize the B-cell stimulatory activity of APRIL and BAFF. The TACI (26)-Fc_73 homodimer is a dimer consisting of 2 identical receptor Fc-fusion protein chains, each with a variant TACI CRD2 domain human Fc-fusion set forth in SEQ ID NO:167, linked by covalent disulfide bonds.

Provided herein is a TACI (26)-Fc_81 homodimer of two identical variant TACI-Fc fusion proteins containing a variant of the TACI Cysteine Rich Domain 2 (CRD2) set forth in SEQ ID NO:26 designed to neutralize the B-cell stimulatory activity of APRIL and BAFF. The TACI (26)-Fc_81 homodimer is a dimer consisting of 2 identical receptor Fc-fusion protein chains, each with a variant TACI CRD2 domain human Fc-fusion set forth in SEQ ID NO:168, linked by covalent disulfide bonds.

Provided herein is a TACI (27)-Fc_73 homodimer of two identical variant TACI-Fc fusion proteins containing a variant of the TACI Cysteine Rich Domain 2 (CRD2) set forth in SEQ ID NO:27 designed to neutralize the B-cell stimulatory activity of APRIL and BAFF. The TACI (27)-Fc_73 homodimer is a dimer consisting of 2 identical receptor Fc-fusion protein chains, each with a variant TACI CRD2 domain human Fc-fusion set forth in SEQ ID NO:169, linked by covalent disulfide bonds.

Provided herein is a TACI (27)-Fc_81 homodimer of two identical variant TACI-Fc fusion proteins containing a variant of the TACI Cysteine Rich Domain 2 (CRD2) set forth in SEQ ID NO:27 designed to neutralize the B-cell stimulatory activity of APRIL and BAFF. The TACI (27)-Fc_81 homodimer is a dimer consisting of 2 identical receptor Fc-fusion protein chains, each with a variant TACI CRD2 domain human Fc-fusion set forth in SEQ ID NO:170, linked by covalent disulfide bonds.

In some embodiments, the TACI-Fc fusion protein contains multiple copies of a TACI polypeptide sequence (e.g. variant TACI-polypeptide sequence), such as 2, 3 or 4 TACI polypeptide sequences. In some embodiments, the TACI-Fc fusion proteins contain two TACI polypeptide sequences (e.g. two variant TACI polypeptide sequences). In some cases, the TACI polypeptide sequences may be linked directly or may be linked indirectly via a linker, such as a peptide linker including any as described. In such an example, one of the TACI polypeptide sequence is joined or linked to the Fc region, such as either to the N- or C-terminus of the Fc region. In other cases, the TACI polypeptide sequences may be separated from each other by the Fc region and each joined individually to the N- or C-terminus of the Fc region. The linkage to the Fc region may be direct or may be indirect via a linker, such as a peptide linker including any as described.

In some embodiments, the TACI polypeptide sequences (e.g. variant TACI polypeptide sequences) may be arranged in order in the fusion protein in tandem (hereinafter called a “tandem” Fc fusion construct). In some embodiments, the TACI-Fc fusion protein has the structure: (TACI)-Linker-(TACI)-Linker-Fc region. In some embodiments, the immunomodulatory protein is a tetravalent molecule that is a homodimer of two identical copies of the TACI-Fc fusion protein. For instance, interactions between Fc regions of the two identical polypeptide fusions form covalent disulfide bonds to result in a dimeric molecule containing four TACI polypeptides (e.g. four variant TACI polypeptides).

In some embodiments, there is provided a TACI-Fc fusion protein containing in order a TACI polypeptide, e.g. any as described above; a linker; another TACI polypeptide, e.g. any as described; and an Fc region. In some embodiments, each TACI polypeptide of the TACI Fc fusion is a truncated wild-type TACI polypeptide, such as any as described. In some embodiments, each TACI polypeptide of the TACI Fc fusion is set forth in SEQ ID NO: 13. In some embodiments, each TACI polypeptide of the TACI Fc fusion is a variant TACI polypeptide, such as any as described. In some embodiments, each TACI polypeptide of the TACI Fc fusion is a variant TACI set forth in any one of SEQ ID NOS: 2-12, 21, 22, or 101-120. In some embodiments, each TACI polypeptide of the TACI Fc fusion is a variant TACI set forth in any one of SEQ ID NOS: 14-20, 23-35, 92-100 or 177-192. The linkers may be any as described. In some embodiments, the linker is GSGGGGS (SEQ ID NO: 74). The Fc region may be any Fc region as described. In some embodiments, the Fc region is a wild-type IgG1 Fc set forth in SEQ ID NO:81. In some embodiments, the Fc region is a variant Fc set forth in SEQ ID NO: 73. In some embodiments, the TACI-Fc fusion protein has the sequence set forth in SEQ ID NO:198, and encoded by a sequence set forth in SEQ ID NO:209.

In some embodiments, the TACI polypeptide sequences (e.g. variant TACI polypeptide sequences) may be separated in the fusion protein by the Fc region in which the Fc region is positioned between the two TACI polypeptide sequences (hereinafter called a “barbell” Fc fusion construct). In some embodiments, the TACI-Fc fusion protein has the structure: (TACI)-Linker-Fc region-Linker-(TACI). In some embodiments, the linkers may be the same or different. In some embodiments, the immunomodulatory protein is a tetravalent molecule that is a homodimer of two identical copies of the TACI-Fc fusion protein. For instance, interactions between Fc regions of the two identical polypeptide fusions form covalent disulfide bonds to result in a dimeric molecule containing four TACI polypeptides (e.g. four variant TACI polypeptides).

In some embodiments, there is provided a TACI-Fc fusion protein containing in order a TACI polypeptide, e.g. any as described above; a linker; an Fc region; a linker; and another TACI polypeptide, e.g. any as described. In some embodiments, each TACI polypeptide of the TACI Fc fusion is a truncated wild-type TACI polypeptide, such as any as described. In some embodiments, each TACI polypeptide of the TACI Fc fusion is set forth in SEQ ID NO: 13. In some embodiments, each TACI polypeptide of the TACI Fc fusion is a variant TACI polypeptide, such as any as described. In some embodiments, each TACI polypeptide of the TACI Fc fusion is a variant TACI set forth in any one of SEQ ID NOS: 2-12, 21, 22, or 101-120. In some embodiments, each TACI polypeptide of the TACI Fc fusion is a variant TACI set forth in any one of SEQ ID NOS: 14-20, 23-35, 92-100 or 177-192. The linkers may be any as described, and may be the same or different. In some embodiments, the first linker is GSGGGGS (SEQ ID NO: 74) and the second linker is (GGGGS)₄ (SEQ ID NO: 84). The Fc region may be any Fc region as described. In some embodiments, the Fc region is a wild-type IgG1 Fc set forth in SEQ ID NO:81. In some embodiments, the Fc region is a variant Fc set forth in SEQ ID NO: 73. In some embodiments, the TACI-Fc fusion protein has the sequence set forth in SEQ ID NO:201, and encoded by a sequence set forth in SEQ ID NO:212. In some embodiments, the TACI-Fc fusion protein has the sequence set forth in SEQ ID NO:202, and encoded by a sequence set forth in SEQ ID NO:213.

In some embodiments, there is a provided a TACI-Fc fusion protein that is a dimer formed by two identical TACI polypeptides (e.g. variant TACI polypeptide) as described linked to an Fc domain. In some embodiments, identical species (also referred to as copies) of any of the provided TACI-Fc fusion polypeptides, e.g. variant TACI-Fc fusion, will be dimerized to create a homodimer. In some embodiments, the dimer is a homodimer in which the two TACI-Fc polypeptides, e.g. variant TACI-Fc polypeptides, are the same. For generating a homodimeric Fc molecule, the Fc region is one that is capable of forming a homodimer with a matched Fc region by co-expression of the individual Fc regions in a cell. In some embodiments, dimerization is mediated by covalent disulfide bond(s) formed between the Fc regions of the polypeptide fusions.

### 4. Concentration

The formulation herein can be prepared at a high concentration of a TACI-Fc fusion protein. In the present formulation, the viscosity of the formulation is low, such that it is sufficient for subcutaneous administration, despite the high concentration of the TACI-Fc fusion protein in the formulation. In some embodiments, the concentration of the TACI-Fc protein in the formulation is at or about 100 mg/mL and up to at or about 230 mg/mL. In some embodiments, the concentration of the TACI-Fc protein in the formulation is at or about 140 mg/mL and up to at or about 200 mg/mL. In embodiments herein, the formulation contains a high concentration of the TACI-Fc fusion protein and the majority, such as at least 90% or at least 95% or at least 97% or at least 98% or at least 99%, of the TACI-Fc fusion protein in the formulation is the main, homodimer form.

In some embodiments, the concentration of the TACI-Fc fusion protein in the formulation is at least about 100 mg/mL. In some embodiments, the concentration of the TACI-Fc fusion protein in the formulation is at least about 140 mg/mL. In some embodiments, the concentration of the TACI-Fc fusion protein in the formulation is at least about 160 mg/mL. In some embodiments, the concentration of the TACI-Fc protein in the formulation is between at or about 100 mg/mL and 200 mg/mL, between at or about 100 mg/mL and 175 mg/mL, between at or about 100 mg/mL and 150 mg/mL, between at or about 120 mg/mL and 200 mg/mL, between at or about 120 mg/mL and 175 mg/mL, between at or about 120 mg/mL and 150 mg/mL, between at or about 140 mg/mL and 200 mg/mL, between at or about 160 mg/mL and 200 mg/mL, between at or about 140 mg/mL and 230 mg/mL, between at or about 140 mg/mL and 230 mg/mL, between at or about 140 mg/mL and 175 mg/mL, between at or about 140 mg/mL and 150 mg/mL, between at or about 150 mg/mL and 200 mg/mL, between at or about 150 mg/mL and 175 mg/mL, between at or about 150 mg/mL and 160 mg/mL, between at or about 160 mg/mL and 200 mg/mL, or between at or about 160 mg/mL and 175 mg/mL. In some embodiments, the concentration of the TACI-Fc fusion protein in the formulation is at or about 120 mg/mL, at or about 140 mg/mL, at or about 160 mg/mL, at or about 165 mg/mL, at or about 170 mg/mL, at or about 175 mg/mL, at or about 180 mg/mL, or at or about 190 mg/mL, or at or about 200 mg/mL, or at or about 210 mg/mL, or at or about 220 mg/mL, at or about 230 mg/mL, or any value between any of the foregoing. In some embodiments, the concentration of the TACI-Fc fusion protein in the formulation is at or about 160 mg/mL. In some embodiments, the concentration of the TACI-Fc fusion protein in the formulation is at or about 175 mg/mL.

Exemplified herein are TACI-Fc formulations where the TACI-Fc protein is formulated at high protein concentrations. In some embodiments, the pharmaceutical formulation is formulated to contain an amount of a TACI-Fc fusion of from at or about 100 mg to at or about 250 mg, such as from at or about 100 mg to at or about 175 mg, from at or about 100 mg to at or about 150 mg, from at or about 100 mg to at or about 250 mg, from at or about 125 mg to at or about 150 mg, from at or about 125 mg to at or about 175 mg, from at or about 125 mg to at or about 200 mg, from at or about 125 mg to at or about 250 mg, from at or about 150 mg to at or about 175 mg, from at or about 140 mg to at or about 200 mg, from at or about 150 mg to at or about 200 mg, from at or about 150 mg to at or about 230 mg, from at or about 150 mg to at or about 250 mg, from at or about 155 mg and at or about 195 mg, from at or about 155 mg and at or about 170 mg, from at or about 155 mg and at or about 175 mg, from at or about 155 mg and at or about 180 mg, from at or about 155 mg and at or about 185 mg, from at or about 155 mg and at or about 190 mg, from at or about 158 mg and at or about 195 mg, from at or about from at or about 160 mg to at or about 175 mg, from at or about 160 mg to at or about 200 mg, from at or about 160 mg to at or about 230 mg, or from at or about 160 mg to at or about 250 mg. In some embodiments, the pharmaceutical formulation is formulated to contain an amount of a TACI-Fc fusion protein that is at or about 100 mg, at or about 120 mg, at or about 125 mg, at or about 130 mg, at or about 140 mg, at or about 150 mg, at or about 160 mg, at or about 170 mg, at or about 175 mg, at or about 180 mg, or at or about 200 mg, or at or about 210 mg, or at or about 220 mg, or at or about 230 mg, or at or about 240 mg, or at or about 250 mg or any value between any of the foregoing. In some embodiments, the pharmaceutical formulation is formulated to contain an amount of a TACI-Fc fusion protein that is at or about 160 mg. In some embodiments, the pharmaceutical formulation is formulated to contain an amount of a TACI-Fc fusion protein that is at or about 175 mg. In some embodiments, the pharmaceutical formulation is formulated to contain an amount of a TACI-Fc fusion protein that is at or about 200 mg. In some embodiments, the pharmaceutical formulation is formulated in a volume that is from at or about 0.5 mL to at or about 10 mL, such as from at or about 0.5 mL to at or about 5 mL, from at or about 0.5 mL to at or about 2 mL, from at or about 0.5 mL to at or about 1 mL, from at or about 1 mL to at or about 10 mL, from at or about 1 mL to at or about 5 mL or from at or about 5 mL to at or about 10 mL. In some embodiments, the pharmaceutical formulation is formulated in a volume that is at or about 0.5 mL, at or about 1 mL, at or about 2 mL, at or about 2.5 mL, at or about 3 mL, at or about 4 mL, at or about 5 mL, at or about 6 mL, at or about 7 mL, at or about 8 mL, at or about 9 mL or at or about 10 mL. In some embodiments, the formulation is formulated in a volume that is at or about 0.5 mL, 0.6 mL, 0.7 mL, 0.8 mL, 0.9 mL, 1.0 mL, 1.2 mL, 1.4. mL, 1.6 mL, 1.8 mL or 2.0 mL, or any value between any of the foregoing. In some embodiments, the pharmaceutical formulation is formulated in a volume that is at or about 1 mL. In some embodiments, the pharmaceutical formulation is formulated in a volume that is at or about 1.5 mL. In some embodiments, the pharmaceutical formulation is formulated in a volume that is at or about 1.75 mL. In some embodiments, the pharmaceutical formulation is formulated in a volume that is at or about 2 mL.

In some of any embodiments, when administering a formulation containing a TACI-Fc fusion protein the formulation may be diluted in a corresponding formulation buffer to achieve a lower concentration prior to administration. In these examples, a corresponding formulation buffer contains the same components as the TACI-Fc formulation buffer but does not contain the TACI-Fc fusion protein. In some of any embodiments, when administering a formulation containing a TACI-Fc fusion protein at a concentration greater than 200 mg/mL it may be diluted in a corresponding formulation buffer to achieve a lower concentration prior to administration. In some of any embodiments the formulation buffer is a formulation buffer described herein.

### B. Buffer and Formulation pH

In some embodiments, the pharmaceutical formulation is a liquid, for example immunomodulatory proteins (e.g. TACI-Fc fusion protein) dissolved in an aqueous solution (such as water). In some embodiments, the liquid formulation is not lyophilized prior to administration and has not previously been lyophilized and reconstituted as a liquid. In embodiments herein, the formulation is prepared in the presence of a buffering agent and at a pH wherein the TACI-Fc in the formulation is stable. In some embodiments, the formulation provided herein is a solid. In some embodiments, the formulation provided herein is a frozen liquid that is solid (i.e., a lyophilized formulation).

In some cases, it may be advantageous, particularly when preparing a liquid formulation of a fusion protein such as a TACI-Fc, to prepare a formulation in which the protein is in a charged state to assist with solubility of the protein and avoid aggregation of the protein that can lead to presence of high molecular weight (HMW) species. Certain TACI-Fc proteins, however, have a relatively low isoelectric point (pI) compared to many other Fc-comprising fusion proteins, which may make a high concentration formulation more challenging as the formulation may require a relatively low pH. At a low pH, the risk of degradation and formation of low molecular weight (LMW) species increases, for example. In some embodiments, the TACI-Fc fusion protein has an isoelectric point (pI) of at or about 7.0 to about 7.8, at or about 7.0 to about 7.7, at or about 7.0 to about 7.6, at or about 7.0 to about 7.5, at or about 7.0 to about 7.3, at or about 7.1 to about 7.5, at or about 7.1 to about 7.3, or about 7.2. In spite of this relatively low pI, as described herein, it was possible to formulate the TACI-Fc fusion proteins at high concentrations and relatively low pH that yet remained stable with low concentrations of HMW and LMW species after storage.

In some embodiments, the pH of the pharmaceutical formulation is between about 4.3 and about 5.2, such as between about 4.3 and about 5.1, such as between about 4.4 and about 5.1, such as between about 4.7 and about 5.1, such as between about 4.3 and about 4.9, between about 4.3 and about 4.8, between about 4.3 and about 4.7, between about 4.3 and about 4.6, between about 4.4 and about 5.0, between about 4.4 and about 4.8, between about 4.4 and about 4.7, between about 4.45 and about 5.0, between about 4.45 and about 4.8, between about 4.45 and about 4.7, between about 4.5 and about 5.0, between about 4.5 and about 4.8, between about 4.5 and about 4.7, between about 4.55 and about 5.0, between about 4.55 and about 4.8, between about 4.55 and about 4.7, between about 4.6 and about 5.0, or between about 4.6 and about 4.8, between about 4.65 and about 5.0, or between about 4.65 and about 4.8, or between about 4.7 and 4.8. In some embodiments, the pH of the pharmaceutical formulation is at or about 4.3, 4.4, 4.5, 4.6, 4.7, 4.8, 4.9, 5.0, 5.1, or 5.2. In some embodiments, the pH of the pharmaceutical formulation is 4.7±0.2. In some embodiments, the pH of the pharmaceutical formulation is at or about 4.6. In some embodiments, the pH of the pharmaceutical formulation is at or about 4.65. In some embodiments, the pH of the pharmaceutical formulation is at or about 4.7. In some embodiments, the pH of the pharmaceutical formulation is at or about 4.75. In some embodiments, the pH of the pharmaceutical formulation is at or about 4.8.

The pharmaceutical formulations provided herein can comprise a buffering agent. In some embodiments, the pharmaceutical formulations comprise an acetate buffer. In some embodiments, the pharmaceutical composition can comprise an acetate buffer at a pH of between about pH 4.0-6.0. In some embodiments, the formulation buffer contains from about 1 mM to about 30 mM, from about 2 mM to about 20 mM, from about 3 mM to about 15 mM, from about 4 mM to about 20 mM, or from 5 mM to 15 mM, or from about 5 mM to about 10 mM, or from about 8 mM to about 25 mM, from about 10 mM to about 30 mM, from about 15 mM to about 30 mM, or from about 7 mM to about 25 mM acetate as a component of the acetate buffer. In some embodiments, the formulation buffer provided herein contains acetate at a concentration of from about 5 mM to about 30 mM. In some embodiments, the buffered solution contains acetate at a concentration of at or about 5 mM, at or about 6 mM, at or about 7 mM, at or about 8 mM, at or about 9 mM, at or about 10 mM, at or about 11 mM, at or about 12 mM, at or about 13 mM, at or about 14 mM, at or about 15 mM, at or about 16 mM, at or about 17 mM, at or about 18 mM, at or about 19 mM, at or about 20 mM, at or about 21 mM, at or about 22 mM, at or about 23 mM, at or about 24 mM, at or about 25 mM, at or about 26 mM, at or about 27 mM, at or about 28 mM, at or about 29 mM, or at or about 30 mM, or any value between any of the foregoing. In some embodiments, the buffered solution contains acetate at a concentration of at or about 5 mM. In some embodiments, the buffered solution contains acetate at a concentration of at or about 10 nM. In some embodiments, the buffered solution contains acetate at a concentration of at or about 12 mM. In some embodiments, the buffered solution contains acetate at a concentration of at or about 15 mM. In some embodiments, the buffered solution contains acetate at a concentration of at or about 20 mM. Exemplary pH ranges of an acetic acid (acetate) buffer and/or the final formulation can include pH ranges between about 4.3 and about 5.2, such as between about 4.3 and about 5.1, such as between about 4.4 and about 5.1, such as between about 4.7 and about 5.1, such as between about 4.3 and about 4.9, between about 4.3 and about 4.8, between about 4.3 and about 4.7, between about 4.3 and about 4.6, between about 4.4 and about 5.0, between about 4.4 and about 4.8, between about 4.4 and about 4.7, between about 4.45 and about 5.0, between about 4.45 and about 4.8, between about 4.45 and about 4.7, between about 4.5 and about 5.0, between about 4.5 and about 4.8, between about 4.5 and about 4.7, between about 4.55 and about 5.0, between about 4.55 and about 4.8, between about 4.55 and about 4.7, between about 4.6 and about 5.0, or between about 4.6 and about 4.8, between about 4.65 and about 5.0, or between about 4.65 and about 4.8, or between about 4.7 and 4.8. Accordingly, an acetic acid (acetate) buffer and/or the final formulation can be prepared to have a pH of about 4.3 and about 5.1, such as between about 4.3 and about 4.9, between about 4.3 and about 4.8, between about 4.3 and about 4.7, between about 4.3 and about 4.6, between about 4.4 and about 5.0, between about 4.4 and about 4.8, between about 4.4 and about 4.7, between about 4.45 and about 5.0, between about 4.45 and about 4.8, between about 4.45 and about 4.7, between about 4.5 and about 5.0, between about 4.5 and about 4.8, between about 4.5 and about 4.7, between about 4.55 and about 5.0, between about 4.55 and about 4.8, between about 4.55 and about 4.7, between about 4.6 and about 5.0, or between about 4.6 and about 4.8, between about 4.65 and about 5.0, or between about 4.65 and about 4.8, or between about 4.7 and 4.8. In some embodiments, the pH of the buffered solution is at or about 4.7. In some embodiments, the pH of the buffered solution is 4.7±0.2.

### C. Free Amino Acids

In some embodiments, the formulation comprises one or more tonicifying agents. In some embodiments, the tonicifying agent may comprise an amino acid and/or a salt. In some embodiments, the formulation has a tonicity of an isotonic tonicity, of at or about 250 to about 350 mOsm/Kg, or of at or about 250 mOsm/Kg to about 600 mOsm/Kg. In some embodiments, the formulation is an isotonic formulation.

Amino acids can be used for stabilizing proteins in a provided formulation as bulking agents, stabilizers, viscosity reducing agents, and antioxidants, as well as other standard uses. The formulations provided herein contain free amino acids, such as but not limited to, arginine and proline, and conservative amino acid substitutions of arginine or proline. In some embodiments the formulations provided herein contain a free amino acid arginine. Arginine in the formulations provided herein can act as a stabilizer to maintain protein (e.g., TACI-Fc) stability and integrity of during storage and transportation, reduce viscosity, and protect against denaturation and aggregation. In some embodiments the formulations provided herein contain a free amino acid proline. In particular embodiments where the formulations provided herein contain a free amino acid proline, the formulations also contain a salt, such as, for example, sodium chloride (NaCl). In some embodiments, the free amino acid is present in the formulation at a concentration of at or about 1% to about 4% or 2% to 5% (w/v).

In some embodiments, the formulation contains arginine as a free amino acid. In some embodiments, the formulation contains a salt form of arginine such as ArgHCl, ArgCl, Arg2SO4, ArgGlu, ArgAcetate, or others. Arginine Hydrochloride is a hydrochloride salt form of arginine. In some embodiments herein, the arginine is arginine hydrochloride (Arg-HCl). In embodiments herein, the arginine is L-arginine. In some embodiments, the concentration of arginine is expressed as a molar concentration. In some embodiments, the formulations contain arginine at a concentration of at or about 50 mM to about 200 mM. In some embodiments, the formulations contain arginine at a concentration of at or about 50 mM to about 200 mM, 70 mM to about 190 mM; at or about 135 mM to about 165 mM; at or about 145 M to about 150 mM; at or about 140 mM, at or about 145 mM, at or about 150 mM, or at or about 155 mM. In some embodiments, the formulations contain at or about 135 mM to about 165 mM arginine. In some embodiments, the formulations contain at or about 140 mM to about 150 mM arginine. In embodiments herein, the concentration of arginine, such as an arginine salt such as Arg-HCl, in the formulation is expressed as weight/volume (w/v). In some embodiments, provided formulations contain arginine at a concentration of about 1% to about 5% (w/v). In some embodiments, provided formulations contain arginine at a concentration of about 1.5% to about 4% (w/v). In some embodiments, provided formulations contain arginine at a concentration of about 2.7% to about 3.5% (w/v). In some embodiments, provided formulations contain arginine at a concentration of at or about 1% (w/v), at or about 1.25% (w/v), at or about 1.5% (w/v), at or about 2% (w/v), at or about 2.5% (w/v), at or about 3% (w/v), at or about 3.1% (w/v), at or about 3.2% (w/v), at or about 3.3% (w/v), at or about 3.5% (w/v), at or about 3.7% v, at or about 4% (w/v), or at or about 5% (w/v), or any value between any of the foregoing. In some embodiments, provided formulations contain arginine at a concentration of about or about 2% (w/v). In some embodiments, provided formulations contain arginine at a concentration of at or about 3% (w/v). In some embodiments, the provided formulations contain arginine at a concentration of at or about 4% (w/v). In any of the provided embodiments, the arginine is arginine hydrochloride (Arg-HCl). For example, in some embodiments the provided formulations contain arginine, such as Arg-HCl, at a concentration of at or about 3.1% (w/v).

In some embodiments, the formulation contains proline as a free amino acid. In embodiments herein, the proline is L-proline. In some embodiments herein, the percentage of proline, such as L-proline in the formulation is expressed as weight/volume (w/v). In some embodiments, provided formulations contain proline at a concentration of about 1.0% (w/v) to about 5% (w/v). In some embodiments, provided formulations contain proline at a concentration of about 1.0% (w/v) to about 4% (w/v). In some embodiments, provided formulations contain proline at a concentration of at or about 1% (w/v), at or about 1.25% (w/v), at or about 1.5% (w/v), at or about 2% (w/v), at or about 2.5% (w/v), at or about 3% (w/v), at or about 4% (w/v), or at or about 5% (w/v), or any value between any of the foregoing. In some embodiments, provided formulations contain proline at a concentration of at or about 2% (w/v). In some embodiments, provided formulations contain proline at a concentration of at or about 3% (w/v). In some embodiments, provided formulations contain proline at a concentration of at or about 4% (w/v).

### D. Salt

In some embodiments, the formulation contains one or more salts. In some embodiments, the formulation contains a salt such as sodium chloride (NaCl), sodium thiocyanate (NaSCN), magnesium chloride (MgCl), magnesium sulfate (MgSO₄), ammonium thiocyanate (NH₄SCN), ammonium chloride (NH₄Cl), calcium chloride (CaCl₂)), calcium sulfate (CaSO₄), zinc chloride (ZnCl₂) and the like, or combinations thereof. In some embodiments the formulation contains sodium chloride (NaCl).

In some embodiments the amount of salt in the formulation is at or about 20 mM to at or about 150 mM, is at or about 30 mM to at or about 130 mM, is at or about 30 mM to at or about 120 mM, is at or about 30 mM to at or about 100 mM, is at or about 30 mM to at or about 90 mM, is at or about 30 mM to at or about 80 mM, is at or about 30 mM to at or about 70 mM, is at or about 30 mM to at or about 60 mM, is at or about 30 mM to at or about 50 mM, is at or about 40 mM to at or about 130 mM, is at or about 40 mM to at or about 120 mM, is at or about 40 mM to at or about 100 mM, is at or about 40 mM to at or about 90 mM, is at or about 40 mM to at or about 80 mM, is at or about 40 mM to at or about 70 mM, is at or about 40 mM to at or about 60 mM, is at or about 40 mM to at or about 50 mM, is at or about 50 mM to at or about 130 mM, is at or about 50 mM to at or about 120 mM, is at or about 50 mM to at or about 100 mM, is at or about 50 mM to at or about 90 mM, is at or about 50 mM to at or about 80 mM, is at or about 50 mM to at or about 70 mM, is at or about 50 mM to at or about 60 mM, is at or about 50 mM to at or about 130 mM, is at or about 50 mM to at or about 120 mM, is at or about 60 mM to at or about 100 mM, is at or about 60 mM to at or about 90 mM, is at or about 60 mM to at or about 80 mM, or is at or about 60 mM to at or about 70 mM. In some embodiments the amount of salt in the formulation is at or about 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, or 150 mM, or a value between any of the foregoing. In any of the embodiments herein, the salt is NaCl.

In some embodiments the amount of NaCl in the formulation is at or about 30 mM to at or about 150 mM. In some embodiments the amount of NaCl in the formulation is at or about 40 mM to at or about 100 mM. In some embodiments, the amount of salt, such as NaCl, is at or about 50 mM. In some embodiments, the amount of salt, such as NaCl, is at or about 40 mM.

In embodiments, a salt or salts is added to pharmaceutical formulations provided herein in the presence of an amino acid in the formulation that is proline. In embodiments, a salt or salts is added to pharmaceutical formulations provided herein in the presence of an amino acid in the formulation that is L-proline.

### E. Surfactant

Provided formulations may also further comprise surfactants. Protein molecules may be susceptible to adsorption on surfaces and to denaturation and consequent aggregation at air-liquid, solid-liquid, and liquid-liquid interfaces. In some cases, the effects may be exacerbated by physical agitation, such as that generated during the shipping and handling of a product. Surfactants may be used to prevent, minimize, or reduce surface adsorption. A surfactant for inclusion in a formulation can be chosen, for example, to enhance or promote retention in stability of the protein molecule by preventing or reducing aggregation and/or adsorption. Sorbitan fatty acid esters such as the polysorbates are surfactants exhibiting a wide range of hydrophilic and emulsifying characteristics. They can be used individually or in combination with other surfactants to cover a wide range of stabilization needs. Such characteristics can be suitable for use with active protein agents because they can be tailored to cover the wide range of hydrophobic and hydrophilic characteristics of biopharmaceuticals. In particular embodiments, no surfactant is included in the formulation. In such examples, the other components of the formulation are modified to achieve a stable formulation.

Examples of pharmaceutically acceptable surfactants for use in pharmaceutical formulations include, but are not limited to, polyoxyethylen-sorbitan fatty acid esters (Polysorbate, e.g., Tween®), polyoxyethylene alkyl ethers (Brij), alkylphenylpolyoxyethylene ethers (Triton X), polyoxyethylene-polyoxypropylene copolymers (Poloxamer, Pluronic), sodium dodecyl sulphate (SDS) and n-dodecyl-o-D-maltoside. Exemplary polyoxyethylene-sorbitan fatty acid esters are polysorbate 20 (polyoxyethylene sorbitan monolaureate e.g.,) and polysorbate 80 (polyoxyethylene sorbitan monooleate e.g., sold under the trademark Tween® 80). Exemplary polyethylene-polypropylene copolymers are those sold under the names Pluronic® F68 or Poloxamer 188. Exemplary polyoxyethylene alkyl ethers are those sold under the trademark Brij™. Exemplary alkylphenylpolyoxyethylene ethers are sold under the tradename Triton X, such as p-tert-octylphenoxy polyethoxyethanol (sold under the tradename Triton X-100™). Exemplary surfactants for use herein are polyoxyethylen-sorbitan fatty acid esters, preferably polysorbate 20 or polysorbate 80. Another exemplary surfactant for inclusion in the formulations herein is Poloxamer 188.

In some embodiments, the surfactant comprised within the formulation is a non-ionic surfactant. Non-ionic surfactants such as polysorbates, including polysorbate 80 and poloxamers, including poloxamer 188 can stabilize the antibody during processing and storage by reducing interfacial interaction and prevent protein from adsorption. Useful surfactants in pharmaceutical formulations include, but are not limited to, non-ionic surfactants, such as poloxamers, polyol esters, and polyoxyethylene esters. For example, pharmaceutical formulations generally include non-ionic surfactants such as poloxamers such as poloxamer 188; and polyoxyethylene esters such as polysorbate 20 or polysorbate 80. In other embodiments, the formulation does not comprise a polysorbate surfactant, but may, for example, comprise a poloxamer surfactant such as poloxamer 188.

In embodiments herein, the amount of surfactant in the formulation is less than 1% (w/v). In this regard, surfactant concentrations in the formulations here generally can be used at ranges at or about 0.001-1.0% (w/v), such as at or about 0.002-1.0% (w/v), such as at or about 0.001 to about 0.20% (w/v), such as at or about 0.01 to about 0.20% (w/v), at or about 0.01 to about 0.175% (w/v), at or about 0.01 to about 0.15% (w/v), at or about 0.01-0.125% (w/v), at or about 0.01 to about 0.1% (w/v), at or about 0.01 to about 0.075% (w/v), at or about 0.01 to about 0.05% (w/v), at or about 0.01 to about 0.025% (w/v), at or about 0.01 to about 0.02% (w/v), at or about 0.015 to about 0.175% (w/v), at or about 0.015 to about 0.15% (w/v), at or about 0.015 to about 0.125% (w/v), at or about 0.015 to about 0.1% (w/v), at or about 0.015 to about 0.075% (w/v), at or about 0.015 to about 0.05% (w/v), at or about 0.015 to about 0.025% (w/v), at or about 0.015 to about 0.02% (w/v), at or about 0.02 to about 0.175% (w/v), at or about 0.02 to about 0.15% (w/v), at or about 0.02 to about 0.125% (w/v), at or about 0.02 to about 0.1% (w/v), at or about 0.02 to about 0.075% (w/v), at or about 0.02 to about 0.05% (w/v), at or about 0.02 to about 0.025% (w/v), at or about 0.04 to about 0.175% (w/v), at or about 0.04 to about 0.15% (w/v), at or about 0.04 to about 0.16% (w/v), at or about 0.04 to about 0.17% (w/v), at or about 0.04 to about 0.125% (w/v), at or about 0.04 to about 0.1% (w/v), at or about 0.04 to about 0.075% (w/v), or at or about 0.04 to about 0.05% (w/v). Surfactant concentrations and/or amounts less than, greater than or in between these ranges also can also be used. Accordingly, a formulation can be produced that contains essentially any desired concentration or amount of one or more surfactants including, for example, at or about 0.010% (w/v), 0.015% (w/v), 0.02% (w/v), 0.025% (w/v), 0.03% (w/v), 0.04% (w/v), 0.05% (w/v), 0.06% (w/v), 0.07% (w/v), 0.08% (w/v), 0.09% (w/v), 0.10% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.20% (w/v), or any value between any of the foregoing. In some embodiments, the formulation contains about 0.002% to about 1.0% (w/v) of surfactant. In some embodiments, the formulation contains about 0.015% to about 0.1% (w/v) of surfactant. In some embodiments, the formulation contains about 0.04% to about 0.16% (w/v) of surfactant. In particular embodiments, the formulation contains at or about 0.015% (w/v) of surfactant. In particular embodiments, the formulation contains at or about 0.1% (w/v) of surfactant.

In some embodiments, the surfactant is polysorbate 80. In some embodiments, polysorbate 80 is at a concentration in the formulation of at or about 0.01 to about 0.20% (w/v), such as at or about 0.01 to about 0.175% (w/v), at or about 0.01 to about 0.15% (w/v), at or about 0.01 to about 0.125% (w/v), at or about 0.01 to about 0.1% (w/v), at or about 0.01 to about 0.075% (w/v), at or about 0.01 to about 0.05% (w/v), at or about 0.01 to about 0.025% (w/v), at or about 0.01 to about 0.02% (w/v), at or about 0.015 to about 0.175% (w/v), at or about 0.015 to about 0.15% (w/v), at or about 0.015 to about 0.125% (w/v), at or about 0.015 to about 0.1% (w/v), at or about 0.015 to about 0.075% (w/v), at or about 0.015 to about 0.05% (w/v), at or about 0.015 to about 0.025% (w/v), at or about 0.015 to about 0.02% (w/v), at or about 0.02 to about 0.175% (w/v), at or about 0.02 to about 0.15% (w/v), at or about 0.02 to about 0.125% (w/v), at or about 0.02 to about 0.1% (w/v), at or about 0.02 to about 0.075% (w/v), at or about 0.02 to about 0.05% (w/v), at or about 0.02 to about 0.025% (w/v), at or about 0.04 to about 0.175% (w/v), at or about 0.04 to about 0.15% (w/v), at or about 0.04 to about 0.16% (w/v), at or about 0.04 to about 0.17% (w/v), at or about 0.04 to about 0.125% (w/v), at or about 0.04 to about 0.1% (w/v), at or about 0.04 to about 0.075% (w/v), or at or about 0.04 to about 0.05% (w/v). In some embodiments, polysorbate 80 is present at a concentration of 0.001% (w/v), 0.002% (w/v), 0.003% (w/v), 0.004% (w/v), 0.005% (w/v), 0.006% (w/v), 0.007% (w/v), 0.008% (w/v), 0.009% (w/v), 0.010% (w/v), 0.015% (w/v), 0.02% (w/v), 0.025% (w/v), 0.03% (w/v), 0.04% (w/v), 0.05% (w/v), 0.06% (w/v), 0.07% (w/v), 0.08% (w/v), 0.09% (w/v) or 0.10% (w/v), or any value between any of the foregoing. In some embodiments, the concentration of polysorbate 80 is at or about 0.010% (w/v). In some embodiments, the concentration of polysorbate 80 is at or about 0.015% (w/v). In some embodiments, the concentration of polysorbate 80 is at or about 0.02% (w/v).

In some embodiments, the surfactant is poloxamer 188. In some embodiments, poloxamer 188 is at a concentration in the formulation of at or about 0.01 to about 0.20% (w/v), such as at or about 0.05 to about 0.20% (w/v), such as at or about 0.01 to about 0.175% (w/v), at or about 0.01 to about 0.15% (w/v), at or about 0.01 to about 0.125% (w/v), at or about 0.01 to about 0.1% (w/v), at or about 0.01 to about 0.075% (w/v), at or about 0.01 to about 0.05% (w/v), at or about 0.01 to about 0.025% (w/v), at or about 0.01 to about 0.02% (w/v), at or about 0.015 to about 0.175% (w/v), at or about 0.015 to about 0.15% (w/v), at or about 0.015 to about 0.125% (w/v), at or about 0.015 to about 0.1% (w/v), at or about 0.015 to about 0.075% (w/v), at or about 0.015 to about 0.05% (w/v), at or about 0.015 to about 0.025% (w/v), at or about 0.015 to about 0.02% (w/v), at or about 0.02 to about 0.175% (w/v), at or about 0.02 to about 0.15% (w/v), at or about 0.02 to about 0.125% (w/v), at or about 0.02 to about 0.1% (w/v), at or about 0.02 to about 0.075% (w/v), at or about 0.02 to about 0.05% (w/v), at or about 0.02 to about 0.025% (w/v), at or about 0.04 to about 0.175% (w/v), at or about 0.04 to about 0.15% (w/v), at or about 0.04 to about 0.16% (w/v), at or about 0.04 to about 0.17% (w/v), at or about 0.04 to about 0.125% (w/v), at or about 0.04 to about 0.1% (w/v), at or about 0.04 to about 0.075% (w/v), or at or about 0.04 to about 0.05% (w/v). In some embodiments, poloxamer 188 is present at a concentration of 0.010% (w/v), 0.015% (w/v), 0.02% (w/v), 0.025% (w/v), 0.03% (w/v), 0.04% (w/v), 0.05% (w/v), 0.06% (w/v), 0.07% (w/v), 0.08% (w/v), 0.09% (w/v), 0.10% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.20% (w/v), or any value between any of the foregoing. In some embodiments, the concentration of poloxamer 188 is at or about 0.10% (w/v). In some embodiments, the concentration of poloxamer 188 is at or about 0.15% (w/v). In some embodiments, the concentration of poloxamer 188 is at or about 0.2% (w/v).

### F. Other Excipients

Including and in addition to the excipients listed above, the pharmaceutical formulations provided herein can comprise one or more additional pharmaceutically acceptable excipient(s). In certain embodiments, the pharmaceutical formulation may contain formulation materials for modifying, maintaining or preserving, for example, the pH, osmolarity, viscosity, clarity, color, isotonicity, sterility, stability, rate of dissolution or release, adsorption or penetration of the formulation. Such formulations may further comprise, for example, antimicrobials; antioxidants (such as ascorbic acid, sodium sulfite or sodium hydrogen-sulfite); additional buffers (such as acetate, borate, bicarbonate, Tris-HCl, citrates, adipate, succinate, histidine, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; preservatives (such as benzalkonium chloride, benzoic acid, salicylic acid, thimerosal, phenethyl alcohol, methylparaben, ethylparaben, propylparaben, chlorhexidine, sorbic acid or hydrogen peroxide); solvents (such as glycerin, propylene glycol or polyethylene glycol); or sugar alcohols (such as mannitol or sorbitol). See, REMINGTON'S PHARMACEUTICAL SCIENCES, 18” Edition, (A. R. Genrmo, ed.), 1990, Mack Publishing Company.

### G. Exemplary TACI-Fc Formulations

In some embodiments, a provided TACI-Fc fusion protein is provided in a formulation that comprises water and a buffering agent that provides a formulation pH of at or about 4.3 to about 5.5, a tonicifying agent having a positive charge at the formulation pH, a non-ionic surfactant, and a TACI-Fc fusion protein at a concentration of at or about 100 mg/mL to about 200 mg/mL. In some embodiments, the formulation is a liquid formulation. In some embodiments, the liquid formulation is not lyophilized before administration and has not been lyophilized. In some embodiments, the concentration or amount of any of the above components can be as described in any of Sections II.A-F or as described herein.

In some embodiments, a provided TACI-Fc fusion protein is provided in a formulation that comprises at or about 50 mM to about 200 mM of a tonicifying agent, wherein the tonicifying agent comprises arginine, an acetate buffer, and at or about 0.002% to about 1.0% (w/v) of a non-ionic surfactant; wherein the formulation comprises a TACI-Fc fusion protein concentration of at or about 140 mg/mL to about 200 mg/mL and a formulation pH of at or about 4.4 to about 5.1. In some embodiments, the formulation is a liquid formulation. In some embodiments, the liquid formulation is not lyophilized before administration and has not been lyophilized. In some embodiments, the concentration or amount of any of the above components can be as described in any of Sections II.A-F or as described herein.

In some embodiments, a provided TACI-Fc fusion protein is provided in a formulation that comprises at or about 135 mM to about 165 mM arginine or at or about 140 mM to about 150 mM arginine or at or about 1.5% to about 4% (w/v) arginine hydrochloride (Arg-HCl), about 5 mM to about 30 mM of an acetate, and about 0.01% to about 0.2% (w/v) of a non-ionic surfactant such as poloxamer 188, in which the formulation pH is at or about 4.6 to about 4.8. In some embodiments, the concentration of the TACI-Fc fusion protein in the composition is between about 150 mg/mL and about 200 mg/mL. In some embodiments, the concentration of the TACI-Fc fusion protein in the composition is between about 160 mg/mL and about 190 mg/mL. In some embodiments, the formulation is a liquid formulation. In some embodiments, the liquid formulation is not lyophilized before administration and has not been lyophilized. In some embodiments, the concentration or amount of any of the above components can be as described in any of Sections II.A-F or as described herein.

In some embodiments, the TACI-Fc fusion protein, such as any described herein, is formulated in a buffered solution containing 10 mM sodium acetate, at or about 135 mM to about 165 mM arginine or at or about 140 mM to about 150 mM arginine or at or about 3.1% arginine HCl w/v, 0.1% poloxamer 188 w/v at a pH of about 4.7. In some embodiments the TACI-Fc fusion protein, such as any described herein, is formulated in a buffered solution containing 10 mM sodium acetate, at or about 135 mM to about 165 mM arginine or at or about 140 mM to about 150 mM arginine or at or about 3.1% arginine HCl w/v, 0.1% poloxamer 188 w/v at a pH of at or about 4.6 to about 4.8 such as about 4.7, wherein the TACI-Fc fusion protein is a homodimer of two polypeptides of the formula TACI-linker-Fc.

In some embodiments, the TACI-Fc fusion protein, such as any described herein, is formulated in a buffered solution containing 10 mM sodium acetate, at or about 135 mM to about 165 mM arginine or at or about 140 mM to about 150 mM arginine or 3.1% arginine HCl w/v, 0.1% poloxamer 188 w/v at a pH of about 4.6. In some embodiments the TACI-Fc fusion protein, such as any described herein, is formulated in a buffered solution containing 10 mM sodium acetate, 3.1% arginine HCl w/v, 0.1% poloxamer 188 w/v at a pH of about 4.6, wherein the TACI-Fc fusion protein is a homodimer of two polypeptides of the formula TACI-linker-Fc.

In some embodiments, the TACI-Fc fusion protein is provided at about 150 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 210 mg/mL, about 220 mg/mL, or about 230 mg/mL, or a value between any of the foregoing. In some embodiments, the TACI-Fc fusion protein is provided at 160 mg/mL as a liquid for injection (e.g. IV or SC). In some embodiments, the TACI-Fc fusion protein is provided at 175 mg/mL as a liquid for injection (e.g. IV or SC). In some embodiments, the TACI-Fc fusion protein is provided in a volume of at or about 0.46 mL (e.g. 80 mg). In some embodiments, the TACI-Fc fusion protein is provided in a volume of at or about 1.37 mL (e.g. 240 mg). In some embodiments, the TACI-Fc fusion protein volume is provided in an injection device (e.g., autoinjector).

In some embodiments, a provided TACI-Fc fusion protein is provided in a formulation that comprises about 1.0% to about 4% (w/v) proline (e.g., L-proline), about 5 mM to about 30 mM of an acetate; about 0.002% to about 0.2% (w/v) of non-ionic surfactant; and a salt, in which the formulation pH is at or about 4.4 to about 5.2. In some embodiments, the salt is sodium chloride (NaCl). In some embodiments, the formulation comprises between about 40 mM and 100 mM NaCl, such as about 50 mM. In some embodiments, the concentration of the TACI-Fc fusion protein in the composition is between about 140 mg/mL and about 200 mg/mL. In some embodiments, the formulation is a liquid formulation. In some embodiments, the liquid formulation is not lyophilized before administration and has not been lyophilized. In some embodiments, the concentration or amount of any of the above components can be as described in any of Sections II.A-F or as described herein.

In some embodiments, the TACI-Fc fusion protein, such as any described herein, is provided in a formulation that comprises about 1.0% to about 4% (w/v) proline (e.g., L-proline); an acetate buffer such as at or about 5 mM to about 30 mM of an acetate; and a salt, in which the formulation pH is at or about 4.4 to about 5.2. In some embodiments, the salt is sodium chloride (NaCl). In some embodiments, the formulation comprises between about 40 mM and 100 mM NaCl, such as about 50 mM. In some embodiments, the concentration of the TACI-Fc fusion protein in the composition is between about 140 mg/mL and about 200 mg/mL. In some embodiments, the formulation further comprises a non-ionic surfactant such as polysorbate 80 or poloxamer 188. In some embodiments, the formulation is a liquid formulation. In some embodiments, the liquid formulation is not lyophilized before administration and has not been lyophilized. In some embodiments, the concentration or amount of any of the above components can be as described in any of Sections II.A-F or as described herein.

In some embodiments, the TACI-Fc fusion protein, such as any described herein, is formulated in a buffered solution containing about 2.0% or about 3.0% (w/v) proline, an acetate buffer such as at or about 10 mM sodium acetate, at or about 0.05% to about 0.2%, such as at or about 0.1% poloxamer 188 (w/v), and at or about 50 mM sodium chloride, at a pH of about 4.7. In other embodiments, the TACI-Fc fusion protein, such as any described herein, is formulated in a buffered solution containing about 2.0% or about 3.0% (w/v) proline, an acetate buffer such as at or about 10 mM sodium acetate, at or about 0.01% to about 0.04% polysorbate 80 (w/v), and at or about 50 mM sodium chloride, at a pH of about 5.2. In some embodiments, the TACI-Fc fusion protein is provided at about 150 mg/mL, about 160 mg/mL, about 165 mg/mL, about 170 mg/mL, about 175 mg/mL, about 180 mg/mL, about 185 mg/mL, about 190 mg/mL, about 195 mg/mL, 200 mg/mL, or a value between any of the foregoing. In some embodiments, the TACI-Fc fusion protein is provided at 160 mg/mL as a liquid for injection (e.g. IV or SC). In some embodiments, the TACI-Fc fusion protein is provided at 175 mg/mL as a liquid for injection (e.g. IV or SC). In some embodiments, the TACI-Fc fusion protein is provided in a volume of at or about 0.46 mL (e.g. 80 mg). In some embodiments, the TACI-Fc fusion protein is provided in a volume of at or about 1.37 mL (e.g. 240 mg). In some embodiments, the TACI-Fc fusion protein volume is provided in an injection device (e.g., autoinjector).

In some embodiments, the TACI-Fc fusion protein comprises (i) a TACI polypeptide sequence comprising the amino acid sequence of SEQ ID NO: 26; and (ii) an immunoglobulin Fc region. In some embodiments, the immunoglobulin Fc region comprises the amino acid sequence of any one of SEQ ID NOS: 73, 75, 83, 136, 175, 176, or 221. In some embodiments, the TACI-Fc fusion protein comprises the amino acid sequence of SEQ ID NO: 168 or SEQ ID NO: 167. In some embodiments, the TACI-Fc fusion protein is a homodimer comprising two identical copies of the TACI-Fc fusion protein. In some embodiments, the TACI-Fc fusion protein is a homodimer comprising two identical copies of the TACI-Fc fusion protein set forth in SEQ ID NO:168 linked by a covalent disulfide bond. In some embodiments, the TACI-Fc fusion protein is a homodimer comprising two identical copies of the TACI-Fc fusion protein set forth in SEQ ID NO:167 linked by a covalent disulfide bond. In some embodiments, the TACI-Fc fusion protein comprises a variant TACI polypeptide, and/or the TACI-Fc fusion protein has an isoelectric point (pI) of at or about 7.0 to about 7.8, at or about 7.0 to about 7.7, at or about 7.0 to about 7.6, at or about 7.0 to about 7.5, at or about 7.0 to about 7.3, at or about 7.1 to about 7.5, at or about 7.1 to about 7.3, or about 7.2.

### III. PROPERTIES AND METHODS FOR ASSESSING THE PROPERTIES OF THE PHARMACEUTICAL FORMULATIONS AND TACI-FC PROTEIN IN THE FORMULATIONS

The stability of the TACI-Fc protein in the formulations herein can be assessed by various methods known in the art.

In some embodiments, the pharmaceutical formulations, such as any of the provided formulations, are stable at or about 2-8° C., such as at or about 5° C., for up to 36 months or more, such as for up to 12 months or more, such as up to 24 months, such as up to 36 months or more. In some embodiments, the pharmaceutical formulation is stored at or about 5° C. In some embodiments, the pharmaceutical formulations, such as any of the provided formulations, are stable at or about −20° C. for up to 36 months or more, such as for up to 12 months or more, such as up to 24 months, such as up to 36 months or more. In some embodiments, the pharmaceutical formulation is stored at or about −20° C. In some embodiments, the pharmaceutical formulations, such as any of the provided formulations, are stable at or about −70° C. for up to 36 months or more, such as for up to 12 months or more, such as up to 24 months, such as up to 36 months or more. In some embodiments, the pharmaceutical formulation is stored at or about −70° C. In some embodiments, the pharmaceutical formulations, such as any of the provided formulations, are stable at or about 25° C., for up to 6 months or more, such as for up to 2 months or more, such as up to 3 months, such as up to 6 months or more. In some embodiments, the pharmaceutical formulation is stored at or about 25° C. In some embodiments, the storage is under conditions in which the formulation of the TACI-Fc fusion protein is protected from light. In some conditions the formulation of the TACI-Fc fusion protein is stored in an injection device at refrigerated temperatures, such as, for example, 2-8° C., such as at or about 5° C.

In some embodiments, the pharmaceutical formulation is sterile. Sterilization may be accomplished by filtration through sterile filtration membranes. In some embodiments, the liquid formulation provided herein is lyophilized. Where the formulation is lyophilized, sterilization of the lyophilized product may be conducted either prior to lyophilization or following lyophilization and reconstitution. The formulation for parenteral administration may be stored in lyophilized form or in liquid form in solution. In addition, parenteral formulations generally are placed into a container having a sterile access port, for example, an intravenous solution bag or vial having a stopper pierceable by a hypodermic injection needle. In some embodiments herein, the sterile pharmaceutical formulation is stored in an injection device. In some embodiments herein, the sterile pharmaceutical formulation is stored in a syringe. In some embodiments herein, the sterile pharmaceutical formulation is stored in an autoinjector.

Once the pharmaceutical formulation has been formulated, it may be stored in sterile vials as a solution, suspension, gel, emulsion, solid, crystal, or as a lyophilized powder. Such formulations may be stored either in a ready-to-use form (e.g., in a liquid form that can be directly administered) or in a form (e.g., lyophilized) that is reconstituted prior to administration. In embodiments herein, the pharmaceutical formulation is stored in sterile vials as a liquid solution. In some such cases, the formulation is not lyophilized prior to administration and has not been lyophilized. In other embodiments the liquid formulation provided herein is lyophilized. Also provided herein are lyophilized forms of the liquid formulations provided herein. Also provided herein are kits for producing a single-dose administration unit. In some aspects, the kits may each contain both a first container having a dried protein and a second container having an aqueous formulation. In certain embodiments kits containing single and multi-chambered pre-filled syringes (e.g., liquid syringes and lyosyringes) are provided. In embodiments herein, the TACI-Fc formulation is prepared as a liquid formulation. In embodiments herein, the TACI-Fc formulation is prepared as a liquid formulation and stored in a sterile injection device. In particular embodiments, the TACI-Fc formulation is prepared as a liquid formulation. In embodiments herein, the TACI-Fc formulation is prepared as a liquid formulation and stored in a pre-filled syringe within an autoinjector.

Provided herein are any of such formulations contained in a container such as a vial. In particular aspects, the container, such as vial, is sterile. The container may be any biocompatible container, such as a glass or plastic container. In some embodiments, the container is a glass vial. In some embodiments, the vial is a Type I borosilicate glass vial. In some embodiments, the container is an ISO2R glass vial. In some embodiments, the container is an ISO6R glass vial. Provided herein are any of such formulations contained in a container such as an injection device. In some aspects, the injection device is a syringe. In some aspects, the injection device is an autoinjector. In particular aspects, the container, such as autoinjector, is sterile. Provided herein are any of such formulations contained in a container such as an autoinjector. In particular aspects, the container, such as autoinjector, is sterile. In some aspects the injection device is for subcutaneous administration of the formulation containing the TACI-Fc fusion protein.

### A. Viscosity of the TACI-Fc Formulation

Highly viscous pharmaceutical formulations can be difficult to administer. Concentrated protein formulations in liquid form can exhibit high viscosities, which can prevent them from flowing easily through a syringe needle. The viscosity of a pharmaceutical formulation can affect the syringeability and/or injectability of the formulation. Syringeability is the ease with which a formulation is removed from a vial to a syringe. Increased viscosity of a solution, such as, for example, a pharmaceutical formulation, can decrease syringeability compared to a less viscous solution. Injectability relates to the ease of administration of a solution such as, for example, a pharmaceutical formulation, from an injectable device, such as a syringe. Injectability can encompass the ease with which a solution flows from a syringe. Increased viscosity of a solution, such as, for example, a pharmaceutical formulation, can decrease injectability compared to a less viscous solution. A more viscous solution can clog a needle making the solution difficult to expel and administer. The size of the needle also can affect the injectability of a viscous solution as a more viscous solution can be more difficult to expel through a smaller diameter syringe needle and can require more force to expel. A less viscous solution can be more easily injected into a subject.

In some embodiments herein, the viscosity of the formulation is suitable for administration via an injectable device. In some embodiments herein, the viscosity of the formulation is suitable for administration via an autoinjector. In embodiments herein, the viscosity of the formulation comprising a TACI-Fc fusion protein is less than about 15 cP.

High concentration protein formulations can result in issues with the administration when these formulations are stored at refrigerated temperatures (e.g., 2°-8° C.), as incubation at these low temperatures can cause the viscosity of the solution to increase (see e.g, Zhang et al., AAPS PharmSciTech 19, 1520-1528 (2018)). Storage at low temperatures can increase viscosity of the formulation, decreasing the syringeability and/or injectability of the formulation. The temperature of the formulation at the time of measurement also can impact viscosity. For example, a formulation at a refrigerated temperature (e.g., 2°-8° C.) can be more viscous than the same formulation at a higher temperature. In embodiments herein, the formulations comprising a TACI-Fc fusion protein retain a viscosity suitable for subcutaneous administration after prolonged storage at decreased or increased storage time.

In some embodiments the viscosity of the TACI-Fc formulation is assessed by methods known in the art. Various assays are known and include, but are not limited to, a cone and plate technique. In some embodiments the plate and cone technique includes a Peltier element set at a defined temperature, such as 5° C. to 25° C. as described herein. In other examples, a viscometer, such as a glass capillary viscometer, tuning fork vibration viscometer, or rotational viscometer, rheometer, viscosity cup consistometer is used to assess viscosity. Typically, a well-defined shear stress gradient is applied to the liquid formulation and the resulting shear rate is measured. The viscosity is the ratio of the shear stress to the shear rate.

In some embodiments, the TACI-Fc formulations described herein are characterized by low viscosity. The viscosity of the TACI-Fc formulations is sufficient for administration, such as subcutaneous or intravenous administration. In some embodiments, the viscosity of the formulation ranges from about 2 cP to about 20 cP, about 2 to about 15 cP, about 3 to about 15 cP, about 2 to about 10 cP, 4 cP to about 20 cP, about 4 to about 15 cP, about 4 cP to about 10 cP, about 4 cP to about 8 cP, about 4 to about 6 cP, about 5 to about 20 cP, about 5 cP to about 15 cP, about 5 cP to about 10 cP, at 5° C. to 25° C. For example, in some embodiments, the formulation has a viscosity of about 3 to about 15 cP, about 5 cP to about 7 cP, such as about 5 cP, 5.5 cP, 6.0 cP, 6.5 cP, 6.6 cP, 6.7 cP, 6.8 cP, 6.9 cP, or 7 cP, or a value between any of the forgoing at about 25° C. In some embodiments, the formulation has a viscosity of about 7 cP at about 25° C. In other embodiments, the formulation has a viscosity of about 5 cP to about 15 cP, such as about 5 cP, 5.5 cP, 6.0 cP, 6.5 cP, 7 cP, 7.5 cP, 8 cP, 8.5 cP, 9 cP, 9.5 cP, 10 cP, 10.5 cP, 11 cP, 12 cP, 13 cP, 14 cP, 15 cP, or a value between any of the forgoing at about 20° C. In some embodiments, the formulation has a viscosity of about 8 cP at about 20° C. In other embodiments, the formulation has a viscosity of about 5 cP to about 15 cP, such as about 5 cP, 5.5 cP, 6.0 cP, 6.5 cP, 7 cP, 8 cP, 9 cP, 9.5 cP, 10 cP, 10.5 cP, 11 cP, 12 cP, 13 cP, 14 cP, 15 cP, or a value between any of the forgoing at about 15° C. In some embodiments, the formulation has a viscosity of about 10 cP at about 15° C. For example, in some embodiments, the formulation has a viscosity of about 10 cP to about 20 cP, such as about 11 cP, 12 cP, 13 cP, 14 cP, 15 cP, 16 cP, 17 cP, 18 cP, 19 cP, or 20 cP or a value between any of the forgoing at about 5° C. In some embodiments, the formulation has a viscosity of about 15 cP at about 5° C. In embodiments herein, the viscosity is assessed using a cone and plate Rheometer. In some embodiments, for example, the formulation is a liquid formulation having a viscosity of at or about 3 cP to about 15 cP, 4 cP to about 20 cP, about 4 to about 15 cP, about 4 cP to about 10 cP, about 4 cP to about 8 cP, about 4 to about 6 cP, about 5 to about 20 cP, about 5 cP to about 15 cP, about 5 cP to about 10 cP, at about 25° C. as assessed using a rheometer or a cone and plate viscometer.

In some embodiments, the viscosity of the TACI-Fc formulation is maintained after incubation at refrigerated (e.g., 2-8° C.) or elevated (30° C. or 40° C.) temperatures. In some embodiments, the viscosity of the TACI-Fc formulation is maintained after storage for greater than 12 weeks at refrigerated temperatures (e.g., 2-8° C.) or at 25° C., 30° C. or 40° C. In some embodiments the viscosity of the TACI-Fc formulation increases by less than at or about 1%, at or about 2%, at or about 3%, at or about 4%, or at or about 5%, at or about 6%, at or about 7%, at or about 8%, at or about 9%, at or about 10%, at or about 11%, at or about 12%, at or about 13%, at or about 14%, at or about 15%, at or about 16%, at or about 17%, at or about 18%, at or about 19%, at or about 20%, at or about 21%, at or about 22%, at or about 23%, at or about 24%, at or about 25%, at or about 26%, at or about 27%, at or about 28%, at or about 29%, at or about 30%, at or about 31%, at or about 32%, at or about 33%, at or about 34%, at or about 35%, at or about 36%, at or about 37%, at or about 38%, at or about 39%, at or about 40%, at or about 30%, at or about 30%, at or about 40% after storage compared to the viscosity prior to storage. In some embodiments, the storage is at refrigerated temperatures of 2-8° C., such as 5° C. In some embodiments, the storage is at elevated temperatures, such as 30° C. or 40° C. In some embodiments, the storage is at or about 25° C. In some embodiments, the storage is at refrigerated temperatures of 2-8° C., such as 5° C., for at least or about 12 weeks, about 24 weeks, 36 weeks, 48 weeks, 1 year (e.g. 52 weeks), 2 years or 3 years. In some embodiments, the storage is at refrigerated temperatures of 2-8° C., such as 5° C., for at least or about 12 weeks. In some embodiments, the storage is at refrigerated temperatures of 2-8° C., such as 5° C., for at least or about 1 year. In some embodiments, the storage is at refrigerated temperatures of 2-8° C., such as 5° C., for at least or about 12 weeks. In some embodiments, the storage is at elevated temperatures, such as 30° C. or 40° C., for up to 6 weeks or more. In some embodiments, the storage is at room temperature, such as between about 23° C. and 28° C., such as at or about 25° C., for at least or about 1 weeks, about 2 weeks, 3 weeks, 4 weeks, 5 weeks, 6 weeks, 8 weeks, 10 weeks, or 12 weeks, or any value between any of the foregoing. In some embodiments, the storage is at about 25° C., for at least or about 1 week. In some embodiments, the storage is at or about 25° C., for at least or about 12 weeks. In embodiments herein, the viscosity is assessed when the formulation is at or about 25° C. when the formulation was previously stored at a lower or higher temperature.

### B. Stability of Forms of the TACI-Fc Protein in the Formulation

In particular embodiments, the TACI-Fc fusion protein in the pharmaceutical formulations herein is a dimer composed of two identical receptor Fc fusion protein chains (i.e., a homodimer), each with a TACI domain-Fc fusion linked by covalent disulfide bonds. This homodimeric form of the TACI-Fc is also referred to as the “main species” or “main form” herein, as the TACI-Fc typically assembles into this dimer form.

It is understood that the particular molecular weight of the “main” species is dependent on the sequence of the TACI-Fc fusion protein. As provided herein, the TACI polypeptide in the TACI-Fc fusion protein can comprise the ECD of the TACI polypeptide or can comprise a portion thereof comprising one or both of a CRD I domain and a CRD2 domain. The skilled artisan knows that the molecular weight of a TACI-Fc dimer composed of two identical receptor Fc fusion protein chains, each with a variant TACI domain-Fc fusion linked by covalent disulfide bonds will depend on the size of the TACI polypeptides in the dimer. An exemplary main species of a TACI-Fc dimer composed of two identical TACI-Fc fusion protein chains of SEQ ID NO. 167 linked by covalent disulfide bonds is about 62.6 kDa. A main species of a TACI-Fc dimer composed of two TACI-Fc fusion protein chains with a TACI polypeptide that is longer than the CRD2 of TACI will have a higher molecular weight than 62.6 kDa. For example, a main species of a TACI-Fc dimer composed of two TACI-Fc fusion protein chains where the TACI polypeptide consists of the full TACI ECD will have a molecular weight of at or about 116.4 kDa. The skilled artisan readily can calculate the molecular weight of the main species of a TACI-Fc protein herein using the molecular weight of the particular TACI-Fc polypeptide chain and the number of chains in the species (e.g., two TACI-Fc polypeptide chains in the main species).

The physical properties of protein formulations at a high protein concentration can impact the ease with which they can be administered. Formation of high molecular weight (HMW) species of the TACI-Fc fusion protein, for example due to aggregation, can result in decreased efficacy or other adverse effects, such as those as described herein. For example, under certain conditions and with certain formulations, upon storage and/or incubation at refrigerated (e.g., 2-8° C.) or elevated temperatures (e.g., 30° C. or 40° C.), the TACI-Fc fusion protein can form high molecular weight species composed of more than 2 TACI-Fc fusion polypeptide chains, such as TACI-Fc fusion proteins with a molecular weight greater than about 62.6 kDa. In other examples, upon storage and/or incubation at refrigerated (e.g., 2-8° C.) or elevated temperatures (e.g., 30° C. or 40° C.), the TACI-Fc fusion protein can form low molecular weight species (LMW), for example due to degradation, composed of less than 2 TACI-Fc fusion polypeptide chains, such as TACI-Fc fusion proteins with a molecular weight less than 62.6 kDa. Thus, after storage the amount of TACI-Fc protein in the main species can decrease and the amount of TACI-Fc protein that is a HMW and/or LMW species can increase.

In the provided embodiments, the TACI-Fc fusion protein in the formulation is stable. In some embodiments, the TACI-Fc formulations described herein are characterized by low percentage of protein aggregation. In some embodiments, the TACI-Fc formulations described herein are characterized by low percentage of high molecular weight species of the TACI-Fc fusion protein.

In some embodiments the form of the TACI-Fc fusion protein in the formulation is assessed by methods known in the art. Various assays are known and include, but are not limited to, for example, Size Exclusion Chromatography (SEC), reduced capillary electrophoresis sodium dodecyl sulfate (rCE-SDS), Imaged capillary isoelectric focusing (icIEF), dynamic light scattering (DLS), analytical ultracentrifugation, field flow fractionation (FFF), turbidity measurements, NMR spectroscopy, electron microscopy, light obscuration, flow imaging microscopy, and background membrane imaging. For example, in some embodiments, assays known in the art, such as SEC or rCE-SDS, can be used to determine aggregation and degradation of a TACI-Fc fusion protein by assessing the forms of the protein.

For example, size exclusion chromatography (SEC) can be used in some embodiments to determine the percentage of the protein present in the formulation that is in the main species, as the area % under a curve that includes the TACI-Fc fusion protein in a form of a homodimer comprising two identical copies of the TACI-Fc fusion protein linked by a covalent disulfide bond as the main species. The SEC assay can be used to measure the total percentage of high molecular weight (HMW) and low molecular weight (LMW) TACI-Fc species in a pharmaceutical formulation to determine how the formulation components affect aggregation and stability of the TACI-Fc. SEC separates molecules based upon hydrodynamic radius which correlates with the molecular weight of an analyte. In embodiments herein, the forms of the TACI-Fc fusion protein in the formulation are determined by SEC. In particular embodiments, the amount of the TACI-Fc fusion protein in the main form in the formulation is determined by SEC. In particular embodiments, the amount of the TACI-Fc fusion protein as a high molecular weight species in the formulation is determined by SEC. In particular embodiments, the amount of the TACI-Fc fusion protein as a low molecular weight (LMW) species and/or as a high molecular weight (HMW) species in the formulation is determined by SEC. In some embodiments, the amount of the TACI-Fc fusion protein in the main species, HMW species, and/or LMW species, for example, as measured by SEC or rCE-SDS, or other known techniques, is expressed in terms of an area % under a curve that includes the main species and those with a higher and/or lower molecular weight than the main species. Thus, in some embodiments, a “percentage” of the protein in a particular molecular weight species refers to an area percentage (area %).

In some embodiments, the percentage of the TACI-Fc protein in the high molecular weight species in the formulation is less than about 5% of the TACI-Fc protein in the formulation. In some embodiments, the percentage of the TACI-Fc protein in the high molecular weight species is less than about 5%, 4%, 3%, 2%, 1.5%, or 1% of the TACI-Fc protein in the formulation. In some embodiments, the percentage of the TACI-Fc protein in the high molecular weight species is at or about 5%, 4%, 3%, 2%, 1.5%, or 1%, or any value between any of the foregoing, of the TACI-Fc protein in the formulation.

In some embodiments, the percentage of the TACI-Fc protein in the high molecular weight species in the formulation is less than about 5%, 4%, 3%, 2%, 1.5%, 1%, 0.9%, 0.8%, 0.7%, 0.6%, 0.5%, 0.4%, 0.3%, 0.2% or 0.1% of the TACI-Fc protein in the formulation following storage at about 2° C. to about 8° C., such as at or about 5° C. for 2 months. In some embodiments, the percentage of the TACI-Fc protein in the high molecular weight species in the formulation is less than about 5%, 4%, 3%, 2%, 1.5%, 1%, 0.9%, 0.8%, 0.7%, 0.6%, 0.5%, 0.4%, 0.3%, 0.2% or 0.1% of the TACI-Fc protein in the formulation following storage at about 2° C. to about 8° C., such as at or about 5° C., for 6 months. In some embodiments, the percentage of the TACI-Fc protein in the high molecular weight species in the formulation is less than about 5%, 4%, 3%, 2%, 1.5%, 1, 0.9%, 0.8%, 0.7%, 0.6%, 0.5%, 0.4%, 0.3%, 0.2% or 0.1% of the TACI-Fc protein in the formulation following storage at about 2° C. to about 8° C., such as at or about 5° C. for 12 months. In some embodiments, the percentage of the TACI-Fc protein in the high molecular weight species in the formulation is less than about 5%, 4%, 3%, 2%, 1.5%, 1%, 0.9%, 0.8%, 0.7%, 0.6%, 0.5%, 0.4%, 0.3%, 0.2% or 0.1% of the TACI-Fc protein in the formulation following storage about 2° C. to about 8° C., such as at or about 5° C. for 24 months. In some embodiments, the percentage of the TACI-Fc protein in the high molecular weight species in the formulation is less than about 5%, 4%, 3%, 2%, 1.5%, 1%, 0.9%, 0.8%, 0.7%, 0.6%, 0.5%, 0.4%, 0.3%, 0.2% or 0.1% of the TACI-Fc protein in the formulation following storage at about 2° C. to about 8° C., such as at or about 5° C. for 36 months. In some embodiments, the percentage of the TACI-Fc protein in the high molecular weight species in the formulation is less than about 5%, 4%, 3%, 2%, 1.5%, 1%, 0.9%, 0.8%, 0.7%, 0.6%, 0.5%, 0.4%, 0.3%, 0.2% or 0.1% of the TACI-Fc protein in the formulation following storage about 2° C. to about 8° C., such as at or about 5° C. for 48 months. In particular embodiments, the percentage of the TACI-Fc protein in the high molecular weight species in the formulation is less than about 0.5% of the TACI-Fc protein in the formulation following storage at between 2° C. to about 8° C., such as at or about 5° C. for 3 months. In particular embodiments, the percentage of the TACI-Fc protein in the high molecular weight species in the formulation is less than about 2% of the TACI-Fc protein in the formulation following storage at between 2° C. to about 8° C., such as at or about 5° C. for 12 months.

In some embodiments, the percentage of the TACI-Fc protein in the high molecular weight species in the formulation is less than about 5%, 4%, 3%, 2%, 1.5%, 1%, 0.9%, 0.8%, 0.7%, 0.6%, 0.5%, 0.4%, 0.3%, 0.2% or 0.1% of the TACI-Fc protein in the formulation following storage at about 25° C. for 2 months. In some embodiments, the percentage of the TACI-Fc protein in the high molecular weight species in the formulation is less than about 5%, 4%, 3%, 2%, 1.5%, 1%, 0.9%, 0.8%, 0.7%, 0.6%0, 0.5%, 0.4%, 0.3%, 0.2% or 0.1% of the TACI-Fc protein in the formulation following storage at 25° C. for 6 months. In some embodiments, the percentage of the TACI-Fc protein in the high molecular weight species in the formulation is less than about 5%, 4%, 3%, 2%, 1.5%, or 1% of the TACI-Fc protein in the formulation following storage at about 25° C. for 12 weeks. In particular embodiments, the percentage of the TACI-Fc protein in the high molecular weight species in the formulation is less than about 0.5% of the TACI-Fc protein in the formulation following storage at about 25° C. for 12 weeks.

In some embodiments, the formulation described herein comprises no more than about 5% HMW species following storage at about 5° C. for two months as measured by size exclusion chromatography. In some embodiments, the formulation described herein comprises no more than about 5% HMW species following storage at about 25° C. for two months as measured by size exclusion chromatography. In some embodiments, the formulation described herein comprises no more than about 5% HMW species following storage at about 5° C. for six months as measured by size exclusion chromatography. In some embodiments, the formulation described herein comprises no more than about 5% HMW species following storage at about 25° C. for six months as measured by size exclusion chromatography. In some embodiments, the formulation described herein comprises no more than about 5% HMW species following storage at about 5° C. for eighteen months as measured by size exclusion chromatography. In some embodiments, the formulation described herein comprises no more than about 5% HMW species following storage at about 5° C. for twenty four months as measured by size exclusion chromatography. In some embodiments, the formulation described herein comprises no more than about 5% HMW species following storage at about 5° C. for thirty six months as measured by size exclusion chromatography. In some embodiments, the formulation described herein comprises no more than about 5% HMW species following storage at about 5° C. for forty-eight months as measured by size exclusion chromatography. In some embodiments, the analysis is conducted on a liquid formulation upon storage of the formulation under suitable storage conditions.

In some embodiments, the percentage of high molecular weight (HMW) species of the TACI-Fc fusion protein is calculated by the rate of formation of the HMW species over time, such as the rate of formation of the HMW species per week. In some embodiments, the rate of change of HMW species is determined using linear regression by the least squares fit method of the data beginning with time 0. As set forth herein, a percent rate of change that is a positive value indicates an increase of this amount of TACI-Fc species. For example, a rate of change of the TACI-Fc protein in the high molecular weight species per week of at or about 0.01% per week indicates that there was an increase of about 0.01% of the amount of HMW TACI-Fc each week.

In some embodiments, the rate of change of the TACI-Fc protein in the high molecular weight species per week is less than 1%, such as between about 0.01%-0.20%, such as between about 0.01%-0.175%, between about 0.01%-0.15%, between about 0.01%-0.125%, between about 0.01%-0.1%, between about 0.01%-0.075%, between about 0.01%-0.05%, between about 0.01%-0.025%, between about 0.01%-0.02%, between about 0.015%-0.175%, between about 0.015%-0.15%, between about 0.015%-0.125%, between about 0.015%-0.1%, between about 0.015%-0.075%, between about 0.015%-0.05%, between about 0.015%-0.025%, between about 0.015%-0.02%, between about 0.02%-0.175%, between about 0.02%-0.15%, between about 0.02%-0.125%, between about 0.02%-0.1%, between about 0.02%-0.075%, between about 0.02%-0.05%, between about 0.02%-0.025%, between about 0.04%-0.175%, between about 0.04%-0.15%, between about 0.04%-0.16%, between about 0.04%-0.17%, between about 0.04%-0.125%, between about 0.04%-0.1%, between about 0.04%-0.075%, or between about 0.04%-0.05%. In some embodiments, the rate of change of the TACI-Fc protein in the high molecular weight species per week is less than at or about 0.010%, 0.015%, 0.02%, 0.025%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, or any value between any of the foregoing. In some embodiments, the rate of change of the TACI-Fc protein in the high molecular weight species is less than at or about 0.01% per week. In other examples, the rate of change or percentage of the TACI-Fc protein in the high molecular weight species is at or about 0.01% per week.

In particular examples the rate of change of the HMW species is assessed by SEC. In some embodiments, the rate of change of the TACI-Fc protein in the high molecular weight species per week is less than at or about 0.010%, 0.015%, 0.02%, 0.025%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, or any value between any of the foregoing, as assessed by SEC.

In particular examples the rate of change of the HMW species is assessed by rCE-SDS. In some embodiments, the rate of change of the TACI-Fc protein in the high molecular weight species per week is less than at or about 0.010%, 0.015%, 0.02%, 0.025%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, or any value between any of the foregoing, as assessed by rCE-SDS.

In some embodiments, the formulation described herein comprises no more than about 5% HMW species as measured by size exclusion chromatography. In some embodiments, the formulation described herein comprises no more than about 5% HMW species upon freeze/thaw of the liquid formulation (e.g., a freeze/thaw cycle of −20° C./25° C.) as measured by size exclusion chromatography.

In some embodiments, the TACI-Fc formulations described herein are characterized by low percentage of low molecular weight species of the TACI-Fc fusion protein, for example, as measured by SEC or other methods provided above. In some embodiments, the percentage of low molecular weight species is provided as an area % as described above.

In some embodiments, the percentage of the TACI-Fc protein in the low molecular weight species in the formulation is less than 5% of low molecular weight TACI-Fc species. In some embodiments, the percentage of the TACI-Fc protein in the low molecular weight species is less than about 5%, 4%, 3%, 2%, 1.5%, or 1% of the TACI-Fc protein in the formulation. In some embodiments, the percentage of the TACI-Fc protein in the low molecular weight species is at or about 5%, 4%, 3%, 2%, 1.5%, or 1%, or any value between any of the foregoing, of the TACI-Fc protein in the formulation. In some embodiments the percentage of TACI-Fc protein in the low molecular weight species in the formulation is measured by size exclusion chromatography. In some embodiments the percentage of TACI-Fc protein in the low molecular weight species in the formulation is measured by rCE.

In some embodiments, the percentage of the TACI-Fc protein in the low molecular weight species in the formulation is less than about 5%, 4%, 3%, 2%, 1.5%, or 1% of the TACI-Fc protein in the formulation following storage at about 5° C. for 2 months. In some embodiments, the percentage of the TACI-Fc protein in the low molecular weight species in the formulation is less than about 5%, 4%, 3%, 2%, 1.5%, or 1% of the TACI-Fc protein in the formulation following storage at about 5° C. for 6 months. In some embodiments, the percentage of the TACI-Fc protein in the low molecular weight species in the formulation is less than about 5%, 4%, 3%, 2%, 1.5%, or 1% of the TACI-Fc protein in the formulation following storage at about 5° C. for 12 months. In some embodiments, the percentage of the TACI-Fc protein in the low molecular weight species in the formulation is less than about 5%, 4%, 3%, 2%, 1.5%, or 1% of the TACI-Fc protein in the formulation following storage at about 5° C. for 24 months. In some embodiments, the percentage of the TACI-Fc protein in the low molecular weight species in the formulation is less than about 5%, 4%, 3%, 2%, 1.5%, or 1% of the TACI-Fc protein in the formulation following storage at about 5° C. for 36 months. In some embodiments, the percentage of the TACI-Fc protein in the low molecular weight species in the formulation is less than about 5%, 4%, 3%, 2%, 1.5%, or 1% of the TACI-Fc protein in the formulation following storage at about 5° C. for 48 months. In particular embodiments, the percentage of the TACI-Fc protein in the low molecular weight species in the formulation is less than about 0.5% of the TACI-Fc protein in the formulation following storage at between 2° C. to about 8° C., such as at or about 5° C. for 3 months. In particular embodiments, the percentage of the TACI-Fc protein in the low molecular weight species in the formulation is less than about 0.5% of the TACI-Fc protein in the formulation following storage at between 2° C. to about 8° C., such as at or about 5° C. for 12 months. In some embodiments the percentage of TACI-Fc protein in the low molecular weight species in the formulation is measured by size exclusion chromatography. In some embodiments the percentage of TACI-Fc protein in the low molecular weight species in the formulation is measured by rCE.

In some embodiments, the percentage of the TACI-Fc protein in the low molecular weight species in the formulation is less than about 10%, 9%, 8%, 7%, 6%, 5%, 4%, 3%, 2%, 1.5%, or 1% of the TACI-Fc protein in the formulation following storage at about 25° C. for 2 months. In some embodiments, the percentage of the TACI-Fc protein in the low molecular weight species in the formulation is less than about 5%, 4%, 3%, 2%, 1.5%, or 1% of the TACI-Fc protein in the formulation following storage at about 25° C. for 6 months. In some embodiments the percentage of TACI-Fc protein in the low molecular weight species in the formulation is measured by size exclusion chromatography. In some embodiments the percentage of TACI-Fc protein in the low molecular weight species in the formulation is measured by rCE.

In some embodiments, the formulation described herein comprises no more than about 5% LMW species following storage at about 5° C. for two months as measured by size exclusion chromatography. In some embodiments, the formulation described herein comprises no more than about 5% LMW species following storage at about 25° C. for two months as measured by size exclusion chromatography. In some embodiments, the formulation described herein comprises no more than about 5% LMW species following storage at about 5° C. for six months as measured by size exclusion chromatography. In some embodiments, the formulation described herein comprises no more than about 5% LMW species following storage at about 25° C. for six months as measured by size exclusion chromatography. In some embodiments, the formulation described herein comprises no more than about 5% LMW species following storage at about 5° C. for eighteen months as measured by size exclusion chromatography. In some embodiments, the formulation described herein comprises no more than about 5% LMW species following storage at about 5° C. for twenty four months as measured by size exclusion chromatography. In some embodiments, the formulation described herein comprises no more than about 5% LMW species following storage at about 5° C. for thirty six months as measured by size exclusion chromatography. In some embodiments, the formulation described herein comprises no more than about 5% LMW species following storage at about 5° C. for forty eight months as measured by size exclusion chromatography. In some embodiments, the analysis is conducted on a liquid formulation upon storage of the formulation under suitable storage conditions.

In some embodiments, the percentage of low molecular weight (LMW) species of the TACI-Fc fusion protein is calculated by the rate of formation of the LMW species over time, such as the rate of formation of the LMW species per week. In some embodiments the rate of change of a LMW species is calculated by linear regression using least squares fit of the data. In such embodiments, error bars are the calculated standard error in the rate calculation. In some embodiments, the rate of change of a LMW species is calculated by taking the average percent of a particular species over a set time period and subtracting the percent of the species at time 0. As set forth herein, a percent rate of change that is a positive value indicates an increase of this amount of TACI-Fc species. For example, a rate of change of the TACI-Fc protein in the low molecular weight species per week of at or about 0.01% per week indicates that there was an increase of about 0.01% of the amount of LMW TACI-Fc each week.

In some embodiments, the rate of change of the TACI-Fc protein in the low molecular weight species per week is less than 1%, such as between about 0.01%-0.20%, such as between about 0.01%-0.175%, between about 0.01%-0.15%, between about 0.01%-0.125%, between about 0.01%-0.1%, between about 0.01%-0.075%, between about 0.01%-0.05%, between about 0.01%-0.025%, between about 0.01%-0.02%, between about 0.015%-0.175%, between about 0.015%-0.15%, between about 0.015%-0.125%, between about 0.015%-0.1%, between about 0.015%-0.075%, between about 0.015%-0.05%, between about 0.015%-0.025%, between about 0.015%-0.02%, between about 0.02%-0.175%, between about 0.02%-0.15%, between about 0.02%-0.125%, between about 0.02%-0.1%, between about 0.02%-0.075%, between about 0.02%-0.05%, between about 0.02%-0.025%, between about 0.04%-0.175%, between about 0.04%-0.15%, between about 0.04%-0.16%, between about 0.04%-0.17%, between about 0.04%-0.125%, between about 0.04%-0.1%, between about 0.04%-0.075%, or between about 0.04%-0.05%. In some embodiments, the rate of change of the TACI-Fc protein in the low molecular weight species per week is less than at or about 0.010%, 0.015%, 0.02%, 0.025%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, or any value between any of the foregoing. In some embodiments, the rate of change of the TACI-Fc protein in the low molecular weight species is less than at or about 0.01% per week. In other examples, the rate of change or percentage of the TACI-Fc protein in the low molecular weight species is at or about 0.01% per week. In any of the preceding embodiments, the rate of change of the TACI-Fc protein in the low molecular weight species per week is assessed by size exclusion chromatography.

In particular examples the rate of change of the LMW species is assessed by SEC. In some embodiments, the rate of change of the TACI-Fc protein in the low molecular weight species per week is less than at or about 0.010%, 0.015%, 0.02%, 0.025%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, or any value between any of the foregoing, as assessed by SEC.

In particular examples the rate of change of the LMW species is assessed by rCE-SDS. In some embodiments, the rate of change of the TACI-Fc protein in the low molecular weight species is less than at or about 0.010%, 0.015%, 0.02%, 0.025%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, or any value between any of the foregoing, as assessed by rCE-SDS.

In some embodiments, the formulation described herein comprises no more than about 5% LMW species as measured by size exclusion chromatography. In some embodiments, the formulation described herein comprises no more than about 5% LMW species upon freeze/thaw of the liquid formulation (e.g., a freeze/thaw cycle of −20° C./25° C.) as measured by size exclusion chromatography.

In some embodiments, the TACI-Fc formulations described herein are characterized by low percentage of total HMW and LMW species of the TACI-Fc fusion protein combined. In such examples, if 5% of the total TACI-Fc protein in the formulation is in the HMW and LMW species, then 95% of the TACI-Fc protein is in the main species form.

In some embodiments, the percentage of the TACI-Fc protein in the low molecular weight species and high molecular weight species in the formulation is less than 5% of the total TACI-Fc protein in the formulation. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation. In some embodiments, the percentage of the TACI-Fc protein in the low molecular weight species and high molecular weight species is at or about 5%, 4%, 3%, 2%, 1.5%, or 1%, or any value between any of the foregoing, of the TACI-Fc protein in the formulation.

In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 5° C. for 2 months. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 5° C. for 6 months. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 5° C. for 12 months. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 5° C. for 24 months. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 5° C. for 36 months. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 5° C. for 48 months.

In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 25° C. for 2 months. In some embodiments, the percentage of the TACI-Fc protein in the low molecular weight species and high molecular weight species in the formulation is less than about 5%, 4%, 3%, 2%, 1.5%, or 1% of the TACI-Fc protein in the formulation following storage at about 25° C. for 6 months.

In some embodiments, the formulation described herein comprises no more than about 5% LMW species and HMW species combined following storage at about 5° C. for two months as measured by size exclusion chromatography. In some embodiments, the formulation described herein comprises no more than about 5% LMW species and HMW species combined following storage at about 25° C. for two months as measured by size exclusion chromatography. In some embodiments, the formulation described herein comprises no more than about 5% LMW species and HMW species combined following storage about 2° C. to about 8° C., such as at or about 5° C. for six months as measured by size exclusion chromatography. In some embodiments, the formulation described herein comprises no more than about 5% LMW species and HMW species combined following storage at about 25° C. for six months as measured by size exclusion chromatography. In some embodiments, the formulation described herein comprises no more than about 5% LMW species and HMW species combined following storage about 2° C. to about 8° C., such as at or about 5° C. for eighteen months as measured by size exclusion chromatography. In some embodiments, the formulation described herein comprises no more than about 5% LMW species and HMW species combined following storage about 2° C. to about 8° C., such as at or about 5° C. for twenty four months as measured by size exclusion chromatography. In some embodiments, the formulation described herein comprises no more than about 5% LMW species and HMW species combined following storage at about 5° C. for thirty six months as measured by size exclusion chromatography. In some embodiments, the formulation described herein comprises no more than about 5% LMW species and HMW species combined following storage about 2° C. to about 8° C., such as at or about 5° C. for forty eight months as measured by size exclusion chromatography. In some embodiments, the analysis is conducted on a liquid formulation upon storage of the formulation under suitable storage conditions.

As described herein, the TACI-Fc fusion protein in the pharmaceutical formulations typically assembles as a dimer consisting of two identical TACI-Fc fusion protein chains, each with a TACI domain-Fc fusion linked by covalent disulfide bonds. In the formulations provided herein, the main form of the TACI-Fc protein comprises the majority of the TACI-Fc protein in the formulation.

In some embodiments, the percentage of the main species of TACI-Fc protein in the formulation is at least or is about 90% of the total TACI-Fc protein in the formulation, such as less than or about 10% of the TACI-Fc protein is in the HMW and LMW forms. In some embodiments, the percentage of the main species of TACI-Fc protein in the formulation is at least or is about 90% of the total TACI-Fc protein in the formulation, such as less than or about 10% of the TACI-Fc protein is in the HMW and LMW forms, as measured by rCE-SDS. In some embodiments, the percentage of the main species of TACI-Fc protein in the formulation is at least or is about 95% of the total TACI-Fc protein in the formulation, such as less than or about 5% of the TACI-Fc protein is in the HMW and LMW forms. In some embodiments, the percentage of the main species of TACI-Fc protein is between at or about 90%-99%, 90%-95%, or 95%-99% of the TACI-Fc protein in the formulation. In some embodiments, the percentage of the main species of TACI-Fc protein is at least or at least about 90%, 91%, 92%, 93%, 94%, 95%, 96%, 97%, 98%, or 99%, or any value between any of the foregoing, of the TACI-Fc protein in the formulation. In some embodiments, the percentage of the main species of TACI-Fc protein is at or about 5%, 4%, 3%, 2%, 1.5%, or 1%, or any value between any of the foregoing, of the TACI-Fc protein in the formulation.

In some embodiments, the percentage of the main species of TACI-Fc protein in the formulation is at least about 90%, 91%, 92%, 93%, 94%, 95%, 96%, 97%, 98%, or 99% of the TACI-Fc protein in the formulation following storage at about 5° C. for 2 months. In some embodiments, the percentage of the main species of TACI-Fc protein in the formulation is at least about 90%, 91%, 92%, 93%, 94%, 95%, 96%, 97%, 98%, or 99% of the TACI-Fc protein in the formulation following storage at about 5° C. for 6 months. In some embodiments, the percentage of the main species of TACI-Fc protein in the formulation is at least about 90%, 91%, 92%, 93%, 94%, 95%, 96%, 97%, 98%, or 99% of the TACI-Fc protein in the formulation following storage at about 5° C. for 12 months. In some embodiments, the percentage of the main species of TACI-Fc protein in the formulation is at least about 90%, 91%, 92%, 93%, 94%, 95%, 96%, 97%, 98%, or 99% of the TACI-Fc protein in the formulation following storage at about 5° C. for 24 months. In some embodiments, the percentage of the main species of TACI-Fc protein in the formulation is at least about 90%, 91%, 92%, 93%, 94%, 95%, 96%, 97%, 98%, or 99% of the TACI-Fc protein in the formulation following storage at about 5° C. for 36 months. In some embodiments, the percentage of the main species of TACI-Fc protein in the formulation is at least about 90%, 91%, 92%, 93%, 94%, 95%, 96%, 97%, 98%, or 99% of the TACI-Fc protein in the formulation following storage at about 5° C. for 48 months. In particular embodiments, the percentage of the main species of TACI-Fc protein in the formulation is at least about 99% of the TACI-Fc protein in the formulation following storage at about 2° C. to about 8° C., such as at or about 5° C., for 3 months. In particular embodiments, the percentage of the main species of TACI-Fc protein in the formulation is at least about 98% of the TACI-Fc protein in the formulation following storage at about 2° C. to about 8° C., such as at or about 5° C., for 12 months. In particular embodiments, the percentage of the main species of TACI-Fc protein in the formulation is at least about 98% of the TACI-Fc protein in the formulation following storage at about 5° C., for 3 months. In some embodiments the percentage of TACI-Fc protein in the main species in the formulation is measured by size exclusion chromatography. In some embodiments the percentage of TACI-Fc protein in the main species in the formulation is measured by rCE or SEC. In some embodiments, the percentage is an area % as described above.

In some embodiments, the percentage of the main species of TACI-Fc protein in the formulation at least about 90%, 91%, 92%, 93%, 94%, 95%, 96%, 97%, 98%, or 99% of the TACI-Fc protein in the formulation following storage at about 25° C. for 2 months. In some embodiments, the percentage of the main species of TACI-Fc protein in the at least about 90%, 91%, 92%, 93%, 94%, 95%, 96%, 97%, 98%, or 99% of the TACI-Fc protein in the formulation following storage at about 25° C. for 6 months. In some embodiments the percentage of the main species of TACI-Fc protein in the formulation is measured by size exclusion chromatography. In some embodiments the percentage of the main species of TACI-Fc protein in the formulation is measured by rCE or SEC. In some embodiments, the percentage is an area % as described above.

In some embodiments, the formulation described herein comprises at least about 95% main TACI-Fc protein species following storage at about 5° C. for two months as measured by size exclusion chromatography. In some embodiments, the formulation described herein comprises at least about 95% main TACI-Fc protein species following storage at about 25° C. for two months as measured by size exclusion chromatography. In some embodiments, the formulation described herein comprises at least about 95% main TACI-Fc protein species following storage at about 5° C. for six months as measured by size exclusion chromatography. In some embodiments, the formulation described herein comprises at least about 95% main TACI-Fc protein species following storage at about 25° C. for six months as measured by size exclusion chromatography. In some embodiments, the formulation described herein comprises at least about 95% main TACI-Fc protein species following storage at about 5° C. for eighteen months as measured by size exclusion chromatography. In some embodiments, the formulation described herein comprises at least about 95% main TACI-Fc protein species following storage at about 5° C. for twenty four months as measured by size exclusion chromatography. In some embodiments, the formulation described herein comprises at least about 95% main TACI-Fc protein species following storage at about 5° C. for thirty six months as measured by size exclusion chromatography. In some embodiments, the formulation described herein comprises at least about 95% main TACI-Fc protein species following storage at about 5° C. for forty eight months as measured by size exclusion chromatography. In some embodiments, the analysis is conducted on a liquid formulation upon storage of the formulation under suitable storage conditions.

In some embodiments, the loss of the main TACI-Fc protein species is calculated by the rate of loss of the main TACI-Fc protein species over time, such as the loss of the main TACI-Fc protein species per week. In some embodiments, the rate of change of a main TACI-Fc protein species is calculated by taking the average percent of a main species over a set time period and subtracting the percent of the species at time 0. As set forth herein, a percent rate of change that is a negative value indicates a decrease of this amount of TACI-Fc protein species over time. For example, a rate of change of the TACI-Fc in the main species per week of at or about −0.01% per week indicates that there was a decrease of about 0.01% of the amount of main TACI-Fc protein species each week.

In some embodiments, the rate of change of the TACI-Fc protein in the main species per week is less than −1%, such as between about −0.01% and −0.20%, such as between about −0.01% and −0.175%, between about −0.01% and −0.15%, between about −0.01% and −0.125%, between about −0.01% and −0.1%, between about −0.01% and −0.075%, between about −0.01% and −0.05%, between about −0.01% and −0.025%, between about −0.01% and −0.02%, between about −0.015% and −0.175%, between about −0.015% and −0.15%, between about −0.015% and −0.125%, between about −0.015% and −0.1%, between about −0.015% and −0.075%, between about −0.015% and −0.05%, between about −0.015% and −0.025%, between about −0.015% and −0.02%, between about −0.02% and −0.175%, between about −0.02% and −0.15%, between about −0.02% and −0.125%, between about −0.02% and −0.1%, between about −0.02% and −0.075%, between about −0.02% and −0.05%, between about −0.02% and −0.025%, between about −0.04% and −0.175%, between about −0.04% and −0.15%, between about −0.04% and −0.16%, between about −0.04% and −0.17%, between about −0.04% and −0.125%, between about −0.04% and −0.1%, between about −0.04% and −0.075%, or between about −0.04% and −0.05%. In some embodiments, the rate of change of the TACI-Fc protein in the main species per week is less than at or about −0.010%, −0.015%, −0.02%, −0.025%, −0.03%, −0.04%, −0.05%, −0.06%, −0.07%, −0.08%, −0.09%, −0.10%, −0.11%, −0.12%, −0.13%, −0.14%, −0.15%, −0.16%, −0.17%, −0.18%, −0.19%, or −0.20%, or any value between any of the foregoing. In some embodiments, the rate of change of the TACI-Fc protein in the main species is less than at or about −0.01% per week. In other examples, the rate of change or percentage of the TACI-Fc in the main species is at or about −0.01% per week.

In particular examples the rate of change of the TACI-Fc protein in the main species is assessed by SEC. In some embodiments, the rate of change of the TACI-Fc protein in the main species per week is less than at or about −0.010%, −0.015%, −0.02%, −0.025%, −0.03%, −0.04%, −0.05%, −0.06%, −0.07%, −0.08%, −0.09%, −0.10%, −0.11%, −0.12%, −0.13%, −0.14%, −0.15%, −0.16%, −0.17%, −0.18%, −0.19%, −0.20%, or any value between any of the foregoing, as assessed by SEC.

In particular examples the rate of change of the TACI-Fc protein in the main species is assessed by rCE-SDS. In some embodiments, the rate of change of the TACI-Fc in the main species per week is less than at or about −0.010%, −0.015%, −0.02%, −0.025%, −0.03%, −0.04%, −0.05%, −0.06%, −0.07%, −0.08%, −0.09%, −0.10%, −0.11%, −0.12%, −0.13%, −0.14%, −0.15%, −0.16%, −0.17%, −0.18%, −0.19%, −0.20%, or any value between any of the foregoing, as assessed by rCE-SDS.

In some embodiments, the formulation described herein comprises at least about 95% main TACI-Fc protein species as measured by size exclusion chromatography. In some embodiments, the formulation described herein comprises at least about 95% main TACI-Fc protein species upon freeze/thaw of the liquid formulation (e.g., a freeze/thaw cycle of −20° C./25° C.) as measured by size exclusion chromatography.

In some embodiments, at least about 98% of the TACI-Fc protein in the formulation described herein is in the main dimer form as measured by size exclusion chromatography.

In some embodiments, at least about 98% of the TACI-Fc protein in the formulation described herein is in the main dimer form as measured by rCE.

In some embodiments, at least about 98% of the TACI-Fc protein in the formulation described herein is in homodimer (main) form upon freeze/thaw of the liquid formulation (e.g., a freeze/thaw cycle of −20° C./25° C.) as measured by size exclusion chromatography.

In some embodiments, the formulation has the following properties after storage for 12 months in liquid form at a storage temperature of at or about 2° C. to about 8° C., optionally at or about 5° C., and as measured by size exclusion chromatography (SEC) and as expressed in terms of area % under a curve that includes the TACI-Fc fusion protein in a form of a homodimer comprising two identical copies of the TACI-Fc fusion protein linked by a covalent disulfide bond as a main species: (a) the main species is at least 95%, at least 98%, or at least 99% of all observed species under the curve; (b) less than 5%, less than 2%, or less than 1% of species under the curve have a size greater than a size of the main species; (c) less than 5%, less than 2%, or less than 1% of species under the curve have a size less than a size of the main species; (d) the loss of the main species is less than 1%; (e) the increase in species under the curve having a size greater than a size of the main species is less than 1%; and/or (f) the increase in species under the curve having a size less than a size of the main species is less than 1%.

### C. Subvisible Particles in the TACI-Fc Formulation

Subvisible particles can form in protein formulations which can be indicative of particulate impurities and protein aggregation. In some embodiments, the stability is assessed upon storage of the TACI-Fc formulation after storage by assessing the number and size of subvisible particles in the formulation.

In embodiments herein, the TACI-Fc formulations contain a number of subvisible particles that adheres to USP guidelines. In embodiments herein, the TACI-Fc formulations contain less than about 6,000 particles per container ≥10 μm, such as less than 500, 1000, 1500, 2000, 2500, 3000, 3500, 4000, 4500, 5000, or 5,500 particles per container ≥10 μm. In some embodiments the formulation contains less than 500, 1000, 1500, 2000, 2500, 3000, 3500, 4000, 4500, 5000, or 5,500 particles per container ≥10 μm after storage in liquid form at a storage temperature of at or about 2° C. to about 8° C., optionally at or about 5° C., such as 5° C., 20° C., 25° C., 30° C., or 40° C., or any value between any of the foregoing. In some embodiments the formulation contains less than 500, 1000, 1500, 2000, 2500, 3000, 3500, 4000, 4500, 5000, or 5,500 particles per container ≥10 μm after storage for up to about 12 weeks, 24 weeks, 52 weeks, 78 weeks, 104 weeks or more, in liquid form at a storage temperature of at or about 2° C. to about 8° C., optionally at or about 5° C. In some embodiments the formulation contains less than 500, 1000, 1500, 2000, 2500, 3000, 3500, 4000, 4500, 5000, or 5,500 particles per container ≥10 μm after storage in liquid form for up to about 12 weeks, 18 weeks, or 26 weeks, or any value between any of the foregoing, at a storage temperature of at or about 25° C. In some embodiments, the TACI-Fc formulations contain less than about 600 particles/mL having a particle size of >25 μm, such as less than 100, 200, 300, 400, 500, 600, or 1000 particles/mL having a particle size diameter of ≥25 μm, after storage for 12 months in liquid form at a storage temperature of at or about 2° C. to about 8° C., optionally at or about 5° C.

The number of subvisible particles can be assessed using various techniques known in the art. Various assays for assessing subvisible particles include light obscuration based particle counters, flow imaging microscopy particle counters, or background membrane imaging. In some embodiments, a HIAC Liquid Particle Counter (Beckman Coulter) particle analyzer can be used to measure subvisible particles.

### D. Visual Appearance of the Formulations

The visual attributes of pharmaceutical formulations can be assessed to determine if the formulation has undergone detrimental changes upon storage and/or incubation at elevated or refrigerated temperatures.

In embodiments herein, the TACI-Fc formulations are of a color that adheres to European Pharmacopeia Chapter EP 2.2.2. In embodiments herein, the TACI-Fc formulations show a color of <BY4, <BY5, <BY6 or have less color (are more colorless). In embodiments herein, the TACI-Fc formulations show a color of <BY5 after storage in liquid form at a storage temperature of at or about 2° C. to about 8° C., such as at or about 5° C. for up to about 12 weeks, 24 weeks, 52 weeks, 78 weeks, 104 weeks or more. In embodiments herein, the TACI-Fc formulations show a color of <BY4 or <BY5 after storage in liquid form at a storage temperature of at or about 22° C. to about 28° C., such as at or about 25° C. for up to about 12 weeks, or about 24 weeks. In embodiments herein, the TACI-Fc formulations show a color of <BY4 or <BY5 after storage in liquid form at a storage temperature of at or about 40° C. for up to about 12 weeks. In embodiments herein, the TACI-Fc formulations do not show the presence of visible particles. In some embodiments the formulation contains less than 1, 2, 3, 4, 5, 6, 7, 8, 9, or 10 visible particles per container after storage in liquid form for up to about 12 weeks, 24 weeks, 52 weeks, 78 weeks, 104 weeks or more, at a storage temperature of at or about 2° C. to about 8° C., such as at or about 5° C., or at or about 20° C., or at or about 25° C., or at or about 30° C., or at or about 40° C., or any value between any of the foregoing. In some embodiments the formulation contains less than 1, 2, 3, 4, or 5 visible particles per container after storage in liquid form for up to about 12 weeks, 24 weeks, 52 weeks, 78 weeks, 104 weeks or more, at a storage temperature of at or about 2° C. to about 8° C., such as at or about 5° C., or at or about 20° C., or at or about 25° C., or at or about 30° C., or at or about 40° C., or any value between any of the foregoing. Visible particles can be assessed by methods known in the art, such as visual inspection.

### IV. METHODS FOR ASSESSING ACTIVITY AND IMMUNE MODULATION OF IMMUNOMODULATORY PROTEINS

In some embodiments, the TACI-Fc fusion proteins provided herein exhibit immunomodulatory activity. In some embodiments, the provided TACI-Fc fusion protein can modulate B cell activity, such as one or more of B cell proliferation, differentiation or survival.

The function of the TACI-Fc fusion proteins can be examined using a variety of approaches to assess the ability of the proteins to bind to cognate binding partners. For example, TACI fusion proteins may be assessed for binding to APRIL or BAFF. A variety of assays are known for assessing binding affinity and/or determining whether a binding molecule (e.g., immunomodulatory protein) specifically binds to a particular binding partner. It is within the level of a skilled artisan to determine the binding affinity of a binding molecule, e.g., immunomodulatory protein, for a binding partner, e.g., APRIL or BAFF, such as by using any of a number of binding assays that are well known in the art. Various binding assays are known and include, but are not limited to, for example, ELISA K_{D}, KinExA, flow cytometry, and/or surface plasmon resonance devices), including those described herein. Such methods include, but are not limited to, methods involving BIAcore®, Octet®, or flow cytometry. For example, in some embodiments, a BIAcore® instrument can be used to determine the binding kinetics and constants of a complex between two proteins using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

Provided TACI-Fc fusion proteins also can be assessed in any of a variety of assays to assess modulation of B cell activity. One such assay is a cell proliferation assay. Cells are cultured in the presence or absence of a test compound (e.g. immunomodulatory protein), and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). An alternative assay format uses cells that are further engineered to express a reporter gene. The reporter gene is linked to a promoter element that is responsive to the receptor-linked pathway, and the assay detects activation of transcription of the reporter gene. Numerous reporter genes that are easily assayed for in cell extracts are known in the art, for example, the E. coli lacZ, chloroamphenicol acetyl transferase (CAT) and serum response element (SRE) (see, e.g., Shaw et al., Cell 56:563-72, 1989). An exemplary reporter gene is a luciferase gene (de Wet et al., Mol. Cell. Biol. 7:725, 1987). Expression of the luciferase gene is detected by luminescence using methods known in the art (e.g., Baumgartner et al., J. Biol. Chem. 269:29094-101, 1994; Schenborn and Goiffin, Promega Notes 41:11, 1993). Luciferase activity assay kits are commercially available from, for example, Promega Corp., Madison, Wis.

Provided TACI-Fc fusion proteins can be characterized by the ability to inhibit the stimulation of human B cells by soluble APRIL or BAFF, as described by Gross et al, international publication No. WO00/40716. Briefly, human B cells are isolated from peripheral blood mononuclear cells, such as using CD19 magnetic beads separation (e.g. Miltenyi Biotec Auburn, CA). The purified B cells can be incubated under conditions of stimulation, e.g. in the presence of soluble APRIL, and further in the presence of titrated concentration of immunomodulatory protein. The B cells can be labeled with a proliferation dye or can be labeled with 1 μCi ³H-thymidine to measure proliferation. The number of B cells can be determined over time.

Reporter cell lines that express a reporter gene under the operable control of a transcription factor, such as NF-κB, NFAT-1 and AP-1, can be made that express TACI or BCMA. For example, the reporter cell can include Jurkat and other B Lymphoma cell lines. Incubation of these cells with soluble BAFF or APRIL ligands signal through the reporter genes in these constructs. The effect of provided immunomodulatory proteins to modulate this signaling can be assessed.

Well established animal models are available to test in vivo efficacy of provided immunomodulatory proteins in certain disease states, including those involving autoimmune or inflammatory conditions. For example, animal models of autoimmune disease include, for example, MRL-lpr/lpr or NZB×NZW F1 congenic mouse strains which serve as a model of SLE (systemic lupus erythematosus). Such animal models are known in the art, see for example Autoimmune Disease Models A Guidebook, Cohen and Miller eds. Academic Press. Offspring of a cross between New Zealand Black (NZB) and New Zealand White (NZW) mice develop a spontaneous form of SLE that closely resembles SLE in humans. The offspring mice, known as NZBW begin to develop IgM autoantibodies against T-cells at 1 month of age, and by 5-7 months of age, Ig anti-DNA autoantibodies are the dominant immunoglobulin. Polyclonal B-cell hyperactivity leads to overproduction of autoantibodies. The deposition of these autoantibodies, particularly ones directed against single stranded DNA is associated with the development of glomerulonephritis, which manifests clinically as proteinuria, azotemia, and death from renal failure. In some embodiments, proteinuria is determined by the urine total protein to creatinine ratio (UPCR; g/g). Kidney failure is the leading cause of death in mice affected with spontaneous SLE, and in the NZBW strain, this process is chronic and obliterative. The disease is more rapid and severe in females than males, with mean survival of only 245 days as compared to 406 days for the males. While many of the female mice will be symptomatic (proteinuria) by 7-9 months of age, some can be much younger or older when they develop symptoms. The fatal immune nephritis seen in the NZBW mice is very similar to the glomerulonephritis seen in human SLE, making this spontaneous murine model very attractive for testing of potential SLE therapeutics (Putterman and Naparstek, Murine Models of Spontaneous Systemic Lupus Erythematosus, Autoimmune Disease Models: A Guidebook, chapter 14, pp. 217-34, 1994; Mohan et al., J. Immunol. 154:1470-80, 1995; and Daikh et al., J Immunol. 159:3104-08, 1997). Administration of provided immunomodulatory proteins to these mice to evaluate the efficacy to ameliorate symptoms and alterations to the course of disease can be assessed.

Another mouse model of inflammation and lupus-like disease is the bm12 inducible mouse model of SLE (Klarquist and Janssen, 2015. J. Vis. Exp. (105), e53319). Splenocyte suspensions from female I-A^{bm12}B6(C)-H2-Ab1^{bm12}/KhEgJ (‘bm12’) mice are adoptively transferred into female C57BL/6NJ recipient mice. H2-Ab1^{bm12} differs from H2-Ab1^{b} by 3 nucleotides, resulting in alteration of 3 amino acids in the β-chain of the MHC class II I-A molecule. Alloactivation of donor bm12 CD4+ T cells by recipient antigen presenting cells leads to chronic GVHD with symptoms closely resembling SLE, including autoantibody production, changes in immune cell subsets, and mild kidney disease. Glomerulonephritis with immune complex deposition develops late in the model, largely comprised of autoantigens bound to IgG1, IgG2b, IgG2c, and IgG3 antibodies. Endpoints of this model may include concentrations of anti-dsDNA antibodies, select IgG isotypes, blood urea nitrogen (BUN), and creatinine in serum, immune cell subset composition in the spleen and cervical LN, and kidney histology.

In some embodiments, mouse models for Sjögren's syndrome (SjS) can be used. The SjS disease, as well as an accelerated onset of diabetes, can be induced in female diabetes-prone non-obese diabetic (NOD) mice using repeat dosing with anti-mouse (m) PD-L1 antibody, based on a modified version of a protocol published by Zhou et al., 2016 Sci. Rep. 6, 39105. Starting at 6 weeks of age, mice are injected intraperitoneally (IP) on Study Days 0, 2, 4, and 6 with 100 μg of anti-PD-L1 antibody and are treated on various days with provided immunomodulatory proteins. Naïve mice are included as controls for the endpoint analyses. All mice are typically terminated on Study Day 10 and submandibular glands (SMG) and the pancreas from each mouse are collected for histopathology evaluation to assess for signs and severity of sialadenitis and insulitis. Blood glucose levels can be measured on various days.

In some embodiments, mouse models for experimental allergic encephalomyelitis (EAE) can be used. The models resemble human multiple sclerosis and produces demyelination as a result of T-cell activation to neuroproteins such as myelin basic protein (MBP), or proteolipid protein (PLP). Inoculation with antigen leads to induction of CD4+, class II MHC-restricted T-cells (Th1). Changes in the protocol for EAE can produce acute, chronic-relapsing, or passive-transfer variants of the model (Weinberg et al., J. Immunol. 162:1818-26, 1999; Mijaba et al., Cell. Immunol. 186:94-102, 1999; and Glabinski, Meth. Enzym. 288:182-90, 1997). Administration of provided immunomodulatory proteins to ameliorate symptoms and alterations to the course of disease can be assessed.

In some embodiments, a collagen-induced arthritis (CIA) model can be used in which mice develop chronic inflammatory arthritis which closely resembles human rheumatoid arthritis (RA). Since CIA shares similar immunological and pathological features with RA, this makes it an ideal model for screening potential human anti-inflammatory compounds. Another advantage in using the CIA model is that the mechanisms of pathogenesis are known. The T and B cell epitopes on type II collagen have been identified, and various immunological (delayed-type hypersensitivity and anti-collagen antibody) and inflammatory (cytokines, chemokines, and matrix-degrading enzymes) parameters relating to immune-mediating arthritis have been determined and can be used to assess test compound efficacy in the models (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995). Administration of provided immunomodulatory proteins to ameliorate symptoms and alterations to the course of disease can be assessed.

In some embodiments, models for bronchial infection, such as asthma, can be created when mice are injected with ovalbumin and restimulated nasally with antigen which produces an asthmatic response in the bronchi similar to asthma. Administration of provided immunomodulatory proteins to ameliorate symptoms and alterations to the course of disease can be assessed.

In some embodiments, myasthenia gravis (MG) is another autoimmune disease for which murine models are available. MG is a disorder of neuromuscular transmission involving the production of autoantibodies directed against the nicotinic acetylcholine receptor (AChR). MG is acquired or inherited with clinical features including abnormal weakness and fatigue on exertion. A mouse model of MG has been established. (Christadoss et al., Establishment of a Mouse Model of Myasthenia Gravis Which Mimics Human Myasthenia Gravis Pathogenesis for Immune Intervention, in Immunobiology of Proteins and Peptides VIII, Atassi and Bixler, eds., 1995, pp. 195-99.) Experimental autoimmune myasthenia gravis (EAMG) is an antibody mediated disease characterized by the presence of antibodies to AChR. These antibodies destroy the receptor leading to defective neuromuscular electrical impulses, resulting in muscle weakness. In the EAMG model, mice are immunized with the nicotinic acetylcholine receptor. Clinical signs of MG become evident weeks after the second immunization. EAMG is evaluated by several methods including measuring serum levels of AChR antibodies by radioimmunoassay (Christadoss and Dauphinee, J. Immunol. 136:2437-40, 1986; and Lindstrom et al., Methods Enzymol. 74:432-60, 1981), measuring muscle AChR, or electromyography (Wu et al. Protocols in Immunology. Vol. 3, Eds. Coligen, Kruisbeak, Margulies, Shevach, and Strober. John Wiley and Sons, New York, p. 15.8.1, 1997).

Another use for in vivo models includes delivery of an antigen challenge to the animal followed by administration of immunomodulatory proteins and measuring the T and B cell response. T cell dependent and T cell independent immune response can be measured as described in Perez-Melgosa et al., J. Immunol. 163:1123-7, 1999. Immune response in animals subjected to a regular antigen challenge (for example, keyhole limpet hemacyanin (KLH), sheep red blood cells (SRBC), ovalbumin or collagen) followed by administration of provided immunomodulatory proteins can be done to measure effect on B cell response.

Pharmacokinetic studies can be used in association with radiolabeled immunomodulatory proteins to determine the distribution and half life of such polypeptides in vivo.

In some embodiments, modeling and simulation of pharmacokinetic (PK) and pharmacodynamic (PD) profiles observed in control animals and animal models of disease (e.g., cancer models) can be used to predict or determine patient dosing. For example, PK data from non-human primates (e.g., cynomolgus monkeys) can be used to estimate human PK. Similarly, mouse PK and PD data can be used to predict human dosing. The observed animal data can be used to inform computational models which can be used to simulate human dose response.

### V. THERAPEUTIC APPLICATIONS

The pharmaceutical formulations described herein containing the TACI-Fc fusion protein can be used in a variety of therapeutic applications, such as in methods for the treatment of a disease. The therapeutic applications of the pharmaceutical formulations include methods and uses of any of the provided formulations. For example, in some embodiments the pharmaceutical formulation, such as any provided formulation, is used to treat inflammatory or autoimmune disorders, cancer, organ transplantation, viral infections, and/or bacterial infections in a mammal. The pharmaceutical formulation, such as any provided formulation, can modulate (e.g. decrease) an immune response to treat the disease.

Such methods and uses include therapeutic methods and uses, for example, involving administration of the formulations containing the same, to a subject having a disease, condition, or disorder. In some cases, such as described, the disease, condition or disorder is an autoimmune or inflammatory disease or disorder. In some embodiments, the formulation containing the TACI-Fc fusion protein is administered in an effective amount to effect treatment of the disease or disorder. Uses include uses of formulations containing a TACI-Fc fusion protein, and in the preparation of a medicament in order to carry out such therapeutic methods. In some embodiments, the methods are carried out by administering a formulation comprising the TACI-Fc fusion protein, to the subject having or suspected of having the disease or condition. In some embodiments, the methods thereby treat the disease, disorder or condition or disorder in the subject.

Illustrative subjects include mammalian subjects, such as farm animals, domestic animals, and human patients. In particular embodiments, the subject is a human subject.

The pharmaceutical formulations described herein can be used in a variety of therapeutic applications, such as the treatment of a disease. For example, in some embodiments the pharmaceutical formulation is used to treat inflammatory or autoimmune disorders, organ transplantation, viral infections, and/or bacterial infections in a mammal. The pharmaceutical formulation can modulate an immune response to treat the disease. In some embodiments, the pharmaceutical composition suppresses an immune response, which can be useful in the treatment of inflammatory or autoimmune disorders, or organ transplantation.

The provided methods are believed to have utility in a variety of applications, including, but not limited to, e.g., in prophylactic or therapeutic methods for treating a variety of immune system diseases or conditions in a mammal in which modulation or regulation of the immune system and immune system responses is beneficial. For example, suppressing an immune response can be beneficial in prophylactic and/or therapeutic methods for inhibiting rejection of a tissue, cell, or organ transplant from a donor by a recipient. In a therapeutic context, the mammalian subject is typically one with an immune system disease or condition, and administration is conducted to prevent further progression of the disease or condition.

### A. Diseases or Disorders for Treatment

The provided formulations containing a TACI-Fc fusion protein can be used for the treatment of autoimmune diseases, B cell cancers, immunomodulation, EBD and any antibody-mediated pathologies (e.g., ITCP, myasthenia gravis and the like), renal diseases, indirect T cell immune response, graft rejection, and graft versus host disease. The TACI-Fc fusion protein in some embodiments can specifically regulate B cell responses during the immune response. Additionally, administration of provided TACI-Fc fusion proteins can be used to modulate B cell development, development of other cells, antibody production, and cytokine production. Administration or use of provided TACI-Fc fusion proteins in some embodiments can also modulate B cell communication, such as by neutralizing the proliferative effects of BAFF or APRIL.

In some embodiments, the TACI-Fc fusion protein in the formulation suppresses an immune response, which can be useful in the treatment of inflammatory or autoimmune disorders, or organ transplantation. In some embodiments, the pharmaceutical formulation contains a TACI-Fc fusion protein that exhibits antagonist activity of a B cell stimulatory receptor, thereby decreasing or reducing an immune response. In some embodiments, the pharmaceutical formulations can be used to treat a B cell-mediated disease.

In some embodiments, the pharmaceutical formulations can be used to treat an autoimmune disease. In some embodiments, the administration of a pharmaceutical formulation containing an TACI-Fc fusion protein provided herein to a subject suffering from an immune system disease (e.g., autoimmune disease) can result in suppression or inhibition of such immune system attack or biological responses associated therewith. By suppressing this immune system attack on healthy body tissues, the resulting physical symptoms (e.g., pain, joint inflammation, joint swelling or tenderness) resulting from or associated with such attack on healthy tissues can be decreased or alleviated, and the biological and physical damage resulting from or associated with the immune system attack can be decreased, retarded, or stopped. In a prophylactic context, the subject may be one with, susceptible to, or believed to present an immune system disease, disorder or condition, and administration is typically conducted to prevent progression of the disease, disorder or condition, inhibit or alleviate symptoms, signs, or biological responses associated therewith, prevent bodily damage potentially resulting therefrom, and/or maintain or improve the subject's physical functioning.

In some embodiments, the disease or conditions that can be treated by the pharmaceutical formulation described herein is any disease mediated by immune complex deposition (e.g. lupus nephritis, vasculitis); direct interference with a pathway (e.g. catastrophic antiphospholipid antibody syndrome, myasthenia gravis crisis; anti-Jo-1 disease); opsonization or direct damage to cells (e.g. Idiopathic thrombocytopenic purpura, autoimmune hemolytic anemia); antibody-mediated rejection of an allograft (e.g. highly-sensitized renal transplant patients); or anti-drug antibodies to biologic replacement factors, vectors (e.g. anti-Factor 8).

In some embodiments, the inflammatory or autoimmune disorders, conditions or diseases that can be treated by the pharmaceutical formulation described herein is Systemic lupus erythematosus (SLE), including flare prevention without glucocorticoids; Sjögren's syndrome; Primary biliary cirrhosis (PBC); Systemic scleroderma; Polymyositis; Diabetes prevention; IgA nephropathy; IgA vasculitis; B cell cancers, for example myeloma; Multiple sclerosis, Optic neuritis.

In some embodiments, the inflammatory or autoimmune disorder is an inflammatory arthritis. Examples of inflammatory arthritis for treatment in accord with the provided methods include, but are not limited to rheumatoid arthritis, psoriatic arthritis, lupus, lyme disease, gout, or ankylosing spondylitis.

In some embodiments, the provided pharmaceutical formulations can be used to treat pre-B or B-cell leukemias, such as plasma cell leukemia, chronic or acute lymphocytic leukemia, myelomas such as multiple myeloma, plasma cell myeloma, endothelial myeloma and giant cell myeloma, and lymphomas such as non-Hodgkins lymphoma. In some of any embodiments, the type of myeloma includes multiple myeloma, plasmacytoma, multiple solitary plasmacytoma, and/or extramedullary myeloma. In some of any embodiments, the type of myeloma includes light chain myeloma, nonsecretory myeloma, and/or IgD or IgE myeloma.

In some embodiments, the provided pharmaceutical formulations can be used as immunosuppressants to selectively block the action of B-lymphocytes for use in treating disease. For example, certain autoimmune diseases are characterized by production of autoantibodies, which contribute to tissue destruction and exacerbation of disease. Autoantibodies can also lead to the occurrence of immune complex deposition complications and lead to many symptoms of systemic lupus erythematosus, including kidney failure, neuralgic symptoms and death. Modulating antibody production independent of cellular response would also be beneficial in many disease states. B cells have also been shown to play a role in the secretion of arthritogenic immunoglobulins in rheumatoid arthritis. Methods and uses of the provided immunomodulatory proteins to inhibit, block or neutralize action of B cells to thereby suppress antibody production would be beneficial in treatment of autoimmune diseases such as myasthenia gravis, rheumatoid arthritis, polyarticular-course juvenile rheumatoid arthritis, and psoriatic arthritis.

In some embodiments, the provided pharmaceutical formulations can be used to block or neutralize the actions of B-cells in association with end stage renal diseases, which may or may not be associated with autoimmune diseases. Such methods would also be useful for treating immunologic renal diseases. Such methods would be useful for treating glomerulonephritis associated with diseases such as membranous nephropathy, IgA nephropathy or Berger's Disease, IgM nephropathy, IgA Vasculitis, Goodpasture's Disease, post-infectious glomerulonephritis, mesangioproliferative disease, chronic lymphoid leukemia, minimal-change nephrotic syndrome. Such methods would also serve as therapeutic applications for treating secondary glomerulonephritis or vasculitis associated with such diseases as lupus, polyarteritis, Henoch-Schonlein, Scleroderma, HTV-related diseases, amyloidosis or hemolytic uremic syndrome. The provided methods would also be useful as part of a therapeutic application for treating interstitial nephritis or pyelonephritis associated with chronic pyelonephritis, analgesic abuse, nephrocalcinosis, nephropathy caused by other agents, nephrolithiasis, or chronic or acute interstitial nephritis. The methods provided herein also include use of the provided immunomodulatory proteins in the treatment of hypertensive or large vessel diseases, including renal artery stenosis or occlusion and cholesterol emboli or renal emboli. The provided methods and uses also can be used for treatment of renal or urological neoplasms, multiple myelomas, lymphomas, light chain neuropathy or amyloidosis.

In some embodiments, the provided pharmaceutical formulations also can be used for the treatment of asthma and other chronic airway diseases such as bronchitis and emphysema. The provided immunomodulatory proteins can also be used to treat Sjogren's Syndrome.

In some embodiments, methods and uses of the provided pharmaceutical formulations include immunosuppression, in particular for such therapeutic use as for graft-versus-host disease and graft rejection. In some embodiments, methods and uses of the provided immunomodulatory proteins include treatment of such autoimmune diseases as insulin dependent diabetes mellitus (IDDM) and Crohn's Disease. Methods provided herein would have additional therapeutic value for treating chronic inflammatory diseases, in particular to lessen joint pain, swelling, anemia and other associated symptoms as well as treating septic shock.

In some embodiments, the pharmaceutical formulations can be used to treat an autoantibody-related disease or disorder.

In some embodiments, the autoantibody-related disease or disorder treated by a pharmaceutical formulation containing an immunomodulatory protein described herein includes, but is not limited to, a rheumatic disease or disorder, a hematologic disease or disorder, a dermatologic disease or disorder, or a neurologic disease or disorder. In some embodiments, the autoantibody-related disease or disorder comprises cytopenia, pemphigus foliaceus, blistering disease or encephalitis. In some embodiments, the encephalitis comprises limbic encephalitis.

In some embodiments, the inflammatory and autoimmune disorders that can be treated by a pharmaceutical formulation containing an immunomodulatory protein described herein include, but are not limited to, Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune adrenalitis (Addison's disease); Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune polyglandular syndrome type II (APS II); Autoimmune retinopathy; Autoimmune thyroid disease (AITD), i.e. Hashimoto's disease; Autoimmune urticarial; Axonal & neuronal neuropathy (AMAN); Bal6 disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy (IgAN); IgA Vasculitis; IgG4-related sclerosing disease; Immune thrombocytopenia; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica (NMO); Neuromyelitis optica spectrum disorder (NMOSD); Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus, Pemphigus vulgaris; Peripheral neuropathy; Perivenous encephalomyelitis; Pernicious anemia (PA); POEMS syndrome; Polyarteritis nodosa; Polyglandular syndromes type I, II, III; Polymyalgia rheumatic; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); systemic lupus erythematosus (SLE); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo or Vogt-Koyanagi-Harada Disease. In some embodiments, the provided immunomodulatory proteins (e.g. TACI-Fc) can be used to treat Scleroderma, Myasthenia gravis, GVHD (including acute GVHD or chronic GVHD), an immune response in connection with transplantation; Antiphospholipid Ab syndrome; Multiple sclerosis; Sjogren's syndrome; IgG4-related disease; Type I diabetes; Rheumatoid arthritis including glucocorticoid therapy (GC) RA or Acute lupus nephritis.

In some embodiments, the provided pharmaceutical formulations can be used to treat Amyotrophic lateral sclerosis, Neuromyelitis optica (NMO), Neuromyelitis optica spectrum disorder (NMOSD), Transverse myelitis, CNS autoimmunity, Guillain-barre syndrome, Neurocystercercosis, Sarcoidosis (T/seroneg), Churg-Strauss Syndrome, Hashimoto's thyroiditis, Grave's disease, immune thrombocytopenia (ITP), Addison's Disease, Polymyositis, or Dermatomyositis.

In some embodiments, the provided pharmaceutical formulations can be used to treat IgA nephropathy, chronic inflammatory demyelinating polyneuropathy (CIDP), antisynthetase disease such as Jo-1 syndrome, or ANCA vasculitis.

In some embodiments, the provided pharmaceutical formulations can be used to treat an autoantibody-associated glomerular disease. In some embodiments, the autoantibody-associated glomerular disease may include immunoglobulin (Ig) A nephropathy (IgAN), lupus nephritis (LN), membranous nephropathy, such as primary membranous nephropathy (pMN), or renal anti-neutrophil cytoplasmic antibody (ANCA)-associated vasculitis (AAV). In some embodiments, the provided pharmaceutical compositions comprising a TACI-Fc fusion protein can be used to treat IgAN.

In some embodiments, the provided pharmaceutical formulations can be used to treat a cytopenia, such as Immune Thrombocytopenia, Immune Thrombocytopenic Purpura, Idiopathic Thrombocytopenic Purpura, Cold Autoimmune Hemolytic Anemia, Warm Autoimmune Hemolytic Anemia, or Autoimmune Hemolytic Anemia.

In some embodiments, the provided immunomodulatory proteins (e.g. TACI-Fc) can be used to treat systemic lupus erythematosus (SLE).

In some embodiments, the provided immunomodulatory proteins (e.g. TACI-Fc) can be used to treat Sjogren's syndrome (SjS).

In some embodiments, the provided immunomodulatory proteins (e.g. TACI-Fc) can be used to treat myasthenia gravis.

In some embodiments, the provided pharmaceutical formulations comprising a TACI-Fc fusion protein can be used to treat autoimmune bullous (blistering) dermatosis (ABDs; also known as autoimmune blistering diseases). In some embodiments, the provided pharmaceutical formulations comprising a TACI-Fc fusion protein can be used to treat pemphigus vulgaris, pemphigus foliaceus, or epidermolysis bullosa acquisita (EBA).

In some embodiments, the provided pharmaceutical formulations comprising a TACI-Fc fusion protein can be used to treat autoimmune encephalitis or limbic encephalitis.

In some embodiments, the provided immunomodulatory proteins (e.g. TACI-Fc) can be used to treat autoimmune myasthenia gravis.

In some embodiments, the provided pharmaceutical formulations can be used to treat a B cell cancer. In some embodiments, the B cell cancer is a cancer in which BAFF and APRIL are involved or implicated in providing an autocrine survival loop to the B cells. In some embodiments, the cancer is B cell chronic lymphocytic leukemia, non-Hodgkins' lymphoma or myeloma. In some embodiments, the cancer is myeloma. In some of any embodiments, the type of myeloma includes multiple myeloma, plasmacytoma, multiple solitary plasmacytoma, and/or extramedullary myeloma. In some of any embodiments, the type of myeloma includes light chain myeloma, nonsecretory myeloma, and/or IgD or IgE myeloma.

In some embodiments, the subject may receive standard of care (SOC) therapy for their underlying disorder, which is within the level of skill of the investigator or clinical physician. In some embodiments, the subject has previously received the SOC therapy prior to receiving the provided TACI-Fc fusion protein. In some embodiments, the subject continues receiving the SOC therapy while receiving administration of the provided TACI-Fc fusion protein. In some embodiments, the SOC therapy is tapered over time after receiving administration of the provided TACI-Fc fusion protein.

In some embodiments, the subject has not received an agent that directly depletes B lymphocytes (e.g. Rituximab) within 48 weeks prior to initiation of administration of the provided immunomodulatory proteins (e.g. TACI-Fc). In some embodiments, the subject may have received an agent that directly depletes B lymphocytes (e.g. Rituximab) within greater than 24 weeks prior to initiation of administration of the provided immunomodulatory proteins (e.g. TACI-Fc) if B cells have returned to normal reference ranges prior to administration of the TACI-Fc fusion protein.

In some embodiments, the subject has not received an agent that directly inhibits BAFF and/or APRIL, such as Belimumab, within 24 weeks prior to initiation of administration of the provided immunomodulatory proteins (e.g. TACI-Fc).

In some embodiments, the subject has not received administration of Intravenous Ig, abatacept, anifrolumab, belatacept, adalimumab, infliximab, certolizumab, etanercept, golimumab, anakinra, canakinumab, tocilizumab, sarilumab, satralizumab within 8 weeks prior to initiation of administration of the provided immunomodulatory proteins (e.g. TACI-Fc). In some embodiments, the subject has not received administration of any approved therapeutic agent for treating an immune disease within 8 weeks prior to initiation of administration of the provided immunomodulatory proteins (e.g. TACI-Fc).

In some embodiments, the subject has not received cyclophosphamide within 8 weeks prior to initiation of administration of the provided immunomodulatory proteins (e.g. TACI-Fc).

### B. Dosing and Administration

In some embodiments, the TACI-Fc formulation is administered to a subject. Generally, dosages and routes of administration of the pharmaceutical formulation are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

Typically, a precise amount of the pharmaceutical formulations of the present invention to be administered can be determined by a physician with consideration of individual differences in age, weight, extent of infection, and condition of the patient (subject). The optimal dosage and treatment regimen for a particular patient can readily be determined by one skilled in the art of medicine by monitoring the patient for signs of disease and adjusting the treatment accordingly.

In some embodiments, the subject is human. In some embodiments, the subject is an adult subject. In some embodiments, the subject is greater than or equal to 18 years of age.

In some embodiments, a pharmaceutical formulation described herein (including a pharmaceutical formulation any of the TACI-Fc fusion proteins described herein) is administered to a subject. Generally, dosages and routes of administration of the pharmaceutical formulation are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage can be determined in light of factors related to the subject requiring treatment. Dosage and administration can be adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

In some embodiments, modeling and simulation of pharmacokinetic (PK) and pharmacodynamic (PD) profiles observed in control animals and animal models of disease (e.g., cancer models) can be used to predict or determine patient dosing. For example, PK data from non-human primates (e.g., cynomolgus monkeys) can be used to estimate human PK. Similarly, mouse or rat PK and PD data can be used to predict human dosing. The observed animal data can be used to inform computational models which can be used to simulate human dose response.

In some embodiments, methods provided herein include administering a pharmaceutical formulation comprising a TACI-Fc fusion protein in an amount in which a dose is known or predicted to neutralize an activity of APRIL or BAFF ligand, including a BAFF or APRIL homotrimer, a BAFF/APRIL heterotimer or a BAFF 60mer, sufficient for a therapeutic effect. The particular amount can be determined experimentally or empirically. In some embodiments, the amount can be empirically determined from in vitro binding data or from animal models.

In some embodiments, the TACI-Fc fusion protein, or pharmaceutical formulations thereof, may be administered every 3 to 4 days, once every week, biweekly, every three weeks, every four weeks, once every eight weeks or once every twelve weeks. The precise timing and frequency can be empirically determined by a skilled clinician or physician, such as depending on the particular half-life and clearance rate of the particular formulation. The frequency of dosing will depend upon the pharmacokinetic parameters of the molecule in the formulation used. Typically, a formulation is administered until a dosage is reached that achieves the desired effect. The formulation may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some cases, for example when a chronic inflammatory or autoimmune disorder is treated with a pharmaceutical formulation provided herein, such as a TACI-Fc fusion protein provided herein, the formulation is administered continuously, e.g., repeatedly, over time or intermittently over time. The duration of administration can be for weeks, months or years. In some cases, treatment of a chronic inflammatory or autoimmune disorder, e.g., with a pharmaceutical formulation provided herein, such as a containing a TACI-Fc fusion protein provided herein, may include administering the treatment to a subject indefinitely. In some embodiments, when the inflammatory or autoimmune disorder is a chronic inflammatory or autoimmune disorder, treatment with a pharmaceutical formulation provided herein, such as a TACI-Fc fusion protein provided herein, is continued following remission or partial remission of the disease and/or a reduction or amelioration in signs and/or symptoms of a disease, such as a reduction of one or more signs of inflammation in a subject having the chronic inflammatory or autoimmune disorder. In some embodiments, administration continues until any time as desired by a skilled practitioner. In some cases, for example when an acute inflammatory or autoimmune disorder is treated with a pharmaceutical formulation provided herein, such as a TACI-Fc fusion protein provided herein, the formulation is administered for a defined or limited period of time. In some embodiments, when the inflammatory or autoimmune disorder is an acute inflammatory or autoimmune disorder, treatment with a pharmaceutical formulation provided herein, such as a TACI-Fc fusion protein provided herein, is discontinued following remission or partial remission of the disease and/or a reduction or amelioration in signs and/or symptoms of a disease, such as a reduction of one or more signs of inflammation in a subject having the acute inflammatory or autoimmune disorder. In some embodiments, administration is discontinued at any time as desired by a skilled practitioner.

Typically, precise amount of the formulations of the present invention to be administered can be determined by a physician with consideration of individual differences in age, weight, tumor size, extent of infection or metastasis, and condition of the patient (subject). In some embodiments, when referencing dosage based on mg/kg of the subject, an average human subject is considered to have a mass of about 70 kg-75 kg, such as 70 kg and a body surface area (BSA) of 1.73 m².

In some embodiments, the dosage of the pharmaceutical formulation is a single dose or a repeated dose. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, multiple doses are given over the course of days, weeks, months, or years. In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In particular embodiments, a TACI-Fc fusion protein is administered as a plurality of doses where each dose is administered no more than once weekly. In some embodiments, each does is administered once a week (Q1W). In some embodiments, each dose is administered once every two weeks (Q2W). In some embodiments, each dose is administered once every three weeks (Q3W). In some embodiments, each dose is administered once every four weeks (Q4W). In some embodiments, each dose is administered once every two months (e.g. Q8W). In some embodiments, each dose is administered once every three months (e.g. Q12W). In some embodiments, each dose is administered once every 6 months (e.g., Q24W). In aspects of provided embodiments, the administration cycle is repeated a plurality of times to administer a plurality of doses of the TACI-Fc fusion protein. In some embodiments, the administration is continued for a predetermined period of time, e.g. 4 weeks, 6 weeks, 8 weeks, 3 months, 6 months, 1 year or more. In some embodiments, the administration is discontinued after relapse or progression of the disease or condition in the subject.

In some embodiments, a dose regimen as described herein is administered to achieve a therapeutically effective amount to treat the disease, disorder or condition in the subject in need thereof. In some embodiments, each dose of the TACI-Fc fusion protein is administered in an amount between at or about 2.4 mg and at or about 960 mg, inclusive. In some embodiments, each dose of the TACI-Fc fusion protein is administered in an amount between at or about 8 mg and at or about 960 mg, between at or about 8 mg and at or about 880 mg, between at or about 8 mg and at or about 800 mg, between at or about 8 mg and at or about 720 mg, between at or about 8 mg and at or about 640 mg, between at or about 8 mg and at or about 560 mg, between at or about 8 mg and at or about 480 mg, between at or about 8 mg and at or about 400 mg, between at or about 8 mg and at or about 320 mg, between at or about 8 mg and at or about 240 mg, between at or about 8 mg and at or about 160 mg, between at or about 8 mg and at or about 80 mg, between at or about 8 mg and at or about 40 mg, between at or about 40 mg and at or about 960 mg, between at or about 40 mg and at or about 880 mg, between at or about 40 mg and at or about 800 mg, between at or about 40 mg and at or about 720 mg, between at or about 40 mg and at or about 640 mg, between at or about 40 mg and at or about 560 mg, between at or about 40 mg and at or about 480 mg, between at or about 40 mg and at or about 400 mg, between at or about 40 mg and at or about 320 mg, between at or about 40 mg and at or about 240 mg, between at or about 40 mg and at or about 160 mg, between at or about 40 mg and at or about 80 mg, between at or about 80 mg and at or about 960 mg, between at or about 80 mg and at or about 880 mg, between at or about 80 mg and at or about 800 mg, between at or about 80 mg and at or about 720 mg, between at or about 80 mg and at or about 640 mg, between at or about 80 mg and at or about 560 mg, between at or about 80 mg and at or about 480 mg, between at or about 80 mg and at or about 400 mg, between at or about 80 mg and at or about 320 mg, between at or about 80 mg and at or about 240 mg, between at or about 80 mg and at or about 160 mg, between at or about 160 mg and at or about 960 mg, between at or about 160 mg and at or about 880 mg, between at or about 160 mg and at or about 800 mg, between at or about 160 mg and at or about 720 mg, between at or about 160 mg and at or about 640 mg, between at or about 160 mg and at or about 560 mg, between at or about 160 mg and at or about 480 mg, between at or about 160 mg and at or about 400 mg, between at or about 160 mg and at or about 320 mg, between at or about 160 mg and at or about 240 mg, between at or about 240 mg and at or about 960 mg, between at or about 240 mg and at or about 880 mg, between at or about 240 mg and at or about 800 mg, between at or about 240 mg and at or about 720 mg, between at or about 240 mg and at or about 640 mg, between at or about 240 mg and at or about 560 mg, between at or about 240 mg and at or about 480 mg, between at or about 240 mg and at or about 400 mg, between at or about 240 mg and at or about 320 mg, between at or about 320 mg and at or about 960 mg, between at or about 320 mg and at or about 880 mg, between at or about 320 mg and at or about 800 mg, between at or about 320 mg and at or about 720 mg, between at or about 320 mg and at or about 640 mg, between at or about 320 mg and at or about 560 mg, between at or about 320 mg and at or about 480 mg, between at or about 320 mg and at or about 400 mg, between at or about 400 mg and at or about 960 mg, between at or about 400 mg and at or about 880 mg, between at or about 400 mg and at or about 800 mg, between at or about 400 mg and at or about 720 mg, between at or about 400 mg and at or about 640 mg, between at or about 400 mg and at or about 560 mg, between at or about 400 mg and at or about 480 mg, between at or about 480 mg and at or about 960 mg, between at or about 480 mg and at or about 880 mg, between at or about 480 mg and at or about 800 mg, between at or about 480 mg and at or about 720 mg, between at or about 480 mg and at or about 640 mg, between at or about 480 mg and at or about 560 mg, between at or about 560 mg and at or about 960 mg, between at or about 560 mg and at or about 880 mg, between at or about 560 mg and at or about 800 mg, between at or about 560 mg and at or about 720 mg, between at or about 560 mg and at or about 640 mg, between at or about 640 mg and at or about 960 mg, between at or about 640 mg and at or about 880 mg, between at or about 640 mg and at or about 800 mg, between at or about 640 mg and at or about 720 mg, between at or about 720 mg and at or about 960 mg, between at or about 720 mg and at or about 880 mg, between at or about 720 mg and at or about 800 mg, between at or about 800 mg at or about 960 mg, between at or about 800 mg and at or about 880 mg or between at or about 880 mg and at or about 960 mg, each inclusive.

In some embodiments, each dose of the TACI-Fc fusion protein is administered in an amount between at or about 8 mg and at or about 20 mg, between at or about 20 mg and at or about 960 mg, between at or about 20 mg and at or about 880 mg, between at or about 20 mg and at or about 800 mg, between at or about 20 mg and at or about 720 mg, between at or about 20 mg and at or about 640 mg, between at or about 20 mg and at or about 560 mg, between at or about 20 mg and at or about 480 mg, between at or about 20 mg and at or about 400 mg, between at or about 20 mg and at or about 320 mg, between at or about 20 mg and at or about 240 mg, between at or about 20 mg and at or about 160 mg, between at or about 20 mg and at or about 40 mg, each inclusive.

In some embodiments, each dose of the TACI-Fc fusion protein is administered in an amount between at or about 8 mg and at or about 20 mg, between at or about 20 mg and at or about 960 mg, between at or about 20 mg and at or about 880 mg, between at or about 20 mg and at or about 800 mg, between at or about 20 mg and at or about 720 mg, between at or about 20 mg and at or about 640 mg, between at or about 20 mg and at or about 560 mg, between at or about 20 mg and at or about 480 mg, between at or about 20 mg and at or about 400 mg, between at or about 20 mg and at or about 320 mg, between at or about 20 mg and at or about 240 mg, between at or about 20 mg and at or about 160 mg, between at or about 20 mg and at or about 40 mg, each inclusive.

In some embodiments, each dose of a TACI-Fc fusion protein is or is about 2.4 mg. In some embodiments, each dose of a TACI-Fc fusion protein is or is about 8 mg. In some embodiments, each dose of a TACI-Fc fusion protein is or is about 20 mg. In some embodiments, each dose of a TACI-Fc fusion protein is or is about 24 mg. In some embodiments, each dose of a TACI-Fc fusion protein is or is about 40 mg. In some embodiments, each dose of a TACI-Fc fusion protein is or is about 80 mg. In some embodiments, each dose of a TACI-Fc fusion protein is or is about 160 mg. In some embodiments, each dose of a TACI-Fc fusion protein is or is about 240 mg. In some embodiments, each dose of a TACI-Fc fusion protein is or is about 320 mg. In some embodiments, each dose of a TACI-Fc fusion protein is or is about 400 mg. In some embodiments, each dose of a TACI-Fc fusion protein is or is about 480 mg. In some embodiments, each dose of a TACI-Fc fusion protein is or is about 560 mg. In some embodiments, each dose of a TACI-Fc fusion protein is or is about 640 mg. In some embodiments, each dose of a TACI-Fc fusion protein is or is about 720 mg. In some embodiments, each dose of a TACI-Fc fusion protein is or is about 800 mg. In some embodiments, each dose of a TACI-Fc fusion protein is or is about 880 mg. In some embodiments, each dose of a TACI-Fc fusion protein is or is about 960 mg.

In some embodiments, the dose is an amount between or between about 40 mg and at or about 480 mg, between at or about 80 mg to at or about 320 mg, or between at or at or about 80 mg to at or about 120 mg, each inclusive.

In some embodiments, each dose of the TACI-Fc fusion protein is administered once every three months. In some embodiments, the TACI-Fc fusion protein is administered in an amount from at or about 160 mg to at or about 960 mg once every three months. In some embodiments, the TACI-Fc fusion protein is administered in an amount from at or about 240 mg to at or about 800 mg once every three months. In some embodiments, the TACI-Fc fusion protein is administered in an amount from at or about 480 mg to at or about 720 mg once every three months.

In some embodiments, each dose of the TACI-Fc fusion protein is administered once every month (once every four weeks or Q4W). In some embodiments, the TACI-Fc fusion protein is administered in an amount from at or about 2.4 mg to at or about 960 mg Q4W. In some embodiments, the TACI-Fc fusion protein is administered in an amount from at or about 80 mg to at or about 720 mg Q4W. In some embodiments, the TACI-Fc fusion protein is administered in an amount from at or about 160 mg to at or about 560 mg Q4W. In some embodiments, the TACI-Fc fusion protein is administered in an amount from at or about 240 mg to at or about 480 mg Q4W. In some embodiments, the TACI-Fc fusion protein is administered at or about 24 mg Q4W. In some embodiments, the TACI-Fc fusion protein is administered at or about 80 mg Q4W. In some embodiments, the TACI-Fc fusion protein is administered at or about 160 mg Q4W. In some embodiments, the TACI-Fc fusion protein is administered at or about 240 mg Q4W. In some embodiments, the TACI-Fc fusion protein is administered subcutaneously. In some embodiments, the TACI-Fc fusion protein is administered intravenously.

In some embodiments, each dose of the TACI-Fc fusion protein is administered once every other week (Q2W). In some embodiments, the TACI-Fc fusion protein is administered in an amount from at or about 2.4 mg to at or about 960 mg Q2W. In some embodiments, the TACI-Fc fusion protein is administered in an amount from at or about 80 mg to at or about 720 mg Q2W. In some embodiments, the TACI-Fc fusion protein is administered in an amount from at or about 160 mg to at or about 560 mg Q2W. In some embodiments, the TACI-Fc fusion protein is administered in an amount from at or about 240 mg to at or about 480 mg Q2W. In some embodiments, the TACI-Fc fusion protein is administered at or about 80 mg Q2W. In some embodiments, the TACI-Fc fusion protein is administered at or about 160 mg Q2W. In some embodiments, the TACI-Fc fusion protein is administered at or about 240 mg Q2W. In some embodiments, the TACI-Fc fusion protein is administered subcutaneously. In some embodiments, the TACI-Fc fusion protein is administered intravenously.

In some embodiments, each dose of the TACI-Fc fusion protein is administered once every two months (Q8W). In some embodiments, the TACI-Fc fusion protein is administered in an amount from at or about 2.4 mg to at or about 960 mg Q8W. In some embodiments, the TACI-Fc fusion protein is administered in an amount from at or about 80 mg to at or about 720 mg Q8W. In some embodiments, the TACI-Fc fusion protein is administered in an amount from at or about 160 mg to at or about 560 mg Q8W. In some embodiments, the TACI-Fc fusion protein is administered in an amount from at or about 240 mg to at or about 480 mg Q8W. In some embodiments, the TACI-Fc fusion protein is administered at or about 24 mg Q8W. In some embodiments, the TACI-Fc fusion protein is administered at or about 80 mg Q8W. In some embodiments, the TACI-Fc fusion protein is administered at or about 160 mg Q8W. In some embodiments, the TACI-Fc fusion protein is administered at or about 240 mg Q8W. In some embodiments, the TACI-Fc fusion protein is administered at or about 320 mg Q8W. In some embodiments, the TACI-Fc fusion protein is administered at or about 480 mg Q8W. In some embodiments, the TACI-Fc fusion protein is administered subcutaneously. In some embodiments, the TACI-Fc fusion protein is administered intravenously.

In some embodiments, each dose of the TACI-Fc fusion protein is administered once every three months (Q12W). In some embodiments, the TACI-Fc fusion protein is administered in an amount from at or about 2.4 mg to at or about 960 mg Q12W. In some embodiments, the TACI-Fc fusion protein is administered in an amount from at or about 80 mg to at or about 720 mg Q12W. In some embodiments, the TACI-Fc fusion protein is administered in an amount from at or about 160 mg to at or about 560 mg Q12W. In some embodiments, the TACI-Fc fusion protein is administered in an amount from at or about 240 mg to at or about 480 mg Q12W. In some embodiments, the TACI-Fc fusion protein is administered at or about 24 mg Q12W. In some embodiments, the TACI-Fc fusion protein is administered at or about 80 mg Q12W. In some embodiments, the TACI-Fc fusion protein is administered at or about 160 mg Q12W. In some embodiments, the TACI-Fc fusion protein is administered at or about 240 mg Q12W. In some embodiments, the TACI-Fc fusion protein is administered subcutaneously. In some embodiments, the TACI-Fc fusion protein is administered intravenously.

In some embodiments, each dose of the TACI-Fc fusion protein is administered once a week (Q1W). In some embodiments, the TACI-Fc fusion protein is administered in an amount from at or about 2.4 mg to at or about 960 mg Q1W. In some embodiments, the TACI-Fc fusion protein is administered in an amount from at or about 40 mg to at or about 480 mg Q1W. In some embodiments, the TACI-Fc fusion protein is administered in an amount from at or about 80 mg to at or about 320 mg Q1W. In some embodiments, the TACI-Fc fusion protein is administered in an amount from at or about 80 mg and at or about 120 mg Q1W.

In some embodiments, each dose of the TACI-Fc fusion protein is administered once every six months (Q24W). In some embodiments, the TACI-Fc fusion protein is administered in an amount from at or about 2.4 mg to at or about 960 mg Q24W. In some embodiments, the TACI-Fc fusion protein is administered in an amount from at or about 80 mg to at or about 720 mg Q24W. In some embodiments, the TACI-Fc fusion protein is administered in an amount from at or about 160 mg to at or about 560 mg Q24W. In some embodiments, the TACI-Fc fusion protein is administered in an amount from at or about 240 mg to at or about 480 mg Q24W. In some embodiments, the TACI-Fc fusion protein is administered at or about 24 mg Q24W. In some embodiments, the TACI-Fc fusion protein is administered at or about 80 mg Q24W. In some embodiments, the TACI-Fc fusion protein is administered at or about 160 mg Q24W. In some embodiments, the TACI-Fc fusion protein is administered at or about 240 mg Q24W. In some embodiments, the TACI-Fc fusion protein is administered subcutaneously. In some embodiments, the TACI-Fc fusion protein is administered intravenously.

It is contemplated that dosing (e.g., multiple doses), can continue until any time as desired by a skilled practitioner. For example, dosing may continue until a desirable disease response is achieved, such as in remission or partial remission of the disease and/or a reduction or amelioration in signs and/or symptoms of a disease, such as a reduction of one or more signs of inflammation in the subject. In some embodiments, the dosing is continued following remission or partial remission of the disease and/or a reduction or amelioration in signs and/or symptoms of a disease, such as a reduction of one or more signs of inflammation in the subject.

The administration of the subject formulations may be carried out in any convenient manner, including by aerosol inhalation, injection, ingestion, transfusion, implantation or transplantation. The formulations described herein may be administered to a patient subcutaneously, intradermally, intratumorally, intranodally, intramedullary, intramuscularly, by intravenous (i.v.) injection, or intraperitoneally. In one embodiment, the therapeutic formulation is administered to a patient by intradermal or subcutaneous injection. In another embodiment, the therapeutic formulation is administered by i.v. injection.

In some embodiments, a provided pharmaceutical formulation may, for example, be in a form suitable for intravenous infusion. In some embodiments, a provided formulation may be in in a form suitable for subcutaneous administration.

In some embodiments, the pharmaceutical formulation comprising any of the TACI-Fc fusion proteins described herein is administered to a subject via subcutaneous administrations. In some embodiments, the dose of the TACI-Fc fusion for subcutaneous administration is at or about 80 mg. In some embodiments, the dose of the TACI-Fc fusion for subcutaneous administration is at or about 240 mg. In some embodiments, the dose of the TACI-Fc fusion for subcutaneous administration is at or about 480 mg. In some embodiments, the dose of the TACI-Fc fusion for subcutaneous administration is at or about 720 mg. In some embodiments, each dose is administered subcutaneously Q1W. In some embodiments, each dose is administered subcutaneously Q2W. In some embodiments, each dose is administered subcutaneously Q4W (i.e. once a month).

In some embodiments, the pharmaceutical formulation comprising any of the TACI-Fc fusion proteins described herein is administered to a subject via intravenous administration. In some embodiments, the dose of the TACI-Fc fusion for intravenous administration is at or about 2.4 mg. In some embodiments, the dose of the TACI-Fc fusion for intravenous administration is at or about 8 mg. In some embodiments, the dose of the TACI-Fc fusion for intravenous administration is at or about 24 mg. In some embodiments, the dose of the TACI-Fc fusion for intravenous administration is at or about 80 mg. In some embodiments, the dose of the TACI-Fc fusion for intravenous administration is at or about 240 mg. In some embodiments, the dose of the TACI-Fc fusion for intravenous administration is at or about 480 mg. In some embodiments, the dose of the TACI-Fc fusion for intravenous administration is at or about 720 mg. In some embodiments, each dose is administered intravenously Q1W. In some embodiments, each dose is administered intravenously Q2W. In some embodiments, each dose is administered intravenously Q4W (i.e. once every 4 weeks).

In some embodiments, the pharmaceutical formulation comprising any of the TACI-Fc fusion proteins described herein is administered parenterally. In some embodiments, the pharmaceutical formulation is in a form suitable for infusion injection, for example by intravenous injection. In some embodiments, the infusion duration is, is at least, or is about 30 minutes, 40 minutes, 50 minutes, 1 hour, 1.5 hours, 2 hours, 3 hours, 4 hours, 5 hours or 6 hours. In some embodiments the infusion duration is between about 30 minutes and 6 hours. In some embodiments, the infusion duration is between about 30 minutes and 5 hours. In some embodiments, the infusion duration is between about 30 minutes and 4 hours. In some embodiments, the infusion duration is between about 30 minutes and 3 hours. In some embodiments, the infusion duration is between about 30 minutes and 2 hours. In some embodiments, the infusion duration is between about 30 minutes and 1 hour. In some embodiments, the infusion duration is or is about 30 minutes.

In some embodiments, a dosing regimen may include intravenous and subcutaneous dosing. In some embodiments, an initial loading dose may be administered intravenously, followed by a maintenance dose(s) administered subcutaneously. In some embodiments, there is provided a load/maintenance regimen, in which an intravenous dose is given one time, followed by a subcutaneous dose on the same day, and then followed by administration of administration of maintenance doses subcutaneously once a week to once every three weeks. In some embodiments, the maintenance dose is administered once a week (Q1W). In some embodiments, the maintenance dose is administered once every two weeks (Q2W). In some embodiments, the maintenance dose is administered once every four weeks (Q4W). In some embodiments, the maintenance dose is administered once every twelve weeks (Q12W).

In some embodiments, the dosing regimen may also include an intermediate/step down regimen in which the dose amount and/or frequency of administration is reduced over time. In some embodiment, the TACI-Fc fusion protein is administered once a week (Q1W) for four weeks, and then is administered once a month (Q4W). In some embodiments, the TACI-Fc fusion protein is administered once a week (Q1W) for four weeks, and then is administered once a week (Q1W) or once every two weeks (Q2W) for two to four weeks, and then is administered once every twelve weeks (Q12W).

In some embodiments, the dosing regimen may also include an intermediate/step down regimen in which the dose amount and/or frequency of administration is reduced over time. In some embodiment, the TACI-Fc fusion protein is administered once a week (Q1W) for three to four doses, and then is administered once every four weeks (Q4W). In some embodiments, the TACI-Fc fusion protein is administered once a week (Q1W) for three four doses, and then is administered once a week (Q1W) or once every two weeks (Q2W) for two to four weeks, and then is administered once every three months (Q12W). In some embodiment, the TACI-Fc fusion protein is administered once every other week (Q2W) for three to four doses, and then is administered once every four weeks (Q4W). For instance, in some embodiments, the Q1W or Q2W dose is given for 3-4 doses then monthly (Q4W) at that dose or a higher dose. In some embodiments, at or about 80 mg is administered Q1W or Q2W for 3-4 doses and then monthly (Q4W) at that dose or a higher dose (e.g. 160 mg or 240 mg).

In some embodiments, the administration of the provided TACI-Fc fusion protein in accord with the provided methods continues for a desired time as determined by a treating physician or investigator. In some embodiments, the administration is continued until the subject exhibits a complete response or clinical remission. In some embodiments, the administration of the provided TACI-Fc fusion protein in accord with the provided methods continues for a treatment period. In some embodiments, the treatment period is for at or about 6 months to 3 years, such as at or about 24 weeks, 36 weeks, 48 weeks, 1 year (e.g. 52 weeks), 2 years or 3 years. In some embodiments, the administration is continued until such time as the subject's symptoms are worsening or the disease or condition has progressed or relapsed in the subject following a remission.

In some embodiments, the pharmaceutical formulation is administered as a monotherapy (i.e., as a single agent) or as a combination therapy (i.e., in combination with one or more additional immunosuppressant agents). In some embodiments, the additional agent is a glucocorticoid (e.g., prednisone, dexamethasone, and hydrocortisone), cytostatic agent, such as a cytostatic agent that affect proliferation of T cells and/or B cells (e.g., purine analogs, alkylating agents, or antimetabolites), an antibody (e.g., anti-CD20, anti-CD25 or anti-CD3 monoclonal antibodies), cyclosporine, tacrolimus, sirolimus, everolimus, an interferon, an opioid, a TNF binding protein, mycophenolate, small biological agent, such as fingolimod or myriocin, cytokine, such as interferon beta-la, an integrin agonist, or an integrin antagonist.

In some embodiments, the efficacy of the treatment is monitored in the subject. In some embodiments, the change in baseline over time in circulating levels of antibodies, such as autoantibodies are monitored in the subject.

In some embodiments, a clinical response is monitored in the subject.

In some embodiments of the methods, the treating can result in a clinical remission. In some aspects, the treating can result in a clinical remission without about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in a clinical remission within about 10 weeks from the first dose. In some embodiments, the treating results in a clinical remission within about 6 weeks from the first dose. In some embodiments, the treating results in a clinical remission at about 6 weeks from the first dose and at about 10 weeks from the first dose.

In some embodiments of any of the preceding methods, the clinical remission is a sustained remission. For example, in some embodiments, the sustained remission is a clinical remission at about 10 weeks, about 15 weeks, about 20 weeks, about 25 weeks, about 30 weeks, about 35 weeks, about 40 weeks, about 45 weeks, about 50 weeks, about 52 weeks, about 55 weeks, about 60 weeks, about 65 weeks, about 70 weeks, about 72 weeks, about 75 weeks, about 80 weeks, about 85 weeks, about 90 weeks, about 95 weeks, about 100 weeks, about 102 weeks, about 105 weeks, or about 110 weeks from the first dose. In some embodiments, the sustained remission is a clinical remission at about ten weeks from the first dose and at about 30 weeks from the first dose. In some embodiments, the sustained remission has a length of at least about 30 weeks, or at least about 7, about 8, about 9, about 10, about 11, or about 12 months. In some embodiments of any of the preceding aspects, the amelioration of one or more symptoms of the disease or condition, clinical remission, and/or clinical response is maintained at least one month (e.g., at least one month, at least two months, at least three months, at least four months, at least five months, at least six months, at least seven months, at least eight months, at least nine months, at least ten months, at least eleven months, at least twelve months, or longer) after the end of treatment.

### C. Exemplary Methods and Uses

In some embodiments, the provided methods and uses as described can be used in the treatment of an antibody-related disease or disorder, such as an autoantibody-related disease or disorder and/or B-cell-related disease. In some embodiments, the methods and uses are for treating an autoantibody-related disease or disorder. Any of such diseases or disorders as described, including any described in Section V.A, can be treated in accord with the provided methods and uses. In some embodiments, the methods and uses of the autoantibody-related disease or disorder, such as a B-cell related disease, can be by a dosing regimen as described in Section V.B or V.C. Exemplary methods and uses for administration of the provided pharmaceutical formulations containing a TACI-Fc fusion protein, as provided are described in the following subsections.

In some embodiments, the antibody-related disease or disorder is a rheumatic disease or disorder, a renal disease or disorder, a hematologic disease or disorder, a dermatologic disease or disorder, or a neurologic disease or disorder. The provided pharmaceutical formulations containing an immunomodulatory protein, including TACI-Fc fusion proteins as provided, can be used for the treatment of rheumatic disease, renal disease, hematologic disease, dermatologic disease or neurologic disease. In some embodiments, administration of the immunomodulatory proteins (e.g. TACI-Fc fusion protein) can specifically regulate B cell responses during the immune response. Additionally, administration of provided immunomodulatory proteins can be used to modulate B cell development, development of other cells, antibody production, and cytokine production. Administration or use of provided immunomodulatory proteins can also modulate B cell communication, such as by neutralizing the proliferative effects of BAFF or APRIL. In some embodiments, the provided immunomodulatory proteins can be used to block or neutralize the actions of B-cells in association with rheumatic disease, renal disease, hematologic disease, dermatologic disease or neurologic disease.

In some embodiments, administration or use of the provided immunomodulatory proteins (e.g., TACI Fc fusion) reduces the risk of the subject developing hypogammaglobulinemia. Hypogammaglobulinemia is a disorder caused by low serum immunoglobulin (e.g., IgG) or antibody levels, and encompasses a majority of immune-compromised patients. Huq et al., 2022 “Hypogammaglobulinemia.” StatPearls [Internet] StatPearls Publishing. Hypogammaglobulinemia can be of primary or secondary origin. Secondary causes are usually induced by an external or acquired factor such as a corticosteroid or immunosuppressant drug, nutritional disorders, infections, chemotherapy, malignancy, nephrotic syndrome, other metabolic diseases, and hazardous environmental conditions. Hypogammaglobulinemia can be determined according due to diagnostic criteria known in the art, which can be defined by local standard practice(s). For example, diagnostic criteria for hypogammaglobulinemia by the European Society of Immunodeficiency (ESID) require a substantial decrease in IgG concentration defined by at least two standard deviations below the average for healthy adults, which is 8 to 12 g/L (Huq et al., StatPearls Publishing, 2023). Thus, in some embodiments, hypogammalgobulinemia is diagnosed by immunoglobulin levels ≤7 g/L, ≤6 g/L, ≤5 g/L, ≤4 g/L, ≤3 g/L, 2 g/L, or ≤1 g/L. IgG is the most prominent circulating immunoglobulin in both the vascular and extravascular compartments, and thus, IgG is crucial to diagnosing hypogammaglobulinemia. Reduced IgA and IgM are also seen with low IgG levels. In specific embodiments, hypogammalgobulinemia is diagnosed by IgG levels ≤7 g/L, ≤6 g/L, ≤5 g/L, ≤4 g/L, ≤3 g/L, ≤2 g/L, or ≤1 g/L. In specific embodiments, hypogammalgobulinemia is diagnosed by IgG levels ≤3 g/L. In specific embodiments, hypogammalgobulinemia is diagnosed by IgG levels ≤1.5 g/L.

In some embodiments, minimizing the risk of the subject developing hypogammaglobulinemia means that the provided TACI Fc fusion proteins do not cause a reduction in IgG ≤7 g/L, ≤6 g/L, ≤5 g/L, ≤4 g/L, ≤3 g/L, ≤2 g/L, or ≤1 g/L in the subject. In some embodiments, minimizing the risk of the subject developing hypogammaglobulinemia means that the provided TACI Fc fusion proteins do not cause a reduction in ≤3 g/L. In some embodiments, minimizing the risk of the subject developing hypogammaglobulinemia means that the provided TACI Fc fusion proteins do not cause a reduction in ≤1.5 g/L.

In some embodiments, minimizing the risk of hypogammaglobulinemia occurs at the any of the doses or dose frequencies described in Section V.B. In some embodiments, the immunomodulatory protein provided herein (e.g., TACI fusion) reduces risk of hypogammaglobulinemia when administered at 80 mg SC Q4W. In some embodiments, the immunomodulatory protein provided herein (e.g., TACI fusion) reduces risk of hypogammaglobulinemia when administered at 240 mg SC Q4W.

### 1. Systemic Lupus Erythematosus (SLE)

In some embodiments, the provided TACI-Fc fusion proteins can be used for treating rheumatic diseases like autoantibody-mediated systemic lupus erythematosus (SLE).

SLE is a relapsing, remitting heterogeneous systemic autoimmune disease with mild to life-threatening manifestations. The most common symptoms of SLE include low-grade fever, photosensitivity, oral ulcers, muscle aches, arthritis, fatigue, loss of appetite, rash, pleuritis, pericarditis, nephritis and poor circulation.

of BAFF and APRIL increases under proinflammatory conditions (Smulski et al. 2018), and elevated serum levels of these cytokines have been correlated with disease severity in patients with B cell-related autoimmune disease, including systemic lupus erythematosus (SLE) (Samy et al. Int. Rev. Immunol. 2017 36:3-19). Binding of BAFF/APRIL to their receptors triggers events in B cell and plasma cell development, differentiation, and activation. For instance, activation of the BAFF-R contributes to survival and maturation of transitional and naïve B cells whereas TACI is involved in T cell-independent B cell responses to certain antigens, B cell regulation, and immunoglobulin (Ig) class-switch recombination. BCMA, which is upregulated in activated B cells, is important for the long-term survival of plasma cells.

In rheumatic diseases like SLE and other autoantibody-related disorders, current treatments include polypharmacy and fail to achieve remission in the majority of patients. Agents targeting the B-cell cytokines B-cell activating factor (BAFF) and/or a proliferation-inducing ligand (APRIL), including the monoclonal antibody belimumab and the wild-type (WT) transmembrane activator and calcium modulating cyclophilin ligand interactor (TACI)-Fc fusion proteins atacicept and telitacicept, have demonstrated promising clinical potential in such diseases. However, there still exists a large unmet need as current biologics require large doses or produce severe adverse effects such as hypertension, dyslipidemia, new onset diabetes, etc. (see, e.g., Belimumab, Anifrolumab, and Voclosporin). This is because BAFF and APRIL support B cell development, differentiation, and survival. Co-neutralization of BAFF and APRIL dramatically reduces B cell function, including antibody function. Thus, any disease where B cells play a substantial role in disease initiation and progression can be treated with the TACI-Fc fusion proteins provided herein.

The TACI-Fc fusion protein provided herein not only provides a well-tolerated and effective inhibitor of immune responses in models of SLE, but the TACI-Fc fusion protein provided herein is a potentially best-in-class therapy targeting clinically validated cytokines with applicability in other autoimmune diseases, like glomerulonephritis, cytopenia or blistering skin disease. Moreover, the TACI-Fc fusion protein provided herein provides a substantial improvement in patient convenience as dosing may be performed once every four weeks (Q4W) subcutaneously.

In particular embodiments, the provided formulations containing a TACI-Fc fusion protein are for treating SLE and other autoantibody-related rheumatic diseases for which there remain indications of high unmet need. In SLE, treatment options have been hindered by complex pathogenesis and heterogeneity of disease, suggesting that multiple pathways or aspects of B cell development and differentiation may require simultaneous inhibition to enable durable responses. While B cell-depleting agents such as rituximab/ocrelizumab/obinutuzumab (anti-CD20), and obexelimab (anti-CD19) have exhibited favorable clinical impacts in certain autoimmune disease settings, this has not translated to SLE, where rituximab failed to demonstrate benefit in SLE and LN trials (Merrill et al., Arthritis Rheum, 2010, 62(1):222-233; Rovin et al., Arthritis Rheum, 2012, 64(4):1215-1226). One possible limitation of these therapeutics is that CD20 and CD19 are not expressed on all ASC or LL-PC, and only earlier stage B cells (including pro/pre, immature, mature, and memory B cells) are depleted, sparing most pathogenic plasmablasts and PC (Lee et al., Nat Rev Drug Discov, 2021, 20(3):179-199; Arbitman et al., J Autoimmun, 2022, 102873).

In some aspects, provided herein is a method of treating Systemic lupus erythematosus (SLE) comprising administering the TACI-Fc fusion proteins provided herein. In some embodiments, the method comprises: a) selecting a subject for administration of a TACI-Fc fusion protein that has been diagnosed with SLE; and b) administering to the selected subject the TACI-Fc fusion protein in accord with provided methods and doses (e.g. described in Section V.B), wherein the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the Fc comprises the amino acid sequence set forth in SEQ ID NO:73. In some embodiments, the TACI-Fc fusion protein comprises the sequence of amino acids set forth in SEQ ID NO. 167. In some embodiments, the TACI-Fc fusion protein consists of the sequence of amino acids set forth in SEQ ID NO. 167. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section V.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg and is administered every four weeks (Q4W). In some embodiments, the dose is at or about 80 mg and is administered every four weeks (Q4W). In some embodiments, the dose is at or about 240 mg and is administered every four weeks (Q4W). In some embodiments, the dose is from at or about 80 mg to at or about 960 mg and is administered every four weeks (Q4W). In some embodiments, the dose is at or about 960 mg and is administered every four weeks (Q4W).

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, the SLE is mild to moderate SLE or moderate to severe SLE. In some embodiments, the subject is selected for treatment if at the time of screening the subject has active SLE for ≥6 months. In some embodiments, the subject having SLE is selected for treatment based on the level of protein in the urine as an indication of proteinuria. In some embodiments, the protein in the urine is urine total protein to creatinine ratio (UPCR; g/g). In some embodiments, the subject is selected for treatment if at the time of screening the SLE is characterized by one or more of the following: (i) a hybrid SELENA-SLEDAI score ≥8 or a hybrid SELENA-SLEDAI ≥6 if there is high anti-dsDNA or low complement (C) levels; (ii)≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0. In some embodiments, the SLE is mild to moderate. In some embodiments, the SLE is moderate to severe. In some embodiments, a subject has moderate to severe SLE if the subject has active SLE for ≥6 months. In some embodiments, a subject has moderate to severe SLE if the subject's SLE is characterized by one or more of the following: (i) a hybrid SELENA-SLEDAI score ≥8 or a hybrid SELENA-SLEDAI ≥6 if there is high anti-dsDNA or low complement (C) levels; (ii)≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0. In some embodiments, a subject with moderate to severe SLE is selected for treatment. In some embodiments, the subject having moderate to severe SLE is selected for treatment if the subject has active SLE for ≥6 months. In some embodiments, the subject having moderate to severe SLE is selected for treatment if the subject has SLE characterized by one or more of the following: (i) a hybrid SELENA-SLEDAI score ≥8 or a hybrid SELENA-SLEDAI ≥6 if there is high anti-dsDNA or low complement (C) levels; (ii)≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0.

In some embodiments, the subject is receiving standard therapy for treating the SLE. In some embodiments, the subject is selected for treatment if the at the time of screening or the time of administering the TACI-Fc fusion protein, the subject is receiving a stable standard treatment regimen characterized by the stable use of a standard therapy for treating the SLE. In some embodiments the stable use is stable use of the standard therapy for at least 30 days.

In some embodiments, the TACI-Fc fusion protein is administered to the subject in combination with a standard therapy for treating the SLE. In some embodiments, the standard therapy comprises one of more of a corticosteroid, antimalarial (e.g. hydroxychloroquine), a non-steroidal anti-inflammatory drug (NSAID), or an immunosuppressant or immunomodulator, or any combination thereof. In some embodiments, the immunosuppressant or immunomodulator is selected from the group consisting of including azathioprine, mycophenolate (e.g. mycophenolate mofetil or sodium mycophenolate), cyclophosphamide, methotrexate, leflunomide, tacrolimus, cyclosporine and combinations of any of the foregoing. In some embodiments, the mycophenolate comprises mycophenolic acid (MPA).

In some embodiments, the standard therapy comprises a corticosteroid and administration of the corticosteroid is tapered after administering the TACI-Fc fusion protein. In some embodiments, the subject is selected for treatment if at the time of screening the subject is characterized by one or more of the following: (i) severe lupus nephritis. In some embodiments severe lupus nephritis is such as defined as urine protein >6 g/24 hours or serum creatinine >2.5 mg/dL or 221 μmol/L; (ii) required hemodialysis; (iii) received high-dose corticosteroids for ≥14 days in the last 2 months, such as wherein the high-dose corticosteroid is treatment with prednisone >100 mg/day or equivalent; and (iv) central nervous system disease caused by SLE or not caused by SLE in the last 2 months. In some embodiments, the central nervous system disease is epilepsy, psychosis, organic brain syndrome, cerebrovascular accident, encephalitis, or central nervous system vasculitis.

In some embodiments, the autoantibody-related disease or disorder is a renal (kidney) disease or disorder. In some embodiments, the autoantibody-related disease or disorder is a Glomerulonephritis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

The TACI-Fc fusion protein may be provided in any of the formulations discussed herein. For example, in some embodiments, the TACI-Fc fusion protein is provided in a formulation comprising having a pH of from about 4.3 to about 5.1, about 5 mM to about 15 mM of an acetate, a tonicifying agent comprising arginine at or about 135 mM to 165 mM such as at or about 140 mM to 150 mM or an arginine-hydrochloride at a concentration of from at or about 1.5% to about 4% (w/v), and a non-ionic surfactant at a concentration of at or about 0.002% to about 1.0% (w/v), or at or about 0.01% to about 0.2% (w/v). In some cases, the surfactant is poloxamer 188 at or about 0.05% to about 0.2% (w/v) such as at or about 0.1% (w/v). In other embodiments, the surfactant is polysorbate 80 at or about 0.01% to about 0.04%. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the arginine hydrochloride is L-arginine hydrochloride. In some embodiments the formulation comprises arginine-hydrochloride at a concentration of about 3.1%. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is provided in a formulation comprising having a pH of from about 4.3 to about 5.2, about 5 mM to about 15 mM of an acetate, proline at a concentration of from at or about 1.5% to about 4%, a non-ionic surfactant at a concentration of from about 0.002% to about 1.0% (w/v), and a salt. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the proline is L-proline. In some embodiments the proline is at a concentration of about 1.5% to about 4%. In some embodiments the proline is at a concentration of at or about 2%. In some embodiments the proline is at a concentration of at or about 3%. In some embodiments, the surfactant is at a concentration of from about 0.05 to about 0.2% (w/v), such as at or about 0.1% (w/v). In some embodiments, the surfactant is poloxamer 188. In some embodiments, the surfactant is at a concentration of from about 0.01% to about 0.04%. In some embodiments, the surfactant is polysorbate 80. In some embodiments the salt is sodium chloride (NaCl). In some embodiments the amount of salt in the formulation is at or about 40 mM to at or about 100 mM. In some embodiments the amount of NaCl in the formulation is at or about 40 mM to at or about 100 mM. In some embodiments, the amount of salt, such as NaCl, is at or about 50 mM. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is administered intravenously. In some embodiments, the TACI-Fc fusion protein is administered subcutaneously.

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 2. Nephrology and Renal (Kidney) Disease

In some embodiments, the provided TACI-Fc fusion proteins can be used in the nephrology-based methods of treatment for treating autoantibody-mediated kidney disease. In some embodiments, the provided TACI-Fc fusion proteins can be used to treat an autoantibody-associated glomerular disease (also known as glomerulonephritis). In some embodiments, the autoantibody-associated glomerular disease may include immunoglobulin (Ig) A nephropathy (IgAN), lupus nephritis (LN), primary membranous nephropathy (pMN), or renal anti-neutrophil cytoplasmic antibody (ANCA)-associated vasculitis (AAV). In some embodiments, the autoantibody-associated glomerular disease may include focal segmental glomerulosclerosis (FSGS). In some embodiments, the autoantibody-associated glomerular disease may include minimal change disease (MCD).

Therapeutic agents targeting the B-cell cytokines BAFF and/or APRIL have demonstrated promising clinical potential in autoantibody-related GN such as LN and IgAN, and other B-cell-related diseases such as systemic lupus erythematosus; however, there is still need for more safe and efficacious therapies. TACI-Fc fusion protein provided herein mediates more potent inhibitory activity than WT TACI-Fc or BAFF- or APRIL-specific antibodies. In preclinical studies, the TACI-Fc fusion protein provided herein demonstrated enhanced PK and immunomodulatory properties vs. WT TACI-Fc, which may translate to lower and/or less frequent doses in human subjects. The TACI-Fc fusion protein provided herein also suppressed autoantibodies, renal IgG deposition, and nephritis in mouse models. Thus, the provided TACI-Fc fusion protein may significantly improve clinical outcomes in GN and other B-cell-related diseases.

APRIL plays a particularly important role in IgA class switching, production, and glycosylation, as first indicated by studies of APRIL knockout mice (Castigli et al., Proc Natl Acad Sci USA, 2004, 101(11):3903-3908). In addition, elevated plasma APRIL levels in IgA nephropathy (IgAN) patients are associated with more severe clinical manifestations such as high proteinuria and Gd (galactose deficient) IgA1 levels (Zhai et al., Medicine (Baltimore), 2016, 95(11):e3099), which are important causal factors and contribute to disease pathogenesis. Indeed, early trials of BION-1301 and sibeprenlimab suggest that APRIL-only inhibition can mediate significant decreases in Ig (particularly IgA) in healthy subjects, and BION-1301 impacts proteinuria in IgAN patients in an ongoing trial (Barratt et al., J Immunol, 2022, 180(6):3655-3659). However, targeting APRIL alone has its own limitations and would not be expected to impact less mature BAFF-dependent B cells that can also contribute to disease pathogenesis (Lee et al., Nat Rev Drug Discov, 2021, 20(3):179-199). BAFF neutralization leads to downregulation of B cell function, decreases in autoantibody production, and inhibition of tertiary lymphoid structure formation in the kidney (Samy, et al., Int Rev Immunol, 2017, 36(1):3-19).

In some embodiments, the provided. TACI-Fc can be used to treat immunoglobulin (Ig) A nephropathy (IgAN). In some embodiments, the IgAN diagnosis has been confirmed by biopsy within less than or equal to 3 years prior to screening or selection for administration, or initiation of administration, of the TACI-Fc fusion protein treatment. In some embodiments, a biopsy may be carried out on the subject prior to administration of the TACI-Fc fusion protein. In some embodiments, the subject is one that has elevated galactose deficient IgA1 (Gd-IgA1) antibodies at the time of, or when selected for, administration with the TACI-Fc fusion protein. In some embodiments, the TACI-Fc fusion protein provided herein may reduce inflammation and harmful antibodies that damage kidneys in subjects with IgAN. In some embodiments, the TACI-Fc fusion protein provided herein may reduce or reverse kidney damage associated with IgAN.

In some embodiments, the provided immunomodulatory proteins (e.g. TACI-Fc) can be used to treat lupus nephritis (LN). In some embodiments, the LN diagnosis has been confirmed by biopsy within less than or equal to 1 year prior to the initiation of screening for administration, or initiation of administration, of the TACI-Fc fusion protein treatment. In some embodiments, the subject is one that has a renal biopsy that shows evidence of active, proliferative Class III or IV LN per ISN/RPS criteria (see e.g. Markowitz and D'Agati, 2007, Kidney Int. 71:491-5). In some embodiments, the subject may co-exhibit Class V disease in addition to either Class III or Class IV disease. In some embodiments, a biopsy may be carried out on the subject prior to administration of the TACI-Fc fusion protein. In some embodiments, the subject has elevated anti-double stranded DNA (anti-dsDNA) at the time of, or when selected for, administration with the TACI-Fc fusion protein. In some embodiments, the subject is positive for anti-nuclear antibody (ANA) at the time of, or when selected for, administration with the TACI-Fc fusion protein. In some embodiments, the subject that is positive for ANA has a titer of greater than or equal to 1:80. In some embodiments, the subject that is positive for anti-dsDNA has a tier of greater than or equal to 30 IU/mL. In some embodiments, the TACI-Fc fusion protein provided herein may reduce inflammation and harmful antibodies that damage kidneys in subjects with LN. In some embodiments, the TACI-Fc fusion protein provided herein may reduce or reverse kidney damage associated with LN.

In some embodiments, the provided TACI-Fc can be used to treat primary membranous nephropathy (pMN). In some embodiments, the pMN diagnosis has been confirmed by biopsy within less than or equal to 3 years prior to screening or selection for administration, or initiation of administration, of the TACI-Fc fusion protein treatment. In some embodiments, a biopsy may be carried out on the subject prior to administration of the TACI-Fc fusion protein. In some embodiments, the subject is positive for anti-phospholipase A2 receptor 1 (anti-PLA2R1) antibodies and/or anti-thrombospondin type-1 domain-containing 7A (anti-THSD7A) antibodies at the time of, or when selected for, administration with the TACI-Fc fusion protein. In some embodiments, the subject having SLE is selected for treatment based on the level of protein in the urine as an indication of proteinuria. In some embodiments, the protein in the urine is urine total protein to creatinine ratio (UPCR; g/g). In some embodiments, the subject is one that has less than 50% reduction of proteinuria in the last 24 weeks while on angiotensin-converting enzyme (ACE)/angiotensin receptor blockade (ARB). In some embodiments, the subject has received maximal ACE/ARB therapy for ≥12 weeks prior to screening or selection for administration, or initiation of administration, with stable blood pressure (BP) therapy. In some embodiments, a stable blood pressure comprises a normal blood pressure. In some embodiments, a normal blood pressure comprises less than or equal to 120/80 mmHg. In some embodiments, a normal blood pressure comprises less than or equal to 140/70 mmHg. In some embodiments, the subject is not receiving any concomitant medications that are considered prohibited in connection with administration of a TACI-Fc fusion protein, such as determined by a clinician or physician. In some embodiments, the TACI-Fc fusion protein provided herein may reduce inflammation and harmful antibodies that damage kidneys in subjects with pMN. In some embodiments, the TACI-Fc fusion protein provided herein may reduce or reverse kidney damage associated with pMN.

In some embodiments, the subject to be selected or treated is one that has not had a prior diagnosis of, or fulfills diagnostic criteria for, another glomerular disease, has an eGFR <30 mL/min/1.73 m2 or rapidly progressive glomerulonephritis, has recent serious or ongoing infection, and/or risk or history of serious infection.

In some embodiments, the provided immunomodulatory proteins (e.g. TACI-Fc) can be used to treat renal anti-neutrophil cytoplasmic antibody (ANCA)-associated vasculitis (AAV). In some embodiments, the renal AAV diagnosis has been confirmed by biopsy within less than or equal to 23 years prior to screening or selection for administration, or initiation of administration, of the TACI-Fc fusion protein treatment. In some embodiments, the biopsy confirms evidence of renal ANCA-associated vasculitis. In some embodiments, a biopsy may be carried out on the subject prior to administration of the TACI-Fc fusion protein. In some embodiments the subject is positive for anti-proteinase 3 (PR3) or anti-myeloperoxidase (MPO) antibodies at the time of, or when selected for administration with the TACI-Fc fusion protein.

In some embodiments, the subject may receive standard of care (SOC) therapy for their underlying disorder, which is within the level of skill of the investigator or clinical physician. In some embodiments, the SOC therapy may include a renin-angiotensin-aldosterone system inhibitor (RAASi), a statin, a diuretic, an immune modulator, an immunosuppressant or a corticosteroid. In some embodiments, the subject has previously received the SOC therapy prior to receiving the provided TACI-Fc fusion protein. In some embodiments, the subject continues receiving the SOC therapy while receiving administration of the provided TACI-Fc fusion protein. In some embodiments, the SOC therapy is tapered over time after receiving administration of the provided TACI-Fc fusion protein. In some embodiments, the SOC therapy may include an antimalarial, an antibiotic such as a tetracycline, a steroid such as prednisone, a sodium-glucose cotransporter-2 (SGLT2) inhibitors, mycophenolate mofetil (MMF), mycophenolic acid (MPA), voclosporin or other SOC therapy within the level of a skilled artisan. In some embodiments, just prior to the initiation of administration of the TACI-Fc the subject has not received, or is not receiving, combination therapy with two immunomodulatory treatments, such as MMF and voclosporin.

In some embodiments, the subject has LN or renal AAV and the subject has received therapy with mycophenolate mofetil (MMF)/mycophenolic acid (MPA) or other immunotherapy as a standard of care therapy for treating the LN or renal AAV. In some embodiments, the subject is administered mycophenolate mofetil (MMF)/mycophenolic acid (MPA) or other immunotherapy as a standard of care therapy for treating the LN or renal AAV, prior to or during the administration of the provided TACI-Fc fusion protein. In some embodiments, the subject has not received a steroid within 5 days prior to the initiation of administration of the provided TACI-Fc fusion protein.

In some embodiments, the subject that is administered a provided immunomodulatory proteins (e.g. TACI-Fc) in accord with the provided methods does not have another renal disease including but not limited to diabetic nephropathy; C3 glomerulonephropathy; focal segmental glomerulosclerosis; thin basement membrane disease; Alport's disease; IgA vasculitis; minimal change disease; post-infectious glomerulonephritis; secondary membranous nephropathy (excluding LN Class V combined with Class III or IV); or secondary IgAN including but not limited to Celiac disease, Crohn's disease, HIV, or liver cirrhosis.

In some embodiments, the subject has not received an agent that directly depletes B lymphocytes (e.g. Rituximab) within 48 weeks prior to initiation of administration of the provided immunomodulatory proteins (e.g. TACI-Fc). In some embodiments, the subject may have received an agent that directly depletes B lymphocytes (e.g. Rituximab) within greater than 24 weeks prior to initiation of administration of the provided immunomodulatory proteins (e.g. TACI-Fc) if B cells have returned to normal reference ranges prior to administration of the TACI-Fc fusion protein.

In some embodiments, the subject has not received an agent that directly inhibits BAFF and/or APRIL, such as Belimumab, within 24 weeks prior to initiation of administration of the provided immunomodulatory proteins (e.g. TACI-Fc).

In some embodiments, the subject has not received administration of Intravenous Ig, abatacept, anifrolumab, belatacept, adalimumab, infliximab, certolizumab, etanercept, golimumab, anakinra, canakinumab, tocilizumab, sarilumab, satralizumab within 8 weeks prior to initiation of administration of the provided immunomodulatory proteins (e.g. TACI-Fc). In some embodiments, the subject has not received administration of any approved therapeutic agent for treating an immune disease within 8 weeks prior to initiation of administration of the provided immunomodulatory proteins (e.g. TACI-Fc).

In some embodiments, the subject has not received cyclophosphamide within 8 weeks prior to initiation of administration of the provided immunomodulatory proteins (e.g. TACI-Fc).

In some embodiments, the efficacy of the treatment is monitored in the subject. In some embodiments, the change in baseline over time in circulating levels of antibodies, such as autoantibodies are monitored in the subject.

In some embodiments, the subject has LN and a change from baseline over time of circulating levels of anti-dsDNA is monitored in the subject.

In some embodiments, the subject has IgAN and a change from baseline over time of circulating levels of Gd-IgA1 and anti-Gd-IgA1 is monitored in the subject. In some embodiments, the subject will have greater than a 50%, greater than a 55%, greater than a 60%, greater than a 65%, greater than a 70%, greater than a 75%, greater than an 80%, greater than a 85%, greater than a 90%, or greater than a 95% reduction in Gd-IgA1. In some embodiments, the subject will have greater than a 50% reduction in Gd-IgA1. In other embodiments, greater than 75%, greater than 80%, greater than 85%, greater than 90%, or greater than 95% of subjects receiving the TACI-Fc fusion protein will have a reduction in Gd-IgA1. In specific embodiments, greater than 75% of subjects receiving the TACI-Fc fusion protein will have greater than a 50% reduction in Gd-IgA1.

In some embodiments, the subject has pMN and a change from baseline over time of circulating levels of pMN and anti-MPO is monitored in the subject.

In some embodiments, the subject has renal AAV and a change from baseline over time of circulating levels of anti-PR3 is monitored in the subject.

In some embodiments, a change in baseline over time of a complement component is monitored in the subject. In some embodiments, the complement component is one or more of C3, C4 or CH50.

In some embodiments, a clinical response is monitored in the subject. In some embodiments, the clinical response may be assessed by monitoring baseline estimated glomerular filtration rate (eGFR) over time. In some embodiments, eGFR is calculated by an equation that uses serum creatine or cystatin C. In some embodiments, the eGFR is calculated by an equation that is independent of race, such as described in Inker et al., 2021 N Engl J Med., 385:1737-1749. In some embodiments, the eGFR may be estimated using the Chronic Kidney Disease Epidemiology Collaboration (CKD-EPI) formula.

In some embodiments, the response is measured as a renal response by determination of eGFR (e.g. using cystatin C race-independent equation). In some embodiments, the renal response is measured in subjects with LN or pMN.

In some embodiments, the subject has LN and complete renal response is present if the subject has UPCR less than 0.5 g/g (e.g. based on 24-hour urine collection) and eGFR is greater than or equal to the lower limit of normal (LLN) or there has been a less than 20% decrease in eGFR from baseline where the eGFR is less than LLN. In some embodiments, the subject has LN and a partial renal response is present if the subject has UPCR less than or equal to 3.5 g/g and a greater than 50% reduction from baseline (e.g. based on 24-hour urine collection), and eGFR is greater than or equal to 60 mL/min/1.73 m² or there is a less than a 20% decrease of eGFR from baseline.

In some embodiments, the subject has pMN and complete renal response is present if the subject has UPCR less than 0.3 g/g (e.g. based on 24-hour urine collection), serum albumin greater than 35 g/L, and eGFR is greater than or equal to 60 mL/min/1.73 m². In some embodiments, the subject has pMN and a partial renal response is present if the subject has UPCR less than or equal to 3.5 g/g and a greater than 50% reduction from baseline (e.g. based on 24-hour urine collection), serum albumin greater than 30 g/L, and stable eGFR (e.g. decline of less than 15% compared to baseline.

In some aspects, provided herein is a method of treating glomerulonephritis comprising administering the TACI-Fc fusion proteins in a formulation provided herein. In some embodiments, the method comprises a) selecting a subject for administration of a TACI-Fc fusion protein that has been diagnosed with glomerulonephritis; and b) administering to the selected subject the TACI-Fc fusion protein in accord with provided methods and doses (e.g. as described in Section V.B), wherein the formulation comprises a TACI-Fc fusion protein that is a homodimer of two polypeptides of the formula TACI-linker-Fc, wherein TACI polypeptide is a wild type TACI polypeptide or variant TACI polypeptide such as any provided herein. In some embodiments, the variant TACI polypeptide comprises the amino acid substitutions K77E, F78Y and Y102D in the amino acid sequence set forth in SEQ ID NO:13. In some embodiments, the Fc comprises the amino acid sequence set forth in SEQ ID NO:73. In some embodiments, the TACI-Fc fusion protein comprises the sequence of amino acids set forth in SEQ ID NO. 167. In some embodiments, the TACI-Fc fusion protein consists of the sequence of amino acids set forth in SEQ ID NO. 167. In some embodiments, TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section V.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the subject is selected for treatment if at the time of screening the subject has active Glomerulonephritis.

In some embodiments, the Glomerulonephritis is selected from the group consisting of IgA Nephropathy, Lupus Nephritis and Primary Membranous Nephropathy. In some embodiments, the subject is greater than or equal to (≥) 18 years of age. In some embodiments, the subject having IgAN, LN or pMN is selected for treatment based on the level of protein in the urine as an indication of proteinuria. In some embodiments, the protein in the urine is urine total protein to creatinine ratio (UPCR; g/g). In some embodiments, the subject has received a proteinuria-lowering medication, such as combination of an ACE inhibitor (ACEI) and an ARB. In some embodiments, the proteinuria-lowering medication is titrated to the maximal dose that can be tolerated without adversely effecting systemic blood pressure GFR or serum potassium levels. In some embodiments, the stable maximal dose of a proteinuria-lowering medication, such as ACEI/ARB, has been administered for ≥12 weeks. In some embodiments, the subject has received stable background immunosuppression prior to treatment with the TACI-Fc. In some embodiments, the subject intends to maintain stable background immunosuppression through the treatment period with the TACI-Fc fusion protein. In some embodiments, the subject has not received any background immunosuppression (e.g. MMF ≥1 g/day with or without corticosteroids) prior to treatment with the TACI-Fc, but optionally may receive stable calcineurin inhibitors (CnI).

In some embodiments, the Glomerulonephritis is IgA Nephropathy (IgAN) and is characterized by urine total protein to creatinine ratio (UPCR) greater than or equal to 0.5 g/g, such as greater than 0.6 g/g, 0.65 g/g, 0.70 g/g or 0.75 g/g UPCR (proteinuria). In some embodiments, the subject has received standard care of therapy, such as stable background immunosuppression. In some embodiments, the stable immunosuppression includes one or more of MMF (e.g. a stable dose of MMF of ≥1 g/day), corticosteroids, azathioprine (AZA), or calcineurin inhibitors (CnI). In some embodiments, the subject has not received standard of care therapy, such as has not received stable background immunosuppression.

In some embodiments, the subject having Glomerulonephritis, such as IgAN, LN or pMN, is not selected for treatment if at the time of screening the subject is characterized by one or more of the following: (i) eGFR <30 mL/min/1.73m2; (ii) rapidly progressive glomerulonephritis; (iii) sclerosis or interstitial fibrosis and tubular atrophy in >50% of the biopsy; (iv) receiving prior B cell depletion or BAFF/APRIL inhibitors for ≤24 weeks; (v) history of demyelinating disorder; and (vi) known immunodeficiency, including IgG <700 mg/dL, IgA <10 mg/dL, B cells <100 per μL, or CD4<200 per mm³.

In some embodiments, the Glomerulonephritis is IgA Nephropathy (IgAN) and the subject is selected for treatment if at the time of screening the subject is characterized by one or both of the following: (i) the subject was diagnosed (e.g., by biopsy) with IgA Nephropathy (IgAN) ≤5 years prior to the screening; and (ii) the subject has ≥0.75 g/g urine total protein-creatine ratio (proteinuria).

In some embodiments, the Glomerulonephritis is IgAN and the subject is selected for treatment if at the time of screening the subject is characterized by one or both of the following: (i) the subject has been diagnosed (e.g., by biopsy) with IgA Nephropathy (IgAN) ≤5 years prior to the screening; and (ii) the subject has ≥0.5 g/g urine total protein-creatine ratio (proteinuria); and the subject has not received stable background immunosuppression.

In some embodiments, the Glomerulonephritis is Lupus Nephritis and the Lupus Nephritis is Class III (active focal), Class IV (diffuse) or Class V (lupus membranous nephropathy). In some embodiments, the Lupus Nephritis is Class III-V. In some embodiments, the Lupus Nephritis is characterized by proteinuria greater than or equal to ≥1.0 g/g urine total protein to creatinine ratio (UPCR), such as UPCR ≥2.0 g/g, UPCR ≥2.5 g/g, UPCR ≥3.0 g/g, UPCR ≥3.5 g/g. In some embodiments, the subject is characterized by UPCR ≥1.0 g/g with active urinary sediment. In some embodiments, the subject is characterized by UPCR ≥3.5 g/g. In some embodiments, the subject has received standard care of therapy, such as stable background immunosuppression. In some embodiments, the subject has not received stable background immunosuppression (e.g. MMF ≥1 g/day).

In some embodiments, the Glomerulonephritis is Lupus Nephritis (LN) and the subject is selected for treatment if at the time of screening the subject is characterized by one or of the following: (i) the subject was diagnosed (e.g. biopsy) with Lupus Nephritis Class II-V ≤3 years prior to the screening; (ii) UPCR ≥1 g/g urine total protein-creatinine ratio (proteinuria); (iii) active urinary sediment; (iv) positive anti-dsDNA and antinuclear antibodies (ANA), such as wherein positive anti-dsDNA is a titer of ≥30 IU/mL and positive ANA is a titer of ≥1:80; and (v) received stable background immunosuppression, such as wherein the stable background immunosuppression is a stable dose of MMF of ≥1 g/day, with or without corticosteroids, for at least 8 weeks prior to the time of screening or the time of administering the TACI-Fc fusion protein. In some embodiments, the Glomerulonephritis is Lupus Nephritis (LN) and the subject is selected for treatment if at the time of screening the subject is characterized by
(i) the subject was diagnosed (e.g., by biopsy) with Lupus Nephritis Class III-V ≤3 years prior to the screening;
(ii) UPCR ≥1 g/g with active urinary sediment; biopsy ≤24 weeks; or UPCR ≥3.5 g/g; (iii) positive anti-dsDNA and antinuclear antibodies (ANA), such as wherein positive anti-dsDNA is a titer of ≥30 IU/mL and positive ANA is a titer of ≥1:80;
(iv) received stable background immunosuppression, such as wherein the stable background immunosuppression is a stable dose of MMF of ≥1 g/day, with or without corticosteroids; and (v) is on a maintenance treatment such as with of MMF (e.g., of ≥1 g/day), azathioprine (AZA), or calcineurin inhibitors (CnI).

In some embodiments, the Glomerulonephritis is primary Membranous Nephropathy. In some embodiments, the Glomerulonephritis is primary Membranous Nephropathy (pMN) and the subject is selected for treatment if at the time of screening the subject is characterized by one or both of the following: (i) the subject was diagnosed with pMN ≤5 years prior to the screening; and (ii) ≥3.5 g/g urine total protein to creatinine ratio (proteinuria); and (iii) positive anti-PLA2R1 or positive anti-THSD7A antibodies.

In some embodiments, the Glomerulonephritis is pMN and the subject is selected for treatment if at the time of screening the subject is characterized by one or more of the following:
(i) the subject was diagnosed (e.g. biopsy-confirmed) with pMN ≤5 years prior to the screening;
(ii) the subject has UPCR ≥3.5 g/g;
(iii) positive anti-PLA2R1 or positive anti-THSD7A antibodies;
(iv) less than 50% reduction of proteinuria in the past 24 weeks while on stable maximal dose of angiotensin-converting enzyme inhibitor (ACEi)/angiotensin receptor blocker (ARB); and (v) has not received stable background immunosuppression, except optionally may receive a stable dose of calcineurin inhibitor (CnI) for ≥12 weeks.

In some embodiments, the subject is selected for treatment if at the time of screening or the time of administering the TACI-Fc fusion protein the subject has received therapy with an Angiotensin-converting enzyme (ACE) inhibitor and/or angiotensin II receptor blocker (ARB), such as wherein the subject has received a maximally recommended dose of the ACE inhibitor or ARB therapy. In some embodiments, the subject is selected for treatment if at the time of screening or the time of administering the TACI-Fc fusion protein the subject has received therapy with an SGLT2 inhibitor. In some embodiments, the subject is selected for treatment if at the time of screening or the time of administering the TACI-Fc fusion protein the subject has received therapy with an endothelin receptor antagonist. In some embodiments, the subject is selected for treatment if at the time of screening or at the time of administering the TAC-Fc fusion protein the subject has a stable blood pressure.

In some embodiments, the autoantibody-related disease or disorder is a hematological disease or disorder. In some embodiments, the autoantibody-related disease or disorder is an autoimmune cytopenia.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

The TACI-Fc fusion protein may be provided in any of the formulations discussed herein. For example, in some embodiments, the TACI-Fc fusion protein is provided in a formulation comprising having a pH of from about 4.3 to about 5.1, about 5 mM to about 15 mM of an acetate, a tonicifying agent comprising arginine at or about 135 mM to 165 mM such as at or about 140 mM to 150 mM or an arginine-hydrochloride at a concentration of from at or about 1.5% to about 4% (w/v), and a non-ionic surfactant at a concentration of at or about 0.002% to about 1.0% (w/v), or at or about 0.01% to about 0.2% (w/v). In some cases, the surfactant is poloxamer 188 at or about 0.05% to about 0.2% (w/v) such as at or about 0.1% (w/v). In other embodiments, the surfactant is polysorbate 80 at or about 0.01% to about 0.04%. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the arginine hydrochloride is L-arginine hydrochloride. In some embodiments the formulation comprises arginine-hydrochloride at a concentration of about 3.1%. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is provided in a formulation comprising having a pH of from about 4.3 to about 5.2, about 5 mM to about 15 mM of an acetate, proline at a concentration of from at or about 1.5% to about 4%, a non-ionic surfactant at a concentration of from about 0.002% to about 1.0% (w/v), and a salt. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the proline is L-proline. In some embodiments the proline is at a concentration of about 1.5% to about 4%. In some embodiments the proline is at a concentration of at or about 2%. In some embodiments the proline is at a concentration of at or about 3%. In some embodiments, the surfactant is at a concentration of from about 0.05 to about 0.2% (w/v), such as at or about 0.1% (w/v). In some embodiments, the surfactant is poloxamer 188. In some embodiments, the surfactant is at a concentration of from about 0.01% to about 0.04%. In some embodiments, the surfactant is polysorbate 80. In some embodiments the salt is sodium chloride (NaCl). In some embodiments the amount of salt in the formulation is at or about 40 mM to at or about 100 mM. In some embodiments the amount of NaCl in the formulation is at or about 40 mM to at or about 100 mM. In some embodiments, the amount of salt, such as NaCl, is at or about 50 mM. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is administered intravenously. In some embodiments, the TACI-Fc fusion protein is administered subcutaneously.

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, during the course of the treatment, the subject may be monitored for one or more endpoints of the disease to assess efficacy and/or one or more for an adverse event. In some embodiments, efficacy is monitored by an improvement in UPCR. In some embodiments, UPCR improves by at least or about 10% to 30%, 20% to 40%, 30% to 50%, 40% to 60%, 50% to 70%, 60% to 80%, 70% to 90%, or 80% to 100%. In some embodiments, the UPCR improves by at least or about 15%, 20%, 25%, 30%, or 35% compared to baseline. In some embodiments, the UPCR improves by at least or about 40%, 50%, 60%, 70% or 80% compared to baseline. In some embodiments, the UPCR improves by at least or about 25% compared to baseline. In some embodiments, the UPCR improves by at least or about 30%. In some embodiments, the UPCR improves by at least or about 40%. In some embodiments, the UPCR improves by at least or about 50%. In some embodiments, the UPCR improves by at least or about 70%. In some embodiments, the UPCR improves by at least or about 80%. In some embodiments, the UPCR improves by at least or about 100%.

In some embodiments, during the course of the treatment, the subject may be monitored for one or more endpoints of the disease to assess efficacy and/or one or more for an adverse event. In some embodiments, efficacy is monitored by a reduction in UPCR. In some embodiments, UPCR is reduced by at least or about 10% to 30%, 20% to 40%, 30% to 50%, 40% to 60%, 50% to 70%, 60% to 80%, 70% to 90%, or 80% to 100%. In some embodiments, the UPCR is reduced by at least or about 15%, 20%, 25%, 30%, or 35% compared to baseline. In some embodiments, the UPCR is reduced by at least or about 40%, 50%, 60%, 70% or 80% compared to baseline. In some embodiments, the UPCR is reduced by at least or about 25% compared to baseline. In some embodiments, the UPCR is reduced by at least or about 30%. In some embodiments, the UPCR is reduced by at least or about 40%. In some embodiments, the UPCR is reduced by at least or about 50%. In some embodiments, the UPCR is reduced by at least or about 70%. In some embodiments, the UPCR is reduced by at least or about 80%. In some embodiments, the UPCR reduced by at least or about 100%.

In some embodiments, the subject achieves complete remission as defined by a UPCR of <0.5 mg/mg.

In some embodiments, efficacy is monitored by a reduction in serum Ig levels. In some embodiments, serum Ig levels include, but are not limited to, serum IgA, serum IgG, and/or serum IgM levels. In some embodiments, serum Ig levels are reduced by at least or about 10% to 30%, 20% to 40%, 30% to 50%, 40% to 60%, 50% to 70%, 60% to 80%, 70% to 90%, or 80% to 100%. In some embodiments, serum Ig levels are reduced by at least or about 1%, 5%, 10%, 15%, 20%, 25%, 30%, 35%, 40%, 45%, 50%, 55%, 60%, 65%, 70%, 75%, 80%, 85%, or 90% compared to baseline. In some embodiments, serum Ig levels are reduced by at least or about 10%, 20%, 30%, 40%, 50%, 60%, 70%, 80%, or 90% compared to baseline. In some embodiments, serum Ig levels are reduced by at least or about 40%.

In some embodiments, the IgA comprises Gd-IgA1. In some embodiments, serum Gd-IgA1 levels are reduced by at least or about 10% to 30%, 20% to 40%, 30% to 50%, 40% to 60%, 50% to 70%, 60% to 80%, 70% to 90%, or 80% to 100%. In some embodiments, serum Gd-IgA1 levels are reduced by at least or about 1%, 5%, 10%, 15%, 20%, 25%, 30%, 35%, 40%, 45%, 50%, 55%, 60%, 65%, 70%, 75%, 80%, 85%, or 90% compared to baseline. In some embodiments, serum Gd-IgA1 levels are reduced by at least or about 10%, 20%, 30%, 40%, 50%, 60%, 70%, 80%, or 90%.

In some embodiments, efficacy is monitored by a reduction in serum anti-phospholipase A2 receptor (PLA2R). In some embodiments, serum anti-PLA2R levels are reduced by at least or about 1%, 5% 10%, 15%, 20%, 25%, 30%, 35%, 40%, 45%, 50%, 55%, 60%, 65%, 70%, 75%, 80%, 85%, or 90% compared to baseline. In some embodiments, serum anti-PLA2R levels are reduced by at least or about 10%, 20%, 30%, 40%, 50%, 60%, 70%, 80%, or 90% compared to baseline. In some embodiments, serum anti-PLA2R levels are reduced by at least or about 30%. In some embodiments, serum anti-PLA2R levels are reduced by at least or about 40%.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 3. Hematologic Disease

In some embodiments, the provided TACI-Fc fusion proteins can be used for treating hematologic diseases like autoimmune cytopenia.

In preclinical studies, co-neutralization of BAFF and APRIL can directly suppress antibody-secreting cells (ASC) and reduce circulating immunoglobulins. Autoimmune hemolytic anemia (AIHA) and immune thrombocytopenic purpura (ITP) are cytopenia diseases characterized by autoantibodies directed against red blood cells and platelets, respectively. Significantly higher levels of both BAFF and APRIL have been observed in the serum of patients with AIHA and ITP compared to healthy subjects, and polymorphisms in BAFF and TACI have been associated with ITP (Abdel-Hamid et al. (2011) Am J Med; Emmerich et al. (2007) Br J Haematol; Peng et al. (2017) J Thromb Haemost; Yu et al. (2021) Blood Adv). Currently, there are no B cell targeting agents approved to treat AIHA or ITP. Rituximab, an anti-CD20 therapeutic that depletes CD20+ B cells and thereby only indirectly reduces autoantibody production, has demonstrated some efficacy in both ITP (Neunert et al. (2019) Blood) and AIHA (Jager et al. (2019) Blood Rev), though it is not approved for use in either indication. Belimumab, an anti-BAFF neutralizing antibody (Ab), has demonstrated encouraging efficacy in the treatment of ITP associated with systemic lupus erythematosus (SLE) (De Marchi et al. (2017) Clin Exp Rheumatol) and also in combination with rituximab in patients with ITP (Mahévas et al. (2021) Haematologica), but only had a modest impact on hematological manifestations in SLE patients (Manzi et al. (2012) Ann Rheum Dis.

The provided TACI-Fc fusion protein can address the shortcomings of existing B-cell-targeting agents, by substantially improving upon the ligand affinity liabilities of wild type (WT) TACI, resulting in highly potent dual BAFF/APRIL inhibition superior to WT TACI-Fc, or to BAFF- or APRIL-specific monoclonal Abs. In preclinical studies, the TACI-Fc fusion protein provided herein demonstrates enhanced pharmacokinetic (PK) and immunomodulatory properties vs. WT TACI-Fc, which may translate to lower and/or less frequent doses in human subjects. The provided TACI-Fc fusion protein also suppresses autoantibodies and nephritis in mouse models of lupus. Taken together, these observations suggest that a potent dual BAFF/APRIL antagonist, such as the TACI-fusion protein provided herein, can lower pathogenic autoantibody levels, and may be particularly beneficial in the treatment of antibody-related hematologic diseases, such as autoimmune cytopenias.

In some aspects, provided herein is a method of treating autoimmune cytopenia comprising administering the TACI-Fc fusion proteins in a formulation provided herein. In some embodiments, the method comprises a) selecting a subject for administration of a TACI-Fc fusion protein that has been diagnosed with an autoimmune cytopenia; and b) administering to the selected subject the TACI-Fc fusion protein in accord with the provided methods and doses (e.g. as described in Section V.B), wherein the formulation comprises a TACI-Fc fusion protein that is a homodimer of two polypeptides of the formula TACI-linker-Fc, wherein TACI polypeptide is a wild-type TACI polypeptide or is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the Fc comprises the amino acid sequence set forth in SEQ ID NO:73. In some embodiments, the TACI-Fc fusion protein comprises the sequence of amino acids set forth in SEQ ID NO. 167. In some embodiments, the TACI-Fc fusion protein consists of the sequence of amino acids set forth in SEQ ID NO. 167. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section V.B. In some embodiments, the dose is from at or about 80 mg to at or about 240 mg. In some embodiments, the dose is at or about 80 mg. In some embodiments, the dose is at or about 240 mg. In some embodiments, the dose is from at or about 80 mg to at or about 960 mg Q4W. In some embodiments, the dose is at or about 960 mg Q4W.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W.

In some embodiments, the subject is selected for treatment if at the time of screening the subject has active cytopenia. In some embodiments, the autoimmune cytopenia is selected from the group consisting of Immune Thrombocytopenia (ITP) and Autoimmune Hemolytic Anemia (AIHA). In some embodiments, the subject is greater than or equal to 18 years of age. In some embodiments, the subject intends to maintain any background therapy through the treatment period. In some embodiments, the background therapy comprises corticosteroids (e.g., at a dose ≤20 mg), thrombopoietin receptor agonists (TPO-RA), erythropoiesis-stimulating agents (ESA), or oral immunosuppressants. In some embodiments, the cytopenia is ITP and is characterized by urine total protein to creatinine ratio (UPCR) greater than or equal to 2 g/g, such as greater than 2.5 g/g/, 3 g/g, 3.5 g/g or 4 g/g urine total protein to creatinine ratio (UPCR).

In some embodiments, the subject having autoimmune cytopenia, such as ITP, wAIHA or CAD, is not selected for treatment if at the time of screening the subject is characterized by one or more of the following: (i) secondary autoimmune cytopenia including Evans Syndrome; (ii) received prior B cell depletion (BCD) or BAFF/APRIL inhibitors for <12 weeks, however, BCD for 12 weeks is ok if B cells and Ig levels are normal; (iii) history of demyelinating disorder; and (iv) known immunodeficiency, including IgG <700 mg/DL, IgA <10 mg/dL, B cells <100 per μL, or CD4 <200 per mm³.

In some embodiments, the autoimmune cytopenia is ITP and the subject is selected for treatment if at the time of screening the subject is characterized by one or more of the following:
(i) the subject was diagnosed with ITP ≥3 months prior to the screening;
(ii) sustained low platelet counts (e.g., <30,000/μL); and
(iii) received ≥4 prior treatments for treating the ITP.

In some embodiments, the autoimmune cytopenia is ITP and the subject is selected for treatment if at the time of screening the subject is characterized by one or more of the following:
(i) the subject was diagnosed with ITP ≥3 months prior to the screening;
(ii) sustained low platelet counts (e.g., <30,000/μL);
(iii) received ≥2 prior treatments for treating the ITP, including ≥1 TPO-RA and ≥prior response; and
(iv) no concomitant immunosuppression, except for corticosteroids (e.g., ≤20 mg).

In some embodiments, the TACI-Fc fusion protein provided herein may reduce harmful antibodies that attack and destroy platelets in subjects with ITP.

In some embodiments, the TACI-Fc fusion protein provided herein may reduce harmful antibodies that attack and destroy red blood cells in subjects with ITP.

In some embodiments, the autoimmune cytopenia is an AIHA and the AIHA is warm AIHA (wAIHA) or cold AIHA (cAIHA or cold agglutinin disease, CAD). In some embodiments, the cytopenia is wAIHA or CAD and is characterized by sustained hemoglobin greater than 9 g/dL such as greater than 9.5 g/dL or 10 g/dL.

In some embodiments, the autoimmune cytopenia is wAIHA or CAD and the subject is selected for treatment if at the time of screening the subject is characterized by one or more of the following:
(i) the subject was diagnosed with wAIHA or CAD ≥3 months prior to the screening;
(ii) sustained hemoglobin (Hb) <9 g/dL;
(iii) received ≥2 prior treatments for treating the wAIHA or CAD; and
(iv) is experiencing symptoms from anemia.

In some embodiments, the autoimmune cytopenia is wAIHA or CAD and the subject is selected for treatment if at the time of screening the subject is characterized by one or more of the following:
(i) the subject was diagnosed with primary disease (wAIHA or CAD) ≥12 weeks prior to the screening and ≥2 prior treatment regimens;
(ii) sustained hemoglobin (Hb) <10 g/dL;
(iii) received ≥2 prior treatments for treating the wAIHA or CAD;
(iv) is experiencing symptoms from anemia;
(v) IgG+ direct antiglobulin (DAT) test or cold agglutinin (CAD) titer; and
(vi) evidence of hemolysis by haptoglobin, iBili, or LDH.

In some embodiments, the autoimmune cytopenia is warm autoimmune hemolytic anemia (wAIHA).

In some embodiments, the autoimmune cytopenia is cold autoimmune hemolytic anemia (cAIHA or CAD).

In some embodiments, the TACI-Fc fusion protein provided herein may reduce harmful antibodies that attack and destroy platelets in subjects with CAD.

In some embodiments, the subject is selected for treatment if at the time of screening or the time of administering the TACI-Fc fusion protein, the subject is receiving a stable immunosuppression. In some embodiments, the TACI-Fc fusion protein is administered to the subject in combination with concurrent administration of the stable immunosuppression. In some embodiments, the stable immunosuppression comprises a stable dose of a steroid, such as a corticosteroid, for at least two weeks prior to the time of screening or the time of administering the TACI-Fc fusion protein; and/or the stable immunosuppression comprises a stable dose of azathioprine, MMF, or cyclosporine for at least four weeks prior to the time of screening or the time of administering the TACI-Fc fusion protein. In some embodiments, the stable immunosuppression comprises a calcineurin inhibitor (e.g., cyclosporine or tacrolimus) for at least four weeks prior to the time of screening or the time of administering the TACI-Fc fusion protein.

In some embodiments, the subject is not characterized by having a secondary cytopenia (e.g. systemic autoimmune disease or malignancy) or Evans syndrome. In some embodiments, the autoantibody-related disease or disorder is a dermatologic disease or disorder. In some embodiments, the autoantibody-related disease or disorder is an autoimmune bullous dermatosis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

The TACI-Fc fusion protein may be provided in any of the formulations discussed herein. For example, in some embodiments, the TACI-Fc fusion protein is provided in a formulation comprising having a pH of from about 4.3 to about 5.1, about 5 mM to about 15 mM of an acetate, a tonicifying agent comprising arginine at or about 135 mM to 165 mM such as at or about 140 mM to 150 mM or an arginine-hydrochloride at a concentration of from at or about 1.5% to about 4% (w/v), and a non-ionic surfactant at a concentration of at or about 0.002% to about 1.0% (w/v), or at or about 0.01% to about 0.2% (w/v). In some cases, the surfactant is poloxamer 188 at or about 0.05% to about 0.2% (w/v) such as at or about 0.1% (w/v). In other embodiments, the surfactant is polysorbate 80 at or about 0.01% to about 0.04%. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the arginine hydrochloride is L-arginine hydrochloride. In some embodiments the formulation comprises arginine-hydrochloride at a concentration of about 3.1%. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is provided in a formulation comprising having a pH of from about 4.3 to about 5.2, about 5 mM to about 15 mM of an acetate, proline at a concentration of from at or about 1.5% to about 4%, a non-ionic surfactant at a concentration of from about 0.002% to about 1.0% (w/v), and a salt. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the proline is L-proline. In some embodiments the proline is at a concentration of about 1.5% to about 4%. In some embodiments the proline is at a concentration of at or about 2%. In some embodiments the proline is at a concentration of at or about 3%. In some embodiments, the surfactant is at a concentration of from about 0.05 to about 0.2% (w/v), such as at or about 0.1% (w/v). In some embodiments, the surfactant is poloxamer 188. In some embodiments, the surfactant is at a concentration of from about 0.01% to about 0.04%. In some embodiments, the surfactant is polysorbate 80. In some embodiments the salt is sodium chloride (NaCl). In some embodiments the amount of salt in the formulation is at or about 40 mM to at or about 100 mM. In some embodiments the amount of NaCl in the formulation is at or about 40 mM to at or about 100 mM. In some embodiments, the amount of salt, such as NaCl, is at or about 50 mM. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is administered intravenously. In some embodiments, the TACI-Fc fusion protein is administered subcutaneously.

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 4. Dermatologic Disease

In some embodiments, the provided TACI-Fc fusion proteins can be used for treating dermatologic diseases like autoimmune bullous (blistering) dermatosis (ABDs; also known as autoimmune blistering diseases). ABDs include pemphigus vulgaris and pemphigus foliaceus, among others. ABDs also include epidermolysis bullosa acquisita (EBA).

ABDs reflect autoantibodies targeting structural skin proteins. Treatments are limited to rituximab, which is the only biologic approved for pemphigus vulgaris, but may be associated with frequent relapses, often accompanied by elevations in the cytokine BAFF. BAFF and its related cytokine APRIL play key roles in B-cell activation across a broader spectrum of B cells than rituximab and are elevated in ABDs, correlating with disease activity. BAFF/APRIL inhibition may lead to more durable autoantibody reductions, improving clinical outcomes. The provided TACI-Fc fusion proteins provide an engineered BAFF/APRIL inhibitor that is more potent preclinically than other BAFF and/or APRIL inhibitors evaluated. Some are approved and/or demonstrate promising potential for related diseases like lupus, particularly its cutaneous manifestations.

In some aspects, provided herein is a method of treating autoimmune bullous (blistering) dermatosis (ABDs) comprising administering the TACI-Fc fusion proteins provided herein. In some embodiments, the method comprises: a) selecting a subject for administration of a TACI-Fc fusion protein that has been diagnosed with an autoimmune bullous (blistering) dermatosis; and b) administering to the selected subject the TACI-Fc fusion protein in accord with provided methods and doses (e.g. described in Section V. B), wherein: the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the Fc comprises the amino acid sequence set forth in SEQ ID NO:73. In some embodiments, the TACI-Fc fusion protein comprises the sequence of amino acids set forth in SEQ ID NO. 167. In some embodiments, the TACI-Fc fusion protein consists of the sequence of amino acids set forth in SEQ ID NO. 167. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section V.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the subject is selected for treatment if at the time of screening the subject has active blistering disease. In some embodiments, the subject has active blistering disease if the subject has visible blisters.

In some embodiments, the autoimmune bullous (blistering) dermatosis is selected from the group consisting of Pemphigus vulgaris, Pemphigus foliaceus or Bullous Pemphigoid. In some embodiments, the autoimmune bullous (blistering) dermatosis is Pemphigus vulgaris or Pemphigus foliaceus and the subject is selected for treatment if at the time of screening the subject is characterized by one or both of the following: (i) a Pemphigus Disease Area Index (PDAI) ≥15; and (ii) positive anti-Dsg1 or positive anti-Dsg3 antibodies.

In some embodiments, the autoimmune bullous (blistering) dermatosis is Pemphigus vulgaris.

In some embodiments, the autoimmune bullous (blistering) dermatosis is Pemphigus foliaceus.

In some embodiments, the autoimmune bullous (blistering) dermatosis is Pemphigoid and the subject is selected for treatment if at the time of screening the subject is characterized by one or both of the following: (i) IgA antibodies; and (ii) positive anti-Bp180 or positive anti-Bp230 antibodies.

In some embodiments, the subject is selected for treatment if at the time of screening or the time of administering the TACI-Fc fusion protein, the subject is receiving a stable immunosuppression. In some embodiments, the TACI-Fc fusion protein is administered to the subject in combination with concurrent administration of the stable immunosuppression.

In some embodiments, the stable immunosuppression comprises a stable dose of a steroid, such as a corticosteroid, for at least two weeks prior to the time of screening or the time of administering the TACI-Fc fusion protein; and/or the stable immunosuppression comprises a stable dose of azathioprine, MMF, or cyclosporine for at least four weeks prior to the time of screening or the time of administering the TACI-Fc fusion protein. In some embodiments, the stable immunosuppression comprises a stable dose of a calcineurin inhibitor (e.g., cyclosporine or tacrolimus) for at least four weeks prior to the time of screening or the time of administering the TACI-Fc fusion protein.

In some embodiments, the subject is not characterized by having a secondary disease (e.g. paraneoplastic). In some embodiments, the autoantibody-related disease or disorder is a neurologic disease or disorder. In some embodiments, the autoantibody-related disease or disorder is Encephalitis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, during the course of the treatment, the subject may be monitored for one or more endpoints of the disease to assess efficacy and/or one or more for an adverse event. In some embodiments, efficacy is monitored by disease control (DC). In some embodiments, efficacy is measured by partial remission (PR). In some embodiments, efficacy is monitored by complete remission (CR).

The TACI-Fc fusion protein may be provided in any of the formulations discussed herein. For example, in some embodiments, the TACI-Fc fusion protein is provided in a formulation comprising having a pH of from about 4.3 to about 5.1, about 5 mM to about 15 mM of an acetate, a tonicifying agent comprising arginine at or about 135 mM to 165 mM such as at or about 140 mM to 150 mM or an arginine-hydrochloride at a concentration of from at or about 1.5% to about 4% (w/v), and a non-ionic surfactant at a concentration of at or about 0.002% to about 1.0% (w/v), or at or about 0.01% to about 0.2% (w/v). In some cases, the surfactant is poloxamer 188 at or about 0.05% to about 0.2% (w/v) such as at or about 0.1% (w/v). In other embodiments, the surfactant is polysorbate 80 at or about 0.01% to about 0.04%. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the arginine hydrochloride is L-arginine hydrochloride. In some embodiments the formulation comprises arginine-hydrochloride at a concentration of about 3.1%. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is provided in a formulation comprising having a pH of from about 4.3 to about 5.2, about 5 mM to about 15 mM of an acetate, proline at a concentration of from at or about 1.5% to about 4%, a non-ionic surfactant at a concentration of from about 0.002% to about 1.0% (w/v), and a salt. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the proline is L-proline. In some embodiments the proline is at a concentration of about 1.5% to about 4%. In some embodiments the proline is at a concentration of at or about 2%. In some embodiments the proline is at a concentration of at or about 3%. In some embodiments, the surfactant is at a concentration of from about 0.05 to about 0.2% (w/v), such as at or about 0.1% (w/v). In some embodiments, the surfactant is poloxamer 188. In some embodiments, the surfactant is at a concentration of from about 0.01% to about 0.04%. In some embodiments, the surfactant is polysorbate 80. In some embodiments the salt is sodium chloride (NaCl). In some embodiments the amount of salt in the formulation is at or about 40 mM to at or about 100 mM. In some embodiments the amount of NaCl in the formulation is at or about 40 mM to at or about 100 mM. In some embodiments, the amount of salt, such as NaCl, is at or about 50 mM. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is administered intravenously. In some embodiments, the TACI-Fc fusion protein is administered subcutaneously.

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 5. Neurologic Disease

In some embodiments, the provided TACI-Fc fusion proteins can be used for treating neurologic disease like encephalitis. In some embodiments, encephalitis comprises autoimmune encephalitis or limbic encephalitis. In other embodiments, the neurologic disease comprises chronic inflammatory demyelinating polyneuropathy (CIDP), acute inflammatory demyelinating polyneuropathy (AIDP), multiple sclerosis, myasthenia gravis, or multifocal motor neuropathy (MVIN). In other embodiments, the neurologic disease comprises Neuromyelitis optica spectrum disorder (NMSOD).

In some aspects, provided herein is a method of treating encephalitis comprising administering the TACI-Fc fusion proteins provided herein. In some embodiments, the method comprises: a) selecting a subject for administration of a TACI-Fc fusion protein that has been diagnosed with Encephalitis; and b) administering to the selected subject the TACI-Fc fusion protein in accord with provided methods and doses (e.g. described in Section VI.B), wherein: the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the Fc comprises the amino acid sequence set forth in SEQ ID NO:73. In some embodiments, the TACI-Fc fusion protein comprises the sequence of amino acids set forth in SEQ ID NO. 167. In some embodiments, the TACI-Fc fusion protein consists of the sequence of amino acids set forth in SEQ ID NO. 167. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section V.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg Q4W. In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the encephalitis is autoimmune encephalitis. In some embodiments, the encephalitis is limbic encephalitis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, during the course of the treatment, the subject may be monitored for one or more endpoints of the disease to assess efficacy and/or one or more for an adverse event. In some embodiments, efficacy is monitored by modified Rankin Scale (mRS). In some embodiments, efficacy is measured by clinical assessment scale of autoimmune encephalitis (CASE).

The TACI-Fc fusion protein may be provided in any of the formulations discussed herein. For example, in some embodiments, the TACI-Fc fusion protein is provided in a formulation comprising having a pH of from about 4.3 to about 5.1, about 5 mM to about 15 mM of an acetate, a tonicifying agent comprising arginine at or about 135 mM to 165 mM such as at or about 140 mM to 150 mM or an arginine-hydrochloride at a concentration of from at or about 1.5% to about 4% (w/v), and a non-ionic surfactant at a concentration of at or about 0.002% to about 1.0% (w/v), or at or about 0.01% to about 0.2% (w/v). In some cases, the surfactant is poloxamer 188 at or about 0.05% to about 0.2% (w/v) such as at or about 0.1% (w/v). In other embodiments, the surfactant is polysorbate 80 at or about 0.01% to about 0.04%. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the arginine hydrochloride is L-arginine hydrochloride. In some embodiments the formulation comprises arginine-hydrochloride at a concentration of about 3.1%. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is provided in a formulation comprising having a pH of from about 4.3 to about 5.2, about 5 mM to about 15 mM of an acetate, proline at a concentration of from at or about 1.5% to about 4%, a non-ionic surfactant at a concentration of from about 0.002% to about 1.0% (w/v), and a salt. In some embodiments, the formulation has a pH of about 4.6. In some embodiments, the formulation has a pH of about 4.7. In some embodiments, the acetate is sodium acetate. In some embodiments, the acetate, such a sodium acetate, is at a concentration of at or about 10 mM. In some embodiments, the proline is L-proline. In some embodiments the proline is at a concentration of about 1.5% to about 4%. In some embodiments the proline is at a concentration of at or about 2%. In some embodiments the proline is at a concentration of at or about 3%. In some embodiments, the surfactant is at a concentration of from about 0.05 to about 0.2% (w/v), such as at or about 0.1% (w/v). In some embodiments, the surfactant is poloxamer 188. In some embodiments, the surfactant is at a concentration of from about 0.01% to about 0.04%. In some embodiments, the surfactant is polysorbate 80. In some embodiments the salt is sodium chloride (NaCl). In some embodiments the amount of salt in the formulation is at or about 40 mM to at or about 100 mM. In some embodiments the amount of NaCl in the formulation is at or about 40 mM to at or about 100 mM. In some embodiments, the amount of salt, such as NaCl, is at or about 50 mM. In some embodiments, the amount of TACI-Fc fusion protein in the formulation is from about 100 mg/mL and about 200 mg/mL. In some embodiments, the amount of TACI-Fc fusion protein in the formulation is at or about 160 mg. In some embodiments, the amount of TACI-Fc fusion protein in the formulation is at or about 175 mg.

In some embodiments, the TACI-Fc fusion protein is administered intravenously. In some embodiments, the TACI-Fc fusion protein is administered subcutaneously.

In some embodiments, the TACI-Fc fusion protein is administered subcutaneously in an amount at or about 80 mg Q4W. In some embodiments, the TACI-Fc fusion protein is administered subcutaneously in an amount at or about 240 mg Q4W.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### VI. ARTICLES OF MANUFACTURE AND KITS

Also provided herein are articles of manufacture that comprise the pharmaceutical formulations described herein in suitable packaging. Suitable packaging for formulations described herein are known in the art, and include, for example, vials (such as sealed vials), vessels, ampules, bottles, jars, flexible packaging (e.g., sealed Mylar or plastic bags), and the like, as well as syringes (e.g., single dose syringes) or vessels to be used with an injection device or the like. These articles of manufacture may further be sterilized and/or sealed.

Further provided are kits comprising the pharmaceutical formulations (or articles of manufacture) described herein, which may further comprise instruction(s) on methods of using the formulations, such as uses described herein. The kits described herein may also include other materials desirable from a commercial and user standpoint, including other buffers, diluents, filters, needles, syringes, and package inserts with instructions for performing any methods described herein.

In some embodiments the formulation is stored in an injection device. In some embodiments the formulation is stored in an injection device that is a syringe or that is an autoinjector (AI). In some embodiments the injection device is an on-body device. Such injection devices can be used for storage or can be used for storage and administration. For example, the formulation can be stored in the injection device and then can be administered to the subject. In some embodiments, the injection device is for self-administration. In some embodiments, the injection device is for self-administration by subcutaneous administration.

In particular embodiments the formulation is stored in an injection device that contains 0.5 mL, 1 mL, 1.5 mL, 2 mL, 2.5 mL, or 3 mL or more or contains a volume between any of the foregoing of the TACI-Fc formulation. In some embodiments the formulation is stored in a 2 mL injection device. In some embodiments the formulation is stored in an injection device that is a syringe. In some embodiments, the working volume of the syringe can be any working volume. In some embodiments, the syringe body can have a working volume of at least about 1000 microliters (1 mL). In some embodiments the syringe body can have a working volume of at most about 10 mL, at most about 5 mL, at most about 2 mL, at most about 1 mL, or at most about 750 microliters. In some embodiments, the working volume of the syringe body is 1 mL. In some embodiments, the working volume of the syringe body is 2 mL. In some embodiments, the working volume of the syringe body is 3 mL. In some embodiments, the TACI-Fc formulation is stored in the injection device and then is administered from the same injection device. In some embodiments the syringe is contained in an autoinjector.

In some embodiments the injection device is a syringe or is an autoinjector. In embodiments herein, an autoinjector contains a reservoir for storage of the formulation. The working volume of the reservoir can be any working volume. In some embodiments, the reservoir can have a working volume of at least about 1000 microliters (1 mL). In some embodiments the reservoir can have a working volume of at most about 10 mL, at most about 5 mL, at most about 2 mL, at most about 1 mL, or at most about 750 microliters. In some embodiments, the working volume of the syringe body is 1 mL. In some embodiments, the working volume of the reservoir is 2 mL. In some embodiments, the working volume of the reservoir is 3 mL.

In some embodiments, the TACI-Fc formulation is stored in and/or administered from a prefilled syringe. In some embodiments, the formulation is stored in a prefilled syringe (PFS). In some embodiments the prefilled syringe is a glass syringe. In some embodiments the prefilled syringe is a polymer syringe. In some embodiments the autoinjector contains a volume of 0.2 mL to 2 mL, such as 1 mL or 2 mL.

In some embodiments, the TACI-Fc formulation is administered from an autoinjector. In some embodiments, the autoinjector contains a prefilled syringe. In some embodiments the prefilled syringe is a glass syringe. In some embodiments the prefilled syringe is a polymer syringe. In some embodiments the autoinjector contains a volume of 0.2 mL to 2 mL, such as 1 mL or 2 mL. In some embodiments the autoinjector administers a fixed dose of the TACI-Fc protein in the formulation. In some embodiments the needle diameter of the syringe in the autoinjector is a 29 G TW or larger. In some embodiments the needle of the syringe in the autoinjector is a 27G needle. In some embodiments the injection time for the autoinjector is less than 10 seconds, such as less than 9, 8, 7, 6, 5, 4 seconds. In some embodiments the needle insertion depth of the syringe needle in the autoinjector is between 5-8 mm, such as 5 mm, 6 mm, 7 mm, or 8 mm or any value between the foregoing. In particular embodiments the autoinjector is from Ypsomed®. In some embodiments, the autoinjector is a YpsoMate® autoinjector.

In some embodiments, the syringe or autoinjector reservoir can be manufactured of materials that are biocompatible (e.g., silicone that is USP Class VI), inert, DMSO compatible, and/or shatter resistant. In some embodiments, the syringe or autoinjector reservoir can be manufactured of borosilicate glass or cyclic olefin polymer (COP).

In some embodiments the container has an extractable volume that is less than the total volume in the container. In some embodiments the extractable volume is at or about 1%, at or about 2%, at or about 3%, at or about 4%, or at or about 5%, at or about 6%, at or about 7%, at or about 8%, at or about 9%, at or about 10%, at or about 11%, at or about 12%, at or about 13%, at or about 14%, at or about 15%, at or about 16%, at or about 17%, at or about 18%, at or about 19%, at or about 20%, at or about 21%, at or about 22%, at or about 23%, at or about 24%, at or about 25%, at or about 26%, at or about 27%, at or about 28%, at or about 29%, at or about 30%, less than the total volume in the container. In particular embodiments the container has an extractable volume of 0.8 mL/container with an overfill volume of 0.2 mL/vial, for a total volume of 1 mL. For example, in embodiments the glass vial has an extractable volume of 0.8 mL/container with an overfill volume of 0.2 mL/vial, for a total volume of 1 mL. In other embodiments the container has an extractable volume of 0.9 mL/container with an overfill volume of 0.1 mL/vial, for a total volume of 1 mL. In another example, the container containing the TACI-Fc formulation is a prefilled injection device containing an extractable volume of 0.8 mL/container with an overfill volume of 0.2 mL/vial, for a total volume of 1 mL. In another example, the container containing the TACI-Fc formulation is a prefilled injection device containing an extractable volume of 0.46 mL/container with an overfill volume of 0.06 mL/container, for a total volume of 0.52 mL. In another example, the container containing the TACI-Fc formulation is a prefilled injection device containing an extractable volume of 1.37 mL/container with an overfill volume of 0.13 mL/container, for a total volume of 1.5 mL. In embodiments, the container containing the TACI-Fc formulation is an injection device where the extractable volume is a portion of the total volume in the injection device, such as, for example, in the syringe in the injection device.

In some embodiments the formulation is stored in a single use container. In particular embodiments the formulation is stored in single vial. In some embodiments, the glass vial is a 1 mL, 1.5 mL, 2 mL, 2.5 mL, or 3 mL glass vial, or is a glass vial that contains a volume between any of the foregoing. In some embodiments the formulation is stored in a 2 mL glass vial. In some embodiments the formulation is stored in a Type I glass vial closed with a rubber stopper and sealed with an aluminum cap.

In some embodiments the formulation is stored in a Type I glass vial closed with a rubber stopper and sealed with an aluminum cap. In some embodiments the formulation is stored in a prefilled syringe.

### VII. EXAMPLES

The following examples are included for illustrative purposes only and are not intended to limit the scope of the invention.

### Example 1

### Assessing Impact of Formulation Components on Aggregation of TACI-Fc

Studies were carried out to assess impacts of formulation components on stability of a TACI-Fc fusion protein. In these examples, the TACI-Fc fusion protein contained amino acid substitutions K77E, F78Y and Y102D in the wildtype human TACI sequence containing only a single CRD (CRD2) as set forth in SEQ ID NO: 13 (corresponding to residues 68-110 as set forth in UniProt Accession No. 014836), which was fused to an immunoglobulin IgG1 Fc sequence containing the mutations L234A, L235E and G237A (by the Eu Index numbering system for immunoglobulin proteins; SEQ ID NO:73) via a peptide linker (GSGGGGS; SEQ ID NO: 74). The variant TACI CRD2 polypeptide is set forth in SEQ ID NO:26, and the TACI-Fc fusion polypeptide is set forth by the sequence in SEQ ID NO: 167.

TACI ECD (68-110) (SEQ ID NO: 13):
SLSCRKEQGKFYDHLLRDCISCASICGQHPKQCAYFCENKLRS

Recombinant variant Fc fusion proteins were produced from a CHO based stable cell line.

The purified TACI-Fc fusion protein is a homodimer of 2 identical TACI Fc-fusion polypeptide chains, each with a variant TACI CRD2 domain human Fc-fusion polypeptide set forth in SEQ ID NO:167, linked by covalent disulfide bonds.

To assess impact of pH and certain excipients on stability of the TACI-Fc fusion protein, stability studies were carried out with TACI-Fc fusion protein at 100 mg/mL comparing the effects of pH followed by the effect of different excipients sucrose, proline, proline-sodium chloride mixture and arginine hydrochloride on aggregation as measured by size exclusion chromatography (SEC). Studies were conducted over subsequent 6 week periods comparing the effect of pH 5.2 vs. 7.6 on the stability over time at multiple temperatures. Further studies were carried out comparing the stability in formulations of the above excipients at pH 5.2 during a 6 week period.

The studies indicated that a formulation at pH 5.2 showed a lower rate of aggregate formation than that at pH 7.6 at 2-8° C. while those formulations containing either proline and sodium chloride or arginine hydrochloride had the lowest rates of aggregate formation during incubation at 2-8° C. as measured by SEC.

### Example 2

### Assessing the Impact of Ionic Strength and pH on the Stability of a TACI-Fc Fusion Protein

Additional studies were carried out to assess impacts of formulation components on long-term stability of a TACI-Fc fusion protein by monitoring the effect of ionic strength and pH on the stability of the TACI-Fc fusion protein homodimer described in Example 1. The studies were carried out using proline and sodium chloride as excipients, based on the initial stability screening results in Example 1.

In these studies, formulations at one of five different pH values (pH 5.06, 5.21, 5.39, 5.60 or 5.91) were prepared. Different ratios of proline to sodium chloride were incorporated into the formulations (e.g., 100% proline: 0% sodium chloride; 67% proline: 33% sodium chloride; 33% proline: 67% sodium chloride; or 0% proline: 100% sodium chloride. 3% w/v proline corresponds to 100% and 150 mM sodium chloride corresponds to 100%). Each formulation also was designed to contain 160 mg/mL TACI-Fc fusion protein. Four of the pHs (Formulations 1-16) used 10 mM sodium acetate as the initial buffering agent, with the starting pH being the lowest pH accomplished by buffer exchanging the TACI-Fc fusion protein into 10 mM acetic acid, 3% w/v proline at 160 mg/mL, measured at pH 5.03. The remaining three, acetate-based formulations then had an incremental increase in pH to pH 5.5 (target pH 5.19, 5.34, 5.5). The fifth pH was at 6.0 and used 10 mM histidine as the buffering agent. Each formulation was calculated to be isotonic. Poloxamer 188 (Pluronic F68) at 0.100 w/v was used as the surfactant for all formulations.

A summary of the tested formulations is set forth in Table E1.

TABLE E1
Formulations

| | Formulation | |
|---|---|---|
| Formulation | 160 mg/mL TACI-Fc fusion protein + listed | |
| Designator | excipients | Acronym |
| 1 | 160 mg/mL TACI-Fc, 10 mM sodium acetate, 3% L- | A5.06Pro |
| | proline w/v, 0.1% poloxamer 188 w/v, pH 5.06 | |
| 2 | 160 mg/mL TACI-Fc, 10 mM sodium acetate, 2% L- | A5.06-2Pro1NaCl |
| | proline w/v, 50 mM sodium chloride, 0.1% poloxamer | |
| | 188 w/v, pH 5.06 | |
| 3 | 160 mg/mL TACI-Fc, 10 mM sodium acetate, 1% | A5.06-1Pro2NaCl |
| | Proline w/v, 100 mM sodium chloride, 0.1% poloxamer | |
| | 188 w/v, pH 5.06 | |
| 4 | 160 mg/mL TACI-Fc, 10 mM sodium acetate, 150 mM | A5.06NaCl |
| | sodium chloride, 0.1% poloxamer 188 w/v, pH 5.06 | |
| 5 | 160 mg/mL TACI-Fc, 10 mM sodium acetate, 3% L- | A5.21Pro |
| | proline w/v, 0.1% poloxamer 188 w/v, pH 5.21 | |
| 6 | 160 mg/mL TACI-Fc, 10 mM sodium acetate, 2% L- | A5.21-2Pro1NaCl |
| | proline w/v, 50 mM sodium chloride, 0.1% poloxamer | |
| | 188 w/v, pH 5.21 | |
| 7 | 160 mg/mL TACI-Fc, 10 mM sodium acetate, 1% L- | A5.21-1Pro2NaCl |
| | proline w/v, 100 mM sodium chloride, 0.1% poloxamer | |
| | 188 w/v, pH 5.21 | |
| 8 | 160 mg/mL TACI-Fc, 10 mM sodium acetate, 150 mM | A5.21NaCl |
| | sodium chloride, 0.1% poloxamer 188 w/v, pH 5.21 | |
| 9 | 160 mg/mL TACI-Fc, 10 mM sodium acetate, 3% L- | A5.39Pro |
| | proline w/v, 0.1% poloxamer 188 w/v, pH 5.39 | |
| 10 | 160 mg/mL TACI-Fc, 10 mM sodium acetate, 2% L- | A5.39-2Pro1NaCl |
| | proline w/v, 50 mM sodium chloride, 0.1% poloxamer | |
| | 188 w/v, pH 5.39 | |
| 11 | 160 mg/mL TACI-Fc, 10 mM sodium acetate, 1% L- | A5.39-1Pro2NaCl |
| | proline w/v, 100 mM sodium chloride, 0.1% poloxamer | |
| | 188 w/v, pH 5.39 | |
| 12 | 160 mg/mL TACI-Fc, 10 mM sodium acetate, 150 mM | A5.39NaCl |
| | sodium chloride, 0.1% poloxamer 188 w/v, pH 5.39 | |
| 13 | 160 mg/mL TACI-Fc, 10 mM sodium acetate, 3% L- | A5.60Pro |
| | proline w/v, 0.1% poloxamer 188 w/v, pH 5.60 | |
| 14 | 160 mg/mL TACI-Fc, 10 mM sodium acetate, 2% L- | A5.60-2Pro1NaCl |
| | proline w/v, 50 mM sodium chloride, 0.1% poloxamer | |
| | 188 w/v, pH 5.60 | |
| 15 | 160 mg/mL TACI-Fc, 10 mM sodium acetate, 1% L- | A5.60-1Pro2NaCl |
| | proline w/v, 100 mM sodium chloride, 0.1% poloxamer | |
| | 188 w/v, pH 5.60 | |
| 16 | 160 mg/mL TACI-Fc, 10 mM sodium acetate, 150 mM | A5.60NaCl |
| | sodium chloride, 0.1% poloxamer 188 w/v, pH 5.60 | |
| 17 | 160 mg/mL TACI-Fc, 10 mM histidine, 3% L-proline | H5.91Pro |
| | w/v, 0.1% poloxamer 188 w/v, pH 5.91 | |
| 18 | 160 mg/mL TACI-Fc, 10 mM histidine, 2% L-proline | H5.91-2Pro1NaCl |
| | w/v, 50 mM sodium chloride, 0.1% poloxamer 188 w/v, | |
| | pH 5.91 | |
| 19 | 160 mg/mL TACI-Fc, 10 mM histidine, 1% L-proline | H5.91-1Pro2NaCl |
| | w/v, 100 mM sodium chloride, 0.1% poloxamer 188 | |
| | w/v, pH 5.91 | |
| 20 | 160 mg/mL TACI-Fc, 10 mM histidine, 150 mM | H5.91NaCl |
| | sodium chloride, 0.1% poloxamer 188 w/v, pH 5.91 | |

### A. Formulation Preparation

Formulations 1-20 set forth in Table E1, containing the various excipients and at the listed pH after buffer exchange, were prepared as detailed below. To limit the number of different formulation buffer exchanges, four initial formulations containing sodium acetate were prepared and buffer exchanged; all other sodium acetate formulations were prepared from mixtures of these initial, buffer exchanged formulations. These four initial preparations (Formulations #1, 4, 13, 16, in Table E1, above) contained 1000% sodium chloride or 100% proline formulations at the lowest and highest pH. From these four buffer exchanged preparations, the material was mixed to hit the target pH and excipient levels listed in Table E1. For the histidine-based formulations (Formulations #17-20), two initial formulations were prepared and buffer exchanged containing 100% sodium chloride or 100% proline to create the mixture of histidine based, sodium chloride-proline formulations (Formulations #18-19).

TACI-Fc fusion protein purified as described in Example 1 was prepared to a final concentration of 10 mg/mL. To achieve a final concentration of 160 mg/mL in the formulation, the 10 mg/mL purified TACI-Fc fusion protein was buffer exchanged into the respective UF/DF formulation buffer, material was over concentrated, collected, and diluted to hit the target concentration of 160 mg/mL. The TACI-Fc fusion protein was concentrated using either Centricon® filters or both Centricon® filters and cassette filters. For the histidine formulations (Formulations 17-20), TACI-Fc fusion protein was buffer exchanged using a 15 mL 10 kD Centricon® filter (Amicon® Ultra-15 Centrifugal Filter Unit Ultracel-10 regenerated cellulose membrane from Sigma®, Cat. No. UFC901024D). For the acetate formulations (Formulations 1-16), protein was buffer exchanged using two, 10 kD, 88 cm² cassette filters (from EID Millipore, Cat. No. P3CO10C00) followed by concentrating in the Centricon® filters. After buffer exchange, the TACI-Fc fusion protein was over concentrated, collected, and diluted to a final concentration of 160 mg/mL.

The pH of the buffers used for buffer exchange with TACI-Fc fusion protein are summarized in Table E2, with the target values defined from a pH set point study.

TABLE E2
pH set points for the UF/DF buffers

| | UF/DF | Measured | Final Target |
|---|---|---|---|
| | Buffer pH | UF/DF Buffer | pH at 160 |
| Buffer Formulation | Target | pH | mg/mL |
| 10 mM histidine, 3% L-proline w/v | 5.68 | 5.68 | 6.00 |
| 10 mM histidine, 150 mM NaCl | 6.10 | 6.10 | 6.00 |
| 10 mM acetic acid, 3% L-proline w/v | 4.14 | 4.12 | 5.03 |
| 10 mM sodium acetate, 3% L-proline | 5.30 | 5.30 | 5.50 |
| w/v | | | |
| 10 mM sodium acetate, 150 mM NaCl | 4.93 | 4.93 | 5.03 |
| 10 mM sodium acetate, 150 mM NaCl | 5.56 | 5.56 | 5.50 |

The material was then mixed as needed to prepare a formulation at the target pH and with the excipient concentrations set forth in Table E1. Specifically, a 10% (w/w) poloxamer 188 (from BASF, Kolliphor® Bio 188, Cat. No. 50424596) stock solution was prepared by weighing 0.698 g poloxamer 188 in a 15 mL falcon tube and adding Milli-Q® purified water to a total weight of 6.98 g. The poloxamer 188 solution was then mixed by spinning on a tube carousel for at least 1 hour at room temperature. To achieve a 0.1% (w/v) poloxamer 188 final concentration in the protein formulation, 34.3 μL of the 10% poloxamer 188 stock solution was added to 3.4 mL of TACI-Fc fusion protein in the acetate-based formulations. For the histidine-based formulations, 32.3 μL of the 10% poloxamer stock solution was added to 3.2 mL of TACI-Fc fusion protein.

Following addition of the poloxamer 188, the pH and protein concentration of the 20 formulations were measured. The protein concentration was determined using a SoloVPE variable path length spectrophotometer manufactured by Repligen. Samples were analyzed by adding the undiluted TACI-Fc product into a disposable sample vessel. The quick slope function of the software was employed to calculate protein concentration using an extinction coefficient of 1.25 mL/(mg*cm).

The results show that protein concentration was measured at +2 mg/mL of the target concentration of 160 mg/mL, and within the +16 mg/mL specification (±10% of target concentration). The results of the pH and protein concentration measurements are set forth in Table E3, below.

TABLE E3
pH and Protein Concentration of Formulations

| | pH measurements | | | | | | Protein Concentration (mg/mL) | | | |
|---|---|---|---|---|---|---|---|---|---|---|
| | 100% | 67% Pro/ | 33% Pro/ | 100% | Delta | Median | 100% | 67% Pro/ | 33% Pro/ | 100% |
| Condition | Proline | 33% NaCL | 67% NaCl | NaCl | pH | pH | Proline | 33% NaCL | 67% NaCl | NaCl |
| Acetate- | 5.12 | 5.10 | 5.02 | 4.95 | 0.18 | 5.06 | 159 | 160 | 160 | 160 |
| Low pH | | | | | | | | | | |
| Acetate- | 5.28 | 5.25 | 5.18 | 5.11 | 0.17 | 5.21 | 159 | 160 | 160 | 158 |
| Mid pH-1 | | | | | | | | | | |
| Acetate- | 5.42 | 5.42 | 5.37 | 5.30 | 0.12 | 5.39 | 159 | 159 | 158 | 159 |
| Mid pH-2 | | | | | | | | | | |
| Acetate- | 5.62 | 5.63 | 5.58 | 5.51 | 0.11 | 5.60 | 158 | 159 | 158 | 156 |
| High pH | | | | | | | | | | |
| Histidine | 5.82 | 5.88 | 5.93 | 5.96 | 0.14 | 5.91 | 159 | 157 | 158 | 159 |

### B. Characterization of TACI-Fc in Different Formulations

The properties of the TACI-Fc in the various formulations were assessed to determine the impact of the formulation components and pH on TACI-Fc long-term stability. The 20 formulations set forth in Table E1 were assessed for stability at 2-8° C. and 30° C. for 8 weeks, at timepoints 2, 4, and 8 weeks. Additionally, viscosity was measured at time zero for all formulations and for Formulations #1, 2, 3, and 4 at the 8-week time point.

For experiments where the formulations containing the TACI-Fc were assessed after prolonged incubation at 2-8° C. or 30° C., samples were prepared as follows. Crystal zenith (CZ) microtiter plates (West, Micro Plate 96R) and associated mats (96M-Plate Mat-2F D 21-7S) were sterilized by autoclaving prior to use. The 20 TACI-Fc-formulations were aseptically filtered in a biosafety cabinet using a Millex Filter unit (Millipore, SLMPL25SS) attached to a 10 mL, silicone oil free syringe (Henke Sass Wolf, 4100-X00V0). After filtration, 100 μL of each formulation was added to the inner 60 wells of the CZ microtiter plates. A total of three wells were filled per formulation. The remaining outer perimeter wells of the plate were filled with 100 μL of Milli-Q® purified water. A total of 6 plates were filled to support the study.

The plates were sealed using the associated plate mat. The 2-8° C. samples were placed directly at temperature, as is. For the 30° C. samples, the plates were placed in a mylar bag (U-Line, S-6175), 1 mL of Milli-Q® purified water was added to the bottom of the bag, and then the mylar bag was sealed with a double heat seal. This was then immediately placed on stability at 30° C.

Samples were collected from the plates as described below at different timepoints. At time zero, material was measured as a single replicate from the filtered material that had not been plated. For the remaining time points, all samples are from the plates and have three replicates per plate per formulation.

### 1. Viscosity

The viscosity of the various formulations was assessed to determine the effect of pH and excipients at the protein concentration of 160 mg/mL on this attribute. A solution's viscosity can influence the injectability of a formulation. Viscosity was measured at time=0 for all formulations, and viscosity also was measured for Formulations 1-4 after 8 weeks at 4° C. and 30° C. to determine if a change in viscosity occurred after prolonged storage.

Samples from each formulation were measured in triplicate prior to addition of the surfactant to determine the viscosity of the solution. Viscosity was measured using a cone and plate Anton Paar Modular Compact Rheometer (MCR) series MCR 92 with a CP20-0.5 Measuring Cone (Cat. 5956). The viscosity of the formulation was measured for the protein samples by aliquoting 25 μL of formulation onto the plate, and samples were run at 20° C. across a range of shear rates (50-2000 1/s) using a Ramp Logarithmic profile of 17 points and a point density of 10 pt./dec. Viscosity was then calculated using the Anton Paar RheoCompass software and averaged across a range between 500 and 2000 1/s shear rate and are reported in centipoise (cP).

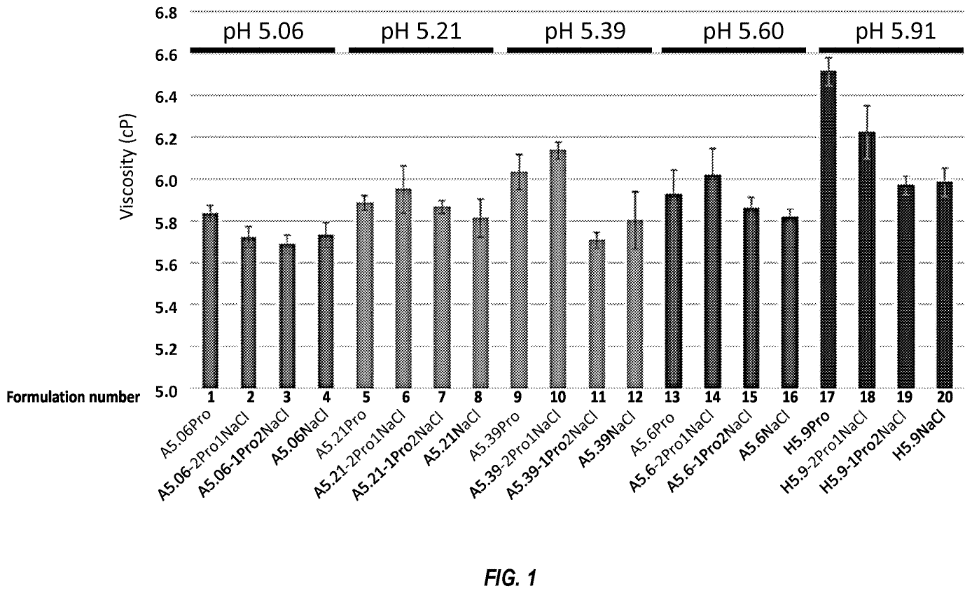

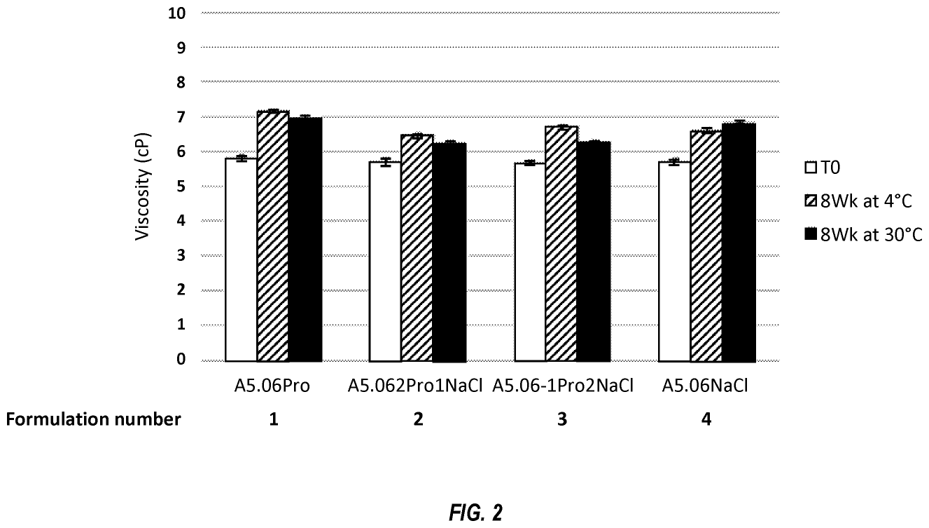

As set forth in Table E4 and FIG. 1, the viscosity measurements from the 20 formulations ranged from approximately 6.5 to 5.7 cP at time=0. For some formulations, there was an increase in viscosity at higher pH compared to the same formulation at a lower pH. For example, the formulations containing 10 mM sodium acetate, 200 L-proline w/v, 50 mM sodium chloride, and 0.10% poloxamer 188 w/v formulated at pH 5.06, pH 5.21, and pH 5.39 (Formulations 2, 6, and 10, respectively) had an average viscosity of 5.7, 6.0 and 6.1, respectively.

TABLE E4
Viscosity of formulations at 160/mg/mL at Time Zero

| | | Viscosity (cP) | | | | |
|---|---|---|---|---|---|---|
| Formulation | | | | | | Standard |
| Designator | Acronym | Rep 1 | Rep 2 | Rep 3 | Average | Deviation |
| 1 | A5.06Pro | 5.9 | 5.8 | 5.8 | 5.8 | 0.0 |
| 2 | A5.06-2Pro1NaCl | 5.7 | 5.7 | 5.8 | 5.7 | 0.1 |
| 3 | A5.06-1Pro2NaCl | 5.7 | 5.6 | 5.7 | 5.7 | 0.0 |
| 4 | A5.06NaCl | 5.7 | 5.8 | 5.7 | 5.7 | 0.1 |
| 5 | A5.21Pro | 5.9 | 5.9 | 5.9 | 5.9 | 0.0 |
| 6 | A5.21-2Pro1NaCl | 6.1 | 5.9 | 5.9 | 6.0 | 0.1 |
| 7 | A5.21-1Pro2NaCl | 5.9 | 5.9 | 5.8 | 5.9 | 0.0 |
| 8 | A5.21NaCl | 5.8 | 5.7 | 5.9 | 5.8 | 0.1 |
| 9 | A5.39Pro | 6.1 | 6.1 | 5.9 | 6.0 | 0.1 |
| 10 | A5.39-2Pro1NaCl | 6.2 | 6.2 | 6.1 | 6.1 | 0.0 |
| 11 | A5.39-1Pro2NaCl | 5.7 | 5.7 | 5.8 | 5.7 | 0.0 |
| 12 | A5.39NaCl | 5.9 | 5.7 | 5.9 | 5.8 | 0.1 |
| 13 | A5.60Pro | 5.8 | 6.1 | 5.9 | 5.9 | 0.1 |
| 14 | A5.60-2Pro1NaCl | 5.9 | 6.1 | 6.0 | 6.0 | 0.1 |
| 15 | A5.60-1Pro2NaCl | 5.8 | 5.8 | 5.9 | 5.9 | 0.1 |
| 16 | A5.60NaCl | 5.8 | 5.9 | 5.8 | 5.8 | 0.0 |
| 17 | H5.91Pro | 6.6 | 6.5 | 6.5 | 6.5 | 0.1 |
| 18 | H5.91-2Pro1NaCl | 6.3 | 6.3 | 6.1 | 6.2 | 0.1 |
| 19 | H5.91-1Pro2NaCl | 5.9 | 6.0 | 6.0 | 6.0 | 0.0 |
| 20 | H5.91NaCl | 6.0 | 6.1 | 5.9 | 6.0 | 0.1 |

### 2. Size Exclusion Chromatography (SEC)

SEC was used to assess stability and the rate of aggregation and the forms of the TACI-Fc protein in the various formulations. The SEC assay measures the total percentage of high molecular weight (HMW) and low molecular weight (LMW) species in each formulation to determine how the formulation components affect aggregation and stability of the TACI-Fc. SEC separates molecules based upon hydrodynamic radius which correlates with the molecular weight of an analyte. TACI-Fc typically assembles as a dimer consisting of two identical receptor Fc fusion polypeptide chains, each with a variant TACI domain-Fc fusion polypeptide chain, where the identical chains in the pair are linked by covalent disulfide bonds. In a formulation of purified TACI-Fc, the majority (up to 1000) of TACI-Fc in the formulation is a homodimer of the two TACI-Fc fusion proteins (referred to as the ‘main species,’ or simply ‘main’ in the chromatographic analyses provided herein). Smaller analytes are retained on the column longer and elute at later retention times. High molecular weight (HMW) species are defined as species eluting before the main species. The terminology and characterization of the different species of TACI-Fc products in this analysis is summarized in Table E6.

TABLE E6
Glossary of Characterization Terms for TACI-Fc Products with reference
to the TACI-Fc fusion protein set forth in SEQ ID NO. 167

| | | Apparent | | |
|---|---|---|---|---|
| | | number of | | |
| | | TACI-Fc fusion | Calculated | |
| Species | | polypeptide | aglycosylated | |
| Designator | Description | chain(s) | molecular mass | |
| Main | TACI-Fc homodimer | 2 | 62.6 | kDa |
| HMW | TACI-Fc species with a | >2 | >62.6 | kDa |
| | greater mass than the | | | |
| | TACI-Fc homodimer | | | |
| | (includes combination | | | |
| | of putative dimers and | | | |
| | oligomers) | | | |
| LMW | TACI-Fc species with | <2 | <62.6 | kDa |

lower mass than the
TACI-Fc homodimer,
elutes after the main
peak

Using SEC, the percentage of TACI-Fc HMW, LMW and main species was assessed. The rate of change of a species was calculated using linear regression by the least squares fit method of the data beginning with time 0. For SEC analysis, a formulation sample was injected onto an analytical column connected to an HPLC system and monitored by ultraviolet detection. At time zero, material was measured as a single replicate from the filtered material that had not been plated. For the remaining time points, all samples were from the plates and had three replicates per plate per formulation.

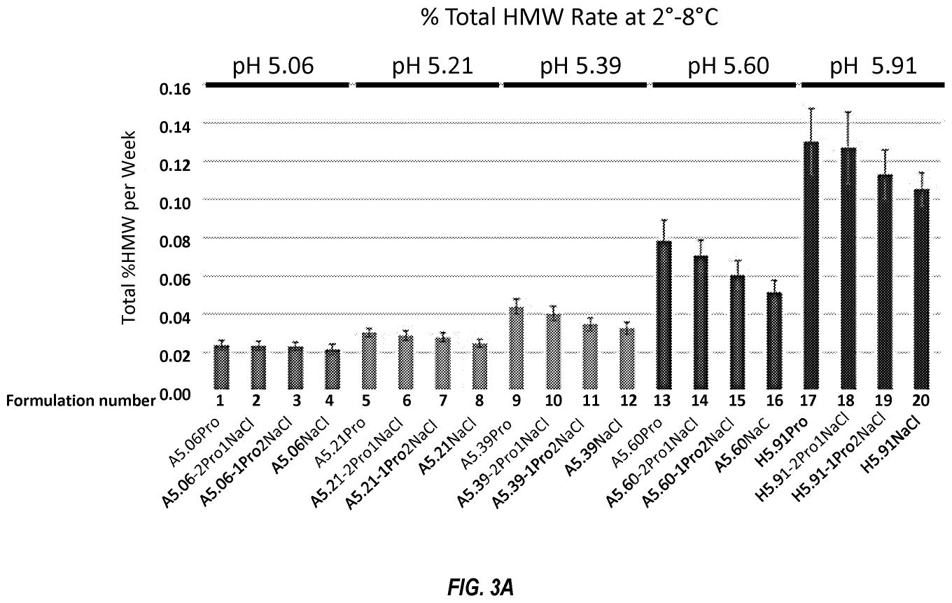

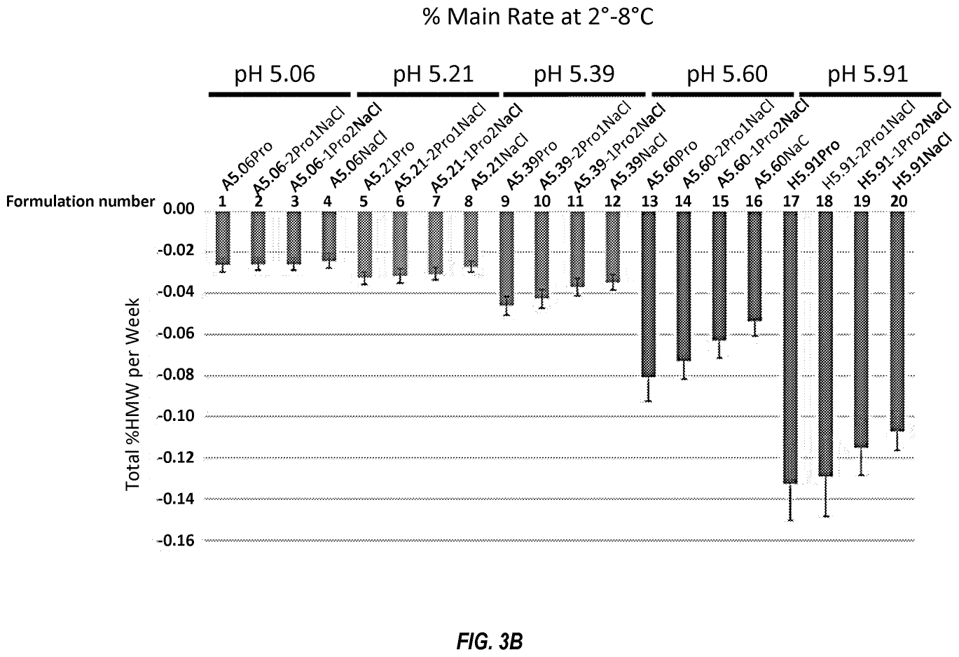

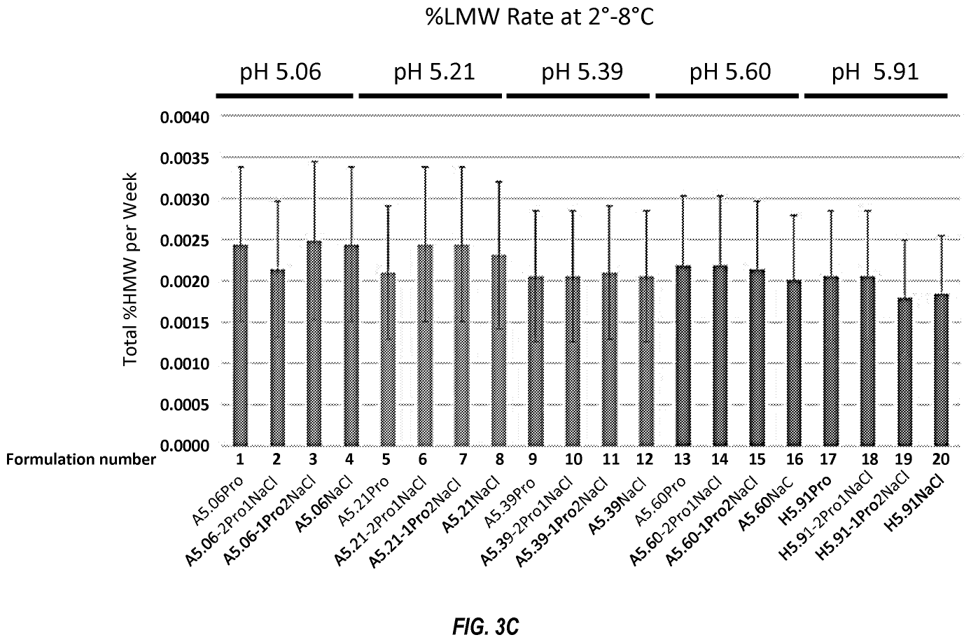

In the long term-stability study, at 2-8° C., there was a pH-dependent impact on the size variant stability trend rates as shown in FIG. 3A (% total HMW) and FIG. 3B (% main rate). There was no discernible trend for LMW formation, as shown in FIG. 3C (% LMW). The results show a pH dependent change in the rate of HMW formation across the formulations in the order of 5.91>5.6>5.39>5.21>5.06. Additionally, a sodium chloride dependent effect on stability was observed in formulations at pH 5.39 and pH 5.60, where the formulations showed greater stability with increasing sodium chloride concentrations within each pH range, leading to lower HMW species formation during storage.

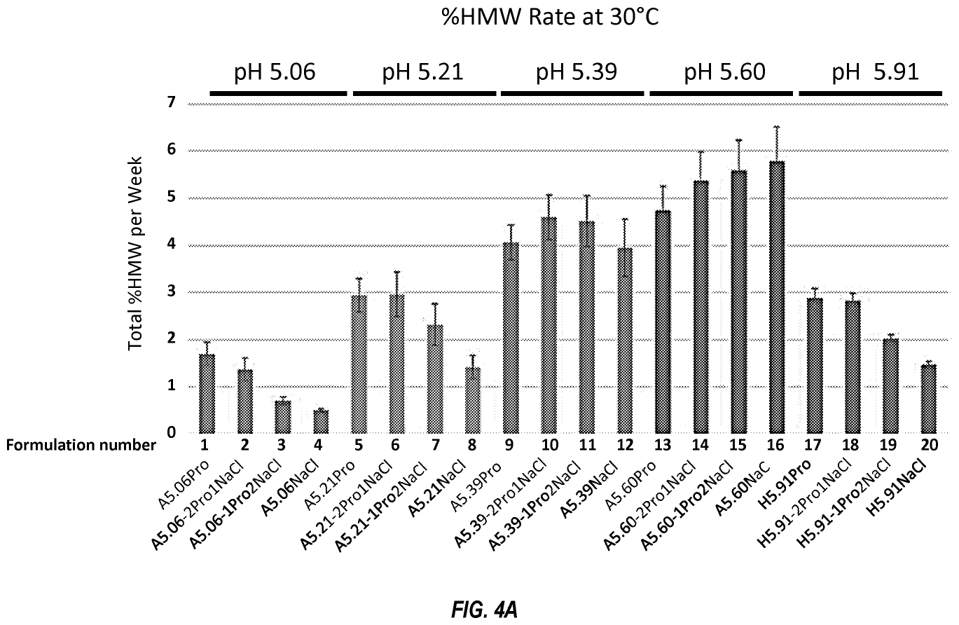

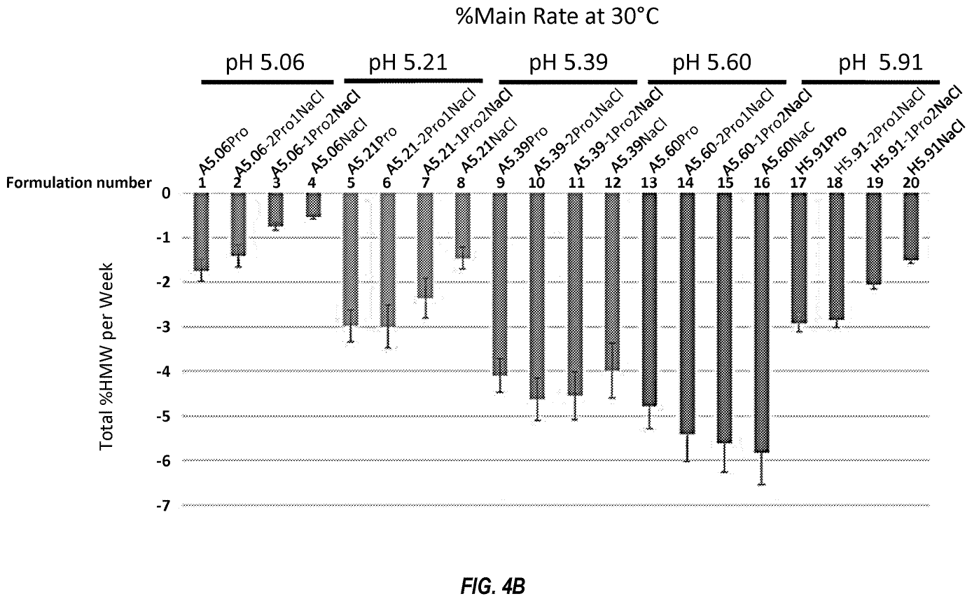

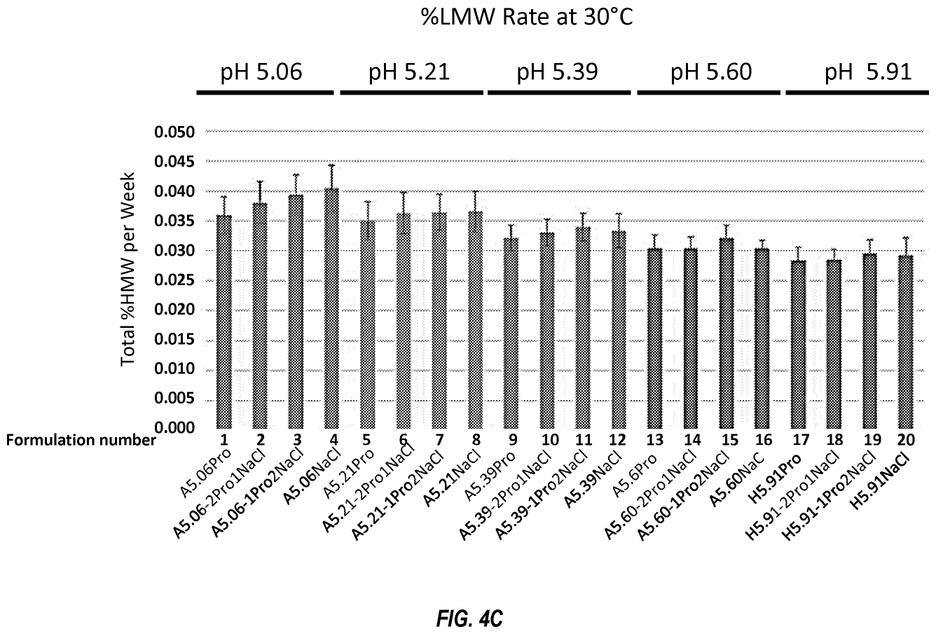

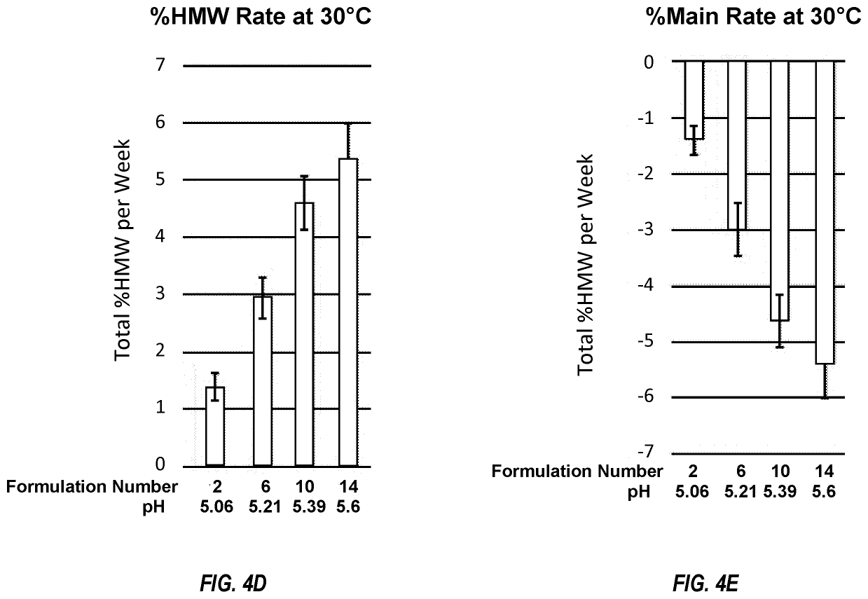

Results for the long-term stability study at 30° C. are shown in FIG. 4A (% total HMW), FIG. 4B (% main rate), and FIG. 4C (% LMW). Similar to the results at 2-8° C., a pH dependent effect on stability at 30° C. was also observed. The pH 5.06 formulations showed the lowest rates of degradation with the sodium chloride containing formulation showing the lowest overall rate of HMW species formation. In addition to the pH dependent effect there is an excipient dependent affect which is different at each pH shown. At pH 5.06 and 5.21, increasing the concentration of sodium chloride usually reduced the rate of aggregation while at pH 5.61 the opposite was true with the increasing sodium chloride increasing the rate of HMW species formation. This trend was discontinued for the histidine formulations at pH 5.91, which performed comparably to acetate at pH 5.21 acetate-based formulations. Formulation 2, which contained 160 mg/mL TACI-Fc, 10 mM sodium acetate, 2% L-proline w/v, 50 mM sodium chloride, 0.1% poloxamer 188 w/v formulated at pH 5.06, had a reduced rate of reduction of the main species at 30° C. compared to formulations containing the same components but prepared at pH 5.21, 5.39 and 5.6, formulations 6, 10 and 14, respectively (FIG. 4D). The reduction in the percent main was also pH-dependent, with the greatest decrease at pH 5.6, followed by pH 5.39 and pH 5.21 and then pH 5.06.

### 3. Background Membrane Imaging (BMI)

Background membrane imaging (BMI) was used to measure subvisible particles for all samples after storage at 30° C. or 2-8° C. for 8 weeks. Particles were measured by capturing a background membrane image for each well of an Aura 96 well plate (Halo Labs). Briefly, material was pipetted per well and dried using the BMI vacuum manifold. Each well was then rinsed with Milli-Q® water before the plate was vacuumed, dried, and analyzed on the Aura 96 well plate. Samples were from plates and had three replicates per plate per formulation. Each sample was measured in triplicate, and an average of the three wells is reported, below.

The number of particles that were ≥2 μm or ≥10 μm or ≥25 μm, per container, were counted.

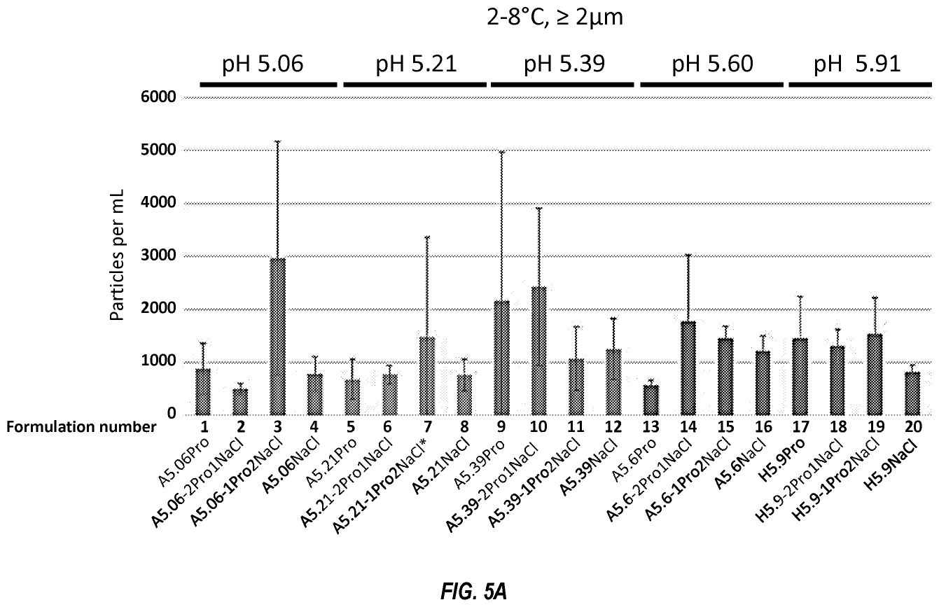

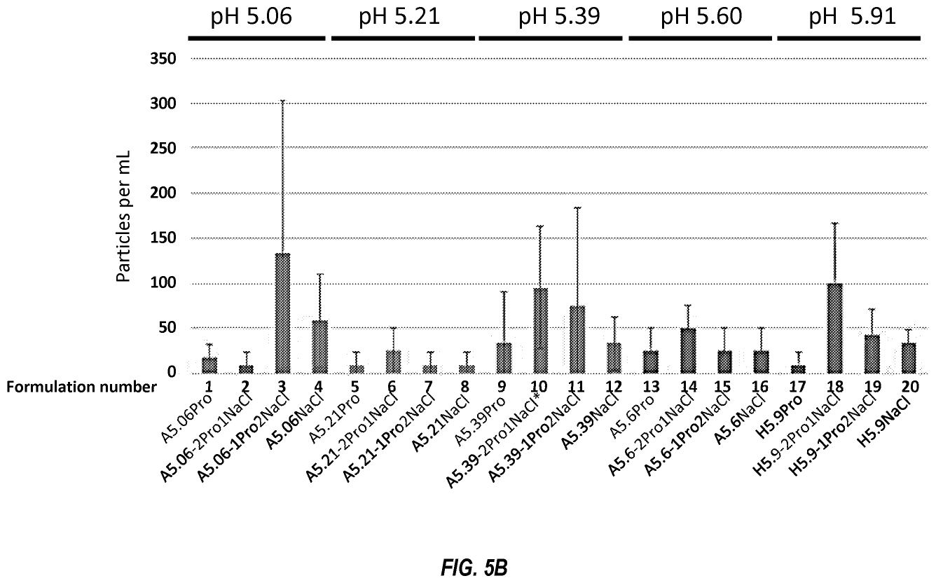

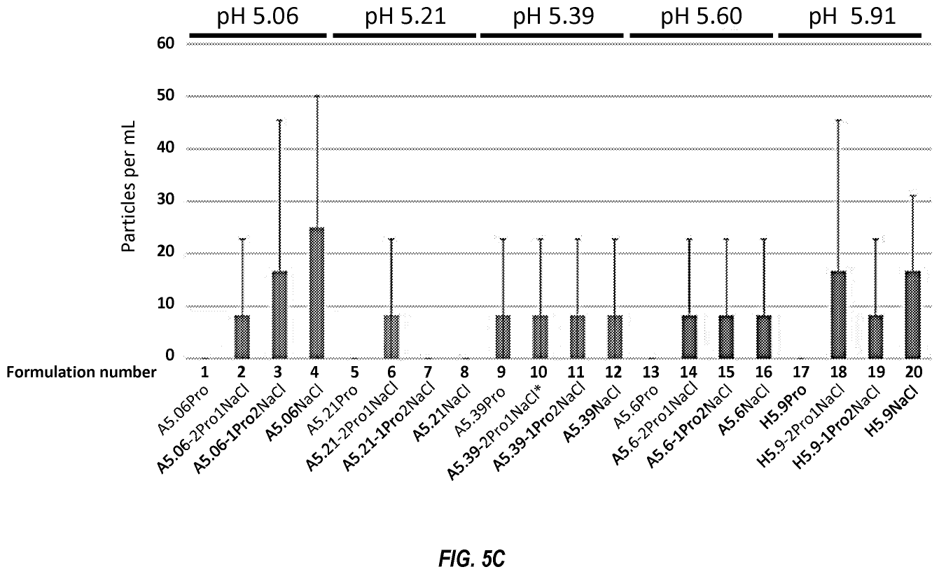

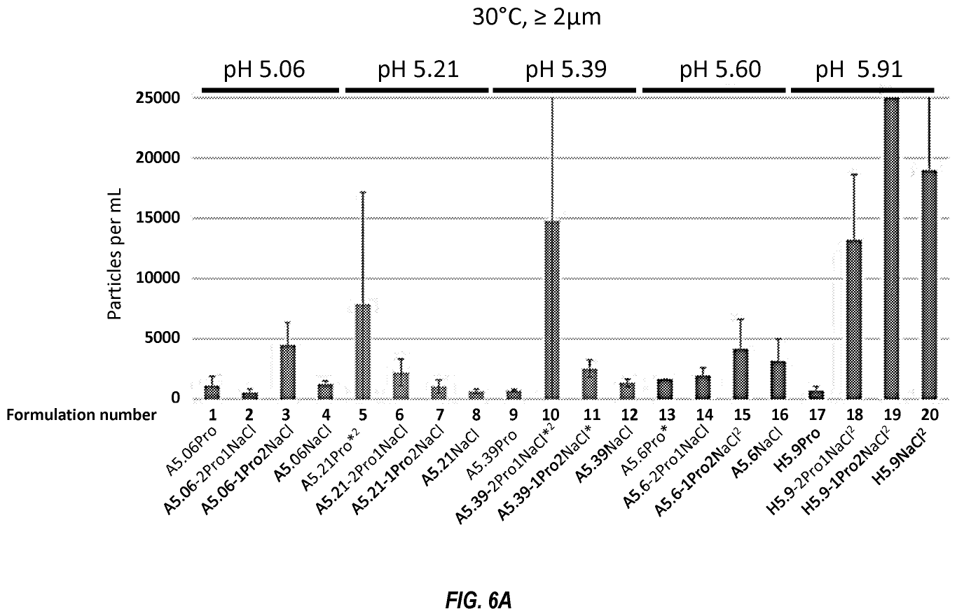

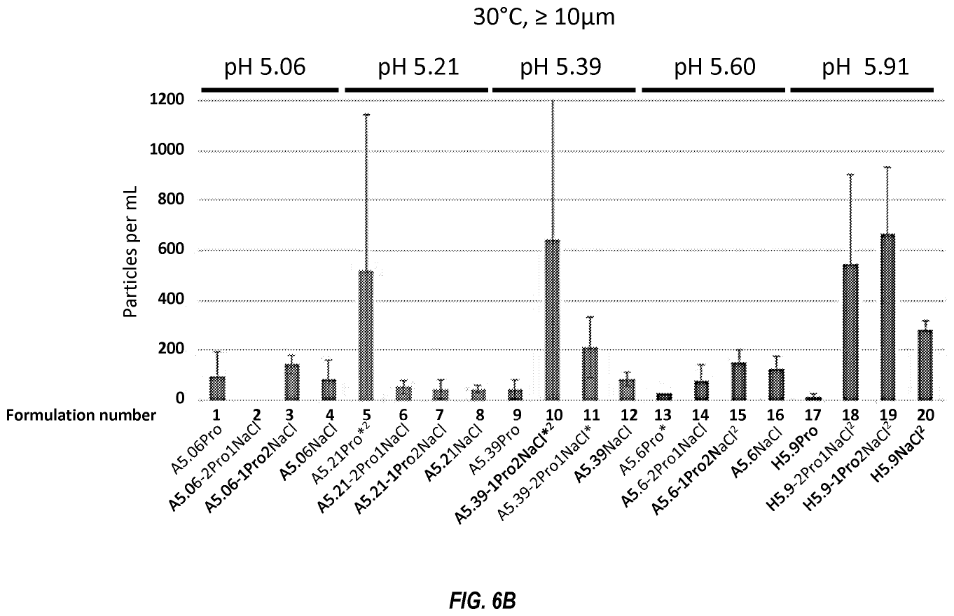

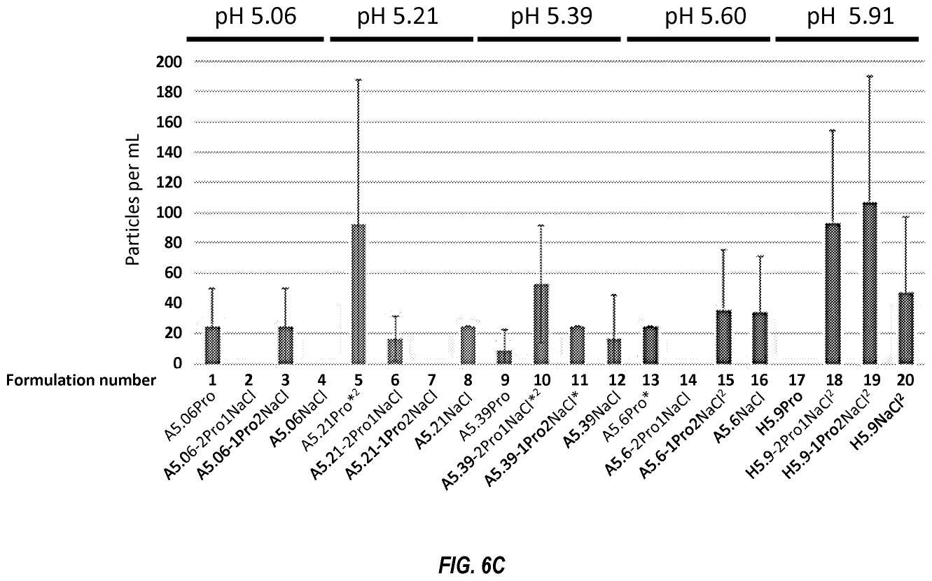

To help differentiate stability between formulations after long-term storage at 2-8° C., the final time point of eight weeks was graphed as set forth in FIGS. 5A-5C, for particles ≥2 μm, ≥10 μm, or ≥25 μm, respectively). A strong difference was not measured between the formulations for particles ≥2 μm and ≥10 μm. At ≥25 μm, the counts were also comparable across formulations. While not significant based on the error bars overlapping, there was a noticeable trend at pH 5.06, where the counts increased as the sodium chloride concentration increased. FIGS. 6A-6C show the particle counts by BMI after 8 weeks at 30° C., for particles ≥2 μm, ≥10 μm, or 25 μm, respectively. There was no distinct trend observed for the acetate containing formulations at any of the particle sizes measured. However, formulations containing histidine and sodium chloride and incubated at 30° C. showed a slight trend of higher particle counts for both particles ≥2 μm (FIG. 6A) or ≥10 μm (FIG. 6B) particle counts.

### 4. Qualitative Analysis

The individual wells were also qualitatively examined, to see if any particle trends could be observed based on the particle images from the BMI analysis. After two weeks of incubation at 2-8° C., qualitative analysis of particles showed no substantial difference in particle types between formulations. However, after incubation at 30° C., large, semitransparent particles were observed in the pH 5.06 acetate-sodium chloride formulation but were not observed in the pH 5.06 acetate-proline or acetate-proline-sodium chloride formulation. At four weeks, these particles were also observed at 4° C. in the pH 5.06 acetate-sodium chloride formulation, with multiple particles of this type present. These particles also were observed occasionally in other formulations but were generally more present at lower pH and higher sodium chloride concentration formulations. After 8 weeks at 4° C., it was observed that there were more larger and darker particles present for high sodium chloride formulations at pH 5.06 compared to what is generally seen in pure proline formulations. An increase in counts for particles ≥25 μm could be measured as the sodium chloride concentration increased due to these particles. These particles were not as prevalent at higher pH and were occasionally seen in other formulations. A standout difference between formulations was not observed after 8 weeks at 30° C.; the large, darker particles were observed randomly, throughout the formulations.

### 5. Summary

The results from Example 2, above, show that formulation 2, an acetate formulation containing 2% L-proline (w/v) and 50 mM sodium chloride at pH 5.06, showed stabilizing effects by SEC and had reduced large particles by BMI analysis. This formulation encompasses the most stabilizing pH by SEC and balances the use of proline with sodium chloride.

### Example 3

### Assessing the Impact of pH and Excipients Formulation on the Stability of a TACI-Fc Fusion Protein

The results in Example 2 show that formulation pH can impact the stability of the TACI-Fc fusion protein when assessed by viscosity, SEC and BMI. The presence of sodium chloride was also beneficial, lowering aggregation at the lower pH studied at 30° C. To expand on the results shown in Example 2, an additional study was carried out to further assess the impact of pH and different excipients on stability in formulations containing 160 mg/mL of TACI-Fc. The prepared formulations contained a variety of excipients and mixtures of excipients, including positively and negatively charged amino acids, in addition to mixtures of excipients with different stabilizing pathways, viscosity reducers, and self-buffering formulations.

Formulations with charged excipients were prepared at three different pH levels: 4.5, 5.1 and 5.5. A formulation that was very similar to formulation 2 from Example 2 containing 2% proline w/v, 50 mM NaCl at pH 5.06 in an acetate buffer, was included as a comparative sample (Formulation Designator 39 in Table E7, below). To explore different stabilizing pathways on protein stability from the effect of charged excipients, excipients such as alanine, caffeine, and glycine were studied at a singular pH of 5.1. All formulations, except the formulations containing only aspartic acid (#4-6), were measured to be isotonic. A summary of the tested formulations is set forth in Table E7.

TABLE E7
Further Formulations

| Formulation | Formulation | |
|---|---|---|
| Designator | 160 mg/mL TACI-Fc homodimer + listed excipients | Acronym |
| 21 | 160 mg/mL TACI-Fc, 10 mM sodium acetate, 3.1% L- | A4.5ArgPl.1 |
| | arginine hydrochloride w/v, 0.1% poloxamer 188 w/v, pH 4.5 | |
| 22 | 160 mg/mL TACI-Fc, 10 mM sodium acetate, 3.1% L- | A5.1ArgPl.1 |
| | arginine hydrochloride w/v, 0.1% poloxamer 188 w/v, pH 5.1 | |
| 23 | 160 mg/mL TACI-Fc, 160 mg/mL TACI-Fc, 10 mM sodium | A5.5ArgPl.1 |
| | acetate, 3.1% L-arginine hydrochloride w/v, 0.1% poloxamer | |
| | 188 w/v, pH 5.5 | |
| 24 | 160 mg/mL TACI-Fc, 10 mM acetic acid, 0.4% L-aspartic | A4AAPl.1 |
| | acid w/v, 0.1% poloxamer 188 w/v, pH 4 | |
| 25 | 160 mg/mL TACI-Fc, 10 mM sodium acetate, 0.4% L | A4.1AAPl.1 |
| | aspartic acid w/v, 0.1% poloxamer 188 w/v, pH 4.1 | |
| 26 | 160 mg/mL TACI-Fc, 20 mM sodium acetate, 0.4% L- | A4.3AAPl.1 |
| | aspartic acid w/v, 0.1% poloxamer 188 w/v, pH 4.3 | |
| 27 | 160 mg/mL TACI-Fc, 10 mM sodium acetate, 1% L-proline | A4.5ProSorbNaClPl.1 |
| | w/v, 1.6% sorbitol w/v, 50 mM NaCl, 0.1% poloxamer 188 | |
| | w/v, pH 4.5 | |
| 28 | 160 mg/mL TACI-Fc, 10 mM sodium acetate, 1% L-proline | A5.1ProSorbNaClPl.1 |
| | w/v, 1.6% sorbitol w/v, 50 mM NaCl, 0.1% poloxamer 188 | |
| | w/v, pH 5.1 | |
| 29 | 160 mg/mL TACI-Fc, 10 mM sodium acetate, 1% L-proline | A5.5ProSorbNaClPl.1 |
| | w/v, 1.6% sorbitol w/v, 50 mM NaCl, 0.1% poloxamer 188 | |
| | w/v, pH 5.5 | |
| 30 | 160 mg/mL TACI-Fc, 10 mM sodium acetate, 1.5% L-proline | A4.5ProArgPl.1 |
| | w/v, 1.55% L-arginine hydrochloride w/v, 0.1% poloxamer | |
| | 188 w/v, pH 4.5 | |
| 31 | 160 mg/mL TACI-Fc, 10 mM sodium acetate, 1.5% L-proline | A5.1ProArgPl.1 |
| | w/v, 1.55% L-arginine hydrochloride w/v, 0.1% poloxamer | |
| | 188 w/v, pH 5.1 | |
| 32 | 160 mg/mL TACI-Fc, 10 mM sodium acetate, 1.5% L-proline | A5.5ProArgPl.1 |
| | w/v, 1.55% L-arginine hydrochloride w/v, 0.1% poloxamer | |
| | 188 w/v, pH 5.5 | |
| 33 | 160 mg/mL TACI-Fc, 10 mM acetic acid, 2.7% L-proline | A4.2ProAAPl.1 |
| | w/v, 0.4% L-aspartic acid w/v, 0.1% poloxamer 188 w/v, pH | |
| | 4.2 | |
| 34 | 160 mg/mL TACI-Fc, 10 mM sodium acetate, 2.7% L-proline | A4.3ProAAPl.1 |
| | w/v, 0.4% L-aspartic acid w/v, 0.1% poloxamer 188 w/v, pH | |
| | 4.3 | |
| 35 | 160 mg/mL TACI-Fc, 20 mM sodium acetate, 2.7% L-proline | A4.4ProAAPl.1 |
| | w/v, 0.4% L-aspartic acid w/v, 0.1% poloxamer 188 w/v, pH | |
| | 4.4 | |
| 36 | 160 mg/mL TACI-Fc, 10 mM acetic acid, 2.4% L-alanine | A5.1AlaPl.1 |
| | w/v, 0.1% poloxamer 188 w/v, pH 5.1 | |
| 37 | 160 mg/mL TACI-Fc, 10 mM sodium acetate, 28 mM | A5.1CafProNaClPl.1 |
| | caffeine, 1.7% L-proline w/v, 50 mM NaCl, 0.1% poloxamer | |
| | 188 w/v, pH 5.1 | |
| 38 | 160 mg/mL TACI-Fc, 10 mM acetic acid, 2% glycine w/v, | A5.1GlyPl.1 |
| | 0.1% poloxamer 188 w/v, pH 5.1 | |
| 39 | 160 mg/mL TACI-Fc, 10 mM sodium acetate, 2% L-proline | A5.1ProNaClPl.1 |
| | w/v, 50 mM sodium chloride, 0.1% poloxamer 188 w/v, pH | |
| | 5.1 | |
| 40 | 160 mg/mL TACI-Fc, 2% L-proline w/v, 50 mM sodium | Self-5.4ProNaClPl.1 |
| | chloride, 0.1% poloxamer 188 w/v, pH 5.1 | |

### A. Formulation Preparation

Formulations 21-40, listed in Table E7 containing the various excipients and at the listed pH, were prepared as detailed below. Purified TACI-Fc fusion protein described in Example 1 at 10 mg/mL was buffer exchanged using 15 mL, 10 kD Centricon® filters (Sigma, UFC901024D) for all formulations. After buffer exchange, material was over concentrated, collected, and diluted to hit the target concentration of 160 mg/mL. The pH of the UF/DF buffers are described in Table E8.

TABLE E8
pH Set points

| | | | Measured UF/DF | Final Target pH at |
|---|---|---|---|---|
| Designator | Formulation | UF/DF pH Target | buffer pH | 160 mg/mL |
| 21 | A4.5ArgPl.1 | 4.44 | 4.43 | 4.50 |
| 22 | A5.1ArgPl.1 | 5.10 | 5.10 | 5.06 |
| 23 | A5.5ArgPl.1 | 5.62 | 5.61 | 5.50 |
| 24 | A4AAPl.1 | Acid conjugate | 2.95 | 4.00 |
| 25 | A4.1AAPl.1 | Base conjugate (10 mM) | 3.52 | 4.10 |
| 26 | A4.3AAPl.1 | Base conjugate (20 mM) | 3.89 | 4.30 |
| 27 | A4.5ProSorbNaClPl.1 | 4.27 | 4.27 | 4.50 |
| 28 | A5.1ProSorbNaClPl.1 | 5.00 | 5.00 | 5.06 |
| 29 | A5.5ProSorbNaClPl.1 | 5.57 | 5.57 | 5.50 |
| 30 | A4.5ProArgPl.1 | 4.35 | 4.36 | 4.50 |
| 31 | A5.1ProArgPl.1 | 5.02 | 5.02 | 5.06 |
| 32 | A5.5ProArgPl.1 | 5.55 | 5.54 | 5.50 |
| 33 | A4.2ProAAPl.1 | Acid conjugate | 3.44 | 4.20 |
| 34 | A4.3ProAAPl.1 | Base conjugate (10 mM) | 3.75 | 4.30 |
| 35 | A4.4ProAAPl.1 | Base conjugate (20 mM) | 4.00 | 4.40 |
| 36 | A5.1AlaPl.1 | Acid conjugate | 4.36 | 5.06 |
| 37 | A5.1CafProNaClPl.1 | 4.99 | 4.99 | 5.06 |
| 38 | A5.1GlyPl.1 | Acid conjugate | 4.36 | 5.06 |
| 39 | A5.1ProNaClPl.1 | 4.99 | 4.98 | 5.06 |
| 40 | Self-5.4ProNaClPl.1 | — | 6.23 | — |

Each of the tested formulations shown in Table E7 were prepared to contain 0.1% poloxamer 188 w/v. To prepare the 0.10 poloxamer 188 w/v, a to (w/w) poloxamer 188 (BASF, Kolliphor® Bio 188, 50424596) stock solution was prepared by weighing 0.760 g poloxamer 188 in a 15 mL falcon tube and adding Milli-Q® purified water until the total weight was 7.62 g. A density of 1.0 g/mL was used for the 10% w/w poloxamer 188 stock solution. The poloxamer 188 solution was mixed by spinning on a tube carousel for at least 1 hour at room temperature. To achieve a 0.1% (w/v) poloxamer 188 final concentration in the protein formulation, the 10% w/w stock solution was added to the protein in the amounts described in Table E9, below.

TABLE E9
Surfactant Added

| | Formulation | | |
|---|---|---|---|
| Number | Designator | mL protein | μL poloxamer 188 to add |
| 21 | A4.5ArgPl.1 | 2.74 | 27.7 |
| 22 | A5.1ArgPl.1 | 3.26 | 32.9 |
| 23 | A5.5ArgPl.1 | 3.10 | 31.3 |
| 24 | A4AAPl.1 | 2.94 | 29.7 |
| 25 | A4.1AAPl.1 | 3.23 | 32.6 |
| 26 | A4.3AAPl.1 | 2.60 | 26.3 |
| 27 | A4.5ProSorbNaClPl.1 | 3.09 | 31.2 |
| 28 | A5.1ProSorbNaClPl.1 | 3.35 | 33.8 |
| 29 | A5.5ProSorbNaClPl.1 | 3.12 | 31.5 |
| 30 | A4.5ProArgPl.1 | 3.20 | 32.3 |
| 31 | A5.1ProArgPl.1 | 3.38 | 34.1 |
| 32 | A5.5ProArgPl.1 | 2.81 | 28.4 |
| 33 | A4.2ProAAPl.1 | 2.83 | 28.6 |
| 34 | A4.3ProAAPl.1 | 3.41 | 34.4 |
| 35 | A4.4ProAAPl.1 | 3.02 | 30.5 |
| 36 | A5.1AlaPl.1 | 2.92 | 29.5 |
| 37 | A5.1CafProNaClPl.1 | 2.99 | 30.2 |
| 38 | A5.1GlyPl.1 | 3.10 | 31.3 |
| 39 | A5.1ProNaClPl.1 | 3.29 | 33.2 |
| 40 | Self-5.4ProNaClPl.1 | 2.86 | 28.9 |

Following addition of the poloxamer 188, but before filtration and plating for the stability studies described below, the pH and protein concentration of the 20 formulations were measured generally as detailed in Example 2. The pH measurements are set forth in Table E10. For formulations without aspartic acid and containing a buffering agent, the target low, mid, and high pH were: pH 4.5, 5.06, and 5.5.

TABLE E10
pH measurements of Round 2 Formulations

| | | Target pH | | |
|---|---|---|---|---|
| | Formulation Designators | Low pH | Mid pH | High pH |
| Formulation Excipient | (formulation number) | (4.5) | (5.1) | (5.5) |
| Arginine | A4.5ArgPl.1 (21) | 4.55 | 5.15 | 5.50 |
| | A5.1ArgPl.1 (22) | | | |
| | A5.5ArgPl.1 (23) | | | |
| Aspartic Acid | A4AAPl.1 (24) | 3.96 | 4.13 | 4.30 |
| | A4.1AAPl.1 (25) | | | |
| | A4.3AAPl.1 (26) | | | |
| Proline-Sorbitol-NaCL | A4.5ProSorbNaClPl.1 (27) | 4.53 | 5.11 | 5.54 |
| | A5.1ProSorbNaClPl.1 (28) | | | |
| | A5.5ProSorbNaClPl.1 (29) | | | |
| Proline-Arginine | A4.5ProArgPl.1 (30) | 4.51 | 5.09 | 5.50 |
| | A5.1ProArgPl.1 (31) | | | |
| | A5.5ProArgPl.1 (32) | | | |
| Proline-Aspartic Acid | A4.2ProAAPl.1 (33) | 4.18 | 4.30 | 4.42 |
| | A4.3ProAAPl.1 (34) | | | |
| | A4.4ProAAPl.1 (35) | | | |
| Alanine | A5.1AlaPl.1 (36) | | 5.13 | |
| Caffeine-Proline-NaCl | A5.1CafProNaClPl.1 (37) | | 5.06 | |
| Glycine | A5.1GlyPl.1 (38) | | 5.13 | |
| Proline-NaCl | A5.1ProNaClPl.1 (39) | | 5.06 | |
| Self buffering-Proline-NaCL | Self-5.4ProNaClPl.1 (40) | | 5.44 | |

The protein concentration measurements are set forth in Table E11. The results show that protein concentration was measured at ±9 mg/mL of the target concentration of 160 mg/mL, and within the ±16 mg/mL specification (±1000 of target concentration).

TABLE E11
Protein Concentration of Round 2 Formulations

| | Formulation | Low | Mid | High |
|---|---|---|---|---|
| | Designators | pH | pH | pH |
| Formulation | (formulation number) | (4.5) | (5.1) | (5.5) |
| Arginine | A4.5ArgPl.1 (21) | 157 | 154 | 162 |
| | A5.1ArgPl.1 (22) | | | |
| | A5.5ArgPl.1 (23) | | | |
| Aspartic Acid | A4AAPl.1 (24) | 163 | 161 | 153 |
| | A4.1AAPl.1 (25) | | | |
| | A4.3AAPl.1 (26) | | | |
| Proline-Sorbitol-NaCL | A4.5ProSorbNaClPl.1 | 161 | 151 | 157 |
| | (27) | | | |
| | A5.1ProSorbNaClPl.1 | | | |
| | (28) | | | |
| | A5.5ProSorbNaClPl.1 | | | |
| | (29) | | | |
| Proline-Arginine | A4.5ProArgPl.1 (30) | 160 | 158 | 164 |
| | A5.1ProArgPl.1 (31) | | | |
| | A5.5ProArgPl.1 (32) | | | |
| Proline-Aspartic Acid | A4.2ProAAPl.1 (33) | 159 | 162 | 160 |
| | A4.3ProAAPl.1 (34) | | | |
| | A4.4ProAAPl.1 (35) | | | |
| Alanine | A5.1AlaPl.1 (36) | | 160 | |
| Caffeine-Proline-NaCl | A5.1CafProNaClPl.1 | | 163 | |
| | (37) | | | |
| Glycine | A5.1GlyPl.1 (38) | | 159 | |
| Proline-NaCl | A5.1ProNaClPl.1 (39) | | 153 | |
| Self buffering-Proline- | Self-5.4ProNaClPl.1 | | 159 | |
| NaCL | (40) | | | |

### B. Characterization of TACI-Fc in Different Formulations

The properties of the TACI-Fc in the various formulation were assessed to determine the impact of the formulation components and pH on TACI-Fc stability. Formulations 21-41 were assessed for stability by size exclusion chromatography (SEC) and background membrane imaging (BMI) after incubation at 2-8° C. or 30° C. for up to 8 weeks, at timepoint intervals of 2, 4, and 8 weeks. Additionally, viscosity was measured at time zero for all formulations.

For the long-term stability studies, crystal zenith (CZ) microtiter plates (West, Micro Plate 96R) and associated matts (96M-Plate Mat-2F7 D 21-7S) were sterilized by autoclaving prior to use. The 20 formulations were each aseptically filtered in a BSC using a Millex Filter unit (Millipore, SLMPL25SS) attached to a 10 mL, silicone oil free syringe (Henke Sass Wolf, 4100-X00V0). After filtration, 100 μL of each formulation was added in triplicate to the inner 60 wells of the CZ microtiter plates (three wells per formulation). The remaining outer perimeter wells of the plate that were not filled with the formulations were filled with 100 μL of Milli-Q® purified water and the plates were sealed using the associated plate mat.

Following sealing, the 2-8° C. samples were placed directly at temperature, and the plates containing the samples for incubation at 30° C. were placed in a mylar bag (U-Line, S-6175) and 1 mL of Milli-Q® purified water was added to the bottom of the bag, and the mylar bag was sealed with a double heat seal. The bag was then immediately placed on a stable surface at 30° C. and incubated for the appropriate amount of time, as detailed below.

### 1. Viscosity

Viscosity was measured at time=0 for all formulations at a TACI-Fc concentration of 160 mg/mL. Samples from each condition were measured in triplicate. Viscosity was measured as detailed in Example 2, above. A formulation that was negatively impacting would have a viscosity >5 cP difference between formulations while a viscosity of >15 cP would be considered high for manufacturability and injectability via a syringe.

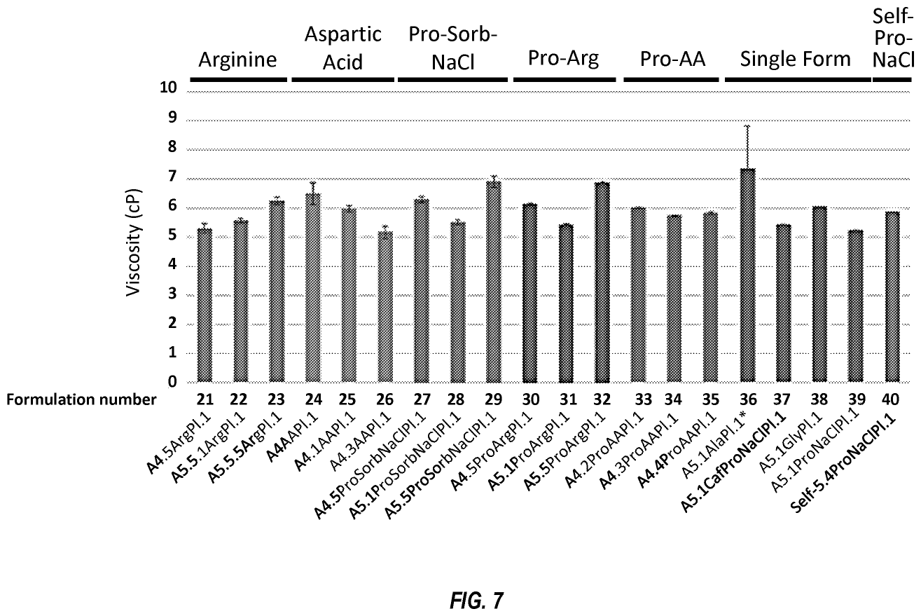

As set forth in FIG. 7, the viscosity measurements from the 20 formulations ranged from approximately 7.3 to 5.2 cP at time=0. For the alanine-based formulation (Formulation #16), two additional measurements were taken due to initial measurements displaying a wide range of viscosity; the two additional viscosity measurements were 6.4 cP and 6.4 cP. For some formulations, there was an increase in viscosity at higher pH compared to the same formulation at a lower pH. For example, the arginine (“Arg”) formulations containing 10 mM sodium acetate, 3.1% L-arginine hydrochloride w/v, 0.1% poloxamer 188 w/v had an average viscosity of 5.3 cP when formulated at pH 4.5 and an average viscosity of 6.3 cP when formulated at pH 5.5 (Formulation 21 compared to Formulation 23). A similar trend was seen for the formulations containing 10 mM sodium acetate, 1.5% L-proline w/v, 1.55% L-arginine hydrochloride, 0.1% poloxamer 188 w/v (formulations 30 and 32). The results show that the formulation conditions, including the formulation pH, can impact the viscosity of the formulation; in some formulations, preparing the formulation at a lower pH results in a lower viscosity of the formulation.

### 2. Size Exclusion Chromatography (SEC)

SEC was used to assess stability and the rate of aggregation and fragmentation of TACI-Fc protein in the various formulations as detailed in Example 2. The formulations were incubated at 2-8° C. or 30° C. for up to 8 weeks and the percentage of the formulation that was an HMW, LMW, or main species was assessed. The rate of change in the percent of the different species in the formulation was calculated using linear regression by the least squares fit method of the data beginning with time 0. At time zero, material was measured as a single replicate from the filtered material that had not been plated. For the remaining time points, all samples were from the plates and had three replicates per plate per formulation.

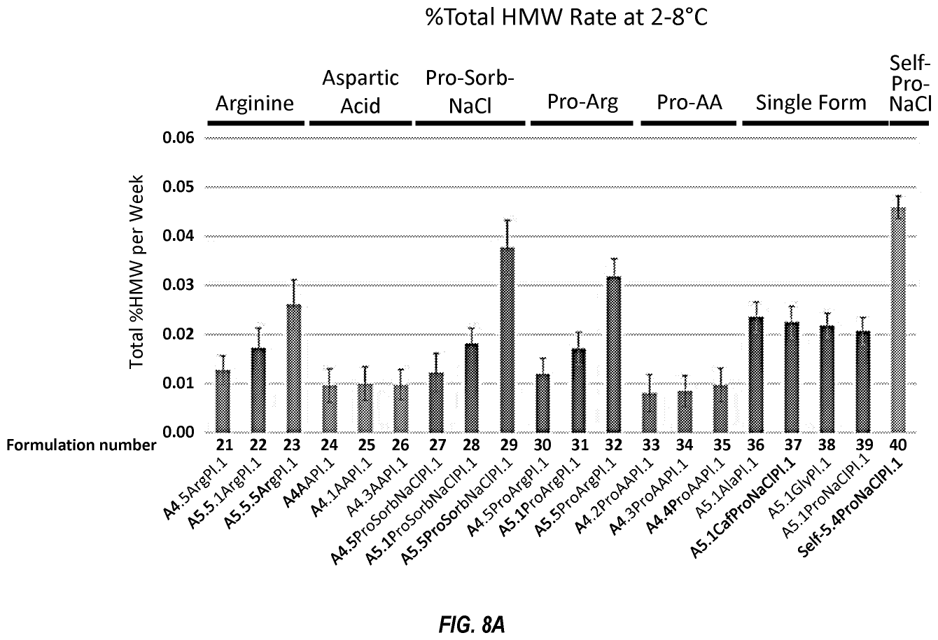

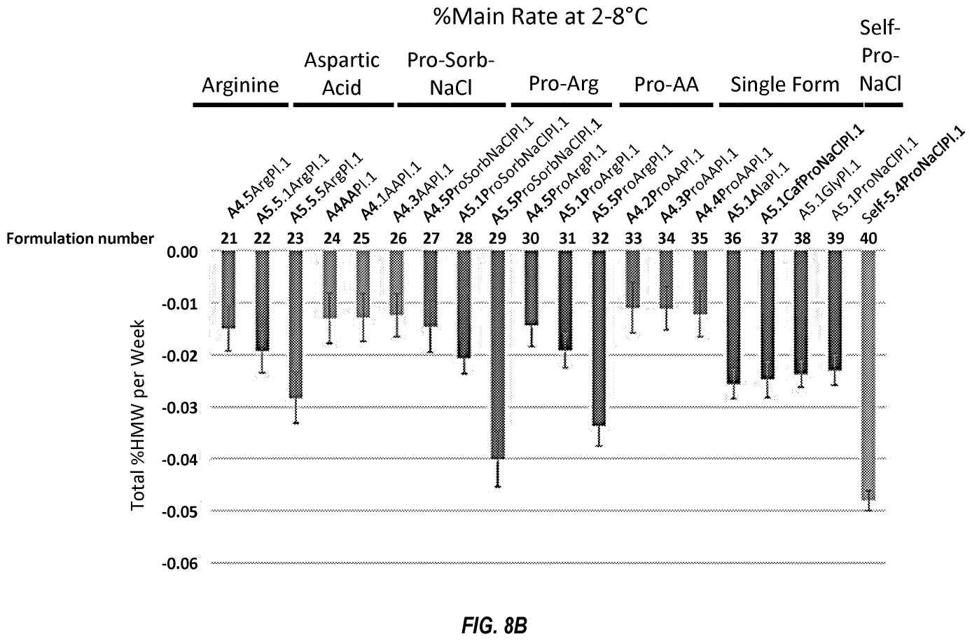

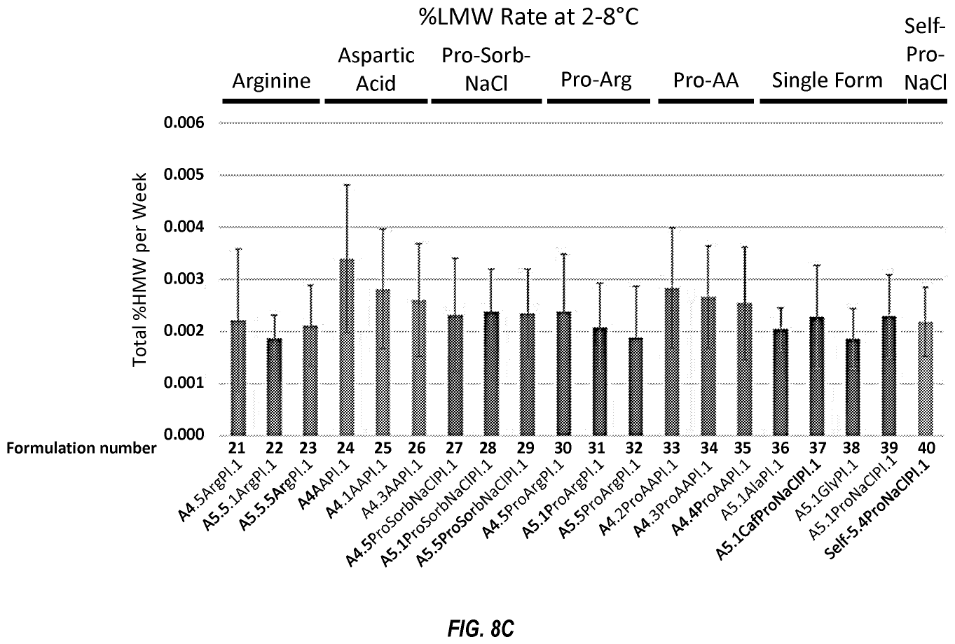

In the long term-stability study at 2-8° C., there was a pH-dependent impact on the rate of TACI-Fc form increase or loss in the formulation, as shown in FIG. 8A (% total HMW), FIG. 8B (% main rate), and FIG. 8C (% LMW). The results show that, upon storage at 2-8° C., generally, the greatest contributor to the reduction in the % main change rate (FIG. 8B) was the increase of the HMW species (FIG. 8A) compared to low molecular weight (LMW) species (FIG. 8C). Formulations 21-23, which contain the same formulation buffer prepared at different pH, had different changes in the percent main rate, and the differences were pH-dependent. Formulation 21, which was prepared at the lowest pH of 4.5, showed a lower rate change (decrease) in main percentage compared to the same formulation prepared at pH 5.1 and 5.5 (Formulations 22 and 23, respectively). For Formulations 21-23, preparation at the higher pH resulted in increased formation of the non-main HMW species. Similar results were shown for Formulations 30-32 (10 mM sodium acetate, 1.5% L-proline w/v, 1.55% L-arginine hydrochloride, 0.1% poloxamer 188 w/v at pH 4.5, 5.1 and 5.5, respectively). The impact of pH on the TACI-Fc species in the formulation depended on the makeup of the formulation; pH did not always impact the TACI-Fc species formation. For example, formulations ranging from pH 4 to 4.3 containing aspartic acid (Formulations 24-26) did not show a significant difference in change of % Main. These formulations were comparable to the formulations at pH 4.5 not containing aspartic acid. This may suggest that the beneficial impact of lowering the pH is minimal after an approximate pH of 4.5. At pH 5.1, the best performing formulation contained a charged excipient.

Results for the long-term stability study at 30° C. are shown in FIG. 9A (% total HMW), FIG. 9B (% main rate), and FIG. 9C (% LMW). In long-term stability studies with storage at 30° C., there were larger differences between the formulations at different pHs and using different excipients. For most formulations, the greatest contributor to the reduction in % Main species (FIG. 9B) was the increase in the % HMW (FIG. 9A). Stability at 30° C. for formulations at pH 5.1 showed that the proline-sorbitol-NaCl, alanine, caffeine-proline-sodium chloride, glycine and the selected proline-sodium chloride control from Example 2 were the worst performers. The formulations containing proline-sorbitol-sodium chloride exhibited a strong pH dependence on stability, with pH 5.1 and 5.5 having some of the highest rates of % Main degradation. Thus these formulations were determined not to be desirable due to the high pH dependence on stability. The highest stability at 30° C. was observed for formulations containing arginine HCl, aspartic acid, proline-arginine HCl, and proline-aspartic acid formulations. For the arginine HCl and arginine HCl-proline formulations, the lower the pH, the better the stability. For example, the arginine HCl (Formulations 21-23) and arginine HCl-proline formulations (Formulations 30-32) showed a pH-dependent impact on the rate change of % main, and the rate of formation of higher order TACI-Fc species (e.g., HMW). Of these formulations, those with lower pHs (e.g., Formulation 21 and 30) displayed better stability.

FIG. 9D shows the % HMW per week and % Main loss for formulations 21 and 39 from FIGS. 9A and 9B. Formulation 39 has the same formulation buffer components as Formulation 2, which showed increased TACI-Fc stability in Example 1, with a pH of 5.1 compared to Formulation 2 at pH 5.06. As depicted in FIG. 9D, Formulation 21, which is formulated at a pH of 4.5, showed much lower formation of HMW TACI-Fc species and lower loss of % Main compared to Formulation 39.

A further comparison of Formulation 21 (160 mg/mL TACI-Fc, 10 mM sodium acetate, 3.1% L-arginine hydrochloride w/v, 0.1% poloxamer 188 w/v, pH 4.5) and Formulation 39 (60 mg/mL TACI-Fc, 10 mM sodium acetate, 2% L-proline w/v, 50 mM sodium chloride, 0.1% poloxamer 188, pH 5.1) to Formulation 5 (160 mg/mL TACI-Fc, 10 mM sodium acetate, 3% L-proline w/v, 0.1% poloxamer 188 w/v, pH 5.21) and Formulation 2 (160 mg/mL TACI-Fc, 10 mM sodium acetate, 2% L-proline w/v, 50 mM sodium chloride, 0.1% poloxamer 188 w/v, pH 5.06) is shown in Table E12.

TABLE E12
% total HMW and % main species for four different formulations of 160 mg/mL TACI-Fc

| | Averaged % Total HMW at 2-8° C. | | | | | | Averaged % Main at 2-8° C. | | | | | |
|---|---|---|---|---|---|---|---|---|---|---|---|---|
| | Time (Weeks) | | | | Rate | Standard | Time (Weeks) | | | | Rate | Standard |
| Form. | 0 | 2 | 4 | 8 | (%/week) | Error | 0 | 2 | 4 | 8 | (%/week) | Error |
| 21 | 1.0 | 1.1 | 1.1 | 1.1 | 0.01 | 0.00 | 99.0 | 98.9 | 98.9 | 98.8 | −0.01 | 0.00 |
| 39 | 1.1 | 1.2 | 1.2 | 1.3 | 0.02 | 0.00 | 98.9 | 98.8 | 98.7 | 98.7 | −0.02 | 0.00 |
| 5 | 1.2 | 1.3 | 1.3 | 1.5 | 0.03 | 0.00 | 98.8 | 98.7 | 98.7 | 98.5 | −0.03 | 0.00 |
| 2 | 1.2 | 1.2 | 1.2 | 1.3 | 0.02 | 0.00 | 98.8 | 98.8 | 98.8 | 98.6 | −0.03 | 0.00 |

In contrast, for formulations containing only aspartic acid, such as Formulations 24-26, lower pHs were less stable, where there was greater loss of % main at the lower pH compared to the higher pH. These aspartic acid containing formulations at a pH close to 4 also had the greatest increase in % LMW (FIG. 9C). Examining the stability at both temperatures, the formulations of arginine HCl and arginine HCl-proline were the most stable, with the lowest pH of 4.5 being most stable, then pH 5.1, and pH 5.5. In particular, the results showed that Formulation 21, containing 3.1% L-arginine hydrochloride (w/v) and 10 mM sodium acetate, formulated at pH 4.5 showed stabilizing effects, as assessed by SEC.

### 3. Background Membrane Imaging (BMI)

Background membrane imaging (BMI) was used to measure subvisible particles for all samples after storage for 8 weeks at 2-8° C. and 30° C. The measurement was made substantially as detailed in Example 2, except the time zero samples were allowed to “rest” for 24 hours and 3 days at 2-8° C. post-plating before BMI measurement.

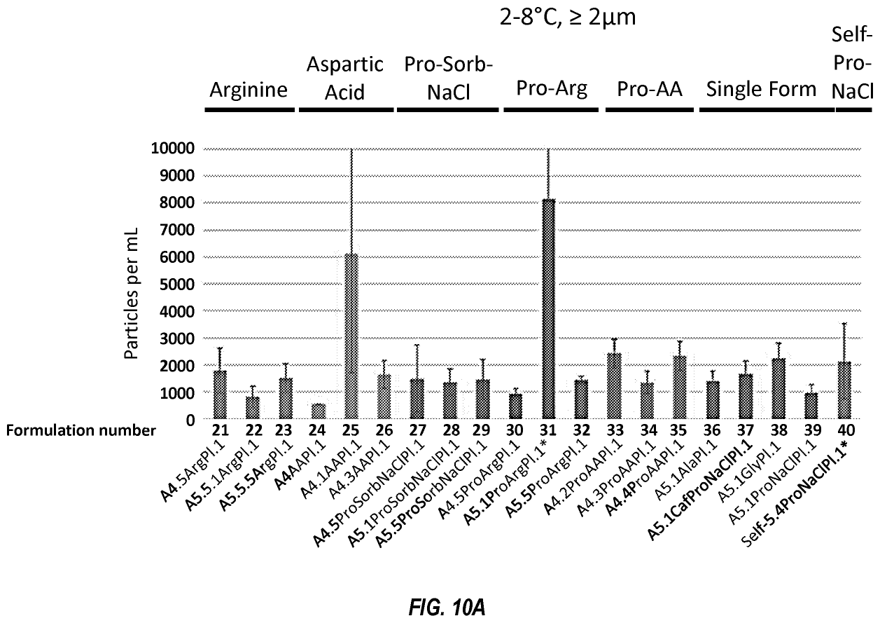

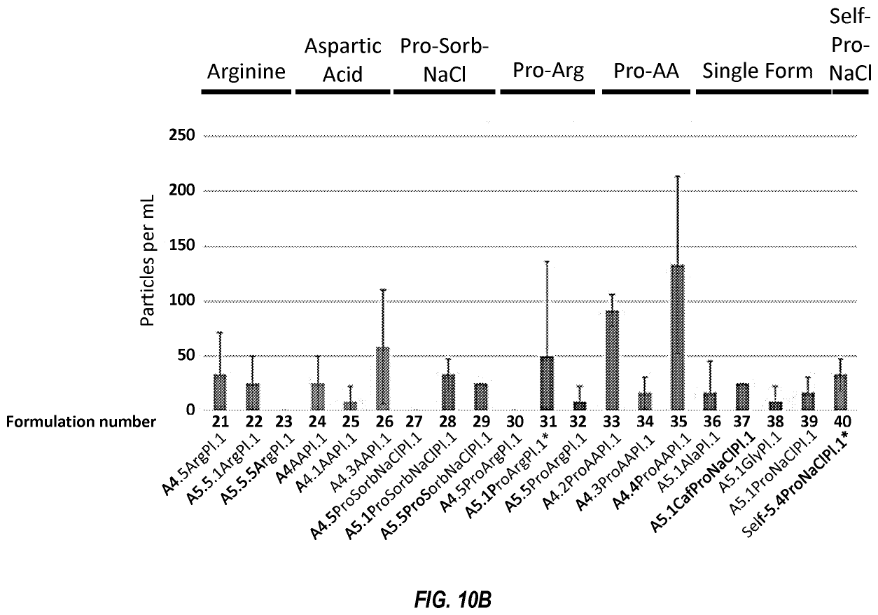

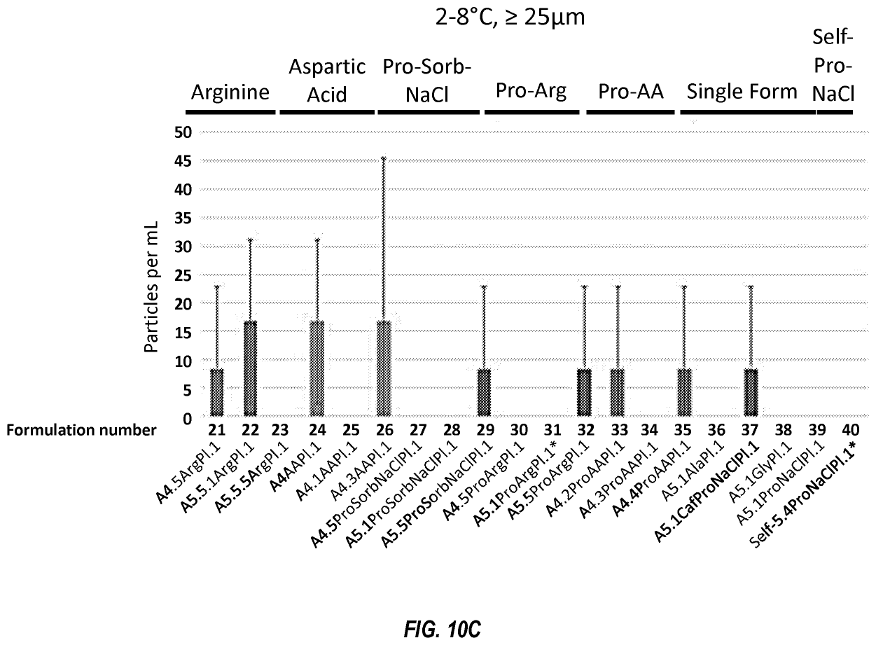

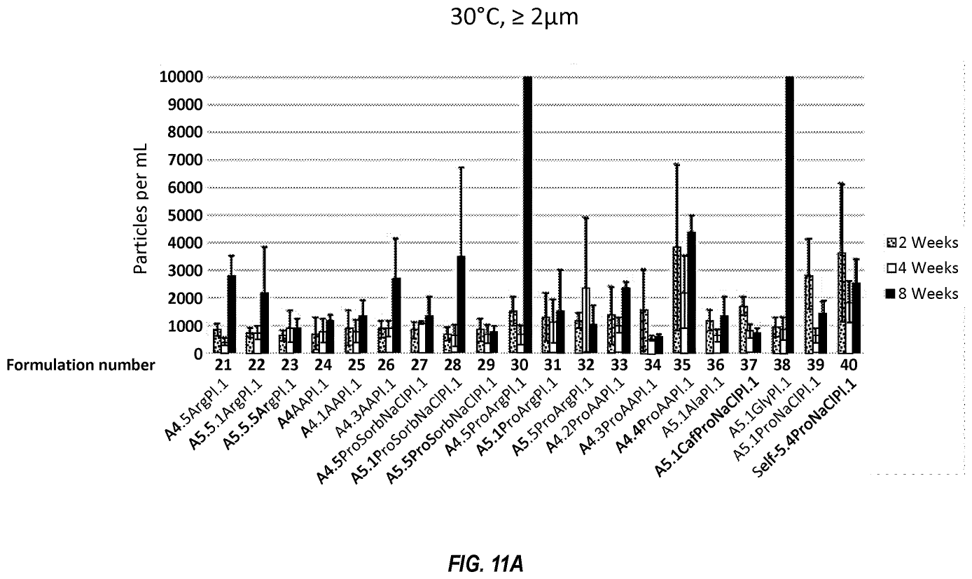

The number of particles that were ≥2 μm, ≥10 μm or ≥25 μm per mL, were counted. In general, the total particle counts increased slightly over time for particles ≥2 μm, however, it was not determined to be significant based on the average counts and associated standard deviations. To help differentiate stability between formulations after long-term storage at 2-8° C., the final time point of eight weeks was graphed. A noticeable difference between the formulations was not observed, with particles counts being low at ≥10 μm and ≥25 μm, as shown in FIGS. 10A-10C.

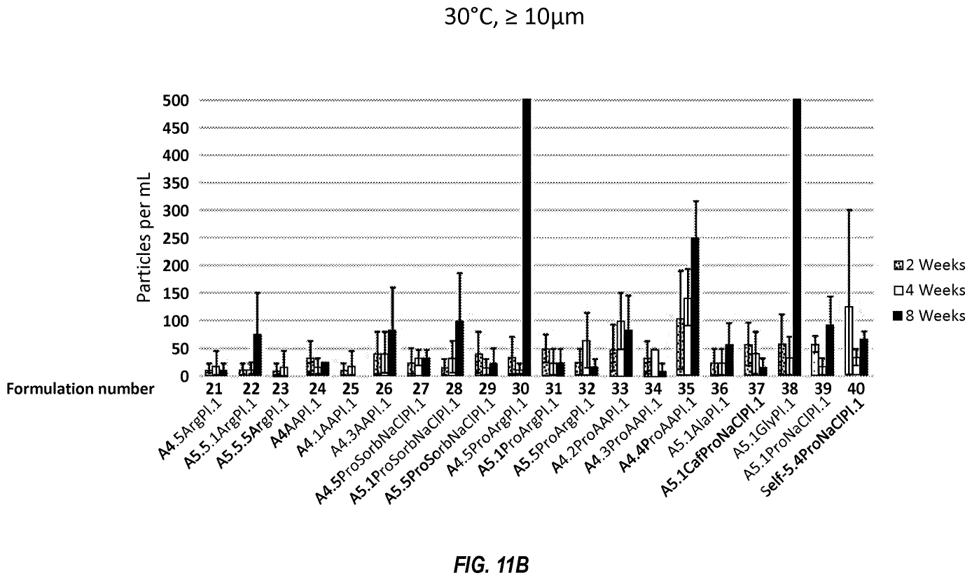

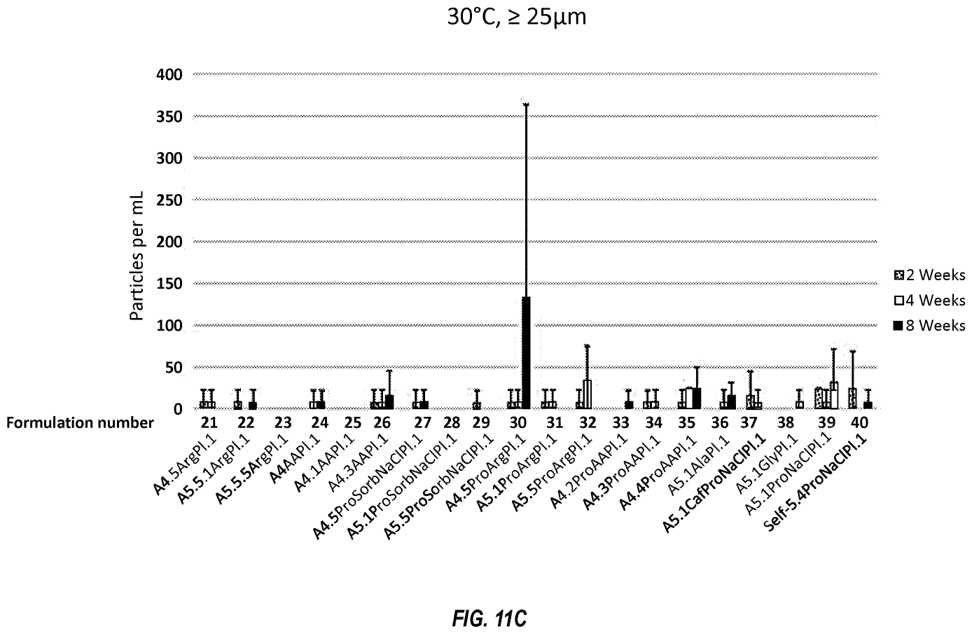

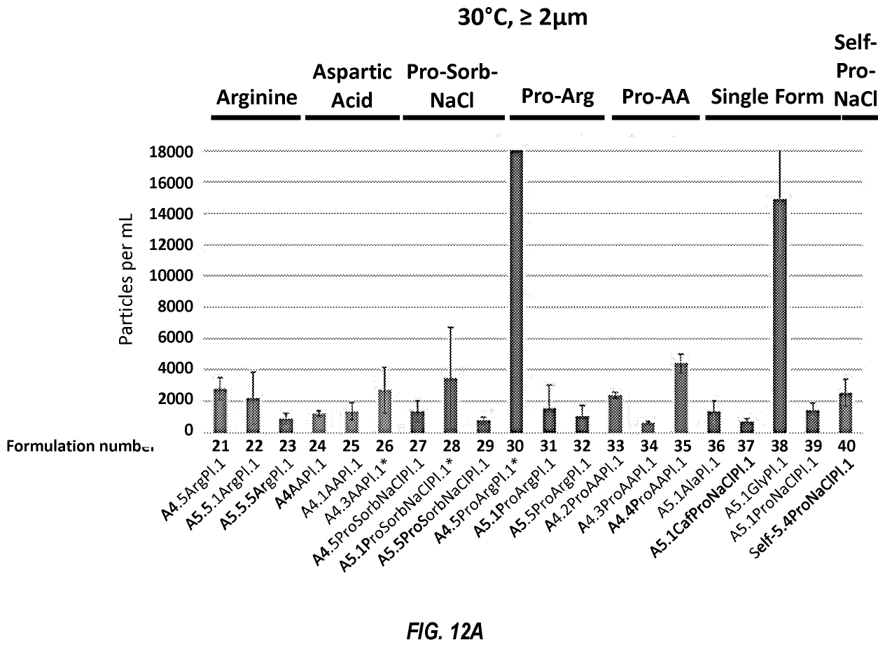

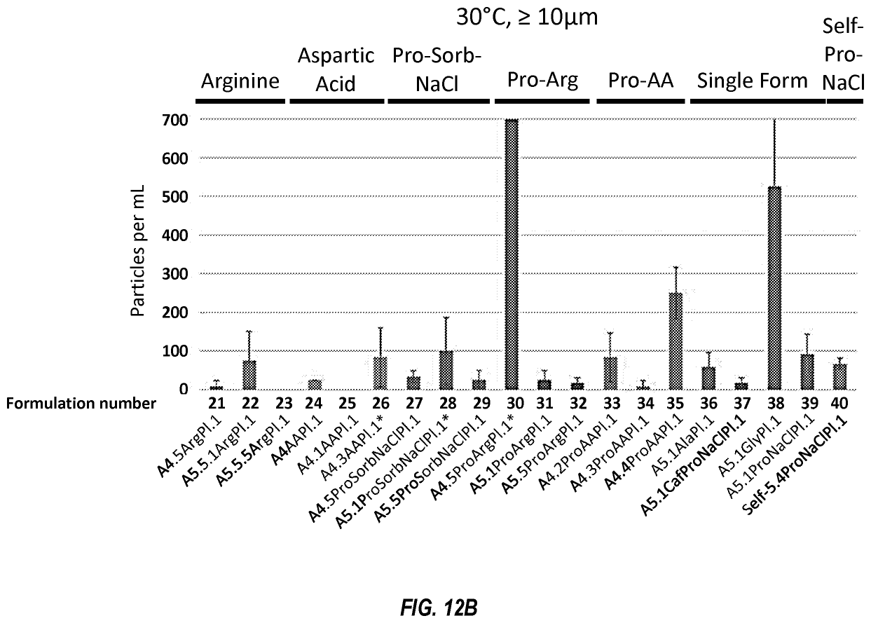

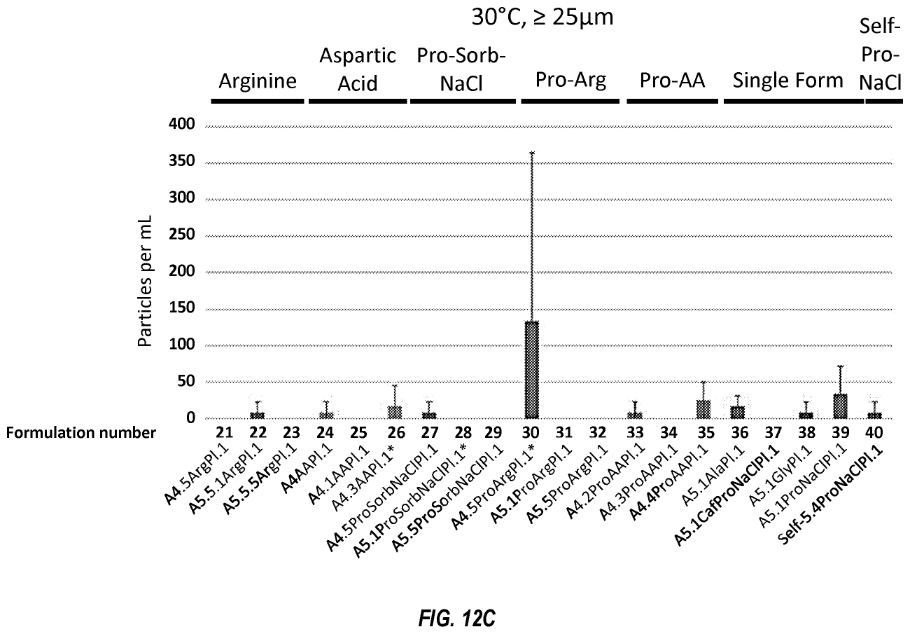

FIGS. 11A-11C show the particle counts by BMI over time at 30° C., for particles ≥2 μm, ≥10 μm, or ≥25 μm, respectively. FIGS. 12A-12C show the particle counts by BMI at the end point of the study, 8 weeks. In general, the total particle counts within a formulation were comparable over time for particles ≥2 μm, based on the overlapping error bars. Examining across formulations, a strong difference was not measured, with particles counts being low at ≥10 μm and ≥25 μm.

### 4. Qualitative Analysis

The particle images of the BMI analysis from individual wells were also qualitatively examined, to see if any particle trends could be observed based on the particle images. After two weeks at 2-8° C., a difference was not observed between the formulations for particle types. After four weeks, no obvious particle trends were noted at 2-8° C. At 30° C. at four weeks, the results show that formulations with acetate-proline-aspartic acid showed large, amorphous particles, regardless of the formulation pH. These particles were not present in other formulations such as acetate-aspartic acid. After 8 weeks at 2-8° C., formulations containing aspartic acid had “out of trend” particles. For acetate-aspartic acid, the particles characteristics were dependent on pH. At 30° C., different particle types were observed for all formulations except for the caffeine-containing formulation.

### 5. Summary

By SEC, the most stabilizing formulations at both 2-8° C. and 30° C. were the arginine HCl and arginine HCl-proline formulations, with the lowest pH studied of pH 4.5 being the most stabilizing. While the aspartic acid containing formulations showed comparable stability to the arginine HCl formulations at 2-8° C., the lower pH of these formulations ranging from pH 4 to 4.3 showed negative effects at 30° C. The stability was pH-dependent, with a pH 4 performing worse than pH 4.3. More fragmentation was measured by SEC for these formulations as the pH was lowered. Comparing the arginine HCl and arginine HCl-proline based formulations to the selected formulation from Example 2 (proline-NaCl), the formulations at pH 5.1 were performing comparably at 2-8° C., however, at 30° C., the arginine HCl formulations were almost five-fold lower than the proline-NaCl formulation.

By particle counts measured by BMI, a significant difference between formulations was not observed at 2-8° C. A strong difference was not measured between formulations at 30° C. over time.

Based on the results from SEC and BMI, the formulations containing arginine HCl or arginine HCl-Proline at pH 4.5 were among the most improved formulations that exhibited favorable attributes for manufacturability and syringability, including the ability to maintain an active form (homodimer) after extended storage at low and high temperatures, and a viscosity within the acceptable range for administration of liquid pharmaceutical formulations.

### Example 4

### Assessing the Viscosity of Formulations Containing Different TACI-Fc Protein Concentrations

The results in Examples 2 and 3, above, indicate that pH can impact the stability of TACI-Fc. The results also show that TACI-Fc stability improved in the presence of charged excipients. Next, formulations from Examples 2 and 3 that showed high stability were examined to further assess if pH influences viscosity. Formulations 41-47, which are described in Table E13, were assessed. Formulation 21, containing 10 mM sodium acetate, 3.1% L-arginine hydrochloride at pH 4.5, was shown in Example 3 to have high stability; Formulations 41 and 42 contain the same formulation components as Formulation 21 except they are formulated at pH 4.1 and 4.6, respectively. Formulation 43 corresponds to Formulation 14 from Example 2, which had high stability, but was formulated in this example at a lower pH as was assessed in Example 3. Formulations 44 and 45 were examined here as alternative formulations to Formulations 34 and 35, which performed well in Example 3. These have a very low pH buffered range. Formulation 46 was tested by formulating with caffeine as caffeine has been reported to reduce viscosity of pharmaceutical formulations.

A summary of the tested formulations is set forth in Table E13.

TABLE E13
Formulations

| | Formulation Description | | |
|---|---|---|---|
| Formulation | TACI-Fc fusion protein at different | pH Target at | |
| Number | concentrations + listed excipients | 160 mg/mL | Acronym |
| 41 and 42 | 10 mM sodium acetate, 3.1% L-arginine | 4.1 and 4.6 | A41Arg and |
| | hydrochloride w/v | | A46Arg |
| 43 | 10 mM sodium acetate, 2% proline w/v, 50 mM NaCl | 4.6 | A46ProNaCl |
| 44 | 10 mM sodium acetate, 2.7% proline w/v, 0.4% | 4.3 | A43ProAA |
| | Aspartic Acid w/v | | |
| 45 | 10 mM sodium acetate, 1.7% Proline w/v, 0.4% | 3.9 | A39ProAANaCl |
| | Aspartic Acid w/v, 50 mM NaCl | | |
| 46 and 47 | 10 mM sodium acetate, 2.8% Arginine HCl w/v, 28 | 4.1 and 4.6 | A41CaffArg and |
| | mM caffeine | | A46CaffArg |

TACI-Fc material described in Example 1 was prepared to 10 mg/mL and kept frozen at −70° C. until needed. Material was buffer exchanged using 15 mL, 10 kD Centricon® filters (Sigma, UFC901024D) for all formulations. After buffer exchange, material was over concentrated and collected. Then, placebo buffer was used to create a dilution curve for the viscosity curve. For the placebo buffers containing aspartic acid (Formulation 44 and 45), 0.1 M sodium hydroxide was used to hit the pH target of the placebo.

For Formulations 46 and 47, SEC was used for quantifying the protein concentration; the presence of caffeine confounded the SoloVPE measurements. TACI-Fc in 10 mM sodium acetate, 3.1% w/v arginine HCl at 160 mg/mL was used to create a standard curve by injecting different volumes and then measuring the area under the curve. From this standard curve, the caffeine containing samples could then be quantified for protein concentration.

The viscosity curves of the formulations described in Table E13 were measured. Each protein concentration was measured for viscosity across multiple shear rates from 500 to 1000 1/s at 20° C. and averaged.

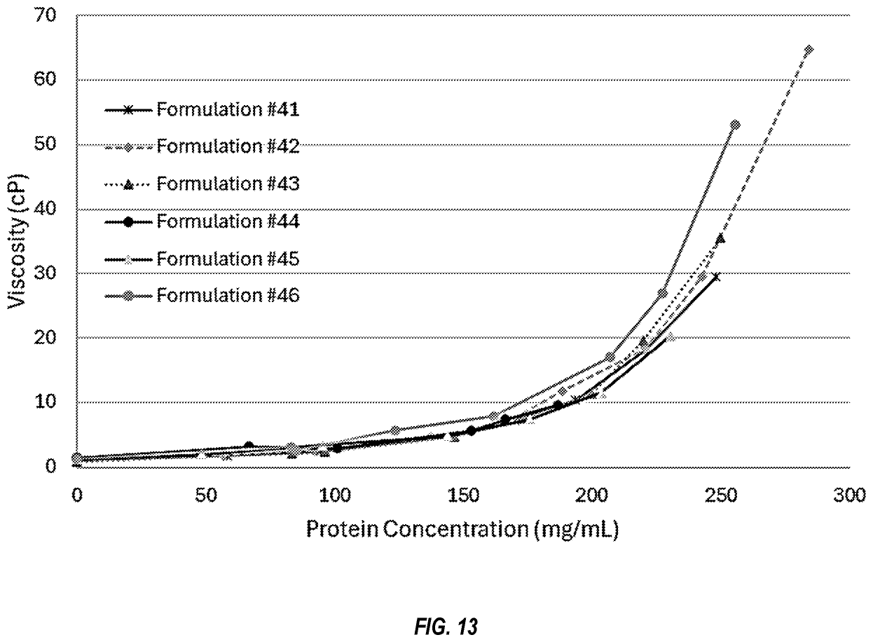

The results are set forth in FIG. 13, which shows the comparison of the viscosity curves for all formulations.

### Summary

The results show that there was not a strong difference between the different viscosity curves of TACI-Fc in the studied formulations. A pH of 4.1 or 4.6 resulted in comparable viscosity curves in acetate-arginine HCl which was in turn comparable to formulation 43 (A46ProNaCl). The use of aspartic acid limited the highest concentration that could be reached (187 mg/mL) in formulation 44 (A43ProAA). When sodium chloride was added to this formulation (formulation 45, A39ProAANaCl), a higher concentration could be reached, but did not cause a strong change from formulation 42 (A46Arg). The caffeine-based formulation also did not cause a strong difference from formulation 42 (A46Arg). These results show that TACI-Fc can be formulated at a concentration of 175 mg/mL without adversely impacting viscosity in these tested formulations.

### Example 5

### Assessing the Surfactant on the Stability of a TACI-Fc Fusion Protein

Studies were carried out to assess the impact of the type of surfactant in a formulation buffer on the long-term stability of a TACI-Fc fusion protein by monitoring the stability and other features of the TACI-Fc fusion protein homodimer for up to 24 months. In these studies, formulations containing TACI-Fc fusion protein in a buffer containing 10 mM sodium acetate, 3% proline w/v formulated at pH 5.2 with either 0.015% polysorbate 80 (PS80) w/v or 0.1% Poloxamer 188 (P188) w/v were prepared. Each formulation also was designed to contain 100 mg/mL TACI-Fc. The stability of the TACI-Fc fusion protein in the different buffers after prolonged storage at either −70° C., −20° C., 2-8° C., 25° C., or 40° C., was assessed.

A summary of the tested formulations is set forth in Table E14.

TABLE E14
Formulations

| Formulation Description TACI-Fc fusion protein | pH Target at |
|---|---|
| at 100 mg/mL + listed excipients | 100 mg/mL |
| 10 mM sodium acetate, 3% proline w/v, 0.015% | 5.2 |
| polysorbate 80 (PS80) w/v | |
| 10 mM sodium acetate, 3% proline w/v, 0.1% | 5.2 |
| Poloxamer 188 (P188) w/v | |

Formulations were prepared as detailed in Example 2.A where the TACI-Fc fusion protein was concentrated using cassette filters. Formulations were filled to a volume of 1.4 mL in a glass ISO2R adptiQ vial (from Schott), with 1 mL extractable volume, with a 12 mm chlorobutyl stopper (from West, product number 19700004).

### A. Characterization of the TACI-Fc in Different Formulations

The properties of the TACI-Fc in the various formulations were assessed to determine the impact of the formulation components with different types of surfactant on TACI-Fc long-term stability. Using the assays listed in Table E15, stability of TACI-Fc formulated in different formulation buffers containing the different surfactants PS80 or P188 in the different amounts (0.015% and 0.1% w/v, respectively) was assessed. The 2 formulations were assessed for stability at −70° C., −20° C., 2-8° C., 25° C., or 40° C. for up to 24 months according to the schedule shown in Table E16. Additionally, the number of subvisible particles was measured and the visual properties of the formulations were assessed at time zero and at various times points for both formulations.

Stability of the TACI-Fc homodimer in the two formulation buffers was assessed after storage at varying temperatures using the assays set forth in Table E15, below:

TABLE E15
Assays for Assessing TACI-Fc Stability

| Designator | Assay |
|---|---|
| A | SEC, rCE-SDS, icIEF (icIEF), and/or potency against |
| | APRIL and BAFF |
| B | Color, clarity, visible particles, subvisible particles |
| | (HIAC) |
| C | Concentration |
| D | pH, retains for MAM |
| E | Osmolarity |

A summary of the stability assays, storage temperature, and storage times assessed in this study is set forth in Table E16.

TABLE E16
Conditions Tested and Assays for Each Condition and Timepoint

| | −70° C. | −20° C. | 2-8° C. | 25° C. | 40° C. |
|---|---|---|---|---|---|
| 2 weeks | AB | AB | AB | AB | AB |
| 4 weeks | AB | AB | AB | AB | AB |
| 8 weeks | AB | AB | AB | AB | AB |
| 3 months | AB | AB | AB | AB | ABCD |
| 6 months | ABC | ABC | ABC | ABCD | |
| 12 months | ABC | ABC | ABC | | |
| 18 months | ABC | ABC | ABC | | |
| 24 months | ABCD | ABCD | ABCD | | |

### 1. Size Exclusion Chromatography (SEC)

SEC was used to assess stability and aggregation, and the forms of the TACI-Fc in the two formulation buffers containing the different surfactants (PS80 or P188) in the different amounts (0.015% and 0.1%, respectively) after storage for up to 24 months at −70° C., −20° C., 2-8° C., 25° C., and 40° C. The change in 0% main (TACI-Fc homodimer) per week was calculated after incubation at −70° C., −20° C. and 5° C. for 24 months; at 25° C. for 6 months; or at 40° C. for 3 months. The SEC assessments were conducted as described in Example 2, and the loss of the ‘main’ species of TACI-Fc was assessed over time.

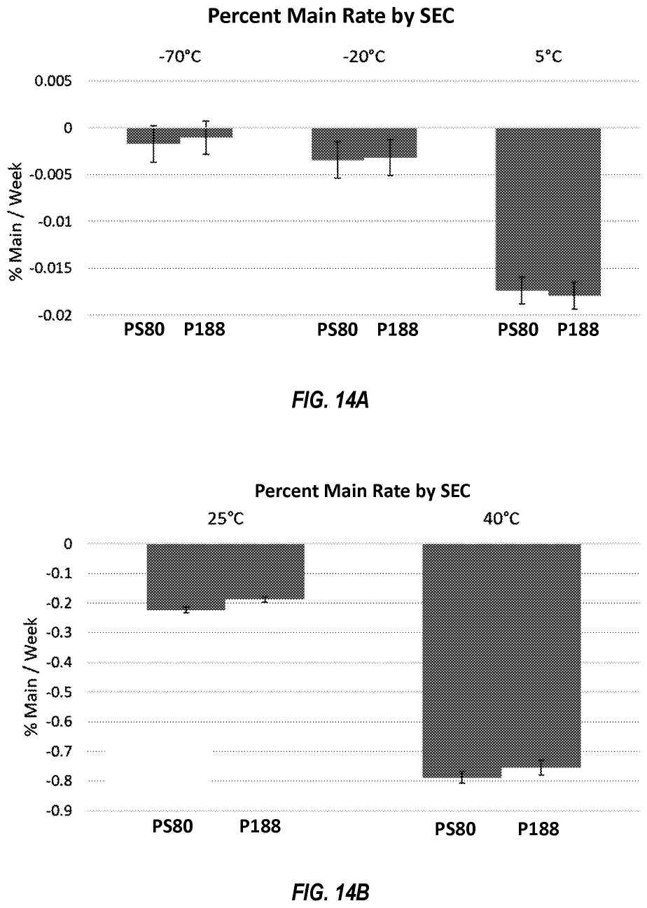

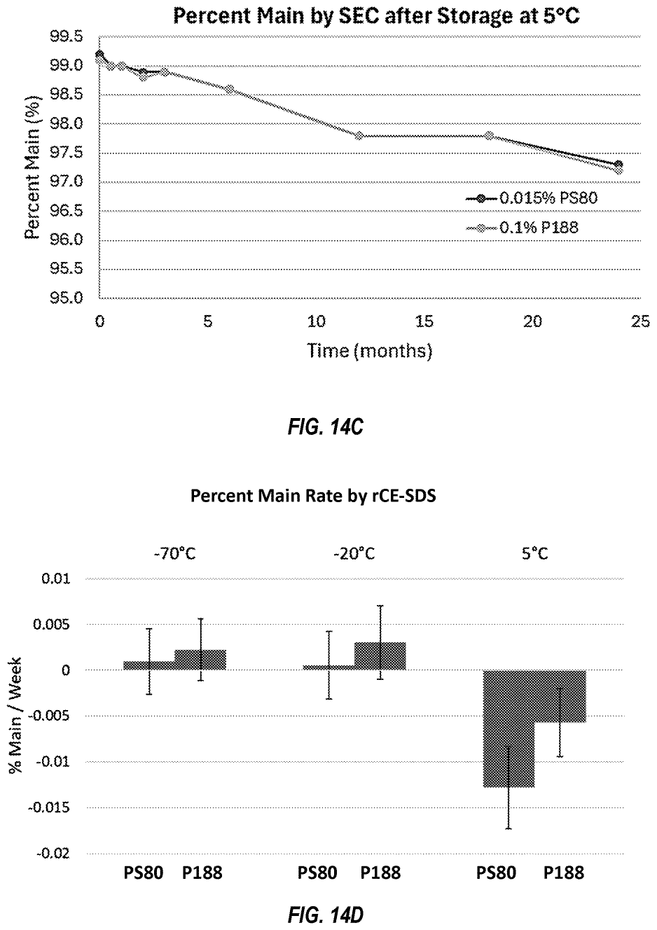

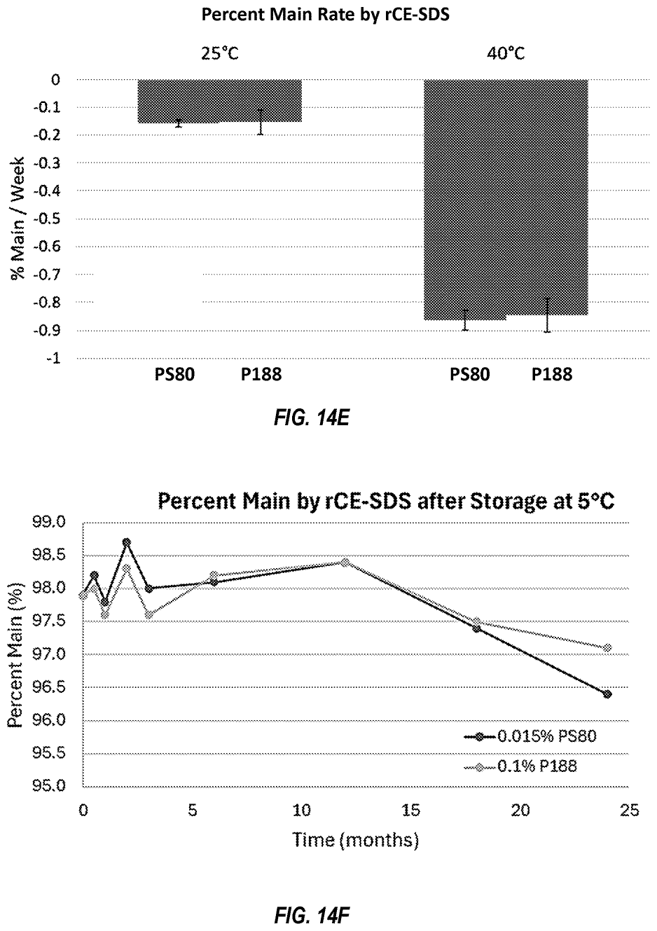

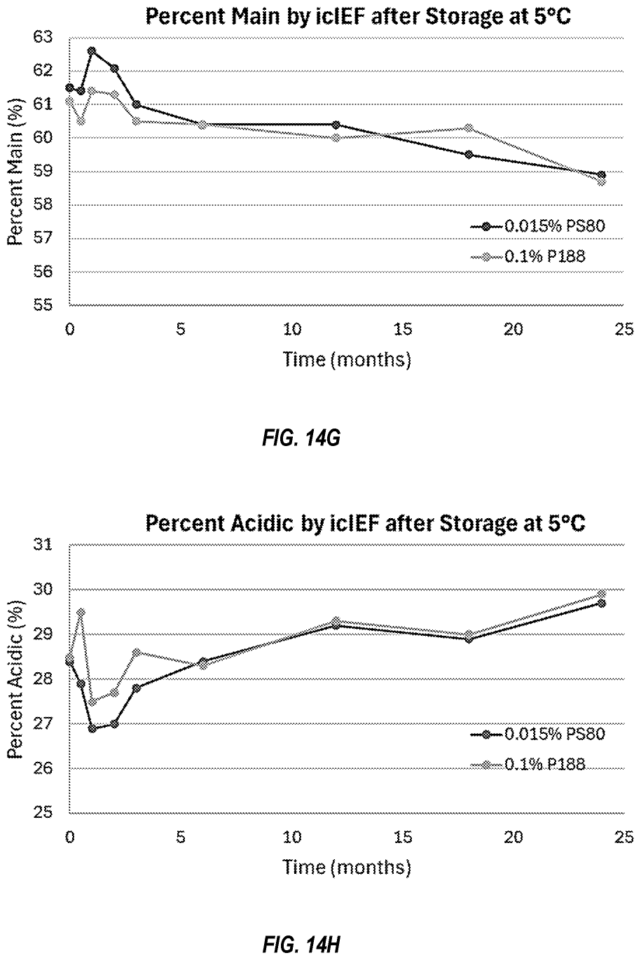

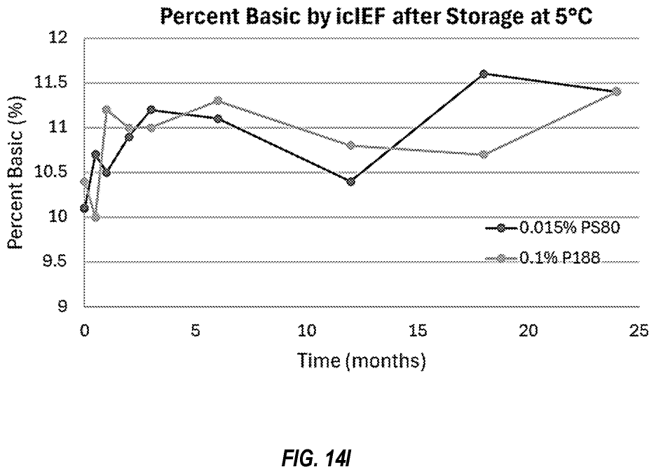

The results of analysis by SEC show that the percent of the TACI-Fc that is in the ‘main’ species (homodimeric form) decreased with increasing temperature after long term storage. The highest decrease in percent main was after incubation at 40° C. and the lowest decrease in percent main was after incubation at −70° C., as shown in FIGS. 14A-C. As shown in FIG. 14C, the SEC results show that the percent main decreases over time after storage for 24 months at 5° C., with the greatest decrease after incubation at 24 months of the TACI-Fc in both formulation buffers. The results also show that TACI-Fc stability, as assessed by the loss of the percent main, was not impacted by the surfactant choice in the buffer containing 10 mM sodium acetate, 3% proline w/v formulated at pH 5.2 at 2-8° C. The loss of percent main after prolonged incubation at different temperatures in this formulation buffer containing 0.015% PS80 or 0.1% Poloxamer 188 (P188) was similar as assessed by SEC.

### 2. Reduced Capillary Electrophoresis Sodium Dodecyl Sulfate (rCE-SDS)

rCE-SDS was used to assess stability and clipping of the TACI-Fc in the two formulation buffers containing the different surfactants (PS80 or P188) in the different amounts (0.015% and 0.1%, respectively) after storage for up to 24 months at −70° C., −20° C., 2-8° C., 25° C., and 40° C. The change in % main (TACI-Fc homodimer) per week was calculated after incubation at −70° C., −20° C. and 5° C. for 24 months; at 25° C. for 6 months; or at 40° C. for 3 months. Briefly, samples were prepared with a sample buffer containing a reducing agent and SDS, then heated to denature and reduce the disulfide bonds of the TACI-Fc fusion protein in the sample. The samples were then applied to a capillary, voltage was applied for electrophoretic migration, and analytes were detected at 220 nm as they migrate off the capillary.

The results of analysis of the TACI-Fc forms after prolonged incubation at different temperatures by rCE-SDS were similar to the SEC results. After long term storage, the percent of the TACI-Fc that is in the ‘main’ species (homodimeric form) decreased with increasing temperature in both formulations containing the different amounts and type of surfactant. The highest decrease in percent main was after incubation at 40° C. and the lowest decrease in percent main was after incubation at −70° C. and −20° C. in both formulations, as shown in FIGS. 14D-F. 14. The rCE-SDS results show that the percent main decreases over time after storage for 24 months at 5° C., with the greatest decrease after incubation at 24 months of the TACI-Fc in both formulation buffers. These results indicate that the TACI-Fc main species has comparable stability in formulations containing 10 mM sodium acetate, 3% proline w/v formulated at pH 5.2 and either 0.015% PS80 or 0.1% P188 after prolonged incubation at 2-8° C., when assessed by rCE-SDS.

### 3. Imaged Capillary Isoelectric Focusing (icIEF)

Imaged capillary isoelectric focusing (icIEF) was used to assess the TACI-Fc forms and the charge heterogeneity of the TACI-Fc fusion protein prepared in a buffer containing either 0.015% [w/v] PS80 or 0.1% [w/v]P188 and stored at different temperatures and times as discussed above. Test samples were diluted, then mixed with sample buffer containing methyl cellulose, urea, ampholytes, arginine, pI markers, and water. Prepared samples were loaded into the sample wells of a 96-well plate or sample vials, and sample analysis was performed using an icIEF instrument, which resolves acidic variants (peak[s] focusing at a pI lower than the main peak), main peak, and basic variants (peak[s] focusing at a pI higher than the main peak).

The results of analysis of the TACI-Fc forms after prolonged incubation at different temperatures by icIEF show a decrease in the percent main over time after incubation at 5° C. for 24 months for both formulations, with a similar decrease for each formulation, as shown in FIG. 14G. The icIEF results also show that there was not a significant difference between formulation buffers in the percent basic (% basic) or percent acidic (% acidic) variants of the TACI-Fc after storage for 24 months at 5° C., as shown in FIGS. 14H and 14I. These results indicate that the TACI-Fc homodimer has comparable stability in formulations containing 10 mM sodium acetate, 3% proline w/v formulated at pH 5.2 and either 0.015% PS80 or 0.1% P188 after prolonged incubation, when assessed by icIEF.

### 4. Subvisible Particle Assessment

Various types of particulate impurities and protein aggregates can cause immune reactions after administration of therapeutic formulations and protein aggregation can reduce or eliminate the effectiveness of the administered protein therapeutic. The number of subvisible particles in formulations containing TACI-Fc protein was assessed by light obscuration.

A HIAC Liquid Particle Counter (Beckman Coulter) particle analyzer was used to measure subvisible particles for both samples after storage for 24 months at 5° C. and six months at 25° C. Assessments were conducted in accord with the manufacturer's instructions to count the subvisible particles in the samples. USP guidelines specify no more than 6,000 ≥10 μm particles per container. The cumulative number of particles ≥10 μm or ≥2 μm per container, was counted at the designated temperatures and time points in Table E16.

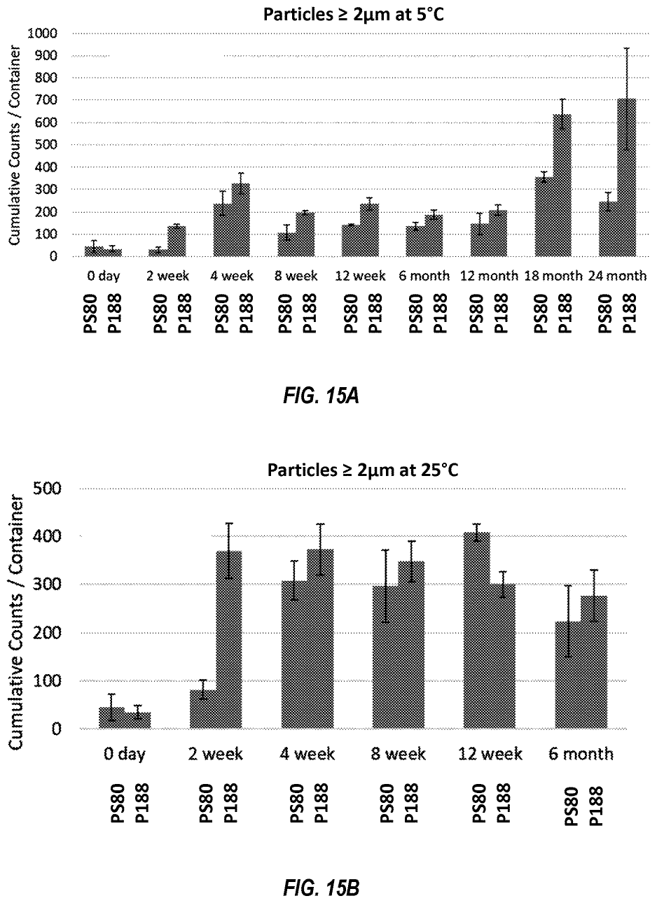

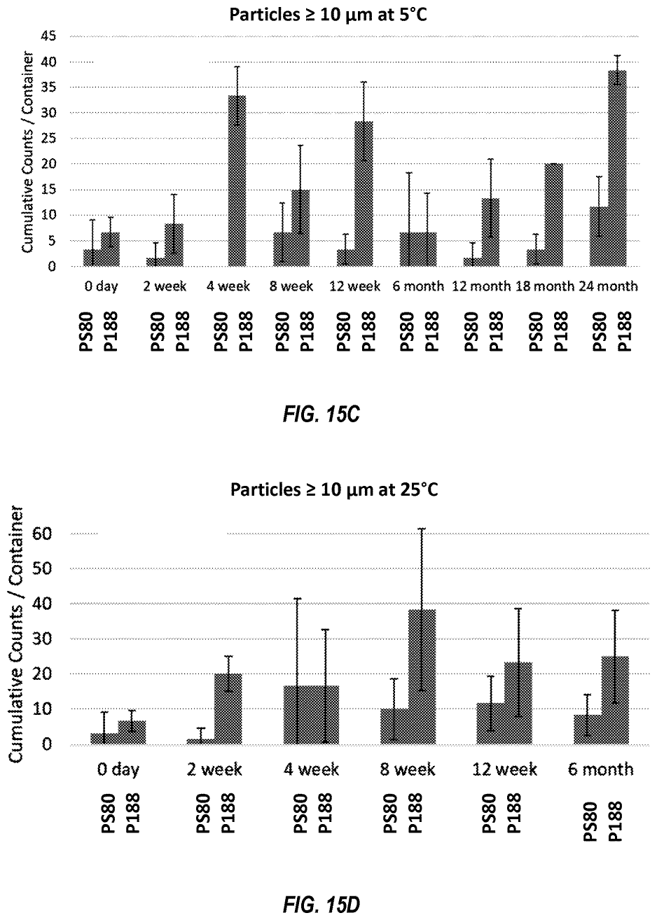

The TACI-Fc formulations containing 0.1% poloxamer 188 and 0.015% polysorbate 80 had comparable numbers of subvisible particles after 24-month storage at 5° C., and were within the variability of the assay. FIGS. 15A-D show the particle counts for ≥2 μm or ≥10 μm particles by light obscuration over 24-month incubation at 5° C. and 6 months at 25° C. The number of particles for both formulations were within USP guidelines at all timepoints and for both formulations.

### 5. Visual Inspection

The visual attributes of the various formulations were assessed to determine if the type of surfactant and prolonged storage had a negative impact on these attributes. Variation in a formulation's color and/or clarity can indicate the presence of contaminations, impurities or degraded forms of the TACI-Fc protein. The two formulations were assessed by eye for color, clarity, and the presence of visible particles after storage at −70° C., −20° C., 2-8° C., 25° C., and 40° C. for up to 24 months.

The color of the prepared TACI-Fc formulations was visually inspected in alignment with European Pharmacopeia Chapter EP 2.2.2 with the following exceptions: instead of using test tubes for visual analysis, color is observed in the original sample container and compared to BY color standards at the same volume in the same container type. Observation is made against a white background under ambient light, viewing horizontally.

Assessment of the color of the formulations show a color of <BY5 after storage at 5° C. and below for both formulations at all time points; <BY4 at 25° C., 6 months for the formulation containing Poloxamer 188; <BY5 for the formulation containing PS80 at 25° C. after 6 months; and <BY4 at 40° C. at 1-3 months for both PS80 and Poloxamer 188 formulations. This color was within the acceptable range for therapeutic formulations.

The relative clarity of the prepared TACI-Fc formulations was visually inspected in alignment with European Pharmacopeia Chapter EP 2.2.1 with the following exceptions: instead of using test tubes, clarity is observed in the original sample container and compared to standards at the same volume in the same container type. Observation is made against a black background using a high intensity light booth, viewing horizontally. The clarity of the formulations with both surfactants were clear for up to 24 months at ≤2-8° C., and up to 3 months for storage at 40° C. and 6 months at 25° C., which were the longest timepoints tested.

The presence of visible particles in the prepared TACI-Fc formulations was assessed by visible inspection based on United States Pharmacopeia Chapter <790>. The results from assessing visible particles show that all formulations were essentially free of visible particles for all time points and temperatures studies. There was rarely a single fiber and/or particle observed.

Visual inspection of the formulations on stability containing either 0.015% PS80 w/v or 0.1% P188 w/v show that a formulation with either surfactant results in a clear and slightly colored formulation essentially free of visible particles. A summary of the visual inspection assessments is set forth in Table E17. These results show that the color, clarity and presence of visible particles was within the acceptable range for therapeutic formulations.

TABLE E17
Summary of Visual Inspection Results at the Experiment End Point

| | 10 mM sodium acetate, 3% proline | | | | | | | | | | | |
|---|---|---|---|---|---|---|---|---|---|---|---|---|
| | w/v, 0.015% polysorbate 80 (PS80) | | | | | | 10 mM sodium acetate, 3% proline | | | | | |
| | w/v at pH 5.2 with 100 mg/mL | | | | | | w/v, 0.1% Poloxamer 188 w/v at pH | | | | | |
| Formulation | TACI-Fc | | | | | | 5.2 with 100 mg/mL TACI-Fc | | | | | |
| Temperature | 5° | C. | 25° | C. | 40° | C. | 5° | C. | 25° | C. | 40° | C. |
| End Point | 24 | months | 6 | months | 3 | months | 24 | months | 6 | months | 3 | months |

| | | | | | | |
|---|---|---|---|---|---|---|
| Color | <BY5 | <BY5 | <BY4 | <BY5 | <BY4 | <BY4 |
| Clarity | Clear | Clear | Clear | Clear | Clear | Clear |
| Visible | EFVP | EFVP | EFVP | EFVP | EFVP | EFVP |
| Particles | | | | | | |

EFVP: essentially free of visible particulates
PFFP: practically free of visible particulates

### 4. TACI-Fc Anti-APRIL and Anti-BAFF Activity

TACI-Fc fusion proteins can bind to BAFF and APRIL ligands to neutralize their activity. Studies were conducted to assess whether the TACI-Fc in the formulations retained neutralizing activity for APRIL and BAFF after prolonged storage in the formulation buffers containing the different surfactants. The bioactivity of the TACI-Fc protein stored in formulation buffers containing either the PS80 or P188 surfactants at concentrations of 0.015% and 0.1% w/v, respectively, for up to 104 weeks (24 months) at −70° C., −20° C., 5° C., 25° C., and 40° C. for APRIL and BAFF was assessed. Activity of the TACI-Fc for APRIL and BAFF was assessed with binding ELISAs. Test samples were prepared and plated on assay plates adsorbed with APRIL or BAFF. Bound TACI-Fc was subsequently detected using a peroxidase conjugated Fc-specific goat anti-human IgG antibody. 3,3′,5,5′ TMB HRP substrate is added, which produces a chromogenic reaction. The enzymatic reaction was stopped using phosphoric acid. The reaction was quantified by measuring the absorbance of the reaction product at 450 nm which is proportional to the amount of the TACI-Fc fusion protein bound to APRIL or BAFF. The percent relative potency of the test sample was determined by comparison of the dose response curve mid-point value to that of the reference standard.

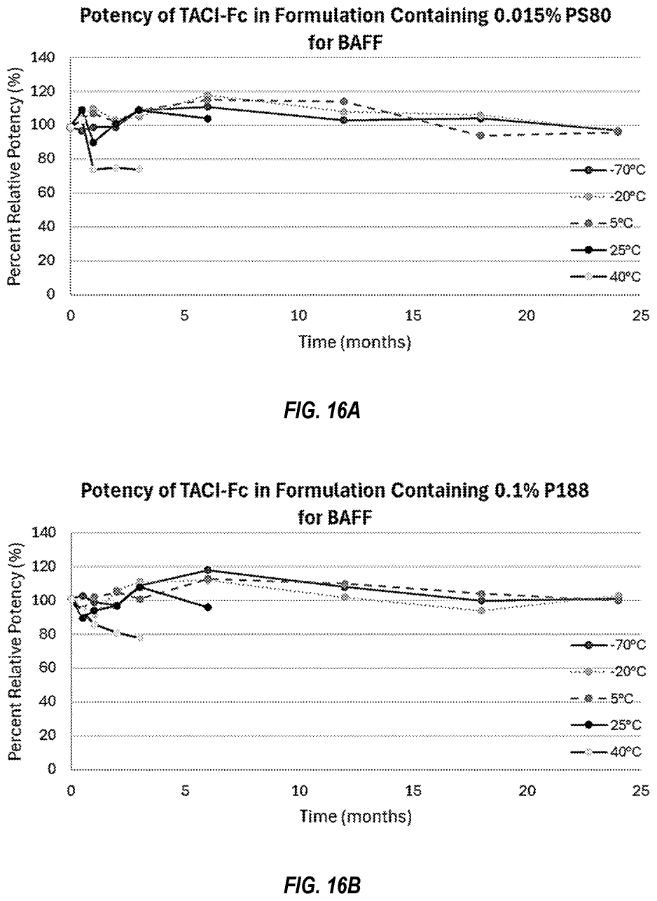

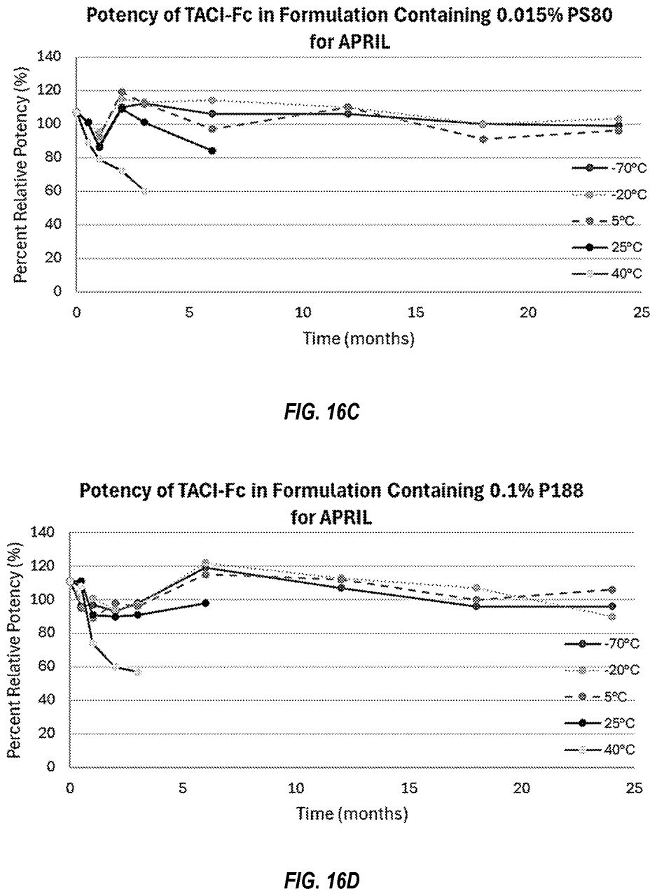

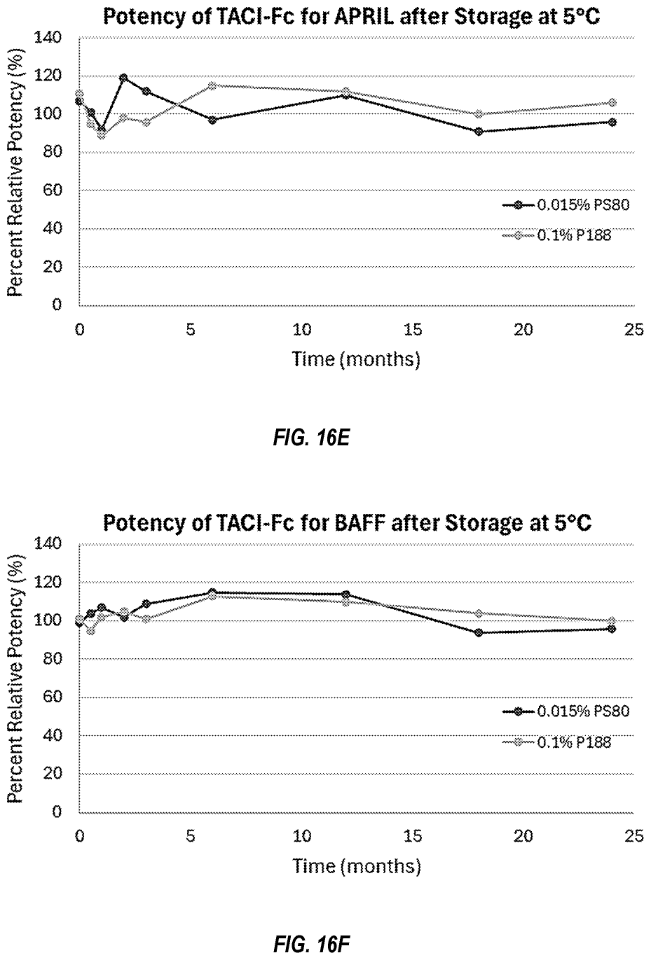

As shown in FIGS. 16A-F, TACI-Fc in formulation buffers containing either 0.015% PS80 and 0.1% P188 w/v retained activity for APRIL and BAFF for up to 104 weeks, which was the endpoint of the study, when stored at −70° C., −20° C., and 5° C. TACI-Fc in formulation buffers containing 0.015% PS80 or 0.1% P188 w/v also retained similar activity for APRIL and BAFF after storage at 25° C. and 40° C. for 6 months and 3 months, respectively.

### Summary

The results from Example 5, above, show that 100 mg/mL TACI-Fc formulated in a buffer containing 10 mM sodium acetate, 3% proline w/v with 0.015% polysorbate 80 (PS80) w/v or 0.1% Poloxamer 188 w/v at pH 5.2 show similar TACI-Fc stability when assessed by SEC, rCE-SDS, and icIEF; by light obscuration for subvisible particles; and by visual inspection for visible particles, color and clarity. The results also show that TACI-Fc formulated in this buffer in the presence of each of 0.015% polysorbate 80 (PS80) w/v or 0.1% Poloxamer 188 w/v retained activity for APRIL and BAFF. These results indicate that TACI-Fc can be formulated in a formulation buffer containing 10 mM sodium acetate, 3% proline w/v at pH 5.2 with either 015% polysorbate 80 (PS80) w/v or 0.1% Poloxamer 188 w/v and maintain stability, and activity for APRIL and BAFF.

### Example 6

### Further Assessing the Stability of a TACI-Fc Fusion Protein in Three Identified Formulation Buffers During Simulated Transport Conditions

Stability studies were carried out to assess the impact of the formulation buffer composition on the stability of a TACI-Fc fusion protein during simulated transport conditions by monitoring the properties of the TACI-Fc fusion protein homodimer and visual properties of the formulation. In these studies, three formulations that were shown in the preceding Examples to show TACI-Fc homodimer stability (formulations 21 and 39) were further assessed at pH 4.6 with different amounts of Poloxamer 188 surfactant. Formulation buffers containing: 10 mM sodium acetate, 3.1% arginine HCl (w/v) at pH 4.6; or 10 mM sodium acetate, 2% proline (w/v), 50 mM NaCl at pH 4.6, were prepared. Each of the formulations contained either 0, 0.01, 0.05, 0.1, or 0.3% w/v Poloxamer 188 and either 160 mg/mL or 175 mg/mL TACI-Fc.

A summary of the tested formulations is set forth in Table E18.

TABLE E18
Formulations Assessed for Stability
after Simulated Transport Conditions

| | | % | |
|---|---|---|---|
| | | Poloxamer | Amount |
| # | Formulation Components | 188 w/v | TACI-Fc |
| 48 | 10 mM sodium acetate, 3.1% arginine | 0 | 175 mg/mL |
| 49 | HCl (w/v) at pH 4.6 | 0.01 | |
| 50 | | 0.05 | |
| 51 | | 0.1 | |
| 52 | | 0.3 | |
| 53 | 10 mM sodium acetate, 2% proline (w/v), | 0 | 175 mg/mL |
| 54 | 50 mM NaCl at pH 4.6 | 0.01 | |
| 55 | | 0.05 | |
| 56 | | 0.1 | |
| 57 | | 0.3 | |
| 58 | 10 mM sodium acetate, 2% proline (w/v), | 0 | 160 mg/mL |
| 59 | 50 mM NaCl at pH 4.6 | 0.01 | |
| 60 | | 0.05 | |
| 61 | | 0.1 | |
| 62 | | 0.3 | |

Formulations were prepared essentially as detailed in Example 2.A. Formulations 48-62 were filled to a volume of 1.4 mL in a glass 2R vial (from Schott, product number 1557174) with stopper (from West, product number 19700004).

### A. Simulated Transport Conditions

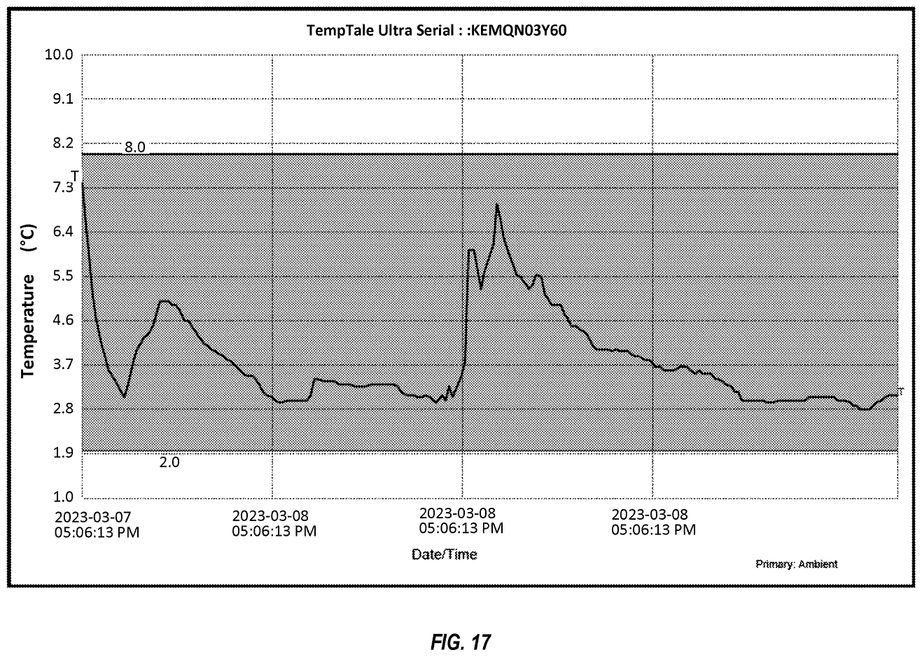

Samples containing the TACI-Fc in formulation buffers 48-62 were shipped using a NanoCool® shipping system which maintained temperature of the samples at about 2-15° C., and then kept at about 2-8° C. for the remainder of the experiment. Temperature (° C.) was monitored using the TempTale Ultra® temperature monitoring system over 48 hours during shipping in the NanoCool® shipping system. The results show that temperature varied between about 2.8° C. and about 7° C. for the transport, which is within the desired range of 2-8° C. Recorded temperatures (° C.) are set forth in FIG. 17.

Samples containing the TACI-Fc in formulation buffers 48-62 were shipped to a testing facility, subjected to a series of drops and vibrations in accordance with the ASTM D4169-22 Standard Practice for Performance Testing of Shipping Containers and Systems, then shipped back to the analytical testing facility for characterization.

### B. Characterization of TACI-Fc Stability in Different Formulations after Simulated Transport

The properties and stability of the TACI-Fc homodimer in the formulation buffers 48-62 was assessed after storage using the assays set forth in Table E19, below:

TABLE E19
Assays for Assessing TACI-Fc Stability

| Designator | Assay |
|---|---|
| A | SEC |
| B | Protein Concentration |
| C | Potency |
| D | Clarity |
| E | Subvisible Particles and Visible Particles |

### 1. Size Exclusion Chromatography (SEC)

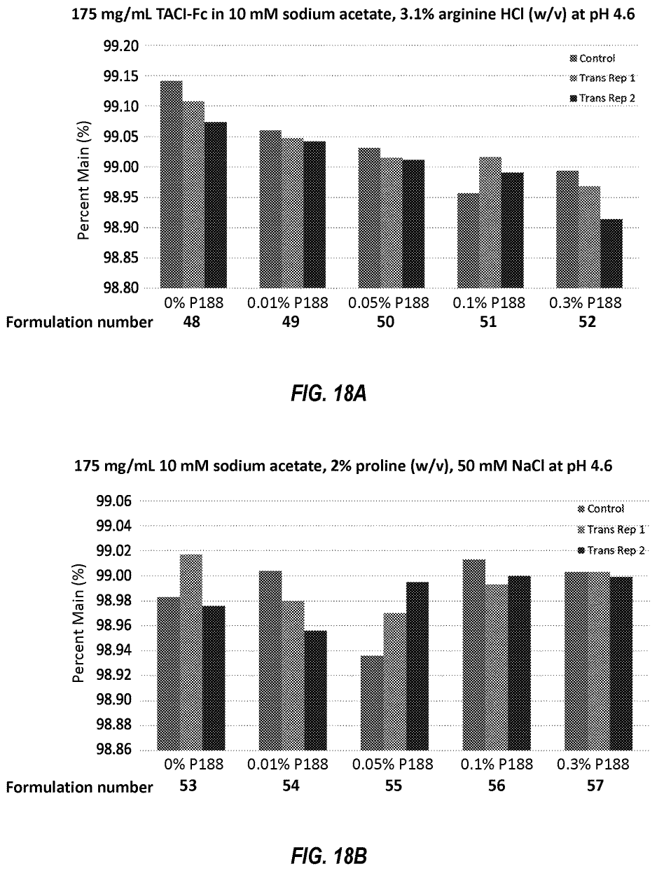

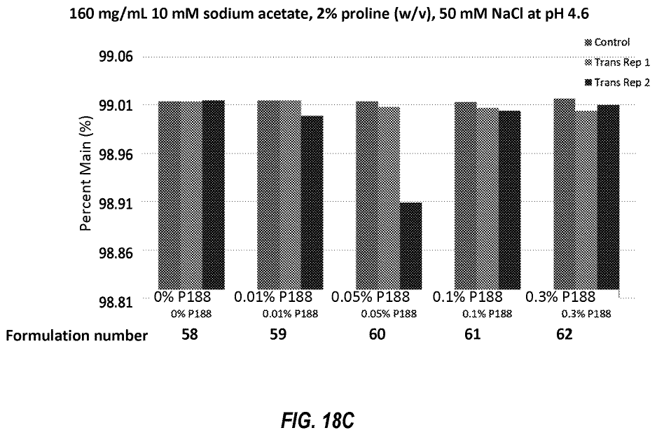

SEC was used to assess stability and the rate of TACI-Fc aggregation and fragmentation in formulations 48-62 as detailed in Example 2. Using SEC, the percent main of TACI-Fc in each of the formulations set forth in Table E18 was assessed. In this simulated transport study, there were negligible differences in the percent TACI-Fc main species in the transport samples compared to the control samples that were not subjected to the simulated transport conditions, as set forth in FIGS. 18A-C. In FIGS. 18A-C, the first bar in each set of three bars is the control, which was not subjected to the simulated transport, and the second and third bars are replicates 1 and 2 (Trans Rep 1 and Trans Rep 2, respectively).

### 2. TACI-Fc Protein Concentration

To assess whether physical agitation impacts the protein concentration in the formulation after transport, the concentration of the TACI-Fc in the formulations after simulated transport was also assessed. Protein concentration of simulated transport condition test samples was assessed by variable pathlength UV-Vis spectroscopy based upon the Beer-Lambert law with an extinction coefficient of 1.25 mL/(mg*cm) at 280 nm. Absorbance readings were taken at variable pathlengths with the protein concentration calculated from the slope of an absorbance vs. pathlength plot. The results show only negligible changes in protein concentration between all conditions; all conditions maintained a protein concentration within about 300 of the target protein concentration, as shown in Table E20. The results indicate that simulated transport conditions, including refrigerated temperature and physical shocks associated with transport, did not alter TACI-Fc protein concentration in any of the tested formulation buffers compared to controls that were not subjected to the simulated transport.

TABLE E20
TACI-Fc Protein Concentration after Simulated Transport

| | | Protein |
|---|---|---|
| Formulation | Sample | Concentration (mg/mL) |
| 48 | Control | 177 |
| | Transport Round 1 | 178 |
| | Transport Round 2 | 178 |
| 49 | Control | 179 |
| | Transport Round 1 | 178 |
| | Transport Round 2 | 180 |
| 50 | Control | 174 |
| | Transport Round 1 | 176 |
| | Transport Round 2 | 177 |
| 51 | Control | 177 |
| | Transport Round 1 | 178 |
| | Transport Round 2 | 175 |
| 52 | Control | 176 |
| | Transport Round 1 | 173 |
| | Transport Round 2 | 176 |
| 53 | Control | 174 |
| | Transport Round 1 | 172 |
| | Transport Round 2 | 173 |
| 54 | Control | 173 |
| | Transport Round 1 | 174 |
| | Transport Round 2 | 170 |
| 55 | Control | 172 |
| | Transport Round 1 | 171 |
| | Transport Round 2 | 173 |
| 56 | Control | 173 |
| | Transport Round 1 | 172 |
| | Transport Round 2 | 171 |
| 57 | Control | 170 |
| | Transport Round 1 | 169 |
| | Transport Round 2 | 170 |
| 58 | Control | 158 |
| | Transport Round 1 | 159 |
| | Transport Round 2 | 159 |
| 59 | Control | 160 |
| | Transport Round 1 | 157 |
| | Transport Round 2 | 160 |
| 60 | Control | 158 |
| | Transport Round 1 | 157 |
| | Transport Round 2 | 157 |
| 61 | Control | 159 |
| | Transport Round 1 | 159 |
| | Transport Round 2 | 158 |
| 62 | Control | 158 |
| | Transport Round 1 | 156 |
| | Transport Round 2 | 156 |

### 3. Subvisible Particle Assessment

A HIAC Liquid Particle Counter (Beckman Coulter) particle analyzer was used to measure subvisible particles for all samples after simulated transport. Assessments were conducted in accord with the manufacturer's instructions to count the subvisible particles in the samples as detailed in Example 5. USP guidelines specify no more than 6,000 ≥10 μm particles per container; and no more than 600 ≥25 μm particles/container. The cumulative number of particles 2 μm or ≥10 μm or ≥25 μm per container, were counted.

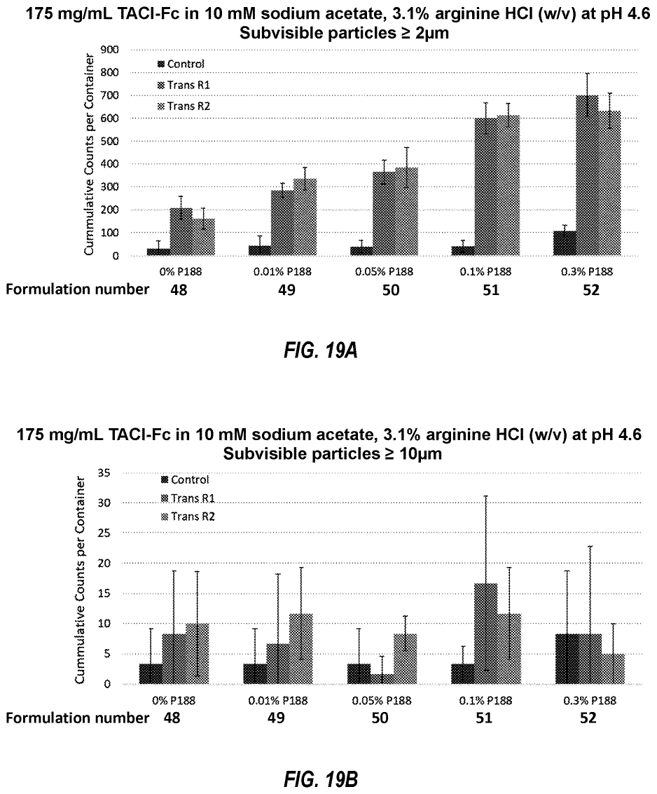

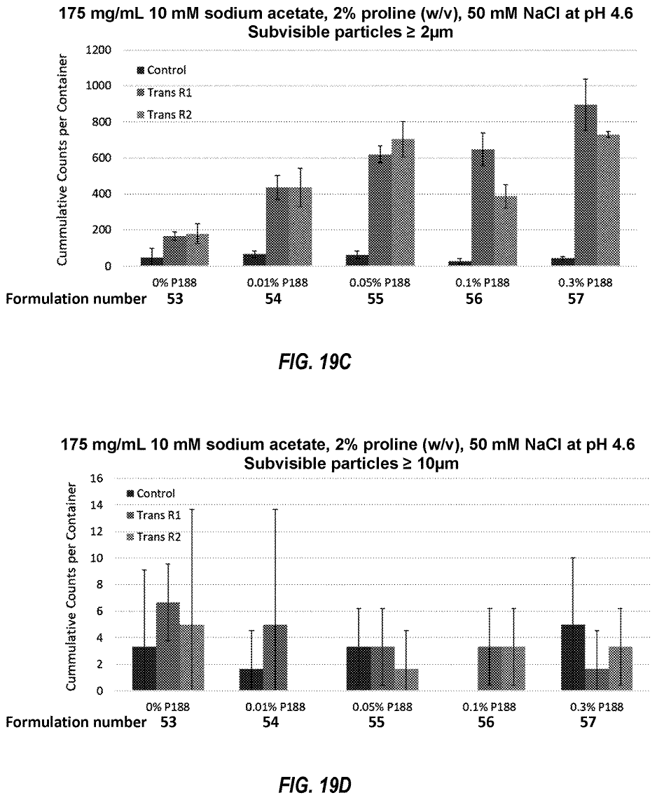

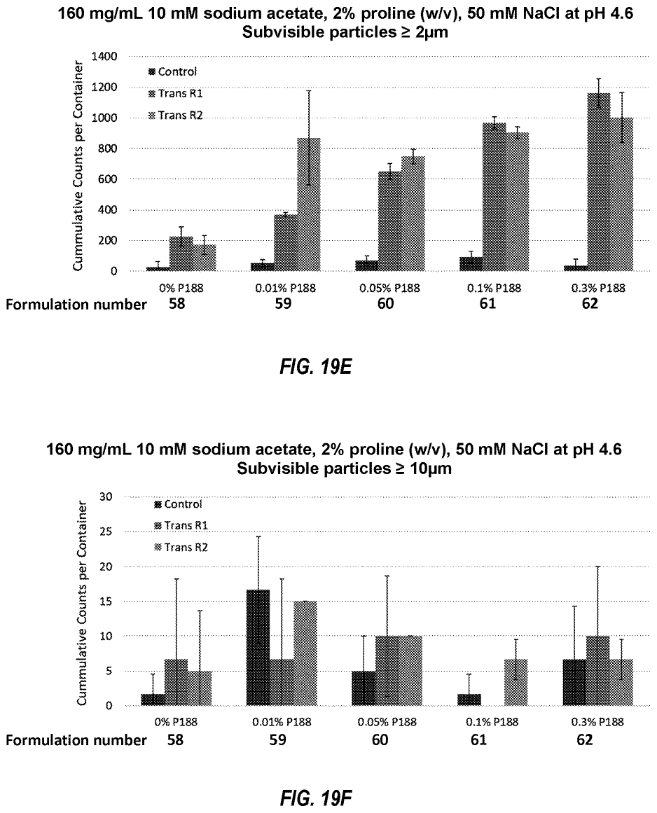

For all formulations the number of subvisible particles after simulated transport increased compared to controls that were not subject to simulated transport. FIGS. 19A and 19B show that the number of ≥2 μm and ≥10 μm particles increased after transport for formulation buffers containing 10 mM sodium acetate, 3.1% arginine HCl (w/v) and all amounts of surfactant at pH 4.6, although the increase was not significant for the ≥10 μm particles and was variable. FIGS. 19C and 19D show that the number of ≥2 μm particles increased after transport for formulation buffers containing 175 mg/mL TACI-Fc in 10 mM sodium acetate, 2% proline (w/v), 50 mM NaCl and all amounts of surfactant at pH 4.6, and the number of ≥10 μm particles was not impacted by transport. FIGS. 19E and 19F show that the number of ≥2 μm particles increased after transport for formulation buffers containing 160 mg/mL TACI-Fc in 10 mM sodium acetate, 2% proline (w/v), 50 mM NaCl and all amounts of surfactant at pH 4.6, and the number of ≥10 μm particles was not impacted by transport. There was a slight dependence on poloxamer concentration; the number of ≥2 μm particles increased with poloxamer concentration in all three formulations. The highest number of ≥2 μm particles was in the formulations containing 0.3% Poloxamer 188 (formulations 52, 57 and 62) and subject to simulated transport. The results show that the amount of poloxamer can impact the number of subvisible particles ≥2 μm that form after simulated transport, with the greatest increase seen in formulations containing the highest amount of surfactant (0.3%). The results also show that the subvisible particle counts were within USP guidelines for all conditions.

### 4. Visual Inspection

Stability of the TACI-Fc formulations also was assessed by visual inspection after simulated transport. The formulations were assessed for color, clarity, and the presence of visible particles as detailed in Example 5, above. All of formulations 48-62 have a color of <BY4 and all formulations were considered “clear.” The results from assessing visible particles show that all formulations without Poloxamer 188 had many entrapped microbubbles and/or particles, and all formulations containing Poloxamer 188 were essentially free of visible particles. In formulations containing Poloxamer 188, 0-2 particles or fibers were rarely observed. Formulation in the absence of Poloxamer 188 surfactant resulted in many entrapped microbubbles and/or particles, whereas formulations containing surfactant rarely had any visible fibers or particles.

### 5. TACI-Fc Anti-APRIL and Anti-BAFF Activity

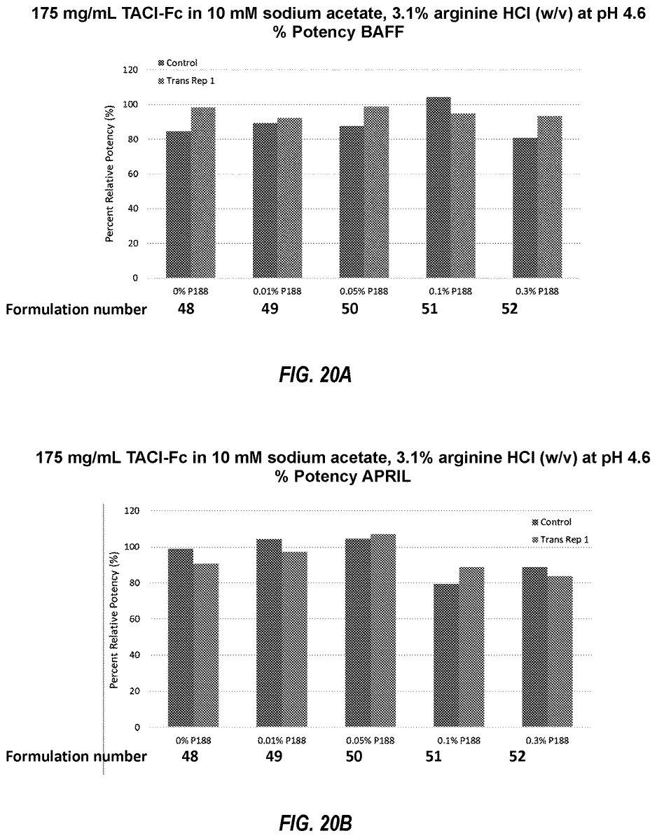

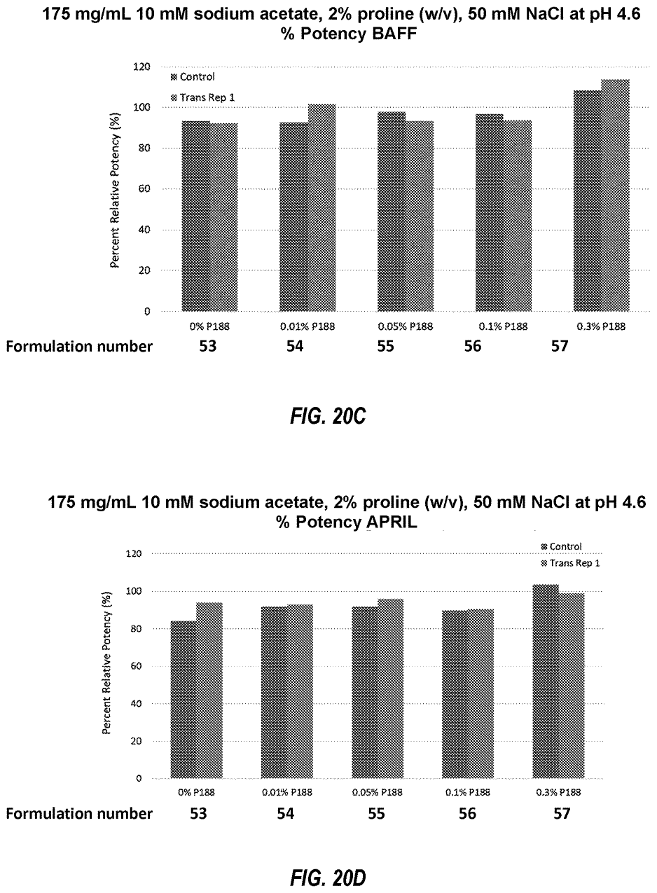

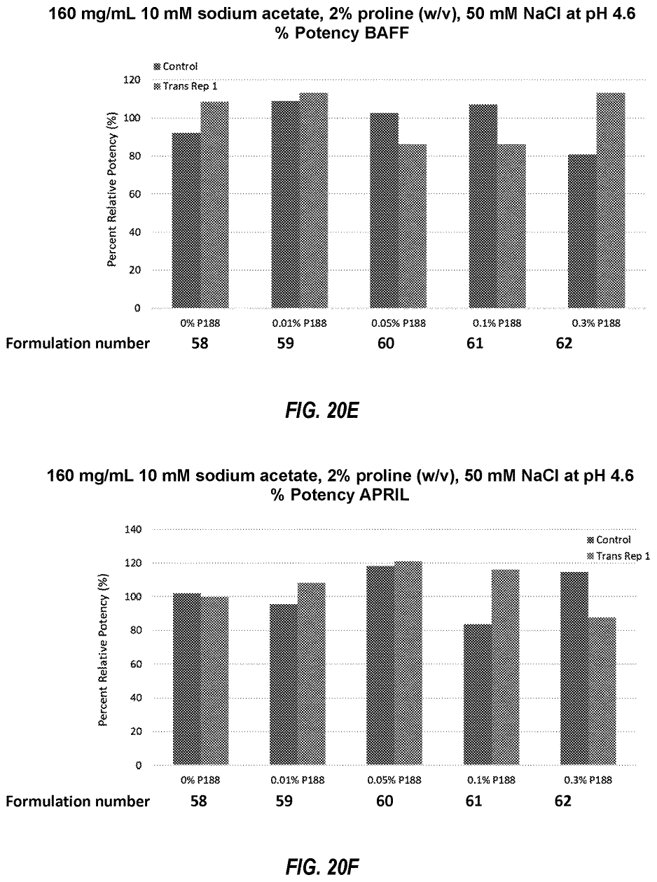

Activity of the TACI-Fc for APRIL and BAFF also was assessed as detailed in Example 5, above. As shown in FIGS. 20A-F, the TACI-Fc in all formulation buffers retained similar activity for APRIL and BAFF after simulated transport compared to non-transport controls. No significant differences between conditions were observed.

### Summary

The results in this example show that TACI-Fc formulated in a buffer containing 10 mM sodium acetate, 3.1% arginine HCl (w/v) at pH 4.6 and 175 mg/mL TACI Fc or 10 mM sodium acetate, 3% proline w/v and 10 mM NaCl and 160 mg/mL or 175 mg/mL TACI Fc, with either 0, 0.01, 0.05, 0.1, or 0.3% w/v Poloxamer 188 at pH 5.2 show similar simulated transport stability when assessed by SEC, by protein concentration, by analysis for activity of the TACI-Fc for APRIL and BAFF, by analysis for subvisible and visible particle formation, and by visual analysis for color and clarity. There was a slight increase in the number of ≥2 μm subvisible particles that form after simulated transport in formulations containing 0.3% w/v P188 compared to formulations containing less than 0.3% w/v P188. Also, formulation preparation in the absence of surfactant can impact visual inspection, as all formulations without poloxamer 188 had many entrapped microbubbles and/or particles when assessed visually. These results show that increasing P188 concentration up to 0.3% w/v can impact stability after transport, and lack of surfactant can impact properties of the TACI-Fc. The TACI-Fc formulated in Formulations 48-62 in the presence of Poloxamer 188 retained similar activity for APRIL and BAFF after simulated transport. These results indicate that TACI-Fc fusion protein can be formulated in these buffers and remain stable and active after simulated transport.

### Example 7

### Further Assessing the Long-Term Stability of a TACI-Fc Fusion Protein in Three Identified Formulation Buffers

Additional studies were carried out to monitor and assess the long-term stability of a TACI-Fc fusion protein in different formulation buffers at different temperatures. In these studies, three formulations identified in the preceding Examples that showed a stable TACI-Fc homodimer were prepared and assessed. The stability of the TACI-Fc fusion protein in the different buffers after prolonged storage at either −70° C., −20° C., 2-8° C., 25° C., or 40° C. for up to 52 weeks, was assessed using the assays described in Examples 2 and 5, above.

A summary of the tested formulations is set forth in Table E21.

TABLE E21
Formulations Assessed for Stability

| | | % | |
|---|---|---|---|
| | | Poloxamer | Amount |
| # | Formulation Components | 188 w/v | TACI-Fc |
| 51 | 10 mM sodium acetate, 3.1% arginine HCl | 0.1 | 175 mg/mL |
| | (w/v) at pH 4.6 | | |
| 56 | 10 mM sodium acetate, 2% proline (w/v), | 0.1 | 175 mg/mL |
| | 50 mM NaCl at pH 4.6 | | |
| 61 | 10 mM sodium acetate, 2% proline (w/v), | 0.1 | 160 mg/mL |
| | 50 mM NaCl at pH 4.6 | | |

Formulations were prepared essentially as detailed in Example 2.A. Formulations were filled to a volume of 1.4 mL in a glass ISO2R adptiQ vial (from Scott, product number 1557174) with 12 mm chlorobutyl stopper (from West, product number 19700004).

### A. Characterization of TACI-Fc Stability in Different Formulations

The properties and stability of the TACI-Fc in the three formulation buffers after prolonged storage at different temperatures was assessed using the assays summarized in Table E22.

TABLE E22
Assays for Assessing TACI-Fc Stability

| Designator | Assay |
|---|---|
| A | SEC, rCE-SDS, icIEF, and/or potency against APRIL and |
| | BAFF |
| B | Color, clarity, visible particles, subvisible particles |
| | (HIAC) |
| C | pH, retains for MAM |
| D | Protein concentration |
| E | Osmolality |

A summary of the stability assays, storage temperature, and storage times assessed in this study is set forth in Table E23.

TABLE E23
Conditions Tested and Assays for Each Condition and Timepoint

| | Temperature | | | | |
|---|---|---|---|---|---|
| Time | −70° C. | −20° C. | 2-8° C. | 25° C. | 40° C. |
| 2 weeks | AB | AB | AB | AB | AB |
| 4 weeks | AB | AB | AB | AB | AB |
| 8 weeks | AB | AB | AB | AB | AB |
| 3 months | AB | AB | AB | AB | ABCD |
| 6 months | ABD | ABD | ABD | ABCD | |
| 12 months | ABD | ABD | ABD | | |

As set forth in Table E22 and E23, above, TACI-Fc stability was assessed by SEC, rCE-SDS, and icIEF after storage at −70° C., −20° C., 2-8° C., 25° C., or 40° C. Some SEC, rCE, and icIEF samples were frozen and thawed before assaying. Data collected out to the 52 week time point contained 7 data points, samples incubated at 25° C. were incubated a maximum of 26 weeks (6 data points), and samples incubated at 40° C. were incubated a maximum of 12 weeks (5 data points). Rate was calculated assuming a linear relationship, and error bars are the calculated standard error in the rate calculation.

### 1. Size Exclusion Chromatography (SEC)

SEC was used to assess stability and the rate of aggregation and the forms of the TACI-Fc protein in each of the three formulations set forth in Table E21 as detailed in Example 2. Using SEC, the percentage of the protein that was the main species or a HMW TACI-Fc species was assessed after long term storage. The formulations were incubated at −70° C., −20° C., 5° C., 25° C., and 40° C. as indicated in Table E23, above, and the percentage of main, low molecular weight (LMW) or high molecular weight (HMW) species was assessed. The rate of change in the percent of the different species in the formulation was calculated as detailed in Example 2.

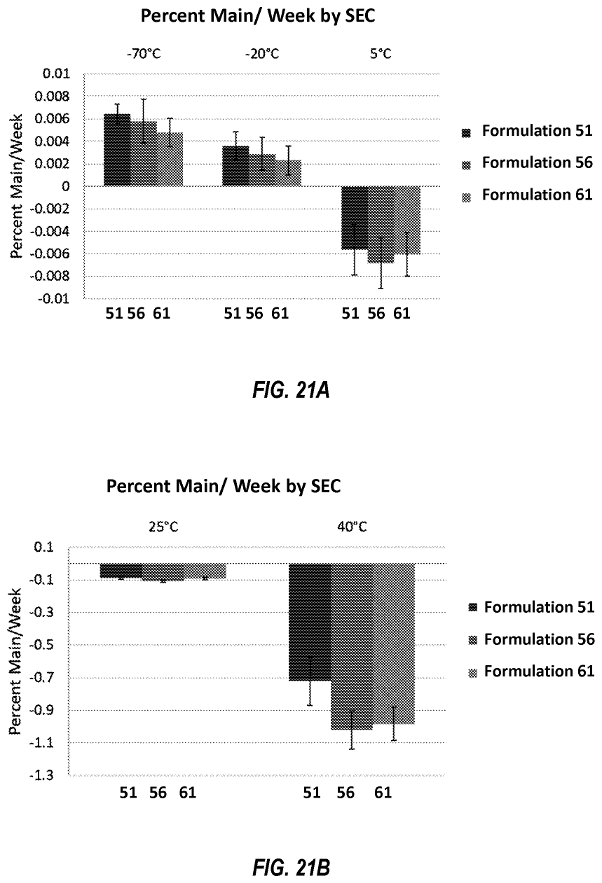

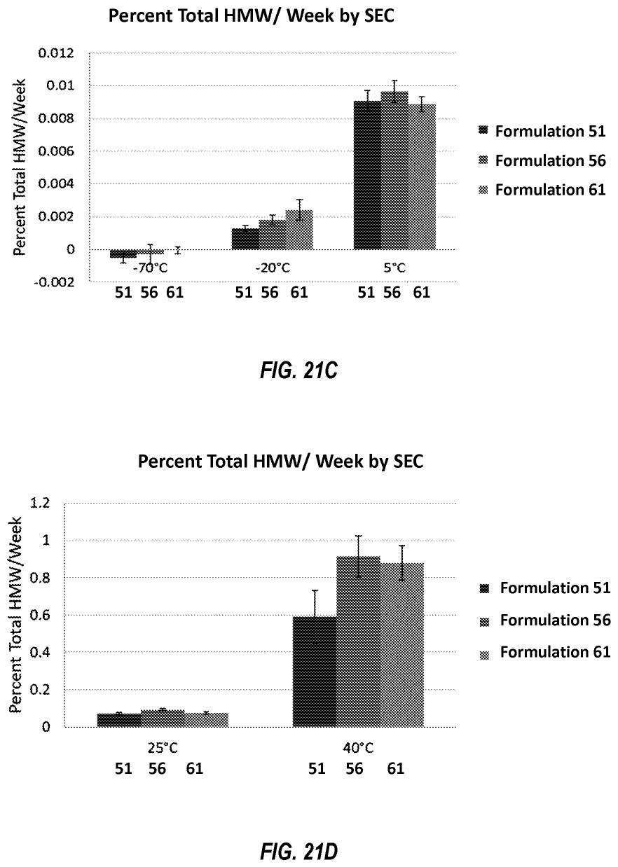

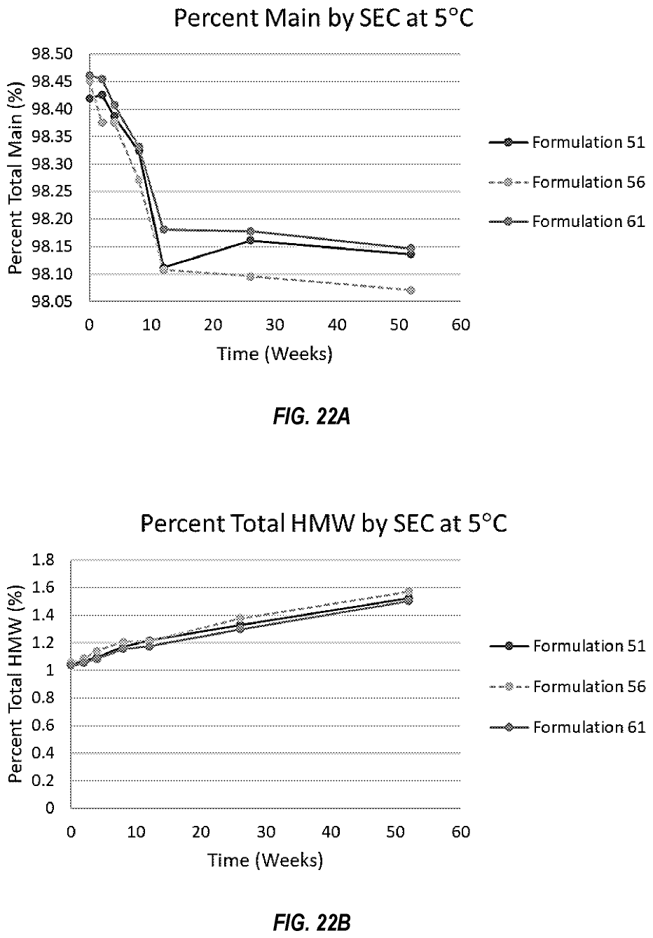

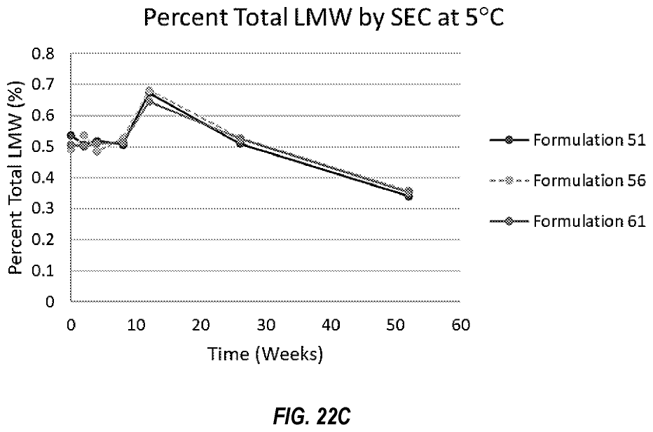

As set forth in FIGS. 21A-D and FIGS. 22 A-C, there were negligible differences between the formulations in the rate of change of the TACI-Fc percent main and HMW TACI-Fc species over time at −70° C., −20° C., and 5° C. The SEC results show that for all conditions the percent of the TACI-Fc homodimer (% main) decreased and the percent of HMW species increased upon storage at increasing temperatures. There was slightly smaller decrease in percent main and extent of formation of HMW species of the TACI-Fc after incubation at 40° C. in the buffer containing 10 mM sodium acetate, 3.1% arginine HCl (w/v), and 0.1% Poloxamer 188 w/v at pH 4.6 (Formulation 51) compared to the same incubation in a proline buffer (Formulation 56 or 61). The increase in percent of HMW species per week after storage at 5° C. was approximately 0.009% HMW per week for all conditions, which equates to an increase of approximately 0.5% after 1 year.

### 2. Reduced Capillary Electrophoresis Sodium Dodecyl Sulfate (rCE-SDS)

Using rCE-SDS assays as detailed in Example 5, the percent decrease in the TACI-Fc homodimer (main species) was assessed after preparation in the 3 formulation buffers in Table E21 after storage for 52 weeks at −70° C., −20° C., and 5° C., 26 weeks at 25° C., and 12 weeks at 40° C. Data was collected at 7 times points over the 52-week period during incubation at −70° C.,−20° C., and 5° C., and 6 timepoints during incubation at 25° C. and 5 timepoints during incubation at 40° C. and the rate of the loss of the TACI-Fc homodimer (main species) was calculated per week over the defined period.

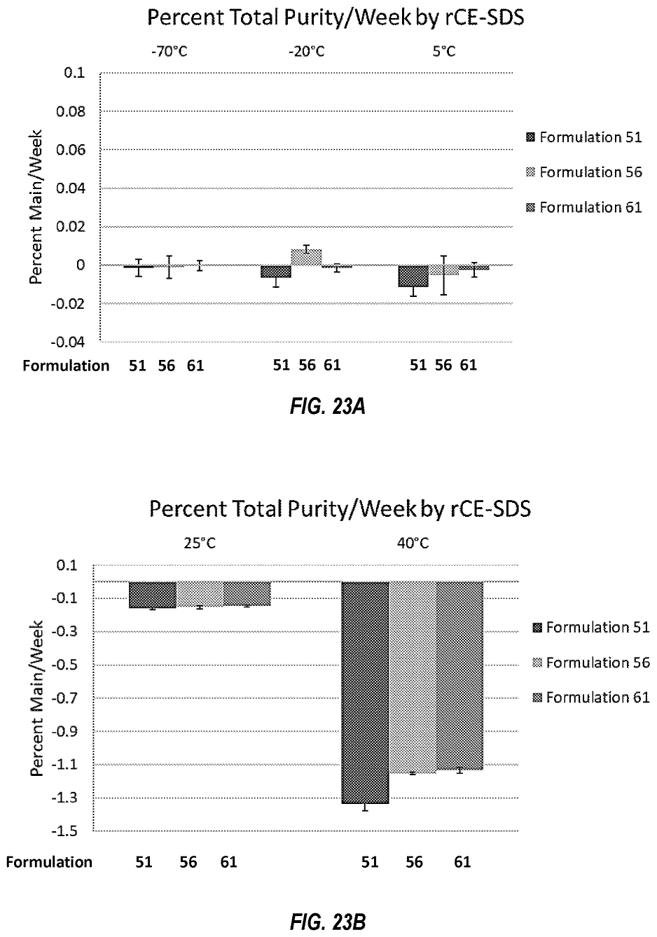

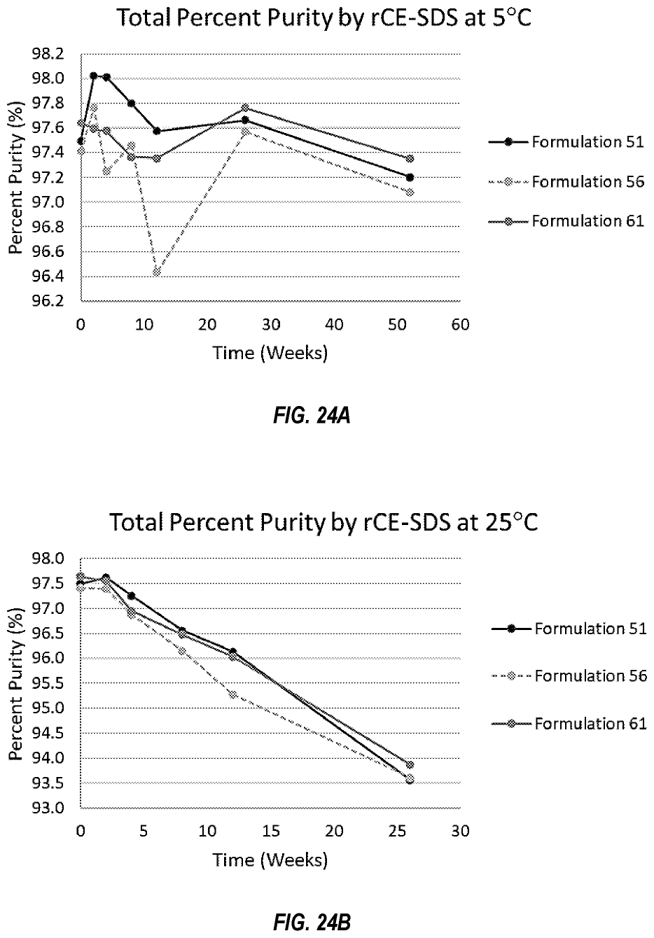

As assessed by rCE-SDS, the results were similar to the SEC results and show that TACI-Fc main species rate of degradation is similar in all formulations after long term storage at −70° C., −20° C., 5° C., and 25° C. as shown in FIGS. 23A-B. However, after incubation for 3 months at 40° C., there was less TACI-Fc main species in the buffer containing 10 mM sodium acetate, 3.1% arginine HCl (w/v), and 0.1% Poloxamer 188 w/v at pH 4.6 (Formulation 51) compared to the same incubation in a proline buffer (Formulation 56 or 61). The loss of TACI-Fc main species per week after storage at 5° C. was approximately 0.011% per week for all conditions, which equates to a loss of approximately 0.6% after 1 year. As shown in FIGS. 24A and 24B, the results from the rCE-SDS experiments show the percent of TACI-Fc main species in the formulations decreased over time after incubation for 52 weeks at 5° C. and 26 weeks at 25° C. After incubation for 52 weeks at 5° C. and 26 weeks at 25° C., there was a decrease of about 0.6% and 3-3.5% of TACI-Fc main species, respectively.

### 3. Imaged Capillary Isoelectric Focusing (icIEF)

Using icIEF assays as detailed in Example 5, the percent decrease in the main TACI-Fc species was assessed after preparation in the 3 formulation buffers in Table E21 after storage for 52 weeks at −70° C., −20° C., and 5° C., 26 weeks at 25° C., and 12 weeks at 40° C. Data was collected at 7 times points over the 52-week period during incubation at −70° C., −20° C., and 5° C., and 6 timepoints during incubation at 25° C. and 5 timepoints during incubation at 40° C. and the rate of the loss of the main TACI-Fc species was calculated per week over the defined period. The rate was calculated assuming a linear relationship and error bars represent the standard error in the rate calculation.

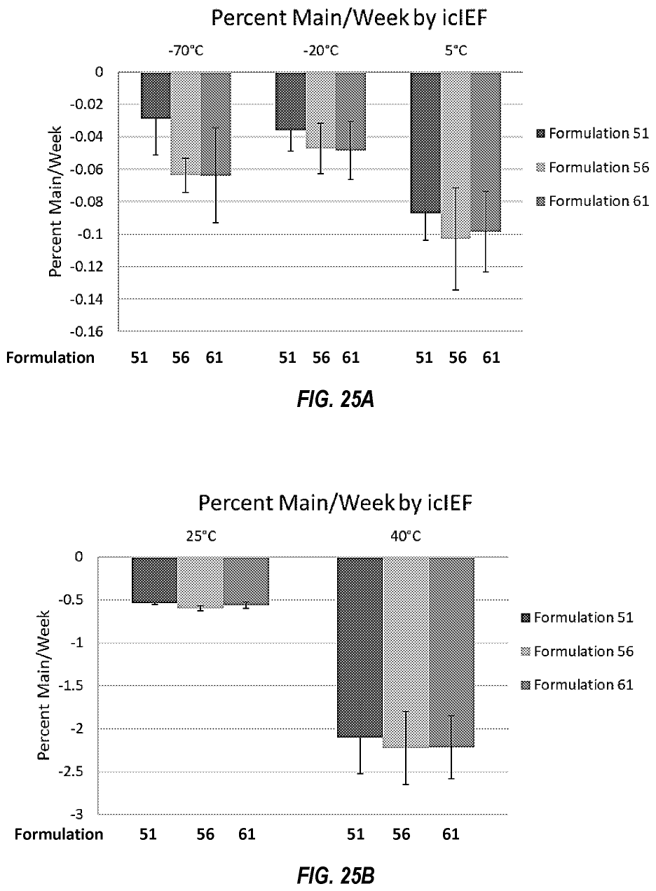

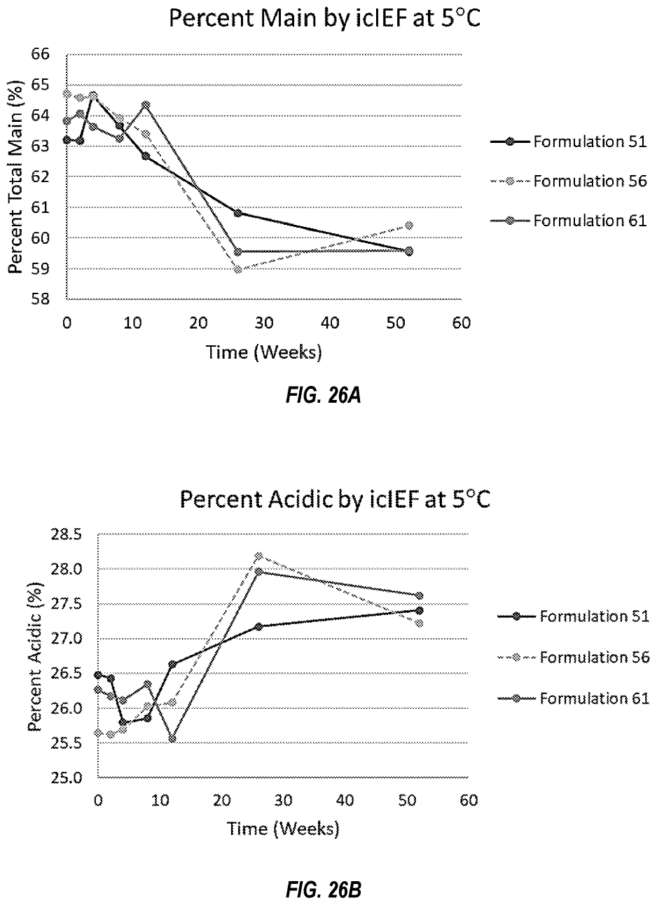

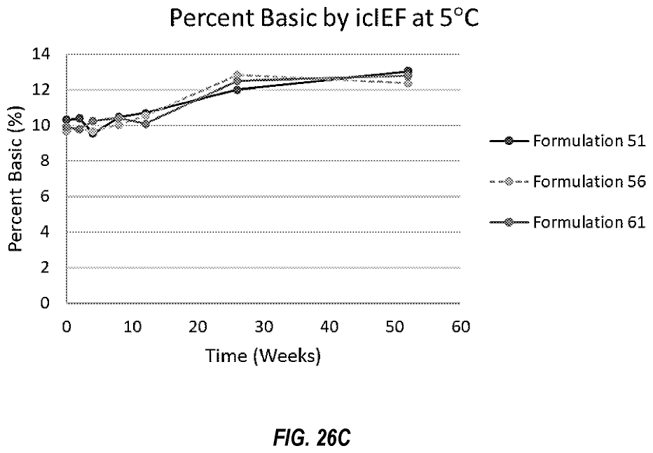

As shown in FIGS. 25A, 25B and 26A, the results after assessment of the change in percent main by icIEF show that there was not a significant difference between formulation buffers in the main rate of the TACI-Fc after storage for 52 weeks at −70° C., −20° C., and 5° C. However, by icIEF after incubation for 12 weeks at 40° C., the rate of degradation of the TACI-Fc main in the buffer containing 10 mM sodium acetate, 3.1% arginine HCl (w/v), and 0.1% Poloxamer 188 w/v at pH 4.6 (Formulation 51) trended lower compared to the same incubation in a proline buffer (Formulation 56 or 61). The charge profile of the TACI-Fc was assessed by imaged capillary isoelectric focusing (icIEF) as detailed in Example 5. As shown in FIGS. 26B and 26C, the results show that there was not a significant difference between formulation buffers in the percent basic (% basic) or percent acidic (% acidic) variants of the TACI-Fc after storage for 52 weeks at 5° C.

### 4. Subvisible Particle Assessment

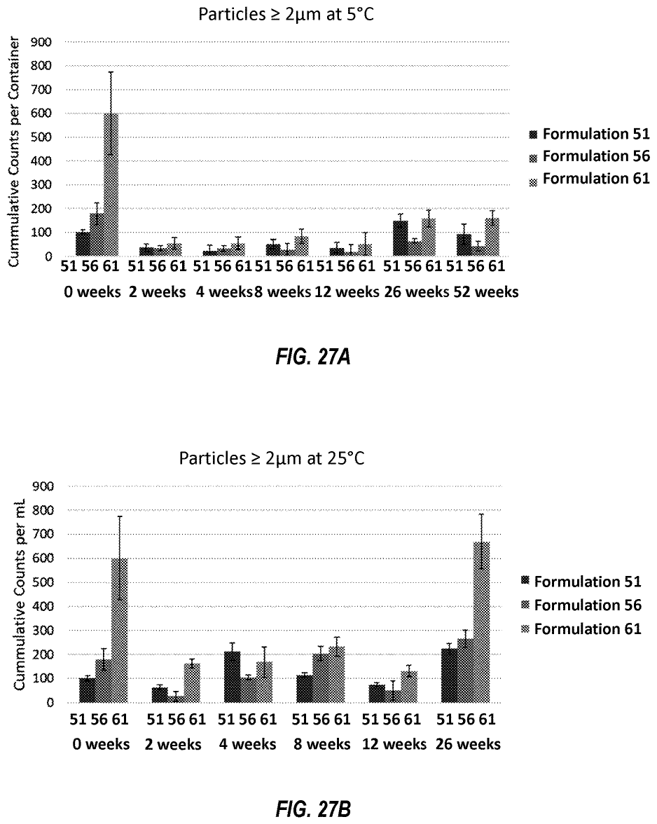

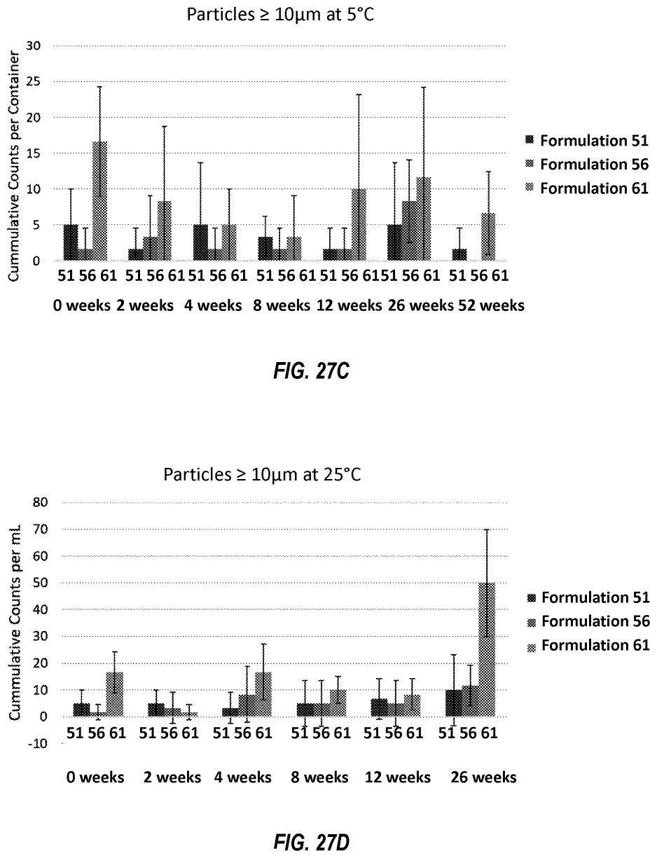

Stability also was assessed, as detailed in Example 5, using a HIAC Liquid Particle Counter (Beckman Coulter) particle analyzer to assess the number of subvisible particles of the TACI-Fc formulated in the three formulation buffers after storage for up to 52 weeks at 5° C. and up to 26 weeks at 25° C. The cumulative number of particles ≥2 μm or ≥10 μm per container, was counted. FIGS. 27A-D show the counts for ≥2 μm or ≥10 μm particles by HIAC analysis over 52 week incubation at 5° C. and 26 week incubation at 25° C. Negligible differences between formulations were observed in the number of subvisible particles in TACI-Fc formulations after incubation for up to 52 weeks at 5° C. and up to 26 weeks at 25° C.

### 5. Visual Inspection

Stability of the TACI-Fc also was assessed by visual inspection after prolonged storage of the three formulations at different temperatures. The three formulations were assessed by eye for color, clarity, and the presence of visible particles after storage at −70° C., −20° C., 2-8° C., 25° C., and 40° C. for up to 52 weeks as described in Example 5.

The color of the prepared TACI-Fc formulations was visually inspected. All formulations showed a color of ≤BY4 after storage at −70° C., −20° C., and 5° C. for up to 52 weeks and ≤BY4 at 25° C. for up to 6 months. Formulations 51 and 56 underwent a shift to <BY3 after storage at 40° C. for 8 weeks, and formulation 61 underwent a shift to <BY3 after storage at 40° C. for 12 weeks.

The relative clarity of the prepared TACI-Fc formulations was visually inspected. All formulations showed a clarity of “clear” after storage at −70° C., −20° C., and 5° C. for up to 52 weeks. Formulation 51 showed a clarity of <RSII after 12 weeks at 25° C. and after 2 weeks at 40° C., and <RSIII after 4 weeks at 40° C., and <RSIV after 12 weeks at 40° C. Formulations 56 and 61 showed a clarity of <RSII after 8 weeks at 40° C.

These results show that the tested formulations demonstrated a temperature and time dependent clarity and color shift.

The presence of visible particles in the prepared TACI-Fc formulations was assessed by visible inspection. All formulations were essentially free of visible particles for all time points and temperatures studies. A single fiber or particle was rarely observed.

### 6. TACI-Fc Anti-APRIL and Anti-BAFF Activity

Activity of the TACI-Fc for APRIL and BAFF also was assessed as detailed in Example 6. The bioactivity of the TACI-Fc protein stored in each of formulation buffers 51, 56, and 61 at −70° C., −20° C., 4-8° C., 25° C., and 40° C. was assessed.

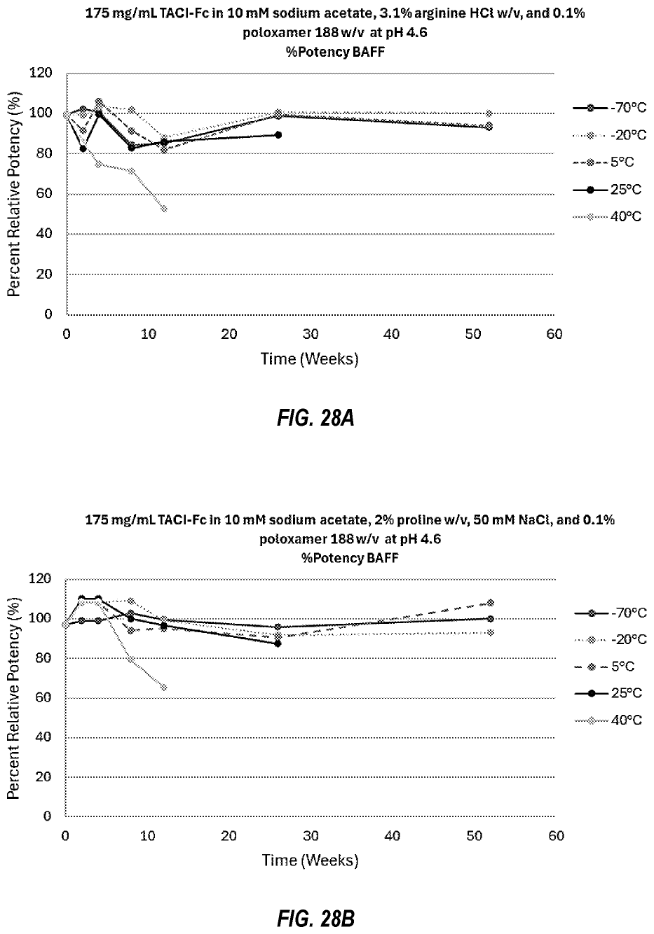

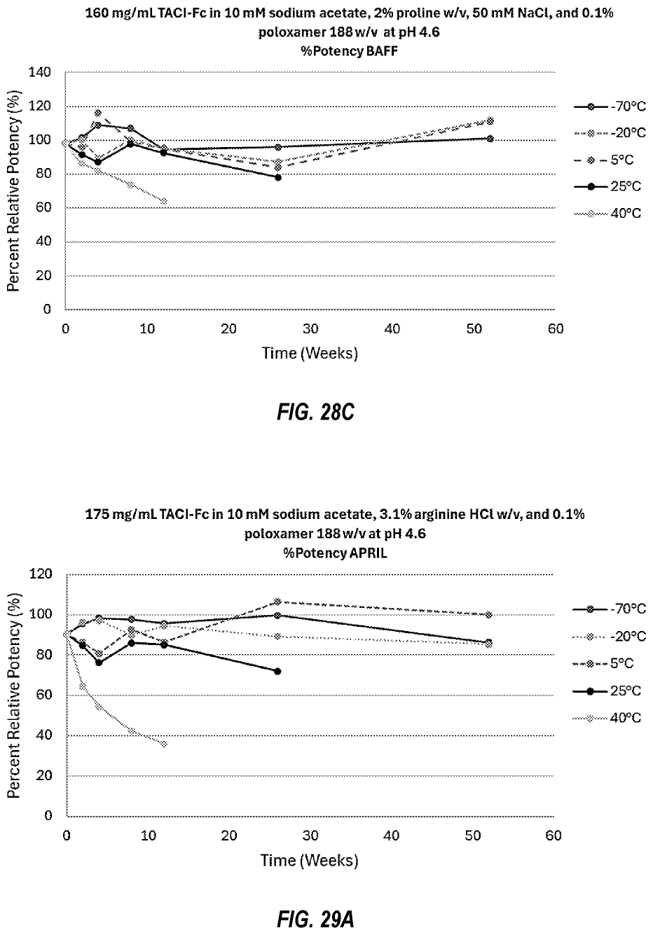

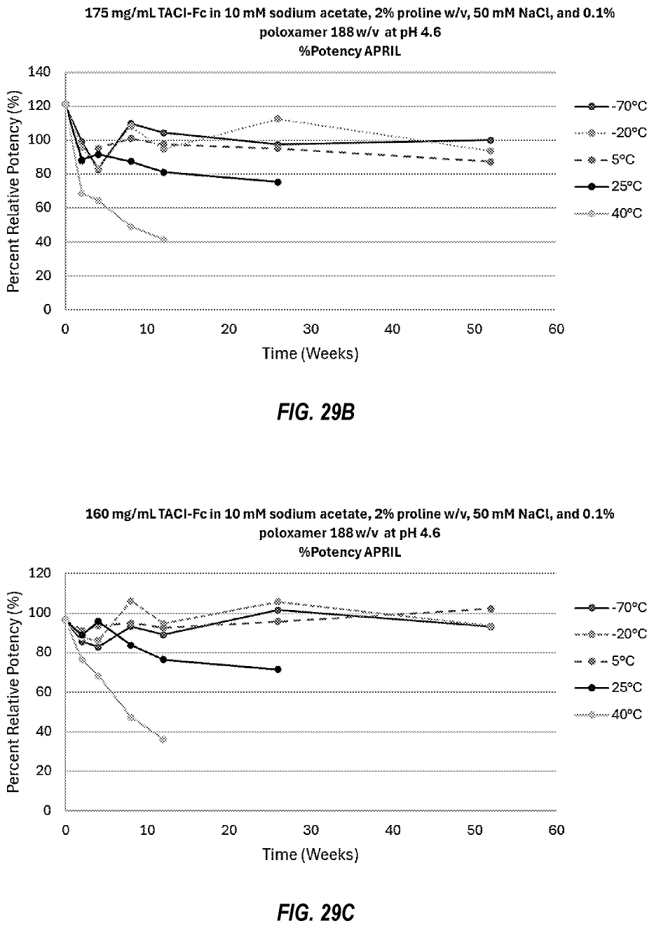

As shown in FIGS. 28A-28C and FIGS. 29A-29C, TACI-Fc formulated in formulation buffers 51, 56, and 61 retained activity for APRIL and BAFF for up to 52 weeks, when stored at −70° C., −20° C., and 5° C. TACI-Fc formulated in formulation buffers 51, 56, and 61 also retained similar activity for APRIL and BAFF after storage at 25° C. for 26 week. After prolonged storage at 40° C. for 12 weeks, the TACI-Fc formulated in all buffers showed a decreased activity for both APRIL and BAFF. These results show that TACI-Fc stored in each of formulation buffers 51, 56 and 61 can maintain activity for APRIL and BAFF after prolonged storage at lower temperatures, but in all buffers the activity for APRIL and BAFF decreases after prolonged storage at high temperature of 40° C.

### Summary

The results show that TACI-Fc formulated in formulation buffers 51, 56 and 61, which were prepared at pH 4.6 and at high concentrations (160 mg/mL and 175 mg/mL) of TACI-Fc, show similar stability when assessed by SEC, rCE-SDS, or icIEF; for subvisible particles by light obscuration; and by visual inspection for visible particles, color and clarity at 2-8° C. The results also show that TACI-Fc formulated in these buffers retained activity for APRIL and BAFF. These results indicate that TACI-Fc can be formulated at a high concentration and low pH in buffers containing the identified components and maintain stability and activity at 2-8° C.

### Example 8

### Further Assessing the Stability of a TACI-Fc Fusion Protein in an Identified Formulation Buffer

In the Examples above, the TACI-Fc showed high stability in Formulation 51, containing 10 mM sodium acetate, 3.1% arginine HCl (w/v) and 0.1% Poloxamer 188 w/v at pH 4.6. In this Example, the pH, API concentration, Arg-HCl and P188 concentrations of Formulation 51 were varied to assess their impact on long-term stability of a TACI-Fc fusion protein. Studies monitoring the effect of different amounts of the amino acid (arginine HCl), surfactant (poloxamer 188), and TACI-Fc protein in a range of pH were conducted. The studies were carried out using arginine HCl as an excipient at pH at or below 5.0 with varying amounts of surfactant, based on initial stability results in Examples 1-4, 6 and 7.

In these studies, formulations at one of four pH values (pH 4.3, 4.4, 4.6 and 5.0), one of three amounts of Arg-HCl (2.79%, 3.1%, and 3.41% w/v), and one of three poloxamer 188 amounts (0.04%, 0.16% and 0.1% w/v) were tested. Each formulation was also designed to contain a high and low concentration of TACI-Fc protein; protein concentration of the tested formulations was 158, 175 or 193 mg/mL. All of the formulations contained 10 mM sodium acetate as the initial buffering agent. A summary of the thirteen tested formulations is set forth in Table E24, below.

TABLE E24
Formulations

| | | Arg | | Target Protein | Target |
|---|---|---|---|---|---|
| Formula- | | HCl | Target | Concentration | Poloxamer |
| tion | Buffer | (% w/v) | pH | (mg/mL) | 188 (% w/v) |
| 62 | 10 mM | 3.1 | 4.3 | 158 | 0.04 |
| 63 | sodium | | | 193 | 0.04 |
| 64 | acetate | | | 158 | 0.16 |
| 65 | | | | 193 | 0.16 |
| 66 | | | 4.6 | 175 | 0.1 |
| 67 | | | | 175 | 0.1 |
| 68 | | | 5.0 | 158 | 0.04 |
| 69 | | | | 193 | 0.04 |
| 70 | | | | 158 | 0.16 |
| 71 | | | | 193 | 0.16 |
| 72 | | 2.79 | 4.6 | 175 | 0.1 |
| 73 | | 3.41 | | | |
| 74 | | 3.1 | 4.4 | 175 | 0.1 |

Formulations 62-74 as set forth in Table E24, containing the various excipients and at the listed pH values, were prepared as detailed in Example 2 where the TACI-Fc fusion protein was concentrated using cassette filters. TACI-Fc material purified as described in Example 1 was prepared to achieve a final concentration of 158-193 mg/mL, and the TACI-Fc was finally prepared as described in Example 2.

Formulations were prepared as detailed in Example 2.A. Formulations were filled to a volume of 0.5 mL in a 1 mL NEOPAK™ syringe (from BD, product number 47433010) with HYPAK™ 4023 Flurotec stopper (from BD, product number 47375910).

### A. Characterization of TACI-Fc in Different Formulations

The properties of the TACI-Fc in the various formulations were assessed to determine the impact of the formulation components, the amounts of the components, pH, and the amount of TACI-Fc on long term stability of TAC-Fc after transport stress and long-term storage.

Using the assays listed in Table E25, stability of TACI-Fc formulated in different formulation buffers at one of four pH values (pH 4.3, 4.4, 4.6 and 5.0), one of three amounts of Arg-HCl (2.79%, 3.1%, and 3.41% w/v), and one of three tested poloxamer 188 amounts (0.04%, 0.16% and 0.1% w/v) was assessed. As set forth in Table E25, TACI-Fc stability was assessed by SEC, rCE-SDS, icIEF, subvisible particle formation, and by visual inspection after storage at −70° C., 5° C., 25° C., or 40° C. All samples underwent transportation stress as described in example 6 prior to incubation for up to 12 weeks, at the indicated temperatures.

Stability was assessed after storage using the assays summarized in Table E25.

TABLE E25
Assays for Assessing TACI-Fc Stability

| Designator | Assay |
|---|---|
| A | Color, clarity, visible particles, SEC, rCE-SDS, |
| | icIEF |
| B | Potency against APRIL and BAFF by ELISA and cell |
| | based assay |
| C | pH |
| D | Protein concentration |
| E | Subvisible particles (HIAC) |
| F | Osmolality |
| G | MAM |

A summary of the stability assays, storage temperature, and storage times assessed in this study is set forth in Table E26.

TABLE E26
Conditions Tested and Assays for Each Condition and Timepoint

| | Temperature | | | |
|---|---|---|---|---|
| | | | 25° C./60% | 40° C./75% |
| | | | relative | relative |
| Time | −70° C. | 5° C. | humidity (RH) | humidity (RH) |
| 2 weeks | ABEG | ABEG | ABEG | ABEG |
| 4 weeks | ABEG | ABEG | ABEG | ABEG |
| 8 weeks | ABEG | ABEG | ABEG | ABEG |
| 12 weeks | ABEG | ABEG | ABEG | ABCDEG |

### 1. Size Exclusion Chromatography (SEC)

SEC was used to assess stability as defined by the rate of loss in percent main species of the TACI-Fc protein in the various formulations after incubation for 12 weeks at the various temperatures as detailed in Example 2. Rate was calculated by linear regression of the data using the method of least squares fit. Error bars are the calculated standard error in the rate calculation.

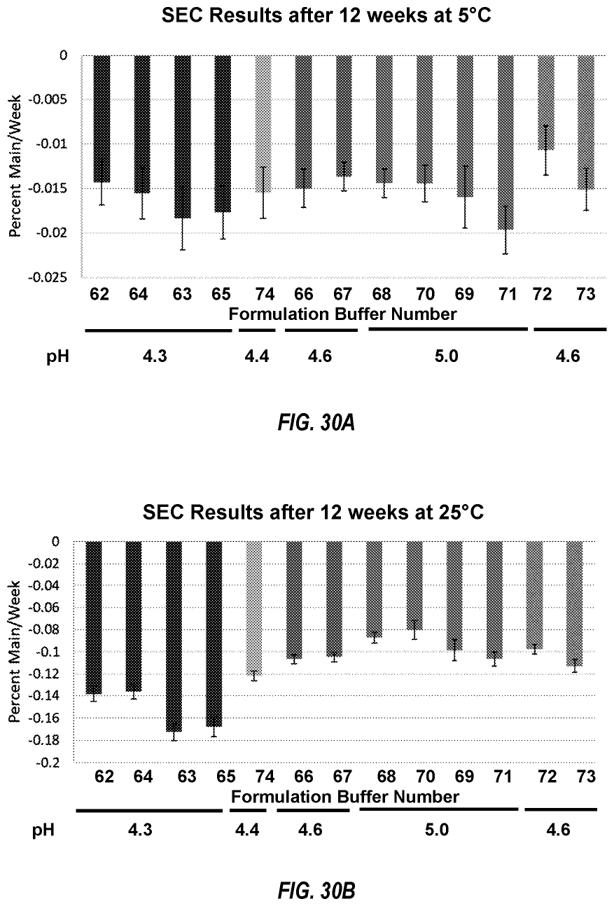

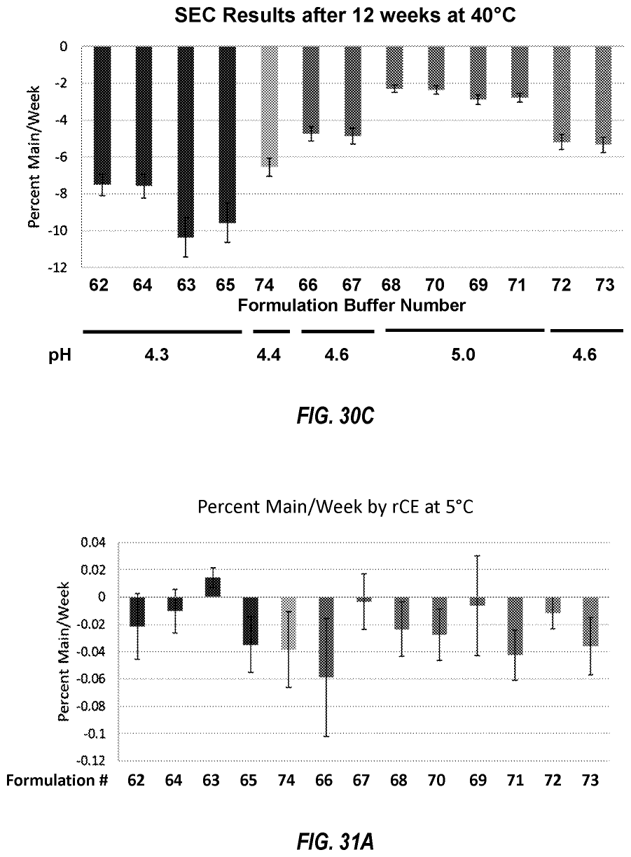

In this study, the SEC results show that after 12 weeks of storage at 5° C., there was less than up to 0.02%/week loss of the TACI-Fc main species for all formulation buffers. Differences between formulation buffers were more apparent after incubation at higher temperatures of 25° C. and 40° C. The results show that there was a higher decrease in the percent main of the TACI-Fc for formulations at the lowest pH of 4.3 (formulations 62-65) incubated for 12 weeks, as shown in FIG. 30B (at 25° C.) and FIG. 30C (at 40° C.). Formulations 66, 67, 72, and 73, which were formulated at pH 4.6, and Formulation 75, formulated at pH 4.4, showed lower loss of the main TACI-Fc species over time compared to formulations at pH 4.3. Formulations at pH 5.0 also showed good stability with decreased loss of the main TACI-Fc species. The loss of TACI-Fc main species per week after storage at 5° C. was approximately 0.014%-0.015% per week for most conditions, which equates to a loss of approximately 0.7%-0.8% after 1 year. These results show that after 12 weeks the TACI-Fc fusion protein is less stable in formulations at the lowest pH 4.3 when assessed by SEC.

### 2. Reduced Capillary Electrophoresis Sodium Dodecyl Sulfate (rCE-SDS)

Using rCE-SDS assays as detailed in Example 5, the percent decrease in the main TACI-Fc species was assessed after preparation in the 13 formulation buffers in Table E24 after storage at 5° C., 25° C. or 40° C. for 12 weeks. Data was collected at 5 times points over the 12-week period and the rate of the loss of the main TACI-Fc species was calculated per week over the 12 week period. The rate was calculated assuming a linear relationship and error bars represent the standard error in the rate calculation.

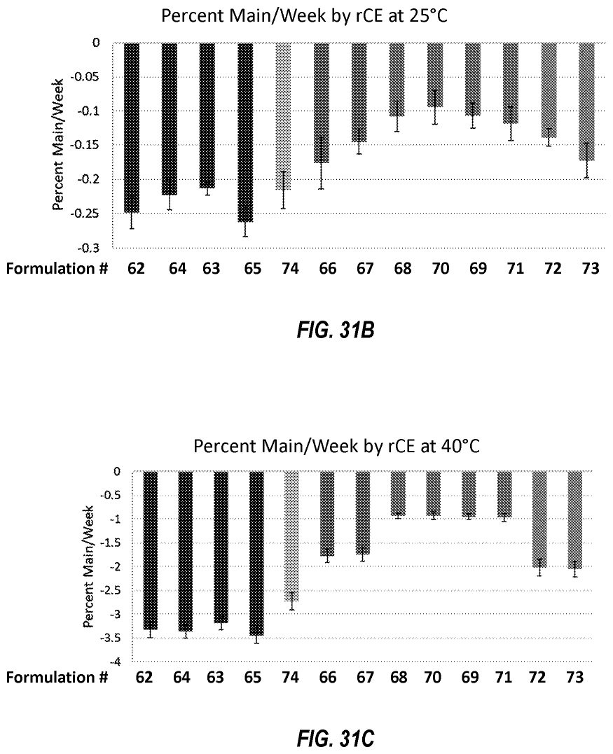

In this study, the rCE-SDS results were similar to the SEC results. The rCE-SDS results show that after 12-week storage at 5° C., there was less than about 0.06%/week loss of the TACI-Fc main species for all formulation buffers, as shown in FIG. 31A. Differences between formulation buffers were more apparent after incubation at higher temperatures of 25° C. and 40° C., with the most loss seen after incubation at 40° C. The results show that there was generally a higher decrease in the percent main of the TACI-Fc for formulations at the lowest pH of 4.3 (formulations 62-65) incubated for 12 weeks, as shown in FIG. 31B (at 25° C.), and FIG. 31C (at 40° C.). Formulations 66, 67, 72, and 73, which were formulated at pH 4.6 and Formulation 75, formulated at pH 4.4 showed lower loss of the main TACI-Fc species over time at 40° C. Formulations at pH 5.0 also showed good stability with a decreased loss of the main TACI-Fc species compared to formulations at pH 4.1. The loss of TACI-Fc main (homodimer) species per week after storage at 5° C. was approximately 0.003%-0.06% per week for most conditions, which equates to a loss of approximately 0.2%-3.1% after 1 year. These results show that after 12 weeks, the TACI-Fc fusion protein is less stable in formulations at the lowest pH of 4.3 when assessed by rCE-SDS.

### 3. Imaged Capillary Isoelectric Focusing (icIEF)

Using icIEF assays as detailed in Example 5, the percent decrease in the main TACI-Fc was assessed after preparation in the 13 formulation buffers in Table E24 after storage at 5° C., 25° C. or 40° C. for 12 weeks. Data was collected at 5 times points over the 12-week period and the rate of the loss of the main TACI-Fc species was calculated per week over the 12-week period. Rate was calculated by linear regression of the data using the method of least squares fit. Error bars were the calculated standard error in the rate calculation.

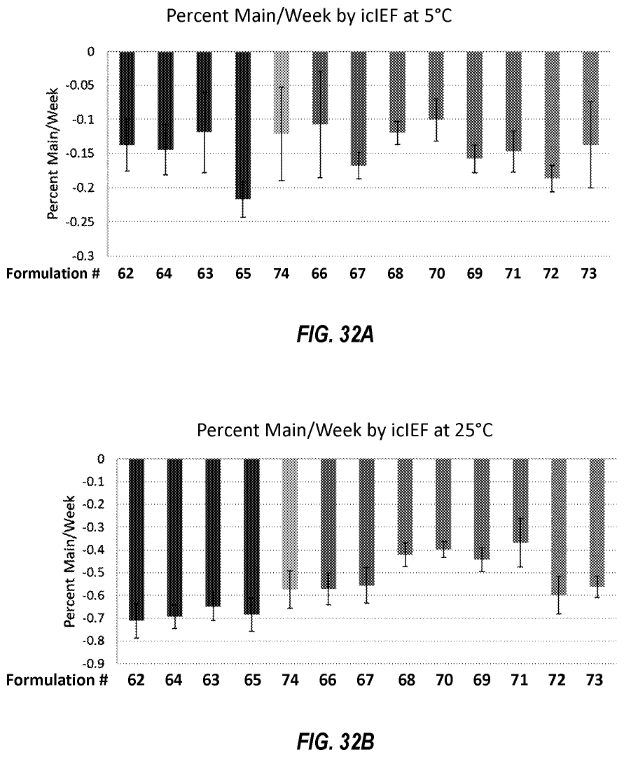

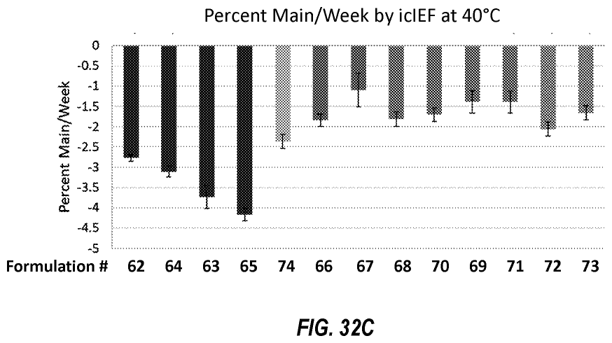

In this study, the icIEF main species results trended similarly with the rCE-SDS and SEC main species results. The icIEF results show that after 12 weeks of storage at 5° C., there was less than about 0.2%/week loss of the TACI-Fc main species for all formulation buffers, as shown in FIG. 32A. Differences between formulation buffers were more apparent after incubation at higher temperatures of 25° C. and 40° C., with the greatest loss of TACI-Fc main species seen after incubation at 40° C. The results show that there was a greater decrease in the percent main species of the TACI-Fc for formulations at the lowest pH of 4.3 (formulations 62-65) incubated for 12 weeks, as shown in FIG. 32B (at 25°), and FIG. 32C (at 40°). Formulations prepared at pH at or above 4.6 showed a lower loss of the TACI-Fc main species over time and were stable in all of formulations 66-74. Formulations 66, 67, 72, and 73, which were formulated at pH 4.6, showed less loss of the main TACI-Fc species over time compared to formulations at pH 4.3. Formulations at pH 5.0 also showed good stability with decreased loss of the main TACI-Fc species. These results show that after 12 weeks, the TACI-Fc fusion protein is less stable in formulations at the lowest pH of 4.3 when assessed by icIEF, and formulations prepared between pH 4.6 and 5.0 showed good stability of the TACI-Fc main species.

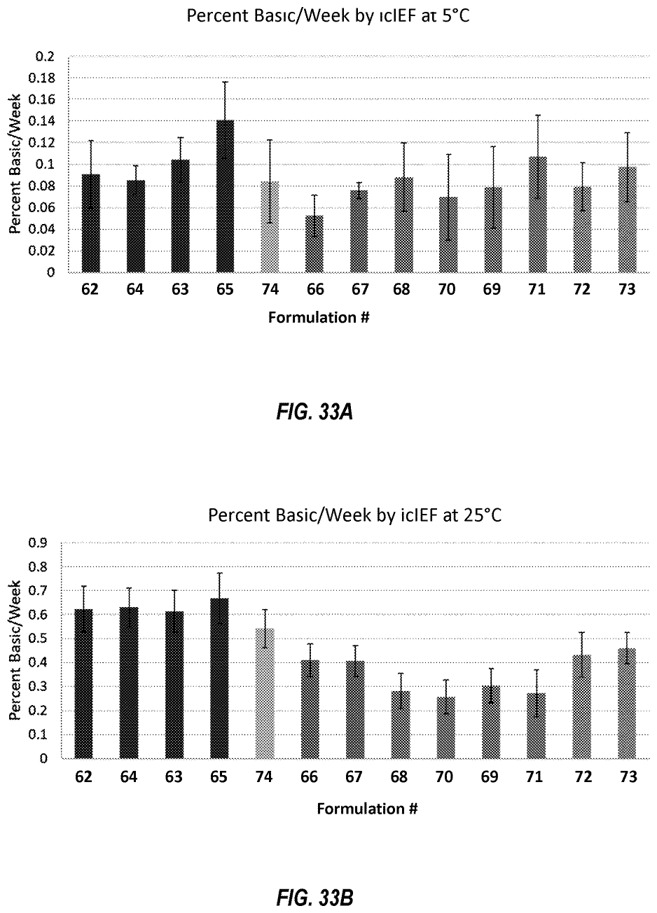

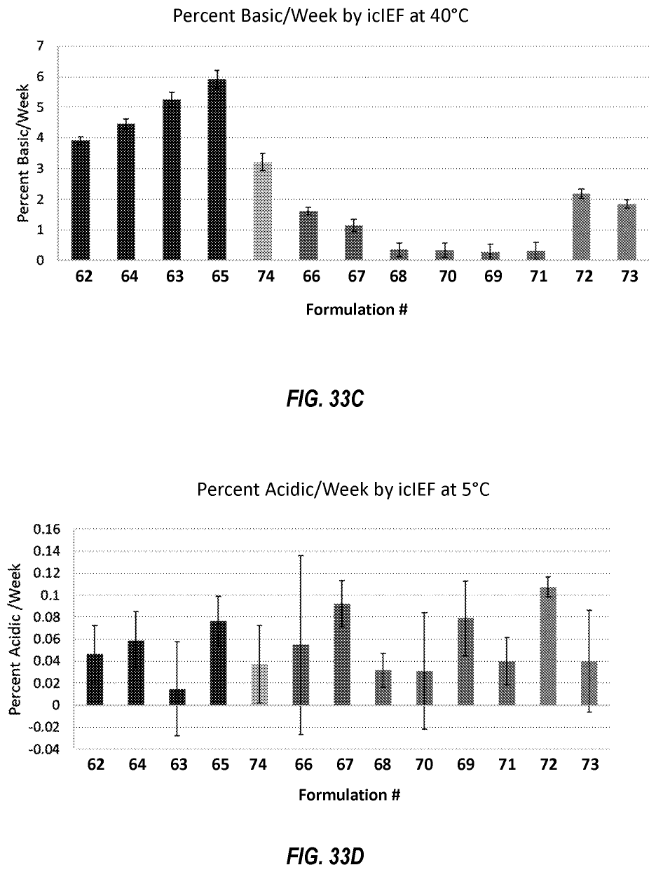

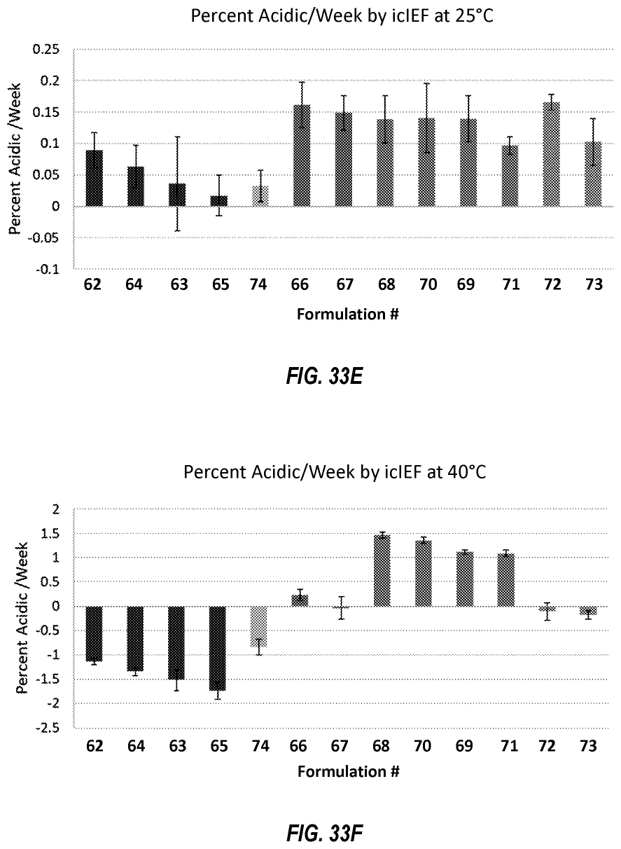

The charge species of the TACI-Fc also was assessed by imaged capillary isoelectric focusing (icIEF) as described in Example 5. The total percent of basic and acidic species formed per week were assessed over the 12-week time period after incubation of the TACI-Fc in the 13 formulation buffers at 5° C., 25° C. and 40° C. The results show a shift to the basic (% basic) species after incubation at elevated temperatures of 40° C. as shown in FIG. 33C. The shift was pH-dependent, as the samples having a lower pH shifted more basic after incubation at 40° C. A concomitant decrease in the percent acidic (% acidic) species of the TACI-Fc after storage for 12 weeks at 40° C. was also noted for the low pH (pH 4.3) formulations, as shown in FIG. 33F.

### 4. Subvisible Particle Assessment

A HIAC Liquid Particle Counter (Beckman Coulter) and FlowCam® particle analyzer (Yokogawa) were used to measure the number of subvisible particles for samples after storage. Assessments were conducted in accordance with the manufacturer's instructions to count the subvisible particles in the samples. Assessment using the HIAC Liquid Particle Counter was conducted as described in Example 5. USP guidelines specify no more than 6,000 ≥10 μm particles per container; and no more than 600 ≥25 μm particles/container.

The cumulative number of particles ≥2 μm, ≥10 μm, or ≥25 μm per container, was counted using a HIAC Liquid Particle Counter (Beckman Coulter) after storage of the 13 formulations containing the TACI-Fc for 12 weeks at 5° C., 25° C. and 40° C. The results show that for all formulations at 5° C. and 25° C., the number of subvisible particles particle counts were within USP guidelines for all conditions tested.

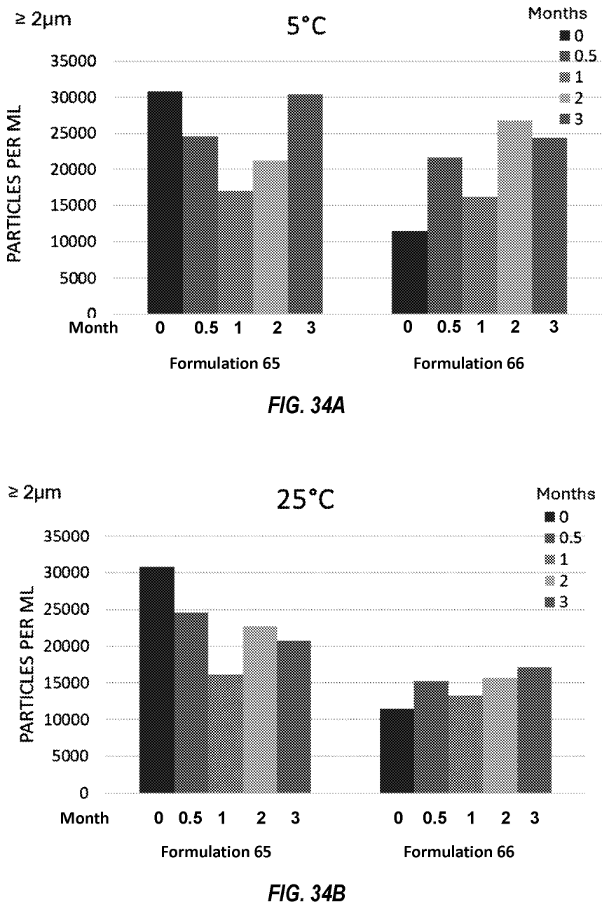

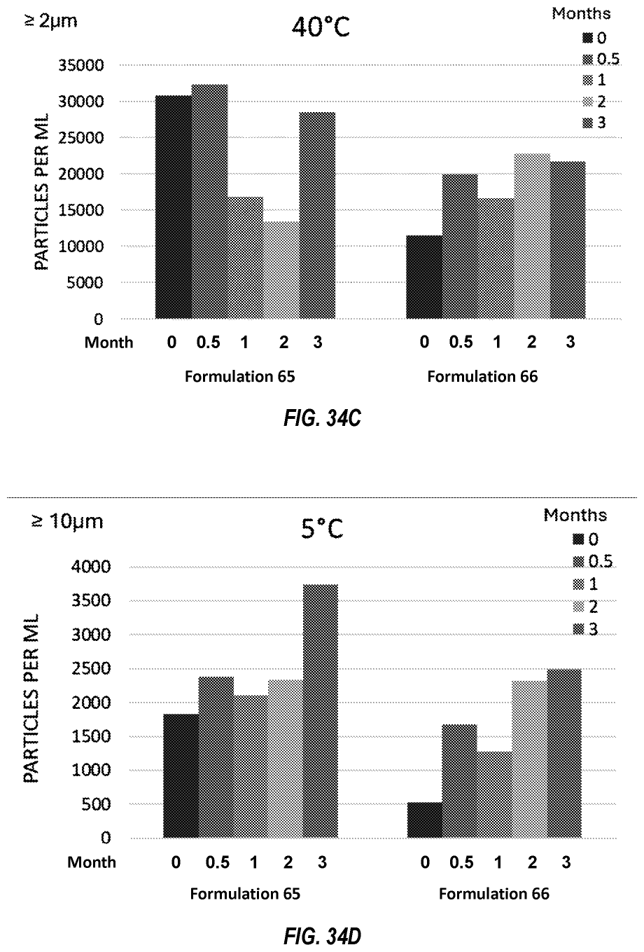

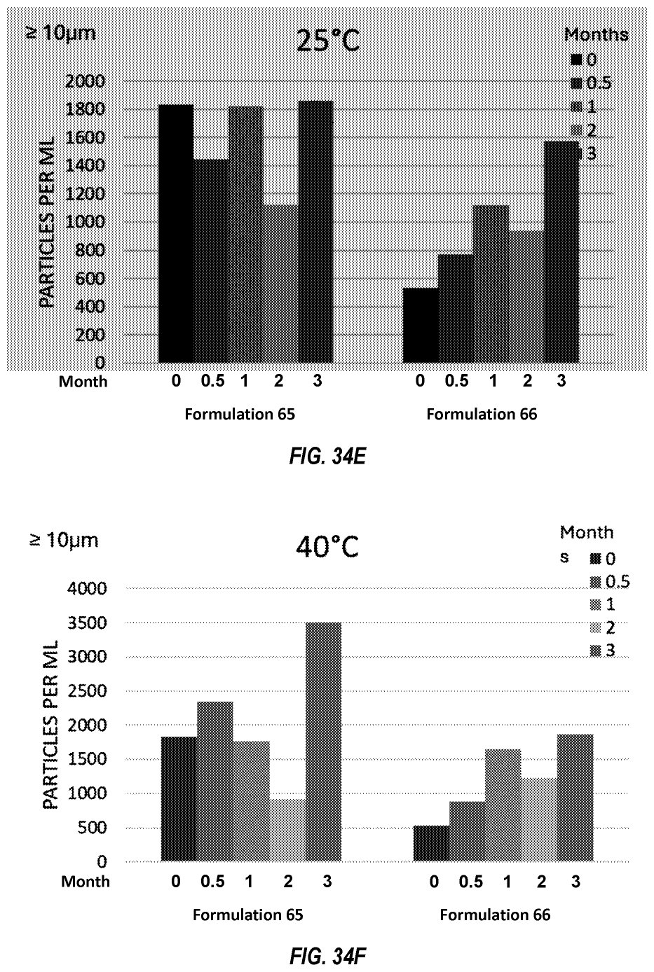

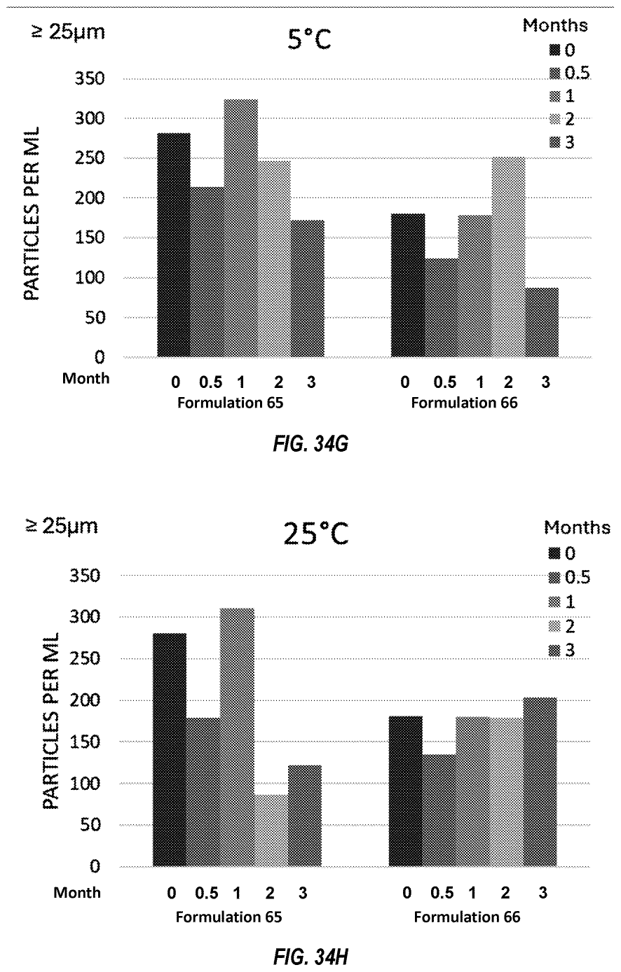

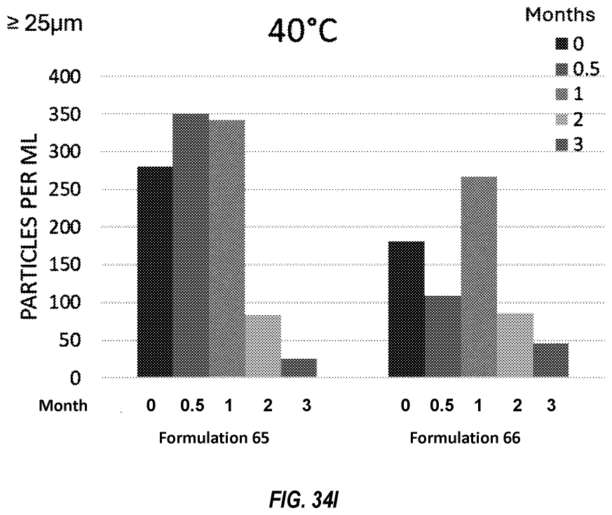

The cumulative number of particles ≥2 μm, ≥10 μm, or ≥25 μm per container, was also counted using a FlowCam® particle analyzer (Yokogawa) after storage of Formulation 66 and Formulation 67 containing the 175 mg/mL TACI-Fc formulate with 3.1% Arg-HCl (w/v) and 0.1% P188 (w/v) at pH 4.6 for 12 weeks at 5° C., 25° C. and 40C°. The results show that the number of subvisible particles particle counts in Formulations 66 and 67 were comparable. The highest number of particles ≥10 μm was after incubation for 3 months at 5° C. FIGS. 34A-I show the particle counts as assessed by FlowCam® particle analyzer for Formulations 66 and 67, containing 10 mM sodium acetate, 3.1% arginine HCl (w/v), 0.1% poloxamer 188 w/v and 175 mg/mL TACI-Fc formulated at pH 4.6. The results show that all TACI-Fc formulations particle counts did not grow significantly over time, signifying the formulation was stable for the 12 weeks studied.

### 5. Visual Inspection

Stability of the TACI-Fc also was assessed by visual inspection after prolonged storage of the 13 formulations at different temperatures. The 13 formulations were assessed by eye for color, clarity, and the presence of visible particles after storage at 5° C., 25° C., and 40° C. for 12 weeks as described in Example 5.

All formulations showed a color of ≤BY6 after storage at 5° C. and 25° C. for up to 12 weeks. The formulations at pH ≤4.4 (formulations 62-65 and 74) and stored at 40° C. for 12 weeks converted from liquid to a gel form, and the color could not be measured after gelation. Formulations prepared at pH >4.4 (Formulations 66-73) darkened in color to <BY4 after 12 weeks at 40° C. for all samples except for formulations 68 and 70, containing 158 mg/mL TACI-Fc at pH 5.0 and with 0.04% and 0.16% poloxamer 188 w/v, respectively, which only darkened to <BY5.

The relative clarity of the prepared TACI-Fc formulations was visually inspected. All formulations showed a clarity of ≤RSII after storage at 5° C. and 25° C. for up to 12 weeks. The formulations at pH <4.4 (formulations 62-65 and 74) that were stored at 40° C. for 12 weeks converted from a liquid to a gel form, and the clarity could not be measured after gelation. Formulations at pH 4.6 and 5.0 incubated at 40° C. for 12 weeks remained in liquid form albeit the formulations at pH 4.6 were beginning to show an increase in viscosity. After 12 weeks at 40° C., formulations 66, 67, 72, and 73, which were formulated at pH 4.6 with 0.1% poloxamer 188 w/v, had an increase in clarity to <RIV, and formulations 68-71, which were formulated at pH 5.0 with either 0.04% or 0.16% poloxamer 188 w/v, increased in clarity to <RIII.

The presence of visible particles in the prepared TACI-Fc formulations was assessed by visible inspection. The formulations were essentially free of visible particles for all time points and temperatures studies. A single fiber or particle was rarely observed.

As noted above, a subset of the formulations had gelled after incubation at 40° C. for 12 weeks. When assessing the syringeability of the formulations, there was variation in the viscosity of the formulations. Formulations at pH 4.3 and pH 4.4 (formulations 62-65 and 74) were sufficiently viscous that it was difficult or not possible to fully expel them from the prefilled syringe due to their gelled state, and any bubbles that formed in the syringe could not be moved. After storage at 40° C. for 12 weeks, formulations at pH 4.6 (formulations 66, 67, 72 and 73) were visually assessed and determined to be somewhat more viscous than the corresponding formations stored at 25° C., but the pH 4.6 formulations were still liquid, could be taken up and expelled by a syringe, and any bubbles in the formulation were movable. All samples stored at 25° C. for 12 weeks were liquid. These results show that storage at 40° C. for 12 weeks can lead to consistency changes of the TACI-Fc formulation, and the lowest pH levels (≤4.4) showed the most changes in consistency and were in a gelled form after prolonged exposure to 40° C.

After assessment by visual inspection, all of the formulations were determined to be satisfactory after incubation at 5° C. or 25° C. for 12 weeks. After storage at 40° C. for 12 weeks, samples at pH ≤4.4 had gelled, only some formulations could be fully expelled from the syringe, and their color and clarity could not be measured.

### 6. TACI-Fc Anti-APRIL and Anti-BAFF Activity

Activity of the TACI-Fc for APRIL and BAFF also was assessed formulations 66 and 67 as detailed in Example 6. Additional analysis of activity by cell-based assay was also performed. Cell-based assay test samples were diluted and plated with Jurkat NF-κB reporter cells transduced with TACI. TACI-Fc inhibited activation of TACI by APRIL or BAFF in a TACI-Fc dose-dependent manner. Relative potency was determined through comparison of dose-response midpoint concentrations against a reference standard. The bioactivity of the TACI-Fc protein in formulation buffers 66 and 67 stored at −70° C. in vials and in syringes at 4° C., 25° C., and 40° C. for up to 12 weeks, was assessed.

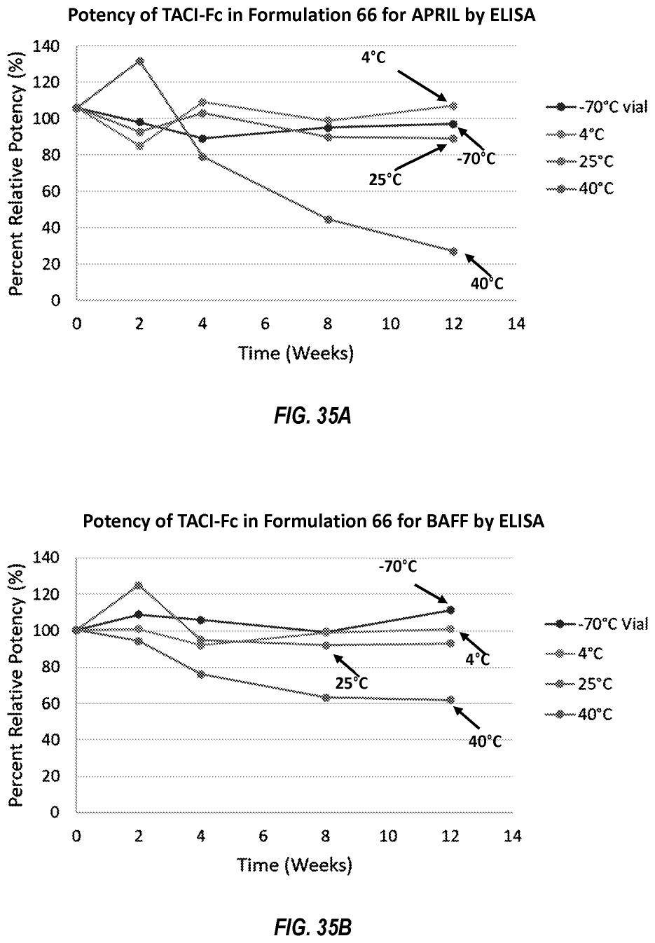

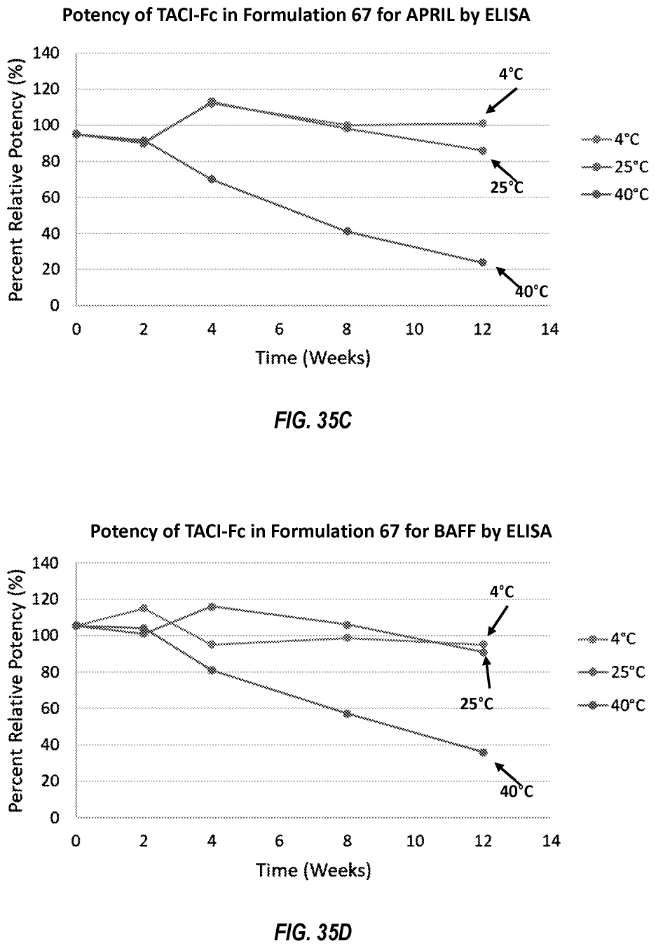

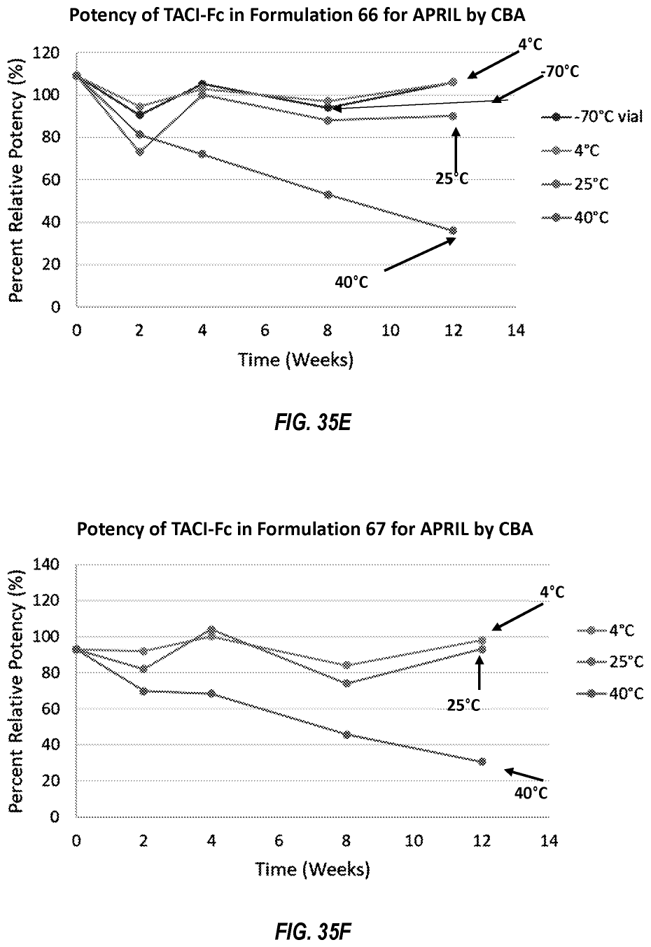

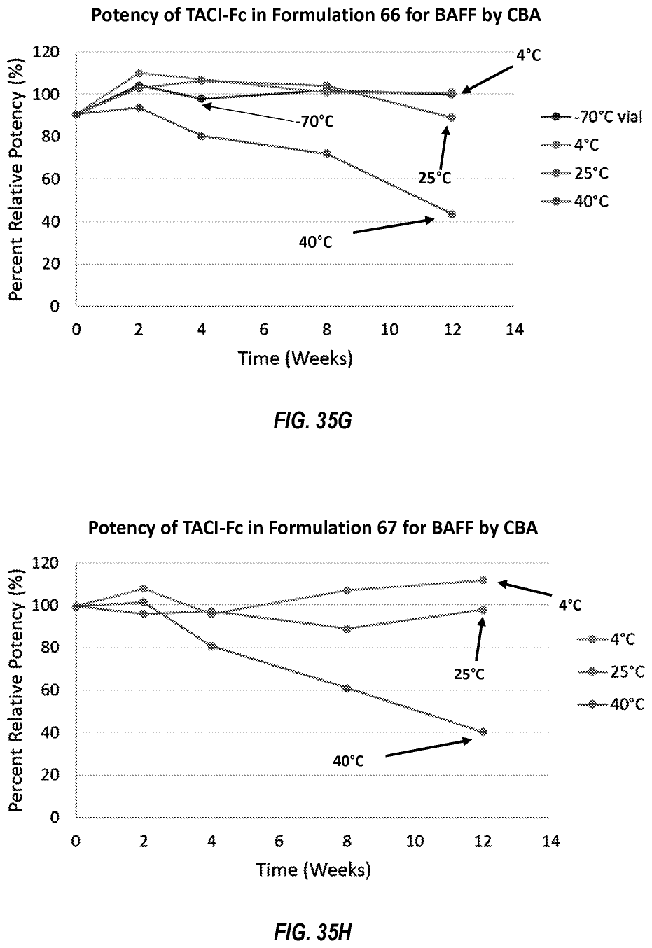

As shown in FIGS. 35A-H, TACI-Fc formulated in Formulations 66 and 67 retained APRIL- and BAFF-inhibitory activity for up to 12 weeks, when stored at −70° C., 4° C. and 25° C. After prolonged storage at 40° C. for 12 weeks, the TACI-Fc formulated in buffers 66 and 67 showed a decreased activity for both APRIL and BAFF, such that after 8-12 weeks, the amount of activity for APRIL or BAFF was outside the qualified range. These results show that TACI-Fc stored in formulation buffers 66 and 67 can maintain activity for APRIL and BAFF after prolonged storage at −70° C., 4° C. and 25° C., but did not retain similar activity after prolonged storage at the higher temperature of 40° C.

### Summary

The results in Example 8 show that TACI-Fc formulated at concentrations from 158 mg/mL to 193 mg/mL in a buffer containing arginine HCl and poloxamer 188 at pH ≥4.4 is stable after incubation for up to 12 weeks at temperatures between −70° C. and 25° C., by SEC, rCE and icIEF, and was within the USP levels by light obscuration for particles in a solution for administration. These formulations also did not change in their visible characteristics (e.g., color and clarity) and retained a liquid form that flowed and could be pulled up and expelled by a needle after prolonged storage. Formulation buffers containing any of 3.79%, 3.1%, or 3.41% Arg-HCl w/v and 0.04%, 0.16% or 0.1% of the surfactant poloxamer 188 w/v at pH at or above 4.4 (e.g., 4.6 and 5.0) showed stabilizing effects on the TACI-Fc in the formulation. These results indicate that TACI-Fc can be formulated at a high protein concentrations of 158-198 mg/mL in buffers containing the identified components at a range of concentrations and maintain stability and activity.

### Example 9

### Summary

Examples 1-8 assessed the properties and stability of a TACI-Fc fusion protein in a variety of formulation buffers containing different formulation components, different amounts of the components, and different surfactants, and where the formulations were prepared at different pH values. The properties and stability of the TACI-Fc fusion protein in the formulations also were assessed after long-term storage at various temperatures, from freezing to warm, and when subjected to simulated transport, including temperature, drops and vibrations.

Initial experiments in Example 2 showed that the formulation pH impacted TACI-Fc stability. The results showed viscosity increase for some formulations with increasing pH, and, by SEC, there was a pH-dependent decrease in percent main species of TACI-Fc (homodimer) in acetate formulations, where lower-pH formulations showed a lower decrease in percent main species (i.e., more TACI-Fc fusion protein was maintained in the homodimer state). By SEC, after storage at 2-8° C., the acetate formulations at pH 5.06 and pH 5.21 were the most stabilizing, and after storage at 30° C., the acetate formulations at pH 5.06 showed the least decrease in % main species (homodimer). The SEC results also showed that after incubation at 30° C., the presence of sodium chloride improves stability in low pH (5.06) conditions, where there was less decrease in the percent main species in formulations containing NaCl compared to those without. When the subvisible particles in the formulations were observed, the results by BMI showed high particle concentrations and large black particles (≥25 μm) with NaCl concentrations above 50 mM after 8 weeks at 4° C. In view of these results, further experiments were conducted to assess whether modifications to the formulations identified in Example 2 can further improve TACI-Fc stability, including long term stability at different temperatures and when subjected to simulated transport.

Because TACI-Fc formulations at the lowest pH of 5.06 tested in Example 2 showed increased TACI-Fc homodimer stability, Example 3 assessed even lower pH formulations, and also assessed many different formulation components to determine their impact on TACI-Fc homodimer stability. The results from Example 3 showed that lowering the pH to 4.5 increased TACI-Fc stability in arginine-containing formulations. By SEC, the results in Example 3 had similar trends to Example 2, and showed a pH-dependent decrease in TACI-Fc percent main species upon storage in acetate-arginine formulations, where low pH resulted in less decrease in percent main species. In formulations tested in Example 3, isotonic formulations prepared at pH 4.5 had the least decrease in % main species compared to the higher pH of 5.1 and 5.5. By SEC, after incubation at 2-8° C. or 30° C., a formulation containing Arg-HCl formulated at a pH of 4.5 (Formulation 22) showed much lower formation of HMW TACI-Fc species and lower loss of the % main species compared to Formulation 39 (at pH 5.1), which was similar to a stable formulation identified in Example 2. The results in Example 3 showed that the TACI-Fc homodimer is stable in a formulation containing 3.1% Arg-HCl and that the stability is pH-dependent.

The impact of modifying the surfactant concentration, or modifying the amounts of all components was assessed in Examples 6 and 8. In Example 6, five different poloxamer 188 concentrations were tested, and in Example 8, formulations at one of four pH values (pH 4.3, 4.4, 4.6 and 5.0), one of three amounts of Arg-HCl (2.79%, 3.1%, and 3.41% w/v), and one of three tested poloxamer 188 amounts (0.04%, 0.16% and 0.100 w/v) were tested. All of these formulations in Example 8 were subjected to long-term storage at different temperatures. These experiments identified formulation conditions where the TAC-Fc homodimer showed long term stability (low loss of the “main” TACI-Fc species) for extended periods at different temperatures.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCE TABLE

| SEQ ID NO. | Description | Amino Acid or Nucleotide Sequence |
|---|---|---|
| SEQ ID NO: 1 | TACI WT ECD | VAMRSCPEEQYWDPLLGTCMSCKTICNHQSQRTCAAFC |
| | | RSLSCRKEQGKFYDHLLRDCISCASICGQHPKQCAYFCEN |
| | | KLRS |
| SEQ ID NO: 2 | L82P | VAMRSCPEEQYWDPLLGTCMSCKTICNHQSQRTCAAFC |
| | | RSLSCRKEQGKFYDHPLRDCISCASICGQHPKQCAYFCEN |
| | | KLRS |
| SEQ ID NO: 3 | D85E, K98T | VAMRSCPEEQYWDPLLGTCMSCKTICNHQSQRTCAAFC |
| | | RSLSCRKEQGKFYDHLLRECISCASICGQHPTQCAYFCEN |
| | | KLRS |
| SEQ ID NO: 4 | I87L, K98T | VAMRSCPEEQYWDPLLGTCMSCKTICNHQSQRTCAAFC |
| | | RSLSCRKEQGKFYDHLLRDCLSCASICGQHPTQCAYFCE |
| | | NKLRS |
| SEQ ID NO: 5 | R60G, Q75E, L82P | VAMRSCPEEQYWDPLLGTCMSCKTICNHQSQGTCAAFC |
| | | RSLSCRKEEGKFYDHPLRDCISCASICGQHPKQCAYFCEN |
| | | KLRS |
| SEQ ID NO: 6 | R60G, C86Y | VAMRSCPEEQYWDPLLGTCMSCKTICNHQSQGTCAAFC |
| | | RSLSCRKEQGKFYDHLLRDYISCASICGQHPKQCAYFCE |
| | | NKLRS |
| SEQ ID NO: 7 | A101D | VAMRSCPEEQYWDPLLGTCMSCKTICNHQSQRTCAAFC |
| | | RSLSCRKEQGKFYDHLLRDCISCASICGQHPKQCDYFCEN |
| | | KLRS |
| SEQ ID NO: 8 | C86Y | VAMRSCPEEQYWDPLLGTCMSCKTICNHQSQRTCAAFC |
| | | RSLSCRKEQGKFYDHLLRDYISCASICGQHPKQCAYFCE |
| | | NKLRS |
| SEQ ID NO: 9 | W40R, L82P, | VAMRSCPEEQYRDPLLGTCMSCKTICNHQSQRTCAAFCR |
| | F103Y | SLSCRKEQGKFYDHPLRDCISCASICGQHPKQCAYYCEN |
| | | KLRS |
| SEQ ID NO: 10 | W40R, Q59R, | VAMRSCPEEQYRDPLLGTCMSCKTICNHQSRRPCAAFCR |
| | T61P, K98T | SLSCRKEQGKFYDHLLRDCISCASICGQHPTQCAYFCENK |
| | | LRS |
| SEQ ID NO: 11 | L82P, I87L | VAMRSCPEEQYWDPLLGTCMSCKTICNHQSQRTCAAFC |
| | | RSLSCRKEQGKFYDHPLRDCLSCASICGQHPKQCAYFCE |
| | | NKLRS |
| SEQ ID NO: 12 | G76S, P97S | VAMRSCPEEQYWDPLLGTCMSCKTICNHQSQRTCAAFC |
| | | RSLSCRKEQSKFYDHLLRDCISCASICGQHSKQCAYFCEN |
| | | KLRS |
| SEQ ID NO: 13 | TACI WT ECD | SLSCRKEQGKFYDHLLRDCISCASICGQHPKQCAYFCEN |
| | | KLRS |
| SEQ ID NO: 14 | D85V | SLSCRKEQGKFYDHLLRVCISCASICGQHPKQCAYFCEN |
| | | KLRS |
| SEQ ID NO: 15 | E74V | SLSCRKVQGKFYDHLLRDCISCASICGQHPKQCAYFCEN |
| | | KLRS |
| SEQ ID NO: 16 | R84L | SLSCRKEQGKFYDHLLLDCISCASICGQHPKQCAYFCENK |
| | | LRS |
| SEQ ID NO: 17 | K77E, R84L, | SLSCRKEQGEFYDHLLLDCISCASICGQHPKQCAYYCEN |
| | F103Y | KLRS |
| SEQ ID NO: 18 | Y79F, Q99E | SLSCRKEQGKFFDHLLRDCISCASICGQHPKECAYFCENK |
| | | LRS |
| SEQ ID NO: 19 | Y79F | SLSCRKEQGKFFDHLLRDCISCASICGQHPKQCAYFCENK |
| | | LRS |
| SEQ ID NO: 20 | R84G | SLSCRKEQGKFYDHLLGDCISCASICGQHPKQCAYFCEN |
| | | KLRS |
| SEQ ID NO: 21 | L83S, F103S | VAMRSCPEEQYWDPLLGTCMSCKTICNHQSQRTCAAFC |
| | | RSLSCRKEQGKFYDHLSRDCISCASICGQHPKQCAYSCEN |
| | | KLRS |
| SEQ ID NO: 22 | L82H | VAMRSCPEEQYWDPLLGTCMSCKTICNHQSQRTCAAFC |
| | | RSLSCRKEQGKFYDHHLRDCISCASICGQHPKQCAYFCE |
| | | NKLRS |
| SEQ ID NO: 23 | A101D | SLSCRKEQGKFYDHLLRDCISCASICGQHPKQCDYFCEN |
| | | KLRS |
| SEQ ID NO: 24 | K77E, R84Q | SLSCRKEQGEFYDHLLQDCISCASICGQHPKQCAYFCENK |
| | | LRS |
| SEQ ID NO: 25 | K77E, A101D | SLSCRKEQGEFYDHLLRDCISCASICGQHPKQCDYFCENK |
| | | LRS |
| SEQ ID NO: 26 | K77E, F78Y, | SLSCRKEQGEYYDHLLRDCISCASICGQHPKQCADFCEN |
| | Y102D | KLRS |
| SEQ ID NO: 27 | Q75E, R84Q | SLSCRKEEGKFYDHLLQDCISCASICGQHPKQCAYFCENK |
| | | LRS |
| SEQ ID NO: 28 | Q75R, R84G, I92V | SLSCRKERGKFYDHLLGDCISCASVCGQHPKQCAYFCEN |
| | | KLRS |
| SEQ ID NO: 29 | K77E, A101D, | SLSCRKEQGEFYDHLLRDCISCASICGQHPKQCDDFCENK |
| | Y102D | LRS |
| SEQ ID NO: 30 | R84Q | SLSCRKEQGKFYDHLLQDCISCASICGQHPKQCAYFCEN |
| | | KLRS |
| SEQ ID NO: 31 | R84Q, S88N, | SLSCRKEQGKFYDHLLQDCINCASICGQHPKQCDYFCEN |
| | A101D | KLRS |
| SEQ ID NO: 32 | K77E | SLSCRKEQGEFYDHLLRDCISCASICGQHPKQCAYFCENK |
| | | LRS |
| SEQ ID NO: 33 | R84Q, F103V | SLSCRKEQGKFYDHLLQDCISCASICGQHPKQCAYVCEN |
| | | KLRS |
| SEQ ID NO: 34 | K77E, Q95R, | SLSCRKEQGEFYDHLLRDCISCASICGRHPKQCDYFCENK |
| | A101D | LRS |
| SEQ ID NO: 35 | I87M, A101D | SLSCRKEQGKFYDHLLRDCMSCASICGQHPKQCDYFCEN |
| | | KLRS |
| SEQ ID NO: 36 | TACI WT ECD | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTTGCGAGACTGTATAAGCTGTGCGTC |
| | | TATTTGTGGACAACACCCTAAACAATGTGCTTATTTCT |
| | | GTGAAAATAAGCTTCGATCT |
| SEQ ID NO: 37 | L82P | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCCTTTGCGAGACTGTATAAGCTGTGCGTC |
| | | TATTTGTGGACAACACCCTAAACAATGTGCTTATTTCT |
| | | GTGAAAATAAGCTTCGATCT |
| SEQ ID NO: 38 | D85E, K98T | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTTGCGAGAATGTATAAGCTGTGCGTC |
| | | TATTTGTGGACAACACCCTACACAATGTGCTTATTTCT |
| | | GTGAAAATAAGCTTCGATCT |
| SEQ ID NO: 39 | I87L, K98T | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTTGCGAGACTGTTTAAGCTGTGCGTC |
| | | TATTTGTGGACAACACCCTACACAATGTGCTTATTTCT |
| | | GTGAAAATAAGCTTCGATCT |
| SEQ ID NO: 40 | R60G, Q75E, L82P | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAGGGACATGCGCAGCTTTCTG |
| | | CAGGAGTCTCTCTTGCAGGAAAGAGGAAGGCAAATTT |
| | | TACGACCATCCTTTGCGAGACTGTATAAGCTGTGCGTC |
| | | TATTTGTGGACAACACCCTAAACAATGTGCTTATTTCT |
| | | GTGAAAATAAGCTTCGATCT |
| SEQ ID NO: 41 | R60G, C86Y | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAGGGACATGCGCAGCTTTCTG |
| | | CAGGAGTCTCTCTTGCAGGAAAGAGCAAGGCAAATTT |
| | | TACGACCATCTTTTGCGAGACTACATAAGCTGTGCGTC |
| | | TATTTGTGGACAACACCCTAAACAATGTGCTTATTTCT |
| | | GTGAAAATAAGCTTCGATCT |
| SEQ ID NO: 42 | A101D | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTTGCGAGACTGTATAAGCTGTGCGTC |
| | | TATTTGTGGACAACACCCTAAACAATGTGATTATTTCT |
| | | GTGAAAATAAGCTTCGATCT |
| SEQ ID NO: 43 | C86Y | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTTGCGAGACTATATAAGCTGTGCGTC |
| | | TATTTGTGGACAACACCCTAAACAATGTGCTTATTTCT |
| | | GTGAAAATAAGCTTCGATCT |
| SEQ ID NO: 44 | W40R, L82P, | GTGGCAATGCGCTCATGCCCAGAGGAACAATATCGGG |
| | F103Y | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCCTTTGCGAGACTGTATAAGCTGTGCGTC |
| | | TATTTGTGGTCAACACCCTAAACAATGTGCTTATTACT |
| | | GTGAAAATAAGCTTCGATCT |
| SEQ ID NO: 45 | W40R, Q59R, | GTGGCAATGCGCTCATGCCCAGAGGAACAATATCGGG |
| | T61P, K98T | ATCCGCTTCTTGGGACGTGTATGAGCTGCAAGACCATC |
| | | TGTAATCATCAATCCCGAAGGCCATGCGCAGCATTCTG |
| | | CAGGAGTCTCTCTTGCAGGAAAGAGCAAGGCAAATTT |
| | | TACGACCATCTTTTGCGAGACTGTATAAGCTGTGCGTC |
| | | TATTTGTGGACAACACCCTACACAATGTGCTTATTTCT |
| | | GTGAAAATAAGCTTCGATCT |
| SEQ ID NO: 46 | L82P, I87L | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCCTTTGCGAGACTGTTTAAGCTGTGCGTC |
| | | TATTTGTGGACAACACCCTAAACAATGTGCTTATTTCT |
| | | GTGAAAATAAGCTTCGATCT |
| SEQ ID NO: 47 | G76S, P97S | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAAGCAAATTT |
| | | TACGACCATCTTTTGCGAGACTGTATAAGCTGTGCGTC |
| | | TATTTGTGGTCAACACTCTAAACAATGTGCTTATTTCT |
| | | GTGAAAATAAGCTTCGATCT |
| SEQ ID NO: 48 | Wt | AGTCTCTCTTGCAGGAAAGAGCAAGGCAAATTTTACG |
| | | ACCATCTTTTGCGAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 49 | D85V | AGTCTCTCTTGCAGGAAAGAGCAAGGCAAATTTTACG |
| | | ACCATCTTTTGCGAGTCTGTATAAGCTGTGCGTCTATT |
| | | TGTGGACAACACCCTAAACAATGTGCTTATTTCTGTGA |
| | | AAATAAGCTTCGATCT |
| SEQ ID NO: 50 | E74V | AGTCTCTCTTGCAGGAAAGTGCAAGGCAAATTTTACG |
| | | ACCATCTTTTGCGAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 51 | R84L | AGTCTCTCTTGCAGGAAAGAGCAAGGCAAATTTTACG |
| | | ACCATCTTTTGCTAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 52 | K77E, R84L, | AGTCTCTCTTGCAGGAAAGAGCAAGGCGAATTTTACG |
| | F103Y | ACCATCTTTTGCTAGACTGTATAAGCTGTGCGTCTATT |
| | | TGTGGACAACACCCTAAACAATGTGCTTATTACTGTGA |
| | | AAATAAGCTTCGATCT |
| SEQ ID NO: 53 | Y79F, Q99E | AGTCTCTCTTGCAGGAAAGAGCAAGGCAAATTTTTCG |
| | | ACCATCTTTTGCGAGACTGTATAAGCTGTGCGTCTATT |
| | | TGTGGACAACACCCTAAAGAATGTGCTTATTTCTGTGA |
| | | AAATAAGCTTCGATCT |
| SEQ ID NO: 54 | Y79F | AGTCTCTCTTGCAGGAAAGAGCAAGGCAAATTTTTCG |
| | | ACCATCTTTTGCGAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 55 | R84G | AGTCTCTCTTGCAGGAAAGAGCAAGGCAAATTTTACG |
| | | ACCATCTTTTGGGAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 56 | L83S, F103S | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTCGCGAGACTGTATAAGCTGTGCGTC |
| | | TATTTGTGGACAACACCCTAAACAATGTGCTTATTCCT |
| | | GTGAAAATAAGCTTCGATCT |
| SEQ ID NO: 57 | L82H | GTGGCAATGCGCTCATGCCCAGAGGAACAATATTGGG |
| | | ATCCGCTTCTTGGGACGTGTATGAGCTGCAAGACCATC |
| | | TGTAATCATCAATCCCAAAGGACATGCGCAGCTTTCTG |
| | | CAGGAGTCTCTCTTGCAGGAAAGAGCAAGGCAAATTT |
| | | TACGACCATCATTTGCGAGACTGTATAAGCTGTGCGTC |
| | | TATCTGTGGACAACACCCTAAACAATGTGCTTATTTCT |
| | | GTGAAAATAAGCTTCGATCT |
| SEQ ID NO: 58 | A101D | AGTCTCTCTTGCAGGAAAGAGCAAGGCAAATTTTACG |
| | | ACCATCTTTTGCGAGACTGTATAAGCTGTGCGTCTATT |
| | | TGTGGACAACACCCTAAACAATGTGATTATTTCTGTGA |
| | | AAATAAGCTTCGATCT |
| SEQ ID NO: 59 | K77E, R84Q | AGTCTCTCTTGCAGGAAAGAGCAAGGCGAATTTTACG |
| | | ACCATCTTTTGCAAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 60 | K77E, A101D | AGTCTCTCTTGCAGGAAAGAGCAAGGCGAATTTTACG |
| | | ACCATCTTTTGCGAGACTGTATAAGCTGTGCGTCTATT |
| | | TGTGGACAACACCCTAAACAATGTGATTATTTCTGTGA |
| | | AAATAAGCTTCGATCT |
| SEQ ID NO: 61 | K77E, F78Y, | AGTCTCTCTTGCAGGAAAGAGCAAGGCGAATATTACG |
| | Y102D | ACCATCTTTTGCGAGACTGTATAAGCTGTGCGTCTATT |
| | | TGTGGACAACACCCTAAACAATGTGCTGATTTCTGTGA |
| | | AAATAAGCTTCGATCT |
| SEQ ID NO: 62 | Q75E, R84Q | AGTCTCTCTTGCAGGAAAGAGGAAGGCAAATTTTACG |
| | | ACCATCTTTTGCAAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 63 | Q75R, R84G, I92V | AGTCTCTCTTGCAGGAAAGAGCGAGGCAAATTTTACG |
| | | ACCATCTTTTGGGAGACTGTATAAGCTGTGCGTCTGTT |
| | | TGTGGACAACACCCTAAACAATGTGCTTATTTCTGTGA |
| | | AAATAAGCTTCGATCT |
| SEQ ID NO: 64 | K77E, A101D, | AGTCTCTCTTGCAGGAAAGAGCAAGGCGAATTTTACG |
| | Y102D | ACCATCTTTTGCGAGACTGTATAAGCTGTGCGTCTATT |
| | | TGTGGACAACACCCTAAACAATGTGATGATTTCTGTGA |
| | | AAATAAGCTTCGATCT |
| SEQ ID NO: 65 | R84Q | AGTCTCTCTTGCAGGAAAGAGCAAGGCAAATTTTATG |
| | | ACCATCTTTTGCAAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 66 | R84Q, S88N, | AGTCTCTCTTGCAGGAAAGAGCAAGGCAAATTTTACG |
| | A101D | ACCATCTTTTGCAAGACTGTATAAACTGTGCGTCTATA |
| | | TGTGGACAACACCCTAAACAATGTGATTATTTCTGTGA |
| | | AAATAAGCTTCGATCT |
| SEQ ID NO: 67 | K77E | AGTCTCTCTTGCAGGAAAGAGCAAGGCGAATTTTACG |
| | | ACCATCTTTTGCGAGACTGTATAAGCTGTGCGTCTATT |
| | | TGTGGACAACACCCTAAACAATGTGCTTATTTCTGTGA |
| | | AAATAAGCTTCGATCT |
| SEQ ID NO: 68 | R84Q, F103V | AGTCTCTCTTGCAGGAAAGAGCAAGGCAAATTTTACG |
| | | ACCATCTTTTGCAAGACTGTATAAGCTGTGCGTCTATT |
| | | TGTGGACAACACCCTAAACAATGTGCTTATGTCTGTGA |
| | | AAATAAGCTTCGATCT |
| SEQ ID NO: 69 | K77E, Q95R, | AGTCTCTCTTGCAGGAAAGAGCAAGGCGAATTTTACG |
| | A101D | ACCATCTTTTGCGAGACTGTATAAGCTGTGCGTCTATT |
| | | TGTGGACGACACCCTAAACAATGTGATTATTTCTGTGA |
| | | AAATAAGCTTCGATCT |
| SEQ ID NO: 70 | I87M, A101D | AGTCTCTCTTGCAGGAAAGAGCAAGGCAAATTTTACG |
| | | ACCATCTTTTGCGAGACTGTATGAGCTGTGCGTCAATT |
| | | TGTGGACAACACCCTAAACAATGTGATTATTTCTGTGA |
| | | AAATAAGCTTCGATCT |
| SEQ ID NO: 71 | IgG1 Fc | epkscdkthtcppcpapellggpsvflfppkpkdtlmisrtpe |
| | | vtcvvvdvshedpevkfnwyvdgvevhnaktkpreeqynstyr |
| | | vvsvltvlhqdwlngkeykckvsnkalpapiektiskakgqpr |
| | | epqvytlppsreemtknqvsltclvkgfypsdiavewesngqp |
| | | ennykttppvldsdgsfflyskltvdksrwqqgnvfscsvmhe |
| | | alhnhytqkslslspgk |
| SEQ ID NO: 72 | FLAG-tag | DYKDDDDK |
| SEQ ID NO: 73 | Fc | EPKSSDKTHTCPPCPAPEAEGAPSVFLFPPKPKDTLMISRT |
| | C220S/L234A/L235E/ | PEVTCVVVDVSHEDPEVKFNWYVDGVEVHNAKTKPREE |
| | G237A/K447del | QYNSTYRVVSVLTVLHQDWLNGKEYKCKVSNKALPAPI |
| | | EKTISKAKGQPREPQVYTLPPSRDELTKNQVSLTCLVKGF |
| | | YPSDIAVEWESNGQPENNYKTTPPVLDSDGSFFLYSKLTV |
| | | DKSRWQQGNVFSCSVMHEALHNHYTQKSLSLSPG |
| SEQ ID NO: 74 | Linker | GSGGGGS |
| SEQ ID NO: 75 | Fc | EPKSSDKTHTCPPCPAPEAEGAPSVFLFPPKPKDTLMISRT |
| | C220S/L234A/L235E/ | PEVTC |
| | G237A/E356D/M358L | VVVDVSHEDPEVKFNWYVDGVEVHNAKTKPREEQYNS |
| | | TYRVVSVLTVLHQDWLNGKEYKC |
| | | KVSNKALPAPIEKTISKAKGQPREPQVYTLPPSRDELTKN |
| | | QVSLTCLVKGFYPSDIAVEW |
| | | ESNGQPENNYKTTPPVLDSDGSFFLYSKLTVDKSRWQQG |
| | | NVFSCSVMHEALHNHYTQKSL |
| | | SLSPGK |
| SEQ ID NO: 76 | Linker | GSGGS |
| SEQ ID NO: 77 | Linker | GGGGS |
| SEQ ID NO: 78 | Linker | GGGGSGGGGS |
| SEQ ID NO: 79 | Linker | GGGGSGGGGSGGGGS |
| SEQ ID NO: 80 | Linker | GGGGSSA |
| SEQ ID NO: 81 | Fc (E356D and | EPKSSDKTHTCPPCPAPELLGGPSVFLFPPKPKDTLMISRT |
| | M358L) | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVYTLPPSRDELTKNQVSLTCLVKGF |
| | | YPSDIAVEWESNGQPENNYKTTPPVLDSDGSFFLYSKLTV |
| | | DKSRWQQGNVFSCSVMHEALHNHYTQKSLSLSPG |
| SEQ ID NO: 82 | Fc (C5S (C220S), | EPKSSDKTHTCPPCPAPELLGGPSVFLFPPKPKDTLMISRT |
| | R77C, (R292C), | PEVTCVVVDVSHEDPEVKFNWYVDGVEVHNAKTKPCEE |
| | N82G (N297G), | QYGSTYRCVSVLTVLHQDWLNGKEYKCKVSNKALPAPI |
| | V87C (V302C)) | EKTISKAKGQPREPQVYTLPPSREEMTKNQVSLTCLVKG |
| | | FYPSDIAVEWESNGQPENNYKTTPPVLDSDGSFFLYSKLT |
| | | VDKSRWQQGNVFSCSVMHEALHNHYTQKSLSLSPGK |
| SEQ ID NO: 83 | Fc with | EPKSSDKTHTCPPCPAPEAEGAPSVFLFPPKPKDTLMISRT |
| | C220S/L234A/L235E/ | PEVTC |
| | G237A | VVVDVSHEDPEVKFNWYVDGVEVHNAKTKPREEQYNS |
| | | TYRVVSVLTVLHQDWLNGKEYKC |
| | | KVSNKALPAPIEKTISKAKGQPREPQVYTLPPSREEMTKN |
| | | QVSLTCLVKGFYPSDIAVEW |
| | | ESNGQPENNYKTTPPVLDSDGSFFLYSKLTVDKSRWQQG |
| | | NVFSCSVMHEALHNHYTQKSL |
| | | SLSPGK |
| SEQ ID NO: 84 | Linker | GGGGSGGGGSGGGGSGGGGS |
| SEQ ID NO: 85 | Fc1 (G4S)2 (TACI | EPKSSDKTHTCPPCPAPEAEGAPSVFLFPPKPKDTLM |
| | WT (29-110)) | ISRTPEVTCVVVDVSHEDPEVKFNWYVDGVEVHNAKTK |
| | | PREEQYNSTYRVVSVLTVLHQD |
| | | WLNGKEYKCKVSNKALPAPIEKTISKAKGQPREPQVYTL |
| | | PPSRDELTKNQVSLTCLVKGF |
| | | YPSDIAVEWESNGQPENNYKTTPPVLDSDGSFFLYSKLTV |
| | | DKSRWQQGNVFSCSVMHEAL |
| | | HNHYTQKSLSLSPGGGGGSGGGGSVAMRSCPEEQYWDP |
| | | LLGTCMSCKTICNHQSQRTCAA |
| | | FCRSLSCRKEQGKFYDHLLRDCISCASICGQHPKQCAYFC |
| | | ENKLRS |
| SEQ ID NO: 86 | Fc1.1 (G4S)4 (TACI | EPKSSDKTHTCPPCPAPEAEGAPSVFLFPPKPKDTLMISRT |
| | H1788 (68-110)) | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVYTLPPSRDELTKNQVSLTCLVKGF |
| | | YPSDIAVEWESNGQPENNYKTTPPVLDSDGSFFLYSKLTV |
| | | DKSRWQQGNVFSCSVMHEALHNHYTQKSLSLSPGGGGG |
| | | SGGGGSGGGGSGGGGSSLSCRKEQGEYYDHLLRDCISCA |
| | | SICGQHPKQCADFCENKLRS |
| SEQ ID NO: 87 | Linker | EAAAKEAAAKEAAAKEAAAKEAAAKEAAAK |
| SEQ ID NO: 88 | full length TACI | MSGLGRSRRGGRSRVDQEERFPQGLWTGVAMRSCPEEQ |
| | | YWDPLLGTCMSCKTICNHQSQR |
| | | TCAAFCRSLSCRKEQGKFYDHLLRDCISCASICGQHPKQC |
| | | AYFCENKLRSPVNLPPELRR |
| | | QRSGEVENNSDNSGRYQGLEHRGSEASPALPGLKLSADQ |
| | | VALVYSTLGLCLCAVLCCFLV |
| | | AVACFLKKRGDPCSCQPRSRPRQSPAKSSQDHAMEAGSP |
| | | VSTSPEPVETCSFCFPECRAP |
| | | TQESAVTPGTPDPTCAGRWGCHTRTTVLQPCPHIPDSGL |
| | | GIVCVPAQEGGPGA |
| SEQ ID NO: 89 | knob Fc | EPKSSDKTHTCPPCPAPEAEGAPSVFLFPPKPKDTLMISRT |
| | | PEVTCVVVDVSHEDPEVKENWYVDGVEVHNAKTKPREE |
| | | QYNSTYRVVSVLTVLHQDWLNGKEYKCKVSNKALPAPI |
| | | EKTISKAKGQPREPQVYTLPPCREEMTKNQVSLWCLVKG |
| | | FYPSDIAVEWESNGQPENNYKTTPPVLDSDGSFFLYSKLT |
| | | VDKSRWQQGNVFSCSVMHEALHNHYTQKSLSLSPG |
| SEQ ID NO: 90 | hole Fc | EPKSSDKTHTCPPCPAPEAEGAPSVFLFPPKPKDTLMISRT |
| | | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVCTLPPSREEMTKNQVSLSCAVKG |
| | | FYPSDIAVEWESNGQPENNYKTTPPVLDSDGSFFL VSKLT |
| | | VDKSRWQQGNVFSCSVMHEALHNHYTQKSLSLSG |
| SEQ ID NO: 91 | Linker | GGGGSGGGGSGGGGSGGGGSGGGGS |
| SEQ ID NO: 92 | L82P | SLSCRKEQGKFYDHPLRDCISCASICGQHPKQCAYFCENK |
| | | LRS |
| SEQ ID NO: 93 | D85E, K98T | SLSCRKEQGKFYDHLLRECISCASICGQHPTQCAYFCENK |
| | | LRS |
| SEQ ID NO: 94 | I87L, K98T | SLSCRKEQGKFYDHLLRDCLSCASICGQHPTQCAYFCEN |
| | | KLRS |
| SEQ ID NO: 95 | A101D | SLSCRKEQGKFYDHLLRDCISCASICGQHPKQCDYFCEN |
| | | KLRS |
| SEQ ID NO: 96 | C86Y | SLSCRKEQGKFYDHLLRDYISCASICGQHPKQCAYFCEN |
| | | KLRS |
| SEQ ID NO: 97 | L82P, I87L | SLSCRKEQGKFYDHPLRDCLSCASICGQHPKQCAYFCEN |
| | | KLRS |
| SEQ ID NO: 98 | G76S, P97S | SLSCRKEQSKFYDHLLRDCISCASICGQHSKQCAYFCENK |
| | | LRS |
| SEQ ID NO: 99 | L83S, F103S | SLSCRKEQGKFYDHLSRDCISCASICGQHPKQCAYSCENK |
| | | LRS |
| SEQ ID NO: 100 | L82H | SLSCRKEQGKFYDHHLRDCISCASICGQHPKQCAYFCEN |
| | | KLRS |
| SEQ ID NO: 101 | D85V | VAMRSCPEEQYWDPLLGTCMSCKTICNHQSQRTCAAFC |
| | | RSLSCRKEQGKFYDHLLRVCISCASICGQHPKQCAYFCEN |
| | | KLRS |
| SEQ ID NO: 102 | E74V | VAMRSCPEEQYWDPLLGTCMSCKTICNHQSQRTCAAFC |
| | | RSLSCRKVQGKFYDHLLRDCISCASICGQHPKQCAYFCE |
| | | NKLRS |
| SEQ ID NO: 103 | R84L | VAMRSCPEEQYWDPLLGTCMSCKTICNHQSQRTCAAFC |
| | | RSLSCRKEQGKFYDHLLLDCISCASICGQHPKQCAYFCEN |
| | | KLRS |
| SEQ ID NO: 104 | K77E, R84L, | VAMRSCPEEQYWDPLLGTCMSCKTICNHQSQRTCAAFC |
| | F103Y | RSLSCRKEQGEFYDHLLLDCISCASICGQHPKQCAYYCEN |
| | | KLRS |
| SEQ ID NO: 105 | Y79F, Q99E | VAMRSCPEEQYWDPLLGTCMSCKTICNHQSQRTCAAFC |
| | | RSLSCRKEQGKFFDHLLRDCISCASICGQHPKECAYFCEN |
| | | KLRS |
| SEQ ID NO: 106 | Y79F | VAMRSCPEEQYWDPLLGTCMSCKTICNHQSQRTCAAFC |
| | | RSLSCRKEQGKFFDHLLRDCISCASICGQHPKQCAYFCEN |
| | | KLRS |
| SEQ ID NO: 107 | R84G | VAMRSCPEEQYWDPLLGTCMSCKTICNHQSQRTCAAFC |
| | | RSLSCRKEQGKFYDHLLGDCISCASICGQHPKQCAYFCE |
| | | NKLRS |
| SEQ ID NO: 108 | A101D | VAMRSCPEEQYWDPLLGTCMSCKTICNHQSQRTCAAFC |
| | | RSLSCRKEQGKFYDHLLRDCISCASICGQHPKQCDYFCEN |
| | | KLRS |
| SEQ ID NO: 109 | K77E, R84Q | VAMRSCPEEQYWDPLLGTCMSCKTICNHQSQRTCAAFC |
| | | RSLSCRKEQGEFYDHLLQDCISCASICGQHPKQCAYFCEN |
| | | KLRS |
| SEQ ID NO: 110 | K77E, A101D | VAMRSCPEEQYWDPLLGTCMSCKTICNHQSQRTCAAFC |
| | | RSLSCRKEQGEFYDHLLRDCISCASICGQHPKQCDYFCEN |
| | | KLRS |
| SEQ ID NO: 111 | K77E, F78Y, | VAMRSCPEEQYWDPLLGTCMSCKTICNHQSQRTCAAFC |
| | Y102D | RSLSCRKEQGEYYDHLLRDCISCASICGQHPKQCADFCE |
| | | NKLRS |
| SEQ ID NO: 112 | Q75E, R84Q | VAMRSCPEEQYWDPLLGTCMSCKTICNHQSQRTCAAFC |
| | | RSLSCRKEEGKFYDHLLQDCISCASICGQHPKQCAYFCEN |
| | | KLRS |
| SEQ ID NO: 113 | Q75R, R84G, I92V | VAMRSCPEEQYWDPLLGTCMSCKTICNHQSQRTCAAFC |
| | | RSLSCRKERGKFYDHLLGDCISCASVCGQHPKQCAYFCE |
| | | NKLRS |
| SEQ ID NO: 114 | K77E, A101D, | VAMRSCPEEQYWDPLLGTCMSCKTICNHQSQRTCAAFC |
| | Y102D | RSLSCRKEQGEFYDHLLRDCISCASICGQHPKQCDDFCEN |
| | | KLRS |
| SEQ ID NO: 115 | R84Q | VAMRSCPEEQYWDPLLGTCMSCKTICNHQSQRTCAAFC |
| | | RSLSCRKEQGKFYDHLLQDCISCASICGQHPKQCAYFCE |
| | | NKLRS |
| SEQ ID NO: 116 | R84Q, S88N, | VAMRSCPEEQYWDPLLGTCMSCKTICNHQSQRTCAAFC |
| | A101D | RSLSCRKEQGKFYDHLLQDCINCASICGQHPKQCDYFCE |
| | | NKLRS |
| SEQ ID NO: 117 | K77E | VAMRSCPEEQYWDPLLGTCMSCKTICNHQSQRTCAAFC |
| | | RSLSCRKEQGEFYDHLLRDCISCASICGQHPKQCAYFCEN |
| | | KLRS |
| SEQ ID NO: 118 | R84Q, F103V | VAMRSCPEEQYWDPLLGTCMSCKTICNHQSQRTCAAFC |
| | | RSLSCRKEQGKFYDHLLQDCISCASICGQHPKQCAYVCE |
| | | NKLRS |
| SEQ ID NO: 119 | K77E, Q95R, | VAMRSCPEEQYWDPLLGTCMSCKTICNHQSQRTCAAFC |
| | A101D | RSLSCRKEQGEFYDHLLRDCISCASICGRHPKQCDYFCEN |
| | | KLRS |
| SEQ ID NO: 120 | I87M, A101D | VAMRSCPEEQYWDPLLGTCMSCKTICNHQSQRTCAAFC |
| | | RSLSCRKEQGKFYDHLLRDCMSCASICGQHPKQCDYFCE |
| | | NKLRS |
| SEQ ID NO: 121 | His tag | HHHHHH |
| SEQ ID NO: 122 | TACI ECD (1-166) | MSGLGRSRRGGRSRVDQEERFPQGLWTGVAMRSCPEEQ |
| | | YWDPLLGTCMSCKTICNHQSQRTCAAFCRSLSCRKEQG |
| | | KFYDHLLRDCISCASICGQHPKOCAYFCENKLRSPVNL |
| | | PPELRRQRSGEVENNSDNSGRYQGLEHRGSEASPALPGK |
| | | LSADQVALVYST |
| SEQ ID NO: 123 | 1xEAAAK | EAAAK |
| SEQ ID NO: 124 | 2xEAAAK | EAAAKEAAAK |
| SEQ ID NO: 125 | 3xEAAAK | EAAAKEAAAKEAAAK |
| SEQ ID NO: 126 | 4xEAAAK | EAAAKEAAAKEAAAKEAAAK |
| SEQ ID NO: 127 | 5xEAAAK | EAAAKEAAAKEAAAKEAAAKEAAAK |
| SEQ ID NO: 128 | knob Fc | EPKSSDKTHTCPPCPAPEAEGAPSVFLFPPKPKDTLMISRT |
| | | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVYTLPPCREEMTKNQVSLWCLVKG |
| | | FYPSDIAVEWESNGQPENNYKTTPPVLDSDGSFFLYSKLT |
| | | VDKSRWQQGNVFSCSVMHEALHNHYTQKSLSLSPGK |
| SEQ ID NO: 129 | hole Fc | EPKSSDKTHTCPPCPAPEAEGAPSVFLFPPKPKDTLMISRT |
| | | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVCTLPPSREEMTKNQVSLSCAVKG |
| | | FYPSDIAVEWESNGQPENNYKTTPPVLDSDGSFFLVSKLT |
| | | VDKSRWQQGNVFSCSVMHEALHNHYTQKSLSLSPGK |
| SEQ ID NO: 130 | atacicept (TACI | AMRSCPEEQYWDPLLGTCMSCKTICNHQSQRTCAAFCRS |
| | portion, 30-110) | LSCRKEQGKFYDHLLRDCISC |
| | | ASICGQHPKQCAYFCENKLRS |
| SEQ ID NO: 131 | telitacicept (TACI | SRVDQEER FPQGLWTGVA MRSCPEEQYW |
| | portion, 13-118) | DPLLGTCMSC |
| | | KTICNHQSQR TCAAFCRSLS CRKEQGKFYD |
| | | HLLRDCISCA SICGQHPKQC |
| | | AYFCENKLRS PVNLPPEL |
| SEQ ID NO: 132 | atacicept | AMRSCPEEQYWDPLLGTCMSCKTICNHQSQRTCAAFCRS |
| | | LSCRKEQGKFYDHLLRDCISC |
| | | ASICGQHPKQCAYFCENKLRSEPKSSDKTHTCPPCPAPEA |
| | | EGAPSVFLFPPKPKDTLMIS |
| | | RTPEVTCVVVDVSHEDPEVKFNWYVDGVEVHNAKTKPR |
| | | EEQYNSTYRVVSVLTVLHQDWL |
| | | NGKEYKCKVSNKALPSSIEKTISKAKGQPREPQVYTLPPS |
| | | RDELTKNQVSLTCLVKGFYP |
| | | SDIAVEWESNGQPENNYKTTPPVLDSDGSFFLYSKLTVD |
| | | KSRWQQGNVFSCSVMHEALHN |
| | | HYTQKSLSLSPGK |
| SEQ ID NO: 133 | linker | GGGGSGGGGSAAA |
| SEQ ID NO: 134 | Fc with | EPKSSDKTHTCPPCPAPPVAGPSVFLFPPKPKDTLMISRTP |
| | C220S/E233P/L234V/ | EVTC |
| | L235A/G236del/ | VVVDVKHEDPEVKFNWYVDGVEVHNAKTKPREEQYNS |
| | S267K | TYRVVSVLTVLHQDWLNGKEYKC |
| | | KVSNKALPAPIEKTISKAKGQPREPQVYTLPPSREEMTKN |
| | | QVSLTCLVKGFYPSDIAVEW |
| | | ESNGQPENNYKTTPPVLDSDGSFFLYSKLTVDKSRWQQG |
| | | NVFSCSVMHEALHNHYTQKSL |
| | | SLSPGK |
| SEQ ID NO: 135 | Fc (C5S (C220S), | EPKSSDKTHTCPPCPAPELLGGPSVFLFPPKPKDTLMISRT |
| | R77C, (R292C), | PEVTCVVVDVSHEDPEVKFNWYVDGVEVHNAKTKPCEE |
| | N82G (N297G), | QYGSTYRCVSVLTVLHQDWLNGKEYKCKVSNKALPAPI |
| | V87C (V302C), | EKTISKAKGQPREPQVYTLPPSREEMTKNQVSLTCLVKG |
| | L232del (K447del)) | FYPSDIAVEWESNGQPENNYKTTPPVLDSDGSFFLYSKLT |
| | | VDKSRWQQGNVFSCSVMHEALHNHYTQKSLSLSPG |
| SEQ ID NO: 136 | Fc with | EPKSSDKTHTCPPCPAPEAEGAPSVFLFPPKPKDTLMISRT |
| | C220S/L234A/L235E/ | PEVTC |
| | G237A/K447del | VVVDVSHEDPEVKFNWYVDGVEVHNAKTKPREEQYNS |
| | | TYRVVSVLTVLHQDWLNGKEYKC |
| | | KVSNKALPAPIEKTISKAKGQPREPQVYTLPPSREEMTKN |
| | | QVSLTCLVKGFYPSDIAVEW |
| | | ESNGQPENNYKTTPPVLDSDGSFFLYSKLTVDKSRWQQG |
| | | NVFSCSVMHEALHNHYTQKSL |
| | | SLSPG |
| SEQ ID NO: 137 | Fc with | EPKSSDKTHTCPPCPAPPVAGPSVFLFPPKPKDTLMISRTP |
| | C220S/E233P/L234V/ | EVTC |
| | L235A/G236del/ | VVVDVKHEDPEVKFNWYVDGVEVHNAKTKPREEQYNS |
| | S267K/K447del | TYRVVSVLTVLHQDWLNGKEYKC |
| | | KVSNKALPAPIEKTISKAKGQPREPQVYTLPPSREEMTKN |
| | | QVSLTCLVKGFYPSDIAVEW |
| | | ESNGQPENNYKTTPPVLDSDGSFFLYSKLTVDKSRWQQG |
| | | NVFSCSVMHEALHNHYTQKSL |
| | | SLSPG |
| SEQ ID NO: 138 | IgG2 Fc | TKVDKTVERKCCVECPPCPAPPVAGPSVFLFPPKPKDTL |
| | | MISRTPEVTCVVVDVSHEDPEVQFNWYVDGVEVHNAKT |
| | | KPREEQFNSTFRVVSVLTVVHQDWLNGKEYKCKVSNKG |
| | | LPAPIEKTISKTKGQPREPQVYTLPPSREEMTKNQVSLTCL |
| | | VKGFYPSDIAVEWESNGQPENNYKTTPPMLDSDGSFFLY |
| | | SKLTVDKSRWQQGNVFSCSVMHEALHNHYTQKSLSLSP |
| | | GK |
| SEQ ID NO: 139 | IgG4 Fc | ESKYGPPCPSCPAPEFLGGPSVFLFPPKPKDTLMISRTPEV |
| | | TCVVVDVSQED |
| | | PEVQFNWYVDGVEVHNAKTKPREEQFNSTYRVVSVLTV |
| | | LHQDWLNGKEY |
| | | KCKVSNKGLPSSIEKTISKAKGQPREPQVYTLPPSQEEMT |
| | | KNQVSLTCLVK |
| | | GFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 140 | IgG4 Fc S228P | ESKYGPPCPPCPAPEFLGGPSVFLFPPKPKDTLMISRTPEV |
| | | TCVVVDVSQED |
| | | PEVQFNWYVDGVEVHNAKTKPREEQFNSTYRVVSVLTV |
| | | LHQDWLNGKEY |
| | | KCKVSNKGLPSSIEKTISKAKGQPREPQVYTLPPSQEEMT |
| | | KNQVSLTCLVK |
| | | GFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 141 | Fc K chain | EPKSSDKTHTCPPCPAPEAEGAPSVFLFPPKPKDTLMISRT |
| | | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVYTLPPSRKKMTKNQVSLTCLVKG |
| | | FYPSDIAVEWESNGQPENNYKTTPPVLKSDGSFFLYSKLT |
| | | VDKSRWQQGNVFSCSVMHEALHNHYTQKSLSLSPG |
| SEQ ID NO: 142 | Fc D chain | EPKSSDKTHTCPPCPAPEAEGAPSVFLFPPKPKDTLMISRT |
| | | PEVTCVVVDVSH |
| | | EDPEVKFNWYVDGVEVHNAKTKPREEQYNSTYRVVSVL |
| | | TVLHQDWLNGKEYKCKVSNKALPAPIEKTISKAKGQPRE |
| | | PQVYTLPPSREEMTKNQVSLTCLVDGFYPSDIAVEWESN |
| | | GQPENNYDTTPPVLDSDGSFFLYSDLTVDKSRWQQGNVF |
| | | SCSVMHEALHNHYTQKSLSLSPG |
| SEQ ID NO: 143 | Green Fluorescent | SKGEELFTGVVPILVELDGDVNGHKFSVSGEGEGDATYG |
| | protein | KLTLKFICTTGKLPVPWPTL |
| | | VTTFSYGVQCFSRYPDHMKQHDFFKSAMPEGYVQERTIF |
| | | FKDDGNYKTRAEVKFEGDTLV |
| | | NRIELKGIDFKEDGNILGHKLEYNYNSHNVYIMADKQKN |
| | | GIKVNFKIRHNIEDGSVQLAD |
| | | HYQQNTPIGDGPVLLPDNHYLSTQSALSKDPNEKRDHM |
| | | VLLEFVTAAGITHGMDELYK |
| SEQ ID NO: 144 | eGFP | SKGEELFTGVVPILVELDGDVNGHKFSVSGEGEGDATYG |
| | | KLTLKFICTTGKLPVPWPTL |
| | | VTTLTYGVQCFSRYPDHMKQHDFFKSAMPEGYVQERTIF |
| | | FKDDGNYKTRAEVKFEGDTLV |
| | | NRIELKGIDFKEDGNILGHKLEYNYNSHNVYIMADKQKN |
| | | GIKVNFKIRHNIEDGSVQLAD |
| | | HYQQNTPIGDGPVLLPDNHYLSTQSALSKDPNEKRDHM |
| | | VLLEFVTAAGITHGMDELYK |
| SEQ ID NO: 145 | eGFP | MVSKGEELFTGVVPILVELDGDVNGHKFSVSGEGEGDAT |
| | | YGKLTLKFICTTGKLPVPWPTLVTTLTYGVQCFSRYPDH |
| | | MKQHDFFKSAMPEGYVQERTIFFKDDGNYKTRAEVKFE |
| | | GDTLVNRIELKGIDFKEDGNILGHKLEYNYNSHNVYIMA |
| | | DKQKNGIKVNFKIRHNIEDGSVQLADHYQQNTPIGDGPV |
| | | LLPDNHYLSTQSALSKDPNEKRDHMVLLEFVTAAGITLG |
| | | MDELYK |
| SEQ ID NO: 146 | Moiety: COMP | DLGPQMLRELQETNAALQDVRELLRQQVREITFLKNTV |
| | | MECDACG |
| SEQ ID NO: 147 | Moiety: VASP | DLQRVKQELLEEVKKELQKVKEEIIEAFVQELR |
| SEQ ID NO: 148 | Moiety: ZZ12.6 | DVQAISSTIQDLQDQVDSLAEVVLQNRRGLDLLTRSGGGS |
| SEQ ID NO: 149 | HSA signal peptide | MKWVTFISLLFLFSSAYS |
| SEQ ID NO: 150 | Ig kappa light chain | MDMRAPAGIFGFLLVLFPGYRS |
| SEQ ID NO: 151 | human azurocidin | MTRLTVLALLAGLLASSRA |
| | preprotein signal | |
| | sequence | |
| SEQ ID NO: 152 | IgG heavy chain | MELGLSWIFLLAILKGVQC |
| | signal peptide | |
| SEQ ID NO: 153 | IgG heavy chain | MELGLRWVFLVAILEGVQC |
| | signal peptide | |
| SEQ ID NO: 154 | IgG heavy chain | MKHLWFFLLLVAAPRWVLS |
| | signal peptide | |
| SEQ ID NO: 155 | IgG heavy chain | MDWTWRILFLVAAATGAHS |
| | signal peptide | |
| SEQ ID NO: 156 | IgG heavy chain | MDWTWRFLFVVAAATGVQS |
| | signal peptide | |
| SEQ ID NO: 157 | IgG heavy chain | MEFGLSWLFLVAILKGVQC |
| | signal peptide | |
| SEQ ID NO: 158 | IgG heavy chain | MEFGLSWVFLVALFRGVQC |
| | signal peptide | |
| SEQ ID NO: 159 | IgG heavy chain | MDLLHKNMKHLWFFLLLVAAPRWVLS |
| | signal peptide | |
| SEQ ID NO: 160 | IgG Kappa light | MDMRVPAQLLGLLLLWLSGARC |
| | chain signal | |
| | sequences | |
| SEQ ID NO: 161 | IgG Kappa light | MKYLLPTAAAGLLLLAAQPAMA |
| | chain signal | |
| | sequences | |
| SEQ ID NO: 162 | Gaussia luciferase | MGVKVLFALICIAVAEA |
| SEQ ID NO: 163 | Human albumin | MKWVTFISLLFLFSSAYS |
| SEQ ID NO: 164 | Human | MAFLWLLSCWALLGTTFG |
| | chymotrypsinogen | |
| SEQ ID NO: 165 | Human interleukin-2 | MQLLSCIALILALV |
| SEQ ID NO: 166 | Human trypsinogen-2 | MNLLLILTFVAAAVA |
| SEQ ID NO: 167 | TACI(541)-Fc_589 | SLSCRKEQGEYYDHLLRDCISCASICGQHPKQCADFC |
| | K77E, F78Y, | ENKLRSGSGGGGSEPKSSDKTHTCPPCPAPEAE |
| | Y102D | GAPSVFLFPPKPKDTLMISRTPEVTCVVVDVSHEDPEVKF |
| | | NWYVDGVEVHNAKTKPREEQYNSTYRVVSV |
| | | LTVLHQDWLNGKEYKCKVSNKALPAPIEKTISKAKGQPR |
| | | EPQVYTLPPSRDELTKNQVSLTCL VKGFYPS |
| | | DIAVEWESNGQPENNYKTTPPVLDSDGSFFLYSKLTVDK |
| | | SRWQQGNVFSCSVMHEALHNHYTQKSLSLSPG |
| SEQ ID NO: 168 | TACI(541)-Fc_597 | SLSCRKEQGEYYDHLLRDCISCASICGQHPKQCADFC |
| | K77E, F78Y, | ENKLRSGSGGGGSEPKSSDKTHTCPPCPAPELL |
| | Y102D | GGPSVFLFPPKPKDTLMISRTPEVTCVVVDVSHEDPEVKF |
| | | NWYVDGVEVHNAKTKPREEQYNSTYRVVSV |
| | | LTVLHQDWLNGKEYKCKVSNKALPAPIEKTISKAKGQPR |
| | | EPQVYTLPPSRDELTKNQVSLTCLVKGFYPS |
| | | DIAVEWESNGQPENNYKTTPPVLDSDGSFFLYSKLTVDK |
| | | SRWQQGNVFSCSVMHEALHNHYTQKSLSLSPG |
| SEQ ID NO: 169 | TACI(542)-Fc_589 | SLSCRKEEGKFYDHLLQDCISCASICGQHPKQCAYFCENK |
| | Q75E, R84Q | LRSGSGGGGSEPKSSDKTHTCPPCPAPEAE |
| | | GAPSVFLFPPKPKDTLMISRTPEVTCVVVDVSHEDPEVKF |
| | | NWYVDGVEVHNAKTKPREEQYNSTYRVVSV |
| | | LTVLHQDWLNGKEYKCKVSNKALPAPIEKTISKAKGQPR |
| | | EPQVYTLPPSRDELTKNQVSLTCL VKGFYPS |
| | | DIAVEWESNGQPENNYKTTPPVLDSDGSFFLYSKLTVDK |
| | | SRWQQGNVFSCSVMHEALHNHYTQKSLSLSPG |
| SEQ ID NO: 170 | TACI(542)-Fc_597 | SLSCRKEEGKFYDHLLQDCISCASICGQHPKQCAYFCENK |
| | Q75E, R84Q | LRSGSGGGGSEPKSSDKTHTCPPCPAPELL |
| | | GGPSVFLFPPKPKDTLMISRTPEVTCVVVDVSHEDPEVKF |
| | | NWYVDGVEVHNAKTKPREEQYNSTYRVVSV |
| | | LTVLHQDWLNGKEYKCKVSNKALPAPIEKTISKAKGQPR |
| | | EPQVYTLPPSRDELTKNQVSLTCL VKGFYPS |
| | | DIAVEWESNGQPENNYKTTPPVLDSDGSFFLYSKLTVDK |
| | | SRWQQGNVFSCSVMHEALHNHYTQKSLSLSPG |
| SEQ ID NO: 171 | TACI(528)-Fc_589 | SLSCRKEQGKFYDHLLRDCISCASICGQHPKQCAYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 172 | TACI(528)-Fc_597 | SLSCRKEQGKFYDHLLRDCISCASICGQHPKQCAYFCEN |
| | | KLRSGSGGGGSEPKSSDKTHTCPPCPAPELLGGPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALPAPIEKTISKAKGQPREPQVYTLPPSRDELTK |
| | | NQVSLTCLVKGFYPSDIAVEWESNGQPENNYKTTPPVLD |
| | | SDGSFFLYSKLTVDKSRWQQGNVFSCSVMHEALHNHYT |
| | | QKSLSLSPG |
| SEQ ID NO: 173 | Fc | DKPHT CPLCPAPELL GGPSVFLFPP KPKDTLMISR |
| | | TPEVTCVVVD |
| | | VSHEDPEVKF NWYVDGVEVH NAKTKPREEQ |
| | | YNSTYRVVSV LTVLHQDWLN |
| | | GKEYKCKVSN KALPAPIEKT ISKAKGQPRE |
| | | PQVYTLPPSR DELTKNQVSL |
| | | TCLVKGFYPS DIAVEWESNG QPENNYKATP |
| | | PVLDSDGSFF LYSKLTVDKS |
| | | RWQQGNVFSC SVMHEALHNH YTQKSLSLSP GK |
| SEQ ID NO: 174 | Fc | DPKSCDKPHT CPLCPAPELL GGPSVFLFPP |
| | | KPKDTLMISR TPEVTCVVVD |
| | | VSHEDPEVKF NWYVDGVEVH NAKTKPREEQ |
| | | YNSTYRVVSV LTVLHQDWLN |
| | | GKEYKCKVSN KALPAPIEKT ISKAKGQPRE |
| | | PQVYTLPPSR DELTKNQVSL |
| | | TCLVKGFYPS DIAVEWESNG QPENNYKATP |
| | | PVLDSDGSFF LYSKLTVDKS |
| | | RWQQGNVFSC SVMHEALHNH YTQKSLSLSP GK |
| SEQ ID NO: 175 | Fc variant | DKTH TCPPCPAPEA EGAPSVFLFP PKPKDTLMIS |
| | | RTPEVTCVVV |
| | | DVSHEDPEVK FNWYVDGVEV HNAKTKPREE |
| | | QYNSTYRVVS VLTVLHQDWL |
| | | NGKEYKCKVS NKALPSSIEK TISKAKGQPR |
| | | EPQVYTLPPS RDELTKNQVS |
| | | LTCLVKGFYP SDIAVEWESN GQPENNYKTT |
| | | PPVLDSDGSF FLYSKLTVDK |
| | | SRWQQGNVFS CSVMHEALHN HYTQKSLSLS PGK |
| SEQ ID NO: 176 | Fc variant | EPKSSDKTH TCPPCPAPEA EGAPSVFLFP PKPKDTLMIS |
| | | RTPEVTCVVV |
| | | DVSHEDPEVK FNWYVDGVEV HNAKTKPREE |
| | | QYNSTYRVVS VLTVLHQDWL |
| | | NGKEYKCKVS NKALPSSIEK TISKAKGQPR |
| | | EPQVYTLPPS RDELTKNQVS |
| | | LTCLVKGFYP SDIAVEWESN GQPENNYKTT |
| | | PPVLDSDGSF FLYSKLTVDK |
| | | SRWQQGNVFS CSVMHEALHN HYTQKSLSLS PGK |
| SEQ ID NO: 177 | N14132 | SLSCRKEEGKFYDHLLRDCISCASICGQHPKQCAYFCENK |
| | | LRS |
| SEQ ID NO: 178 | N14109 | SLSCRKEEGEFYDHLLRDCISCASICGQHPKQCAYFCENK |
| | | LRS |
| SEQ ID NO: 179 | N14110 | SLSCRKEEGKYYDHLLRDCISCASICGQHPKQCAYFCEN |
| | | KLRS |
| SEQ ID NO: 180 | N14111 | SLSCRKEEGKFYDHLLRDCISCASICGQHPKQCDYFCENK |
| | | LRS |
| SEQ ID NO: 181 | N14133 | SLSCRKEEGKFYDHLLRDCISCASICGQHPKQCADFCENK |
| | | LRS |
| SEQ ID NO: 182 | N14134 | SLSCRKEQGEYYDHLLQDCISCASICGQHPKQCAYFCEN |
| | | KLRS |
| SEQ ID NO: 183 | N14112 | SLSCRKEQGKYYDHLLRDCISCASICGQHPKQCAYFCEN |
| | | KLRS |
| SEQ ID NO: 184 | N14113 | SLSCRKEQGKYYDHLLQDCISCASICGQHPKQCAYFCEN |
| | | KLRS |
| SEQ ID NO: 185 | N14114 | SLSCRKEQGKYYDHLLRDCISCASICGQHPKQCDYFCEN |
| | | KLRS |
| SEQ ID NO: 186 | N14115 | SLSCRKEQGKYYDHLLRDCISCASICGQHPKQCADFCEN |
| | | KLRS |
| SEQ ID NO: 187 | N14130 | SLSCRKEQGKFYDHLLQDCISCASICGQHPKQCDYFCEN |
| | | KLRS |
| SEQ ID NO: 188 | N14131 | SLSCRKEQGKFYDHLLQDCISCASICGQHPKQCADFCEN |
| | | KLRS |
| SEQ ID NO: 189 | N14116 | SLSCRKEQGKFYDHLLRDCISCASICGQHPKQCDDFCEN |
| | | KLRS |
| SEQ ID NO: 190 | N13946 | SLSCRKEQGKFYDHLLRDCISCASICGQHPKQCADFCEN |
| | | KLRS |
| SEQ ID NO: 191 | N13944 | SLSCRKEQGEYYDHLLRDCISCASICGQHPKQCAYFCEN |
| | | KLRS |
| SEQ ID NO: 192 | N13945 | SLSCRKEQGEFYDHLLRDCISCASICGQHPKQCADFCENK |
| | | LRS |
| SEQ ID NO: 193 | IgG1 variant | DKPHTCPLCPAPELLGGPSVFLFPPKPKDTLMISRTPEVTC |
| | | VVVDVSHEDPEVKFNWYVDGVEVHNAKTKPREEQYNS |
| | | TYRVVSVLTVLHQDWLNGKEYKCKVSNKALPAPIEKTIS |
| | | KAKGQPREPQVYTLPPSRDELTKNQVSLTCLVKGFYPSDI |
| | | AVEWESNGQPENNYKATPPVLDSDGSFFLYSKLTVDKSR |
| | | WQQGNVFSCSVMHEALHNHYTQKSLSLSPGK |
| SEQ ID NO: 194 | linker | GSGGGGSGGGGS |
| SEQ ID NO: 195 | linker | (GGGGS)x13 |
| SEQ ID NO: 196 | TACI WT 13 | SLSCRKEQGKFYDHLLRDCISCASICGQHPKQCAYFCEN |
| | GS(G4S)2 (194) | KLRSGSGGGGSGGGGSEPKSSDKTHTCPPCPAPEAEGAPS |
| | sc_Fc 218 | VFLFPPKPKDTLMISRTPEVTCVVVDVSHEDPEVKFNWY |
| | | VDGVEVHNAKTKPREEQYNSTYRVVSVLTVLHQDWLN |
| | | GKEYKCKVSNKALPAPIEKTISKAKGQPREPQVYTLPPSR |
| | | DELTKNQVSLTCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSGGGGSGGGGSGGGGSGGGG |
| | | SGGGGSGGGGSGGGGSGGGGSGGGGSGGGGSGGGGSEP |
| | | KSSDKTHTCPPCPAPEAEGAPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALPAPIE |
| | | KTISKAKGQPREPQVYTLPPSRDELTKNQVSLTCLVKGFY |
| | | PSDIAVEWESNGQPENNYKTTPPVLDSDGSFFLYSKLTVD |
| | | KSRWQQGNVFSCSVMHEALHNHYTQKSLSLSPG |
| SEQ ID NO: 197 | TACI(528)-Fc_589 | SLSCRKEQGKFYDHLLRDCISCASICGQHPKQCAYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 198 | TACI WT 13 | SLSCRKEQGKFYDHLLRDCISCASICGQHPKQCAYFCEN |
| | (G4S)4 (84) (TACI | KLRSGGGGSGGGGSGGGGSGGGGSSLSCRKEQGKFYDH |
| | WT 13 GSG4S (74) | LLRDCISCASICGQHPKQCAYFCENKLRSGSGGGGSEPKS |
| | Fc 73 | SDKTHTCPPCPAPEAEGAPSVFLFPPKPKDTLMISRTPEVT |
| | | CVVVDVSHEDPEVKFNWYVDGVEVHNAKTKPREEQYN |
| | | STYRVVSVLTVLHQDWLNGKEYKCKVSNKALPAPIEKTI |
| | | SKAKGQPREPQVYTLPPSRDELTKNQVSLTCLVKGFYPS |
| | | DIAVEWESNGQPENNYKTTPPVLDSDGSFFLYSKLTVDK |
| | | SRWQQGNVFSCSVMHEALHNHYTQKSLSLSPG |
| SEQ ID NO: 199 | TACI 26 GS(G4S)2 | SLSCRKEQGEYYDHLLRDCISCASICGQHPKQCADFCEN |
| | (194) scFc_218 | KLRSGSGGGGSGGGGSEPKSSDKTHTCPPCPAPEAEGAPS |
| | | VFLFPPKPKDTLMISRTPEVTCVVVDVSHEDPEVKFNWY |
| | | VDGVEVHNAKTKPREEQYNSTYRVVSVLTVLHQDWLN |
| | | GKEYKCKVSNKALPAPIEKTISKAKGQPREPQVYTLPPSR |
| | | DELTKNQVSLTCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSGGGGSGGGGSGGGGSGGGG |
| | | SGGGGSGGGGSGGGGSGGGGSGGGGSGGGGSGGGGSEP |
| | | KSSDKTHTCPPCPAPEAEGAPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALPAPIE |
| | | KTISKAKGQPREPQVYTLPPSRDELTKNQVSLTCLVKGFY |
| | | PSDIAVEWESNGQPENNYKTTPPVLDSDGSFFLYSKLTVD |
| | | KSRWQQGNVFSCSVMHEALHNHYTQKSLSLSPG |
| SEQ ID NO: 200 | TACI(541)-Fc_589 | SLSCRKEQGEYYDHLLRDCISCASICGQHPKQCADFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 201 | TACI 26 GSG4S | SLSCRKEQGEYYDHLLRDCISCASICGQHPKQCADFCEN |
| | (74) Fc 73 (G4S)4 | KLRSGSGGGGSEPKSSDKTHTCPPCPAPEAEGAPSVFLFP |
| | (84) TACI 26 | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPGGGGGSGGGGSGGGGSGGGGSSLSCRKEQG |
| | | EYYDHLLRDCISCASICGQHPKQCADFCENKLRS |
| SEQ ID NO: 202 | TACI WT 13 | SLSCRKEQGKFYDHLLRDCISCASICGQHPKQCAYFCEN |
| | GSG4S (74) Fc | KLRSGSGGGGSEPKSSDKTHTCPPCPAPEAEGAPSVFLFP |
| | 73(G4S)4 (84) | PKPKDTLMISRTPEVTCVVVDVSHEDPEVKFNWYVDGV |
| | (TACI WT 13 | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPGGGGGSGGGGSGGGGSGGGGSSLSCRKEQG |
| | | KFYDHLLRDCISCASICGQHPKQCAYFCENKLRS |
| SEQ ID NO: 203 | TACI WT 1 | VAMRSCPEEQYWDPLLGTCMSCKTICNHQSQRTCAAFC |
| | GS(G4S)2 (194) | RSLSCRKEQGKFYDHLLRDCISCASICGQHPKQCAYFCEN |
| | scFc_218 | KLRSGSGGGGSGGGGSEPKSSDKTHTCPPCPAPEAEGAPS |
| | | VFLFPPKPKDTLMISRTPEVTCVVVDVSHEDPEVKFNWY |
| | | VDGVEVHNAKTKPREEQYNSTYRVVSVLTVLHQDWLN |
| | | GKEYKCKVSNKALPAPIEKTISKAKGQPREPQVYTLPPSR |
| | | DELTKNQVSLTCLVKGFYPSDIAVEWESNGQPENNYKTT |
| | | PPVLDSDGSFFLYSKLTVDKSRWQQGNVFSCSVMHEAL |
| | | HNHYTQKSLSLSPGGGGGSGGGGSGGGGSGGGGSGGGG |
| | | SGGGGSGGGGSGGGGSGGGGSGGGGSGGGGSGGGGSEP |
| | | KSSDKTHTCPPCPAPEAEGAPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALPAPIE |
| | | KTISKAKGQPREPQVYTLPPSRDELTKNQVSLTCLVKGFY |
| | | PSDIAVEWESNGQPENNYKTTPPVLDSDGSFFLYSKLTVD |
| | | KSRWQQGNVFSCSVMHEALHNHYTQKSLSLSPG |
| SEQ ID NO: 204 | | AMRSCPEEQYWDPLLGTCMSCKTICNHQSQRTCAAFCRS |
| | | LSCRKEQGKFYDHLLRDCISCASICGQHPKQCAYFCENK |
| | | LRSEPKSSDKTHTCPPCPAPEAEGAPSVFLFPPKPKDTLMI |
| | | SRTPEVTCVVVDVSHEDPEVKFNWYVDGVEVHNAKTKP |
| | | REEQYNSTYRVVSVLTVLHQDWLNGKEYKCKVSNKALP |
| | | SSIEKTISKAKGQPREPQVYTLPPSRDELTKNQVSLTCLV |
| | | KGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 205 | TACI WT 131 | SRVDQEERFPQGLWTGVAMRSCPEEQYWDPLLGTCMSC |
| | GS(G4S)2 (194) | KTICNHQSQRTCAAFCRSLSCRKEQGKFYDHLLRDCISCA |
| | scFc 218 | SICGQHPKQCAYFCENKLRSPVNLPPELGSGGGGSGGGG |
| | | SEPKSSDKTHTCPPCPAPEAEGAPSVFLFPPKPKDTLMISR |
| | | TPEVTCVVVDVSHEDPEVKFNWYVDGVEVHNAKTKPRE |
| | | EQYNSTYRVVSVLTVLHQDWLNGKEYKCKVSNKALPAP |
| | | IEKTISKAKGQPREPQVYTLPPSRDELTKNQVSLTCLVKG |
| | | FYPSDIAVEWESNGQPENNYKTTPPVLDSDGSFFLYSKLT |
| | | VDKSRWQQGNVFSCSVMHEALHNHYTQKSLSLSPGGGG |
| | | GSGGGGSGGGGSGGGGSGGGGSGGGGSGGGGSGGGGS |
| | | GGGGSGGGGSGGGGSGGGGSEPKSSDKTHTCPPCPAPEA |
| | | EGAPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPEN |
| | | NYKTTPPVLDSDGSFFLYSKLTVDKSRWQQGNVFSCSV |
| | | MHEALHNHYTQKSLSLSPG |
| SEQ ID NO: 206 | | SRVDQEERFPQGLWTGVAMRSCPEEQYWDPLLGTCMSC |
| | | KTICNHQSQRTCAAFCRSLSCRKEQGKFYDHLLRDCISCA |
| | | SICGQHPKQCAYFCENKLRSPVNLPPELDKTHTCPPCPAP |
| | | EAEGAPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPSSIEKTISKAKGQPREPQV |
| | | YTLPPSRDELTKNQVSLTCLVKGFYPSDIAVEWESNGQPE |
| | | NNYKTTPPVLDSDGSFFLYSKLTVDKSRWQQGNVFSCSV |
| | | MHEALHNHYTQKSLSLSPGK |
| SEQ ID NO: 207 | TACI WT 13 | AGTCTCTCTTGCAGGAAAGAGCAAGGCAAATTTTACG |
| | GS(G4S)2 (194) | ACCATCTTTTGCGAGACTGTATAAGCTGTGCGTCTATT |
| | sc_Fc 218 | TGTGGACAACACCCTAAACAATGTGCTTATTTCTGTGA |
| | | AAATAAGCTTCGATCTGGATCCGGTGGTGGCGGGTCA |
| | | GGGGGAGGTGGAAGCGAGCCCAAGTCCTCAGATAAGA |
| | | CTCATACTTGCCCCCCCTGTCCCGCCCCTGAGGCCGAA |
| | | GGCGCACCGTCAGTGTTTTTGTTCCCACCAAAGCCTAA |
| | | GGACACACTGATGATAAGCAGAACACCTGAGGTAACC |
| | | TGTGTTGTTGTGGATGTAAGCCACGAAGATCCGGAGG |
| | | TTAAGTTTAACTGGTACGTGGACGGTGTAGAAGTCCAT |
| | | AATGCTAAGACGAAGCCGAGGGAGGAACAATACAACT |
| | | CCACGTATAGAGTGGTCTCCGTCTTGACCGTACTCCAT |
| | | CAAGACTGGCTCAACGGGAAAGAGTACAAGTGTAAAG |
| | | TCTCTAACAAAGCTCTTCCTGCACCGATTGAGAAAACC |
| | | ATATCTAAAGCCAAGGGACAACCGAGAGAACCACAGG |
| | | TTTACACGCTCCCCCCCAGTAGAGACGAACTTACAAA |
| | | AAACCAGGTCAGTCTTACCTGCCTCGTCAAAGGCTTCT |
| | | ACCCCTCTGACATCGCTGTTGAGTGGGAATCTAACGGC |
| | | CAACCTGAGAACAATTACAAAACAACCCCGCCTGTCC |
| | | TTGACTCAGACGGTTCCTTTTTTCTTTACAGCAAGCTG |
| | | ACCGTCGACAAATCACGGTGGCAACAAGGCAATGTGT |
| | | TTTCCTGTTCCGTGATGCACGAGGCACTGCACAACCAC |
| | | TACACTCAAAAATCCCTTTCCCTTTCCCCAGGGGGAGG |
| | | TGGAGGGAGCGGTGGAGGTGGTAGCGGGGGTGGAGG |
| | | CTCAGGTGGTGGGGGTTCCGGCGGTGGCGGAAGTGGA |
| | | GGCGGTGGCTCTGGTGGTGGCGGATCTGGCGGAGGAG |
| | | GCAGCGGCGGAGGTGGGTCTGGGGGTGGAGGCTCCGG |
| | | AGGCGGGGGAAGCGGTGGAGGAGGGTCAGAGCCCAA |
| | | AAGCTCCGACAAGACTCACACATGCCCCCCTTGTCCAG |
| | | CGCCTGAAGCTGAGGGTGCGCCCTCTGTCTTCCTTTTC |
| | | CCCCCTAAGCCGAAAGATACCCTGATGATCTCCCGCAC |
| | | TCCCGAAGTCACATGTGTTGTTGTCGACGTATCTCATG |
| | | AAGATCCTGAGGTGAAATTCAACTGGTATGTAGACGG |
| | | GGTCGAAGTTCATAATGCTAAGACTAAGCCACGAGAA |
| | | GAGCAATACAACTCAACGTATCGGGTGGTGAGCGTTC |
| | | TGACGGTTCTGCACCAAGATTGGCTTAATGGAAAAGA |
| | | GTATAAGTGCAAGGTGTCCAACAAGGCTCTTCCGGCA |
| | | CCCATCGAAAAGACGATTTCCAAAGCGAAAGGCCAAC |
| | | CTAGGGAACCGCAAGTTTACACTTTGCCCCCGTCAAGA |
| | | GACGAACTTACCAAGAATCAAGTTTCCCTGACGTGCCT |
| | | TGTGAAGGGCTTCTACCCTAGCGATATAGCAGTTGAGT |
| | | GGGAATCTAACGGCCAGCCCGAAAATAATTATAAGAC |
| | | TACTCCGCCCGTGCTGGACAGTGATGGTTCATTTTTCC |
| | | TGTATTCAAAACTCACTGTGGACAAATCTAGATGGCA |
| | | GCAGGGTAATGTGTTCTCTTGTTCAGTTATGCACGAGG |
| | | CATTGCACAATCACTATACGCAAAAAAGTTTGTCTCTC |
| | | TCTCCGGGG |
| SEQ ID NO: 208 | TACI(541)-Fc_589 | AGTCTCTCTTGCAGGAAAGAGCAAGGCAAATTTTACG |
| | | ACCATCTTTTGCGAGACTGTATAAGCTGTGCGTCTATT |
| | | TGTGGACAACACCCTAAACAATGTGCTTATTTCTGTGA |
| | | AAATAAGCTTCGATCTGGATCCGGTGGAGGAGGGTCA |
| | | GAGCCCAAAAGCTCCGACAAGACTCACACATGCCCCC |
| | | CTTGTCCAGCGCCTGAAGCTGAGGGTGCGCCCTCTGTC |
| | | TTCCTTTTCCCCCCTAAGCCGAAAGATACCCTGATGAT |
| | | CTCCCGCACTCCCGAAGTCACATGTGTTGTTGTCGACG |
| | | TATCTCATGAAGATCCTGAGGTGAAATTCAACTGGTAT |
| | | GTAGACGGGGTCGAAGTTCATAATGCTAAGACTAAGC |
| | | CACGAGAAGAGCAATACAACTCAACGTATCGGGTGGT |
| | | GAGCGTTCTGACGGTTCTGCACCAAGATTGGCTTAATG |
| | | GAAAAGAGTATAAGTGCAAGGTGTCCAACAAGGCTCT |
| | | TCCGGCACCCATCGAAAAGACGATTTCCAAAGCGAAA |
| | | GGCCAACCTAGGGAACCGCAAGTTTACACTTTGCCCCC |
| | | GTCAAGAGACGAACTTACCAAGAATCAAGTTTCCCTG |
| | | ACGTGCCTTGTGAAGGGCTTCTACCCTAGCGATATAGC |
| | | AGTTGAGTGGGAATCTAACGGCCAGCCCGAAAATAAT |
| | | TATAAGACTACTCCGCCCGTGCTGGACAGTGATGGTTC |
| | | ATTTTTCCTGTATTCAAAACTCACTGTGGACAAATCTA |
| | | GATGGCAGCAGGGTAATGTGTTCTCTTGTTCAGTTATG |
| | | CACGAGGCATTGCACAATCACTATACGCAAAAAAGTT |
| | | TGTCTCTCTCTCCGGGG |
| SEQ ID NO: 209 | TACI WT 13 | AGTCTCTCTTGCAGGAAAGAGCAAGGCAAATTTTACG |
| | (G4S)4 (84) (TACI | ACCATCTTTTGCGAGACTGTATAAGCTGTGCGTCTATT |
| | WT 13 GSG4S (74) | TGTGGACAACACCCTAAACAATGTGCTTATTTCTGTGA |
| | Fc 73 | AAATAAGCTTCGATCTGGGGGTGGCGGAtcaGGAGGTG |
| | | GCGGTtcaGGCGGAGGAGGttctGGGGGTGGGGGTTCTTC |
| | | ATTGTCCTGCAGAAAGGAACAGGGGAAATTTTACGAT |
| | | CACTTGCTTAGAGATTGTATAAGCTGCGCGAGCATTTG |
| | | CGGGCAACACCCTAAACAGTGTGCGTATTTCTGCGAG |
| | | AATAAACTCCGGTCTGGATCCGGTGGAGGAGGGTCAG |
| | | AGCCCAAAAGCTCCGACAAGACTCACACATGCCCCCC |
| | | TTGTCCAGCGCCTGAAGCTGAGGGTGCGCCCTCTGTCT |
| | | TCCTTTTCCCCCCTAAGCCGAAAGATACCCTGATGATC |
| | | TCCCGCACTCCCGAAGTCACATGTGTTGTTGTCGACGT |
| | | ATCTCATGAAGATCCTGAGGTGAAATTCAACTGGTATG |
| | | TAGACGGGGTCGAAGTTCATAATGCTAAGACTAAGCC |
| | | ACGAGAAGAGCAATACAACTCAACGTATCGGGTGGTG |
| | | AGCGTTCTGACGGTTCTGCACCAAGATTGGCTTAATGG |
| | | AAAAGAGTATAAGTGCAAGGTGTCCAACAAGGCTCTT |
| | | CCGGCACCCATCGAAAAGACGATTTCCAAAGCGAAAG |
| | | GCCAACCTAGGGAACCGCAAGTTTACACTTTGCCCCCG |
| | | TCAAGAGACGAACTTACCAAGAATCAAGTTTCCCTGA |
| | | CGTGCCTTGTGAAGGGCTTCTACCCTAGCGATATAGCA |
| | | GTTGAGTGGGAATCTAACGGCCAGCCCGAAAATAATT |
| | | ATAAGACTACTCCGCCCGTGCTGGACAGTGATGGTTCA |
| | | TTTTTCCTGTATTCAAAACTCACTGTGGACAAATCTAG |
| | | ATGGCAGCAGGGTAATGTGTTCTCTTGTTCAGTTATGC |
| | | ACGAGGCATTGCACAATCACTATACGCAAAAAAGTTT |
| | | GTCTCTCTCTCCGGGG |
| SEQ ID NO: 210 | TACI 26 GS(G4S)2 | AGTCTCTCTTGCAGGAAAGAGCAAGGCGAATATTACG |
| | (194) scFc_218 | ACCATCTTTTGCGAGACTGTATAAGCTGTGCGTCTATT |
| | | TGTGGACAACACCCTAAACAATGTGCTGATTTCTGTGA |
| | | AAATAAGCTTCGATCTGGATCCGGTGGTGGCGGGTCA |
| | | GGGGGAGGTGGAAGCGAGCCCAAGTCCTCAGATAAGA |
| | | CTCATACTTGCCCCCCCTGTCCCGCCCCTGAGGCCGAA |
| | | GGCGCACCGTCAGTGTTTTTGTTCCCACCAAAGCCTAA |
| | | GGACACACTGATGATAAGCAGAACACCTGAGGTAACC |
| | | TGTGTTGTTGTGGATGTAAGCCACGAAGATCCGGAGG |
| | | TTAAGTTTAACTGGTACGTGGACGGTGTAGAAGTCCAT |
| | | AATGCTAAGACGAAGCCGAGGGAGGAACAATACAACT |
| | | CCACGTATAGAGTGGTCTCCGTCTTGACCGTACTCCAT |
| | | CAAGACTGGCTCAACGGGAAAGAGTACAAGTGTAAAG |
| | | TCTCTAACAAAGCTCTTCCTGCACCGATTGAGAAAACC |
| | | ATATCTAAAGCCAAGGGACAACCGAGAGAACCACAGG |
| | | TTTACACGCTCCCCCCCAGTAGAGACGAACTTACAAA |
| | | AAACCAGGTCAGTCTTACCTGCCTCGTCAAAGGCTTCT |
| | | ACCCCTCTGACATCGCTGTTGAGTGGGAATCTAACGGC |
| | | CAACCTGAGAACAATTACAAAACAACCCCGCCTGTCC |
| | | TTGACTCAGACGGTTCCTTTTTTCTTTACAGCAAGCTG |
| | | ACCGTCGACAAATCACGGTGGCAACAAGGCAATGTGT |
| | | TTTCCTGTTCCGTGATGCACGAGGCACTGCACAACCAC |
| | | TACACTCAAAAATCCCTTTCCCTTTCCCCAGGGGGAGG |
| | | TGGAGGGAGCGGTGGAGGTGGTAGCGGGGGTGGAGG |
| | | CTCAGGTGGTGGGGGTTCCGGCGGTGGCGGAAGTGGA |
| | | GGCGGTGGCTCTGGTGGTGGCGGATCTGGCGGAGGAG |
| | | GCAGCGGCGGAGGTGGGTCTGGGGGTGGAGGCTCCGG |
| | | AGGCGGGGGAAGCGGTGGAGGAGGGTCAGAGCCCAA |
| | | AAGCTCCGACAAGACTCACACATGCCCCCCTTGTCCAG |
| | | CGCCTGAAGCTGAGGGTGCGCCCTCTGTCTTCCTTTTC |
| | | CCCCCTAAGCCGAAAGATACCCTGATGATCTCCCGCAC |
| | | TCCCGAAGTCACATGTGTTGTTGTCGACGTATCTCATG |
| | | AAGATCCTGAGGTGAAATTCAACTGGTATGTAGACGG |
| | | GGTCGAAGTTCATAATGCTAAGACTAAGCCACGAGAA |
| | | GAGCAATACAACTCAACGTATCGGGTGGTGAGCGTTC |
| | | TGACGGTTCTGCACCAAGATTGGCTTAATGGAAAAGA |
| | | GTATAAGTGCAAGGTGTCCAACAAGGCTCTTCCGGCA |
| | | CCCATCGAAAAGACGATTTCCAAAGCGAAAGGCCAAC |
| | | CTAGGGAACCGCAAGTTTACACTTTGCCCCCGTCAAGA |
| | | GACGAACTTACCAAGAATCAAGTTTCCCTGACGTGCCT |
| | | TGTGAAGGGCTTCTACCCTAGCGATATAGCAGTTGAGT |
| | | GGGAATCTAACGGCCAGCCCGAAAATAATTATAAGAC |
| | | TACTCCGCCCGTGCTGGACAGTGATGGTTCATTTTTCC |
| | | TGTATTCAAAACTCACTGTGGACAAATCTAGATGGCA |
| | | GCAGGGTAATGTGTTCTCTTGTTCAGTTATGCACGAGG |
| | | CATTGCACAATCACTATACGCAAAAAAGTTTGTCTCTC |
| | | TCTCCGGGG |
| SEQ ID NO: 211 | TACI(528)-Fc_589 | AGTCTCTCTTGCAGGAAAGAGCAAGGCGAATATTACG |
| | TACI 26 GSG4S | ACCATCTTTTGCGAGACTGTATAAGCTGTGCGTCTATT |
| | | TGTGGACAACACCCTAAACAATGTGCTGATTTCTGTGA |
| | | AAATAAGCTTCGATCTGGATCCGGTGGAGGAGGGTCA |
| | | GAGCCCAAAAGCTCCGACAAGACTCACACATGCCCCC |
| | | CTTGTCCAGCGCCTGAAGCTGAGGGTGCGCCCTCTGTC |
| | | TTCCTTTTCCCCCCTAAGCCGAAAGATACCCTGATGAT |
| | | CTCCCGCACTCCCGAAGTCACATGTGTTGTTGTCGACG |
| | | TATCTCATGAAGATCCTGAGGTGAAATTCAACTGGTAT |
| | | GTAGACGGGGTCGAAGTTCATAATGCTAAGACTAAGC |
| | | CACGAGAAGAGCAATACAACTCAACGTATCGGGTGGT |
| | | GAGCGTTCTGACGGTTCTGCACCAAGATTGGCTTAATG |
| | | GAAAAGAGTATAAGTGCAAGGTGTCCAACAAGGCTCT |
| | | TCCGGCACCCATCGAAAAGACGATTTCCAAAGCGAAA |
| | | GGCCAACCTAGGGAACCGCAAGTTTACACTTTGCCCCC |
| | | GTCAAGAGACGAACTTACCAAGAATCAAGTTTCCCTG |
| | | ACGTGCCTTGTGAAGGGCTTCTACCCTAGCGATATAGC |
| | | AGTTGAGTGGGAATCTAACGGCCAGCCCGAAAATAAT |
| | | TATAAGACTACTCCGCCCGTGCTGGACAGTGATGGTTC |
| | | ATTTTTCCTGTATTCAAAACTCACTGTGGACAAATCTA |
| | | GATGGCAGCAGGGTAATGTGTTCTCTTGTTCAGTTATG |
| | | CACGAGGCATTGCACAATCACTATACGCAAAAAAGTT |
| | | TGTCTCTCTCTCCGGGG |
| SEQ ID NO: 212 | (74) Fc 73 (G4S)4 | AGTCTCTCTTGCAGGAAAGAGCAAGGCGAATATTACG |
| | (84) TACI 26 | ACCATCTTTTGCGAGACTGTATAAGCTGTGCGTCTATT |
| | | TGTGGACAACACCCTAAACAATGTGCTGATTTCTGTGA |
| | | AAATAAGCTTCGATCTGGATCCGGTGGAGGAGGGTCA |
| | | GAGCCCAAAAGCTCCGACAAGACTCACACATGCCCCC |
| | | CTTGTCCAGCGCCTGAAGCTGAGGGTGCGCCCTCTGTC |
| | | TTCCTTTTCCCCCCTAAGCCGAAAGATACCCTGATGAT |
| | | CTCCCGCACTCCCGAAGTCACATGTGTTGTTGTCGACG |
| | | TATCTCATGAAGATCCTGAGGTGAAATTCAACTGGTAT |
| | | GTAGACGGGGTCGAAGTTCATAATGCTAAGACTAAGC |
| | | CACGAGAAGAGCAATACAACTCAACGTATCGGGTGGT |
| | | GAGCGTTCTGACGGTTCTGCACCAAGATTGGCTTAATG |
| | | GAAAAGAGTATAAGTGCAAGGTGTCCAACAAGGCTCT |
| | | TCCGGCACCCATCGAAAAGACGATTTCCAAAGCGAAA |
| | | GGCCAACCTAGGGAACCGCAAGTTTACACTTTGCCCCC |
| | | GTCAAGAGACGAACTTACCAAGAATCAAGTTTCCCTG |
| | | ACGTGCCTTGTGAAGGGCTTCTACCCTAGCGATATAGC |
| | | AGTTGAGTGGGAATCTAACGGCCAGCCCGAAAATAAT |
| | | TATAAGACTACTCCGCCCGTGCTGGACAGTGATGGTTC |
| | | ATTTTTCCTGTATTCAAAACTCACTGTGGACAAATCTA |
| | | GATGGCAGCAGGGTAATGTGTTCTCTTGTTCAGTTATG |
| | | CACGAGGCATTGCACAATCACTATACGCAAAAAAGTT |
| | | TGTCTCTCTCTCCGGGGGGGGGTGGCGGATCAGGAGG |
| | | TGGCGGTtcaGGCGGAGGAGGttctGGGGGTGGGGGTTCT |
| | | TCATTGTCCTGCAGAAAGGAACAGGGGGAGTATTACG |
| | | ATCACTTGCTTAGAGATTGTATAAGCTGCGCGAGCATT |
| | | TGCGGGCAACACCCTAAACAGTGTGCGGATTTCTGCG |
| | | AGAATAAACTCCGGTCT |
| SEQ ID NO: 213 | TACI WT 13 | AGTCTCTCTTGCAGGAAAGAGCAAGGCAAATTTTACG |
| | GSG4S (74) Fc | ACCATCTTTTGCGAGACTGTATAAGCTGTGCGTCTATT |
| | 73(G4S)4 (84) | TGTGGACAACACCCTAAACAATGTGCTTATTTCTGTGA |
| | (TACI WT 13 | AAATAAGCTTCGATCTGGATCCGGTGGAGGAGGGTCA |
| | | GAGCCCAAAAGCTCCGACAAGACTCACACATGCCCCC |
| | | CTTGTCCAGCGCCTGAAGCTGAGGGTGCGCCCTCTGTC |
| | | TTCCTTTTCCCCCCTAAGCCGAAAGATACCCTGATGAT |
| | | CTCCCGCACTCCCGAAGTCACATGTGTTGTTGTCGACG |
| | | TATCTCATGAAGATCCTGAGGTGAAATTCAACTGGTAT |
| | | GTAGACGGGGTCGAAGTTCATAATGCTAAGACTAAGC |
| | | CACGAGAAGAGCAATACAACTCAACGTATCGGGTGGT |
| | | GAGCGTTCTGACGGTTCTGCACCAAGATTGGCTTAATG |
| | | GAAAAGAGTATAAGTGCAAGGTGTCCAACAAGGCTCT |
| | | TCCGGCACCCATCGAAAAGACGATTTCCAAAGCGAAA |
| | | GGCCAACCTAGGGAACCGCAAGTTTACACTTTGCCCCC |
| | | GTCAAGAGACGAACTTACCAAGAATCAAGTTTCCCTG |
| | | ACGTGCCTTGTGAAGGGCTTCTACCCTAGCGATATAGC |
| | | AGTTGAGTGGGAATCTAACGGCCAGCCCGAAAATAAT |
| | | TATAAGACTACTCCGCCCGTGCTGGACAGTGATGGTTC |
| | | ATTTTTCCTGTATTCAAAACTCACTGTGGACAAATCTA |
| | | GATGGCAGCAGGGTAATGTGTTCTCTTGTTCAGTTATG |
| | | CACGAGGCATTGCACAATCACTATACGCAAAAAAGTT |
| | | TGTCTCTCTCTCCGGGGGGGGGTGGCGGAtcaGGAGGTG |
| | | GCGGTtcaGGCGGAGGAGGttctGGGGGTGGGGGTTCTTC |
| | | ATTGTCCTGCAGAAAGGAACAGGGGAAATTTTACGAT |
| | | CACTTGCTTAGAGATTGTATAAGCTGCGCGAGCATTTG |
| | | CGGGCAACACCCTAAACAGTGTGCGTATTTCTGCGAG |
| | | AATAAACTCCGGTCT |
| SEQ ID NO: 214 | TACI WT 1 | GTGGCAATGCGCTCATGCCCAGAGGAACAATATTGGG |
| | GS(G4S)2 (194) | ATCCGCTTCTTGGGACGTGTATGAGCTGCAAGACCATC |
| | scFc 218 | TGTAATCATCAATCCCAAAGGACATGCGCAGCTTTCTG |
| | | CAGGAGTCTCTCTTGCAGGAAAGAGCAAGGCAAATTT |
| | | TACGACCATCTTTTGCGAGACTGTATAAGCTGTGCGTC |
| | | TATTTGTGGACAACACCCTAAACAATGTGCTTATTTCT |
| | | GTGAAAATAAGCTTCGATCTGGATCCGGTGGTGGCGG |
| | | GTCAGGGGGAGGTGGAAGCGAGCCCAAGTCCTCAGAT |
| | | AAGACTCATACTTGCCCCCCCTGTCCCGCCCCTGAGGC |
| | | CGAAGGCGCACCGTCAGTGTTTTTGTTCCCACCAAAGC |
| | | CTAAGGACACACTGATGATAAGCAGAACACCTGAGGT |
| | | AACCTGTGTTGTTGTGGATGTAAGCCACGAAGATCCG |
| | | GAGGTTAAGTTTAACTGGTACGTGGACGGTGTAGAAG |
| | | TCCATAATGCTAAGACGAAGCCGAGGGAGGAACAATA |
| | | CAACTCCACGTATAGAGTGGTCTCCGTCTTGACCGTAC |
| | | TCCATCAAGACTGGCTCAACGGGAAAGAGTACAAGTG |
| | | TAAAGTCTCTAACAAAGCTCTTCCTGCACCGATTGAGA |
| | | AAACCATATCTAAAGCCAAGGGACAACCGAGAGAACC |
| | | ACAGGTTTACACGCTCCCCCCCAGTAGAGACGAACTT |
| | | ACAAAAAACCAGGTCAGTCTTACCTGCCTCGTCAAAG |
| | | GCTTCTACCCCTCTGACATCGCTGTTGAGTGGGAATCT |
| | | AACGGCCAACCTGAGAACAATTACAAAACAACCCCGC |
| | | CTGTCCTTGACTCAGACGGTTCCTTTTTTCTTTACAGCA |
| | | AGCTGACCGTCGACAAATCACGGTGGCAACAAGGCAA |
| | | TGTGTTTTCCTGTTCCGTGATGCACGAGGCACTGCACA |
| | | ACCACTACACTCAAAAATCCCTTTCCCTTTCCCCAGGG |
| | | GGAGGTGGAGGGAGCGGTGGAGGTGGTAGCGGGGGT |
| | | GGAGGCTCAGGTGGTGGGGGTTCCGGCGGTGGCGGAA |
| | | GTGGAGGCGGTGGCTCTGGTGGTGGCGGATCTGGCGG |
| | | AGGAGGCAGCGGCGGAGGTGGGTCTGGGGGTGGAGG |
| | | CTCCGGAGGCGGGGGAAGCGGTGGAGGAGGGTCAGA |
| | | GCCCAAAAGCTCCGACAAGACTCACACATGCCCCCCT |
| | | TGTCCAGCGCCTGAAGCTGAGGGTGCGCCCTCTGTCTT |
| | | CCTTTTCCCCCCTAAGCCGAAAGATACCCTGATGATCT |
| | | CCCGCACTCCCGAAGTCACATGTGTTGTTGTCGACGTA |
| | | TCTCATGAAGATCCTGAGGTGAAATTCAACTGGTATGT |
| | | AGACGGGGTCGAAGTTCATAATGCTAAGACTAAGCCA |
| | | CGAGAAGAGCAATACAACTCAACGTATCGGGTGGTGA |
| | | GCGTTCTGACGGTTCTGCACCAAGATTGGCTTAATGGA |
| | | AAAGAGTATAAGTGCAAGGTGTCCAACAAGGCTCTTC |
| | | CGGCACCCATCGAAAAGACGATTTCCAAAGCGAAAGG |
| | | CCAACCTAGGGAACCGCAAGTTTACACTTTGCCCCCGT |
| | | CAAGAGACGAACTTACCAAGAATCAAGTTTCCCTGAC |
| | | GTGCCTTGTGAAGGGCTTCTACCCTAGCGATATAGCAG |
| | | TTGAGTGGGAATCTAACGGCCAGCCCGAAAATAATTA |
| | | TAAGACTACTCCGCCCGTGCTGGACAGTGATGGTTCAT |
| | | TTTTCCTGTATTCAAAACTCACTGTGGACAAATCTAGA |
| | | TGGCAGCAGGGTAATGTGTTCTCTTGTTCAGTTATGCA |
| | | CGAGGCATTGCACAATCACTATACGCAAAAAAGTTTG |
| | | TCTCTCTCTCCGGGG |
| SEQ ID NO: 215 | | GCAATGCGCTCATGCCCAGAGGAACAATATTGGGATC |
| | | CGCTTCTTGGGACGTGTATGAGCTGCAAGACCATCTGT |
| | | AATCATCAATCCCAAAGGACATGCGCAGCTTTCTGCA |
| | | GGAGTCTCTCTTGCAGGAAAGAGCAAGGCAAATTTTA |
| | | CGACCATCTTTTGCGAGACTGTATAAGCTGTGCGTCTA |
| | | TTTGTGGACAACACCCTAAACAATGTGCTTATTTCTGT |
| | | GAAAATAAGCTTCGATCTGAGCCCAAAAGCTCCGACA |
| | | AGACTCACACATGCCCCCCTTGTCCAGCGCCTGAAGCT |
| | | GAGGGTGCGCCCTCTGTCTTCCTTTTCCCCCCTAAGCC |
| | | GAAAGATACCCTGATGATCTCCCGCACTCCCGAAGTC |
| | | ACATGTGTTGTTGTCGACGTATCTCATGAAGATCCTGA |
| | | GGTGAAATTCAACTGGTATGTAGACGGGGTCGAAGTT |
| | | CATAATGCTAAGACTAAGCCACGAGAAGAGCAATACA |
| | | ACTCAACGTATCGGGTGGTGAGCGTTCTGACGGTTCTG |
| | | CACCAAGATTGGCTTAATGGAAAAGAGTATAAGTGCA |
| | | AGGTGTCCAACAAGGCTCTTCCGagctCCATCGAAAAGA |
| | | CGATTTCCAAAGCGAAAGGCCAACCTAGGGAACCGCA |
| | | AGTTTACACTTTGCCCCCGTCAAGAGACGAACTTACCA |
| | | AGAATCAAGTTTCCCTGACGTGCCTTGTGAAGGGCTTC |
| | | TACCCTAGCGATATAGCAGTTGAGTGGGAATCTAACG |
| | | GCCAGCCCGAAAATAATTATAAGACTACTCCGCCCGT |
| | | GCTGGACAGTGATGGTTCATTTTTCCTGTATTCAAAAC |
| | | TCACTGTGGACAAATCTAGATGGCAGCAGGGTAATGT |
| | | GTTCTCTTGTTCAGTTATGCACGAGGCATTGCACAATC |
| | | ACTATACGCAAAAAAGTTTGTCTCTCTCTCCGGGGaag |
| SEQ ID NO: 216 | TACI WT 131 | agccgtgtggaccaggaggagcgctttccacagggcctgtgg |
| | GS(G4S)2 (194) | acgggggtgGCAA |
| | scFc_218 | TGCGCTCATGCCCAGAGGAACAATATTGGGATCCGCTT |
| | | CTTGGGACGTGTATGAGCTGCAAGACCATCTGTAATCA |
| | | TCAATCCCAAAGGACATGCGCAGCTTTCTGCAGGAGT |
| | | CTCTCTTGCAGGAAAGAGCAAGGCAAATTTTACGACC |
| | | ATCTTTTGCGAGACTGTATAAGCTGTGCGTCTATTTGT |
| | | GGACAACACCCTAAACAATGTGCTTATTTCTGTGAAAA |
| | | TAAGCTTCGATCTcccgtgaacctgccacccgagctgGGATC |
| | | CGGTGGTGGCGGGTCAGGGGGAGGTGGAAGCGAGCCCAAG |
| | | TCCTCAGATAAGACTCATACTTGCCCCCCCTGTCCCGC |
| | | CCCTGAGGCCGAAGGCGCACCGTCAGTGTTTTTGTTCC |
| | | CACCAAAGCCTAAGGACACACTGATGATAAGCAGAAC |
| | | ACCTGAGGTAACCTGTGTTGTTGTGGATGTAAGCCACG |
| | | AAGATCCGGAGGTTAAGTTTAACTGGTACGTGGACGG |
| | | TGTAGAAGTCCATAATGCTAAGACGAAGCCGAGGGAG |
| | | GAACAATACAACTCCACGTATAGAGTGGTCTCCGTCTT |
| | | GACCGTACTCCATCAAGACTGGCTCAACGGGAAAGAG |
| | | TACAAGTGTAAAGTCTCTAACAAAGCTCTTCCTGCACC |
| | | GATTGAGAAAACCATATCTAAAGCCAAGGGACAACCG |
| | | AGAGAACCACAGGTTTACACGCTCCCCCCCAGTAGAG |
| | | ACGAACTTACAAAAAACCAGGTCAGTCTTACCTGCCTC |
| | | GTCAAAGGCTTCTACCCCTCTGACATCGCTGTTGAGTG |
| | | GGAATCTAACGGCCAACCTGAGAACAATTACAAAACA |
| | | ACCCCGCCTGTCCTTGACTCAGACGGTTCCTTTTTTCTT |
| | | TACAGCAAGCTGACCGTCGACAAATCACGGTGGCAAC |
| | | AAGGCAATGTGTTTTCCTGTTCCGTGATGCACGAGGCA |
| | | CTGCACAACCACTACACTCAAAAATCCCTTTCCCTTTC |
| | | CCCAGGGGGAGGTGGAGGGAGCGGTGGAGGTGGTAG |
| | | CGGGGGTGGAGGCTCAGGTGGTGGGGGTTCCGGCGGT |
| | | GGCGGAAGTGGAGGCGGTGGCTCTGGTGGTGGCGGAT |
| | | CTGGCGGAGGAGGCAGCGGCGGAGGTGGGTCTGGGG |
| | | GTGGAGGCTCCGGAGGCGGGGGAAGCGGTGGAGGAG |
| | | GGTCAGAGCCCAAAAGCTCCGACAAGACTCACACATG |
| | | CCCCCCTTGTCCAGCGCCTGAAGCTGAGGGTGCGCCCT |
| | | CTGTCTTCCTTTTCCCCCCTAAGCCGAAAGATACCCTG |
| | | ATGATCTCCCGCACTCCCGAAGTCACATGTGTTGTTGT |
| | | CGACGTATCTCATGAAGATCCTGAGGTGAAATTCAACT |
| | | GGTATGTAGACGGGGTCGAAGTTCATAATGCTAAGAC |
| | | TAAGCCACGAGAAGAGCAATACAACTCAACGTATCGG |
| | | GTGGTGAGCGTTCTGACGGTTCTGCACCAAGATTGGCT |
| | | TAATGGAAAAGAGTATAAGTGCAAGGTGTCCAACAAG |
| | | GCTCTTCCGGCACCCATCGAAAAGACGATTTCCAAAG |
| | | CGAAAGGCCAACCTAGGGAACCGCAAGTTTACACTTT |
| | | GCCCCCGTCAAGAGACGAACTTACCAAGAATCAAGTT |
| | | TCCCTGACGTGCCTTGTGAAGGGCTTCTACCCTAGCGA |
| | | TATAGCAGTTGAGTGGGAATCTAACGGCCAGCCCGAA |
| | | AATAATTATAAGACTACTCCGCCCGTGCTGGACAGTG |
| | | ATGGTTCATTTTTCCTGTATTCAAAACTCACTGTGGAC |
| | | AAATCTAGATGGCAGCAGGGTAATGTGTTCTCTTGTTC |
| | | AGTTATGCACGAGGCATTGCACAATCACTATACGCAA |
| | | AAAAGTTTGTCTCTCTCTCCGGGG |
| SEQ ID NO: 217 | | agccgtgtggaccaggaggagcgctttccacagggcctgtgg |
| | | acgggggtgGCAA |
| | | TGCGCTCATGCCCAGAGGAACAATATTGGGATCCGCTT |
| | | CTTGGGACGTGTATGAGCTGCAAGACCATCTGTAATCA |
| | | TCAATCCCAAAGGACATGCGCAGCTTTCTGCAGGAGT |
| | | CTCTCTTGCAGGAAAGAGCAAGGCAAATTTTACGACC |
| | | ATCTTTTGCGAGACTGTATAAGCTGTGCGTCTATTTGT |
| | | GGACAACACCCTAAACAATGTGCTTATTTCTGTGAAAA |
| | | TAAGCTTCGATCTcccgtgaacctgccacccgagctgGACAA |
| | | GACTCACACATGCCCCCCTTGTCCAGCGCCTGAAGCTGAGG |
| | | GTGCGCCCTCTGTCTTCCTTTTCCCCCCTAAGCCGAAA |
| | | GATACCCTGATGATCTCCCGCACTCCCGAAGTCACATG |
| | | TGTTGTTGTCGACGTATCTCATGAAGATCCTGAGGTGA |
| | | AATTCAACTGGTATGTAGACGGGGTCGAAGTTCATAA |
| | | TGCTAAGACTAAGCCACGAGAAGAGCAATACAACTCA |
| | | ACGTATCGGGTGGTGAGCGTTCTGACGGTTCTGCACCA |
| | | AGATTGGCTTAATGGAAAAGAGTATAAGTGCAAGGTG |
| | | TCCAACAAGGCTCTTCCGagctCCATCGAAAAGACGATT |
| | | TCCAAAGCGAAAGGCCAACCTAGGGAACCGCAAGTTT |
| | | ACACTTTGCCCCCGTCAAGAGACGAACTTACCAAGAA |
| | | TCAAGTTTCCCTGACGTGCCTTGTGAAGGGCTTCTACC |
| | | CTAGCGATATAGCAGTTGAGTGGGAATCTAACGGCCA |
| | | GCCCGAAAATAATTATAAGACTACTCCGCCCGTGCTG |
| | | GACAGTGATGGTTCATTTTTCCTGTATTCAAAACTCAC |
| | | TGTGGACAAATCTAGATGGCAGCAGGGTAATGTGTTC |
| | | TCTTGTTCAGTTATGCACGAGGCATTGCACAATCACTA |
| | | TACGCAAAAAAGTTTGTCTCTCTCTCCGGGGaag |
| SEQ ID NO: 218 | single chain Fc | EPKSSDKTHTCPPCPAPEAEGAPSVFLFPPKPKDTLMISRT |
| | | PEVTCVVVDVSHEDPEVKFNWYVDGVEVHNAKTKPREE |
| | | QYNSTYRVVSVLTVLHQDWLNGKEYKCKVSNKALPAPI |
| | | EKTISKAKGQPREPQVYTLPPSRDELTKNQVSLTCLVKGF |
| | | YPSDIAVEWESNGQPENNYKTTPPVLDSDGSFFLYSKLTV |
| | | DKSRWQQGNVFSCSVMHEALHNHYTQKSLSLSPGGGGG |
| | | SGGGGSGGGGSGGGGSGGGGSGGGGSGGGGSGGGGSG |
| | | GGGSGGGGSGGGGSGGGGSEPKSSDKTHTCPPCPAPEAE |
| | | GAPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPG |
| SEQ ID NO: 219 | IgG2 Fc | VECPPCPAPPVAGPSVFLFPPKPKDTLMISRTPEVTCVVV |
| | | DVSHEDPEVQFNWYVDGVEVHNAKTKPREEQFNSTFRV |
| | | VSVLTVVHQDWLNGKEYKCKVSNKGLPAPIEKTISKTKG |
| | | QPREPQVYTLPPSREEMTKNQVSLTCLVKGFYPSDISVE |
| | | WESNGQPENNYKTTPPMLDSDGSFFLYSKLTVDKSRWQ |
| | | QGNVFSCSVMHEALHNHYTQKSLSLSPG |
| SEQ ID NO: 220 | IgG4 Fc S228P | ESKYGPPCPPCPAPEFLGGPSVFLFPPKPKDTLMISRTPEV |
| | | TCVVVDVSQEDPEVQFNWYVDGVEVHNAKTKPREEQF |
| | | NSTYRVVSVLTVLHQDWLNGKEYKCKVSNKGLPSSIEKT |
| | | ISKAKGQPREPQVYTLPPSQEEMTKNQVSLTCLVKGFYPS |
| | | DIAVEWESNGQPENNYKTTPPVLDSDGSFFLYSRLTVDK |
| | | SRWQEGNVFSCSVMHEALHNHYTQKSLSLSLG |
| SEQ ID NO: 221 | IgG1 Fc effectorless | DKTHTCPPCPAPEAEGAPSVFLFPPKPKDTLMISRTPEVTC |
| | (L234A/L235E/G237A) | VVVDVSHEDPEVKFNWYVDGVEVHNAKTKPREEQYNS |
| | and K447del | TYRVVSVLTVLHQDWLNGKEYKCKVSNKALPAPIEKTIS |
| | | KAKGQPREPQVYTLPPSRDELTKNQVSLTCLVKGFYPSDI |
| | | AVEWESNGQPENNYKTTPPVLDSDGSFFLYSKLTVDKSR |
| | | WQQGNVFSCSVMHEALHNHYTQKSLSLSPG |
| SEQ ID NO: 222 | Q75E | SLSCRKEEGKFYDHLLRDCISCASICGQHPKQCAYFCENK |
| | | LRSGSGGGGSEPKSSDKTHTCPPCPAPEAEGAPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALPAPIEKTISKAKGQPREPQVYTLPPSRDELTK |
| | | NQVSLTCLVKGFYPSDIAVEWESNGQPENNYKTTPPVLD |
| | | SDGSFFLYSKLTVDKSRWQQGNVFSCSVMHEALHNHYT |
| | | QKSLSLSPG |
| SEQ ID NO: 223 | Q75E, K77E | SLSCRKEEGEFYDHLLRDCISCASICGQHPKQCAYFCENK |
| | | LRSGSGGGGSEPKSSDKTHTCPPCPAPEAEGAPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALPAPIEKTISKAKGQPREPQVYTLPPSRDELTK |
| | | NQVSLTCLVKGFYPSDIAVEWESNGQPENNYKTTPPVLD |
| | | SDGSFFLYSKLTVDKSRWQQGNVFSCSVMHEALHNHYT |
| | | QKSLSLSPG |
| SEQ ID NO: 224 | Q75E, F78Y | SLSCRKEEGKYYDHLLRDCISCASICGQHPKQCAYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 225 | Q75E, A101D | SLSCRKEEGKFYDHLLRDCISCASICGQHPKQCDYFCENK |
| | | LRSGSGGGGSEPKSSDKTHTCPPCPAPEAEGAPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALPAPIEKTISKAKGQPREPQVYTLPPSRDELTK |
| | | NQVSLTCLVKGFYPSDIAVEWESNGQPENNYKTTPPVLD |
| | | SDGSFFLYSKLTVDKSRWQQGNVFSCSVMHEALHNHYT |
| | | QKSLSLSPG |
| SEQ ID NO: 226 | Q75E, Y102D | SLSCRKEEGKFYDHLLRDCISCASICGQHPKQCADFCENK |
| | | LRSGSGGGGSEPKSSDKTHTCPPCPAPEAEGAPSVFLFPP |
| | | KPKDTLMISRTPEVTCVVVDVSHEDPEVKFNWYVDGVE |
| | | VHNAKTKPREEQYNSTYRVVSVLTVLHQDWLNGKEYK |
| | | CKVSNKALPAPIEKTISKAKGQPREPQVYTLPPSRDELTK |
| | | NQVSLTCLVKGFYPSDIAVEWESNGQPENNYKTTPPVLD |
| | | SDGSFFLYSKLTVDKSRWQQGNVFSCSVMHEALHNHYT |
| | | QKSLSLSPG |
| SEQ ID NO: 227 | K77E, F78Y, R84Q | SLSCRKEQGEYYDHLLQDCISCASICGQHPKQCAYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 228 | F78Y | SLSCRKEQGKYYDHLLRDCISCASICGQHPKQCAYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 229 | F78Y, R84Q | SLSCRKEQGKYYDHLLQDCISCASICGQHPKQCAYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 230 | F78Y, A101D | SLSCRKEQGKYYDHLLRDCISCASICGQHPKQCDYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 231 | F78Y, Y102D | SLSCRKEQGKYYDHLLRDCISCASICGQHPKQCADFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 232 | R84Q, A101D | SLSCRKEQGKFYDHLLQDCISCASICGQHPKQCDYFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 233 | R84Q, Y102D | SLSCRKEQGKFYDHLLQDCISCASICGQHPKQCADFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 234 | A101D, Y102D | SLSCRKEQGKFYDHLLRDCISCASICGQHPKQCDDFCEN |
| | | KLRSGSGGGGSEPKSSDKTHTCPPCPAPEAEGAPSVFLFP |
| | | PKPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDELT |
| | | KNQVSLTCLVKGFYPSDIAVEWESNGQPENNYKTTPPVL |
| | | DSDGSFFLYSKLTVDKSRWQQGNVFSCSVMHEALHNHY |
| | | TQKSLSLSPG |
| SEQ ID NO: 235 | vTACI (Y102D)-Fc | SLSCRKEQGKFYDHLLRDCISCASICGQHPKQCADFCEN |
| | | KLRS |
| | | GSGGGGS |
| | | EPKSSDKTHTCPPCP |
| | | APEAEGAPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAK |
| | | GQPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| SEQ ID NO: 236 | vTACI | SLSCRKEQGEYYDHLLRDCISCASICGQHPKQCAYFCEN |
| | (K77E,F87Y)-Fc | KLRS |
| | | GSGGGGS |
| | | EPKSSDKTHTCPPCP |
| | | APEAEGAPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAK |
| | | GQPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| SEQ ID NO: 237 | vTACI | SLSCRKEQGEFYDHLLRDCISCASICGQHPKQCADFCENK |
| | (K77E,Y102D)-Fc | LRS |
| | | GSGGGGS |
| | | EPKSSDKTHTCPPCP |
| | | APEAEGAPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAK |
| | | GQPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| SEQ ID NO: 238 | hinge | EPKSS |
| SEQ ID NO: 239 | hinge | EPKSC |
| SEQ ID NO: 240 | TACI 13-118-Fc- | SRVDQEERFPQGLWTGVAMRSCPEEQYWDPLLGTCMSC |
| | 81 | KTICNHQSQRTCAAFCRSLSCRKEQGKFYDHLLRDCISCA |
| | | SICGQHPKQCAYFCENKLRSPVNLPPELDKPHTCPLCPAP |
| | | ELLGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPAPIEKTISKAKGQPREPQV |
| | | YTLPPSRDELTKNQVSLTCLVKGFYPSDIAVEWESNGQPE |
| | | NNYKATPPVLDSDGSFFLYSKLTVDK |
| | | SRWQQGNVFSCSVMHEALHNHYTQKSLSLSPGK |
| SEQ ID NO: 241 | TACI 13-118-Fc | SRVDQEERFPQGLWTGVAMRSCPEEQYWDPLLGTCMSC |
| | | KTICNHQSQRTCAAFCRSLSCRKEQGKFYDHLLRDCISCA |
| | | SICGQHPKQCAYFCENKLRSPVNLPPELDKTHTCPPCPAP |
| | | EAEGAPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPSSIEKTISKAKGQPREPQV |
| | | YTLPPSRDELTKNQVSLTCLVKGFYPSDIAVEWESNGQPE |
| | | NNYKTTPPVLDSDGSFFLYSKLTVDK |
| | | SRWQQGNVFSCSVMHEALHNHYTQKSLSLSPGK |

1. A formulation comprising a TACI-Fc fusion protein, comprising:
a. 135 mM to 165 mM of a tonicifying agent, wherein the tonicifying agent comprises arginine;
b. an acetate buffer; and
c. 0.002% to 1.0% (w/v) of a non-ionic surfactant;
wherein the TACI-Fc fusion protein is at a concentration of 100 mg/mL to 200 mg/mL; and
the formulation has a pH of 4.6 to 4.8.

2. A formulation comprising a TACI-Fc fusion protein, comprising:
a. 1% to 4% (w/v) proline;
b. an acetate buffer; and
c. 0.002% to 1.0% (w/v) of a non-ionic surfactant;
wherein the TACI-Fc fusion protein is at a concentration of 100 mg/mL to 200 mg/mL; and
the formulation has a pH of 4.7 to 5.7.

3. (canceled)

4. A formulation comprising a TACI-Fc fusion protein, comprising:
a. water and a buffering agent that provides a formulation pH of 4.3 to 5.5;
b. a tonicifying agent having a positive charge at the formulation pH;
c. a non-ionic surfactant; and
d. a TACI-Fc fusion protein at a concentration of 100 mg/mL to 200 mg/mL.

5. The formulation of claim 1, wherein the tonicifying agent is present in the formulation at a concentration of 145 mM to 150 mM; 140 mM, 145 mM, 150 mM, or 155 mM.

6. The formulation of claim 1 comprising an isotonic tonicity of 250 to 350 mOsm/Kg, or of 250 mOsm/Kg to 600 mOsm/Kg.

7. (canceled)

8. The formulation of claim 1, wherein the formulation pH is 4.7.

9. The formulation of claim 1, wherein the TACI-Fc fusion protein is at a concentration of 150 mg/mL, 160 mg/mL, 170 mg/mL, 175 mg/mL, or 180 mg/mL.

10. The formulation of claim 1, comprising 3 mM to 15 mM, 4 mM to 20 mM, from 5 mM to 10 mM, 8 mM to 25 mM, 10 mM to 20 mM, or 7 mM to 25 mM acetate as a component of the acetate buffer.

11. (canceled)

12. (canceled)

13. The formulation of claim 1, comprising 0.1% (w/v) of the non-ionic surfactant, wherein the non-ionic surfactant is poloxamer 188.

14. (canceled)

15. A formulation comprising a TACI-Fc fusion protein, comprising:
a. 140 mM to 150 mM arginine;
b. an acetate buffer; and
c. 0.1% (w/v) poloxamer 188 (P188); wherein
the TACI-Fc fusion protein is at a concentration of 175 mg/mL; and
the formulation has a pH of 4.7.

16-19. (canceled)

20. The formulation of claim 1, wherein the TACI-Fc fusion protein comprises (i) a TACI polypeptide sequence comprising the amino acid sequence of SEQ ID NO: 26; and (ii) an immunoglobulin Fc region.

21. The formulation of claim 20, wherein the immunoglobulin Fe region comprises the amino acid sequence of any one of SEQ ID NOs: 73, 75, 83, 136, 175, 176, or 221.

22-25. (canceled)

26. The formulation of claim 1, wherein the TACI-Fc fusion protein is a homodimer comprising two identical copies of the TACI-Fc fusion protein set forth in SEQ ID NO: 167 linked by a covalent disulfide bond.

27. The formulation of claim 1, wherein the TACI-Fc fusion protein has an isoelectric point (pI) of 7.0 to 7.8, 7.0 to 7.7, 7.0 to 7.6, 7.0 to 7.5, 7.0 to 7.3, 7.1 to 7.5, 7.1 to 7.3, or of 7.2.

28. The formulation of claim 1, that is a liquid formulation that has not been lyophilized and is not lyophilized prior to administration to a subject.

29. The formulation of claim 1, wherein, after storage for 12 months in liquid form at a storage temperature of 2° C. to 8° C., optionally of 5° C., and as measured by size exclusion chromatography (SEC) and as expressed in terms of area % under a curve that includes the TACI-Fc fusion protein in a form of a homodimer comprising two identical copies of the TACI-Fc fusion protein linked by a covalent disulfide bond as a main species, wherein the formulation has the following properties:
the main species is at least 95%, at least 98%, or at least 99% of all observed species under the curve;
less than 5%, less than 2%, or less than 1% of species under the curve have a size greater than a size of the main species;
less than 5%, less than 2%, or less than 1% of species under the curve have a size less than a size of the main species;
the loss of the main species is less than 1%;
the increase in species under the curve having a size greater than a size of the main species is less than 1%; and/or
the increase in species under the curve having a size less than a size of the main species is less than 1%.

30. The formulation of claim 1, wherein the formulation is a liquid formulation having a viscosity of 3 cP to 15 cP at 25° C. as assessed using a rheometer or a cone and plate viscometer.

31. The formulation of claim 1, wherein the formulation is a liquid formulation comprising less than 6,000 particles/mL having a particle size diameter of ≥10 μm; and/or comprising less than-abet 600 particles/mL having a particle size diameter of ≥25 μm, after storage for 12 months in liquid form at a storage temperature of 2° C. to 8° C., optionally of 5° C.

32. An article of manufacture comprising the formulation of claim 1 in a container, optionally wherein the container is a vial or a syringe barrel.

33. A kit comprising the formulation of claim 1 and instructions for use.

34-50. (canceled)

51. The formulation of claim 1, wherein the formulation comprises a hydrochloride salt form of arginine.

52. The formulation of claim 51, wherein the formulation comprises arginine at a concentration of 2.7% to 3.5% (w/v).

53. The formulation of claim 1, wherein the TACI-Fc fusion protein is at a concentration of 50 mg/mL to 200 mg/ml, 150 mg/ml to 175 mg/ml, or 160 mg/ml to 175 mg/ml.
